(19) United States
(12) Patent Application Publication
(10) Pub. No.: US 2024/0360449 A1
(43) Pub. Date: Oct. 31, 2024

(54) COMPOUNDS AND METHODS FOR MODULATING SCN1A EXPRESSION

(71) Applicant: Ionis Pharmaceuticals, Inc., Carlsbad, CA (US)

(72) Inventors: Paymaan Jafar-nejad, Carlsbad, CA (US); Frank Rigo, Carlsbad, CA (US)

(73) Assignee: Ionis Pharmaceuticals, Inc., Carlsbad, CA (US)

(21) Appl. No.: 18/685,362

(22) PCT Filed: Aug. 26, 2022
(86) PCT No.: PCT/US2022/075493

§ 371 Date: Feb. 21, 2024

Related U.S. Application Data
(60) Provisional application No. 63/237,898, filed on Aug. 27, 2021.

(51) Int. Cl. C12N 15/113; A61K 31/7115; A61K 31/712; A61K 31/7125

(52) U.S. Cl. CPC C12N 15/113; A61K 31/7115; A61K 31/712; A61K 31/7125; C12N 2310/11; C12N 2310/315; C12N 2310/321; C12N 2310/3341; C12N 2320/33

(57) ABSTRACT

Provided are oligomeric compounds, methods, and pharmaceutical compositions for modulating expression of SCN1A RNA and/or protein in a cell or subject. Such compounds, methods, and pharmaceutical compositions are useful to ameliorate at least one symptom of a developmental or epileptic encephalopathic disease, such as, for example, Dravet Syndrome. Such symptoms include seizures, sudden unexpected death in epilepsy, status epilepticus, behavioral dysfunctions, movement and balance dysfunctions, orthopedic conditions, motor dysfunctions, cognitive impairment, delayed language and speech, visual motor integration dysfunctions, visual perception dysfunctions, executive dysfunctions, and dysautonomia.

### SEQUENCE LISTING

The present application is being filed along with a Sequence Listing in electronic format. The Sequence Listing is provided as a file entitled BIOL0440WOSEQ.xml, created on Aug. 22, 2022, which is 789 KB in size. The information in the electronic format of the sequence listing is incorporated herein by reference in its entirety.

### FIELD

Provided are oligomeric compounds, methods, and pharmaceutical compositions for modulating expression of SCN1A RNA and/or protein in a cell or subject. Such compounds, methods, and pharmaceutical compositions are useful to ameliorate at least one symptom of a developmental or epileptic encephalopathic disease, such as, for example, Dravet Syndrome. Such symptoms include seizures, sudden unexpected death in epilepsy, status epilepticus, behavioral dysfunctions, movement and balance dysfunctions, orthopedic conditions, motor dysfunctions, cognitive impairment, delayed language and speech, visual motor integration dysfunctions, visual perception dysfunctions, executive dysfunctions, and dysautonomia.

### BACKGROUND

The human gene SCN1A encodes human SCN1A protein, the alpha-1 subunit of the voltage-gated sodium channel NaV1.1. Mutations in SCN1A lead to developmental and epileptic encephalopathies (DEEs), including Dravet Syndrome (previously known as Severe Myoclonic Epilepsy of Infancy (SMEI)), one of the most severe childhood forms of epilepsy; other epileptic disorders, including, for example, Genetic Epilepsy with Febrile Seizures Plus (GEFS+) and other febrile seizures, Idiopathic/Generic Generalized Epilepsies (IGE/GGE), Temporal Lobe Epilepsy, Myoclonic Astatic Epilepsy (MAE), Lennox-Gastaut Syndrome, and Migrating Partial Epilepsy of Infancy (MMPSI); and familial hemiplegic migraines, with or without epilepsy (Harkin, L. A., et al., 2007, Brain 130, 843-852; Escayg, A., et al., 2010, Epilepsia 51, 1650-1658; Miller I. O, et al., 2007 Nov. 29 [Updated 2019 Apr. 18]. In: Adam M P, Ardinger H H, Pagon R A, et al., editors. GeneReviews® [Internet]. Seattle (WA): University of Washington, Seattle; 1993-2020. Available from: https://www.ncbi.nlm.nih.gov/books/NBK1318/).

DEEs are associated with SCN1A haploinsufficiency (Parihar, R., et al., 2013, J. Human Genetics, 58, 573-580). Symptoms associated with DEEs, including Dravet Syndrome, include seizures that are prolonged in duration (often lasting longer than 10 minutes), frequent seizures (for example, convulsive, myoclonic, absence, focal, obtundation status, and tonic seizures), sudden unexpected death in epilepsy, status epilepticus, behavioral dysfunctions (for example, aggressiveness, agitation, obsessiveness, preservation, hoarding behavior, or sleep disorders), movement and balance dysfunctions, orthopedic conditions, motor system dysfunctions (for example, ataxia, tremors, dysarthria, pyramidal, and extrapyramidal signs), cognitive impairment, delayed language and speech, visual motor integration dysfunctions, visual perception dysfunctions, executive dysfunctions, and dysautonomia. Dravet Syndrome patients experience additional neurodevelopmental delays, leading to severe neurological disability (Guzzetta, F., 2011, Epilepsia 52:S2, 35-38; Anwar et al., 2019, Cureus 11, e5006). Alternative splicing of SCN1A leads to multiple SCN1A transcript variants (Parihar, R., et al., 2013). Certain transcript variants include a nonsense-mediated decay-inducing exon (NIE) (Steward, C. A., et al., 2019, npj Genom. Med. 4, 31; Carvill et al., 2018, American J. Human Genetics, 103, 1022-1029). One such NIE (NIE-1), which is 64 nucleobases in length and located in SCN1A intron 20, causes degradation of the SCN1A transcript (Carvill et al., 2018).

Currently there remains a need for therapies to treat Dravet Syndrome, GEFS+, and other DEEs. It is therefore an object herein to provide oligomeric compounds, methods, and pharmaceutical compositions for the treatment of such diseases.

### SUMMARY

Provided herein are compounds, methods, and pharmaceutical compositions for modulating expression of SCN1A RNA and/or protein in a cell or a subject. In certain embodiments, the amount of SCN1A RNA and/or SCN1A protein is increased. In certain embodiments, the compounds, methods, or pharmaceutical compositions modulate splicing of SCN1A RNA. In certain embodiments, the amount of full-length SCN1A RNA and/or full-length SCN1A protein is increased. In certain embodiments, the amount of SCN1A RNA including an NIE is reduced. In certain embodiments, the amount of SCN1A RNA excluding an NIE is increased. In certain embodiments, the NIE is NIE-1. In certain embodiments, the compounds, methods, and pharmaceutical compositions are useful for treating a disease or disorder associated with SCN1A. In certain embodiments, the disease or disorder associated with SCN1A is an SCN1A haploinsufficiency. In certain embodiments, the disease or disorder associated with SCN1A is a developmental or epileptic encephalopathic disease (DEE). In certain embodiments, the developmental or epileptic encephalopathic disease is any of Genetic Epilepsy with Febrile Seizures Plus (GEFS+), febrile seizures, Idiopathic/Generic Generalized Epilepsies (IGE/GGE), Temporal Lobe Epilepsy, Myoclonic Astatic Epilepsy (MAE), Lennox-Gastaut Syndrome, or Migrating Partial Epilepsy of Infancy (MMPSI). In certain embodiments, the developmental or epileptic encephalopathic disease is Dravet Syndrome. In certain embodiments, the DEE is treated by increasing the amount of full-length SCN1A RNA and/or full-length SCN1A protein in a subject, or cell thereof, with compounds capable of excluding an NIE from an SCN1A RNA. In certain embodiments, exclusion of an NIE from an SCN1A RNA reduces or prevents degradation of the SCN1A transcript via the NMD pathway. In certain embodiments, exclusion of an NIE from an SCN1A RNA increases full-length SCN1A RNA and/or full-length SCN1A protein wherein removal of the NIE prevents degradation of the SCN1A transcript via the NMD pathway. In certain embodiments, compounds useful for modulating splicing of SCN1A RNA are oligomeric compounds. In certain embodiments, the oligomeric compound comprises or consists of a modified oligonucleotide.

Also provided are methods useful for ameliorating at least one symptom or hallmark of a disease or disorder associated with SCN1A. In some embodiments, the disease or disorder associated with SCN1A is a DEE. In certain embodiments, the DEE is Dravet Syndrome. In certain embodiments, symptoms or hallmarks of the DEE include seizures that are prolonged in duration (often lasting longer than 10 minutes), frequent seizures (for example, convulsive, myoclonic, absence, focal, obtundation status, and tonic seizures), sudden unexpected death in epilepsy, status epilepticus, behavioral dysfunctions (for example, aggressiveness, agitation, obsessiveness, preservation, hoarding behavior, or sleep disorders), movement and balance dysfunctions, orthopedic conditions, motor system dysfunctions (for example, ataxia, tremors, dysarthria, pyramidal, and extrapyramidal signs), cognitive impairment, delayed language and speech, visual motor integration dysfunctions, visual perception dysfunctions, executive dysfunctions, and dysautonomia

### DETAILED DESCRIPTION

It is to be understood that both the foregoing general description and the following detailed description are exemplary and explanatory only and are not restrictive. Herein, the use of the singular includes the plural unless specifically stated otherwise. As used herein, the use of “or” means “and/or” unless stated otherwise. Furthermore, the use of the term “including” as well as other forms, such as “includes” and “included”, is not limiting. Also, terms such as “element” or “component” encompass both elements and components comprising one unit and elements and components that comprise more than one subunit, unless specifically stated otherwise.

The section headings used herein are for organizational purposes only and are not to be construed as limiting the subject matter described. All documents, or portions of documents, cited in this application, including, but not limited to, patents, patent applications, articles, books, and treatises, and GenBank and NCBI reference sequence records are hereby expressly incorporated-by-reference for the portions of the document discussed herein, as well as in their entirety.

### Definitions

Unless specific definitions are provided, the nomenclature used in connection with, and the procedures and techniques of, analytical chemistry, synthetic organic chemistry, and medicinal and pharmaceutical chemistry described herein are those well-known and commonly used in the art. Where permitted, all patents, applications, published applications and other publications and other data referred to throughout in the disclosure are incorporated by reference herein in their entirety.

Unless otherwise indicated, the following terms have the following meanings:

As used herein, “2′-deoxynucleoside” means a nucleoside comprising a 2′-H(H) deoxyfuranosyl sugar moiety. In certain embodiments, a 2′-deoxynucleoside is a 2′-β-D-deoxynucleoside and comprises a 2′-β-D-deoxyribosyl sugar moiety, which has the β-D ribosyl configuration as found in naturally occurring deoxyribonucleic acids (DNA). In certain embodiments, a 2′-deoxynucleoside may comprise a modified nucleobase or may comprise an RNA nucleobase (uracil).

As used herein, “2′-MOE” means a 2′-O(CH₂)₂OCH₃ group in place of the 2′-OH group of a furanosyl sugar moiety. A “2′-MOE sugar moiety” or a “2′-O-methoxyethyl sugar moiety” means a sugar moiety with a 2′-OCH₂CH₂OCH₃ group in place of the 2′-OH group of a furanosyl sugar moiety. Unless otherwise indicated, a 2′-MOE sugar moiety is in the β-D-ribosyl configuration. “MOE” means O-methoxyethyl.

As used herein, “2′-MOE nucleoside” means a nucleoside comprising a 2′-MOE sugar moiety.

As used herein, “2′-NMA” means a —O—CH₂—C(═O)—NH—CH₃ group in place of the 2′-OH group of a furanosyl sugar moiety. A “2′-NMA sugar moiety” means a sugar moiety with a 2′-O—CH₂—C(═O)—NH—CH₃ group in place of the 2′-OH group of a furanosyl sugar moiety. Unless otherwise indicated, a 2′-NMA sugar moiety is in the β-D configuration. “NMA” means O—N-methyl acetamide.

As used herein, “2′-NMA nucleoside” means a nucleoside comprising a 2′-NMA sugar moiety.

As used herein, “2′-substituted nucleoside” means a nucleoside comprising a 2′-substituted furanosyl sugar moiety. As used herein, “2′-substituted” in reference to a sugar moiety means a sugar moiety comprising at least one 2′-substituent group other than H or OH.

As used herein, “5-methylcytosine” means a cytosine modified with a methyl group attached to the 5 position.

A 5-methylcytosine is a modified nucleobase.

As used herein, “administering” means providing a pharmaceutical agent to a subject.

As used herein, “ameliorate” in reference to a treatment means improvement in at least one symptom or hallmark relative to the same symptom or hallmark in the absence of the treatment. In certain embodiments, amelioration is the reduction in the severity or frequency of a symptom or hallmark or the delayed onset or slowing of progression in the severity or frequency of a symptom or hallmark. In certain embodiments, the symptom or hallmark is seizures that are prolonged in duration (often lasting longer than 10 minutes), frequent seizures (for example, convulsive, myoclonic, absence, focal, obtundation status, and tonic seizures), sudden unexpected death in epilepsy, status epilepticus, behavioral dysfunctions (for example, aggressiveness, agitation, obsessiveness, preservation, hoarding behavior, or sleep disorders), and developmental delays, movement and balance dysfunctions, orthopedic conditions, motor system and cognitive dysfunctions (for example, ataxia, tremors, dysarthria, pyramidal, and extrapyramidal signs), cognitive impairment, delayed language and speech issues, visual motor integration dysfunctions, visual perception dysfunctions, executive dysfunctions, growth and nutrition issues, sleeping difficulties, chronic infections, sensory integration disorders, or dysautonomia.

As used herein, “cerebrospinal fluid” or “CSF” means the fluid filling the space around the brain and spinal cord. “Artificial cerebrospinal fluid” or “aCSF” means a prepared or manufactured fluid that has certain properties (e.g., osmolarity, pH, and/or electrolytes) of cerebrospinal fluid and is biocompatible with CSF.

As used herein, “conjugate group” means a group of atoms that is directly attached to an oligonucleotide. Conjugate groups include a conjugate moiety and a conjugate linker that attaches the conjugate moiety to the oligonucleotide.

As used herein, “conjugate linker” means a single bond or a group of atoms comprising at least one bond that connects a conjugate moiety to an oligonucleotide.

As used herein, “conjugate moiety” means a group of atoms that modifies one or more properties of a molecule compared to the identical molecule lacking the conjugate moiety, including but not limited to pharmacodynamics, pharmacokinetics, stability, binding, absorption, tissue distribution, cellular distribution, cellular uptake, charge and clearance.

As used herein, “internucleoside linkage” is the covalent linkage between adjacent nucleosides in an oligonucleotide. As used herein “modified internucleoside linkage” means any internucleoside linkage other than a phosphodiester internucleoside linkage.

As used herein, “linked nucleosides” are nucleosides that are connected in a contiguous sequence (i.e., no additional nucleosides are presented between those that are linked).

As used herein, “linker-nucleoside” means a nucleoside that links, either directly or indirectly, an oligonucleotide to a conjugate moiety. Linker-nucleosides are located within the conjugate linker of an oligomeric compound. Linker-nucleosides are not considered part of the oligonucleotide portion of an oligomeric compound even if they are contiguous with the oligonucleotide.

As used herein, “motif” means the pattern of unmodified and/or modified sugar moieties, nucleobases, and/or internucleoside linkages, in an oligonucleotide.

As used herein, “nonsense-mediated decay-inducing exon (NIE)” is an exon, or a pseudo-exon, that, when included in an mRNA transcript can activate the nonsense-mediated decay (NMD) pathway. “NIE-1” is a 64 nucleobase in length NIE located in intron 20 of the SCN1A gene (chr2:166863579-166864271, hg19; Carvill et al., 2018), which, when present in the transcript, causes degradation of the SCN1A transcript. In certain embodiments, human NIE-1 has the nucleobase sequence of SEQ ID NO: 16. In certain embodiments, mouse NIE-1 has the nucleobase sequence of SEQ ID NO: 17.

As used herein, “modified nucleoside” means a nucleoside comprising a modified nucleobase and/or a modified sugar moiety.

As used herein, “nucleobase” means an unmodified nucleobase or a modified nucleobase. A nucleobase is a heterocyclic moiety. As used herein an “unmodified nucleobase” is adenine (A), thymine (T), cytosine (C), uracil (U), or guanine (G). As used herein, a “modified nucleobase” is a group of atoms other than unmodified A, T, C, U, or G capable of pairing with at least one other nucleobase. A “5-methylcytosine” is a modified nucleobase. A universal base is a modified nucleobase that can pair with any one of the five unmodified nucleobases.

As used herein, “nucleobase sequence” means the order of contiguous nucleobases in a nucleic acid or oligonucleotide independent of any sugar or internucleoside linkage modification.

As used herein, “nucleoside” means a compound or fragment of a compound comprising a nucleobase and a sugar moiety. The nucleobase and sugar moiety are each, independently, unmodified or modified.

As used herein, “oligomeric compound” means an oligonucleotide and optionally one or more additional features, such as a conjugate group or terminal group. An oligomeric compound may be paired with a second oligomeric compound that is complementary to the first oligomeric compound or may be unpaired. A “singled-stranded oligomeric compound” is an unpaired oligomeric compound.

The term “oligomeric duplex” means a duplex formed by two oligomeric compounds having complementary nucleobase sequences.

As used herein, “oligonucleotide” means a strand of linked nucleosides connected via internucleoside linkages, wherein each nucleoside and internucleoside linkage may be modified or unmodified. Unless otherwise indicated, oligonucleotides consist of 8-50 linked nucleosides. As used herein, “modified oligonucleotide” means an oligonucleotide, wherein at least one nucleoside or internucleoside linkage is modified. As used herein, “unmodified oligonucleotide” means an oligonucleotide that does not comprise any nucleoside modifications or internucleoside modifications.

As used herein, “pharmaceutically acceptable carrier or diluent” means any substance suitable for use in administering to an animal. Certain such carriers enable pharmaceutical compositions to be formulated as, for example, tablets, pills, dragees, capsules, liquids, gels, syrups, slurries, suspension, and lozenges for the oral ingestion by a subject. In certain embodiments, a pharmaceutically acceptable diluent is sterile water, sterile saline, sterile buffer solution or sterile artificial cerebrospinal fluid.

As used herein “pharmaceutically acceptable salts” means physiologically and pharmaceutically acceptable salts of compounds. Pharmaceutically acceptable salts retain the desired biological activity of the parent compound and do not impart undesired toxicological effects thereto.

As used herein “pharmaceutical composition” means a mixture of substances suitable for administering to a subject. For example, a pharmaceutical composition may comprise an oligomeric compound and a sterile aqueous solution. In certain embodiments, a pharmaceutical composition shows activity in free uptake assay in certain cell lines.

As used herein, “stereorandom” or “stereorandom chiral center” in the context of a population of molecules of identical molecular formula means a chiral center that is not controlled during synthesis, or enriched following synthesis, for a particular absolute stereochemical configuration. The stereochemical configuration of a chiral center is random when it is the result of a synthetic method that is not designed to control the stereochemical configuration. For example, in a population of molecules comprising a stereorandom chiral center, the number of molecules having the (S) configuration of the stereorandom chiral center may be but is not necessarily the same as the number of molecules having the (R) configuration of the stereorandom chiral center (“racemic”). In certain embodiments, the stereorandom chiral center is not racemic because one absolute configuration predominates following synthesis, e.g., due to the action of non-chiral reagents near the enriched stereochemistry of an adjacent sugar moiety. In certain embodiments, a stereorandom chiral center is a stereorandom phosphorothioate internucleoside linkage.

As used herein, “subject” means a human or non-human animal.

As used herein, “sugar moiety” means an unmodified sugar moiety or a modified sugar moiety. As used herein, “unmodified sugar moiety” means a 2′-OH(H) ribosyl moiety, as found in RNA (an “unmodified RNA sugar moiety”), or a 2′-H(H) deoxyribosyl sugar moiety, as found in DNA (an “unmodified DNA sugar moiety”). Unmodified sugar moieties have one hydrogen at each of the 1′, 3′, and 4′ positions, an oxygen at the 3′ position, and two hydrogens at the 5′ position. As used herein, “modified sugar moiety” or “modified sugar” means a modified furanosyl sugar moiety or a sugar surrogate.

As used herein, “sugar surrogate” means a modified sugar moiety that can link a nucleobase to another group, such as an internucleoside linkage, conjugate group, or terminal group in an oligonucleotide, but which is not a furanosyl sugar moiety or a bicyclic sugar moiety. Modified nucleosides comprising sugar surrogates can be incorporated into one or more positions within an oligonucleotide and such oligonucleotides are capable of hybridizing to complementary oligomeric compounds or target nucleic acids. Examples of sugar surrogates include GNA (glycol nucleic acid), FHNA (fluoro hexitol nucleic acid), morpholino, and other structures described herein and known in the art.

As used herein, “symptom or hallmark” means any physical feature or test result that indicates the existence or extent of a disease or disorder. In certain embodiments, a symptom is apparent to a subject or to a medical professional examining or testing said subject. In certain embodiments, a hallmark is apparent upon invasive diagnostic testing, including, but not limited to, post-mortem tests. In certain embodiments, a hallmark is apparent on a brain MRI scan.

As used herein, “target nucleic acid” and “target RNA” mean a nucleic acid that an oligomeric compound is designed to affect. Target RNA means an RNA transcript and includes pre-mRNA and mRNA unless otherwise specified.

As used herein, “target region” means a portion of a target nucleic acid to which an oligomeric compound is designed to hybridize.

As used herein, “terminal group” means a chemical group or group of atoms that is covalently linked to a terminus of an oligonucleotide.

As used herein, “antisense activity” means any detectable and/or measurable change attributable to the hybridization of an antisense compound to its target nucleic acid. In certain embodiments, antisense activity is a decrease in the amount or expression of a target nucleic acid or protein encoded by such target nucleic acid compared to target nucleic acid levels or target protein levels in the absence of the antisense compound. In certain embodiments, antisense activity is the modulation of splicing of a target pre-mRNA.

As used herein, “antisense agent” means an antisense compound and optionally one or more additional features, such as a sense compound.

As used herein, “antisense compound” means an antisense oligonucleotide and optionally one or more additional features, such as a conjugate group.

As used herein, “sense compound” means a sense oligonucleotide and optionally one or more additional features, such as a conjugate group.

As used herein, “antisense oligonucleotide” means an oligonucleotide, including the oligonucleotide portion of an antisense compound, that is capable of hybridizing to a target nucleic acid and is capable of at least one antisense activity. Antisense oligonucleotides include but are not limited to antisense RNAi oligonucleotides and antisense RNase H oligonucleotides.

As used herein, “sense oligonucleotide” means an oligonucleotide, including the oligonucleotide portion of a sense compound, that is capable of hybridizing to an antisense oligonucleotide.

As used herein, “hybridization” means the annealing of oligonucleotides and/or nucleic acids. While not limited to a particular mechanism, the most common mechanism of hybridization involves hydrogen bonding, which may be Watson-Crick, Hoogsteen or reversed Hoogsteen hydrogen bonding, between complementary nucleobases. In certain embodiments, complementary nucleic acid molecules include, but are not limited to, an antisense compound and a nucleic acid target. In certain embodiments, complementary nucleic acid molecules include, but are not limited to, an oligonucleotide and a nucleic acid target.

As used herein, “treating” means improving a subject's disease or condition by administering an oligomeric compound described herein. In certain embodiments, treating a subject improves a symptom relative to the same symptom in the absence of the treatment. In certain embodiments, treatment reduces in the severity or frequency of a symptom, or delays the onset of a symptom, slows the progression of a symptom, or slows the severity or frequency of a symptom.

As used herein, “therapeutically effective amount” means an amount of a pharmaceutical agent or composition that provides a therapeutic benefit to a subject. For example, a therapeutically effective amount improves a symptom of a disease.

### CERTAIN EMBODIMENTS

Embodiment 1. An oligomeric compound comprising a modified oligonucleotide according to the following chemical notation:

(SEQ ID NO: 23)
AₙₛGₙₒTₙₒTₙₛGₙₛGₙₛAₙₛGₙₛ^{m}CₙₛAₙₛAₙₛGₙₛAₙₛTₙₛTₙₛAₙₛTₙₛ^{m}Cₙ;
(SEQ ID NO: 24)
AₙₛGₙₒTₙₛTₙₒGₙₛGₙₛAₙₛGₙₛ^{m}CₙₛAₙₛAₙₛGₙₛAₙₛTₙₛTₙₛAₙₛTₙₛ^{m}Cₙ;
(SEQ ID NO: 25)
AₙₛGₙₒTₙₛTₙₛGₙₒGₙₛAₙₛGₙₛ^{m}CₙₛAₙₛAₙₛGₙₛAₙₛTₙₛTₙₛAₙₛTₙₛ^{m}Cₙ;
(SEQ ID NO: 26)
AₙₛGₙₒTₙₛTₙₛGₙₛGₙₒAₙₛGₙₛ^{m}CₙₛAₙₛAₙₛGₙₛAₙₛTₙₛTₙₛAₙₛTₙₛ^{m}Cₙ;
(SEQ ID NO: 27)
AₙₛGₙₒTₙₛTₙₛGₙₛGₙₛAₙₛGₙₒ^{m}CₙₛAₙₛAₙₛGₙₛAₙₛTₙₛTₙₛAₙₛTₙₛ^{m}Cₙ;
(SEQ ID NO: 28)
AₙₛGₙₛTₙₛTₙₒGₙₒGₙₛAₙₛGₙₛ^{m}CₙₛAₙₛAₙₛGₙₛAₙₛTₙₛTₙₛAₙₛTₙₛ^{m}Cₙ;
(SEQ ID NO: 29)
AₙₛGₙₛTₙₛTₙₛGₙₛGₙₛAₙₛGₙₒ^{m}CₙₒAₙₛAₙₛGₙₛAₙₛTₙₛTₙₛAₙₛTₙₛ^{m}Cₙ;
(SEQ ID NO: 30)
AₙₛGₙₛTₙₛTₙₛGₙₛGₙₛAₙₛGₙₛ^{m}CₙₛAₙₒAₙₒGₙₛAₙₛTₙₛTₙₛAₙₛTₙₛ^{m}Cₙ;
(SEQ ID NO: 31)
AₙₛGₙₛTₙₛTₙₛGₙₛGₙₛAₙₛGₙₛ^{m}CₙₛAₙₛAₙₛGₙₒAₙₒTₙₛTₙₛAₙₛTₙₛ^{m}Cₙ;
(SEQ ID NO: 32)
AₙₛGₙₒTₙₛTₙₛGₙₛGₙₛAₙₛGₙₛ^{m}CₙₛAₙₒAₙₛGₙₛAₙₛTₙₛTₙₛAₙₛTₙₛ^{m}Cₙ;
(SEQ ID NO: 33)
AₙₛGₙₒTₙₛTₙₛGₙₛGₙₛAₙₛGₙₛ^{m}CₙₛAₙₛAₙₛGₙₒAₙₛTₙₛTₙₛAₙₛTₙₛ^{m}Cₙ;
(SEQ ID NO: 34)
AₙₛGₙₒTₙₛTₙₛGₙₛGₙₛAₙₛGₙₛ^{m}CₙₛAₙₛAₙₛGₙₛAₙₛTₙₛTₙₒAₙₛTₙₛ^{m}Cₙ;
(SEQ ID NO: 35)
AₙₛGₙₛTₙₛTₙₛGₙₛGₙₒAₙₒGₙₛ^{m}CₙₛAₙₛAₙₛGₙₛAₙₛTₙₛTₙₛAₙₛTₙₛ^{m}Cₙ;
(SEQ ID NO: 36)
AₙₛGₙₛTₙₛTₙₛGₙₛGₙₛAₙₛGₙₛ^{m}CₙₛAₙₛAₙₛGₙₛAₙₛTₙₒTₙₒAₙₛTₙₛ^{m}Cₙ;
(SEQ ID NO: 37)
AₙₛGₙₛTₙₛTₙₛGₙₛGₙₛAₙₒGₙₛ^{m}CₙₛAₙₛAₙₛGₙₛAₙₛTₙₛTₙₒAₙₛTₙₛ^{m}Cₙ;
(SEQ ID NO: 38)
AₙₛGₙₛTₙₛTₙₛGₙₛGₙₛAₙₛGₙₛ^{m}CₙₒAₙₛAₙₛGₙₛAₙₛTₙₛTₙₒAₙₛTₙₛ^{m}Cₙ;
(SEQ ID NO: 39)
AₙₛGₙₛTₙₛTₙₛGₙₛGₙₛAₙₛGₙₛ^{m}CₙₛAₙₛAₙₒGₙₛAₙₛTₙₛTₙₒAₙₛTₙₛ^{m}Cₙ;
(SEQ ID NO: 40)
AₙₛGₙₛTₙₛTₙₛGₙₛGₙₛAₙₛGₙₛ^{m}CₙₛAₙₛAₙₛGₙₛAₙₒTₙₛTₙₒAₙₛTₙₛ^{m}Cₙ;
(SEQ ID NO: 41)
AₙₛGₙₛTₙₛTₙₛGₙₛGₙₛAₙₛGₙₛ^{m}CₙₒAₙₛAₙₛGₙₛAₙₛTₙₒTₙₛAₙₛTₙₛ^{m}Cₙ;
(SEQ ID NO: 42)
AₙₛGₙₛTₙₛTₙₛGₙₛGₙₛAₙₛGₙₛ^{m}CₙₛAₙₛAₙₛGₙₒAₙₛTₙₒTₙₛAₙₛTₙₛ^{m}Cₙ;
(SEQ ID NO: 43)
AₙₒAₙₛGₙₛTₙₛTₙₛGₙₛGₙₛAₙₛGₙₛ^{m}CₙₛAₙₛAₙₛGₙₛAₙₛTₙₛTₙₛAₙₛTₙₛ^{m}Cₙ;
or
(SEQ ID NO: 44)
AₙₒAₙₛGₙₛTₙₛTₙₛGₙₛGₙₛAₙₛGₙₛ^{m}CₙₛAₙₛAₙₛGₙₛAₙₛTₙₛTₙₛAₙₛTₙₛ^{m}Cₙₒ^{m}Cₙ;

wherein:
   A=an adenine nucleobase,
   ^{m}C=a 5-methylcytosine nucleobase,
   G=a guanine nucleobase,
   T=a thymine nucleobase,
   n=a 2′-NMA sugar moiety,
   s=a phosphorothioate internucleoside linkage, and
   o=a phosphodiester internucleoside linkage.

Embodiment 2. The oligomeric compound of embodiment 1, consisting of the modified oligonucleotide.

Embodiment 3. The oligomeric compound of embodiment 1 or embodiment 2, wherein the modified oligonucleotide is a free acid.

Embodiment 4. The oligomeric compound of embodiment 1 or embodiment 2, wherein the modified oligonucleotide is a salt.

Embodiment 5. The oligomeric compound of embodiment 4, wherein the modified oligonucleotide is a sodium salt or a potassium salt.

Embodiment 6. An oligomeric compound comprising a modified oligonucleotide consisting of 17 to 30 linked nucleosides and having a nucleobase sequence comprising at least 12, at least 13, at least 14, at least 15, at least 16, at least 17, at least 18, at least 19, at least 20, at least 21, at least 22, at least 23, at least 24, or at least 25 consecutive nucleobases of any of the nucleobase sequences of SEQ ID NOs:19-22 or 63-86, wherein the modified oligonucleotide comprises at least one modification selected from a modified sugar moiety and a modified internucleoside linkage.

Embodiment 7. The oligomeric compound of embodiment 6, wherein the modified oligonucleotide consists of 18-25 linked nucleosides.

Embodiment 8. The oligomeric compound of any embodiments 6-7, wherein the modified oligonucleotide consists of 18, 23 or 25 linked nucleosides.

Embodiment 9. The oligomeric compound according to embodiment 6, wherein the nucleobase sequence of the modified oligonucleotide comprises the nucleobase sequence of any of SEQ ID Nos: 19-22 or 63-86.

Embodiment 10. The oligomeric compound according to embodiment 6, wherein the nucleobase sequence of the modified oligonucleotide consists of the nucleobase sequence of any of SEQ ID Nos:19-22 or 63-86.

Embodiment 11. The oligomeric compound according to any of embodiments 6-10, wherein the modified oligonucleotide comprises at least one modified sugar moiety.

Embodiment 12. The oligomeric compound of any of embodiment 11, wherein the modified oligonucleotide comprises at least one non-bicyclic modified sugar moiety.

Embodiment 13. The oligomeric compound of embodiment 12, wherein the non-bicyclic modified sugar moiety is a 2′-MOE sugar moiety or a 2′-NMA sugar moiety.

Embodiment 14. The oligomeric compound of any of embodiments 11-13, wherein each nucleoside of the modified oligonucleotide comprises a modified sugar moiety.

Embodiment 15. The oligomeric compound of any of embodiments 11-14, wherein each modified sugar moiety is a 2′-NMA sugar moiety.

Embodiment 16. The oligomeric compound of any of embodiments 6-15, wherein the modified oligonucleotide comprises at least one modified internucleoside linkage.

Embodiment 17. The oligomeric compound of embodiment 16, wherein the at least one modified internucleoside linkage is a phosphorothioate internucleoside linkage.

Embodiment 18. The oligomeric compound of embodiment 16 or embodiment 17, wherein the modified oligonucleotide comprises at least one phosphodiester internucleoside linkage.

Embodiment 19. The oligomeric compound of any of embodiments 16-18, wherein each internucleoside linkage is independently selected from a phosphodiester internucleoside linkage and a phosphorothioate internucleoside linkage.

Embodiment 20. The oligomeric compound of any of embodiments 16, 17, or 19, wherein each internucleoside linkage is a phosphorothioate internucleoside linkage.

Embodiment 21. The oligomeric compound of any of embodiments 6-20, wherein the modified oligonucleotide comprises at least one modified nucleobase.

Embodiment 22. The oligomeric compound of embodiment 21, wherein the modified nucleobase is a 5-methyl cytosine Embodiment 23. An oligomeric compound comprising a modified oligonucleotide according to the following chemical notation:

(SEQ ID NO: 45)
GₙₛGₙₛTₙₒAₙₒGₙₛ^{m}CₙₛAₙₛAₙₛAₙₛAₙₛGₙₛGₙₛGₙₛGₙₛTₙₛAₙₛAₙₛTₙₛAₙₛ^{m}CₙₛAₙₛGₙₛTₙ;
(SEQ ID NO: 46)
GₙₛGₙₛTₙₛAₙₛGₙₛ^{m}CₙₛAₙₛAₙₛAₙₛAₙₛGₙₛGₙₛGₙₛGₙₛTₙₛAₙₛAₙₛTₙₛAₙₛ^{m}CₙₛAₙₛGₙₛTₙ;
(SEQ ID NO: 47)
AₙₛTₙₛ^{m}Cₙₒ^{m}CₙₒAₙₛAₙₒGₙₒTₙₛTₙₒGₙₒGₙₛAₙₛGₙₛ^{m}CₙₛAₙₛAₙₛGₙₛAₙₛTₙₛTₙₛAₙₛTₙₛ^{m}Cₙₛ^{m}CₙₛTₙ;
(SEQ ID NO: 48)
Tₙₛ^{m}Cₙₛ^{m}CₙₒAₙₒAₙₛGₙₒTₙₒTₙₛGₙₒGₙₒAₙₛGₙₛ^{m}CₙₛAₙₛAₙₛGₙₛAₙₛTₙₛTₙₛAₙₛTₙₛ^{m}Cₙₛ^{m}CₙₛTₙₛAₙ;
(SEQ ID NO: 49)
^{m}Cₙₛ^{m}CₙₛAₙₒAₙₒGₙₛTₙₒTₙₒGₙₛGₙₒAₙₒGₙₛ^{m}CₙₛAₙₛAₙₛGₙₛAₙₛTₙₛTₙₛAₙₛTₙₛ^{m}Cₙₛ^{m}CₙₛTₙₛAₙₛTₙ;
(SEQ ID NO: 50)
^{m}CₙₛAₙₛAₙₒGₙₒTₙₛTₙₒGₙₒGₙₛAₙₒGₙₒ^{m}CₙₛAₙₛAₙₛGₙₛAₙₛTₙₛTₙₛAₙₛTₙₛ^{m}Cₙₛ^{m}CₙₛTₙₛAₙₛTₙₛAₙ;
(SEQ ID NO: 51)
AₙₛTₙₛ^{m}Cₙₒ^{m}CₙₒAₙₛAₙₒGₙₒTₙₛTₙₛGₙₛGₙₛAₙₛGₙₛ^{m}CₙₛAₙₛAₙₛGₙₛAₙₛTₙₛTₙₛAₙₛTₙₛ^{m}Cₙₛ^{m}CₙₛTₙ;
(SEQ ID NO: 52)
Tₙₛ^{m}Cₙₛ^{m}CₙₒAₙₒAₙₛGₙₒTₙₒTₙₛGₙₛGₙₛAₙₛGₙₛ^{m}CₙₛAₙₛAₙₛGₙₛAₙₛTₙₛTₙₛAₙₛTₙₛ^{m}Cₙₛ^{m}CₙₛTₙₛAₙ;
(SEQ ID NO: 53)
^{m}Cₙₛ^{m}CₙₛAₙₒAₙₒGₙₛTₙₒTₙₒGₙₛGₙₛAₙₛGₙₛ^{m}CₙₛAₙₛAₙₛGₙₛAₙₛTₙₛTₙₛAₙₛTₙₛ^{m}Cₙₛ^{m}CₙₛTₙₛAₙₛTₙ;
(SEQ ID NO: 54)
^{m}CₙₛAₙₛAₙₒGₙₒTₙₛTₙₒGₙₒGₙₛAₙₛGₙₛ^{m}CₙₛAₙₛAₙₛGₙₛAₙₛTₙₛTₙₛAₙₛTₙₛ^{m}Cₙₛ^{m}CₙₛTₙₛAₙₛTₙₛAₙ;
(SEQ ID NO: 55)
AₙₛTₙₛ^{m}Cₙₒ^{m}CₙₒAₙₛAₙₛGₙₛTₙₛTₙₛGₙₛGₙₛAₙₛGₙₛ^{m}CₙₛAₙₛAₙₛGₙₛAₙₛTₙₛTₙₛAₙₛTₙₛ^{m}Cₙₛ^{m}CₙₛTₙ;
(SEQ ID NO: 56)
Tₙₛ^{m}Cₙₛ^{m}CₙₒAₙₒAₙₛGₙₛTₙₛTₙₛGₙₛGₙₛAₙₛGₙₛ^{m}CₙₛAₙₛAₙₛGₙₛAₙₛTₙₛTₙₛAₙₛTₙₛ^{m}Cₙₛ^{m}CₙₛTₙₛAₙ;
(SEQ ID NO: 57)
^{m}Cₙₛ^{m}CₙₛAₙₒAₙₒGₙₛTₙₛTₙₛGₙₛGₙₛAₙₛGₙₛ^{m}CₙₛAₙₛAₙₛGₙₛAₙₛTₙₛTₙₛAₙₛTₙₛ^{m}Cₙₛ^{m}CₙₛTₙₛAₙₛTₙ;
(SEQ ID NO: 58)
^{m}CₙₛAₙₛAₙₒGₙₒTₙₛTₙₛGₙₛGₙₛAₙₛGₙₛ^{m}CₙₛAₙₛAₙₛGₙₛAₙₛTₙₛTₙₛAₙₛTₙₛ^{m}Cₙₛ^{m}CₙₛTₙₛAₙₛTₙₛAₙ;
(SEQ ID NO: 59)
AₙₛTₙₛ^{m}Cₙₛ^{m}CₙₛAₙₛAₙₛGₙₛTₙₛTₙₛGₙₛGₙₛAₙₛGₙₛ^{m}CₙₛAₙₛAₙₛGₙₛAₙₛTₙₛTₙₛAₙₛTₙₛ^{m}Cₙₛ^{m}CₙₛTₙ;
(SEQ ID NO: 60)
Tₙₛ^{m}Cₙₛ^{m}CₙₛAₙₛAₙₛGₙₛTₙₛTₙₛGₙₛGₙₛAₙₛGₙₛ^{m}CₙₛAₙₛAₙₛGₙₛAₙₛTₙₛTₙₛAₙₛTₙₛ^{m}Cₙₛ^{m}CₙₛTₙₛAₙ;
(SEQ ID NO: 61)
^{m}Cₙₛ^{m}CₙₛAₙₛAₙₛGₙₛTₙₛTₙₛGₙₛGₙₛAₙₛGₙₛ^{m}CₙₛAₙₛAₙₛGₙₛAₙₛTₙₛTₙₛAₙₛTₙₛ^{m}Cₙₛ^{m}CₙₛTₙₛAₙₛTₙ;
or
(SEQ ID NO: 62)
^{m}CₙₛAₙₛAₙₛGₙₛTₙₛTₙₛGₙₛGₙₛAₙₛGₙₛ^{m}CₙₛAₙₛAₙₛGₙₛAₙₛTₙₛTₙₛAₙₛTₙₛ^{m}Cₙₛ^{m}CₙₛTₙₛAₙₛTₙₛAₙ,

wherein:
   A=an adenine nucleobase,
   ^{m}C=a 5-methylcytosine nucleobase,
   G=a guanine nucleobase,
   T=a thymine nucleobase,
   n=a 2′-NMA sugar moiety,
   s=a phosphorothioate internucleoside linkage, and
   o=a phosphodiester linkage.

Embodiment 24. The oligomeric compound of embodiment 23, consisting of the modified oligonucleotide.

Embodiment 25. The oligomeric compound of embodiment 23 or embodiment 24, wherein the modified oligonucleotide is a free acid.

Embodiment 26. The oligomeric compound of embodiment 23 or embodiment 24, wherein the modified oligonucleotide is a salt.

Embodiment 27. The oligomeric compound of embodiment 26, wherein the modified oligonucleotide is a sodium salt or a potassium salt.

Embodiment 28. A population of oligomeric compounds of any of embodiments 1-27, wherein all of the phosphorothioate internucleoside linkages of the modified oligonucleotide are stereorandom.

Embodiment 29. A pharmaceutical composition comprising an oligomeric compound of any of embodiments 1-27 or a population of oligomeric compounds of embodiment 28, and a pharmaceutically acceptable diluent.

Embodiment 30. The pharmaceutical composition of embodiment 29, wherein the pharmaceutically acceptable diluent is artificial cerebrospinal fluid (aCSF) or PBS.

Embodiment 31. The pharmaceutical composition of embodiment 29 or embodiment 30, wherein the pharmaceutical composition consists essentially of the oligomeric compound and aCSF or PBS.

Embodiment 32. The pharmaceutical composition of any of embodiments 29-31, wherein the pharmaceutical composition consists essentially of the population of modified oligonucleotides or the population of oligomeric compounds and aCSF or PBS.

Embodiment 33. A method comprising administering to a subject an oligomeric compound of any of embodiments 1-27, a population of oligomeric compounds of embodiment 28, or a pharmaceutical composition of any of embodiments 29-32.

Embodiment 34. A method of treating a disease associated with SCN1A comprising administering to a subject having a disease associated with SCN1A a therapeutically effective amount of an oligomeric compound of any of embodiments 1-27, a population of oligomeric compounds of embodiment 28, or a pharmaceutical composition of any of embodiments 29-32, thereby treating the disease associated with SCN1A.

Embodiment 35. The method of embodiment 34, wherein the disease associated with SCN1A is a developmental or epileptic encephalopathic disease.

Embodiment 36. The method of embodiment 35, wherein the developmental or epileptic encephalopathic disease is Dravet Syndrome.

Embodiment 37. The method of embodiment 35 or embodiment 36, wherein the developmental or epileptic encephalopathic disease is any of Genetic Epilepsy with Febrile Seizures Plus (GEFS+), febrile seizures, Idiopathic/Generic Generalized Epilepsies (IGE/GGE), Temporal Lobe Epilepsy, Myoclonic Astatic Epilepsy (MAE), Lennox-Gastaut Syndrome, or Migrating Partial Epilepsy of Infancy (MMPSI).

Embodiment 38. The method of any of embodiments 33-37, wherein administering the oligomeric compound, the population of oligomeric compounds, or the pharmaceutical composition reduces the frequency of seizures, reduces the duration of seizures, reduces status epilepticus, improves behavioral functions, improves movement and balance, improves orthopedic conditions, improves motor functions, reduces cognitive impairment, improves language and speech, improves visual motor integration functions, improvise visual perception functions, improves executive functions, or reduces dysautonomia.

Embodiment 39. The method of embodiment 38, wherein the seizures are frequent or prolonged in duration.

Embodiment 40. The method of embodiment 38 or embodiment 39, wherein the seizure is any of convulsive, myoclonic, absence, focal, obtundation status, or tonic.

Embodiment 41. The method of any of embodiments 33-40, wherein the frequency of seizures is reduced.

Embodiment 42. The method of any of embodiments 33-41, wherein the duration of seizures is reduced.

Embodiment 43. The method of any of embodiments 33-42, wherein the subject is human.

Embodiment 44. A method of increasing expression of SCN1A in a cell comprising contacting the cell with an oligomeric compound of any of embodiments 1-27, a population of oligomeric compounds of embodiment 28, or a pharmaceutical composition of any of embodiments 29-32.

Embodiment 45. A method of modulating splicing of an SCN1A RNA in a cell comprising contacting the cell with an oligomeric compound of any of embodiments 1-27.

Embodiment 46. The method of embodiment 45, wherein the amount of SCN1A RNA that includes an NIE is reduced.

Embodiment 47. The method of embodiment 45 or embodiment 46, wherein the amount of SCN1A RNA that includes NIE-1 is reduced.

Embodiment 48. The method of any of embodiments 45-47, wherein the amount of SCN1A RNA that excludes an NIE is increased.

Embodiment 49. The method of any of embodiments 45-48, wherein the amount of SCN1A RNA that excludes NIE-1 is increased.

Embodiment 50. The method of any of embodiments 45-49, wherein the cell is a cerebral cortex, hippocampus, brainstem, or thalamus cell.

Embodiment 51. The method of any of embodiments 45-50, wherein the cell is a human cell.

Embodiment 52. Use of an oligomeric compound of any of embodiments 1-27, a population of oligomeric compounds of embodiment 28, or a pharmaceutical composition of any of embodiments 29-32 for treating a disease associated with SCN1A.

Embodiment 53. Use of an oligomeric compound of any of embodiments 1-27 a population of oligomeric compounds of embodiment 28, or a pharmaceutical composition of any of embodiments 29-32 in the manufacture of a medicament for treating a disease associated with SCN1A.

Embodiment 54. The use of embodiment 52 or embodiment 53, wherein the disease associated with SCN1A is a developmental or epileptic encephalopathic disease.

Embodiment 55. The use of embodiment 54, wherein the developmental or epileptic encephalopathic disease is Dravet Syndrome.

Embodiment 56. The use of embodiment 54 or embodiment 55, wherein the developmental or epileptic encephalopathic disease is any of Genetic Epilepsy with Febrile Seizures Plus (GEFS+), febrile seizures, Idiopathic/Generic Generalized Epilepsies (IGE/GGE), Temporal Lobe Epilepsy, Myoclonic Astatic Epilepsy (MAE), Lennox-Gastaut Syndrome, or Migrating Partial Epilepsy of Infancy (MMPSI).

### Certain Compositions

In certain embodiments, compounds are represented by the chemical notations in the following table.

TABLE 1
Certain compositions
In certain embodiments, compounds are represented by the following chemical notations (5′ to 3′):

| Compound | | SEQ ID |
|---|---|---|
| Number | Chemical Notation (5′ to 3′) | NO |
| 1464713 | AₙₛGₙₒTₙₒTₙₛGₙₛGₙₛAₙₛGₙₛ^{m}CₙₛAₙₛAₙₛGₙₛAₙₛTₙₛTₙₛAₙₛTₙₛ^{m}Cₙ | 23 |
| 1464714 | AₙₛGₙₒTₙₛTₙₒGₙₛGₙₛAₙₛGₙₛ^{m}CₙₛAₙₛAₙₛGₙₛAₙₛTₙₛTₙₛAₙₛTₙₛ^{m}Cₙ | 24 |
| 1464717 | AₙₛGₙₒTₙₛTₙₛGₙₒGₙₛAₙₛGₙₛ^{m}CₙₛAₙₛAₙₛGₙₛAₙₛTₙₛTₙₛAₙₛTₙₛ^{m}Cₙ | 25 |
| 1464718 | AₙₛGₙₒTₙₛTₙₛGₙₛGₙₒAₙₛGₙₛ^{m}CₙₛAₙₛAₙₛGₙₛAₙₛTₙₛTₙₛAₙₛTₙₛ^{m}Cₙ | 26 |
| 1464719 | AₙₛGₙₒTₙₛTₙₛGₙₛGₙₛAₙₛGₙₒ^{m}CₙₛAₙₛAₙₛGₙₛAₙₛTₙₛTₙₛAₙₛTₙₛ^{m}Cₙ | 27 |
| 1464720 | AₙₛGₙₛTₙₛTₙₒGₙₒGₙₛAₙₛGₙₛ^{m}CₙₛAₙₛAₙₛGₙₛAₙₛTₙₛTₙₛAₙₛTₙₛ^{m}Cₙ | 28 |
| 1464721 | AₙₛGₙₛTₙₛTₙₛGₙₛGₙₛAₙₛGₙₒ^{m}CₙₒAₙₛAₙₛGₙₛAₙₛTₙₛTₙₛAₙₛTₙₛ^{m}Cₙ | 29 |
| 1464722 | AₙₛGₙₛTₙₛTₙₛGₙₛGₙₛAₙₛGₙₛ^{m}CₙₛAₙₒAₙₒGₙₛAₙₛTₙₛTₙₛAₙₛTₙₛ^{m}Cₙ | 30 |
| 1464723 | AₙₛGₙₛTₙₛTₙₛGₙₛGₙₛAₙₛGₙₛ^{m}CₙₛAₙₛAₙₛGₙₒAₙₒTₙₛTₙₛAₙₛTₙₛ^{m}Cₙ | 31 |
| 1594953 | AₙₛGₙₒTₙₛTₙₛGₙₛGₙₛAₙₛGₙₛ^{m}CₙₛAₙₒAₙₛGₙₛAₙₛTₙₛTₙₛAₙₛTₙₛ^{m}Cₙ | 32 |
| 1594954 | AₙₛGₙₒTₙₛTₙₛGₙₛGₙₛAₙₛGₙₛ^{m}CₙₛAₙₛAₙₛGₙₒAₙₛTₙₛTₙₛAₙₛTₙₛ^{m}Cₙ | 33 |
| 1594955 | AₙₛGₙₒTₙₛTₙₛGₙₛGₙₛAₙₛGₙₛ^{m}CₙₛAₙₛAₙₛGₙₛAₙₛTₙₛTₙₒAₙₛTₙₛ^{m}Cₙ | 34 |
| 1594956 | AₙₛGₙₛTₙₛTₙₛGₙₛGₙₒAₙₒGₙₛ^{m}CₙₛAₙₛAₙₛGₙₛAₙₛTₙₛTₙₛAₙₛTₙₛ^{m}Cₙ | 35 |
| 1594960 | AₙₛGₙₛTₙₛTₙₛGₙₛGₙₛAₙₛGₙₛ^{m}CₙₛAₙₛAₙₛGₙₛAₙₛTₙₒTₙₒAₙₛTₙₛ^{m}Cₙ | 36 |
| 1594962 | AₙₛGₙₛTₙₛTₙₛGₙₛGₙₛAₙₒGₙₛ^{m}CₙₛAₙₛAₙₛGₙₛAₙₛTₙₛTₙₒAₙₛTₙₛ^{m}Cₙ | 37 |
| 1594963 | AₙₛGₙₛTₙₛTₙₛGₙₛGₙₛAₙₛGₙₛ^{m}CₙₒAₙₛAₙₛGₙₛAₙₛTₙₛTₙₒAₙₛTₙₛ^{m}Cₙ | 38 |
| 1594964 | AₙₛGₙₛTₙₛTₙₛGₙₛGₙₛAₙₛGₙₛ^{m}CₙₛAₙₛAₙₒGₙₛAₙₛTₙₛTₙₒAₙₛTₙₛ^{m}Cₙ | 39 |
| 1594965 | AₙₛGₙₛTₙₛTₙₛGₙₛGₙₛAₙₛGₙₛ^{m}CₙₛAₙₛAₙₛGₙₛAₙₒTₙₛTₙₒAₙₛTₙₛ^{m}Cₙ | 40 |
| 1594966 | AₙₛGₙₛTₙₛTₙₛGₙₛGₙₛAₙₛGₙₛ^{m}CₙₒAₙₛAₙₛGₙₛAₙₛTₙₒTₙₛAₙₛTₙₛ^{m}Cₙ | 41 |
| 1594967 | AₙₛGₙₛTₙₛTₙₛGₙₛGₙₛAₙₛGₙₛ^{m}CₙₛAₙₛAₙₛGₙₒAₙₛTₙₒTₙₛAₙₛTₙₛ^{m}Cₙ | 42 |
| 1594968 | AₙₒAₙₛGₙₛTₙₛTₙₛGₙₛGₙₛAₙₛGₙₛ^{m}CₙₛAₙₛAₙₛGₙₛAₙₛTₙₛTₙₛAₙₛTₙₛ^{m}Cₙ | 43 |
| 1594969 | AₙₒAₙₛGₙₛTₙₛTₙₛGₙₛGₙₛAₙₛGₙₛ^{m}CₙₛAₙₛAₙₛGₙₛAₙₛTₙₛTₙₛAₙₛTₙₛ^{m}Cₙₒ^{m}Cₙ | 44 |
| 1669097 | GₙₛGₙₛTₙₒAₙₒGₙₛ^{m}CₙₛAₙₛAₙₛAₙₛAₙₛGₙₛGₙₛGₙₛGₙₛTₙₛAₙₛAₙₛTₙₛAₙₛ^{m}CₙₛAₙₛGₙₛTₙ | 45 |
| 1669102 | GₙₛGₙₛTₙₛAₙₛGₙₛ^{m}CₙₛAₙₛAₙₛAₙₛAₙₛGₙₛGₙₛGₙₛGₙₛTₙₛAₙₛAₙₛTₙₛAₙₛ^{m}CₙₛAₙₛGₙₛTₙ | 46 |
| 1669084 | AₙₛTₙₛ^{m}Cₙₒ^{m}CₙₒAₙₛAₙₒGₙₒTₙₛTₙₒGₙₒGₙₛAₙₛGₙₛ^{m}CₙₛAₙₛAₙₛGₙₛAₙₛTₙₛTₙₛAₙₛTₙₛ^{m}Cₙₛ^{m}CₙₛTₙ | 47 |
| 1669085 | Tₙₛ^{m}Cₙₛ^{m}CₙₒAₙₒAₙₛGₙₒTₙₒTₙₛGₙₒGₙₒAₙₛGₙₛ^{m}CₙₛAₙₛAₙₛGₙₛAₙₛTₙₛTₙₛAₙₛTₙₛ^{m}Cₙₛ^{m}CₙₛTₙₛAₙ | 48 |
| 1669086 | ^{m}Cₙₛ^{m}CₙₛAₙₒAₙₒGₙₛTₙₒTₙₒGₙₛGₙₒAₙₒGₙₛ^{m}CₙₛAₙₛAₙₛGₙₛAₙₛTₙₛTₙₛAₙₛTₙₛ^{m}Cₙₛ^{m}CₙₛTₙₛAₙₛTₙ | 49 |
| 1669087 | ^{m}CₙₛAₙₛAₙₒGₙₒTₙₛTₙₒGₙₒGₙₛAₙₒGₙₒ^{m}CₙₛAₙₛAₙₛGₙₛAₙₛTₙₛTₙₛAₙₛTₙₛ^{m}Cₙₛ^{m}CₙₛTₙₛAₙₛTₙₛAₙ | 50 |
| 1669088 | AₙₛTₙₛ^{m}Cₙₒ^{m}CₙₒAₙₛAₙₒGₙₒTₙₛTₙₛGₙₛGₙₛAₙₛGₙₛ^{m}CₙₛAₙₛAₙₛGₙₛAₙₛTₙₛTₙₛAₙₛTₙₛ^{m}Cₙₛ^{m}CₙₛTₙ | 51 |
| 1669089 | Tₙₛ^{m}Cₙₛ^{m}CₙₒAₙₒAₙₛGₙₒTₙₒTₙₛGₙₛGₙₛAₙₛGₙₛ^{m}CₙₛAₙₛAₙₛGₙₛAₙₛTₙₛTₙₛAₙₛTₙₛ^{m}Cₙₛ^{m}CₙₛTₙₛAₙ | 52 |
| 1669090 | ^{m}Cₙₛ^{m}CₙₛAₙₒAₙₒGₙₛTₙₒTₙₒGₙₛGₙₛAₙₛGₙₛ^{m}CₙₛAₙₛAₙₛGₙₛAₙₛTₙₛTₙₛAₙₛTₙₛ^{m}Cₙₛ^{m}CₙₛTₙₛAₙₛTₙ | 53 |
| 1669091 | ^{m}CₙₛAₙₛAₙₒGₙₒTₙₛTₙₒGₙₒGₙₛAₙₛGₙₛ^{m}CₙₛAₙₛAₙₛGₙₛAₙₛTₙₛTₙₛAₙₛTₙₛ^{m}Cₙₛ^{m}CₙₛTₙₛAₙₛTₙₛAₙ | 54 |
| 1669093 | AₙₛTₙₛ^{m}Cₙₒ^{m}CₙₒAₙₛAₙₛGₙₛTₙₛTₙₛGₙₛGₙₛAₙₛGₙₛ^{m}CₙₛAₙₛAₙₛGₙₛAₙₛTₙₛTₙₛAₙₛTₙₛ^{m}Cₙₛ^{m}CₙₛTₙ | 55 |
| 1669094 | Tₙₛ^{m}Cₙₛ^{m}CₙₒAₙₒAₙₛGₙₛTₙₛTₙₛGₙₛGₙₛAₙₛGₙₛ^{m}CₙₛAₙₛAₙₛGₙₛAₙₛTₙₛTₙₛAₙₛTₙₛ^{m}Cₙₛ^{m}CₙₛTₙₛAₙ | 56 |
| 1669095 | ^{m}Cₙₛ^{m}CₙₛAₙₒAₙₒGₙₛTₙₛTₙₛGₙₛGₙₛAₙₛGₙₛ^{m}CₙₛAₙₛAₙₛGₙₛAₙₛTₙₛTₙₛAₙₛTₙₛ^{m}Cₙₛ^{m}CₙₛTₙₛAₙₛTₙ | 57 |
| 1669096 | ^{m}CₙₛAₙₛAₙₒGₙₒTₙₛTₙₛGₙₛGₙₛAₙₛGₙₛ^{m}CₙₛAₙₛAₙₛGₙₛAₙₛTₙₛTₙₛAₙₛTₙₛ^{m}Cₙₛ^{m}CₙₛTₙₛAₙₛTₙₛAₙ | 58 |
| 1669098 | AₙₛTₙₛ^{m}Cₙₛ^{m}CₙₛAₙₛAₙₛGₙₛTₙₛTₙₛGₙₛGₙₛAₙₛGₙₛ^{m}CₙₛAₙₛAₙₛGₙₛAₙₛTₙₛTₙₛAₙₛTₙₛ^{m}Cₙₛ^{m}CₙₛTₙ | 59 |
| 1669099 | Tₙₛ^{m}Cₙₛ^{m}CₙₛAₙₛAₙₛGₙₛTₙₛTₙₛGₙₛGₙₛAₙₛGₙₛ^{m}CₙₛAₙₛAₙₛGₙₛAₙₛTₙₛTₙₛAₙₛTₙₛ^{m}Cₙₛ^{m}CₙₛTₙₛAₙ | 60 |
| 1669100 | ^{m}Cₙₛ^{m}CₙₛAₙₛAₙₛGₙₛTₙₛTₙₛGₙₛGₙₛAₙₛGₙₛ^{m}CₙₛAₙₛAₙₛGₙₛAₙₛTₙₛTₙₛAₙₛTₙₛ^{m}Cₙₛ^{m}CₙₛTₙₛAₙₛTₙ | 61 |
| 1669101 | ^{m}CₙₛAₙₛAₙₛGₙₛTₙₛTₙₛGₙₛGₙₛAₙₛGₙₛ^{m}CₙₛAₙₛAₙₛGₙₛAₙₛTₙₛTₙₛAₙₛTₙₛ^{m}Cₙₛ^{m}CₙₛTₙₛAₙₛTₙₛAₙ | 62 |

wherein,
A=an adenine nucleobase,
^{m}C=a 5-methylcytosine nucleobase,
G=a guanine nucleobase,
T=a thymine nucleobase,
n=a 2′-NMA sugar moiety,
s=a phosphorothioate internucleoside linkage, and
o=a phosphodiester internucleoside linkage.

### I. Certain Oligonucleotides

In certain embodiments, provided herein are oligomeric compounds comprising oligonucleotides, which consist of linked nucleosides. Oligonucleotides may be unmodified oligonucleotides (RNA or DNA) or may be modified oligonucleotides. Modified oligonucleotides comprise at least one modification relative to unmodified RNA or DNA. That is, modified oligonucleotides comprise at least one modified nucleoside (comprising a modified sugar moiety and/or a modified nucleobase) and/or at least one modified internucleoside linkage.

Certain embodiments provide an oligomeric compound comprising a modified oligonucleotide consisting of 17 to 30 linked nucleosides and having a nucleobase sequence comprising at least 12, at least 13, at least 14, at least 15, at least 16, at least 17, at least 18, at least 19, at least 20, at least 21, at least 22, at least 23, at least 24, or at least 25 consecutive nucleobases of any of the nucleobase sequences of SEQ ID NOs:19-22 or 63-86, wherein the modified oligonucleotide comprises at least one modification selected from a modified sugar moiety and a modified internucleoside linkage. In certain embodiments, the modified oligonucleotide consists of 18-25 linked nucleosides. In certain embodiments, the modified oligonucleotide consists of 18, 23 or 25 linked nucleosides.

Certain embodiments provide an oligomeric compound comprising a modified oligonucleotide comprising the nucleobase sequence of any of SEQ ID Nos: 19-22 or 63-86.

Certain embodiments provide an oligomeric compound comprising a modified oligonucleotide consisting of the nucleobase sequence of any of SEQ ID Nos: 19-22 or 63-86.

Certain embodiments provide an oligomeric compound comprising a modified oligonucleotide according to the following chemical notation:

(SEQ ID NO: 23)
AₙₛGₙₒTₙₒTₙₛGₙₛGₙₛAₙₛGₙₛ^{m}CₙₛAₙₛAₙₛGₙₛAₙₛTₙₛTₙₛAₙₛTₙₛ^{m}Cₙ;
(SEQ ID NO: 24)
AₙₛGₙₒTₙₛTₙₒGₙₛGₙₛAₙₛGₙₛ^{m}CₙₛAₙₛAₙₛGₙₛAₙₛTₙₛTₙₛAₙₛTₙₛ^{m}Cₙ;
(SEQ ID NO: 25)
AₙₛGₙₒTₙₛTₙₛGₙₒGₙₛAₙₛGₙₛ^{m}CₙₛAₙₛAₙₛGₙₛAₙₛTₙₛTₙₛAₙₛTₙₛ^{m}Cₙ;
(SEQ ID NO: 26)
AₙₛGₙₒTₙₛTₙₛGₙₛGₙₒAₙₛGₙₛ^{m}CₙₛAₙₛAₙₛGₙₛAₙₛTₙₛTₙₛAₙₛTₙₛ^{m}Cₙ;
(SEQ ID NO: 27)
AₙₛGₙₒTₙₛTₙₛGₙₛGₙₛAₙₛGₙₒ^{m}CₙₛAₙₛAₙₛGₙₛAₙₛTₙₛTₙₛAₙₛTₙₛ^{m}Cₙ;
(SEQ ID NO: 28)
AₙₛGₙₛTₙₛTₙₒGₙₒGₙₛAₙₛGₙₛ^{m}CₙₛAₙₛAₙₛGₙₛAₙₛTₙₛTₙₛAₙₛTₙₛ^{m}Cₙ;
(SEQ ID NO: 29)
AₙₛGₙₛTₙₛTₙₛGₙₛGₙₛAₙₛGₙₒ^{m}CₙₒAₙₛAₙₛGₙₛAₙₛTₙₛTₙₛAₙₛTₙₛ^{m}Cₙ;
(SEQ ID NO: 30)
AₙₛGₙₛTₙₛTₙₛGₙₛGₙₛAₙₛGₙₛ^{m}CₙₛAₙₒAₙₒGₙₛAₙₛTₙₛTₙₛAₙₛTₙₛ^{m}Cₙ;
(SEQ ID NO: 31)
AₙₛGₙₛTₙₛTₙₛGₙₛGₙₛAₙₛGₙₛ^{m}CₙₛAₙₛAₙₛGₙₒAₙₒTₙₛTₙₛAₙₛTₙₛ^{m}Cₙ;
(SEQ ID NO: 32)
AₙₛGₙₒTₙₛTₙₛGₙₛGₙₛAₙₛGₙₛ^{m}CₙₛAₙₒAₙₛGₙₛAₙₛTₙₛTₙₛAₙₛTₙₛ^{m}Cₙ;
(SEQ ID NO: 33)
AₙₛGₙₒTₙₛTₙₛGₙₛGₙₛAₙₛGₙₛ^{m}CₙₛAₙₛAₙₛGₙₒAₙₛTₙₛTₙₛAₙₛTₙₛ^{m}Cₙ;
(SEQ ID NO: 34)
AₙₛGₙₒTₙₛTₙₛGₙₛGₙₛAₙₛGₙₛ^{m}CₙₛAₙₛAₙₛGₙₛAₙₛTₙₛTₙₒAₙₛTₙₛ^{m}Cₙ;
(SEQ ID NO: 35)
AₙₛGₙₛTₙₛTₙₛGₙₛGₙₒAₙₒGₙₛ^{m}CₙₛAₙₛAₙₛGₙₛAₙₛTₙₛTₙₛAₙₛTₙₛ^{m}Cₙ;
(SEQ ID NO: 36)
AₙₛGₙₛTₙₛTₙₛGₙₛGₙₛAₙₛGₙₛ^{m}CₙₛAₙₛAₙₛGₙₛAₙₛTₙₒTₙₒAₙₛTₙₛ^{m}Cₙ;
(SEQ ID NO: 37)
AₙₛGₙₛTₙₛTₙₛGₙₛGₙₛAₙₒGₙₛ^{m}CₙₛAₙₛAₙₛGₙₛAₙₛTₙₛTₙₒAₙₛTₙₛ^{m}Cₙ;
(SEQ ID NO: 38)
AₙₛGₙₛTₙₛTₙₛGₙₛGₙₛAₙₛGₙₛ^{m}CₙₒAₙₛAₙₛGₙₛAₙₛTₙₛTₙₒAₙₛTₙₛ^{m}Cₙ;
(SEQ ID NO: 39)
AₙₛGₙₛTₙₛTₙₛGₙₛGₙₛAₙₛGₙₛ^{m}CₙₛAₙₛAₙₒGₙₛAₙₛTₙₛTₙₒAₙₛTₙₛ^{m}Cₙ;
(SEQ ID NO: 40)
AₙₛGₙₛTₙₛTₙₛGₙₛGₙₛAₙₛGₙₛ^{m}CₙₛAₙₛAₙₛGₙₛAₙₒTₙₛTₙₒAₙₛTₙₛ^{m}Cₙ;
(SEQ ID NO: 41)
AₙₛGₙₛTₙₛTₙₛGₙₛGₙₛAₙₛGₙₛ^{m}CₙₒAₙₛAₙₛGₙₛAₙₛTₙₒTₙₛAₙₛTₙₛ^{m}Cₙ;
(SEQ ID NO: 42)
AₙₛGₙₛTₙₛTₙₛGₙₛGₙₛAₙₛGₙₛ^{m}CₙₛAₙₛAₙₛGₙₒAₙₛTₙₒTₙₛAₙₛTₙₛ^{m}Cₙ;
(SEQ ID NO: 43)
AₙₒAₙₛGₙₛTₙₛTₙₛGₙₛGₙₛAₙₛGₙₛ^{m}CₙₛAₙₛAₙₛGₙₛAₙₛTₙₛTₙₛAₙₛTₙₛ^{m}Cₙ;
or
(SEQ ID NO: 44)
AₙₒAₙₛGₙₛTₙₛTₙₛGₙₛGₙₛAₙₛGₙₛ^{m}CₙₛAₙₛAₙₛGₙₛAₙₛTₙₛTₙₛAₙₛTₙₛ^{m}Cₙₒ^{m}Cₙ;

wherein:
   A=an adenine nucleobase,
   ^{m}C=a 5-methylcytosine nucleobase,
   G=a guanine nucleobase,
   T=a thymine nucleobase,
   n=a 2′-NMA sugar moiety,
   s=a phosphorothioate internucleoside linkage, and
   o=a phosphodiester internucleoside linkage.

Certain embodiments provide an oligomeric compound comprising a modified oligonucleotide according to the following chemical notation:

(SEQ ID NO: 45)
GₙₛGₙₛTₙₒAₙₒGₙₛ^{m}CₙₛAₙₛAₙₛAₙₛAₙₛGₙₛGₙₛGₙₛGₙₛTₙₛAₙₛAₙₛTₙₛAₙₛ^{m}CₙₛAₙₛGₙₛTₙ;
(SEQ ID NO: 46)
GₙₛGₙₛTₙₛAₙₛGₙₛ^{m}CₙₛAₙₛAₙₛAₙₛAₙₛGₙₛGₙₛGₙₛGₙₛTₙₛAₙₛAₙₛTₙₛAₙₛ^{m}CₙₛAₙₛGₙₛTₙ;
(SEQ ID NO: 47)
AₙₛTₙₛ^{m}Cₙₒ^{m}CₙₒAₙₛAₙₒGₙₒTₙₛTₙₒGₙₒGₙₛAₙₛGₙₛ^{m}CₙₛAₙₛAₙₛGₙₛAₙₛTₙₛTₙₛAₙₛTₙₛ^{m}Cₙₛ^{m}CₙₛTₙ;
(SEQ ID NO: 48)
Tₙₛ^{m}Cₙₛ^{m}CₙₒAₙₒAₙₛGₙₒTₙₒTₙₛGₙₒGₙₒAₙₛGₙₛ^{m}CₙₛAₙₛAₙₛGₙₛAₙₛTₙₛTₙₛAₙₛTₙₛ^{m}Cₙₛ^{m}CₙₛTₙₛAₙ;
(SEQ ID NO: 49)
^{m}Cₙₛ^{m}CₙₛAₙₒAₙₒGₙₛTₙₒTₙₒGₙₛGₙₒAₙₒGₙₛ^{m}CₙₛAₙₛAₙₛGₙₛAₙₛTₙₛTₙₛAₙₛTₙₛ^{m}Cₙₛ^{m}CₙₛTₙₛAₙₛTₙ;
(SEQ ID NO: 50)
^{m}CₙₛAₙₛAₙₒGₙₒTₙₛTₙₒGₙₒGₙₛAₙₒGₙₒ^{m}CₙₛAₙₛAₙₛGₙₛAₙₛTₙₛTₙₛAₙₛTₙₛ^{m}Cₙₛ^{m}CₙₛTₙₛAₙₛTₙₛAₙ;
(SEQ ID NO: 51)
AₙₛTₙₛ^{m}Cₙₒ^{m}CₙₒAₙₛAₙₒGₙₒTₙₛTₙₛGₙₛGₙₛAₙₛGₙₛ^{m}CₙₛAₙₛAₙₛGₙₛAₙₛTₙₛTₙₛAₙₛTₙₛ^{m}Cₙₛ^{m}CₙₛTₙ;
(SEQ ID NO: 52)
Tₙₛ^{m}Cₙₛ^{m}CₙₒAₙₒAₙₛGₙₒTₙₒTₙₛGₙₛGₙₛAₙₛGₙₛ^{m}CₙₛAₙₛAₙₛGₙₛAₙₛTₙₛTₙₛAₙₛTₙₛ^{m}Cₙₛ^{m}CₙₛTₙₛAₙ;
(SEQ ID NO: 53)
^{m}Cₙₛ^{m}CₙₛAₙₒAₙₒGₙₛTₙₒTₙₒGₙₛGₙₛAₙₛGₙₛ^{m}CₙₛAₙₛAₙₛGₙₛAₙₛTₙₛTₙₛAₙₛTₙₛ^{m}Cₙₛ^{m}CₙₛTₙₛAₙₛTₙ;
(SEQ ID NO: 54)
^{m}CₙₛAₙₛAₙₒGₙₒTₙₛTₙₒGₙₒGₙₛAₙₛGₙₛ^{m}CₙₛAₙₛAₙₛGₙₛAₙₛTₙₛTₙₛAₙₛTₙₛ^{m}Cₙₛ^{m}CₙₛTₙₛAₙₛTₙₛAₙ;
(SEQ ID NO: 55)
AₙₛTₙₛ^{m}Cₙₒ^{m}CₙₒAₙₛAₙₛGₙₛTₙₛTₙₛGₙₛGₙₛAₙₛGₙₛ^{m}CₙₛAₙₛAₙₛGₙₛAₙₛTₙₛTₙₛAₙₛTₙₛ^{m}Cₙₛ^{m}CₙₛTₙ;
(SEQ ID NO: 56)
Tₙₛ^{m}Cₙₛ^{m}CₙₒAₙₒAₙₛGₙₛTₙₛTₙₛGₙₛGₙₛAₙₛGₙₛ^{m}CₙₛAₙₛAₙₛGₙₛAₙₛTₙₛTₙₛAₙₛTₙₛ^{m}Cₙₛ^{m}CₙₛTₙₛAₙ;
(SEQ ID NO: 57)
^{m}Cₙₛ^{m}CₙₛAₙₒAₙₒGₙₛTₙₛTₙₛGₙₛGₙₛAₙₛGₙₛ^{m}CₙₛAₙₛAₙₛGₙₛAₙₛTₙₛTₙₛAₙₛTₙₛ^{m}Cₙₛ^{m}CₙₛTₙₛAₙₛTₙ;
(SEQ ID NO: 58)
^{m}CₙₛAₙₛAₙₒGₙₒTₙₛTₙₛGₙₛGₙₛAₙₛGₙₛ^{m}CₙₛAₙₛAₙₛGₙₛAₙₛTₙₛTₙₛAₙₛTₙₛ^{m}Cₙₛ^{m}CₙₛTₙₛAₙₛTₙₛAₙ;
(SEQ ID NO: 59)
AₙₛTₙₛ^{m}Cₙₛ^{m}CₙₛAₙₛAₙₛGₙₛTₙₛTₙₛGₙₛGₙₛAₙₛGₙₛ^{m}CₙₛAₙₛAₙₛGₙₛAₙₛTₙₛTₙₛAₙₛTₙₛ^{m}Cₙₛ^{m}CₙₛTₙ;
(SEQ ID NO: 60)
Tₙₛ^{m}Cₙₛ^{m}CₙₛAₙₛAₙₛGₙₛTₙₛTₙₛGₙₛGₙₛAₙₛGₙₛ^{m}CₙₛAₙₛAₙₛGₙₛAₙₛTₙₛTₙₛAₙₛTₙₛ^{m}Cₙₛ^{m}CₙₛTₙₛAₙ;
(SEQ ID NO: 61)
^{m}Cₙₛ^{m}CₙₛAₙₛAₙₛGₙₛTₙₛTₙₛGₙₛGₙₛAₙₛGₙₛ^{m}CₙₛAₙₛAₙₛGₙₛAₙₛTₙₛTₙₛAₙₛTₙₛ^{m}Cₙₛ^{m}CₙₛTₙₛAₙₛTₙ;
or
(SEQ ID NO: 62)
^{m}CₙₛAₙₛAₙₛGₙₛTₙₛTₙₛGₙₛGₙₛAₙₛGₙₛ^{m}CₙₛAₙₛAₙₛGₙₛAₙₛTₙₛTₙₛAₙₛTₙₛ^{m}Cₙₛ^{m}CₙₛTₙₛAₙₛTₙₛAₙ,

wherein:
   A=an adenine nucleobase,
   ^{m}C=a 5-methylcytosine nucleobase,
   G=a guanine nucleobase,
   T=a thymine nucleobase,
   n=a 2′-NMA sugar moiety,
   s=a phosphorothioate internucleoside linkage, and
   o=a phosphodiester internucleoside linkage.

### A. Certain Modified Nucleosides

Modified nucleosides comprise a modified sugar moiety or a modified nucleobase or both a modified sugar moiety and a modified nucleobase. In certain embodiments, modified nucleosides comprising the following modified sugar moieties and/or the following modified nucleobases may be incorporated into modified oligonucleotides.

### 1. Certain Sugar Moieties

In certain embodiments, modified sugar moieties are non-bicyclic modified sugar moieties comprising a furanosyl ring with one or more substituent groups none of which bridges two atoms of the furanosyl ring to form a bicyclic structure. Such non bridging substituents may be at any position of the furanosyl, including but not limited to substituents at the 2′, 3′, 4′, and/or 5′ positions. Examples of 2′-substituent groups suitable for non-bicyclic modified sugar moieties include but are not limited to: 2′-O(CH₂)₂OCH₃ (“MOE” or “O-methoxyethyl”), and 2′-O—N-methyl acetamide (“NMA”) (see U.S. Pat. No. 6,147,200, Prakash et al., 2003, Org. Lett., 5, 403-6).

A “2′-O—N-methyl acetamide nucleoside” or “2′-NMA nucleoside” is shown below:

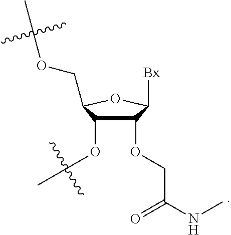

In certain embodiments, the non-bicyclic modified sugar moiety is a 2′-MOE sugar moiety or a 2′-NMA sugar moiety. In certain embodiments, each nucleoside of the modified oligonucleotide comprises a modified sugar moiety. In certain embodiments the modified sugar moiety is a 2′-NMA sugar moiety. In certain embodiments, each nucleoside of the modified oligonucleotide comprises a 2′-NMA sugar moiety.

In certain embodiments, modified furanosyl sugar moieties and nucleosides incorporating such modified furanosyl sugar moieties are further defined by isomeric configuration. For example, a 2′-deoxyfuranosyl sugar moiety may be in seven isomeric configurations other than the naturally occurring β-D-deoxyribosyl configuration. Such modified sugar moieties are described in, e.g., WO 2019/157531, incorporated by reference herein. A 2′-modified sugar moiety has an additional stereocenter at the 2′-position relative to a 2′-deoxyfuranosyl sugar moiety; therefore, such sugar moieties have a total of sixteen possible isomeric configurations. 2′-modified sugar moieties described herein are in the β-D-ribosyl isomeric configuration unless otherwise specified.

### 2. Certain Modified Nucleobases

In certain embodiments, modified oligonucleotides comprise one or more nucleosides comprising an unmodified nucleobase. In certain embodiments, modified oligonucleotides comprise one or more nucleosides comprising a modified nucleobase. Examples of modified nucleobases include 5-methylcytosine.

Publications that teach the preparation of certain modified nucleobases include without limitation, Manoharan et al., US2003/0158403; Manoharan et al., US2003/0175906; Dinh et al., U.S. Pat. No. 4,845,205; Spielvogel et al., U.S. Pat. No. 5,130,302; Rogers et al., U.S. Pat. No. 5,134,066; Bischofberger et al., U.S. Pat. No. 5,175,273; Urdea et al., U.S. Pat. No. 5,367,066; Benner et al., U.S. Pat. No. 5,432,272; Matteucci et al., U.S. Pat. No. 5,434,257; Gmeiner et al., U.S. Pat. No. 5,457,187; Cook et al., U.S. Pat. No. 5,459,255; Froehler et al., U.S. Pat. No. 5,484,908; Matteucci et al., U.S. Pat. No. 5,502,177; Hawkins et al., U.S. Pat. No. 5,525,711; Haralambidis et al., U.S. Pat. No. 5,552,540; Cook et al., U.S. Pat. No. 5,587,469; Froehler et al., U.S. Pat. No. 5,594,121; Switzer et al., U.S. Pat. No. 5,596,091; Cook et al., U.S. Pat. No. 5,614,617; Froehler et al., U.S. Pat. No. 5,645,985; Cook et al., U.S. Pat. No. 5,681,941; Cook et al., U.S. Pat. No. 5,811,534; Cook et al., U.S. Pat. No. 5,750,692; Cook et al., U.S. Pat. No. 5,948,903; Cook et al., U.S. Pat. No. 5,587,470; Cook et al., U.S. Pat. No. 5,457,191; Matteucci et al., U.S. Pat. No. 5,763,588; Froehler et al., U.S. Pat. No. 5,830,653; Cook et al., U.S. Pat. No. 5,808,027; Cook et al., U.S. Pat. No. 6,166,199; and Matteucci et al., U.S. Pat. No. 6,005,096.

### 3. Certain Modified Internucleoside Linkages

The naturally occurring internucleoside linkage of RNA and DNA is a 3′ to 5′ phosphodiester linkage. In certain embodiments, nucleosides of modified oligonucleotides may be linked together using one or more modified internucleoside linkages. The two main classes of internucleoside linking groups are defined by the presence or absence of a phosphorus atom. Representative phosphorus-containing internucleoside linkages include but are not limited to phosphates, which contain a phosphodiester bond (“P═O”) (also referred to as unmodified or naturally occurring linkages), phosphotriesters, methylphosphonates, phosphoramidates, and phosphorothioates (“P═S”), and phosphorodithioates (“HS—P═S”). Modified internucleoside linkages, compared to naturally occurring phosphate linkages, can be used to alter, typically increase, nuclease resistance of the oligonucleotide. In certain embodiments, internucleoside linkages having a chiral atom can be prepared as a racemic mixture, or as separate enantiomers. Methods of preparation of phosphorous-containing and non-phosphorous-containing internucleoside linkages are well known to those skilled in the art.

Representative internucleoside linkages having a chiral center include but are not limited to phosphorothioates. Modified oligonucleotides comprising internucleoside linkages having a chiral center can be prepared as populations of modified oligonucleotides comprising stereorandom internucleoside linkages, or as populations of modified oligonucleotides comprising phosphorothioate or other linkages containing chiral centers in particular stereochemical configurations. In certain embodiments, populations of modified oligonucleotides comprise phosphorothioate internucleoside linkages wherein all of the phosphorothioate internucleoside linkages are stereorandom. Such modified oligonucleotides can be generated using synthetic methods that result in random selection of the stereochemical configuration of each phosphorothioate linkage. Nonetheless, each individual phosphorothioate of each individual oligonucleotide molecule has a defined stereoconfiguration. In certain embodiments, populations of modified oligonucleotides are enriched for modified oligonucleotides comprising one or more particular phosphorothioate internucleoside linkages in a particular, independently selected stereochemical configuration. In certain embodiments, the particular configuration of the particular phosphorothioate linkage is present in at least 65% of the molecules in the population. In certain embodiments, the particular configuration of the particular phosphorothioate linkage is present in at least 70% of the molecules in the population. In certain embodiments, the particular configuration of the particular phosphorothioate linkage is present in at least 80% of the molecules in the population. In certain embodiments, the particular configuration of the particular phosphorothioate linkage is present in at least 90% of the molecules in the population. In certain embodiments, the particular configuration of the particular phosphorothioate linkage is present in at least 99% of the molecules in the population. Such chirally enriched populations of modified oligonucleotides can be generated using synthetic methods known in the art, e.g., methods described in Oka et al., JACS 125, 8307 (2003), Wan et al. Nuc. Acid. Res. 42, 13456 (2014), and WO 2017/015555. In certain embodiments, a population of modified oligonucleotides is enriched for modified oligonucleotides having at least one indicated phosphorothioate in the (Sp) configuration. In certain embodiments, a population of modified oligonucleotides is enriched for modified oligonucleotides having at least one phosphorothioate in the (Rp) configuration. In certain embodiments, modified oligonucleotides comprising (Rp) and/or (Sp) phosphorothioates comprise one or more of the following formulas, respectively, wherein “B” indicates a nucleobase:

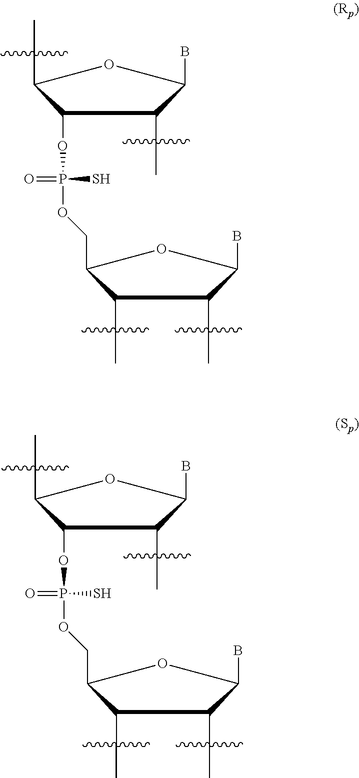

Unless otherwise indicated, chiral internucleoside linkages of modified oligonucleotides described herein can be stereorandom or in a particular stereochemical configuration.

### B. Certain Motifs

In certain embodiments, modified oligonucleotides comprise one or more modified nucleosides comprising a modified sugar moiety. In certain embodiments, modified oligonucleotides comprise one or more modified nucleosides comprising a modified nucleobase. In certain embodiments, modified oligonucleotides comprise one or more modified internucleoside linkage. In such embodiments, the modified, unmodified, and differently modified sugar moieties, nucleobases, and/or internucleoside linkages of a modified oligonucleotide define a pattern or motif. In certain embodiments, the patterns of sugar moieties, nucleobases, and internucleoside linkages are each independent of one another. Thus, a modified oligonucleotide may be described by its sugar motif, nucleobase motif and/or internucleoside linkage motif (as used herein, nucleobase motif describes the modifications to the nucleobases independent of the sequence of nucleobases).

### 1. Certain Sugar Motifs

In certain embodiments, oligonucleotides comprise one or more type of modified sugar and/or unmodified sugar moiety arranged along the oligonucleotide or region thereof in a defined pattern or sugar motif. In certain instances, such sugar motifs include but are not limited to any of the sugar modifications discussed herein.

In certain embodiments, each nucleoside of a modified oligonucleotide, or portion thereof, comprises a 2′-substituted sugar moiety, a bicyclic sugar moiety, a sugar surrogate, or a 2′-deoxyribosyl sugar moiety. In certain embodiments, the 2′-substituted sugar moiety is selected from a 2′-MOE sugar moiety or a 2′-NMA sugar moiety.

In certain embodiments, each nucleoside of a modified oligonucleotide comprises a modified sugar moiety (“fully modified oligonucleotide”). In certain embodiments, each nucleoside of a fully modified oligonucleotide comprises a 2′-substituted sugar moiety. In certain embodiments, the 2′-substituted sugar moiety is selected from a 2′-MOE sugar moiety or a 2′-NMA sugar moiety. In certain embodiments, each nucleoside of a fully modified oligonucleotide comprises the same modified sugar moiety (“uniformly modified sugar motif”). In certain embodiments, the uniformly modified sugar motif is 7 to 20 nucleosides in length. In certain embodiments, each nucleoside of the uniformly modified sugar motif comprises a 2′-substituted sugar moiety. In certain embodiments, the 2′-substituted sugar moiety is selected from a 2′-MOE sugar moiety or a 2′-NMA sugar moiety. In certain embodiments, the 2′-substituted sugar moiety is a 2′-NMA sugar moiety.

### 2. Certain Nucleobase Motifs

In certain embodiments, oligonucleotides comprise modified and/or unmodified nucleobases arranged along the oligonucleotide or region thereof in a defined pattern or motif. In certain embodiments, each nucleobase is modified. In certain embodiments, none of the nucleobases are modified. In certain embodiments, each purine or each pyrimidine is modified. In certain embodiments, each cytosine is modified. In certain embodiments, some, or all of the cytosine nucleobases in a modified oligonucleotide are 5-methylcytosines. In certain embodiments, all of the cytosine nucleobases are 5-methylcytosines and all of the other nucleobases of the modified oligonucleotide are unmodified nucleobases.

### 3. Certain Internucleoside Linkage Motifs

In certain embodiments, oligonucleotides comprise modified and/or unmodified internucleoside linkages arranged along the oligonucleotide or region thereof in a defined pattern or motif. In certain embodiments, each internucleoside linking group is a phosphodiester internucleoside linkage (P═O). In certain embodiments, each internucleoside linking group of a modified oligonucleotide is a phosphorothioate internucleoside linkage (P═S). In certain embodiments, each internucleoside linkage of a modified oligonucleotide is independently selected from a phosphorothioate internucleoside linkage and phosphodiester internucleoside linkage. In certain embodiments, each phosphorothioate internucleoside linkage is independently selected from a stereorandom phosphorothioate, a (Sp) phosphorothioate, and a (Rp) phosphorothioate.

In certain embodiments, modified oligonucleotides have an internucleoside linkage motif of (5′ to 3′): soossssssssssssss, wherein each “s” represents a phosphorothioate internucleoside linkage and each “o” represents a phosphodiester internucleoside linkage. In certain embodiments, modified oligonucleotides have an internucleoside linkage motif of (5′ to 3′): sososssssssssssss, wherein each “s” represents a phosphorothioate internucleoside linkage and each “o” represents a phosphodiester internucleoside linkage. In certain embodiments, modified oligonucleotides have an internucleoside linkage motif of (5′ to 3′): sossossssssssssss, wherein each “s” represents a phosphorothioate internucleoside linkage and each “o” represents a phosphodiester internucleoside linkage. In certain embodiments, modified oligonucleotides have an internucleoside linkage motif of (5′ to 3′): sosssosssssssssss, wherein each “s” represents a phosphorothioate internucleoside linkage and each “o” represents a phosphodiester internucleoside linkage. In certain embodiments, modified oligonucleotides have an internucleoside linkage motif of (5′ to 3′): sosssssosssssssss, wherein each “s” represents a phosphorothioate internucleoside linkage and each “o” represents a phosphodiester internucleoside linkage. In certain embodiments, modified oligonucleotides have an internucleoside linkage motif of (5′ to 3′): sssoossssssssssss, wherein each “s” represents a phosphorothioate internucleoside linkage and each “o” represents a phosphodiester internucleoside linkage. In certain embodiments, modified oligonucleotides have an internucleoside linkage motif of (5′ to 3′): sssssssoossssssss, wherein each “s” represents a phosphorothioate internucleoside linkage and each “o” represents a phosphodiester internucleoside linkage. In certain embodiments, modified oligonucleotides have an internucleoside linkage motif of (5′ to 3′): sssssssssoossssss, wherein each “s” represents a phosphorothioate internucleoside linkage and each “o” represents a phosphodiester internucleoside linkage. In certain embodiments, modified oligonucleotides have an internucleoside linkage motif of (5′ to 3′): sssssssssssoossss, wherein each “s” represents a phosphorothioate internucleoside linkage and each “o” represents a phosphodiester internucleoside linkage. In certain embodiments, modified oligonucleotides have an internucleoside linkage motif of (5′ to 3′): sosssssssosssssss, wherein each “s” represents a phosphorothioate internucleoside linkage and each “o” represents a phosphodiester internucleoside linkage. In certain embodiments, modified oligonucleotides have an internucleoside linkage motif of (5′ to 3′): sosssssssssosssss, wherein each “s” represents a phosphorothioate internucleoside linkage and each “o” represents a phosphodiester internucleoside linkage. In certain embodiments, modified oligonucleotides have an internucleoside linkage motif of (5′ to 3′): sossssssssssssoss, wherein each “s” represents a phosphorothioate internucleoside linkage and each “o” represents a phosphodiester internucleoside linkage. In certain embodiments, modified oligonucleotides have an internucleoside linkage motif of (5′ to 3′): sssssoossssssssss, wherein each “s” represents a phosphorothioate internucleoside linkage and each “o” represents a phosphodiester internucleoside linkage. In certain embodiments, modified oligonucleotides have an internucleoside linkage motif of (5′ to 3′): sssssssssssssooss, wherein each “s” represents a phosphorothioate internucleoside linkage and each “o” represents a phosphodiester internucleoside linkage. In certain embodiments, modified oligonucleotides have an internucleoside linkage motif of (5′ to 3′): ssssssosssssssoss, wherein each “s” represents a phosphorothioate internucleoside linkage and each “o” represents a phosphodiester internucleoside linkage. In certain embodiments, modified oligonucleotides have an internucleoside linkage motif of (5′ to 3′): ssssssssosssssoss, wherein each “s” represents a phosphorothioate internucleoside linkage and each “o” represents a phosphodiester internucleoside linkage. In certain embodiments, modified oligonucleotides have an internucleoside linkage motif of (5′ to 3′): ssssssssssosssoss, wherein each “s” represents a phosphorothioate internucleoside linkage and each “o” represents a phosphodiester internucleoside linkage. In certain embodiments, modified oligonucleotides have an internucleoside linkage motif of (5′ to 3′): ssssssssssssososs, wherein each “s” represents a phosphorothioate internucleoside linkage and each “o” represents a phosphodiester internucleoside linkage. In certain embodiments, modified oligonucleotides have an internucleoside linkage motif of (5′ to 3′): ssssssssossssosss, wherein each “s” represents a phosphorothioate internucleoside linkage and each “o” represents a phosphodiester internucleoside linkage. In certain embodiments, modified oligonucleotides have an internucleoside linkage motif of (5′ to 3′): sssssssssssososss, wherein each “s” represents a phosphorothioate internucleoside linkage and each “o” represents a phosphodiester internucleoside linkage. In certain embodiments, modified oligonucleotides have an internucleoside linkage motif of (5′ to 3′): osssssssssssssssss, wherein each “s” represents a phosphorothioate internucleoside linkage and each “o” represents a phosphodiester internucleoside linkage. In certain embodiments, modified oligonucleotides have an internucleoside linkage motif of (5′ to 3′): ossssssssssssssssso, wherein each “s” represents a phosphorothioate internucleoside linkage and each “o” represents a phosphodiester internucleoside linkage. In certain embodiments, modified oligonucleotides have an internucleoside linkage motif of (5′ to 3′): ssoosoosoossssssssssssss, wherein each “s” represents a phosphorothioate internucleoside linkage and each “o” represents a phosphodiester internucleoside linkage. In certain embodiments, modified oligonucleotides have an internucleoside linkage motif of (5′ to 3′): ssoossssssssssssssssssss, wherein each “s” represents a phosphorothioate internucleoside linkage and each “o” represents a phosphodiester internucleoside linkage. In certain embodiments, modified oligonucleotides have an internucleoside linkage motif of (5′ to 3′): ssoossssssssssssssssss, wherein each “s” represents a phosphorothioate internucleoside linkage and each “o” represents a phosphodiester internucleoside linkage. In certain embodiments, modified oligonucleotides have an internucleoside linkage motif of (5′ to 3′): ssoosoosssssssssssssssss, wherein each “s” represents a phosphorothioate internucleoside linkage and each “o” represents a phosphodiester internucleoside linkage.

### 4. Certain Lengths

In certain embodiments, oligonucleotides (including modified oligonucleotides) can have any of a variety of ranges of lengths. In certain embodiments, oligonucleotides consist of X to Y linked nucleosides, where X represents the fewest number of nucleosides in the range and Y represents the largest number nucleosides in the range. In certain such embodiments, X and Y are each independently selected from 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50; provided that X<Y. For example, in certain embodiments, oligonucleotides consist of 17 to 18, 17 to 19, 17 to 20, 17 to 21, 17 to 22, 17 to 23, 17 to 24, 17 to 25, 17 to 26, 17 to 27, 17 to 28, 17 to 29, 17 to 30, 18 to 19, 18 to 20, 18 to 21, 18 to 22, 18 to 23, 18 to 24, 18 to 25, 18 to 26, 18 to 27, 18 to 28, 18 to 29, 18 to 30, 19 to 20, 19 to 21, 19 to 22, 19 to 23, 19 to 24, 19 to 25, 19 to 26, 19 to 27, 19 to 28, 19 to 29, 19 to 30, 20 to 21, 20 to 22, 20 to 23, 20 to 24, 20 to 25, 20 to 26, 20 to 27, 20 to 28, 20 to 29, 20 to 30, 21 to 22, 21 to 23, 21 to 24, 21 to 25, 21 to 26, 21 to 27, 21 to 28, 21 to 29, 21 to 30, 22 to 23, 22 to 24, 22 to 25, 22 to 26, 22 to 27, 22 to 28, 22 to 29, 22 to 30, 23 to 24, 23 to 25, 23 to 26, 23 to 27, 23 to 28, 23 to 29, 23 to 30, 24 to 25, 24 to 26, 24 to 27, 24 to 28, 24 to 29, 24 to 30, 25 to 26, 25 to 27, 25 to 28, 25 to 29, 25 to 30, 26 to 27, 26 to 28, 26 to 29, 26 to 30, 27 to 28, 27 to 29, 27 to 30, 28 to 29, 28 to 30, or 29 to 30 linked nucleosides.

In certain embodiments, oligonucleotides consist of 17 linked nucleosides. In certain embodiments, oligonucleotides consist of 18 linked nucleosides. In certain embodiments, oligonucleotides consist of 19 linked nucleosides. In certain embodiments, oligonucleotides consist of 20 linked nucleosides. In certain embodiments, oligonucleotides consist of 23 linked nucleosides. In certain embodiments, oligonucleotides consist of 25 linked nucleosides.

### B. Certain Populations of Modified Oligonucleotides

Populations of modified oligonucleotides in which all of the modified oligonucleotides of the population have the same molecular formula can be stereorandom populations or chirally enriched populations. All of the chiral centers of all of the modified oligonucleotides are stereorandom in a stereorandom population. In a chirally enriched population, at least one particular chiral center is not stereorandom in the modified oligonucleotides of the population.

### Certain Oligomeric Compounds

In certain embodiments, provided herein are oligomeric compounds, which consist of an oligonucleotide (modified or unmodified) and optionally one or more conjugate groups and/or terminal groups. Conjugate groups consist of one or more conjugate moiety and a conjugate linker which links the conjugate moiety to the oligonucleotide. Conjugate groups may be attached to either or both ends of an oligonucleotide and/or at any internal position. In certain embodiments, conjugate groups are attached to the 2′-position of a nucleoside of a modified oligonucleotide. In certain embodiments, conjugate groups that are attached to either or both ends of an oligonucleotide are terminal groups. In certain such embodiments, conjugate groups or terminal groups are attached at the 3′ and/or 5′-end of oligonucleotides. In certain such embodiments, conjugate groups (or terminal groups) are attached at the 3′-end of oligonucleotides. In certain embodiments, conjugate groups are attached near the 3′-end of oligonucleotides. In certain embodiments, conjugate groups (or terminal groups) are attached at the 5′-end of oligonucleotides. In certain embodiments, conjugate groups are attached near the 5′-end of oligonucleotides.

Examples of terminal groups include but are not limited to conjugate groups, capping groups, phosphate moieties, protecting groups, modified or unmodified nucleosides, and two or more nucleosides that are independently modified or unmodified.

### A. Certain Conjugate Groups

In certain embodiments, oligonucleotides are covalently attached to one or more conjugate groups. In certain embodiments, conjugate groups modify one or more properties of the attached oligonucleotide, including but not limited to pharmacodynamics, pharmacokinetics, stability, binding, absorption, tissue distribution, cellular distribution, cellular uptake, charge and clearance.

In certain embodiments, conjugation of one or more carbohydrate moieties to a modified oligonucleotide can optimize one or more properties of the modified oligonucleotide. In certain embodiments, the carbohydrate moiety is attached to a modified subunit of the modified oligonucleotide. For example, the ribose sugar of one or more ribonucleotide subunits of a modified oligonucleotide can be replaced with another moiety, e.g., a non-carbohydrate (preferably cyclic) carrier to which is attached a carbohydrate ligand. A ribonucleotide subunit in which the ribose sugar of the subunit has been so replaced is referred to herein as a ribose replacement modification subunit (RRMS), which is a modified sugar moiety. A cyclic carrier may be a carbocyclic ring system, i.e., one or more ring atoms may be a heteroatom, e.g., nitrogen, oxygen, sulphur. The cyclic carrier may be a monocyclic ring system, or may contain two or more rings, e.g., fused rings. The cyclic carrier may be a fully saturated ring system, or it may contain one or more double bonds.

In certain embodiments, conjugate groups impart a new property on the attached oligonucleotide, e.g., fluorophores or reporter groups that enable detection of the oligonucleotide. Certain conjugate groups and conjugate moieties have been described previously, for example: cholesterol moiety (Letsinger et al., Proc. Natl. Acad. Sci. USA, 1989, 86, 6553-6556), cholic acid (Manoharan et al., Bioorg. Med. Chem. Lett., 1994, 4, 1053-1060), a thioether, e.g., hexyl-S-tritylthiol (Manoharan et al., Ann. N. Y. Acad. Sci., 1992, 660, 306-309; Manoharan et al., Bioorg. Med. Chem. Lett., 1993, 3, 2765-2770), a thiocholesterol (Oberhauser et al., Nucl. Acids Res., 1992, 20, 533-538), an aliphatic chain, e.g., do-decan-diol or undecyl residues (Saison-Behmoaras et al., EIMBO J, 1991, 10, 1111-1118; Kabanov et al., FEBS Lett., 1990, 259, 327-330; Svinarchuk et al., Biochimie, 1993, 75, 49-54), a phospholipid, e.g., di-hexadecyl-rac-glycerol or triethyl-ammonium 1,2-di-O-hexadecyl-rac-glycero-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Manoharan et al., Nucleosides & Nucleotides, 1995, 14, 969-973), or adamantane acetic acid a palmityl moiety (Mishra et al., Biochim. Biophys. Acta, 1995, 1264, 229-237), an octadecylamine or hexylamino-carbonyl-oxycholesterol moiety (Crooke et al., J. Pharmacol. Exp. Ther., 1996, 277, 923-937), a tocopherol group (Nishina et al., Molecular Therapy Nucleic Acids, 2015, 4, e220; and Nishina et al., Molecular Therapy, 2008, 16, 734-740), or a GalNAc cluster (e.g., WO2014/179620).

In certain embodiments, the conjugate group may comprise a conjugate moiety selected from any of a C22 alkyl, C20 alkyl, C16 alkyl, C10 alkyl, C21 alkyl, C19 alkyl, C18 alkyl, C17 alkyl, C15 alkyl, C14 alkyl, C13 alkyl, C12 alkyl, C11 alkyl, C9 alkyl, C8 alkyl, C7 alkyl, C6 alkyl, C5 alkyl, C22 alkenyl, C20 alkenyl, C16 alkenyl, C10 alkenyl, C21 alkenyl, C19 alkenyl, C18 alkenyl, C17 alkenyl, C15 alkenyl, C14 alkenyl, C13 alkenyl, C12 alkenyl, C11 alkenyl, C9 alkenyl, C8 alkenyl, C7 alkenyl, C6 alkenyl, or C5 alkenyl.

In certain embodiments, the conjugate group may comprise a conjugate moiety selected from any of a C22 alkyl, C20 alkyl, C16 alkyl, C10 alkyl, C21 alkyl, C19 alkyl, C18 alkyl, C17 alkyl, C15 alkyl, C14 alkyl, C13 alkyl, C12 alkyl, C11 alkyl, C9 alkyl, C8 alkyl, C7 alkyl, C6 alkyl, or C5 alkyl, where the alkyl chain has one or more unsaturated bonds.

In certain embodiments, a conjugate group is a lipid having the following structure:

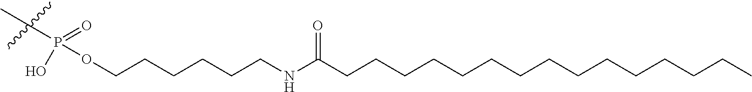 [SMILES: *P(=O)(O)OCCCCCCNC(=O)CCCCCCCCCCCCCCC]

### 1. Conjugate Moieties

Conjugate moieties include, without limitation, intercalators, reporter molecules, polyamines, polyamides, peptides, carbohydrates (e.g., GalNAc), vitamin moieties, polyethylene glycols, thioethers, polyethers, cholesterols, thiocholesterols, cholic acid moieties, folate, lipids, phospholipids, biotin, phenazine, phenanthridine, anthraquinone, adamantane, acridine, fluoresceins, rhodamines, coumarins, fluorophores, and dyes.

In certain embodiments, a conjugate moiety comprises an active drug substance, for example, aspirin, warfarin, phenylbutazone, ibuprofen, suprofen, fen-bufen, ketoprofen, (S)-(+)-pranoprofen, carprofen, dansylsarcosine, 2,3,5-triiodobenzoic acid, fingolimod, flufenamic acid, folinic acid, a benzothiadiazide, chlorothiazide, a diazepine, indo-methicin, a barbiturate, a cephalosporin, a sulfa drug, an antidiabetic, an antibacterial or an antibiotic.

### 2. Conjugate Linkers

Conjugate moieties are attached to oligonucleotides through conjugate linkers. In certain oligomeric compounds, the conjugate linker is a single chemical bond (i.e., the conjugate moiety is attached directly to an oligonucleotide through a single bond). In certain embodiments, the conjugate linker comprises a chain structure, such as a hydrocarbyl chain, or an oligomer of repeating units such as ethylene glycol, nucleosides, or amino acid units.

In certain embodiments, a conjugate linker comprises pyrrolidine.

In certain embodiments, a conjugate linker comprises one or more groups selected from alkyl, amino, oxo, amide, disulfide, polyethylene glycol, ether, thioether, and hydroxylamino. In certain such embodiments, the conjugate linker comprises groups selected from alkyl, amino, oxo, amide and ether groups. In certain embodiments, the conjugate linker comprises groups selected from alkyl and amide groups. In certain embodiments, the conjugate linker comprises groups selected from alkyl and ether groups. In certain embodiments, the conjugate linker comprises at least one phosphorus moiety. In certain embodiments, the conjugate linker comprises at least one phosphate group. In certain embodiments, the conjugate linker includes at least one neutral linking group.

In certain embodiments, conjugate linkers, including the conjugate linkers described above, are bifunctional linking moieties, e.g., those known in the art to be useful for attaching conjugate moieties to compounds, such as the oligonucleotides provided herein. In general, a bifunctional linking moiety comprises at least two functional groups. One of the functional groups is selected to react with a particular site on a compound and the other is selected to react with a conjugate moiety. Examples of functional groups used in a bifunctional linking moiety include but are not limited to electrophiles for reacting with nucleophilic groups and nucleophiles for reacting with electrophilic groups. In certain embodiments, bifunctional linking moieties comprise one or more groups selected from amino, hydroxyl, carboxylic acid, thiol, alkyl, alkenyl, and alkynyl.

Examples of conjugate linkers include but are not limited to pyrrolidine, 8-amino-3,6-dioxaoctanoic acid (ADO), succinimidyl 4-(N-maleimidomethyl) cyclohexane-1-carboxylate (SMCC) and 6-aminohexanoic acid (AHEX or AHA). Other conjugate linkers include but are not limited to substituted or unsubstituted C₁-C₁₀ alkyl, substituted or unsubstituted C₂-C₁₀ alkenyl or substituted or unsubstituted C₂-C₁₀ alkynyl, wherein a nonlimiting list of preferred substituent groups includes hydroxyl, amino, alkoxy, carboxy, benzyl, phenyl, nitro, thiol, thioalkoxy, halogen, alkyl, aryl, alkenyl and alkynyl.

In certain embodiments, conjugate linkers comprise 1-10 linker-nucleosides. In certain embodiments, conjugate linkers comprise 2-5 linker-nucleosides. In certain embodiments, conjugate linkers comprise exactly 3 linker-nucleosides. In certain embodiments, conjugate linkers comprise the TCA motif. In certain embodiments, such linker-nucleosides are modified nucleosides. In certain embodiments such linker-nucleosides comprise a modified sugar moiety. In certain embodiments, linker-nucleosides are unmodified. In certain embodiments, linker-nucleosides comprise an optionally protected heterocyclic base selected from a purine, substituted purine, pyrimidine or substituted pyrimidine. In certain embodiments, a cleavable moiety is a nucleoside selected from uracil, thymine, cytosine, 4-N-benzoylcytosine, 5-methylcytosine, 4-N-benzoyl-5-methylcytosine, adenine, 6-N-benzoyladenine, guanine and 2-N-isobutyrylguanine. It is typically desirable for linker-nucleosides to be cleaved from the oligomeric compound after it reaches a target tissue. Accordingly, linker-nucleosides are typically linked to one another and to the remainder of the oligomeric compound through cleavable bonds. In certain embodiments, such cleavable bonds are phosphodiester bonds.

Herein, linker-nucleosides are not considered to be part of the oligonucleotide. Accordingly, in embodiments in which an oligomeric compound comprises an oligonucleotide consisting of a specified number or range of linked nucleosides and/or a specified percent complementarity to a reference nucleic acid and the oligomeric compound also comprises a conjugate group comprising a conjugate linker comprising linker-nucleosides, those linker-nucleosides are not counted toward the length of the oligonucleotide and are not used in determining the percent complementarity of the oligonucleotide for the reference nucleic acid. For example, an oligomeric compound may comprise (1) a modified oligonucleotide consisting of 8-30 nucleosides and (2) a conjugate group comprising 1-10 linker-nucleosides that are contiguous with the nucleosides of the modified oligonucleotide. The total number of contiguous linked nucleosides in such an oligomeric compound is more than 30. Alternatively, an oligomeric compound may comprise a modified oligonucleotide consisting of 8-30 nucleosides and no conjugate group. The total number of contiguous linked nucleosides in such an oligomeric compound is no more than 30. Unless otherwise indicated conjugate linkers comprise no more than 10 linker-nucleosides. In certain embodiments, conjugate linkers comprise no more than 5 linker-nucleosides. In certain embodiments, conjugate linkers comprise no more than 3 linker-nucleosides. In certain embodiments, conjugate linkers comprise no more than 2 linker-nucleosides. In certain embodiments, conjugate linkers comprise no more than 1 linker-nucleoside.

In certain embodiments, it is desirable for a conjugate group to be cleaved from the oligonucleotide. For example, in certain circumstances oligomeric compounds comprising a particular conjugate moiety are better taken up by a particular cell type, but once the oligomeric compound has been taken up, it is desirable that the conjugate group be cleaved to release the unconjugated or parent oligonucleotide. Thus, certain conjugate linkers may comprise one or more cleavable moieties. In certain embodiments, a cleavable moiety is a cleavable bond. In certain embodiments, a cleavable moiety is a group of atoms comprising at least one cleavable bond. In certain embodiments, a cleavable moiety comprises a group of atoms having one, two, three, four, or more than four cleavable bonds. In certain embodiments, a cleavable moiety is selectively cleaved inside a cell or subcellular compartment, such as a lysosome. In certain embodiments, a cleavable moiety is selectively cleaved by endogenous enzymes, such as nucleases.

In certain embodiments, a cleavable bond is selected from among an amide, an ester, an ether, one or both esters of a phosphodiester, a phosphate ester, a carbamate, or a disulfide. In certain embodiments, a cleavable bond is one or both of the esters of a phosphodiester. In certain embodiments, a cleavable moiety comprises a phosphate or phosphodiester. In certain embodiments, the cleavable moiety is a phosphate linkage between an oligonucleotide and a conjugate moiety or conjugate group.

In certain embodiments, a cleavable moiety comprises or consists of one or more linker-nucleosides. In certain such embodiments, the one or more linker-nucleosides are linked to one another and/or to the remainder of the oligomeric compound through cleavable bonds. In certain embodiments, such cleavable bonds are unmodified phosphodiester bonds. In certain embodiments, a cleavable moiety is 2′-deoxynucleoside that is attached to either the 3′ or 5′-terminal nucleoside of an oligonucleotide by a phosphate internucleoside linkage and covalently attached to the remainder of the conjugate linker or conjugate moiety by a phosphate or phosphorothioate linkage. In certain such embodiments, the cleavable moiety is 2′-deoxyadenosine.

### 3. Cell-Targeting Moieties

In certain embodiments, a conjugate group comprises a cell-targeting moiety. In certain embodiments, a conjugate group has the general formula:
wherein n is from 1 to about 3, m is 0 when n is 1, m is 1 when n is 2 or greater, j is 1 or 0, and k is 1 or 0.

In certain embodiments, n is 1, j is 1 and k is 0. In certain embodiments, n is 1, j is 0 and k is 1. In certain embodiments, n is 1, j is 1 and k is 1. In certain embodiments, n is 2, j is 1 and k is 0. In certain embodiments, n is 2, j is 0 and k is 1. In certain embodiments, n is 2, j is 1 and k is 1. In certain embodiments, n is 3, j is 1 and k is 0. In certain embodiments, n is 3, j is 0 and k is 1. In certain embodiments, n is 3, j is 1 and k is 1.

In certain embodiments, conjugate groups comprise cell-targeting moieties that have at least one tethered ligand. In certain embodiments, cell-targeting moieties comprise two tethered ligands covalently attached to a branching group.

In certain embodiments, each ligand of a cell-targeting moiety has an affinity for at least one type of receptor on a target cell. In certain embodiments, each ligand has an affinity for at least one type of receptor on the surface of a mammalian liver cell. In certain embodiments, each ligand has an affinity for the hepatic asialoglycoprotein receptor (ASGP-R). In certain embodiments, each ligand is a carbohydrate.

In certain embodiments, a conjugate group comprises a cell-targeting conjugate moiety. In certain embodiments, a conjugate group has the general formula:
wherein n is from 1 to about 3, m is 0 when n is 1, m is 1 when n is 2 or greater, j is 1 or 0, and k is 1 or 0.

In certain embodiments, n is 1, j is 1 and k is 0. In certain embodiments, n is 1, j is 0 and k is 1. In certain embodiments, n is 1, j is 1 and k is 1. In certain embodiments, n is 2, j is 1 and k is 0. In certain embodiments, n is 2, j is 0 and k is 1. In certain embodiments, n is 2, j is 1 and k is 1. In certain embodiments, n is 3, j is 1 and k is 0. In certain embodiments, n is 3, j is 0 and k is 1. In certain embodiments, n is 3, j is 1 and k is 1.

In certain embodiments, conjugate groups comprise cell-targeting moieties that have at least one tethered ligand. In certain embodiments, cell-targeting moieties comprise two tethered ligands covalently attached to a branching group. In certain embodiments, cell-targeting moieties comprise three tethered ligands covalently attached to a branching group.

### B. Certain Terminal Groups

In certain embodiments, oligomeric compounds comprise one or more terminal groups. In certain such embodiments, oligomeric compounds comprise a stabilized 5′-phosphate. Stabilized 5′-phosphates include, but are not limited to 5′-phosphonates, including, but not limited to 5′-vinylphosphonates. In certain embodiments, terminal groups comprise one or more abasic sugar moieties and/or inverted nucleosides. In certain embodiments, terminal groups comprise one or more 2′-linked nucleosides or sugar moieties. In certain such embodiments, the 2′-linked group is an abasic sugar moiety.

### I. Antisense Activity

In certain embodiments, oligomeric compounds and oligomeric duplexes are capable of hybridizing to a target nucleic acid, resulting in at least one antisense activity; such oligomeric compounds and oligomeric duplexes are antisense compounds. In certain embodiments, antisense compounds have antisense activity when they reduce or inhibit the amount or activity of a target nucleic acid by 25% or more in the standard cell assay. In certain embodiments, antisense compounds selectively affect one or more target nucleic acid. Such antisense compounds comprise a nucleobase sequence that hybridizes to one or more target nucleic acid, resulting in one or more desired antisense activity and does not hybridize to one or more non-target nucleic acid or does not hybridize to one or more non-target nucleic acid in such a way that results in significant undesired antisense activity.

In certain antisense activities, hybridization of an antisense compound to a target nucleic acid results in recruitment of a protein that cleaves the target nucleic acid. For example, certain antisense compounds result in RNase H mediated cleavage of the target nucleic acid. RNase H is a cellular endonuclease that cleaves the RNA strand of an RNA:DNA duplex. The DNA in such an RNA:DNA duplex need not be unmodified DNA. In certain embodiments, described herein are antisense compounds that are sufficiently “DNA-like” to elicit RNase H activity. In certain embodiments, one or more non-DNA-like nucleoside in the gap of a gapmer is tolerated.

In certain antisense activities, an antisense compound or a portion of an antisense compound is loaded into an RNA-induced silencing complex (RISC), ultimately resulting in cleavage of the target nucleic acid. For example, certain antisense compounds result in cleavage of the target nucleic acid by Argonaute. Antisense compounds that are loaded into RISC are RNAi compounds. RNAi compounds may be double-stranded (siRNA or dsRNAi) or single-stranded (ssRNA).

In certain embodiments, hybridization of an antisense compound to a target nucleic acid does not result in recruitment of a protein that cleaves that target nucleic acid. In certain embodiments, hybridization of the antisense compound to the target nucleic acid results in alteration of splicing of the target nucleic acid. In certain embodiments, hybridization of an oligomeric compound to a target nucleic acid results in exon inclusion. In certain embodiments, hybridization of an oligomeric compound to a target nucleic acid results in exon exclusion. In certain embodiments, hybridization of an oligomeric compound to a target nucleic acid results in a reduced amount or level of RNA that includes an NIE. In certain embodiments, hybridization of an oligomeric compound to a target nucleic acid results in an increase in the amount or activity of a target nucleic acid. In certain embodiments, hybridization of an antisense compound to a target nucleic acid results in inhibition of a binding interaction between the target nucleic acid and a protein or other nucleic acid. In certain embodiments, hybridization of an antisense compound to a target nucleic acid results in alteration of translation of the target nucleic acid.

Antisense activities may be observed directly or indirectly. In certain embodiments, observation or detection of an antisense activity involves observation or detection of a change in an amount of a target nucleic acid or protein encoded by such target nucleic acid, a change in the ratio of splice variants of a nucleic acid or protein and/or a phenotypic change in a cell or animal.

### III. Certain Target Nucleic Acids

In certain embodiments, oligomeric compounds comprise or consist of a modified oligonucleotide comprising a region that is complementary to a target nucleic acid. In certain embodiments, the target nucleic acid is an endogenous RNA molecule. In certain embodiments, the target nucleic acid encodes a protein. In certain such embodiments, the target nucleic acid is selected from: a mature mRNA and a pre-mRNA, including intronic, exonic, and untranslated regions. In certain embodiments, the target RNA is a mature mRNA. In certain embodiments, the target nucleic acid is a pre-mRNA. In certain embodiments, the target region is entirely within an intron. In certain embodiments, the target region spans an intron/exon junction. In certain embodiments, the target region is at least 50% within an intron.

A. SCN1A In certain embodiments, oligomeric compounds comprise or consist of a modified oligonucleotide that is complementary to a target nucleic acid encoding SCN1A, or a portion thereof. In certain embodiments, the SCN1A target nucleic acid has the nucleobase sequence set forth in SEQ ID NO: 1 (the complement of GENBANK Accession No. NC_000002.12 truncated from nucleotides 165982001 to 166152000). In certain embodiments, the SCN1A target nucleic acid has the nucleobase sequence set forth in SEQ ID NO: 2 (GENBANK Accession No. NM_001165963.2).

In certain embodiments, contacting a cell or subject with an oligomeric compound complementary to SEQ ID NO: 1 or SEQ ID NO: 2 modulates splicing of SCN1A RNA in a cell or a subject. In certain embodiments, contacting a cell or a subject with an oligomeric compound complementary to SEQ ID NO: 1 or SEQ ID NO: 2 increases the amount of SCN1A RNA and/or protein. In certain embodiments, contacting a cell or a subject with an oligomeric compound complementary to SEQ ID NO: 1 or SEQ ID NO: 2 reduces the amount of SCN1A RNA including a NIE. In certain embodiments, contacting a cell or a subject with an oligomeric compound complementary to SEQ ID NO: 1 or SEQ ID NO: 2 increases the amount of SCN1A RNA excluding a NIE. In certain embodiments, the NIE is NIE-1. In certain embodiments, the oligomeric compound comprises or consists of a modified oligonucleotide.

In certain embodiments, contacting a cell in a subject with an oligomeric compound complementary to SEQ ID NO: 1 or SEQ ID NO: 2 ameliorates one or more symptom or hallmark of a disease or disorder associated with SCN1A. In certain embodiments, the disease or disorder associated with SCN1A is a DEE. In certain embodiments, the DEE is Dravet Syndrome. In certain embodiments, the symptom is any of seizures that are prolonged in duration (often lasting longer than 10 minutes), frequent seizures (for example, convulsive, myoclonic, absence, focal, obtundation status, and tonic seizures), sudden unexpected death in epilepsy, status epilepticus, behavioral dysfunctions (for example, aggressiveness, agitation, obsessiveness, preservation, hoarding behavior, or sleep disorders), and developmental delays, movement and balance dysfunctions, orthopedic conditions, motor system and cognitive dysfunctions (for example, ataxia, tremors, dysarthria, pyramidal, and extrapyramidal signs), cognitive impairment, delayed language and speech issues, visual motor integration dysfunctions, visual perception dysfunctions, executive dysfunctions, growth and nutrition issues, sleeping difficulties, chronic infections, sensory integration disorders, and dysautonomia

### B. Certain Target Nucleic Acids in Certain Tissues

In certain embodiments, oligomeric compounds comprise or consist of a modified oligonucleotide comprising a portion that is complementary to a target nucleic acid, wherein the target nucleic acid is expressed in a pharmacologically relevant tissue. In certain embodiments, the pharmacologically relevant tissues are the cells and tissues that comprise the central nervous system (CNS). Such tissues include brain tissues, such as, cerebral cortex, hippocampus, brainstem, and thalamus.

### IV. Certain Pharmaceutical Compositions

In certain embodiments, described herein are pharmaceutical compositions comprising one or more oligomeric compounds. In certain embodiments, the one or more oligomeric compounds each consists of a modified oligonucleotide. In certain embodiments, the pharmaceutical composition comprises a pharmaceutically acceptable diluent or carrier. In certain embodiments, a pharmaceutical composition comprises or consists of a sterile saline solution and one or more oligomeric compound. In certain embodiments, the sterile saline is pharmaceutical grade saline. In certain embodiments, a pharmaceutical composition comprises or consists of one or more oligomeric compound and sterile water. In certain embodiments, the sterile water is pharmaceutical grade water. In certain embodiments, a pharmaceutical composition comprises or consists of one or more oligomeric compound and phosphate-buffered saline (PBS). In certain embodiments, the sterile PBS is pharmaceutical grade PBS. In certain embodiments, a pharmaceutical composition comprises or consists of one or more oligomeric compound and artificial cerebrospinal fluid. In certain embodiments, the artificial cerebrospinal fluid is pharmaceutical grade artificial cerebrospinal fluid.

In certain embodiments, a pharmaceutical composition comprises a modified oligonucleotide and PBS. In certain embodiments, a pharmaceutical composition consists of a modified oligonucleotide and PBS. In certain embodiments, a pharmaceutical composition consists essentially of a modified oligonucleotide and PBS. In certain embodiments, the PBS is pharmaceutical grade.

In certain embodiments, a pharmaceutical composition comprises a modified oligonucleotide and artificial cerebrospinal fluid. In certain embodiments, a pharmaceutical composition consists of a modified oligonucleotide and artificial cerebrospinal fluid. In certain embodiments, a pharmaceutical composition consists essentially of a modified oligonucleotide and artificial cerebrospinal fluid. In certain embodiments, the artificial cerebrospinal fluid is pharmaceutical grade.

In certain embodiments, pharmaceutical compositions comprise one or more oligomeric compound and one or more excipients. In certain embodiments, excipients are selected from water, salt solutions, alcohol, polyethylene glycols, gelatin, lactose, amylase, magnesium stearate, talc, silicic acid, viscous paraffin, hydroxymethylcellulose and polyvinylpyrrolidone.

In certain embodiments, oligomeric compounds may be admixed with pharmaceutically acceptable active and/or inert substances for the preparation of pharmaceutical compositions or formulations. Compositions and methods for the formulation of pharmaceutical compositions depend on a number of criteria, including, but not limited to, route of administration, extent of disease, or dose to be administered.

In certain embodiments, pharmaceutical compositions comprising an oligomeric compound encompass any pharmaceutically acceptable salts of the oligomeric compound, esters of the oligomeric compound, or salts of such esters. In certain embodiments, pharmaceutical compositions comprising oligomeric compounds comprising one or more oligonucleotide, upon administration to an animal, including a human, are capable of providing (directly or indirectly) the biologically active metabolite or residue thereof. Accordingly, for example, the disclosure is also drawn to pharmaceutically acceptable salts of oligomeric compounds, prodrugs, pharmaceutically acceptable salts of such prodrugs, and other bioequivalents. Suitable pharmaceutically acceptable salts include, but are not limited to, sodium, potassium, calcium, and magnesium salts. In certain embodiments, prodrugs comprise one or more conjugate group attached to a modified oligonucleotide, wherein the conjugate group is cleaved by endogenous nucleases within the body.

Lipid moieties have been used in nucleic acid therapies in a variety of methods. In certain such methods, the nucleic acid, such as an oligomeric compound, is introduced into preformed liposomes or lipoplexes made of mixtures of cationic lipids and neutral lipids. In certain methods, DNA complexes with mono- or poly-cationic lipids are formed without the presence of a neutral lipid. In certain embodiments, a lipid moiety is selected to increase distribution of a pharmaceutical agent to a particular cell or tissue. In certain embodiments, a lipid moiety is selected to increase distribution of a pharmaceutical agent to fat tissue. In certain embodiments, a lipid moiety is selected to increase distribution of a pharmaceutical agent to muscle tissue.

In certain embodiments, pharmaceutical compositions comprise a delivery system. Examples of delivery systems include, but are not limited to, liposomes and emulsions. Certain delivery systems are useful for preparing certain pharmaceutical compositions including those comprising hydrophobic compounds. In certain embodiments, certain organic solvents such as dimethylsulfoxide are used.

In certain embodiments, pharmaceutical compositions comprise one or more tissue-specific delivery molecules designed to deliver the one or more pharmaceutical agents comprising an oligomeric compound provided herein to specific tissues or cell types. For example, in certain embodiments, pharmaceutical compositions include liposomes coated with a tissue-specific antibody.

In certain embodiments, pharmaceutical compositions comprise a co-solvent system. Certain of such co-solvent systems comprise, for example, benzyl alcohol, a nonpolar surfactant, a water-miscible organic polymer, and an aqueous phase. In certain embodiments, such co-solvent systems are used for hydrophobic compounds. A non-limiting example of such a co-solvent system is the VPD co-solvent system, which is a solution of absolute ethanol comprising 3% w/v benzyl alcohol, 8% w/v of the nonpolar surfactant Polysorbate 80™ and 65% w/v polyethylene glycol 300. The proportions of such co-solvent systems may be varied considerably without significantly altering their solubility and toxicity characteristics. Furthermore, the identity of co-solvent components may be varied: for example, other surfactants may be used instead of Polysorbate 80™; the fraction size of polyethylene glycol may be varied; other biocompatible polymers may replace polyethylene glycol, e.g., polyvinyl pyrrolidone; and other sugars or polysaccharides may substitute for dextrose.

In certain embodiments, pharmaceutical compositions are prepared for oral administration. In certain embodiments, pharmaceutical compositions are prepared for buccal administration. In certain embodiments, a pharmaceutical composition is prepared for administration by injection (e.g., intravenous, subcutaneous, intramuscular, intrathecal (IT), intracerebroventricular (ICV), etc.). In certain of such embodiments, a pharmaceutical composition comprises a carrier and is formulated in aqueous solution, such as water or physiologically compatible buffers such as Hanks's solution, Ringer's solution, or physiological saline buffer. In certain embodiments, other ingredients are included (e.g., ingredients that aid in solubility or serve as preservatives). In certain embodiments, injectable suspensions are prepared using appropriate liquid carriers, suspending agents and the like. Certain pharmaceutical compositions for injection are presented in unit dosage form, e.g., in ampoules or in multi-dose containers. Certain pharmaceutical compositions for injection are suspensions, solutions, or emulsions in oily or aqueous vehicles, and may contain formulatory agents such as suspending, stabilizing and/or dispersing agents. Certain solvents suitable for use in pharmaceutical compositions for injection include, but are not limited to, lipophilic solvents and fatty oils, such as sesame oil, synthetic fatty acid esters, such as ethyl oleate or triglycerides, and liposomes.

Under certain conditions, certain compounds disclosed herein act as acids. Although such compounds may be drawn or described in protonated (free acid) form or ionized and in association with a cation (salt) form, aqueous solutions of such compounds exist in equilibrium among such forms. For example, a phosphate linkage of an oligonucleotide in aqueous solution exists in equilibrium among free acid, anion and salt forms. Unless otherwise indicated, compounds described herein are intended to include all such forms. Moreover, certain oligonucleotides have several such linkages, each of which is in equilibrium. Thus, oligonucleotides in solution exist in an ensemble of forms at multiple positions all at equilibrium. The term “oligonucleotide” is intended to include all such forms. Drawn structures necessarily depict a single form. Nevertheless, unless otherwise indicated, such drawings are likewise intended to include corresponding forms. Herein, a structure depicting the free acid of a compound followed by the term “or a salt thereof” expressly includes all such forms that may be fully or partially protonated/de-protonated/in association with a cation. In certain instances, one or more specific cation is identified. The cations include, but are not limited to, sodium, potassium, calcium, and magnesium. In certain embodiments, a structure depicting the free acid of a compound followed by the term “or a pharmaceutically acceptable salt thereof” expressly includes all such forms that may be fully or partially protonated/de-protonated/in association with one or more cations selected from sodium, potassium, calcium, and magnesium.

In certain embodiments, modified oligonucleotides or oligomeric compounds are in aqueous solution with sodium. In certain embodiments, modified oligonucleotides or oligomeric compounds are in aqueous solution with potassium. In certain embodiments, modified oligonucleotides or oligomeric compounds are in PBS. In certain embodiments, modified oligonucleotides or oligomeric compounds are in water. In certain such embodiments, the pH of the solution is adjusted with NaOH and/or HCl to achieve a desired pH.

Herein, certain specific doses are described. A dose may be in the form of a dosage unit. For clarity, a dose (or dosage unit) of a modified oligonucleotide or an oligomeric compound in milligrams indicates the mass of the free acid form of the modified oligonucleotide or oligomeric compound. As described above, in aqueous solution, the free acid is in equilibrium with anionic and salt forms. However, for the purpose of calculating dose, it is assumed that the modified oligonucleotide or oligomeric compound exists as a solvent-free, sodium-acetate free, anhydrous, free acid. For example, where a modified oligonucleotide or an oligomeric compound is in solution comprising sodium (e.g., saline), the modified oligonucleotide or oligomeric compound may be partially or fully de-protonated and in association with Na+ ions. However, the mass of the protons are nevertheless counted toward the weight of the dose, and the mass of the Na+ ions are not counted toward the weight of the dose. Thus, for example, a dose, or dosage unit, of 10 mg of Compound No. 1464713, equals the number of fully protonated molecules that weighs 10 mg. This would be equivalent to 10.51 mg of solvent-free, sodium acetate-free, anhydrous sodiated Compound No. 1464713. When an oligomeric compound comprises a conjugate group, the mass of the conjugate group is included in calculating the dose of such oligomeric compound. If the conjugate group also has an acid, the conjugate group is likewise assumed to be fully protonated for the purpose of calculating dose.

In certain embodiments, where an oligomeric compound is in a solution, such as aCSF, comprising sodium, potassium, calcium, and magnesium, the oligomeric compound may be partially or fully de-protonated and in association with sodium, potassium, calcium, and/or magnesium. However, the mass of the protons is nevertheless counted toward the weight of the dose, and the mass of the sodium, potassium, calcium, and magnesium ions is not counted toward the weight of the dose.

In certain embodiments, when an oligomeric compound comprises a conjugate group, the mass of the conjugate group is included in calculating the dose of such oligomeric compound. If the conjugate group also has an acid, the conjugate group is likewise assumed to be fully protonated for the purpose of calculating dose.

### Nonlimiting Disclosure and Incorporation by Reference

Each of the literature and patent publications listed herein is incorporated by reference in its entirety.

While certain compounds, compositions and methods described herein have been described with specificity in accordance with certain embodiments, the following examples serve only to illustrate the compounds described herein and are not intended to limit the same. Each of the references, GenBank accession numbers, ENSEMBL identifiers, and the like recited in the present application is incorporated herein by reference in its entirety.

Although the sequence listing accompanying this filing identifies each sequence as either “RNA” or “DNA” as required, in reality, those sequences may be modified with any combination of chemical modifications. One of skill in the art will readily appreciate that such designation as “RNA” or “DNA” to describe modified oligonucleotides is, in certain instances, arbitrary. For example, an oligonucleotide comprising a nucleoside comprising a 2′-OH sugar moiety and a thymine base could be described as a DNA having a modified sugar (2′-OH in place of one 2′-H of DNA) or as an RNA having a modified base (thymine (methylated uracil) in place of a uracil of RNA). Accordingly, nucleic acid sequences provided herein, including, but not limited to those in the sequence listing, are intended to encompass nucleic acids containing any combination of natural or modified RNA and/or DNA, unless otherwise stated, including, but not limited to such nucleic acids having modified nucleobases. By way of further example and without limitation, an oligomeric compound having the nucleobase sequence “ATCGATCG” encompasses any oligomeric compounds having such nucleobase sequence, whether modified or unmodified, including, but not limited to, such compounds comprising RNA bases, such as those having sequence “AUCGAUCG” and those having some DNA bases and some RNA bases such as “AUCGATCG” and oligomeric compounds having other modified nucleobases, such as “AT^{m}CGAUCG,” wherein ^{m}C indicates a cytosine base comprising a methyl group at the 5-position.

Certain compounds described herein (e.g., modified oligonucleotides) have one or more asymmetric center and thus give rise to enantiomers, diastereomers, and other stereoisomeric configurations that may be defined, in terms of absolute stereochemistry, as (R) or (S), as α or β such as for sugar anomers, or as (D) or (L), such as for amino acids, etc. Compounds provided herein that are drawn or described as having certain stereoisomeric configurations include only the indicated compounds. Compounds provided herein that are drawn or described with undefined stereochemistry include all such possible isomers, including their stereorandom and optically pure forms, unless specified otherwise. Likewise, tautomeric forms of the compounds herein are also included unless otherwise indicated. Unless otherwise indicated, compounds described herein are intended to include corresponding salt forms.

The compounds described herein include variations in which one or more atoms are replaced with a non-radioactive isotope or radioactive isotope of the indicated element. For example, compounds herein that comprise hydrogen atoms encompass all possible deuterium substitutions for each of the ¹H hydrogen atoms. Isotopic substitutions encompassed by the compounds herein include but are not limited to: ²H or ³H in place of ¹H, ¹³C or ¹⁴C in place of ¹²C, ¹⁵N in place of ¹⁴N, ¹⁷O or ¹⁸O in place of 16O and ³³S, ³⁴S, ³⁵S, or ³⁶S in place of ³²S. In certain embodiments, non-radioactive isotopic substitutions may impart new properties on the oligomeric compound that are beneficial for use as a therapeutic or research tool. In certain embodiments, radioactive isotopic substitutions may make the compound suitable for research or diagnostic purposes such as imaging.

### EXAMPLES

The following examples illustrate certain embodiments of the present disclosure and are not limiting. Moreover, where specific embodiments are provided, the inventors have contemplated generic application of those specific embodiments. For example, disclosure of an oligonucleotide having a particular motif provides reasonable support for additional oligonucleotides having the same or similar motif. And, for example, where a particular high-affinity modification appears at a particular position, other high-affinity modifications at the same position are considered suitable, unless otherwise indicated.

### Example 1: Design of Modified Oligonucleotides Complementary to a Human SCN1A Nucleic Acid

Modified oligonucleotides complementary to a human SCN1A nucleic acid were designed and synthesized as indicated in the table below.

Each modified oligonucleotide listed in the tables below is 100% complementary to the human SCN1A genomic sequence, designated herein as SEQ ID NO: 1 (the complement of GENBANK Accession No. NC_000002.12 truncated from nucleotides 165982001 to 166152000), and to the mouse SCN1A genomic sequence, designated herein as SEQ ID NO: 3 (the complement of GENBANK Accession No. NC_000068.7 truncated from nucleotides 66268001 to 66/444,000). “Start site” indicates the 5′-most nucleoside to which the modified oligonucleotide is complementary in the target nucleic acid sequence. “Stop site” indicates the 3′-most nucleoside to which the modified oligonucleotide is complementary in the target nucleic acid sequence.

The modified oligonucleotides in the table below are 18 nucleosides in length. The sugar motif for the modified oligonucleotides in the table below are (from 5′ to 3′): nnnnnnnnnnnnnnnnnn, wherein each “n” represents a 2′-NMA sugar moiety. The internucleoside linkage motif for each modified oligonucleotide is provided in the Internucleoside Linkage Motif (5′ to 3′)″ column in the table below, wherein each “s” represents a phosphorothioate internucleoside linkage and each “o” represents a phosphodiester internucleoside linkage. Each cytosine residue is a 5-methylcytosine.

TABLE 2
2′-NMA modified oligonucleotides with mixed PS/PO internucleoside linkages

| | SEQ ID | SEQ ID | SEQ ID | SEQ ID | | | |
|---|---|---|---|---|---|---|---|
| | NO: 1 | NO: 1 | NO: 3 | NO: 3 | | Internucleoside | SEQ |
| Compound | Start | Stop | Start | Stop | Nucleobase Sequence | Linkage Motif | ID |
| Number | Site | Site | Site | Site | (5′ to 3′) | (5′ to 3′) | NO. |
| 1464713 | 144708 | 144725 | 150106 | 150123 | AGTTGGAGCAAGATTATC | SOOSSSSSSSSSSSSSS | 13 |
| 1464714 | 144708 | 144725 | 150106 | 150123 | AGTTGGAGCAAGATTATC | SOSOSSSSSSSSSSSSS | 13 |
| 1464717 | 144708 | 144725 | 150106 | 150123 | AGTTGGAGCAAGATTATC | SOSSOSSSSSSSSSSSS | 13 |
| 1464718 | 144708 | 144725 | 150106 | 150123 | AGTTGGAGCAAGATTATC | SOSSSOSSSSSSSSSSS | 13 |
| 1464719 | 144708 | 144725 | 150106 | 150123 | AGTTGGAGCAAGATTATC | SOSSSSSOSSSSSSSSS | 13 |
| 1464720 | 144708 | 144725 | 150106 | 150123 | AGTTGGAGCAAGATTATC | SSSOOSSSSSSSSSSSS | 13 |
| 1464721 | 144708 | 144725 | 150106 | 150123 | AGTTGGAGCAAGATTATC | SSSSSSSOOSSSSSSSS | 13 |
| 1464722 | 144708 | 144725 | 150106 | 150123 | AGTTGGAGCAAGATTATC | SSSSSSSSSOOSSSSSS | 13 |
| 1464723 | 144708 | 144725 | 150106 | 150123 | AGTTGGAGCAAGATTATC | SSSSSSSSSSSOOSSSS | 13 |
| 1594953 | 144708 | 144725 | 150106 | 150123 | AGTTGGAGCAAGATTATC | SOSSSSSSSOSSSSSSS | 13 |
| 1594954 | 144708 | 144725 | 150106 | 150123 | AGTTGGAGCAAGATTATC | SOSSSSSSSSSOSSSSS | 13 |
| 1594955 | 144708 | 144725 | 150106 | 150123 | AGTTGGAGCAAGATTATC | SOSSSSSSSSSSSSOSS | 13 |
| 1594956 | 144708 | 144725 | 150106 | 150123 | AGTTGGAGCAAGATTATC | SSSSSOOSSSSSSSSSS | 13 |
| 1594960 | 144708 | 144725 | 150106 | 150123 | AGTTGGAGCAAGATTATC | SSSSSSSSSSSSSOOSS | 13 |
| 1594962 | 144708 | 144725 | 150106 | 150123 | AGTTGGAGCAAGATTATC | SSSSSSOSSSSSSSOSS | 13 |
| 1594963 | 144708 | 144725 | 150106 | 150123 | AGTTGGAGCAAGATTATC | SSSSSSSSOSSSSSOSS | 13 |
| 1594964 | 144708 | 144725 | 150106 | 150123 | AGTTGGAGCAAGATTATC | SSSSSSSSSSOSSSOSS | 13 |
| 1594965 | 144708 | 144725 | 150106 | 150123 | AGTTGGAGCAAGATTATC | SSSSSSSSSSSSOSOSS | 13 |
| 1594966 | 144708 | 144725 | 150106 | 150123 | AGTTGGAGCAAGATTATC | SSSSSSSSOSSSSOSSS | 13 |
| 1594967 | 144708 | 144725 | 150106 | 150123 | AGTTGGAGCAAGATTATC | SSSSSSSSSSSOSOSSS | 13 |

The modified oligonucleotide in the table below is 19 nucleosides in length. The sugar motif for the modified oligonucleotide is (from 5′ to 3′): nnnnnnnnnnnnnnnnnnn, wherein each “n” represents a 2′-NMA sugar moiety. The internucleoside linkage motif for the modified oligonucleotide is (from 5′ to 3′): osssssssssssssssss, wherein each “s” represents a phosphorothioate internucleoside linkage and each “o” represents a phosphodiester internucleoside linkage. Each cytosine residue is a 5-methylcytosine.

TABLE 3
2′-NMA modified oligonucleotide with mixed PS/PO internucleoside linkages

| | SEQ | SEQ | SEQ | SEQ | | |
|---|---|---|---|---|---|---|
| | ID | ID | ID | ID | | |
| | NO: 1 | NO: 1 | NO: 3 | NO: 3 | | SEQ |
| Compound | Start | Stop | Start | Stop | Nucleobase Sequence | ID |
| Number | Site | Site | Site | Site | (5′ to 3′) | NO. |
| 1594968 | 144708 | 144726 | 150106 | 150124 | AAGTTGGAGCAAGATTATC | 14 |

The modified oligonucleotide in the table below is 20 nucleosides in length. The sugar motif for the modified oligonucleotide is (from 5′ to 3′): nnnnnnnnnnnnnnnnnnnn, wherein each “n” represents a 2′-NMA sugar moiety. The internucleoside linkage motif for the modified oligonucleotide is (from 5′ to 3′): ossssssssssssssssso, wherein each “s” represents a phosphorothioate internucleoside linkage and each “o” represents a phosphodiester internucleoside linkage. Each cytosine residue is a 5-methylcytosine.

TABLE 4
2′-NMA modified oligonucleotide with mixed
PS/PO internucleoside linkages

| | SEQ | SEQ | SEQ | SEQ | | |
|---|---|---|---|---|---|---|
| | ID | ID | ID | ID | | |
| | NO: 1 | NO: 1 | NO: 3 | NO: 3 | Nucleobase | SEQ |
| Compound | Start | Stop | Start | Stop | Sequence | ID |
| No. | Site | Site | Site | Site | (5′ to 3′) | No. |
| 1594969 | 144707 | 144726 | 150105 | 150124 | AAGTTGGAGC | 15 |
| | | | | | AAGATTATCC | |

### Example 2: Effect of Modified Oligonucleotides Targeting SCN1A in Wildtype Mice

Wildtype C57BL/6 female mice were divided into groups of 3 mice each. Each mouse received a single ICV bolus of 50 μg of modified oligonucleotide. A group of 4 mice received PBS as a negative control. Compound No. 1429226 is a modified oligonucleotide having a nucleobase sequence of (from 5′ to 3′) AGTTGGAGCAAGATTATC (SEQ ID NO: 13), wherein each nucleoside comprises a 2′-NMA sugar moiety, each internucleoside linkage is a phosphorothioate internucleoside linkage, and each cytosine is a 5-methylcytosine. Comparator compound 1367010 has the nucleobase sequence, sugar motif, and internucleoside linkage motif of Compound Ex 20X+1, previously described in WO 2019/040923 (incorporated herein by reference). Comparator compound 1367010 has a nucleobase sequence of (from 5′ to 3′) AGTTGGAGCAAGATTATC (SEQ ID NO: 13), wherein each nucleoside comprises a 2′-MOE sugar moiety, and each internucleoside linkage is a phosphorothioate internucleoside linkage. Each cytosine in Comparator compound 1367010 is a 5-methylcytosine. SEQ ID NO: 13 is 100% complementary to SEQ ID NO: 1, from Start Site 144708 to Stop Site 144725, and is 100% complementary to SEQ ID NO: 3 from Start Site 150106 to Stop Site 150123. “Start site” indicates the 5′-most nucleoside to which the modified oligonucleotide is complementary in the target nucleic acid sequence. “Stop site” indicates the 3′-most nucleoside to which the modified oligonucleotide is complementary in the target nucleic acid sequence.

Two weeks post treatment, mice were sacrificed and RNA was extracted from cortical brain tissue for real-time qPCR analysis of SCN1A RNA using mouse primer probe set RTS48951 (forward sequence CCCTAAGAGCCTTATCACGATTT, designated herein as SEQ ID NO: 4; reverse sequence GGCAAACCAGAAGCACATTC, designated herein as SEQ ID NO: 5; probe sequence AGGGTGGTTGTGAATGCCCTGTTA, designated herein as SEQ ID NO: 6) to measure the amount of SCN1A RNA that excludes the mouse form of NIE-1 (NIE-1⁻), and mouse primer probe set RTS48949 (forward sequence AGCCCTTTATTATGGGTGGTT, designated herein as SEQ ID NO: 7; reverse sequence CCAGAATATAAGGCAAACCAGAAG, designated herein as SEQ ID NO: 8; probe sequence TGGATGGAATTGCTCCTAACAGGGC, designated herein as SEQ ID NO: 9) to measure the amount of SCN1A transcript that includes the mouse form of NIE-1 (NIE-1⁺). SCN1A RNA is presented as the percent of SCN1A RNA relative to the average of the amount in PBS treated animals (% control), normalized to mouse GAPDH. Mouse GAPDH was amplified using primer probe set mGapdh_LTS00102 (forward sequence GGCAAATTCAACGGCACAGT, designated herein as SEQ ID NO: 10; reverse sequence GGGTCTCGCTCCTGGAAGAT, designated herein as SEQ ID NO: 11; probe sequence AAGGCCGAGAATGGGAAGCTTGTCATC, designated herein as SEQ ID NO: 12).

TABLE 5
Effect of modified oligonucleotides on the amount of
mouse SCN1A excluding NIE-1 (NIE-1⁻), and the amount of
mouse SCN1A including NIE-1 (NIE-1⁺), single dose

Cortex

| Compound No. | RTS48949 NIE-1⁺ | RTS48951 NIE-1⁻ |
|---|---|---|
| PBS | 100 | 100 |
| 1367010 | 58 | 136 |
| 1429226 | 25 | 153 |
| 1464713 | 27 | 154 |
| 1464714 | 20 | 158 |
| 1464717 | 38 | 147 |
| 1464718 | 47 | 134 |
| 1464719 | 36 | 130 |
| 1464720 | 49 | 138 |
| 1464721 | 38 | 136 |
| 1464722 | 34 | 135 |
| 1464723 | 27 | 139 |
| 1594953 | 42 | 150 |
| 1594954 | 35 | 150 |
| 1594955 | 52 | 125 |
| 1594956 | 42 | 124 |
| 1594960 | 49 | 120 |
| 1594962 | 22 | 141 |
| 1594963 | 38 | 130 |
| 1594964 | 55 | 124 |
| 1594965 | 24 | 144 |
| 1594966 | 45 | 132 |
| 1594967 | 58 | 130 |
| 1594968 | 46 | 130 |
| 1594969 | 34 | 118 |

### Example 3: Tolerability of Modified Oligonucleotides Complementary to SCN1A in Wild-Type Mice

Modified oligonucleotides described above were tested in wild-type mice to assess the tolerability of the oligonucleotides.

Wild-type female C57/Bl6 mice each received a single ICV dose of 700 μg of modified oligonucleotide. Each treatment group consisted of 3-4 mice. A group of 4 mice received PBS as a negative control. At 3 hours post-injection, mice were evaluated according to seven different criteria. The criteria are: (1) the mouse was bright, alert, and responsive; (2) the mouse was standing or hunched without stimuli; (3) the mouse showed any movement without stimuli; (4) the mouse demonstrated forward movement after it was lifted; (5) the mouse demonstrated any movement after it was lifted; (6) the mouse responded to tail pinching; (7) regular breathing. For each of the 7 criteria, a mouse was given a subscore of 0 if it met the criteria and 1 if it did not (the functional observational battery score or FOB). After all 7 criteria were evaluated, the scores were summed for each mouse and averaged within each treatment group.

TABLE 6
Tolerability scores in wild-type mice

| Compound No. | FOB 3 hour |
|---|---|
| PBS | 0.00 |
| 1464713 | 1.00 |
| 1464714 | 1.00 |
| 1464717 | 0.67 |
| 1464718 | 1.00 |
| 1464719 | 1.33 |
| 1464720 | 1.00 |
| 1464721 | 1.67 |
| 1464722 | 1.67 |
| 1464723 | 1.67 |
| 1594953 | 1.00 |
| 1594954 | 1.33 |
| 1594955 | 2.33 |
| 1594956 | 2.33 |
| 1594960 | 2.67 |
| 1594962 | 2.00 |
| 1594963 | 2.00 |
| 1594964 | 2.33 |
| 1594965 | 2.67 |
| 1594966 | 2.33 |
| 1594967 | 3.33 |
| 1594968 | 2.33 |
| 1594969 | 1.00 |

TABLE 7
Tolerability scores in wild-type mice

| Compound No. | FOB 3 hour |
|---|---|
| PBS | 0 |
| 1367010 | 7 |

TABLE 8
Tolerability scores in wild-type mice

| Compound No. | FOB 3 hour |
|---|---|
| PBS | 0 |
| 1429226 | 4 |

### Example 4: Tolerability of Modified Oligonucleotides Complementary to Human SCN1A in Rats, 3-Hour Study

Modified oligonucleotides described above were tested in rats to assess the tolerability of the oligonucleotides. Sprague Dawley rats each received a single intrathecal (IT) dose of 3 mg of oligonucleotide listed in the table below. Each treatment group consisted of 3-4 rats. A group of 4 rats received PBS as a negative control. At 3 hours post-injection, movement in 7 different parts of the body were evaluated for each rat. The 7 body parts are: (1) the rat's tail; (2) the rat's posterior posture; (3) the rat's hind limbs; (4) the rat's hind paws; (5) the rat's forepaws; (6) the rat's anterior posture; (7) the rat's head. For each of the 7 different body parts, each rat was given a sub-score of 0 if the body part was moving or 1 if the body part was paralyzed (the functional observational battery score or FOB). After each of the 7 body parts were evaluated, the sub-scores were summed for each rat and then averaged for each group. For example, if a rat's tail, head, and all other evaluated body parts were moving 3 hours after the 3 mg IT dose, it would get a summed score of 0. If another rat was not moving its tail 3 hours after the 3 mg IT dose but all other evaluated body parts were moving, it would receive a score of 1. Results are presented as the average score for each treatment group.

TABLE 9
Tolerability scores in rats

| Compound No. | FOB 3 hour |
|---|---|
| PBS | 0.00 |
| 1464713 | 2.00 |
| 1464714 | 0.33 |
| 1464717 | 1.00 |
| 1464718 | 3.00 |
| 1464719 | 3.00 |
| 1464720 | 3.33 |
| 1464721 | 3.00 |
| 1464722 | 2.67 |
| 1464723 | 2.00 |
| 1594953 | 1.67 |
| 1594954 | 3.33 |
| 1594955 | 3.33 |
| 1594956 | 2.67 |
| 1594960 | 4.00 |
| 1594962 | 3.00 |
| 1594963 | 3.67 |
| 1594964 | 2.00 |
| 1594965 | 3.33 |
| 1594966 | 3.67 |
| 1594967 | 2.33 |
| 1594968 | 2.67 |
| 1594969 | 2.67 |

TABLE 10
Tolerability scores in rats

| Compound No. | FOB 3 hour |
|---|---|
| PBS | 0.25 |
| 1367010 | 6 |

TABLE 11
Tolerability scores in rats

| Compound No. | FOB 3 hour |
|---|---|
| PBS | 0 |
| 1429226 | 4 |

### Example 5: Design of Modified Oligonucleotides Complementary to a SCN1A Nucleic Acid

Modified oligonucleotides complementary to a SCN1A nucleic acid were designed and synthesized as indicated in the tables below.

The modified oligonucleotides listed in the table below are 100% complementary to the human SCN1A genomic sequence, designated herein as SEQ TD NO: 1 (described herein above), and to the mouse SCN1A genomic sequence, designated herein as SEQ ID NO: 3 (described herein above). “Start site” indicates the 5′-most nucleoside to which the modified oligonucleotide is complementary in the target nucleic acid sequence. “Stop site” indicates the 3′-most nucleoside to which the modified oligonucleotide is complementary in the target nucleic acid sequence. “N.A.” indicates that the modified oligonucleotide is not 100% complementary to the target nucleic acid sequence.

The modified oligonucleotides in the table below are 18 nucleosides in length. The sugar motif for the modified oligonucleotides in the table below are (from 5′ to 3′): nnnnnnnnnnnnnnnnmn, wherein each “n” ˜ represents a 2′-NMA sugar moiety. The internucleoside linkage motif for the modified oligonucleotides is (from 5′ to 3′): sssssssssssssssss; wherein each “s” represents a phosphorothioate internucleoside linkage. Each cytosine residue is a 5-methylcytosine.

TABLE 12
2′-NMA modified oligonucleotides with uniform phosphorothioate internucleoside linkages

| | SEQ ID | SEQ ID | SEQ ID | SEQ ID | | |
|---|---|---|---|---|---|---|
| | NO: 1 | NO: 1 | NO: 3 | NO: 3 | | |
| Compound | Start | Stop | Start | Stop | | SEQ ID |
| Number | Site | Site | Site | Site | Sequence (5′ to 3′) | NO. |
| 1521407 | 144779 | 144796 | 150177 | 150194 | GGTAGCAAAAGGGGTAAT | 63 |
| 1521408 | 144776 | 144793 | 150174 | 150191 | AGCAAAAGGGGTAATACA | 64 |
| 1521409 | 144775 | 144792 | 150173 | 150190 | GCAAAAGGGGTAATACAG | 65 |
| 1521410 | 144774 | 144791 | 150172 | 150189 | CAAAAGGGGTAATACAGT | 66 |
| 1521411 | 144772 | 144789 | 150170 | 150187 | AAAGGGGTAATACAGTAC | 67 |
| 1521412 | 144771 | 144788 | 150169 | 150186 | AAGGGGTAATACAGTACC | 68 |
| 1521413 | 144766 | 144783 | 150164 | 150181 | GTAATACAGTACCCATAA | 69 |
| 1521414 | 144765 | 144782 | 150163 | 150180 | TAATACAGTACCCATAAT | 70 |
| 1521415 | 144764 | 144781 | 150162 | 150179 | AATACAGTACCCATAATA | 71 |
| 1521416 | 144763 | 144780 | 150161 | 150178 | ATACAGTACCCATAATAA | 72 |
| 1521417 | 144762 | 144779 | 150160 | 150177 | TACAGTACCCATAATAAA | 73 |
| 1521418 | 144761 | 144778 | 150159 | 150176 | ACAGTACCCATAATAAAG | 74 |
| 1521419 | 144716 | 144733 | 150114 | 150131 | CCCATCCAAGTTGGAGCA | 75 |
| 1521420 | 144715 | 144732 | 150113 | 150130 | CCATCCAAGTTGGAGCAA | 76 |
| 1521421 | 144714 | 144731 | 150112 | 150129 | CATCCAAGTTGGAGCAAG | 77 |
| 1521422 | 144713 | 144730 | 150111 | 150128 | ATCCAAGTTGGAGCAAGA | 78 |
| 1521423 | 144712 | 144729 | 150110 | 150127 | TCCAAGTTGGAGCAAGAT | 79 |
| 1521424 | 144711 | 144728 | 150109 | 150126 | CCAAGTTGGAGCAAGATT | 80 |
| 1521425 | 144710 | 144727 | 150108 | 150125 | CAAGTTGGAGCAAGATTA | 81 |
| 1521426 | 144709 | 144726 | 150107 | 150124 | AAGTTGGAGCAAGATTAT | 82 |
| 1521428 | 144706 | 144723 | 150104 | 150121 | TTGGAGCAAGATTATCCT | 83 |
| 1521429 | 144705 | 144722 | 150103 | 150120 | TGGAGCAAGATTATCCTA | 84 |
| 1521430 | 144704 | 144721 | 150102 | 150119 | GGAGCAAGATTATCCTAT | 85 |
| 1521431 | 144703 | 144720 | 150101 | 150118 | GAGCAAGATTATCCTATA | 86 |

The modified oligonucleotides in the table below are 25 nucleosides in length. The sugar motif for the modified oligonucleotides in the table below are (from 5′ to 3′): nnnnnnnnnnnnnnnnnnnnnnnu, wherein each “n” represents a 2′-NMA sugar moiety. The internucleoside linkage motif for the modified oligonucleotides is (from 5′ to 3′): ssoosoosoossssssssssssss; wherein each “s” represents a phosphorothioate internucleoside linkage and each “o” represents a phosphodiester internucleoside linkage. Each cytosine residue is a 5-methylcytosine.

TABLE 13
2′-NMA modified oligonucleotides with mixed PS/PO internucleoside linkages

| | SEQ ID | SEQ ID | SEQ ID | SEQ ID | | |
|---|---|---|---|---|---|---|
| | NO: 1 | NO: 1 | NO: 3 | NO: 3 | | SEQ |
| Compound | Start | Stop | Start | Stop | | ID |
| Number | Site | Site | Site | Site | Sequence (5′ to 3′) | NO. |
| 1669084 | 144706 | 144730 | 150104 | 150128 | ATCCAAGTTGGAGCAAGATTATCCT | 19 |
| 1669085 | 144705 | 144729 | 150103 | 150127 | TCCAAGTTGGAGCAAGATTATCCTA | 20 |
| 1669086 | 144704 | 144728 | 150102 | 150126 | CCAAGTTGGAGCAAGATTATCCTAT | 21 |
| 1669087 | 144703 | 144727 | 150101 | 150125 | CAAGTTGGAGCAAGATTATCCTATA | 22 |

The modified oligonucleotides in the table below are 25 nucleosides in length. The sugar motif for the modified oligonucleotides in the table below are (from 5′ to 3′): nnnnnnnnnnnnnnnnnnn wherein each “n” represents a 2′-NMA sugar moiety. The internucleoside linkage motif for the modified oligonucleotides is (from 5′ to 3′): ssoosoosssssssssssssssss; wherein each “s” represents a phosphorothioate internucleoside linkage and each “o” represents a phosphodiester internucleoside linkage. Each cytosine residue is a 5-methylcytosine.

TABLE 14
2′-NMA modified oligonucleotides with mixed
PS/PO internucleoside linkages

| | SEQ ID | SEQ ID | SEQ ID | SEQ ID | | |
|---|---|---|---|---|---|---|
| | NO: 1 | NO: 1 | NO: 3 | NO: 3 | | SEQ |
| Compound | Start | Stop | Start | Stop | | ID |
| Number | Site | Site | Site | Site | Sequence (5′ to 3′) | NO. |
| 1669088 | 144706 | 144730 | 150104 | 150128 | ATCCAAGTTGGAGCAAGATTATCCT | 19 |
| 1669089 | 144705 | 144729 | 150103 | 150127 | TCCAAGTTGGAGCAAGATTATCCTA | 20 |
| 1669090 | 144704 | 144728 | 150102 | 150126 | CCAAGTTGGAGCAAGATTATCCTAT | 21 |
| 1669091 | 144703 | 144727 | 150101 | 150125 | CAAGTTGGAGCAAGATTATCCTATA | 22 |

The modified oligonucleotides in the table below are 25 nucleosides in length. The sugar motif for the modified oligonucleotides in the table below are (from 5′ to 3′): nnnnnnnnnnnnnnnnnnn wherein each “n” represents a 2′-NMA sugar moiety. The internucleoside linkage motif for the modified oligonucleotides is (from 5′ to 3′): ssoossssssssssssssssssss; wherein each “s” represents a phosphorothioate internucleoside linkage and each “o” represents a phosphodiester internucleoside linkage. Each cytosine residue is a 5-methylcytosine.

TABLE 15
2′-NMA modified oligonucleotides
with mixed PS/PO internucleoside linkages

| | SEQ ID | SEQ ID | SEQ ID | SEQ ID | | |
|---|---|---|---|---|---|---|
| | NO: 1 | NO: 1 | NO: 3 | NO: 3 | | SEQ |
| Compound | Start | Stop | Start | Stop | Sequence | ID |
| Number | Site | Site | Site | Site | (5′ to 3′) | NO. |
| 1669093 | 144706 | 144730 | 150104 | 150128 | ATCCAAGTTGGAG | 19 |
| | | | | | CAAGATTATCCT | |
| 1669094 | 144705 | 144729 | 150103 | 150127 | TCCAAGTTGGAGC | 20 |
| | | | | | AAGATTATCCTA | |
| 1669095 | 144704 | 144728 | 150102 | 150126 | CCAAGTTGGAGCA | 21 |
| | | | | | AGATTATCCTAT | |
| 1669096 | 144703 | 144727 | 150101 | 150125 | CAAGTTGGAGCAA | 22 |
| | | | | | GATTATCCTATA | |

The modified oligonucleotides in the table below are 25 nucleosides in length. The sugar motif for the modified oligonucleotides in the table below are (from 5′ to 3′): nnnnnnnnnnnnnnnnnnnn, wherein each “n” represents a 2′-NMA sugar moiety. The internucleoside linkage motif for the modified oligonucleotides is (from 5′ to 3′): ssssssssssssssssssssssss; wherein each “s” represents a phosphorothioate internucleoside linkage. Each cytosine residue is a 5-methylcytosine.

TABLE 16
2′-NMA modified oligonucleotides with uniform phosphorothioate internucleoside linkages

| | SEQ ID | SEQ ID | SEQ ID | SEQ ID | | |
|---|---|---|---|---|---|---|
| | NO: 1 | NO: 1 | NO: 3 | NO: 3 | | |
| Compound | Start | Stop | Start | Stop | | SEQ ID |
| Number | Site | Site | Site | Site | Sequence (5′ to 3′) | NO. |
| 1669098 | 144706 | 144730 | 150104 | 150128 | ATCCAAGTTGGAGCAAGATTATCCT | 19 |
| 1669099 | 144705 | 144729 | 150103 | 150127 | TCCAAGTTGGAGCAAGATTATCCTA | 20 |
| 1669100 | 144704 | 144728 | 150102 | 150126 | CCAAGTTGGAGCAAGATTATCCTAT | 21 |
| 1669101 | 144703 | 144727 | 150101 | 150125 | CAAGTTGGAGCAAGATTATCCTATA | 22 |

The modified oligonucleotides in the table below are 23 nucleosides in length. The sugar motif for the modified oligonucleotides in the table below are (from 5′ to 3′): nnnnnnnnnnnnnnnnnnnnnnn, wherein each “n” represents a 2′-NMA sugar moiety. The internucleoside linkage motifs for the modified oligonucleotides are presented in the column labeled “Internucleoside Linkages (5′ to 3′)” in the table below, wherein each “s” represents a phosphorothioate internucleoside linkage and each “o” represents a phosphodiester internucleoside linkage. Each cytosine residue is a 5-methylcytosine.

TABLE 17
2′-NMA modified oligonucleotides with mixed
PS/PO internucleoside linkages

| | | | | | | Inter- | |
|---|---|---|---|---|---|---|---|
| | | | | | | nucle- | |
| | SEQ | SEQ | SEQ | SEQ | | oside | |
| | ID | ID | ID | ID | Se- | Link- | |
| Com- | NO: 1 | NO: 1 | NO: 3 | NO: 3 | quence | ages | SEQ |
| pound | Start | Stop | Start | Stop | (5′ | (5′ | ID |
| Number | Site | Site | Site | Site | to 3′) | to 3′) | NO. |
| 1669097 | 144774 | 144796 | 150172 | 150194 | GGTAGC | SSOOSS | 18 |
| | | | | | AAAAGG | SSSSSS | |
| | | | | | GGTAAT | SSSSSS | |
| | | | | | ACAGT | SSSS | |
| 1669102 | 144774 | 144796 | 150172 | 150194 | GGTAGC | SSSSSS | 18 |
| | | | | | AAAAGG | SSSSSS | |
| | | | | | GGTAAT | SSSSSS | |
| | | | | | ACAGT | SSSS | |

“Start site” indicates the 5′-most nucleoside to which the modified oligonucleotide is complementary in the target nucleic acid sequence. “Stop site” indicates the 3′-most nucleoside to which the modified oligonucleotide is complementary in the target nucleic acid sequence. The modified oligonucleotides listed in the table below are 100% complementary to the mouse SCN1A sequence designated herein as SEQ ID NO: 3 (described herein above); the sequences are complementary to the human SCN1A sequence of SEQ ID NO: 1 (described herein above) with a single mismatch located at the position indicated in the column labeled “Position of mismatch on Compound (5′ to 3′)”. The non-complementary nucleobases are marked in the Nucleobase Sequence column in underlined, bold, italicized font. Additionally, the modified oligonucleotides listed in the table below are 100% complementary to the mouse SCN1A genomic sequence, designated herein as SEQ ID NO: 3 (described herein above).

The modified oligonucleotides in the table below are 18 nucleosides in length. The sugar motif for the modified oligonucleotides in the table below are (from 5′ to 3′): nnnnnnnnnnnnnnnnnn, wherein each “n” represents a 2′-NMA sugar moiety. The internucleoside linkage motif for the modified oligonucleotides is (from 5′ to 3′): sssssssssssssssss; wherein each “s” represents a phosphorothioate internucleoside linkage. Each cytosine residue is a 5-methylcytosine.

TABLE 18
2′-NMA modified oligonucleotides with uniform
phosphorothioate internucleoside linkages

| | | | Posi- | | | | |
|---|---|---|---|---|---|---|---|
| | | | tion | | | | |
| | | | of | | | | |
| | | | mis- | | | | |
| | | | match | | | | |
| | | | of | | | | |
| | | | mod- | | | | |
| | | | ified | | | | |
| | | | oli- | | | | |
| | | | gonu- | | | | |
| | | | cleo- | | | | |
| | | | tide | | | | |
| | | | on | | | | |
| | | | SEQ | | | Nu- | |
| | | | ID | | | cleo- | |
| | SEQ | SEQ | NO: | SEQ | SEQ | base | |
| | ID | ID | 1 | ID | ID | Se- | |
| Com- | NO: 1 | NO: 1 | (5′ | NO: 3 | NO: 3 | quence | SEQ |
| pound | Start | Stop | to | Start | Stop | (5′ to | ID |
| Number | Site | Site | 3′) | Site | Site | 3′) | NO. |
| 1521432 | 144702 | 144718 | 18 | 150100 | 150117 | AGCAAG | 87 |
| | | | | | | ATTATC | |
| | | | | | | CTATAT | |
| 1521433 | 144701 | 144718 | 17 | 150099 | 150116 | GCAAGA | 88 |
| | | | | | | TTATCC | |
| | | | | | | TATATA | |
| 1521434 | 144699 | 144716 | 15 | 150097 | 150114 | AAGATT | 89 |
| | | | | | | ATCCTA | |
| | | | | | | TATAAA | |

### Example 6: Effect of Modified Oligonucleotides Targeting SCN1A in Wildtype Mice

Wildtype C57BL/6 mice were divided into groups of 3 mice each. Each mouse received a single ICV bolus of 50 μg of modified oligonucleotide. A group of 4 mice received PBS as a negative control.

Two weeks post treatment, mice were sacrificed, and RNA was extracted from cortical brain tissue for real-time qPCR analysis of SCN1A RNA using mouse primer probe set RTS48951 (described herein above) to measure the amount of SCN1A RNA that excludes the mouse form of NIE-1 (NIE-1⁻), and mouse primer probe set RTS48949 (described herein above) to measure the amount of SCN1A transcript that includes the mouse form of NIE-1 (NIE-1⁺). SCN1A RNA is presented as the percent of SCN1A RNA relative to the average of the amount in PBS treated animals (% control), normalized to mouse GAPDH. Mouse GAPDH was amplified using primer probe set mGapdh_LTS00102 (described herein above). Values marked with a “†” result from oligonucleotides that are complementary to the amplicon region of the primer probe set. Additional assays may be used to measure the potency and efficacy of the modified oligonucleotides complementary to the amplicon region.

Comparator compound 1367010 is described herein above.

TABLE 19
Effect of modified oligonucleotides on the amount of
mouse SCN1A excluding NIE-1 (NIE-1⁻), and the amount of
mouse SCN1A including NIE-1 (NIE-1⁺), single dose

SCN1A RNA (% control)

| Compound | RTS48949 | RTS48951 |
|---|---|---|
| No. | NIE-1⁺ | NIE-1⁻ |
| PBS | 100 | 100 |
| 1367010 | 45 | 120 |
| 1429226 | 19 | 142 |
| 1521407 | 57 | 129 |
| 1521408 | 37 | 131 |
| 1521409 | 46 | 126 |
| 1521410 | 43 | 135 |
| 1521411 | 64 | 125 |
| 1521412 | 60† | 123 |
| 1521413 | 52† | 117 |
| 1521414 | 65† | 109 |
| 1521415 | 59† | 143 |
| 1521416 | 56† | 113 |
| 1521417 | 56† | 106 |
| 1521418 | 55† | 110 |
| 1521419 | 60 | 125 |
| 1521420 | 44 | 114 |
| 1521421 | 40 | 134 |
| 1521422 | 57 | 116 |
| 1521423 | 53 | 132 |
| 1521424 | 41 | 120 |
| 1521425 | 26 | 128 |
| 1521426 | 34 | 119 |
| 1521428 | 43 | 119 |
| 1521429 | 44 | 129 |
| 1521430 | 50 | 117 |
| 1521431 | 30 | 111 |
| 1521432 | 30 | 137 |
| 1521433 | 48 | 118 |
| 1521434 | 90 | 98 |

TABLE 20
Effect of modified oligonucleotides on the amount of
mouse SCN1A excluding NIE-1 (NIE-1⁻), and the amount of
mouse SCN1A including NIE-1 (NIE-1⁺), single dose

Cortex

| Compound | RTS48949 | RTS48951 |
|---|---|---|
| No. | NIE-1⁺ | NIE-1⁻ |
| PBS | 100 | 100 |
| 1367010 | 52 | 121 |
| 1429226 | 29 | 154 |
| 1669084 | 86 | 112 |
| 1669085 | 74 | 108 |
| 1669086 | 69 | 120 |
| 1669087 | 78 | 113 |
| 1669088 | 45 | 139 |
| 1669089 | 60 | 123 |
| 1669090 | 62 | 113 |
| 1669091 | 57 | 126 |
| 1669093 | 45 | 123 |

TABLE 21
Effect of modified oligonucleotides on the amount of
mouse SCN1A excluding NIE-1 (NIE-1⁻), and the amount of
mouse SCN1A including NIE-1 (NIE-1⁺), single dose

Cortex

| Compound | RTS48949 | RTS48951 |
|---|---|---|
| No. | NIE-1⁺ | NIE-1⁻ |
| PBS | 100 | 100 |
| 1367010 | 46 | 126 |
| 1429226 | 28 | 116 |
| 1669094 | 60 | 109 |
| 1669095 | 58 | 108 |
| 1669096 | 58 | 135 |
| 1669097 | 69 | 114 |
| 1669098 | 58 | 121 |
| 1669099 | 60 | 108 |
| 1669100 | 61 | 121 |
| 1669101 | 56 | 106 |
| 1669102 | 48 | 109 |

### Example 7: Potency of Modified Oligonucleotides Targeting SCN1A in Wildtype Mice

Wildtype C57BL/6 female mice were divided into groups of 4 mice each. Each mouse received a single ICV bolus of modified oligonucleotide at various doses defined in the tables below. A group of 4 mice received PBS as a negative control.

Two weeks post treatment, mice were sacrificed, and RNA was extracted from cortical brain tissue for real-time qPCR analysis of SCN1A RNA using mouse primer probe set RTS48951 (described herein above) to measure the amount of SCN1A RNA that excludes the mouse form of NIE-1 (NIE-1⁻) and mouse primer probe set RTS48949 (described herein above) to measure the amount of SCN1A transcript that includes the mouse form of NIE-1 (NIE-1+) SCN1A RNA is presented as the percent of SCN1A RNA relative to the average of the amount in PBS treated animals (% control), normalized to mouse GAPDH. Mouse GAPDH was amplified using primer probe set mGapdh_LTS00102 (described herein above). ED50s were calculated in using GraphPad Prism.

Comparator compound 1367010 is described herein above.

TABLE 22
Effect of modified oligonucleotides on the amount of mouse
SCN1A excluding NIE-1 (NIE-1⁻) and the amount of mouse SCN1A
RNA including (NIE-1⁺) in wildtype mice, multiple doses

| | | Cortex | | |
|---|---|---|---|---|
| | Dose | RTS48951 NIE-1⁻ | RTS48949 NIE-1⁺ | |
| Compound No. | (μg) | % Control | % Control | ED50 (μg) |
| PBS | — | 100 | 100 | — |
| 1429226 | 1 | 91 | 83 | 28 |
| | 3 | 103 | 102 | |
| | 10 | 94 | 74 | |
| | 30 | 115 | 62 | |
| | 100 | 168 | 17 | |
| | 300 | 134 | 8 | |
| 1464713 | 1 | 90 | 101 | 63 |
| | 3 | 95 | 107 | |
| | 10 | 90 | 94 | |
| | 30 | 101 | 72 | |
| | 100 | 107 | 47 | |
| | 300 | 129 | 12 | |
| 1464714 | 1 | 92 | 109 | 31 |
| | 3 | 103 | 105 | |
| | 10 | 101 | 83 | |
| | 30 | 116 | 59 | |
| | 100 | 134 | 19 | |
| | 300 | 141 | 12 | |
| 1594962 | 1 | 99 | 108 | 29 |
| | 3 | 97 | 98 | |
| | 10 | 100 | 79 | |
| | 30 | 101 | 59 | |
| | 100 | 114 | 18 | |
| | 300 | 150 | 6 | |
| 1594965 | 1 | 96 | 111 | 31 |
| | 3 | 89 | 106 | |
| | 10 | 105 | 91 | |
| | 30 | 122 | 55 | |
| | 100 | 126 | 22 | |
| | 300 | 138 | 4 | |

TABLE 23
Effect of modified oligonucleotides on the amount of mouse
SCN1A excluding NIE-1 (NIE-1⁻) and the amount of mouse SCN1A
RNA including (NIE-1⁺) in wildtype mice, multiple doses

| | | Cortex | | |
|---|---|---|---|---|
| | | RTS48951 NIE-1⁻ | RTS48949 NIE-1⁺ | |
| Compound No. | Dose (μg) | % control | % control | ED50 (μg) |
| PBS | — | 100 | 100 | — |
| 1464717 | 1 | 110 | 82 | 26 |
| | 3 | 95 | 77 | |
| | 10 | 80 | 77 | |
| | 30 | 93 | 54 | |
| | 100 | 110 | 44 | |
| | 300 | 118 | 10 | |
| 1464723 | 1 | 66 | 83 | 33 |
| | 3 | 81 | 97 | |
| | 10 | 91 | 92 | |
| | 30 | 73 | 55 | |
| | 100 | 107 | 27 | |
| | 300 | 119 | 9 | |
| 1594953 | 1 | 63 | 83 | 35 |
| | 3 | 78 | 82 | |
| | 10 | 88 | 89 | |
| | 30 | 95 | 59 | |
| | 100 | 112 | 37 | |
| | 300 | 117 | 5 | |
| 1594954 | 1 | 89 | 71 | 14 |
| | 3 | 85 | 84 | |
| | 10 | 85 | 68 | |
| | 30 | 100 | 46 | |
| | 100 | 104 | 20 | |
| | 300 | 96 | 4 | |

### Example 8: Tolerability of Modified Oligonucleotides Complementary to SCN1A in Wild-Type Mice

Modified oligonucleotides described above were tested in wild-type mice to assess the tolerability of the oligonucleotides.

Wild-type female C57BL/6 mice each received a single ICV dose of 700 μg of modified oligonucleotide as indicated in the tables below. Each treatment group consisted of 3-4 mice. A group of 4 mice received PBS as a negative control. At 3 hours post-injection, mice were evaluated according to seven different criteria. The criteria are (1) the mouse was bright, alert, and responsive; (2) the mouse was standing or hunched without stimuli; (3) the mouse showed any movement without stimuli; (4) the mouse demonstrated forward movement after it was lifted; (5) the mouse demonstrated any movement after it was lifted; (6) the mouse responded to tail pinching; (7) regular breathing. For each of the 7 criteria, a mouse was given a subscore of 0 if it met the criteria and 1 if it did not (the functional observational battery score or FOB). After all 7 criteria were evaluated, the scores were summed for each mouse and averaged within each treatment group.

TABLE 24
Tolerability scores in wild-type mice at a dose of 700 μg

| Compound No. | FOB 3 hour |
|---|---|
| PBS | 0.00 |
| 1367010 | 7 |

TABLE 25
Tolerability scores in wild-type mice at a dose of 700 μg

| Compound No. | FOB 3 hour |
|---|---|
| PBS | 0.00 |
| 1429226 | 4.00 |

TABLE 26
Tolerability scores in wild-type mice at a dose of 700 μg

| Compound No. | FOB 3 hour |
|---|---|
| PBS | 0.00 |
| 1464713 | 1.00 |
| 1464714 | 1.00 |
| 1464717 | 0.67 |
| 1464718 | 1.00 |
| 1464719 | 1.33 |
| 1464720 | 1.00 |
| 1464721 | 1.67 |
| 1464722 | 1.67 |
| 1464723 | 1.67 |
| 1594953 | 1.00 |
| 1594954 | 1.33 |
| 1594955 | 2.33 |
| 1594956 | 2.33 |
| 1594960 | 2.67 |
| 1594962 | 2.00 |
| 1594963 | 2.00 |
| 1594964 | 2.33 |
| 1594965 | 2.67 |
| 1594966 | 2.33 |
| 1594967 | 3.33 |
| 1594968 | 2.33 |
| 1594969 | 1.00 |

TABLE 27
Tolerability scores in wild-type mice at a dose of 700 μg

| Compound No. | FOB 3 hour |
|---|---|
| PBS | 0.00 |
| 1669084 | 0.00 |
| 1669085 | 0.00 |
| 1669086 | 0.00 |
| 1669091 | 0.00 |
| 1669093 | 0.00 |

### Example 9: Tolerability of Modified Oligonucleotides Complementary to Human SCN1A in Rats, 3-Hour Study

Modified oligonucleotides described above were tested in rats to assess the tolerability of the oligonucleotides. Sprague Dawley rats each received a single intrathecal (IT) dose of 3 mg of oligonucleotide as indicated in the tables below. Each treatment group consisted of 4 rats. A group of 4 rats received PBS as a negative control. At 3 hours post-injection, movement in 7 different parts of the body were evaluated for each rat. The 7 body parts are (1) the rat's tail; (2) the rat's posterior posture; (3) the rat's hind limbs; (4) the rat's hind paws; (5) the rat's forepaws; (6) the rat's anterior posture; (7) the rat's head. For each of the 7 different body parts, each rat was given a sub-score of 0 if the body part was moving or 1 if the body part was paralyzed (the functional observational battery score or FOB). After each of the 7 body parts were evaluated, the sub-scores were summed for each rat and then averaged for each group. For example, if a rat's tail, head, and all other evaluated body parts were moving 3 hours after the 3 mg IT dose, it would get a summed score of 0. If another rat was not moving its tail 3 hours after the 3 mg IT dose but all other evaluated body parts were moving, it would receive a score of 1. Results are presented as the average score for each treatment group.

TABLE 28
Tolerability scores in rats at a dose of 3 mg

| Compound No. | FOB 3 hour |
|---|---|
| PBS | 0.00 |
| 1367010 | 6.00 |

TABLE 29
Tolerability scores in rats at a dose of 3 mg

| Compound No. | FOB 3 hour |
|---|---|
| PBS | 0.00 |
| 1429226 | 4.00 |

TABLE 30
Tolerability scores in rats at a dose of 3 mg

| Compound No. | FOB 3 hour |
|---|---|
| PBS | 0.00 |
| 1464713 | 2.00 |
| 1464714 | 0.33 |
| 1464717 | 1.00 |
| 1464718 | 3.00 |
| 1464719 | 3.00 |
| 1464720 | 3.33 |
| 1464721 | 3.00 |
| 1464722 | 2.67 |
| 1464723 | 2.00 |
| 1594953 | 1.67 |
| 1594954 | 3.33 |
| 1594955 | 3.33 |
| 1594956 | 2.67 |
| 1594960 | 4.00 |
| 1594962 | 3.00 |
| 1594963 | 3.67 |
| 1594964 | 2.00 |
| 1594965 | 3.33 |
| 1594966 | 3.67 |
| 1594967 | 2.33 |
| 1594968 | 2.67 |
| 1594969 | 2.67 |

1. An oligomeric compound comprising a modified oligonucleotide according to the following chemical notation:
(SEQ ID NO: 23)
AₙₛGₙₒTₙₒTₙₛGₙₛGₙₛAₙₛGₙₛ^{m}CₙₛAₙₛAₙₛGₙₛAₙₛTₙₛTₙₛAₙₛTₙₛ^{m}Cₙ;
(SEQ ID NO: 24)
AₙₛGₙₒTₙₛTₙₒGₙₛGₙₛAₙₛGₙₛ^{m}CₙₛAₙₛAₙₛGₙₛAₙₛTₙₛTₙₛAₙₛTₙₛ^{m}Cₙ;
(SEQ ID NO: 25)
AₙₛGₙₒTₙₛTₙₛGₙₒGₙₛAₙₛGₙₛ^{m}CₙₛAₙₛAₙₛGₙₛAₙₛTₙₛTₙₛAₙₛTₙₛ^{m}Cₙ;
(SEQ ID NO: 26)
AₙₛGₙₒTₙₛTₙₛGₙₛGₙₒAₙₛGₙₛ^{m}CₙₛAₙₛAₙₛGₙₛAₙₛTₙₛTₙₛAₙₛTₙₛ^{m}Cₙ;
(SEQ ID NO: 27)
AₙₛGₙₒTₙₛTₙₛGₙₛGₙₛAₙₛGₙₒ^{m}CₙₛAₙₛAₙₛGₙₛAₙₛTₙₛTₙₛAₙₛTₙₛ^{m}Cₙ;
(SEQ ID NO: 28)
AₙₛGₙₛTₙₛTₙₒGₙₒGₙₛAₙₛGₙₛ^{m}CₙₛAₙₛAₙₛGₙₛAₙₛTₙₛTₙₛAₙₛTₙₛ^{m}Cₙ;
(SEQ ID NO: 29)
AₙₛGₙₛTₙₛTₙₛGₙₛGₙₛAₙₛGₙₒ^{m}CₙₒAₙₛAₙₛGₙₛAₙₛTₙₛTₙₛAₙₛTₙₛ^{m}Cₙ;
(SEQ ID NO: 30)
AₙₛGₙₛTₙₛTₙₛGₙₛGₙₛAₙₛGₙₛ^{m}CₙₛAₙₒAₙₒGₙₛAₙₛTₙₛTₙₛAₙₛTₙₛ^{m}Cₙ;
(SEQ ID NO: 31)
AₙₛGₙₛTₙₛTₙₛGₙₛGₙₛAₙₛGₙₛ^{m}CₙₛAₙₛAₙₛGₙₒAₙₒTₙₛTₙₛAₙₛTₙₛ^{m}Cₙ;
(SEQ ID NO: 32)
AₙₛGₙₒTₙₛTₙₛGₙₛGₙₛAₙₛGₙₛ^{m}CₙₛAₙₒAₙₛGₙₛAₙₛTₙₛTₙₛAₙₛTₙₛ^{m}Cₙ;
(SEQ ID NO: 33)
AₙₛGₙₒTₙₛTₙₛGₙₛGₙₛAₙₛGₙₛ^{m}CₙₛAₙₛAₙₛGₙₒAₙₛTₙₛTₙₛAₙₛTₙₛ^{m}Cₙ;
(SEQ ID NO: 34)
AₙₛGₙₒTₙₛTₙₛGₙₛGₙₛAₙₛGₙₛ^{m}CₙₛAₙₛAₙₛGₙₛAₙₛTₙₛTₙₒAₙₛTₙₛ^{m}Cₙ;
(SEQ ID NO: 35)
AₙₛGₙₛTₙₛTₙₛGₙₛGₙₒAₙₒGₙₛ^{m}CₙₛAₙₛAₙₛGₙₛAₙₛTₙₛTₙₛAₙₛTₙₛ^{m}Cₙ;
(SEQ ID NO: 36)
AₙₛGₙₛTₙₛTₙₛGₙₛGₙₛAₙₛGₙₛ^{m}CₙₛAₙₛAₙₛGₙₛAₙₛTₙₒTₙₒAₙₛTₙₛ^{m}Cₙ;
(SEQ ID NO: 37)
AₙₛGₙₛTₙₛTₙₛGₙₛGₙₛAₙₒGₙₛ^{m}CₙₛAₙₛAₙₛGₙₛAₙₛTₙₛTₙₒAₙₛTₙₛ^{m}Cₙ;
(SEQ ID NO: 38)
AₙₛGₙₛTₙₛTₙₛGₙₛGₙₛAₙₛGₙₛ^{m}CₙₒAₙₛAₙₛGₙₛAₙₛTₙₛTₙₒAₙₛTₙₛ^{m}Cₙ;
(SEQ ID NO: 39)
AₙₛGₙₛTₙₛTₙₛGₙₛGₙₛAₙₛGₙₛ^{m}CₙₛAₙₛAₙₒGₙₛAₙₛTₙₛTₙₒAₙₛTₙₛ^{m}Cₙ;
(SEQ ID NO: 40)
AₙₛGₙₛTₙₛTₙₛGₙₛGₙₛAₙₛGₙₛ^{m}CₙₛAₙₛAₙₛGₙₛAₙₒTₙₛTₙₒAₙₛTₙₛ^{m}Cₙ;
(SEQ ID NO: 41)
AₙₛGₙₛTₙₛTₙₛGₙₛGₙₛAₙₛGₙₛ^{m}CₙₒAₙₛAₙₛGₙₛAₙₛTₙₒTₙₛAₙₛTₙₛ^{m}Cₙ;
(SEQ ID NO: 42)
AₙₛGₙₛTₙₛTₙₛGₙₛGₙₛAₙₛGₙₛ^{m}CₙₛAₙₛAₙₛGₙₒAₙₛTₙₒTₙₛAₙₛTₙₛ^{m}Cₙ;
(SEQ ID NO: 43)
AₙₒAₙₛGₙₛTₙₛTₙₛGₙₛGₙₛAₙₛGₙₛ^{m}CₙₛAₙₛAₙₛGₙₛAₙₛTₙₛTₙₛAₙₛTₙₛ^{m}Cₙ
or
(SEQ ID NO: 44)
AₙₒAₙₛGₙₛTₙₛTₙₛGₙₛGₙₛAₙₛGₙₛ^{m}CₙₛAₙₛAₙₛGₙₛAₙₛTₙₛTₙₛAₙₛTₙₛ^{m}Cₙₒ^{m}Cₙ;
wherein:
A=an adenine nucleobase,
^{m}C=a 5-methylcytosine nucleobase,
G=a guanine nucleobase,
T=a thymine nucleobase,
n=a 2′-NMA sugar moiety,
s=a phosphorothioate internucleoside linkage, and
o=a phosphodiester internucleoside linkage.

2. The oligomeric compound of claim 1, consisting of the modified oligonucleotide.

3. The oligomeric compound of claim 1 or claim 2, wherein the modified oligonucleotide is a free acid.

4. The oligomeric compound of claim 1 or claim 2, wherein the modified oligonucleotide is a salt.

5. The oligomeric compound of claim 4, wherein the modified oligonucleotide is a sodium salt or a potassium salt.

6. An oligomeric compound comprising a modified oligonucleotide consisting of 17 to 30 linked nucleosides and having a nucleobase sequence comprising at least 12, at least 13, at least 14, at least 15, at least 16, at least 17, at least 18, at least 19, at least 20, at least 21, at least 22, at least 23, at least 24, or at least 25 consecutive nucleobases of any of the nucleobase sequences of SEQ ID NOs:19-22 or 63-86, wherein the modified oligonucleotide comprises at least one modification selected from a modified sugar moiety and a modified internucleoside linkage.

7. The oligomeric compound of claim 6, wherein the modified oligonucleotide consists of 18-25 linked nucleosides.

8. The oligomeric compound of any of claims 6-7, wherein the modified oligonucleotide consists of 18, 23 or 25 linked nucleosides.

9. The oligomeric compound according to any of claims 6-8, wherein the nucleobase sequence of the modified oligonucleotide comprises the nucleobase sequence of any of SEQ ID NOs: 19-22 or 63-86.

10. The oligomeric compound according to any of claims 6-8, wherein the nucleobase sequence of the modified oligonucleotide consists of the nucleobase sequence of any of SEQ ID Nos: 19-22 or 63-86.

11. The oligomeric compound according to any of claims 6-10, wherein the modified oligonucleotide comprises at least one modified sugar moiety.

12. The oligomeric compound of claim 11, wherein the modified oligonucleotide comprises at least one non-bicyclic modified sugar moiety.

13. The oligomeric compound of claim 12, wherein the non-bicyclic modified sugar moiety is a 2′-MOE sugar moiety or a 2′-NMA sugar moiety.

14. The oligomeric compound of any of claims 11-13, wherein each nucleoside of the modified oligonucleotide comprises a modified sugar moiety.

15. The oligomeric compound of any of claims 11-14, wherein each modified sugar moiety is a 2′-NMA sugar moiety.

16. The oligomeric compound of any of claims 6-15, wherein the modified oligonucleotide comprises at least one modified internucleoside linkage.

17. The oligomeric compound of claim 16, wherein the at least one modified internucleoside linkage is a phosphorothioate internucleoside linkage.

18. The oligomeric compound of claim 16 or claim 17, wherein the modified oligonucleotide comprises at least one phosphodiester internucleoside linkage.

19. The oligomeric compound of any of claims 16-18, wherein each internucleoside linkage is independently selected from a phosphodiester internucleoside linkage and a phosphorothioate internucleoside linkage.

20. The oligomeric compound of any of claims 16, 17, or 19, wherein each internucleoside linkage is a phosphorothioate internucleoside linkage.

21. The oligomeric compound of any of claims 6-20, wherein the modified oligonucleotide comprises at least one modified nucleobase.

22. The oligomeric compound of claim 21, wherein the modified nucleobase is a 5-methylcytosine.

23. An oligomeric compound comprising a modified oligonucleotide according to the following chemical notation:
(SEQ ID NO: 45)
GₙₛGₙₛTₙₒAₙₒGₙₛ^{m}CₙₛAₙₛAₙₛAₙₛAₙₛGₙₛGₙₛGₙₛGₙₛTₙₛAₙₛAₙₛTₙₛAₙₛ^{m}CₙₛAₙₛGₙₛTₙ;
(SEQ ID NO: 46)
GₙₛGₙₛTₙₛAₙₛGₙₛ^{m}CₙₛAₙₛAₙₛAₙₛAₙₛGₙₛGₙₛGₙₛGₙₛTₙₛAₙₛAₙₛTₙₛAₙₛ^{m}CₙₛAₙₛGₙₛTₙ;
(SEQ ID NO: 47)
AₙₛTₙₛ^{m}Cₙₒ^{m}CₙₒAₙₛAₙₒGₙₒTₙₛTₙₒGₙₒGₙₛAₙₛGₙₛ^{m}CₙₛAₙₛAₙₛGₙₛAₙₛTₙₛTₙₛAₙₛTₙₛ^{m}Cₙₛ^{m}CₙₛTₙ;
(SEQ ID NO: 48)
Tₙₛ^{m}Cₙₛ^{m}CₙₒAₙₒAₙₛGₙₒTₙₒTₙₛGₙₒGₙₒAₙₛGₙₛ^{m}CₙₛAₙₛAₙₛGₙₛAₙₛTₙₛTₙₛAₙₛTₙₛ^{m}Cₙₛ^{m}CₙₛTₙₛAₙ;
(SEQ ID NO: 49)
^{m}Cₙₛ^{m}CₙₛAₙₒAₙₒGₙₛTₙₒTₙₒGₙₛGₙₒAₙₒGₙₛ^{m}CₙₛAₙₛAₙₛGₙₛAₙₛTₙₛTₙₛAₙₛTₙₛ^{m}Cₙₛ^{m}CₙₛTₙₛAₙₛTₙ;
(SEQ ID NO: 50)
^{m}CₙₛAₙₛAₙₒGₙₒTₙₛTₙₒGₙₒGₙₛAₙₒGₙₒ^{m}CₙₛAₙₛAₙₛGₙₛAₙₛTₙₛTₙₛAₙₛTₙₛ^{m}Cₙₛ^{m}CₙₛTₙₛAₙₛTₙₛAₙ;
(SEQ ID NO: 51)
AₙₛTₙₛ^{m}Cₙₒ^{m}CₙₒAₙₛAₙₒGₙₒTₙₛTₙₛGₙₛGₙₛAₙₛGₙₛ^{m}CₙₛAₙₛAₙₛGₙₛAₙₛTₙₛTₙₛAₙₛTₙₛ^{m}Cₙₛ^{m}CₙₛTₙ;
(SEQ ID NO: 52)
Tₙₛ^{m}Cₙₛ^{m}CₙₒAₙₒAₙₛGₙₒTₙₒTₙₛGₙₛGₙₛAₙₛGₙₛ^{m}CₙₛAₙₛAₙₛGₙₛAₙₛTₙₛTₙₛAₙₛTₙₛ^{m}Cₙₛ^{m}CₙₛTₙₛAₙ;
(SEQ ID NO: 53)
^{m}Cₙₛ^{m}CₙₛAₙₒAₙₒGₙₛTₙₒTₙₒGₙₛGₙₛAₙₛGₙₛ^{m}CₙₛAₙₛAₙₛGₙₛAₙₛTₙₛTₙₛAₙₛTₙₛ^{m}Cₙₛ^{m}CₙₛTₙₛAₙₛTₙ;
(SEQ ID NO: 54)
^{m}CₙₛAₙₛAₙₒGₙₒTₙₛTₙₒGₙₒGₙₛAₙₛGₙₛ^{m}CₙₛAₙₛAₙₛGₙₛAₙₛTₙₛTₙₛAₙₛTₙₛ^{m}Cₙₛ^{m}CₙₛTₙₛAₙₛTₙₛAₙ;
(SEQ ID NO: 55)
AₙₛTₙₛ^{m}Cₙₒ^{m}CₙₒAₙₛAₙₛGₙₛTₙₛTₙₛGₙₛGₙₛAₙₛGₙₛ^{m}CₙₛAₙₛAₙₛGₙₛAₙₛTₙₛTₙₛAₙₛTₙₛ^{m}Cₙₛ^{m}CₙₛTₙ;
(SEQ ID NO: 56)
Tₙₛ^{m}Cₙₛ^{m}CₙₒAₙₒAₙₛGₙₛTₙₛTₙₛGₙₛGₙₛAₙₛGₙₛ^{m}CₙₛAₙₛAₙₛGₙₛAₙₛTₙₛTₙₛAₙₛTₙₛ^{m}Cₙₛ^{m}CₙₛTₙₛAₙ;
(SEQ ID NO: 57)
^{m}Cₙₛ^{m}CₙₛAₙₒAₙₒGₙₛTₙₛTₙₛGₙₛGₙₛAₙₛGₙₛ^{m}CₙₛAₙₛAₙₛGₙₛAₙₛTₙₛTₙₛAₙₛTₙₛ^{m}Cₙₛ^{m}CₙₛTₙₛAₙₛTₙ;
(SEQ ID NO: 58)
^{m}CₙₛAₙₛAₙₒGₙₒTₙₛTₙₛGₙₛGₙₛAₙₛGₙₛ^{m}CₙₛAₙₛAₙₛGₙₛAₙₛTₙₛTₙₛAₙₛTₙₛ^{m}Cₙₛ^{m}CₙₛTₙₛAₙₛTₙₛAₙ;
(SEQ ID NO: 59)
AₙₛTₙₛ^{m}Cₙₛ^{m}CₙₛAₙₛAₙₛGₙₛTₙₛTₙₛGₙₛGₙₛAₙₛGₙₛ^{m}CₙₛAₙₛAₙₛGₙₛAₙₛTₙₛTₙₛAₙₛTₙₛ^{m}Cₙₛ^{m}CₙₛTₙ;
(SEQ ID NO: 60)
Tₙₛ^{m}Cₙₛ^{m}CₙₛAₙₛAₙₛGₙₛTₙₛTₙₛGₙₛGₙₛAₙₛGₙₛ^{m}CₙₛAₙₛAₙₛGₙₛAₙₛTₙₛTₙₛAₙₛTₙₛ^{m}Cₙₛ^{m}CₙₛTₙₛAₙ;
(SEQ ID NO: 61)
^{m}Cₙₛ^{m}CₙₛAₙₛAₙₛGₙₛTₙₛTₙₛGₙₛGₙₛAₙₛGₙₛ^{m}CₙₛAₙₛAₙₛGₙₛAₙₛTₙₛTₙₛAₙₛTₙₛ^{m}Cₙₛ^{m}CₙₛTₙₛAₙₛTₙ;
or
(SEQ ID NO: 62)
^{m}CₙₛAₙₛAₙₛGₙₛTₙₛTₙₛGₙₛGₙₛAₙₛGₙₛ^{m}CₙₛAₙₛAₙₛGₙₛAₙₛTₙₛTₙₛAₙₛTₙₛ^{m}Cₙₛ^{m}CₙₛTₙₛAₙₛTₙₛAₙ;
wherein:
A=an adenine nucleobase,
^{m}C=a 5-methylcytosine nucleobase,
G=a guanine nucleobase,
T=a thymine nucleobase,
n=a 2′-NMA sugar moiety,
s=a phosphorothioate internucleoside linkage, and
o=a phosphodiester internucleoside linkage.

24. The oligomeric compound of claim 23, consisting of the modified oligonucleotide.

25. The oligomeric compound of claim 23 or claim 24, wherein the modified oligonucleotide is a free acid.

26. The oligomeric compound of claim 23 or claim 24, wherein the modified oligonucleotide is a salt.

27. The oligomeric compound of claim 26, wherein the modified oligonucleotide is a sodium salt or a potassium salt.

28. A population of oligomeric compounds of any of claims 1-27, wherein all of the phosphorothioate internucleoside linkages of the modified oligonucleotide are stereorandom.

29. A pharmaceutical composition comprising an oligomeric compound of any of claims 1-27 or a population of oligomeric compounds of claim 28, and a pharmaceutically acceptable diluent.

30. The pharmaceutical composition of claim 29, wherein the pharmaceutically acceptable diluent is artificial cerebrospinal fluid (aCSF) or PBS.

31. The pharmaceutical composition of claim 29 or claim 30, wherein the pharmaceutical composition consists essentially of the oligomeric compound and aCSF or PBS.

32. The pharmaceutical composition of any of claims 29-31, wherein the pharmaceutical composition consists essentially of the population of oligomeric compounds and aCSF or PBS.

33. A method comprising administering to a subject an oligomeric compound of any of claims 1-27, a population of oligomeric compounds of claim 28, or a pharmaceutical composition of any of claims 29-32.

34. A method of treating a disease associated with SCN1A comprising administering to a subject having a disease associated with SCN1A a therapeutically effective amount of an oligomeric compound of any of claims 1-27, a population of oligomeric compounds of claim 28, or a pharmaceutical composition of any of claims 29-32, thereby treating the disease associated with SCN1A.

35. The method of claim 34, wherein the disease associated with SCN1A is a developmental or epileptic encephalopathic disease.

36. The method of claim 35, wherein the developmental or epileptic encephalopathic disease is Dravet Syndrome.

37. The method of claim 35 or claim 36, wherein the developmental or epileptic encephalopathic disease is any of Genetic Epilepsy with Febrile Seizures Plus (GEFS+), febrile seizures, Idiopathic/Generic Generalized Epilepsies (IGE/GGE), Temporal Lobe Epilepsy, Myoclonic Astatic Epilepsy (MAE), Lennox-Gastaut Syndrome, or Migrating Partial Epilepsy of Infancy (MMPSI).

38. The method of any of claims 33-37, wherein administering the oligomeric compound, the population of oligomeric compounds, or the pharmaceutical composition reduces the frequency of seizures, reduces the duration of seizures, reduces status epilepticus, improves behavioral functions, improves movement and balance, improves orthopedic conditions, improves motor functions, reduces cognitive impairment, improves language and speech, improves visual motor integration functions, improvise visual perception functions, improves executive functions, or reduces dysautonomia.

39. The method of claim 38, wherein the seizures are frequent or prolonged in duration.

40. The method of claim 38 or claim 39, wherein the seizure is any of convulsive, myoclonic, absence, focal, obtundation status, or tonic.

41. The method of any of claims 33-40, wherein the frequency of seizures is reduced.

42. The method of any of claims 33-41, wherein the duration of seizures is reduced.

43. The method of any of claims 33-42, wherein the subject is human.

44. A method of increasing expression of SCN1A in a cell comprising contacting the cell with an oligomeric compound of any of claims 1-27, a population of oligomeric compounds of claim 28, or a pharmaceutical composition of any of claims 29-32.

45. A method of modulating splicing of an SCN1A RNA in a cell comprising contacting the cell with an oligomeric compound of any of claims 1-27.

46. The method of claim 45, wherein the amount of SCN1A RNA that includes an NIE is reduced.

47. The method of claim 45 or claim 46, wherein the amount of SCN1A RNA that includes NIE-1 is reduced.

48. The method of any of claims 45-47, wherein the amount of SCN1A RNA that excludes an NIE is increased.

49. The method of any of claims 45-48, wherein the amount of SCN1A RNA that excludes NIE-1 is increased.

50. The method of any of claims 45-49, wherein the cell is a cerebral cortex, hippocampus, brainstem, or thalamus cell.

51. The method of any of claims 45-50, wherein the cell is a human cell.

52. Use of an oligomeric compound of any of claims 1-27, a population of oligomeric compounds of claim 28, or a pharmaceutical composition of any of claims 29-32 for treating a disease associated with SCN1A.

53. Use of an oligomeric compound of any of claims 1-27 a population of oligomeric compounds of claim 28, or a pharmaceutical composition of any of claims 29-32 in the manufacture of a medicament for treating a disease associated with SCN1A.

54. The use of claim 42 or claim 53, wherein the disease associated with SCN1A is a developmental or epileptic encephalopathic disease.

55. The use of claim 54, wherein the developmental or epileptic encephalopathic disease is Dravet Syndrome.

56. The use of claim 54 or 55, wherein the developmental or epileptic encephalopathic disease is any of Genetic Epilepsy with Febrile Seizures Plus (GEFS+), febrile seizures, Idiopathic/Generic Generalized Epilepsies (IGE/GGE), Temporal Lobe Epilepsy, Myoclonic Astatic Epilepsy (MAE), Lennox-Gastaut Syndrome, or Migrating Partial Epilepsy of Infancy (MMPSI).


SEQUENCE LISTING (89 sequences)

SEQ ID NO: 1 - DNA, 170000 bp, Homo sapiens
  source 1..170000: mol_type = genomic DNA
ttttcattatgaaaagtctgtctacagttttctcttcacatctatctgactacaatatagttataaacccatctgtttcccaagtaacattctcttctaaaagtaggataatttttttccatagaaagctcagatataaaatgaggagactttttgctctttaggaatacaaatcagcttagactaaccaatcacactgaatttttaaagattttaccagtaaccaaaactatatattaatggaaaatggatatgggggaaatacatcctttgtcttaaaaacagaaccccaactttatttaggggtggcaatgggctcagctaaaaacacagtgattccacattcccttgcaggagggagcagccatttttcacagttatgacccataggctataagttaaaatctctagggggtttgggaaagctcttggaggaacctgatttagctggcatgtgtcttatgccttttggtcttcctatccttctccctagcatgtagttaggatgctggaagtgaggcagccatctcaagaccatgaagcaacaaatagaaggatgaaagtcacaagctgaggatgtttgagaggaagaaaaatgagcttaggcctctgaagactgtgaagtcctaccttcagactaatgtcacttaagaaaaacgaacttctattttggttaagttagtatatgtttgattatctcttacaagcagccaaacctataccaagttgatccaattagaagaatgtgccaaaaaaatcttttaaccgtttccaaacgtgcatttcagaagcattcatttactttggcaattattaaactgatatatattagtaaaacatttttatacacttcttaaaatgactcctccaaataactctactaggccatactctatgcacttcctatacagggagggacacattatgaatacttttcatccctccattgattcattcagcaaacacttattggaggcccagaaagcattgtgctaggggcagggatgcaagaatgaaaaacacacacctgattacaaaaggcttagtttatttgaaaagaataagacagaatattttttaaaaacattctattttccagttctttgcttcctgagggttgtttgtttcacatacattagctctccaagggatagagaagcctagcaccataatcaacatcaataaaataaaaggatctgagccaacaaattggtgtacatagcttaatgttcagaacacaaattcatcattgaatatttaattaaatgttgcaccaatgtgttcattactgttgtgagaaataactgaagaaagtttctcattccaatcagtacagaacttgtttttccaaaatcagactactgctaaaagcagatatttagtactactccactttttttttctgtcatttgtatgatataaaaaaatcactgtacatcaagcctttactccagtatcaaaagacaattattcattgcagtatagctgacaaaataacaagttagtgtcaggaaaaccatttttcactcacctcttcctctatgtcaggcatatcatctaacttctttctacctcagtttccttatgagttaagtaatgggttggaattagatcactgtgttccctcatagatctaaattctcttatttcctcactctacttagtgtgatgcattagcagataggtttcccagctaagagcagaacattaagattatttttaaatgatgtgtggttccttctctatttctgttacctcaggaattatacatattagccaacaactcatatcaaaccacaattttatatataattaaacaatataatgtcatcttttattaataatactatatcaccatactcaaaatcagaatagaagactggttttattatttctagcttttaaaagcataacttgcattgcacacatttgggataattgaaagaaaattagatggagtgtacattgttttaaatttgctgttcacattatcccccagatatctaggagtaagtctaacacagattatctcaaaagcacagcaatcctcttgctccattataagaaattattttcagagtctaaaagattgattatatgcaaaatctgaaagctaaaattaagatcttatttttcagtatagacaatagaagtaattaattaacacatctgacatttatcggcaacccaccataggcagagatctgctttcttattagttgcataagattttcagaagttgaaatacattaaatacaaccagtttaacccaccacgttctgaatttgatatcctattaccttgctcacaaacaagatctttaaggaacataaacactctttacaaatcctttagttaccatcctgcaaaacaactcacacccccaataataatctcattacatatatacatatttccctctactcaccacaatgagttaaaggtggacaaaagccctcggtgactgtctttgagttattactttgctttaccatttccttccttttaggtgtcatggtacaggactttactgatagcaattcattcttgttaggtttactgcaaacaccaaaagaggtgtcttgcacttgctggaagacttagcttttatgcttctggtcattcttcccacaaaaagaatgagaccagtttctgagctattcttaactcgccttccaaaatatttcaaaagaggaagacaagaaagacttcatgatttccatgcaaactcccagcctgcctggctgcctgctgctgccaatacttcttgctcttgctggtgatgacattcagagagctccccagcactggtgcttcgttatgtaaggctgtctaggtcaagtgtaggagacacactgctggcctgtggaaactcatggaactgttcctccagattaacacttcaggggttatggaagctggaggaagctgagcttttactacatcttttgggggtaaatatttttgcatgtttcagcagtttatgtactacagtcttggcagatgcctgtcagtcatgctgagaggtgcagggagaaattactgagattacagttctgtcagcatgcttgaataagcatagtaagtcttccagtaatcctggctttgagacacttaaggagtaaataaatcataaattcaaatttgcttatcggacatcatctgtaaccatcaaggaatgaatgttgatttgatatttaaactctgaatacaatttatttgattggaggtgttgtaggagagcattattagaggtggctattagctcaaagagatttactgcagttcttggctgttacgttgcccttgtctctggctgcaaatttgcttcccttgtaagtgagttatatatatgcctgtatttgtcttctgttgtgattgtttgtgatcgtataagaataaactggcagttaggtgacttcaacaaataatgaatcctgcctacttagtttcaaggtgaatactgcagcaaaagcgccattcgacactagcactgaaggagggtggattaactgcattggctcaaactgtgctgtgtttaggagacttgaagggatgattccaggtttgatatgaacagttgttcactctatgggtaaaagtagaaacaatatagcgctgattctgttttagactctcacactgtgtcctgcctcagcaattagttaaaatagcaggctctttattgaatgctgcagaaaagcctagagaggtagcagggtaagattgccgcagcagagtgatatattggccaggaattttaataagatttaaccagatatgataatacaacagatggcatctaatctaaaaaatagatggatatttaaaaagttgaagacaaatgtagcacatttgagagggtttatcttcctgtgtgtccttggcaagtgaagttcagtaccttggacaggatgacatgagttaggaaagtaaagtctgcataaactgtctacgtgatctcaaagagcaaaatggaaagttaactatcaatcatttgttttggtgtctctcagtgatgactggcattaggcaggttacttagcacatactttctaggtaaacaggtagttttaaagtatctaaaatttcttgcattttaaaaattaaattaaaggaatgaaaatgagtgatcgtaaaataaattcccctgaaggtacttttcattctattaaccatattaacagcagcacattgattatttatgatgaaaatataccacttacaattgtatgctgtatttagttgattaaacatgtacatgatttcccaaataatttaaagagacaattatggtccctcggggcctgattttatattactgaagtcaataggcattttccctagacatagcttgagataagagccccatcagaccaggttttactcagtttggcattcagactattcctcataagttaaaaaatgatggatacatacttaattgtacttataatctttgagtttatatatctttcagataacaatgtcagtgttcaaattgtctataattgatttgcatttaatattgtttcttttggctattgctaaaagggattcttctttgttgtaataagcttaaaatatattaattttttcattttaaaatggggaatttgaattaaatgaggtttaaattagttttgattctaccatatatatctttgtattcctctaaatgggaattaacattattaactaatctatgttaaagtgagattacattttttggtgagattatattgtttgaatatttacaatattaagccaaaatttactaagtacctactaaattctaggacagtggttttcaaagtggggtctcctaaccagcaacatcagtactacctgggaacttgctaggaatgcatgttttcgggcctcatgacagacttactgagtcagaagctctagggatgggtcctggcaatctgtgtttgtgtaagcctttcctccaggtgactgtgaggcatgctgaagtctgagaaccactgtgctgggaagtaatttggttaaatattataaaaatggtttttactgtatatagttaaatacatattgtcattaaattttctatgttcatctttgcctagatatcctactgtaagcagtactatgttttaaagatttatgcaattattgagtataccttaagtctaaatatgtccataagaaggtctattttccaacagttccattttagaactttagctcttagaagttaaatactgtaatgggaaaaatactagaggacttatctgtttgttcactgtgtgatttggcacatatcactgaccctctttgatgttcagcttctcatttattaagatgagaggttggcttacatcagggtttctcaactcagtactattgacatttgggtcagatattcttcgctgtggggtctgtcccagacattgtggaattttgagcagggtccttgcctctacccacaaagtgtccatagaaaccctcagttgtgatgaccaaagatatttccagacattgatacctgtctaccaaaggcaaaactgacctcctcctcatttgagaaccactgacctagattagctccgctatgaaaagttccatcataataaataatacaacaaatatatcatttatccttttggaagtaggtaggttaaaacttataaataaatgaagacatgttttacttgtataactcaggtataaggtgggacctgtttgcacccttgaagatctaggtgacgatctattggtagatatctgttggcagatatcttgctcaggtatctaccaataccacagtcagtggaagtccaaaaccttctgcaacttatcagggacttagtcttacatcatcctctgtgtcctggcatcgtggtttcatgtatactacacctaaccactctgtcaagtgacccagtgtctgtctgtccctagtttttaacagagacttacctacaccaatcaccttttcaatttaataaattttattaaagatgtgggcttctacccgcattttctcccttccccatcccatctctgctcctcatcccctccctgagagatctgaacactccaaatttttctcttcttagtatggaaagtagtccatctctccacagctcttatatccagacgtagatgcccaaactcatcaaagagtttagaagataaagggccttcaatttagaaccattcaccatacatagaaggcagttggggtcatatggtataatcataaagttataaaatcataggctgttggaaggaatattgaatttaagttcattttcatgtctttagccttaaaattacaaatgttacaagctagagtctcagaattttaaattataccttcccatgaatttttacctaaaccacttggcaatttagtacagacattttctcgacctgatatgtgcgatagaagtagacaagcctcacacacatgcatgtgcgcctacacacacacacacacacaaatttttctccaacagttaaagattcttttcaatatactattctacaattcaaattaggtgctattctgagatatatgtggattaaggaacctaatataagatataggtagctatatttctgttaaaataataatacctcatacttgaatgactatatatgtattttcaagtaattttaaaaacatgatccctgccgggagtggtggctcatgcctgtaatcccagcacttggggaggccgaagcgggcagatcacgaggtcaggagatcgagaccaggtggctaacacagtgaaatcctgagatcgcaccactgcactccagcctggacgacagagcgagactctgtcccccacaaaaaaaaaaaaaaaaaaaaaaaaaaaaaaattacttaggccaggtgcagtggctcacgcctgtaatcccaacactttgggagaccaaggctggcagatcatgaggtcaggagtttgagaccagcctgaccaacatggtgaaaccccatctctactaaaaaatacaaaaattagctaggcatggtggtgcgctactcagaaggcagaggcaggagaatcgcttgaacccgggaggcggaggttgcagcgagccgagatcacgccactgcactctagcctgggcgacagagtgagactctgtctcaaaaaaaaaaaaaaaaccatgatcctttctgagacccatagcaactctaaaagataggccccattatctctgtcattttgcagtgagaaaataaaggctcaaggtctccaaatgccaaattcacttctcgttccactatcctacaaagcaaaactcaatagttcatgtagtttgaacacattggtaagtataatttctaagtgattttttataaagacctgtctcaagagtcttcctgctctaagaccttcagagagattaccagtgctttgttttattcctaggattaatgcattttacctgacttatcatacatttattcgtatcatatgcttaattaattttccggtatatgtattatagaatgattttaatacatcatttattatactagtcatagtcacatgtgttctatcacttagtgccctcaaaaaactccatgggaataggattattacccccttaagagggaataattcaaacatgagattgaattaaatttccctatacttgagctttaccagaggtaccacactgatgacccagccaacttctctgttttcaccaacgaaaatgcagaacatagcaacagaccatctcttttcactggcaccctccagtgcttgacatatagagagtgtcagtaacttttattgaagaaatactgaaatattgttattgctatgtagttcctttacattttggactttacctaacattaaccaccctagttaattaagtagcaacttgcaaatgaggcctggaagaaccaaaaaacaaattttaaaaagaactgtctgcaactcggaactgctaatgctactttctttgtttcactttatttcacttgagaaaatctctttgggtttgatacggagatgttctcagactttgaatgtgaaaaattgttcccaagatcaaatataaattcctcctttaaaatacaatgtcacaaacctggatcacaaagtcacttccctctttagtcttaaagtgaatactcaagatgaacgtgaatgatcttttattccaatttccaatttagaatattatttcagaaaataattattcaaagttttttgagaaacctccacataaagccaaacaaatttttaccaaagtaaggtaaattctttgtcaactgagagcctgaattagatgattcctcagacttttttctaattataatattctaggtaaaggagtttagctcggcaatatgaccatccaaatatcaaccaaatccaagatgattagatttaatagcgccattttggagatgcattgattattgaagataagtaaaggtctcaacatttttgttttcctcatttaccccaaaatacatccttagtcacacatgtcttaggatctggaatttgtttgataaaaatgctgagttctggccaggcgcagtggctcatgcctgtaatcccagcactttgggaggctgaggtgggcagatcatgaggtcaggagatcgagaccatcctggctaacacagtgaaaccccgtctctattaaaaatacaaaaaattagccgggcgtggtggcaggcgcctgtagtcccagctactcgggaggctgaggcaggagaatggcttgaacccgggaggcggagcttgcagtgagccgagatcctgccaccgcactccagcctgggcgacagagcgaaactccatctcaaaaaaaaaaaaaaaaatgctgagttctgaaaaaataaatctttcttgttttgtttcaaatgaaattatagctctctgataaagtggcattcctttttctgttttatattcaacgagtgtattagtccgtttttgtgctggtaataaagacatacccaagattgggtaatttataaaggaaagaggtttaatggactcacagttccacatgactgggaaggcctcataatcatggcagaagacaaaggaaaagaaaagacacgccttacatggctgcaggcaagagagcttgtgcaggagaactcccatttataaaaccaacagatctcatgaggcttattcacttactagaacagtatgggggaaactgcccccgtgattcaagtatctccacctggcctcacccttgacacatggggattattataattcaaggtgagatttgggtggggacacggccaaaccatatcagcaagtgaaacagtgaaattggatttctaaaagttgtcagtgttctgttggaattattttattcagagaggtaaggaaagtacaacaggtgtcaaccttccatatgaaatttcctaaagaatattattgttacctcatgctgggagaagggaaagtagcagtgatagagtgttttgatttcaaatgtacagcttctagtaaaagcattataattaactttatagtagtaagcacaaccaggaatataaaaatataaaagttttgggtttggcctccagatttaccaaatgttgcttcctaacaattagccaaccctcccacactgacacttactttgtctaaattgagttttaggagaaaacaacccaattataaagacacaactattatcagtcaatgccctgacaagaaacagatggctcctccaaacttgtggaaataaaaagcaaactatttggttttatcttctcaacacgatattttcatggattccttgggaatgcggttccagttttccctctttagcaggtgtgccaggcccttctgatctggctgtctgcctacctctccagactctagccacttccacagacccaactttacatatatcctacatgtatctcctcttcttagaatgcacctcctctttctcctccaccctctttgtgtctactcataaatgcctcagagaaattcctagataagtttaagacagtctagattctacttcccagtcaacaaagaaaaaagtttgataatttctggtgatggaaacagtttcttatgctgttctggaatataaaatggtgccacacctttggagggaaatctattgatacctactgggttaaaaggcacatacccttccacctgacagatccatttcctgagactagatcttaaaggaaaatttgcacaagtgggaaatgcaattgtaaagaaatgtatacatccgcattgtttgaaatagaaaaaaaaaaaagaaaaggaagaaagaaagaaaataacataagtgtttatcatctgagaactgattaaataaactttggtatatccatctatatacagaacaaaatattttaagcaataataagtgaactaattagagataaattatgtaacttaaactaggtttacagccatatatattgtgtgatgtttttctttctttttaaaaaaatatttgtggagatgggttctcagtatgttgcccaggctggtcttgaactcctggccccaagcaatcctcccacctcagcctcccaaattgttgggattacaagtgtgaaacaccatgcctggtgtctggtacaatgttatttataaaaaaaagaaaaatataaaacacgtatatttcagatgcttggatatacacaagaattctaaagaagataaaaaaaaaaaaattaacagcgattgccactgcagactgggcctgagggaagagaaactggaaagttaatttttacttcaagagccactcaagtcaattgacattagaaatgtgacttagaagagtagattgggtttgcttggtgtatctaaatagtatgtacaagcagggctgcaaaggggaatgggaacacccggggagtggtgagcagggcagaatggctgatttacatgttcaaggtgcagaaggaagtgagtaggctttaagagctgaggccagatgtgacagccagggcttctcaaacaccaatgtgcacagaatcacctggggaaatcttgttaaaatgcactttcttatttagaaggtctgggtggagccaaagagtccacattttcaacaagctcccaagaagtggggatgctgccagtatgtggccacactttcagaagcaaggttttaaaattctgaaagtatcctggttttgcgcggtccgtgtcttcacaaacatcaagcagagccgaaaagcacttgtttttagaaaatgagctgagaaaaaaacacctaattaaatacgatgtttgcaacatgaagcttccaccaaaggaagcttactgttaagtgttcacgatcagcagttctgctttgctagctttttcatttctaacaaatttagacttctggagattgaagctttgccagattgaggcatgcttctcccctcactactcatgtgtttagtgtatttgttaacttggtgagagtacgtccaaacatagttatattctttcacttgctatggaagattgaagaatctgagacaagtaagaaggaagatctccatttgcctccctcccaccctgccacatgctgccccctaacatgtttgacaagaatccaggctacagactgatcatacacctaaaaactatcttcggtcagctacttgtagaaatgtctctcccccattatgtccttatatgagcttaatgtgatgtaatccttagcagtaaagaaagactattgtcttctgtttgtttgcctgtgaactcgaattgcagagactttccaccataattttaataatgtgatattgtggagagttttagtatggccttatggtaagattaggtactttggagatacactgctggtttcagaatcacacttagtcacctcgttactgtgtgatcttggccaagatgctcaactactctgagcctcagttgcctcatttataaattcatgaagataacatggtaatagcaactattttatgagactgttgtgaggtttgaaggtcccaaaatatattaagtcctcaataaaattaacaacaattattattattctaaaattattcttagaatttatctaaaaatagtactgcttgaatatccatatgtttattcctgagtctctacctgctatgatttggctgtgccccactcaaatctcatcttgaattgtagctcccataattcccatgtgttgtgggagggacctggtgagagataactgaatcgtgggagcagttttccccatactgttcttgtggtggtgtataagtctcacgagatggttttataaggggtttcccctttcacttggctctcattctctcttgcctgctgccatgtaagatgtgcctttcatcttctgccatgattacccagtctcaggtatgtctttatcagcagcatgaaaatgaactaatacactgggcaatttacaaaagaaaaacaagtttcttgaactcatagttccacgtggctggggaggcctcacaatcatgctggaggcaaggagtagcaagtcacatctcacatggatggcagcaggcaaaaagagagtttttgcaggcaaactcccatttttaaagccatcagatcttgtgagacttattcactatcatgagaacagcacaggaaaaacccacccacatgattcaattacctcccactgggttcctcccatgacacatgggaatggtgggagttacaattcaagatgagatttcagtggggacacagccaaaccatatcattccactcctggcccctcccaaatctcatgtcctcacatttcaaaaccaatcttgccttcccaacagtcctccaaagtcttaactcatttcagcattaactgaaaagtccacaatccaaagtctcaaggcaagtcccttctgcctatgaggctataaaatcaatagcaagttagttacctcctagatacaatgggagtacaggcattgggcaaatacagccattcaaaatgggagaaattggccaaaacagaggaactacagaccctgtgcaagtctgaaatccagcagggcagtcaaatctttaagctccaaaatgatctttgattccatgtctcacatccaagtcacgttgatgcaagaggtgggttcccatggtcttgggaagctccacacctgttgctttgcagggtacagactccctcctagttgctttcacaggctggcattaagtgtggcttttccaggaacacagtgcaagctgtcagtggatctaccattctggggtctggaggatggtgaccctcttctcatagctccaccaagtggtacactagtagggaatctgtgtgggggctctgaccccacatttcccttccatactgccttaacagaggttctccatgagggccccacccctgcagcaaacttttgctgggcatccaggcgtttccatacatcttctgaaatctaggcagaggttctcaaaccccagtttttgacttctgtgcacttgccggctcaacaccacgtgggagctgccaaggcttgaggcttgcaccctctgaagctatgacccatgcactatgttggcccctttcagccatagctggaatggctgaggcaaagggcacaaagtccctaggctgcatacagcatgggaactctgggcctggcccatgaaaccacattttcatcctaggcctccaggcttgtgatgggaggagctgctatgaaggcctctgaatgccctggagacattttcttcattgtcttggggattaacattcagatcctcattgcttatgcaaatttctgcagctagcttgactttctcctcagaaaatgggattttcttttctattgcattgtcaggctgcaaattttccaaacttttatgctctgtttcccttttaaaattgaatgcctttaatagcacccaagtcaccccttgaatgctttgccacttagaaatttcttccaccacataccctaaatcatctctcccaagttcaaagttccacaaatgtctagggcagggacaaaatgccaccagtctctttgctaaaatataacaagagtcacctttgctccagttcccaacaagttattcatctccatctgagatcacctcagcctggatttcgttgttcatatcattatcagcattttggtcaaagccattcaacaagtctctagggagttccaaactttcccacattttcctttcttcttctgagccctccaaattgttccaacctctgcctgttacccagttccaaagtggcttccacatttttgggaatctttttagcagtgccccactcccagtaccaatttactgagtagtccattttcaagctgctgataatgacatacctgagacatgacaatttacaaaagaaagaggtttattgaacttacagttccaaatggctggggaggcttcacaatcatggcagaaggcaacgaggagcaagtcacatctcacatggatggcagcaggcaaaaagagagctgtgtcagcaaaatcccatttttaaagccatcatatctgatgagacttatgcactatcatgagaacaacacaggaaagactcgccaccatgattcaattacctcccaccaggttcctcctactacatatgggaattgtgggagttacaattcaagatgagatttgagtggggacacagccaaaccatatcatacactaccatatactaggcactgtattaagggttagggtattaaatagaatcttttatcataaagatttttgttcatctctgtaaaatattttagacacatgtgttttgctttttaaacaaagaaaaagagatgtttttagagataagatgagacacattgttattttccccagttcccaccagcccttcacaactgccaacctggaaaaaacattcccagagcccaacaaatgctggagccctggaagaagggctactagcaggagcactggactccaaggaatgcagaccagggaagagagtgttgggagtattaaaatgtaggttagaagtggaataatggacaagtagagggaaagactttcaagtccactttccagggattgagaaacttggggtccttcatccgttggctttgaatgcagcccaagacaggcattagcagaaaattggagagtctccccgaccacacctcacttcctagcttaaaaaagcagctgcattcctctttagttaaacctctcagttgggtcttctaaagctctagctctcaccggaatctggtgccactttcactcttgcctggaatggcattggctctctctattgctagttcctgggcactcatcaaccatcttaacccacttggtcaatagtttctccataattatttttattttaaccattttgaatgtaccatctactttctgctaggaccatgactgcatctgaggtataattttaaagcaacaaatggttcatggaagggtaactctgtttgtgatgcccaagagaggagacttagggcagatagagatacatataaagctactgctgtcctagagagcagagggacaagaaaaggggagagagaacactcattagcagttgtgtgtgtgtagcataacataacatatatatatatgcatatatatgatatgtaatcttagaaacaactaccatgtgaagtagagcaaatgatcgctgagtttcatccttgctaagagcatgggaaaatccacattctatgtttgcttagttgcagaaagaataagggaagcagaaacgtcaggggtcacatcccctatgtgtcaattaaaggggtgcagactcagaaggtgtccttacctactttatttagcctgctcttccccacaaagatgaggaatacaggtgactttgatgtcatgatagcaatcagaatgtaaaagagactttcaaaaagacctagtgcaacatggaaacaaggactttagtctctgagcttccttatgtatcaagtgtcatagaaaatgttgagacaaagtggttctgtcatttcaacaacccataatgagcagaagaatctgtttgcctttcatgccactgcagatgaacaggtatgagagactgcacttaggcatgaaagtgccgctgcctagcacctctatcccatgctcacatccgaatcttctcaggtgtgggtaagacatctcagttctctgctatttgatatgaataggggcaatcccagaagcagcaactgagctcatgggccccaaaatttccaaggaatcacgaggagaactaggctaaatgtcattattttctgaccttcgatgtaccttttccctagtcacttgccaatctcttctaaagggcctcagtccctggagtggaccctaatgatttctgtccattatttggcctagagtagaattcttgcttttctcaaggaaagaaacaaaaagaaaagatgtaaccaaatgtaatttatgtgagtttgtaaattgatatccgaagattggaactgcatacagatgtgtcaaaaatgttaatttccccaagaaatatggtagcacctaattctagcgacacttgtatactttacagatggctttgctgtgctcctggtaaagtcaatgactttctgcctttgaacatgaaaggaaaaagatgttgacaggaattctttagggaaacagaaactacatatttgcttatgctaagttatggcaaagttcagttttattctgtaaataaagcgttgtcttcataaatatcagctatatcctttagagtaaagaaagtcctctttattaaaaaaatctaattgaaactcttcaataggtaggcaagtgaggtgattgatataaagatatacttaagtggaaaattggtattcgagttccaataagtaatcaatcatattgttcttggtaatagcttttgttgattcatctttgcaagggatgtcggggggtaggggggattgctgaatgaaataaatcaggaaagaacccaagcattatgtttaactgacttaagagcaaaggaaagaacacataaacacagaatagaatccacacagtaacttcattctttatgattgctcaggcattttcattatttgcatttatgtttattttctctttatcctcctgtggcaatagatatggagataatgctttaaatattgtgatgtaccttgatcttttctgaagaaccaaagctttatgaaatcttgatacatttaaaggaggggtataaagctcaggcatgtgaaatgttgaattttgtctccccatatgacagataaccatcatataaatgtggtttattagactaaaaaagtagaacacatatttttagattacatcttggcatttcaaagaaagtatagaacttcaagatagtttgcttcctggatattttctggagctgtctttgggcctgcattcctgaaagctgtcaatgctacactctgcatacccagagctcgtgcccttgggcttgagtactttgtggttaatatttaagaaccgcatgtggagctagattgaaggacagtcactttctttattctttgctaaaaccaatgttggttagtccatcaccttcttaaacagtaactgcattcatacccttcatcacatccatctctctgtcaaaacatgagtgaaaatcaattgtggcatatctagaaatacttcactcacttccattttgcttcttatgtgctgatgtaccttactcaatattaatttcctttagagttaaactgtgggaaaacttgaatggacctttgtggtataaaactatctatgtgaccttggacaagtcactgcacctctcttggcatcagtttcctcatatccaaaataagaacacttgcctggtttatctctgagacttttccagctccacaatttctcaatgattagcaaaaccctatcgataaagcaatggtaatgatttatgtaaattgacattcttttttatgaggtttacaaagttattttattctactccagggttttgttggaagtctattacggaatttttattcatcaatactcagcagatgttatcaggttctctccagaaactaccttaattctaccacagacttgggatatgtttggggaacaatgacaaaaaaaataagtcatagttcttactttcaaagagattattgagtggggtcagaccgtggagaggtgcaccgatagagggtggaggaagatgcggtcaaagaagagaaggtcctgaaaacagaggaaacaaaggaagcggaacagtgaaggcataatggctccctgcttgtttaggaaaaggcagagtttgttgttttgaaaaacgacatttagtgtcatcttcactaaatattaataatgtcttcttacacgttagctaaagaagaaaagatcaaaaataaaccaggcccccaaataataaaggatttaaaattaaagatatatacagagcactatagtaatttttgaagtgctgttcagttggtgatataatcagaggaataatgctattacatcaagtgataaaaagtaattttgtgatttaatactcattctgaacctatattgtaatgctaatctcatcagatgattgacattcatatacttaatttccctttgagaaatcaggttcatgtattattctgtcttgtttataaaagctacttatttggtttaggaatattgttatttccctctccaacaccaatgtctgcctatgtaattgaaaggaaattgcagtgaatcttccaacaaggagtaactgcttccctgaaataatcacctgcagacataattctggcttctgtgcgttgtctcgatagcagggaaggcagagggcaactggggaatctgattatctccatcataatcccacaacttccctcctttctcttttgcttttaattactgcattgtggtgagttaagaaaagcctatactattttttcttctccactaccagaaagagagagagataactggcattctctctctaaggatttttaaaatacttttaataaaattttaaatagtgtaatgtgtgcccacaagcacattggttctaaagatacttcaaaaatacttcagaatcaaatctgtaagaaaaaaaaattcaattatttatgttcattatattacaagtgatttaaaaactggttatctgtgcatcaaaaaatgcaagcctatagcagctctgattcagtgaattccagttgcggggaggggaggagccctcagatatttttatatcatatttgaaaaaaatggctgattattatttttttaatttatggaatgtatggactttaccagagtttcaaaagtccactttagtacctataagatattttgaaatcagttggccaattcttgccatcaaaaaattgtaactttaaacttgcagaaaaactagttggctctagtcctaaaagagagttcttaattgcagtttctgcatttatttgtctaatagcaaataaatttcacattcatttctgactccctttcaaaagggcaggttaatctcaaaaggaaatatatgagtttatcattttcggctaggatatggtttctatttctaaaatagtggcacagtgttggtgaatgctttgtgagagcaaggaattggtagtagatttttgcagaatgctgcatttaccaaaagaaaaaaaaaaaccactaggaaattgacatctacaaaagcaaagtacttaagtagtaaattacatttaccaaggatcacatcagtagtgtgctttatctgattattccaatcaaaagggggcaaagtcaggatttgtagtgatacagtggtcctgtagatctaaaatcttaaggtctgagtttaaatccaaactctagcatttgctatctgtgttgggatcactccccgacttcatcagaagacctgctaacatctagacttgtctgttgtcaccaccatctcctttgggataccacctcaaggtactcaagttcgcctccagcctccaattcctttcctgcggtgggaagagaggggagacccagcttcatgttgctcagtgactatagattcccaaacaaatgacaggggaatttagaaacatttctgccttaaaactcaaatgtcatttataactctaaacaagggaatctggaactcaaaacataatgatttggtcatcactttgggaaatttttggctaaaaattaagaatggtttccttctccttggcaggcataaagttaattaataaattctcttcataaggcagctggttaaagtttcagaactccacaactccaggagatacacatttgaaagaatatttttgataaccaattacctttattttctatgcaagaaaatattaaagaatattcagatactaattacactaattcctcagccttctgataatgagatagttctctctttgaactctttcaagatctaatggcatcgagagactctcctttatgtcgtaaaactaacagacaaaattacctggcccagctttttccacctgagtgacctcacgctctctttaatacccaaaatctattgacagagattatgtcccattataatattcccttttatgttttggcactgagcagtgcaactgacagagctataacaaagtaacaaccacattcacaggacagagacggagtcttacagagacggagtcttacaaagactaaatgacatcttacgccttcattctcttttccttgttaggaactctcataaaaaagccacagccatgccccctcagcctgtttagcaggttcatgcattacagattgaagtattgtcacctaggaagtaggcagctaattttttccagcttctgaaacaagattacaaaggggttcctggaatggctaactttaccaacgggggcagaggaagattagtttttcattttttgaagttggaaacgtaacagtgagaaagagacaaaattatctctcaataaattaatctacaaaaaaataaataaaatgcaaagctggttccacagggagtttacattctgggagccaaataagatacatatataaaactcttagaacaaggtcaaatgggataagtgtagtagtgacatagggtagcacagagttttgttttaatatcttctgctaactttaaaaaataaatatgtgattttaagtgcatattacactaatttaataaaaatctcatattttgcctttatgctcagtctgaaacatggaagatttaaagttactaatactgtaaaactgcttcagctctgacgaatgaagtatctgtgtacagaatgatgagttgttagttaatagtactctttgggccatgcttgggatattggccttttctaggagtctgtatgagaatagtcatttatctccttatgctcaatgctttcaacttgtcaaatgaccatgtttcataagatacaacataaaattgttctataattaacaaaagaaaaaagaattctatctgctgagaaaatagaaaagacgttggagaagagagaatatttgacccagacatttaaaatatccgccgaatttgctatgtgtagatggatgaaaggtctgccaagagaagtgaaggttgtgaacaaagacatggaggcggaagaaaggtagtgtgcacactgaatgcgttgtgcctttccatatgactgacatgtcaatgtaaactccacaaagggagggttttgatctggtttgtttgccgatgttacttaagtatctggaaaagtgcctggcatatatcaggtgcatgaagaagtgaataaatgaagtgtcagatgcccagaatatgaataatacttgatagatgcagtactggcaactttcttgaagctaatgaagtggttaggactccatagttcaacagaaaccaagttcacttaagcaaacatggagtttgagtttatcatgggactcaacatcaagaaagtgtttggatcttgagtagactggagatggaaacagtctgtctctgtcttttgtcacttcttcctccacatctgtgtggctggctgctatttcagtatcctggtccacctggtcctgagtccccatggtgagggaaagctgactccatagctgccagaatcacatgtagtggctctctttgaccaggtcagctgcgtagataccctctcttgctccagtcctctgtagcaagagggtaggtttaccactaaggatcaggggagaagaactattgggggaattgttaagagcagacactcagaaaaattggcacctataatatgcaattagaattcattaaaacatgtggccaggtatgttcttattttgtgtttttacatatctaatctctaagtactattcatatggaatgtattattctaaaaaaattgcctgttagttcatgcagttcccactctcttttcatctctaacatcttggcaatcgtttaccagttgttgatcacctgtctcctcatcttctttcaactttgccctgactctaccagatactgggcaacatcatttcccagacattgagtaacttcaaatgaatgtgttcatgctactccaactggcttttgggaaagcactggatagagtttttgagctggaacaggaaattatcttacttaatttctttcttttatagataaaaagatggatccacaagatggacccattggtccataattaagggctagtaaatgttttgtaaacatagaaatcaaaattacttttctgtgattctagaacaggaaaaaatggtgaacttttaggtatgttgcaaagaacagtggattattctttgggctcttgggaccaacagccttagttttattgctttgcaagctatgtggtcttgaataaacctcagtaaggctcagaaagcttcagtgttctcaagtataaataggaataattataatataataacattaatagtatttatctcaccatcctgtaataaggaagagaaggattaaatattgaaggttatattgataaatgataaaacacatgcaatgattgtaataagtgtgttagatggcaaaagaaaaataatgaggatgaaatatttacttccaatgactaatctacacataatcgtgattttatgtatgactgaatgtttaaggctgattccattttagaatacaatatataacataaaaagctctactctcagttccaagagttataattagtttggcatggatttttattgtcttagaaaaaactgatattcatggtttttataattctatctttgggtaataggcaattggactctctttctatatatatttgcttattaaaatgtgttattataattaagctttaggctgtgtccacaagatgggaaaacattggagtaaaatatagtaaaaccatagaaattgagagtgggttgaaagtcttgtttaatctgtaattacagatgaagatagtaagaatgtaaatgtccatagaagacttcagtggctttgttaaaggcatgtgttcatcagtgttgaagtaaaactcacattttgtgactctgagctgttcactataatatgaaacctaatcctaaaacacaatattaaaacagaaaaatgtgaagccatagatctgtaataaagggccacctgattgtttcctggtggaataaaaagacagttttatttataagatatatcatttttatagataacaatgattcatattaggaataaagtaacagtttcaaactaaaataatgctaaagtttttcaagtactacttgaaaatagctatatttactttcaaaccttttcctctttgagtcattaggttcatgatattatatagcaatagggaatgaaagagaagcaaggagaagcaatactgggagattacagagaagaaaggaaaaaaggctgagagaaaagaggttgaggaagaaatcataaatctggattgtgagaaagtgtttaatatttagccactagatggcgatgtaatgtaaggtgctgtcttgactttttttttttttttttgaaacaagctatttgctgatttgtattaggtaccatagagtgaggcgaggatgaagccgagaggatactgcagaggtctctggtgcatgtgtgtatgtgtgcgtttgtgtgtgtttgtgtgtctgtgtgttctgccccagtgagactgcagcccttgtaaatactttgacaccttttgcaagaaggaatctgaacaattgcaactgaaggcacattgttatcatctcgtctttgggtgatgctgttcctcactgcagatggataattttccttttaatcaggtaagccatctaattgtttcatcttgattttaagtttattcattccagttattcctttggaaaaagagtccatggaaattcagtttgggcagagcaggaagtccatttttgtatgtgtattcagaccaactgtccccctcctccctctcctcctcttctcctcccctcccctcccccctcctctctcaaccttccatgaactgaaatcaggtttgttttgcagttcagcattttgatagaagatgggattctttggcctgaaatagcttggcatctggccagttcttgcctttgcttgccttcatcctattcaaggtcccttttgatcctgttgaagccccctccttcactggtgctttggaagtggactagctggctgggaatagaggtcatattgttcttaaggacggagcaaaaattagatctcagttttaactccacaggaagaaagaaggcagcggggctgctgctggagcctagcattgcaggggtattcagcttcctgaggctgaggctgggcttttgagaagaaatgcctcatgccttgaacaaggaaggggcaatgatttccgccccccacctctgccaactcctattgagccacactaaaaaagagcttgagcagagtggtaaagagtccccttttctatccaatgccaaataggaaacattgacttacttttacttagacagaggtcacaaccagcaatgatactgcagatactgctcagttcctagtgtgaaccgtcttgcttttcagaacagcataagaattatttctgagtggaggtgaggcttgtccaaatgtctttgctatcatggatttcctgactcctacctgtttgaggtatgtgtttaaagacccgacccacttgctgcatttacttctcatttctttttgttctctttgattactcactggtgattgttaacaaatcggtctggagtggtgggtatgtgcttttcacctgtccaatgcaacaattaagtcaaggcagggtatattactgtcaatatgtgcatgtgactaaatatttctgagtgcagtagcaaacttgcaaattgccaattgctgttgctatcacacctataaaatgtgtcttcttcttctatggatttacaggttcacgtggtatttctgatgcgctagtatagtaacagaacatatttctccaaaaagcactttaaatcatcccaagaggatgttgccagacagaaataggaaaattccatgctgaggttggtgattcctgctttagtttctcctgcccagagaagtgggtagcttacctttcctatttgctgcaatacctggcacgccagggtgatgcaactgggagcttctgcacgttcgcggggcatctgacattcttctttccctcagctgtgcatttcaggagaatgactgtggtgaaaaactcttttaaaactctcttttatgattgttgcttttgtaattgcaatgaagcaattacaaaatcacatctctcttctaggaggagttttgcaatttaggccttccttcacaaagctcctggaagagatttttagctggtgtctattttaggaacataatgtgtagacaggaatgaatggccacagagatttacgggaaaccaatttcttggagagggtgaggtaacattataaaccactagaacagagcacaagtggctggttatatcacatggacccagcaatggaattagttttcattaaactcactgtaactcatttgtgtggctgtttttcctccttccttcacattgtttgtctgttttgtttcaaatatctatataggaggccaggatacccagagtgttcatactggtaacaataacacattggattttgctgccaatactaatagacttggtttacatgccccctcttgctgggaaatgtgctgtctggagagtcatttattatggtaatttctctttaagacatggcaggagggattgtccatctatgggctggggcaaggcggggcacaattggaggcctagttaattatacagtattcaagaaggcactaaggagctcagtcaggattctgttgtaccaaccagagataccaggggcaaccagaccgactagatacccaaagtaaactctatcaccaccaacagaggaatccagtaagaaacattctagttccttgtatagagggtactatcagggaatccagacaatcagagagggtaaacacaagagcacctcatctgggaattgctgacgaagaagagaaaatctggggcttgcttggtgttacctctaggacgtcaccaaccaggcatttctcaagggtttatgtcggtgtgagcctgacagtgacacacaaaagcatccctccaaaatttagcctggggaccttgaaaggtattgctttctccctgggcagttggagaggctgaacctactattttctccctgtactcgccaaagggcacaaagcctactctcttccagctacctcagaatggggagggtgggagaaacagatcccagagaaggatgggagagaatcagcaattgaaggtcctgtatagtcaccctcagggtcaatggggctgtgctccgtgaggttatcacttcctttttaaattctaattagtgaatgaagcatgttccaccgctcaattgagattgcattagtttagggtagggtgggaagaaatttattctcattgcacttgtgatcagaaaaataagttgtgacagaattccccaagctgccccaaagtttgcttttccaacttttaaattttgtccacattcctagatttccatttattaattgttttatttatcaaaaacatatagcatttactctgtgccagccactatcctaagcagtactttgcaaatatgatctcatttaatcctatgaggtagatactattgttaattttacaggtgacaaaaccaaggcacagtgaggttcaattaacttgctgggttatacagttagtaagtggcagaatccaagcagtccagccacaccgtggccatggcgtctgtgttcttgatgtttgctcatcatccatgtgatggtagagctacagcttagccaagagactcaggactgaagttgggtgcagtcgtacatgtgagctgtaattcatctgctttctgacatgccaaaattccaaaaacagtttggtttagaatgattacccaaatccactccatttgtttgtttgtttgtttgttgtttttttgagacagagtctcactctgtcacccaggctgcagtgcagtggcacgttcttggctcactgcaatttctgcctcccgggttgaagtgattctcctgcctcagcctccccagtagctgggactacaggcgagtgtcaccatgcctggctaatttttgtattttgtagagatggggtttcactatgttggcccggctagtctcgatctcctgacctcaagtgatctgcctgcctcagcctcccaaagtgctgggattacaggagtgagccatcacaccgggctatttgttaaaaaattaaaaaaaaaaaagctctcctgtacaatagaagatagaagggtctgcgttctaagtgcaggaatgtggtgaataaagcaaaattcctacccacatgcagctttcagtctggtgatagagttggacagtaggcaaatcaatgtgcaaagaaatgtcagtaaatgtaagttctttgaagaaaaatactgcaagataaagagatagtgaggatgagaggggcatgcaaacagaaaataatgaaagaggtggagaaaaagtttttttaagcagaaatctaatgaggccaatgatggattgaatcaaaggtgctaaagggtgaagtgcctcacaggtatgaagagtggcacaaaccttgtgggtgatcctgaaagtaaaagaaacgaacagaaatagcaccatagaggtgtgcagccagatctgtggaatctcagagattgggcttagggctctaactgcccccgacttttaaaaaaacaaatcaaagagttatgtcaaataaatttaaaacattcaatgttaacaagttattaggtttaaaaaaaacataaatcagtcattttcacaaaaaaaagttttgagagtttgtaaagttctctgaaaaaatgttgcagttcctagaatgcaaaaaaattgcctcaaaaatgttgaaaagaactgatccaaaggtagtgagttatctcagttgattgttcacagtcagttacagatcaaactccttgttctacgctttcccctcttctcactactgctcttgtctaaaaacttttttaaaaattaaaaaatattgaaaaggtactatttgatgaaaaaaaaattgtttgaatgagatcattggttagaatgatatagtcacccagtcagtttttcttcctccagaaatgatacactgaccaggtgcaggcaagaaagagacactaagcatgaaaaccagtgtttacatcccccatagtggccaggacacaggagctgcttggagggatttgaacaaatctttttttttttttttttttttttttttttttttttttttgcaaatgaatgagtaggaatgtgatttcaaaataatttgataaacatcagctttcattttcatctatgttaaaacttttgttaccatatttttattccttcaagactcatattccaatggtttatctttttatatttttttcactatttgcttcccctcattctcagttacctatggacactgtagttcttctctagaaaatgtgaatttgcttctcttctaacttagaatcaacctgacattttagcttttctctgtaaatggtcaaaagtctttgccttatcatattgcctgtttctcctttggtgctctacctacaaacttagatttaattttgatgaagaatctactctaacttttttcttttgtcacttgtgcttttagtctcatgtataagaaaccgttgcctaatctgaggtcataaaaatttacttctattatttttcaactagttgtataattttagctcttgcatttagatctaggattcattctgagtcaactttttgcatataatgtgaggtaggggttcagcattactcttttgtttgttcatatctagttgtcccagcaccacgtgttgaaaagacaattctttccccggtgaattgtcttattaacattgttgtccatgcgtttatgattttagcatcaattatttcttacagtgggtactagggatatatcttttaagaaaaggtcagtgagattctttgttcttgagaatccacataactttaaagaatcagaaaactgatccatatacccttaactattaaacactttattgatctaacttcacaacaaaatgtttcttctcattattggaaacatattcatttgtgactccactgaaaatttaaggagataatgagcaaatatttccagggaaaggttatatttattccaaagaaacatatttactgtattgcacatgctttcccaaagttacacaaatattatccatgttctttgtaatatatgttgctagtatgcttttgtttcacagataccattggggaaactactgaacttgagatagaggtgtgccattcaaatagggttggtttctgtattagttaacttgggctaccatgacaaagtaccacagacagggtggcttaaataacagtatttatcacagttctggaggctcgaagtccaagaacaaggtactgggaagtttggtgtcttctaaagcccgtctccttggcttgctgatgtctgtcttttcctctgtgcatgtgcattcctggtgtctcttttctgtctaaatttcatcttcttttaaggacacaagtcatattggattgtggttcaccctaaaagtctcatttcactaaaccacttcttcaaaggctctaactcctaacacagtcacattctgaggtaccggagtttgagactccaacatatggatttggggaggatacaattcagcctctaacagttgtctctggcaatacttatgactactaaaatcataagcaaagaaggttaaattgatatttttttgttgttgatgaaactgttgtggttattttataaatgatggattttcattattcataccctcctcctactggataataatgagttatctgtgtatgtgttaagattctaataaaggcaattaattaggtttgtaattacaggcatgatgactaacaaagtgggggcagaaataaaatgatggtgacactgattccactgctgtgcctataatgtcacatttacattcatgatctgtgataagaattaagatgctcatttatttcagtgaggaaagtgacagaaaaggatataaaattaggaattcatggggaattggtaaaaaaaaattaaagtattacaataagtaactgaaactgactcaggttaatttaagtataaaagcaatgtcttcaaaagatattatagttcacagagttaagggatagctacaatctagactgaaaaatgccaggagcagagaaaactataagagcagatagcccagttggtagacttttggtggtttctgtaaggtgctgtaatctagaatgaatcagctcaactgctttcaggctctttgtctttctttctttttctttttttttttttttcttttttttcctgacattcatgtttaacaccaaaattccaggaagtaaacatctggttggtcaagtttgggtcatgaatctgatccttttagttaagagtaaacttaactaacagttccttgactgacatttccaccaaactatccccatataggaaaactaattcaacccaaacaaaattggagagattaatagtctacataggccagatacagtggctcaaacctgtaattccagcactttggaaggccaaggtgggcagatcacctgaggtcaggagtttgggaccagcctggccaacatggtgaaaccccgtctctactaaaaatacaaaaaaattagccaggcatggtggtgtgcacctgcagtcccagctactcaggaggctgagccaggagagtcacttgaacccgggaggtggaggttgcagcaagccgagatcatgccactgcactccggcctggcgtctggatgacagagcaagactctgtctcaaaaaaaaaaaaaaaaaaaaaaaaaaaagagaaaagaaaaaagaaaacagtctacatacacaatgctggaaggttgaaagaatggctaaataaatcatagtctaggcaaatgatgaaatattatagtaccattaaacataatgtttcaatgaatatttaattctatataaaattgcccaggttataatagtatattaaaaagtaccttacaaacatatatatctaggatggccccaattacggaaaaaaaacaaacatgtttttccttagaaaaagccctgaagataatataatagttatctctggtttatagaattaggatttctgttttcttctttataaagtttttgttttaaaaatagaatattacatcttattttttcttataataaaagtaatacataaaggaaaaggaacataaagtacataaaaaaggaacataaagtgcataaaacaaaaaaaaaatttaaatagaatattacatcttatttttcttatgacaaaagtaatacataaagcaaaagtaatacataaagcaaaagggaaatataaattaataaaatgatgaaataactcacaattttacacaacaaaattattattgttaataatttattttttcaaatgctttggtattgtttcttaccaaacaatttggcattgttttggtatgttttcttaaaataaagttcaaaataaacatatgttgccttaaaaactagaataaaagtgtacattagttttcaaatagaatttagtaatctatttcatgcaaagatactcattgtatctttaatcaaaatagtaaatttagtttaaattattgatgaaacaattgaatgtttgaatattttgtcttttggaatattaataaaactaggccacaaaccttgtataataaaagtacagtattttgaaaattttgtaacaatggaaaagctttgggaatcggtctaatataaaattagacttgaaatagcaaattggtggtgttctgaaaatcatgttacttggtaacctaagttttctctttagaactcagcacgaattttaagaaatttatcttatatttataaaacatctactatttgcaatgagttgtttttagcaaaacttgacctcaaattgaacatttgagaatactgaagaataaataagaagcacctgtaaatactgacatagtatgtccttaaatttttaaactacttagaaccaaattcatcaagttggcaaaatgaagttactagattatcaaatggaatggcaagtgaaaatgagccagaggtggcagaatggagtaagaagggtaattattagggaatgctgcatttggagatgtgaaattatatgtgttgaggatgtgaggaaagaaatctgtgtgaggagaaatagattattaatgatgtaacttagtaacatcagattaagggtccccaaaccctgggccacggaccagtactggtccctggcctgttaggaactgggtcgcacagtaggaggtgagcagtgggccagggagcattacagcctgagctccgcctcctgtcagatcagtggcggcattagattctcataggagcacaaaccctattgtgaactgtgcatgcacaggatctaggttgtgtgctccttatgagattctaatgccagcccccactccccacaatgtccgtggaacaactgtcttccacaaaaccagtccctggtgccaaaaaggttggggactccttatctagattattgtaaagcaaatattcataattttaagattaaagaaaaaatccaataaagatgagaaaccggatctcagagtttccaattacatattcatagaggaaagaaagctaagagaattctgtcacatacatatttcaatctaagaaattcagtggatttgaaaatcctggaaagagttactttgagacttcacaaatgctaccaggcagcagttttgatattaccctggaatgtgatatgccacttaaaaaggccagtagtattttgggctatgtatgtcagtgtgttgggacaaaaccaaggagaaaacagtccggtgtcaggtctagaataccatcatttcagtgccacctgggcctcagcatttcaggtttcaggtgatttgaggagtctgtgaggtcgcacaataatatttaaggcctttaaagggtcaatcaacttgtacaaatgaaggcaagagtgcctttggaagacattccaaaaaggaagtttgagcatactcaagaagaatctagccactgcactccagcctgcgcaagagagtgagacgctttctcaaaaaaaaaaaaaaaaaaaaaaaaaaaagaaactaaatagaaatcagtaagcaaagaaaaagaaaataggagtgtgatttttcttcctgaaatctctgtggaatagcaaaaagtggaatcttgactgtatcttctctgggaccaaactttatcttactctccctgactctctctctctctctctcttttttttttcattcctaggcattttgttttactctgtttttgcatactctgctgatttctaaccctccttccattcagtaacatataaaaaaaacttttcacagtcatgttctcagaatgttcatagtctttaatgcatatagaatataatccaaaaaatcaaaaaaaaaatttttttttttgagacagagtctcactctattgcccaggctggagtgcaatggcaggatctcggctcactgcaatctccgcctcctgggttcaagcgattctcctgcctcagcctcctgagtagctgggattacaggcgcacactaccacacccagctaatttttctatgttggccaggctgaactcctgatctcgtgatccgcctgccttggcctcccaaagtgctgggattacaggcatgaacctccgtgcccggccaaaaaattcttaatgtgattttagcagttattttcaaaatgtggtccacagatgctcttgcattcccttgagactttttaaatgattcatcaggtccaaactattttcataatgatacaaaaacattatttacctttttcactgtgttgacatttggaagaacaatgaaacccctagccttaacacttatcaagacagtgacaacaaactatactgtcattgtattcttcactgtaattcattcccagtaaaaataagtaaataaataaatgaataaaattaaagccagttgcactaaaagaatgtctttgacgaaacactagaaattaattttatttaatctataccattaagtacacatatttttaatattcagtatacatgaagaaatggaaagtatacatgattaacatctgctgtatatcaacaataatgctggttgtcttaaggtaaagcacttgtgtagttgtttgagttgcaagctaaactagctgtacttccatagaatactatttttatttgaaaaaatgactgagagacaaattgtgattattcaaacagatattgaaaagacaaatttttgaaaataaatggagactgtcattttaaaaatacatttgacaatatttgttgtcagtgacaaaatttggcctttcaagagaaaattattattttgaaaaagatgcatctgccaccatgagctcaacagcttctcaaaacttacagggtttctgatgtgattagtggtaatattaacagatgaataatgaaatcttccaatattttgaagacctgcataacttagccaagcactcatcccattagatctttttcttttagaaatatggttattttcacttgaaatttattttgttgacatgtgatttatgattataatttataaatttattaattgatgtgcttgtgatttttctgctttctaacatggtaaatggcaatacattgaactcattataaagaagaattttttgtttcctcaataatattaacaatcaaaaggagtcttgagacaaaaaaattatgacaaggatgaatttgataattcatcagattctgggacactgaatctgatgaatttgataattcatcagattctgggacactgaatctgatgaattatcagattatctgataaattattcagattaagtttgttgcactgtattagttgagattattataatgcagataattgagtcccccagaattatgcaggatagagaagatatttgtcacattacacaagttagtacatttttcattactgtaatatgagctgcacatatcagtttagttcagtaagttctgtataaagtgagtaaaaattgttgcaaagcaaatcagaggattataaaatataaatgttctaatactcttgttaggactttcatctttcctcacctaaaccaatgtaagaagtttctattggtctttttctaaatgttcacccactcccttccctcaattagaacctgatctccatactaccaccagtgacctttcaaacatgtgggccacatctctactttttaagactgtaagttatatcctggagtaaaagctattcagttattcagcatggctggctttcatgagctgtccaggtttatttcttaccccatggtcgatctaccttaccattagtccttcagaaagaactaattgcatttctgtgaagacctaaagctgtttctatctctaggtctttgctactgcttctttatttggtgaatagccacgacctcttcttccccaagtatgattaatttccttttgtgttgtgcctaggtcctgtatacatttctatcatctctcccactgcactttattgtgcttattcccctgtcttcctctgctatactgtaccttctttgcaatatggtgcagcatattgtctgatgtgactaagtctcattgccactgcctacttccgttttcggtgtctaggttctcaatagaaattgttaaatgaaaatgaaacaaaacaaaaacatttttagggttacctttaatattaatacctgacagttttacattttataggatgctttcgcaaatactgtttttgtagaaatatgcttgaagcatataataatttaaataattcttttatttcttatgcaagtcccaaactcctattctaaactcatatatactatgagtattgaactacttatatatactatgattaagcactgcttttcaaagtaattctgaatgcattttttcttcaaattattactgttagctgtttttacttattcagtgtctgagaaatttgattacttctccttttatcattttgaggtattcttttttgtttgtttgttttagacggagtttcgctcttgtttcccaggctgctggagtgcaatgaagcaatctcggctcactgcaacctccacttcctgggttcaagcaattctcctgtctcagccccccaagtggcggggactacaagcatgcgccaccatgcccagctaattttttgtatttttagtagagatggggtttcatcatgttgaccaggctggtcttgaactcctgacctcaggtgatccacctgtttcgaccttccaaagttacaggcgtgagctaccgtgcctagctcattttgaggcattctttggtctttgcttgagtctaattattagcaaattattcaagtactatattttatagttatggaatgcattgatattatagaaaacagaatatttagaaaagcataaagaagaaaataatatttgtgatctcataatctatgtgattttccccacaataataaaatattgcttttaacatgttaatattttgctctaaaccatttctcttcctgtttgaataatgggattcctatggtgttttcaattttgtagcacatattacatcttaaacattttttaatgccatcagaatagttcaaaagtataatattaatggcttcataatatttccttgtagttttacatttatttaattattctgataatcatggaaattttatttcattttagttgcatcattacttttgaagtaaatttaaataacacattaaaaataccaagaattttacatttttattaatagccaaaagatcatctgttgtataaacagatcccaaaacttagggttttcttgattaaggaaaacagaaggccaacaaagtccatgcagtacacacaagatatagtcaattaacacttaaatctgaaggaaacaattccagtgcatggtttcttgcactgggcttgttctctttctttttgattctcccttctggtcctcactgggagtaggaatcacacaaagagcttggctcactactttcattcactctctccttctctcatcatttgttcaaaatgaacccatttgggctagttctttaatactctcagatgttttcctgctttcagaaacaggcacaaaataatttatatattaacattgaacaactctctgaaaatcattgcattgtttaactaatttctgatttttctaccactttacagcagaaaacaactttcttcttagagttcttgatacaaactttttaagaaatttaaaatatacattaaaatctgtctttgatgagcatactaaataatatgatagctggaagggaggtaaggaagtggtaacagttttaaagttcttatttatttttcaaatatgaatagttacaggtttgttattatctaagaaagagaattattaatattctccatgagatccaaggatcacattatatctcaaaccaaggtaacattagagtgtgaaaaacaattttaattattattttatcaattttaaaaatttttgtatatttgtggggtacaagtgcaattttgctacattgatatattgcattttggtgaagttacggccttcagtgcatccatcactggagcaatgtgcattgtatccaccaagcaacctcctatcatccacccaccttgcacctactacccttccaagtctccattgtgttttatcttaaagtgtagtaattcttgccctctagcaataaccttggtgtatgcatctaattctcagagggtaatctacaatttcaaaactttaaaagtagctgtttaaaattatagcacacctgttattttatatttatgattagtatatatatcggttgttcaagctaaaagacaaatgacaggaaacattttaatcaatagcattcttttagctattatggtaatattcatatttattgggacttagagtaaaacagtcaggaagttggtcagagcaagaaataattttctcattcttttttcatttactagtcattcatttaacaatatgtattgagaacctagtatatttaatactaggtgccaagatatctactcttaagtacttagattctaaatttaaaaaaaagccaagtactactgttttaaccacaccacttctgacatcaaatatgtgggtcgttttgtttttgtttttgtttctgtttttccctcaccactcaattgtctgacagccagttgagtgtcccacaattcaattcaataataacttggcattatcctcagataccacaagtcaatggctcagtctcacaaaactgccccacttcaaacatcagttgcaagacccaggcctctggaacttctgactgaccagctataaatcctccagttcaataatgtgctacaacagctcacagaatttaagagcttactatttccagtttattgtaaaggaacagcctggaagagacatatagcagaaagtatagggtaggagaagctatccaaactccataatttagagtttttatggaggtctcattatgtaggcataattgattaaatcattgaccattggtgattagcttaatcttcaacccctagcccatcccttgcagatgcaggtctttctagtgactagcctcaatcctgaagctatgtaggggccctcagccacttgtcatctcattagcatacaaaaaggcactccagagattctaaagattatagaagctcttatgtcagaaaatggaactaaggccaaatataagaaagatgctgctatcaccaatattaatcaggaaataacaagggttataggagctctgtgccaggcagcaagaactaagaccaaatacaggcacacctcagagatattacacatttggttccagcccaccacaaaaaagccaatattgcaataaagtgagtcacacaagtgttttgatttcccagtgcatataaaagttatgtttacaccatactatagtctattaggtatgtgatagtattatgttgaaaaaaagtgcataccttaatttaaaaatgctttattgctaaaaaatgctaacagtcatctgagccttcagtgagtcatataatctttttgctggtggagagtctcgctttgatgttgatggatgctgactgatcagggtggtagttgctgaaggttgagatgactgtgacaatttcttacaataaaacaacaatgaagtttgccacgttgattgatcttcctttcacaaaagattcctctgtagcatgtgatgctgtttgatagcattttacccacagtataacttgtttcaaaattggagtcaatgctttcaagccctgccactgctttatcaaataaatttatgtaatatctaaatccttttttgtcatttcaacaatgtcctcagcatcttcaccaggagtaggttctatttcaagaaatcactttctttgctcgtctataagaagcaactcctcctctcttaaggttttatcatgacattgcagcaattcagtcacatcttcaggctgcattctaattctagttctcttgctatttctgccacatttacagttacttcttccactgaatcttgtacccctcaaagtcatccatgaaagttggaatcgatttcttccaaactcctgttcatatggatatttttacctcctcccatgaattacaaatgttcttaatgggatctaggatggtgaattttatccagatgattttcaatttactttgctcagatccatcagaggaatcactctctatagcagctatagtcttataaaatgtatgtcttaaataataagacttgaaaataaaaattattcctcaatccatgagctgcagagtgcgtgttgtgctatcaggcatgaaaataacattaatctccttgtacatctccatcagagctctttgcaacattgtcagtcagcggtaatattttgaatggaatcttttttttttttctgagcagtaggtctcaatagcaggcttaaaacattcaataaagcatgctgtaaacagatgtgctgccattcaggctttgctgttctacttatagagcacaggcagagtatatttagcatacttcttaagggccctaggatttttttgactagtaaatgaaaattggcttcaacttaaagtcactggtgacattagctcctaacaagagactgaacctattctgtgaagctttaaagccaagcattgacttctcctctctagctatgaaagtcctagatggcatcttcttttaatataaggctgtttcatctatattgaaaatctgttgtttagtgtagtcaccttcatcaatgattgtattagtctgttctcaccctactaataaagacatacccgagactggctaatttataaaggaaagcagtttaacggactcacagttccacatggctagggaggcctcaaacaatcatggtggaagatgaaggaggagcaaagggatgttttacatggtggcaggcaaagaaagtttgtgtagagcaactcccctttataaaaccatcagatctcctgagacttattcactaccacaagatcagcatgagaaggacctgcccccatgattcaattacctcccactgggttcctcccatgacatgtgggaattatgggagctacaattcaagatgagatttgggtggggacatagccaaaccatatcaatgatcttagctagatcttctggataacttgctgcagcttctacaccagtacctactgcttaaccttgcactttaattttatggaggcagcttctttccttaaacctcataaaccaaactctgctaacttcaaacttttcttctgtagctttcttacctctctcagccttcacagagttcaagagagttagagccttgctctgaattaggctttggctcaagggaatgttatggctggttcaatcttttgttcagactttcttcatatcagcaataaagccattttactttcttatcatttttgtgttcactggagtagcacttttaatttccttcaagaatatttaagaatatttctttttcattcacaactcggctaactgtttggttcatgaggcctagcatttggcctatctaggtttcaacatgtcttcctcacaaagcttaattatttctagcttttaatttaaagtgagaaacattcaattcttcctttgacttgaacacttaaaggccattgtagggattaaaaaaaaattttttttatttgtatggctacatagtaggtgtatacctttatgggttacatgagatgttttgatacaggcgtatcatatgcaataatcacatgagagtaaatggggtatgtgtcccctcaagcatttatcattactttgtgttacaaacattccaattatactcttttagctatttttcaatatagaataaattattgttgactgtgataactgttgtgttactagatcttattctatttaactacatttctggatcttattctatctaactacatttttatacccattaatcattcccacctccccaccacccacatagtagggttattgattggaataatttcaatattcttgtgtctcaaggaactgggaagcccaaggagaggaagagagacgggaacagcatgttggtggagcagtcagagcagacgtaacaccaattaagttcactcttatttgggtgcagttcttgccatccccaagcaattactaaagtgacattaaatattactgatcacaggtcaccataacagatataataatagtgaaaaagtttgaaatattgcaagaattaccaaaatgtgacacagatacacgaagtgagcacatgctgttggaaaaatggcaccaaaattgacttgctggatgtagggttgccacaaactttcaatttgtgaaaaactgtatctgtgaagcacaataaagcaaagtacaatatgtcaaggtatgtatgtatatacttcttatgataagaccacaccaagtatataaaatattttctaaagatggcacttgttacttctttgagactctcaatgtgttgcatactatgtttaagcttcaaattactgatcttaaggaaatcaatgttcttatctatctgtactgttatcttgatgtaaattcagtagtgtacctagttagtcagagtaggatccaaaattccaacctaaattttgaaacaaagatttttctttattataacgcccaacataacaccaagagtagttaataaacatactaagatgaacaattattcttgactttaggcaaaaagaaaattactaaaagatgtatttattaatcaaatgctgtaatttttgttattatttctgtaaagttctaaactccttgaaatcctatagaagagaaaagaataaaaaattgtcaaggcattctgattcaaacagaaaaagaagggaatcaaataattatcactgcttacatgattatcattgtagttttagctccttgttagttcagtgcttttttgtatatttatagagttgtgcagcaatcattacaatctcattttagaacatttccattgagctaaaaagaaacttgatgcccacttgtagtcactctctattcccactcccagtcctaggcaatcactatttgctgtctctgtacatttgcttattctagacattttatatgaatagaatcatgcaatgtgtcatcttttgtgactttcttctttcacttaacataatatctttgagattcactcacctaacagcatgtttcagtattcattcattttaattgaccaatcatactcccttatatggatataccacattttgtttatccatttaccagctaatggacatttgggtgctttccacttttgggctattgttaataaggctgctatgaacatgtccatataagtcttcatatggatacatgttttcatttaccttgattagatacctaggaatgaaattactgggtcatataatgaatctatgtttaacattgtaagacactgccagtgtatttccaaatggctgtaccattttacattcctactaacaatatatgagggttccagtttctctatatccttcccaacacttgtgattatctgttttttttattattattatagtcattccagtgtgtgtgacgtgatgtctcattgggttttttatttgcatttccttcatgattaatgatgttgagcatctgatgtgctcgttgattagtcatttatcttctttggacaagtatctacccaaatcttctgcctatttttatagttaggttctttggggttttttattattgacttgtaagagttttaaaatatatatatatatgccagatacaatcgcttatcagatatatgatttgcaaatacgttctttcaatctgtgggttgtcttcattttttgatgttgtcttttgaagggcaaatgattttttatgaagtacaatttatcaatgttctcttttgaccacttatgcatttggtgttgcagccaagaaattattaccttatgtatgtgcaccatatttactcatatttttaaagaattatagttttagttcttacgtttaaatttatgattaattttgaattaatctttgtacattgttatccacagtttcaaatccttccttttgcatgtggatatctggtttttctagtactagttgctgaaatgattattctttcccaattaaattatgttagcacccaaagctgaattgataaaaattgttagccaagttgtgttctcttcttatttattcaagaatggttcagaataagaactattgctactttacatgtgtcagatatctaaatattaagaaaagtatgtgatcactataaaattcatactttccagtaaattacatttaccatacaatgttcaatactgtaacatattctgtattttaacttttgatttttttttataactgttgaattttgaactatcgtgttttgtaacctctcactcttaggtgttgttattccaaatattttattaattaattcagcaactattgtgtgactgttatgggtaaagtactttgcacgatacagtgaagaatgcaaaggtaaatgctgctctcaaggtgcttatggactaaagcaaaaaacaagacatattcacaagcaaatatactgggaggtagaagtcagtcgtcaagtaacaccagagttgcagataaatgcttatgtgaattcgaagattacttgctttttgtttcagaaaagtgctagtggatgaagagatcaacagcctatatgacagcaaatttggttccatgttggtctatacaagtagttaagggcaaagaataagcagtaagtttctgatgaatataaataaaatgtatgtagctgttaagaagtatagtctgtgattttaaaaaaacatgaataatgatattcatgtacaagacatgacagttaccagtgtattgcctcttagctctctttggtagctaaccagaaatagtgtattgggcacttaaagcatttttcttagaccgtgagcataacgttactctttgtcagtagagggcagtgaagggtcactgcaggaggaaggggcttctcttcctggttctaatgctacattttgattttacttcgatcattagcaatgtggaccattagcaatggaccattggcaacgtgcttgtgtaatgacatctggtggtgtttttccccagccacatgcccagatggggcagtctgttggtgacttcacagcctctatataagggcctagtgaccactttgccacagttctcccagcactgacaccgcacactgccaacacagacacatcctcacttcagtgccgcactccttctgaaggcccacccaccaaccttagctcacctgtgccctaaagaattgtttctgcttgcccaacagtagcccaactgtggaccagagctccggccaggacaccccagcaaactattctgctgtagagaaagagggcttccaccacacttttcccaatgaagtttggaccccaaccttgggatgggaaatccttccagttcactcttctttgagtcctctaccttagctccaggataccctttagattctcttacatctttatggttactctcctatcagtacttaataattcttcatatattaaactttctctgtttaaattagcatgtggtttctgtatctttttttgtttgtttttgtttttgttttgagacggagtctcgctctatcgcccaggctggagcacagtggcgcaatctgggctcactgcaagctccgctttccaggttcacgccattctcctgccttagcctcctgagtagctgggactacaggcacttgccacccagcccggctcattttttgtatttttagtagagacgggttttcaccgtgttagccaggatggtctcgatctcctgacctcgtgatccacccgcctcggcctcccaaagtgctgggattacaggcgtgagccaccgcgcccggcccggtttctgtatcttgattgtgtcccagactgatacctgttgccacaagttattttgctcagttaatattactgattttcaaatgcttcagtttataggggtaaagacaatttcttgatgctatccctgaaaaggaattggacaagtttaacttatgtcatcttctcatgggcttcatggaaaatagcctttttcactagtgaatattggatatgctctagcatttccactgagaaataaaaattaggcattcattcaaaaaagtgtgtttgatcctatttctaattcaaatgctttgattattattgagttattcatatagtagattttctaaggattaattgtttttggccaagataaaccaaatgtggtagatgtaaactattttcatatggtcaaatacgtgatgattattttaatataaagggtttcactgaagcataactacaatgtaaagggtttcactgaagcataactacatatgtagaagttttaatgtgtatgaggtctgtaattcataaaattgttggttgattctttctgtttcacttctattggcaaaggtttaatttacattcaggcctggtggtaaccctgttaataaccaaattcattctctcattttaaacttataacttggcaaaagggaaacccttgatgaagttgtttttaaacttttatgaatgattctttttaacctttttaaactctgaaaccttttaaaagtgaaaatattcatcactttctaaagagtatccactttgtaaggtccaggtacagcaagcaaatatgtataaaacattaaattaagaaaacttcacactttttcctctatagcaaaaatcctaaggagtaagacatatgatataagctatttcccctaggacttcactgaggttatttgaaacatatcagaagctgagaaattacgtcatttcttttgctatttttattccttgaggaaataacttttttcaggaccatgataccaattcttttcttctttacaagcattcctgtgctttcttacaacagcatactggcaacacaaagtagccaaatcggctctaagacaaatgtctgtgaaggctgttggctgagaaatatcatttggcggtctcatagtctcatatataaaatgtaccttaatttttttatttttttgagacaagatctggctctgtcacgcaggttggagtgcagtggtgccaccttggctcatgcagcctccacttcccaggctcaagcaatccccccacctcagccttccaagtagctggaactacacaaacgcaccaccatgcctggttaatttttgtattttttgtagagacggggttttgccatgttgcccaggctggtgtcgagcttgtgagctcaagcaatacacctgcctcagccttccaaagtgatgggattacagatgtgagccaccatgcccagccccatttactttttaaaccaccacaatcattattatatcgtagcactctaggttcacagaggtgattagggacaactatgttggtaagtcaatgaactctcttctgttcttaccttactagattatgctgtaacattagacaatgttcatcattatctctttcttactctctcttccctgttacttcttctagaaaatgggtctttgtttgtattctgcaaagattaaagttgaattctaacaatgaaagcattcattttgactaaactctagttccataatttttcttagaaagtattggcaaatctgtttatgtctcttttcaattatttttcctactatctctatataccaccaataaaattaactaactaaatttagttttatttcaaatcactcagtggttcttttattcaaaagatattaataagcatagtgtcaggcactatgccatgaacagagatacatttccaggaaattgatataatggaaagaaactagggctagagtcagacaaatcagctctagctatacactgctctagctatacacagtatcactttaattagctacattttttctgacttttattgcttcatctgtaaaatagtgataataatatatattttcacgttgtatggtaaagtctaaagttatgttcaccaatacataatgcagttcttggcactgggtagatgcttttaaacactattagtgttctttatggtattttttctaagtgactaaaagatcatattaattttacatgcctcacatgtcctcagtcctttacctttatcttcctggtgaatttccttttattttacaaaacccatttcagctctcacctacttaggaagcttctcttaacatttttccccctcctcatatcccaaaagaagtgattatatcctcttctgtgcttcctccgtgtctcaaagataacttattgttatacttcctcagtatttatttatttatttatttatttatttatttatttatttatttaagacagagtcttgctttgtcacccaggctggagtgcagcggtgcgaactcggctcactgcaacctccacctcccgggttcaagtgattctcctgcctcagtctcccgagtagctgggaccacaggtgcctgccaccatgcccggctaaattttgtgttttcagtaaagaccaggttttgccatgttggccagactggtcttgagctcctggcctcaggtgatccgccctccttggcttcccaaagtgctgggattacaggcgtgagccaccgcgcccagcctacttcctcaatattataatatattgctattcaactgtgtctggcctaacagactgtctgctcctctagaacagaaaccgtcatatatatatatatatatctcccaaatctaaagtcaggcttgaaatctaataggcagctcaaaaacaatttattcttaaacagaattgaacaatgccttaaggtacaaaagttgtgcattatccacaaaatagctcgaatattactgaaactttgttgaatggtattttctctgaagcacccatgttgcttcaaaggacatgcttttgttcttatttatggctgtgtaatgttccctagcatatatgtaccacattttctttatccagtcagccattaatggacatctaggttgattgcatgccattgctattgtgaatagtgctgcaatgaacatatgcgtgcatgtgtctttttggtagaatgatttatagtcctttggatatatacccagtattgggattgctgggtcaaatggtagttctgttttcatttattctgagaaatcttcaaactgccttccacagtggctgaactaatatccattctcaccaccagtgtataagtgttcccttttccccacaaccttgccaacatctgttattttctgactttttttttttcttttttttcttttttttttttttgagatgtaatcttactctgtcgcccaggctggaatggagtgctgtggagcgatctcggctcactccaagctccgcctcctgggttcacgccattctcctgcctcagcctcctgagtagctgggactacaggcgcccaccaccacacccggctaattttttgtatttttagtagaaatggggtttcaccgtgttagtcaggatggtctcgatatcctgacctcgtgatccgcccgcctcaacctcccaaagtgctgggattgcaggcgcgagtcaccgcgccccgcccatgactttttaaaaattgtcattctgactggtgtgagatggtatctcattgtggttttgattttcatttctctaatgattcatgatgttgagcattttttcatatgcttgttggtcacgtgtatgtattcttttgagaactgtctttttaattctttgcccatttttaaatggggttgtttttcacttgttgatttatgtaagttccttatagatactggatattagaccttcatcatatgcatggtttgcaaatattttctcccattctgctggttgtctgtttactctattgatagttttgtttttgttgtgcagatgctctttattttaattaggtcccacttgtctatttttggttttgttgcaattgcttttggagactttgtcatgaaatctttgccaaggccttaatcgaggtatttcttagttttcttctagggtttagagttttaggtcttacatttaagtctttaatccatcttgaactgatttttgtatatggtgaaaggaaggggtccagtttcaatcttctgcgtatggttagccagttatcccagcacagtttatttaacagggagtcttttctgccttgcttgctattatcgattgtgttcaagatcagaacgtcgtatgtgtgtgactttatttctgggttctctcttctgtttcattggtctatgtgtctgtttttttttttttttaattatactttaagttttagggtacatgtgcacattgtgcaggttagttacatatgtatacatgtgccatgctggtgcgctgcacccactaactcgtcatccagcattaggtatatctcccaatgctatccctcccccctccccccaccccaccacagtccccagagtgtgatattccccttcctgtgtccatgtgatctcattgttcaattcccacctatgagtgagaatatgcggtgtttggttttttgttcttgcgatagtttacagagaatgatgatttccaattgcatccatgtccctacaaaggacatgaactcatcattttttatggctgcatagtattccatagtgtatatgtgccacattttcttaatccagtctatcattgttggacatttgggttggttccaagtctttgctattgtgaataatgccgcaataaacatacgtgtgcatgtgtctttatagcagcatgatttatagtcctttgggtatatacccagtaatgggatggctgggtcaaatggtatttctagttctagatccctgaggaatcgccacactgacttccacaatggttgaactagtttacagtcccaccaacagtgtaaaagtgttcctatttctccacatcctctccagcacctgttgtttcctgactttttaatgattgccattctaactggtgtgagatggtatctcattgtggttttgatttgcatttctctgatggccagtgatgatgagcattttttcatgtgttttttggctgcataaatgtcttcttttgagaagtgtctgttcatgtcctttgcccactttttgatggggttgtttttttcttgtaaatttgtttgagttcattgtagattctggatattagccctttgtcagatgagtaggttgtgaaaattttctcccattttgtaggttgcctgttcactctgatggtagttcttttgctgtgcagaagctctttagtttaattagatcccatttgtcaattttgtcttttgttgccattgcttttggtgttttggacatgaggtccttgcccatgcctatgtcctgaatggtaatgcctaggttttcttctagggtttttatggttttaggtctaacgtttaaatctttaatccatcttgaattgatttttgtataaggtgtaaggaagggatccagtttcagctttctacatatggctagccagttttcccagcaccatttattaaatagggaatcctttccccattgcttgtttttctcaggtttgtcaaagatcagatggttgtagatatgtggcgttatttctgagggctctgttctgttccattgatctatatctctgttttggtaccagtaccatgctgtttgggttactgtagccttgtagtatagtttgaagtcaggtagtgtgatgcctccagctttgttcttttggcttaggattgccttggcgatgcgggctcttttttggttccatatgaactttaaagtagttttttccaattctgtgaagaaaggcattggtagcttgatggggatggcattgaatctgtaaattaccttgggcagtatggccattttcacgatattgattcttcccacccatgagcatggaatgttcttccatttgtttgtatcctcttttatttccttgagcagtggtttgtagttctccttgaagaggtccttcacatcccttgtaagttggattcctaggtattttattctctttgaagcaattgtgaatgggagttcactcatgatttggctctctgtttgtctgttgttggtgtataagaatgcttgtgatttttgtacattgattttgtatcctgagactttgctgaagttgcttatcagcttaaggagattttgggctgagacaatggggttttctagatatacaatcatgtcgtctgcaaacagggacaatttgacttcctcttttcctaattgaataccctttatttccttctcctgcctaattgccctggccagaacttccaacactatgttgaataggagtggtgagagagggcatccctgccttgtgcccgttttcaaagggaatgcttccagtttttgcccattcagtatgatattggctgtgggtttgtcatagatagctcttattattttgagatacgtcccatcaatacctaatttattgagagtttttagcatgaagggttgttgaattttgtcaaaggctttttctgcatctattgagataatcatgtggtttttgtctttggctctgtttatatgctggattacatttattgatttgcgtatattgaaccagccttgcatcccagggatgaagcccacttgattgtggtggataagctgtttttgtgccagtactatgctgttttggttactgtagacttgtattatagttgggtggtgtgatgcctcagacattgttctttttgtttaggattgctttggctagtcaggatctttttgggttttatatgaattttcaaatagtttttctaattctgtgaaaaatgacattggtagtttgatagaaatagcattgaatctgtaaattgctttgggtggcatagtcattttaacaatattaattcttcctacccatgagcatggaatgtttttctatttgtgtgtgccatctctgatttctttcatcagtgtttcataattcttgttgtagagatctttcatctccctggttagctgtgttactaggtattttatacttttcgtggctatcgtgaatgagattgcattattgacttggctctcagcttggatgttattggtgtatggaaatgctactgatttttgtacattaatttttcatcctgaaactttcctgaagttgtttatttgatctaggagcctttgggaagagactgtggggttttgttttctaggtataaaatcatatcattttcaaagagagatagtttgacttcctctcttcgtatttggatgccttttatttctttctcctgcctggttgctctggctaggacttccagcattatgttgagtaggagtggtaagagtgagcacccttgtttcgtttcagttcttagggagaatgctttcatcttttgcctcctcagtatgatgttggctgtgagttggttatagatggctcttatgctttggcctaatttgttgagggtttttaacatgaagagatgttgaattttattgaaagccttttccgcatcttaattaagatgatcatgtggttttttgtttttagttctgtttatgtgatgaatcacatctattgatatgtatatgttgaaccaaccttgcatcctaggaataaagcctacttgatcatggtggattagctttttggatgtgctgctgggttcaatttgctagtattttgttgaggatttttgtgtctatattcatcagggatgttagcttaaagttttcttttttcattgtgtctctgccagattttgatagacagatagatatttcttgtgaattgttacaaatgcaagagaagaaaacttcatccaaacaaggcagattgcatacaaatatttctacaatgacctctatttatctagataaaagttttctaattctacaagactctagaatacaacttcaaatagaggctaggtgtgatagctcatgcctgtaatcccagaaatttgggagaccaaggtggaaggattgcttgaagctagtagttcaagatccacctaggcagcaaagtgagaccctgtccctacaaaatatttttaaaaattagctggtcaggtgatacacatctgtactccaagctactgaggaggctgaggaagggatcacttgagcccagtagttcaaggctgcagtgagctatgatcatgccactgcacgccagcctgggtgacagggcaagaccctatttttttttaattgaatacaagtacctttgaacataccaaatataattttatatctcttatttcaaagtgaagtctagataatctcacgtataagcatttttttaaacatatagcagatctctagagatggtgtttatctgattttcaaaccctggcatttagaaaaaacaaattaataaagactaaaggatgaggtaggcattgagatttctagaactattaataggtttccccttctaggaaggaaggtgaaaaaagtttcaaaaatagaaacgaagtgtaatcactgtacatttgtaaattgttaaaaaagaaaacattggaaggccaaggcgggcagattgcttgagctcacgagttcaagaccagcctgggtgacatggtggaaccctgtgtctactaaaatacaaaaaattagccaggtgtggtggcatgcacctgtagtcccagctacttgggaggctaagccaggagaattgcttgaacctgggagagttaggttgcagtgagccgcgatcatgccactgcactacagcccgggcaacagagcctgaccccgtctccaaaaacacaaaaacaaaaacaaaaaaaacacacacaaagaagaaagaagtatatagtattttagatttacatcactaatggtgaacacttagagtgttttgttaagtctagtggtaaactttatgaaagggcaaatttggaggtctagatttcagaaaagtgattgaagaactttaggactttcatcttaactcagtatcttcaaatttcaataaaaacaaaacaaaaacaacaaaaagtcacacatatattaaaattaatagtcacctagaatagcatagaataaaattactatgaccttgtgaaaagaaattgtcagatcaatgtaattttcttttgagttgaagcaaaatgtcatgttgatcaagaaaaaaataatggataccacttatctgtgctttaagaaggtatttgcatatctatacatgtatatgttcttataaaaaacatgatctctcaggctgggcgcagtggctcacgcctgtaatcccagcactttgggaggccgaggtgggtggatcacgaggtcaggagttcaagaccagcctggccaacatggtgaaaccctgtctctactaaaaatacaaaaattagctgggtgtggtggtgggtgcctgtaatcccagctactcgggaagctgaagcagagaattgtttgaaccagggaggcggagcttgcagtgagccgaattcacaccactgcactccagcctgggcgacagagcgagactctgtaacaacaaccaaaaaaaaaagtgatttttcatccctgatttccaactttcatatcagaattatggactaccatatgaatgaaagaaattgaactttcagtgtcagttgatagtaacaatagacattgactctcaaaagtcttctgtggagtcaataactctttgagagaatttgcattttattaaatacttacatttgtataatttaaaaagaatatagtgtgaatgtttgtaagacatgtatttaacatgaatccaaaactggagtgggacattatgaattcttgaaaactttagaataaatctgggcctaattttctgcaattattcatctgtatcatattgcttttaggggagggttcaagtggttcaaggtaagtaatgatgattttatgacatcctggaaaaatatattaattaccatttatctaaattaatctaaagactattaccatttaatagtttttcatgctgggtattgacattaaatataaattttaagtgaaactgatattaatagctctgaggaatgtataaataggaaacagaataaccttgaaacctttaggaattggcccatcaaggcaagtgagactctagtgtcaagtttggttagcgtgtagtatattttgaaccaagaacaaactatacaaataacaggatggcagagaagctaattagtcacaaatttggtagaaccaataaggctcttttatttaacaggcatcgttgtttctgattgatagcaggaagaacaggagaatcatgggatcatgggatatttttatatttaaagatcagttttattgtttcaatccccctttgttaaaatgtcaaactggcttgtacaatgacaaaggctaaataaaggtattatattactaaaaaacttgacacagtaattcgtactaagggaaatgtatacaaaaatcagacatcatctttttactgttcttatcaccattattctattacagtttctaccctaaggagattagtgaagttgggcagaaattagaagggagcaaaagaggaatacaaataattatatcatacctatgggttaaaatattgaaaatttttcagcttggaaaagagaaaactaaagagagacataataactgccttcaattgtatggaggtttgtcacaaggagggtacgtaattgaatttttatccctatggaaagataaatcatttaaaatgggtataaactgtagcaatggggagcttatttgtttgttttctatttccaaaaattatttgatctgacttacaaaatcaaatacaatataaagcagaataataaagaccaaatgatctaaaaggagtggtgaaatagatgttgccaaattcagaagtgtaactgcaatttttgtttgatcctgattttggcattgtgtttttaacagctgaaggggaagggtaggagcacatggagggttacatagatagatcccatagactacacaggaaaggagcacactgcccatctctgatagccctgtaccctcaagagtataactgagcatcgttgaagtttaataggtcctttggtgtttggttgtttggttgattggtatttaatgatcttttgttgttgttaaattctattctactccatgcgtagcaactaaattattagaagatgaaactgctagtaagatgccaactgaatagaactttctggtatttttatcatatccaacttattttctgcatttggccctactttgaaacatctaaaatgctagatatttttatactaagattctttgaattttggagtgcaacattcagaatgttattccttatttctgccaagagattatataatgacatataaaactgtgactttatgtggaaaataaatttgactcaatttaatattctgggcacaacttactatcaactacactaaaacttgttctgtctggcagatattctctggggtctaaattgatcatttaaaaggacactttcaaccactcaactcattaagtggctcagtgattctgtttgcaagtagtccattccaacacgtctatcagtatacaattctcaaaattagtcgttctccaagggtcattccttgttttctcatttaatatttaaggtgtaataataatggctttctttttttcttatcagatgttttatccatccttcataacatagatctctggtatcccaacttggtatttgtccaagtacttgaaataatgttaaagatcatttttgtgtgtgtgtgtgtgtgaaagatttgaccgataaggattcttttagctattttcatgaaaaagtggaatgactcagtttctcaatgtgaaaacatattggggtcataagtgtttctaatgaaatatcagtcttgctgcctgaatttctggttttaaaacaagagcattgaactgtgaaagattagacccggatttaaatctcaaacttcctccaaactgtctgacaatggtcatcactttattaacttgtgctatttaactttgctgaacctccatttttttcttttgttttaaaacaaaagttaagaacatgttatgttcagtgtttttacattgtgcttttgtcagaacacttgtattcaaaacataaaggcacatttaagcgggtataagtaaaatggtttatttattataagaattcaggccatgcatggtggctcacgcctgtaatcccagcactttgggaggccgaggcgggtggatcgtgaggtcaggaggttgagaccatcctagctaacacggtgaaaccccatctctgctaaaaataaaaataaaaaaattagctgggcgtggtggcaggcaactgtagtcccagctactcaggaggttgaggcaggagaatggcgtgaacctgggaggtggagcttgcagtgagcccagatcacgccactgcactccagcctgggcgacagagtgagactccatctcaaaggaaaaaaaaaaagaagaattcaactgaaaatcccataggccaaaacccaagtaagccataaagagattaaactggaatgagaagtccagaaacatatcaacacagcctacattctttctgaatgcttgcatggctactgttatttctgttttttttttctgctttggccgatatcctctgcttggtcatggccagacatagcttcctcatcctcaactctaggtcatgcagtgcatcttctcataacattctgactcactcactcttacttcatttaaacatcacccagaaagcaaattctattgtttcagttaagcctatagatgagtgccccgtgatcagcgtcctcataaaacctaccaagctgtggccatgggaaaggggtgaaagagtcacatggtataaacatagtttatactttcagtatgggttacatggaggacaatttcaaaaaaggagtgagggctgagttggcacctatcatgtgttattataaaaattattcccattttataaatgatagaattgaagctaagcaaggccaaggtactattttaatatcacagttttgttaaggactacaaccaaattttgaacctgactattcttattctaagtttaatgatcttatcacaaaacttacctggttgagaaaataaatctccatgtaagaaatgattttttcctacctgagcatggtggctcatgcctttaatcccagtgactcccgatgctgaggcagaaggatcacttgaggccaggagttcaagactagcctaggcaacatagcgagacctcatctgtacgaaacaattaaaaataaacccaaaagatatgacctgttctaacatttaaattaattgaattatggatgcatctttaatcaacaaaccaatacctgcatgaatcgactcagtcccttaggtaccaaaagcttgcttttttatatttgtaatggatgcttttcctagaccattctctaaaacagtgtttctcaaaatttgcccattcattaactatgagtgctacatcctgatcacctggacaagcagtttaaacacagattctaggtccaccttagatctctgaaacaaaatcttgaggtgggaccttggactatgtgttttcaaaaagatctccacatgatttttatgaatgcaatggtctatattttcaaagtgtagtctctactagataaacagaaagcaatatgccatattatttaagttctccacaaagacgccttcgcttaattaaaatttggtattacattcttggattttaaagaaaaaaatattatctgaatgaagctctaaacagttgttttatggctacatgttttagtttccttagtttgatgataacacttgtgttagaatgagatgggtgggaaggacagtgacatttgcccttcctatagcccgtgcactgagcaggtgacctaggataaggcattgatcatgaattcagttattcattcattcaaaaaaaaattactgagcacactgtggcaaacattttggtttcttcaatatgttgcatagaatgattatgcatacaattctagtggcgtggggaaagaaacattaagcacaagacgtttagttgatttcattattgcactaaagtatttgcctttttcaaggctgaagttgacttctctgccttcctgagccatagggaaaagggtcttgtcttgcttatgcagccagcaagatgtatcggtttaagaaaagtgctgagtaatgcctgcaaatatagcaggcagtcagcctcttctctgcagcttggttcatgtcctttacccatgctatttaacccctgatgtttttatatccctatttctaaactcaaaatgaacctcaaactgaataaggcatacaataaatgtatccatattttctttattcaattcagtagcatctaaatccctcttgtgaggaccctctgctttggtcattagatagttaaaaagatttaaaattgctatatgtctagtttctaaaaattgctaaatgattttctaaggtaaacacctttccgtgtatccagtgcttaacagaattaatgttatgtgagttttcaaaaaaaagaaaggaaaaaaaggaaatctcttttttgtctcagccgacagcaagcagtgctgctccggaaagaaatgattttatttctagcctctctgcagcatctccctaccgcattagtatgttgctgcaaagctatccattgtgtaactttccccatcagtcagtcagctgtgtagttctctttcctatgaaatcgttatgccacaggagacaaaaataaaaatacaatgcaggttagttcattatatcctgacatctgtttcatgcagattgtgatgcaataaaatattagtaatttaaacagaatgtaattgtggtttgaaatcacttgggtaaatcgtgttattttaaaacaaatcttaggaaaatgaaatttaaactttgcactatttgattaggattaacaaacaagaatatgaataaaccaatagatataaaatattagactttagtacttagaatatgttttaagagtttgcttctagtataagttttaattaacaattattcattagctgaaaatttttgaggacctattaagtacctatcactcttctgggtaccatgggaggtgaaaagggggtttaaagtttgtggcgaatgtatagtagttacattttatgagaaaatgtctcatacctagtgactggtaaagacaataaaggctgtagaccctggtgactagtgaagaacctatggcctggcttctgcttaaagagagctcaatgttgcaaaattcctatttgccctttcacttttgctagaagtgtattgcatcacttgtaagattaaagggaaaatatacttattttgtttcccaaaagtttataggtttctgactttcacaaatgcttaagagtactcttctctttgttgttatggctgtccacgtgtagcagtttcatctttagaagtttaaattcttttctctttcttgctggatacaatggctcatgcctgtcatcccagcaatttgggaggctgaggcaagaggatcacttgaggccaggagttcaagaccacctgggcaacatagagagatactgcctgtacgaaagtttaaaaatcagctgggcatgctggtatgcatcccagctactcaggagcctgaggtgtgagtatcactcggggccagaagtttgaggctacagtgagccatgatctcatcactgcactccagcctgtgtgacagggcaataccctatatcaaaaaaaaaaaaaaaaaaaaaaaaaaaaggaaagaaggaaggaaggaaggaagataatttctttggtttgtataggacctacggagttaaagcgaagtgataaaataaaggaggagtccttaatgtgcaaagatgcactactcatatatttgaggataatcttacatcaatacttttaataatgaaagaaatgtgaaaatatgcttgccactggttttaacatttaccatcatccatgtttgaaaaataaaacccaacactggttcctctcatgagtatttaattgttcaagaaaaatcctagaatgagtgagttttttctgtcagtaccaccttacactctctacccccattccatctttttatatagtctcttcctggcacattgataattcattccatcagaaaatccccactaaaagtaattcatccatatgtattcttccactgaaattaacaggtcccagccagccccatcccctacctagtcaaccaaaaaaagaactcttacgccctctctctgattgattaacccactttgacagtgagaaatgcattttacattgagaagtaagtagtctcattgatgcataaacgactcatgtactcccagagatacttgtcttttaaaaggaagcgcttgctaactgaactgaaaatgctaattaattgaatttcaggaagcaggaaaataatttttttagcagagaagactttcttcatatatagggctgctacatttggaaactaaacatacaaactgtccagttatatttgactttcagataaacaatgaataatgtttactagcagtgggccccaaatatagacaatgagaacacatggacacagggaagggaatatcacacactggggcctatcgaggggtgggggcctaggggagggataggagaaatactcaatgtagatgacaggttgatgggtgcagcaaaccaccaaggcacgtgtatacctatgtaacaaacctgcaagttctgcacgtgtatcccagaacttaaagtatagtaaatataaaacaaattgtatgaggtatgcttatgctttaagaaatcagttatctgaaattcaaatttaactgtgtggcatgtattttttctggcaaccctgttcagatatcttaaatcattatggtggttactcatatcctccatgtcataatgctgaccttgggtctccctacttccaatctgccagtccagcccctttggacatcactgtttcctttacgcaacactctacctagagaacaacaacagttgtcgcaacaaatcaagtactcttctaggccaacttggtaacattctaaaacaataatgccctacctagcccacgggctttgggactttactgggggtgaatgtagggtagcaggtgagatatatttgggaatttaaacaaaagaaaatataatttgagcataggctttagctgactgtaataatagtatttaaattatttataaggttttaacaaagcatataattgaacaatttcagctgccacctgctattgaggataacaaaaaggaaaattttctcagcacttaagaaatgttagaaaacatattgatatgaaagagtagaaatgaatccaaaaaagcatgcatttttttctttagcaatttttgttttaactatatctttttccagttcttcatcattgacacctgaaaataggcattggcaccacaaaaatggttagatggagagaatgcaggacatgcattgaactttatattccattgtcttacatgtttatcaacattcactttgatgtaccaaaactatctttaaatgagatgcacattttaatgtacatctagattagagaaaaatcactctggtctgataactttatgggattgtgaatacaagtgatgtgatgggtggttgaccaattattttaaaaatggtttagtgacaattcaagattgtgttgtagctcatacccttacacacacacacacacacacacacacacaaacacacagattgtgggattgacagagatgttcagttttcaaagtcaatgtttcagtgttcaacctccattttcttctactctgggacttaaaaaaatgaatctgttaaaatcaggaatacctaaatttccaagtaaagatccaagaagactaaagcctaaagtctatttcataaaggagcttctaaaatcaatttggttactttgcaggattctcaatattattattctctatttgccaacttcatagtctccacatctctaaccagtgccttgcacattgtctatattcaataaatattaaagaaatgaacaaaattgactgtatagaccatgtttcttagtccatttagtattgctataaaagaatatctgaggctgggtaatttttttttttaaaaagaggtttacttgacttatggttcttcagactgtacaagaagcatggcaccagaatcagcttctattgagggcctcaggtgacttcccctcatgacagaaggcaagggcagcctgtgtggggtagagaccacatggccagagaggaaacaagagacagggtggggtgccaggctttttttctgagatggagttttgctcttgttgcccaggctagagtgcaatggcatgctcttggctcaccgcaacctctccctcccagcttcccaggttcaagcgattctcctgcctcatcctcccaagtagctgggattataggcatgcaccaccatgcccagctaattttgtgtttttagtagagacagggtttctccacgttggttagcctggtctcaaactcccaacctcaggtgatccacccacctcagcctcccaaagtcctgggattacaggtgtgagccaccgtgccaggctgctctttttttttttttttcaaccagttctcatgagaactaacagagcaaggactcactcgttacagtgagaatgaggcaaaaaccattcatgagggatccacccatgacccaaacacttcccattaggccccacctccaacattaggaatcaaatttcaacatgagatttgatggcgacaaaccatatccaaagtatagcaccaagtatcatacacctagacagaaatagacttggctcaatgtcacatctgctgtgacagatgtgctggacatctggtagtgtccactctatacaaatctgtgataatcatcagctgctgagctctcaaacaagacgaaactgtataataatccctagagggtctctttaacccagaccagtataccacaaaaggaatatagtgagaatgactgaattcacatgacagactgaaagcaaacttaagtagacgtttatatgattatcacctgaagaatgatgaggttcataaatttggaaaggagagctttatttcttataaagggctgcagcctgcagagtgaccttctgacaggttgggaagcataacttccagccagaagccagaaacagacacttcaagggagggaaaaaggaaacagaaatttatgctaagcacggtggccaaatatatatgttcaataagttataggaggagtcatgaatatttatgagaggagaagcatgcacatcctcaactgagcttcatgcccctttgtgggatccatgtggaaaaaatggcgacattagcatgatccgagggtagagttttcggccctctgacattaaaaggtgaagcagaggacacaaaaatccttactgcacattctcagtggaatggctagaatcattccctagtgggtggtctcttatgaggcaccaatggagggaaagtgtcaggtggttggtccatgtcagtgggggagtcttttgaaagggctgatttctgtttagcccttagggaagaaagcctcatagtggttagtgagggaggggttataaggaggtatgtctgactccctgatccaccatggctgagaattcagtcttcaaggttactctgggatccttttggccaggagaaggtcaattcagtcagggggcttagaattttattttcagtttacatgataaattaagcataattttatatcataaatgataaataacatacagaaaatttaatatactgagtttctgaatcaggaaattatctctgcctttcatctttcaagacctattttctgtaagttctttcagctttctctataccaaaatggttcttctctgaacaataatccatagtaaccattgcagataatatgcctttgcttagacttgttctatgtctagattttatatatacagaatctgaaccaaatgaattctgcagctctgatacttctagtctcaatttttcataactcagggaggttccatttgtcttcaggttatttggaaaagtgaaagtgtcaaacacctgatactcattccccagggtagttaatttgtgctcaacactctcctctccccgttcctggatctgtttctctttcccctgcagtttttctctgcttcagccccgctgcccgtgggttctatgctgtatctctccatacagcaactatgcctggacagttttgctttgtgttgaggtggtttcataagaatgcttttgagcagaagagtatttttaaattgagtttatgctaaagagaaacccctctgagcagaattccacacagaattctgcaggatatttcctgcataacaccataaacatccttctggctgcagcagctcaagtatttctgttgaaccatttcacaccctgagctaactaacattcagagaatgaaaggtgaaagtcttaaaacataaattaaataactatgatttcttaataacactaatgactaaacattccacatagatcctaccaaaggagggtattgtatgtctcccaggaacagagtctaatcataccattgttgtgctggaacaccattgagtccaatagggatggcatcatggccaagagactaaagaagagacttgaagccagcaagggagacataggatttatctaggactcatatacaggggtggtccagtggcagtgggctggatgggagaaccactaatgtttgtaaaaagcatgcagtttatatagcattttcacttagcactctccacctagcaacattcatttaatccaaacaaagggcttcatccactgtatggcccacattctgaggaatggaccaggctttggaaatccttaataataaggagcaaatttccaggttggccacttccagccagatttcttacctcagacttccaaacacacattctttttagaccacagagtcattctcagggtacccttaaattattgctgtcaggtgcgtctgccatacaaccatgagtcctgaaaggatttgcacactgaaaacactgcattattttaccaaaatggttatcaaagtagatatttccaaagaaggaatgtaagcatccttcctcccaacccccaaccagcattttgtgttaaaatgctagtgttggcatctttttccttcaaagggaattgtaccaagcctgattttttctcccattattgccccacagcaataaaaagacaattgctgactcaggcactatttcctctggctgctacttatagctacataactcctggctttaccagcaccataggcaaaaatgacctgcattgtctgagagagcagcaagaagaatggcattcattctgtctccatctttgtgggtttacattaagttacttctagaccatggtttttgaatgaaatagaatggacttgaagtgtctacaaaggagaggcacagtccagagtgggaaggggtggggagaggacagcaacggaagtgagatggaggctgagcaaaaaaaaaaaaaaaaagaaaattgagaataaaggagagctgggagagaaaagatgaacatcgagaaaggggaagatgaatagactggccattaggctttctcttcgagtaatccactttgtgcaaaacctgccatatacaacttggatataggcagtgcctctctcattgctagctaaacagccctgggaggcccacactgggctttaagtgcctatcacctcacacttggtatctcaaaatctgcaaccacaatcacagtcacgaggattagacttcactctctacatcctcccaaatctctgtcgagtgaccactcacccacacaataagattacttattttctaacaggaaaactttactaaaagaggttaacttaaataatttacttacaatgacagtcattactatattcttgatataaactaatgacatttgttcaaaggaattcccttaaattattctgactatgatcgtctagaagagatcctaaatttcacatgatttattatgtgaactatccatggtcttactctttaatgacaatcttctctgtaaggaaaaaaatgaaattaatcatcctaatgatactgatgtgtttcatgagaaaactacaggcagagcctaataaactatggcagatctgaatctcaggcaaaaactatgtactgtatttctatgtattgtatgtagattgaatgaattttttgcctgagatctgaatctcaggcaaaaactatgtattgtatttctatgtattgtatgtagattgaatgaattttttacttcttagtttattattagggtgtcttgaaaaacacccctggagtatacaaatgtagatacagacatatgttagatatctgaccttattttaactgcagtttgcataaggcatgaaaaactaagggtagataatgaagttatttttaaatgtaaaagttttaaacatatggctgaaatatgttgttttagttaaccccaacctactaaaaattggcttattgagaaatcaaatgaaatatttttgtaattataattacaccaaatcaaattatttctagaaaagaaatttataaaacaatactttctaggtttgggatttctatgtaaaaaccaatttctttcttggctgctaaaaaatagttatgtgagatcacaacagtaagaataacattttacctagttatttttattattattggcagtaacattcttttgtaaagtaggtggcaagaacactacaaaacgaactgaaacacttcacaaattgtcgtgcccagatggtttatgtccccagaatagtaatagctctttacaaggagatggcaagcattttggcagaagtctttttggtttctcttcagtatggatattaaactactttatagacagaattgaagatatataatttttctccacatcagattatatattataaggtggtgaagatatataaatgttgcacaactatattgaactttggagttacagataaattatataaagtcatctgctcagttagtaactagaaataatccagtttagcaatacataaaacataacggaagtaaaacataaattgacggtggaggtatagttcagcaggattaaaacctgcatcatatgattctaaacttgaatattacaatcatctttttaaattttggagtctcccatttttcaaaattttgaagtctccatttttgccagaatgctttatttactgtatttgttttgggtctgtccaagtaatataaattccttggagagacctagatcaattagagtagataaatactattttaacacagctacttatattttcatatattttatttagctttaaaagaatatttcataactattaggacctggcagacactggtttgggagttagaaaataaaagttctgatttaagtcccaccattaataagctgcgtaacctgggacaaatgacatatttattttgaaagtaggaagcacataagttctaaacaaagaaattgcccatttttctttgattcagtttgttttctattaaatgagaggaatagactaatctctgttttttaaatctttgccatttattggttttgatttacttatctttgtctaattctcttaaaataacttactcaacctttcagaagtccagaggtcaaggtaaactaggtcttgtctgcctgaaagcctgtattatagactaacactgttccacatcaacaacaaaatactatcacattaagtactatatatattcttgtctgtgaataaaccataagagctgcactttatattcagttcactaatcaattggttagtaatttttttccctgtaaatcacttctagggtcagataaggcaaaaaaataatagcaattattctaatagatcataacaatagcattggaaactgggaggagaaaatgagagaattgtctcaccaggttgtagggagagacgggggagtgaggagggggaatttcttttaggctttaaaaacaacatagaatttaaggacattattgaaccaaaaaatctcaagacattaccttagttatggtatttcttgcataaatacacaatggcatcagagtgtgaagttgtggatgctgggtgtctctcggggatgggaaacactaaatttcattcatctttctcaggttctgaacaaagaaaaaatattcagaggactaagacagatatcacctttttatctcacagaagtttgtggagagtatagctttgtgtgaaagcccaaaagcccgttaaccaaacagatctaaccacagaggcccaagcagtcacacagagctgccctccaaaggaagaaactacataatgatttacttataaaaggccagtttcttcgggtacaaagcatttataaggttgcctttacagttaagacttctgtgtgaaatgccacagggaaaatgaaatacttcattgtgagctatcgtacatatttgtttgactggagaaaagcagatgcccaagggagtttaactgtaaggcttcatgggttaggattactttaaataaagctcctccatctgcactttgagggtcaccaaagccatatggtttaccactcccagtcacctcaagtataaacatcttttggctgtgataaatgcctcctcaagtccagaacgtaatacatgtgtcctcttctacgggaaacagagatatataatgcccagaagcaaaaacctcatctagtactagaacatttatgataagtaatgaattagtaaagtcttagaaatgacactgaatattatcagtattaactttctgatcttgtaaagtctcccgagaaggtaatctgttaattataatgctacagatttacctccctctttttctttcagttagatattaccagatgatattatatctttatttattatttgtatattctaacaaatttttccttataattcttttgtccacgttatttgatattggtcatgttattgctattactttactatttctttccactcaaaatttccacttcattttcttacaatcttttttttcccccatgatctctcacttccatttcatgttcttttggattatttttttcctgtcttctttaggattgcttatctaattgaattccacttttaatgtatttttcttgtcttccaaagaatcattcttcttccagttttctattgcccactactctacttatttccaatattttttaaaattccttcagatttgaagtttgctaacatgcactttgttactttttcttcctagctatggtttcttagttctcttcttaattttattttgtccaaatgcataaccataatcatcttgaaaaggcctctctctttgaagtcttctcttgttaggaaagtttcattgtcttacctcttttgaaaggagcaaggtctcctctatgtaaccaaatatttaaatgctggtttttatgaaaagagaaagctgtaagccaaaactgcagattctatgaggttactaccccgtatctagtgacatgtattttcataaattaggacaattaagtcttctgttataaatttttagaataaagaggtgaaatctcctttgtaagtgtaaatggaaaattcatgatttatttctgtattttaatagaataagtacagatttcaaatgtaatttagaacaatttttcatattactaaaaaatattaatattgaatgacatctgacagttttttaggtatttcataattaaccttatgagatttttaatgagttttattcttataattaatataacataatttatttttaggtagcatttgaatagaagatctacttagagttaaaaaaatactttttctagtatcagtatgatttcattataatatgggttatattgcttacatatgaatataattcctgtatctaactaatttataattatatataatacgtttgaactgatcatatttgtcagtgtagaattcatgtcttctactttcttggttgtttatttctccagtaaatgatagctagtgaccagggagattttctttagagagcttttctcaaatgcagtatttctggagaataggaaggcttttagtaactgtttctgtccattcacacaacttcatggacaataacatcaagtgtggcactctgaaagacggtgattgtcgtaatgatcacatatgttaaaatatacctatatatgatatctaaacatatacatattaaggaaagtgctaaaacagtaaaatgaaaaggatgaaaccgaattgatggaaaaagacatactagataagtgcaaataagtagaaattaaagtcattatatgctatgagacaaagttgtctttaaagggaagaaactacagtaagataaacaagggccatttaaggaagtacagggttcaattcactccaaagatctgatcataagtgttatctacacatcaaacaataccgcattggaattcatagaacaaaaatgacagaaaccacaataaataaaaacacattaaatagacattaatttgcctctcccaacttggaacagtcaagtagaaaaaaaatatgtacagacatagaaggtctacataagataagaaataagatcaatataattgaggcataatgaactctactgctttaaaataaagaataagtttctttttaatgttcttagaaaagtcattttaaaaacaacctctattaggataaaaataaatcacaataaattccaagaaacagaaatggtacgaactacattatcagagcatagggtaataaaactaaacataaatttaaaacttagaaggaaaatctatcatttgaaaaatttaaacactttcttttaaatagttcttgcatcagtgaggactttgaaaccaaaattcattttaagaattttctggttattcttgtttgcttatttttccacatgaagtctcaaatagatttctctagttatatgaattttgtgattatttttcaaaggctgcatttaatttattaatttagggaaaattaatatctttatgatatagcattcctatctaataataggatataactttccatccatttatatttacttttggtttattcaaagcttttaaaaaatgcttacattgtatttagaaacatttcttccattgtactttttctctttgaaatagactagttttacagtagttttagattcacagcaaaattaagccaaaaatatagagtttcccctttctctacacacgcagagcctcccccaactatcaatatctcgcaccagagtagcacatatgtattactgtaaaaataatctatttagaagagaaaaaatacaatggaagaaaattaatttaggaaaaaaattaattttttccctaaattaacaaattaaacgcagttcaaatatacctgtattgtatataggtatattttaacatatgtgctatattatatgtattagaatcaaagtcctctttgattcaagagctattttagagacagtgtttaggtattgtatatagttacaactgatgaacccaatttgatatatcataatcacccaaagtccatagtttacattataattcaatcttggtattgtacattctatgggtttgcacaaatgtataatgacatgtgtccactattacagtgtcatgcaaagtattttcactgccctggcaatcactgatctttttattgtctctgtagttttgtcttttccataatgtcatatagttggaatcatactgtatgtccattttagattgcttctttcaattagtaatatgtatttaaagtttctccatgtcttttcatggcttgatagctcatttcctttaatgctgagtaatattccattgtctggatgtaccacaggttatttatccattcacctactgaaggacatcttggttgcttccaggtttgggcaattatgaataaggctgctgtatacatccgtgtgcaggattttgtgtggacataagttttcaactcctttggttaaatcctaaggtatgcaattgctggatcatatggtaagagtatgtttggtttttaagaaactgctaaactgtcttccaagttgctgtaccattttgcttgcccaccagcaatgaatgtgagcttctattgctccatatcctcatcagcatttaatgttgttagtgttctggacttcgaccattctaatgagggtgtagtgctatcttattgttttaatttgcatttcactaatgacatatgatggatgtggggcatcttcttctcatatgcttatttgccatctgtgtgtcttctttggtgaagtgtctgttcaggtctttggcccattttttattcagattgttcattttcttattgttgagctttaataattttttgcatgtttggggtaacagtcctttatcagacatgtcttttgcaaatatttttcttagtatgtggcttgccttcctattctcttgatagtatctttcacagagcaaaatttttcattttaatgatatcagattgtcaattcttttttttcatggatcgtgcctttggtgttatatctaaaaagtcatcacctgagttctggttccaaaatggcagtgaagaagcaagctggcacaattcctcctcaccatcactcctagaaaaccaaaaacaaatatatagcactgagaatatcaccagaaatattccagaactcaaatatgaagatgagacagttccaagaaaaacagagaactaaaaagttatcaacaaatttagtcaactaggttttctccaatattatcttctagtagttttttagttctgcattttacatttagatttgtgatccattttgaattattttttgtgaaaggcataaggtctaggttcattttttttttttgcatttggatgtctagttattccagcaatacatgttgaaaagactatcttttcttcattttattaccttgcttctttattacagatcagttggctgtaattatgtgggccttcttccacactcactatcctattccattgatccacttatctattcttttgtcagtaccacactgtattgattactgcagctttacagtaagtcttaaagttgggttagtgtcagtcctccaactttgttcttcttcaacattgtgttggctcttttgggttttttgcctttccaaataaacttcaaaatcagtttatcaatatccacaaataactgctgggattttgacagaaattaaattaaacctttagttcaagttgggaagaactagtgtctttgatatattgaacactcctattcaaagacatggactgtctctgcatttatttagttgttctttgatctctacctcactccatatagaaaataaattccaaatagataaaatatttaaacttataacattaaaccaccaaaatactaaaaatacaagagggaaaggtgatacagatgcatgtgtgtgtatgtatatattttatatcctttttcctaccccttactgcccatctctctggatgcctttctaagcattatacagtatccagaagccaaagaagaattagataatcacctaacaattttaacatttttaattaaatcttttaaaatctgtagcacaaagcaccataaagtcaaaaaatatacatttttaaaagaccacacatcaaaaaatcaagtggctaatattttgtcttaaaagatgctatagattgaaaaaaatagaaggaaaaaatgggctgtttttaaataagcagttgcaggaaagaaaataaaaaataattcttaagtataagaaaaatatattccacttttctcatagtaagacaaatgcaaattaaaattataatatgctctcatctttgtccctgtcagattggaaatatcaaaaatatgataacatattatattggtgaggataaataggcactctgttacattgctgtcaggagaataagttcatacaaactccatagaaagtagttttgcagtaactatcataaataaacattctgtacaaagccaagtctacctttaaaaattaattttataaatatatccacatatatttgaaagtatgtgtgtaaaaatgtattacttgcaactttattcaatattagctaaagattggaaacaacccaaatactcacattcagggaattggttaaataaattatggcacataacatgcaatggaaacccatgtagccagtaataggtacgagtcaaatctataaaaattaagataaatgaactctaatatacattttaagtaaaaaatgctggtgtaaaacagtttgtataataagattccatttgaatatgtattttaaaatagaatatgtatacatacactcatgttctagcatatacctattgaatacctggaaatatatatgggagataagtagtagtagttgcctctaggaaggggaactgggcctggggaaacaaggtaaaaaggagatattatactgcattttcttttagatatttgcatttatatcatttacatatattttctatcaaaaagctatttaaaaataaaaactatatatatacatcagaatattttatttcccttttgagaaaagttactatatttgttatccctaaacttttatttgcaaaaaataaatgttagtattttcctgatgccttgtaggggaaccagagtttgtagacagaacattcagttttcattgatttacagacaacctattcttgcaggtactaaataactgcaggttaagttctaagaaccgctgaaaacttcacttagtagactaagggtacaaatttaaacttgagtcctcccttcttttgatcatccactctgaaaaatatggttataaacctcacaacaatgagaacctccttgaaatttgaggtctctgtccctcacaaaataattatgcctgcaattttcttattgtagttcatatacaccaaagctataattaactgtaccatagcttgctgaccttaatgagttaatgcagtctccaaataaaattcatggccaagtggcagcttaggctctggaatattttggctcagtaatatctgtgaaatctgagctcatattaaatatgccttaaaacaatacatttgggatttggcgtctggcatcagatcaggtctgactctgctgtatggggttgacggttttgcctcaggccttcataatatggttagcactataggactatagaatcagcaagaatttcattattgcttcagtgaagaaaaacacttagtttctattgagtttatggcaccctgacttctttctgatcatttttcttgaaacaggagactcctgctgtctcctgcacctacagctatcttcttccacccccacctccatctagcaatttctttacagcctagttttcctctccttcttactcacataaatgacatttgtccaaccgtggactgtctttatctttatgtgttcaagtaagccaaccatgcatggctagtgaagtgagcaggacatagttccagttgtctcaaatcttagaagaggttaaagcccagtcaagacagcaataactatttgattggtcattaaggttggtcactgcaaactatcctgactatcttggccaggaatcacttgtcagacctagagggttagattttatgtcctctcagagacaattccttttcccctcatccttgttaaatgagctatcctttgcctttatacgcacagtctccatcttctgggtgtgtcacagactgaataactcattagtgatatagtgttaattagcctaatgtactttgattaaggccatatttgaaccacttttaaaactcatggcacagttcctgtatcagtaaagcacaagaattaataaataagtgatgcttaactaaactcaacacagaaaccatttgtgttaaaattttttctttagaaatcacctttcaatttaaggagaaaacagactttaaatcctctagctcatgtttcatgacaagaatttatttatattaacatctcttagtccactctttaaaatatctgtattccttttattttaggaatttcatatgcagaataaatggtaattaaaatgtgcaggatgacaagatggagcaaacagtgcttgtaccaccaggacctgacagcttcaacttcttcaccagagaatctcttgcggctattgaaagacgcattgcagaagaaaaggcaaagaatcccaaaccagacaaaaaagatgacgacgaaaatggcccaaagccaaatagtgacttggaagctggaaagaaccttccatttatttatggagacattcctccagagatggtgtcagagcccctggaggacctggacccctactatatcaataagaaagtgagtgttttttatcaggcatattttgctgctaattgcctactgcattccttggactgttgtagcaccaacacatgccaaattagcacaaaatcttaagtatccttctgtttaagaaatgaaacacatttgtgggtggtttatgcatatacatgagaaatgaattgattatatcataacatatgcaactgtcccaaatgcaccatgacaaataaaaagaggccaggtgcggtggctcaagcctgtaatcccagcactttgaggggccgaggcaggcagatcacgaggttaagagattgagaccatcctggccaacatggtgaaaccccttctctgctaaaatacaaaaaatttgctgggcgtggtggcacacgcatgtagtcccagtcactcaggaggctgaggcaggggaatcccttgttgaaccctggaggccgaggttgcagtgagccgagatgacgccactacactccagcctggagacagagcaagactgtgtcaaaaaaaaaaaaaaaaaaaagaaagaagagagggtgggagggaaggaaggaaggaaggaaggaaggaaagaaagaggcagctaatttaacaaattgacagattctctccacagattcgatttactatacaatttttttgtactttctgtacttggaaatacttggaaatctcaaaatcatgagttataaagactatacaaaatattagtactaatttttcatgcaaacaaatttgatgccacaagggtttgcaatttcaatgtcatgttataaacaacaaatcaattaatttcctgaagaaattttgttacctgtaaaataacatttttacataaactagaattttacaagttatttaatattttggtattaatgtactaatattggcaagaattaacaaaaatgtcataacctgaagaacctataaacaatatttgaatgcgtgcttttttcatatccatgcttgaaatgaatcttatgttaagcatccaaccgttcattatgactgtcatttcaccaaaccaaaacaaaaagcataaaattttttttaagattattgcattgattttaaattaaatgtatgacgttaggtgattcgtaacattttcttctttatcacttgacacaatgcatttgtgaatcttagtggaaaggtagggcacaaagattgtaattaagtaaatttttttgttggacctggaccttaaaataccccctaaacttttagggactctgtttctggcccagttctgtgtgaagttagtccttgccaaatgcatgcaaaagtttggcatttgtaatatgtgaatttgaggcagttttataaatcacttccgattgacagctacagtttcggaggaaagagcgtcacaaaaggttgactagccttaggttcagatttttttttaacttttcaggatatagtcagtttaagtataacatattttcccttttttttttttctttttgagacgcagtctcgctctgtggcccagactggagtgcagtggtgccatctcggctcactgcaagccccacctctcgggttcacgccattctcctgcctcagtctccccagtagctgggactacaggcgcctgccaccacgcccggctaattttctgtaattttagtagagacggggtttcaccatgttagccaggatggactcgatctcctgacctcgtgatccaccggcctcggcctcccaaagtgctaggattacaggcgtgagccactgcgcccggccagtattttcctttttttttttttttaaacattatacaactttaatgttacaccatctacaaatctaaattacatttaaaattatcagttgacagtggtactaggatttactttaggaatcactacatatttcccctttttaaaaactgatctattcattttacagtagaggaagggatggatcattttccatgtatttcattttcctctaactttgcttttgaacaccatattgtcaaatcttagttgataagagattattaatctcataactgccatttaagttatgagaaaagttgcactttattatttaatcaactctttggaaattaatgtatctgaaaagccaaagcccaaactaaacaaactacaaggactttatttgtataagaaaagtaaaaatgcagataatccaattgcctaaacattttaattcgtttaaaatgacttcggtattgaagtgaagactgatgtgttccaggcagaaattcattcacagtatagagtatgctactgtatttatagccattggatacttttagtttattcagaggctgctcttgggatatgttagtgggctccatcctcaacaggccttttgctacccccagacaccgggtcctcacctctttggtagcaatcaccatctaaaaattattaaagccacccagcatctatcccttaatcaaacagggaagaaatttagtatctctgctcctatgctgggtccttatttgtaaaataataaatgaatgttttgagaaagaccaaactaatgcatgttgacaaaggtagacattcttcatctggaaaccagaacaaccaatcaatcttttttttctttaattaaagagctttcagactgataaaagtgattgtctcgaggggatgtccctaaacaaaatggtccataattggtttataatgaattatatatagcaaaggatataaagacagatgtaaaacaaaattgtgcaaacattttcaaaatactcaaaagaaacatcttaaaactgagggtgaaaggccaactctgatgaagtgacagccagattcttgtgtcgaggaaacccttataaactttgttaggctcacgtctaaacttgctcttaacatcttctgttcttatcttcaaatgcacatggtatgaggaaagaaaagcattttggcattttggagcaaatcacagtgtgcaagtagaacaacctgataagggaaatattgttatcaggaaagagttttccccaaggtaaggtatactcatggcttttacattgaactgacagcaagcaaatcttcaaatgtaagtcattcaacagcaatgcacacgaagctttttgcagggctgaaatccttaaaaactaacatggtgtgcttagaacacatgattgagttatgtgtttgaaatttccttcttctgcatttttatgtcttaagacttttataagttgcctgtacacttttttctgcctttaattttcttttctaattggattatttcagtattgaaaaatatccttaaatggaattaatttgctctgactaaagttggaatgcttagcaaaatctgaaccttttaaaaataatgaatatttctttcaatattcgatgactaattttactttagtgactgggttatctgagattggctgggtgatcttcattaaaacaatgcggtacacatacacagtactcttgacacagctaaatggtcaaacagtggaaatgtcaccagggaaaatgtttattaggacgagattttccactaatgctgatactgggggtgtggattagatttgcctgagtgaaatggaatatgcagttagactagtgatttccctaaagttggactctagtacaatagttaatgtgtcatttgcatcagtatacaaagactctggtttctttctcaactatatggaatagacaaaacaagccaattgtaaaaatatttgcagaatccattccttatgttatattttatgttaaagtgcagtagtgtacatgcaaggaatttatgtgaatagaaaaaacacaaccaccacactgaagctctctgtaatcttttacaaaggcagtgtcagtgagcttattaaaatgtcactgctgagagtatattttccaatggcatatgcccacatatttttatcctctttgtcccagtttaagatgtgcaaattatgtaatgttcaaatattctactaaaattgttaaagcagtttaatccatgttcagtaatattgtagtagtaactaaagaaatagtagcatctatgcatcatgaacattcctgatgcatagaagcattagaaggtttttagatcctcagatgttttaagccttcagagttccaggttaaaaactcaggggaaaaaaatgctatcataatatggtgacagtatttttcatatcatgttagctctgactcagtggtctttagaacatgattccagattggtttccctttgtttgtttatttactttagctgtgtagcaaggcacattttagcttgtgaacacacctttttttccctcacgggactttttttcacagtttcctttcatagcttgggctgattttgtaccatttggccctacattcaaattctttacccactctttattacagaaaatgtcttccttctcatgttcttgttaaaattacagacgaatcattattaccatggaagaaaatatcattttaacaatgtgacagtttactggaaatagaatgagacattttagatctgtagtaaaaatggatgtttgttaattatggagtgcagaaatgcattcatttttaaaaagtcctatggatcatttaaaagtaacttattctctcttgctatcaactgtcctgaaaagatgcacactccctttttcttgggctccatgagaagtcatatatgaatttgtgatttcaaattcatgtttggtctatcactgccaaaggagtttttagttgttaaattgggtggagagagtttgttttgtttttaattggaattatggagtttttcttgcaaaagtaattaacgggaaatcagaaaagaggatgattattaaaagcttataatttcacgaattgtcatatgggcagtccccagtgtgtagctgttctttgcttatcatcagctcctgtaaaactaaaagtcacccagactctcccttacatccaggaccctgtagttgacttcccaatctttctcttccctatgcactatttcttccttgtgcttctttttcctttgtcacctccctgtctctgtctcagaaacagtcccaagatggtgatgttgatcaataaaatgatgcttggcatttttagagtatctgccatatctataggagatccttcaactgaaagtttccaaaacccaacagaaacaacttttggggaaaaataaaggaaatgtgtctagtgaatctggttttaatatcaaaatacccagtacctcaaacaatgtcatgagggtactccttttccatggtcagctctgctgtcacccatgctggcttcatttgcaggctggctcttccacaagatggcagaaatagccaccagtggtgctaaactaacatcctacagccttgtaactactatcttctgctaaaacaagaaagaagaaagaaaaaaggcccaggtttccttcaaaagctctagcagttgtcctagaattgagtctaattaggtcacatggcctcctctgaaccactcactttggctagtggtatgtgatggtctccttggtatgggatggtctctgtcatatactaacctgtgttgccaaggggtgggtcagctccaccaagccacatgggaagacagtggggcagagatttactccaaaaggaagagaagaggaactgttagaaaaggtgtcctaagcaggcaaaaaaaaaaaaaaaagctatgtactctacatgctatatataatatcagtataattactgttcttttttaatccacagattctttcaagatagctcttgtctttatttaaaaataataaaactgagtttaaataacttgcctaagactagttttaaaactagaccagtgagtttccatggcccattgccatgttagtttggatttgcagtcatagcactaatcacagcagtggaaaaaaaaaaaagagaagtcagtgatggaatctaaataagcctatagctgtccttcctgtttcctactcatgccattttgcaagatgtgattctggtctcccaggccggcagtatttcccgaacccttactggtcatttcaacacttggccatttcctgcctttgtaaaaccttatttatttattattattattcatttttatcaacatcaccatcttgggactgtttctgagacagagacagggaggtgacaaaggaaaaactaaaatcttagacactcagtgatctagattagcttctaaaataattagaaattttttttcccactaaattccagtagggtgataagcacctattttcaaagaggatcaattcaggaaatttaaattgtgtgcctctataaggaaatggcatttaaattgggatctgaaggttaaaaaggaggagggagaggagaatcttccaaggaaagagaacagcatagaataagacatataatcaagctgacctgtaataggtaagaagagaataaggtgaggttggggaggtagacctggggctagataaaggggatctttcagtacatcttaaagagtttgaaggttatcttcaaagcaaaatgaattcccaacaagtatttaggtacagcagtaacatggtccactttacattgttaaaaatcactcaacttctctatggagaaatttaggagttttatacttgatctagcattttgacaatggagatggagaataatggatagagtcagaattggttttggaggtgtttaggtgtcagacagaaggaagctgatggcctcattttcttagataggaagtttgagaagaaagatactgaacctgaaaattccaatactaaaattggccatgctttggagaaacagaaatgttcttatatgtgtatatttaagagttagaatacagagacgcattttaaatattttaatccttatatttttattgacactttagaattcactaagggcattgacatgttttatttcactttatcctcaaaataatcctaagattggtattagtataatcttctccattgagatggagaaattgaggggaatagtttgctcatagttctcacacagcaagcttgtaatcaagctgggctcaaaagtgttctttccagtcaaagaaaaattaactgaccatataatagtgtagcagacccatggtatgtacattctgatgatatagggctgaaaaatgtttcagctccagattttattctttttccttgttgttataatttcttctctctgctggagaagaggttgagggctctgcagccttggctgacaaaagcctacctaggcactttctagggaggcaccttctgcagagttatctcctccactggccccaaccctccacccagctaaagcataggaacaagcaattttacagactcttctctaggtacaaattcttctcttttgtgtgagcatatggcaacaatgtttaaaaactgactgtgaggcatggagtccacagacatgttttgttaaactaacaaaattatttataattttgaattattttctggcatttataaatcagaaaatctttcacacactcataaaattatacatctaaggattcctcttgaaatatgagttcaggcatcactgacttatatgcctaaaactggcaggtacctattgtcacttagcctctgtgggcattttagtttgtaatctgtggcttaaggaccacatctgtcattaagtgaaggctatatattaaagcttggtggcccacgggtaggcaagtgacacagattgttgtttagtctggttatcactggttggaaggaaaaaattgaatgagactaggagatgaaaaaatattaaatatggcatactctcttcagagtcaggtagaaagcctgcaagaaaacttttcatcccccagtgagccagaaaaggaagaaaagcttggcttgaactcacaggctatgacaaatgagaagggaagtaggtgagaccatttaaagtccttggggaaaaaatattatataccatggaattgacccagcataagaaatgtccccagagatatgtattatactaatggaatctcctgaccatctatgctgctcttcacacccaaaactttgaagcatctctcctcttgcaacatggctctaattctcccccttcatgctggaacctctttccaagccagcagcttagagctatgaaaaacttaatagcacttctgggtaggagctagagccagataattttcctggtctgacttccaggctatttcaaggacagaactctgccattttcagagtaacactttcttacacatagtgataaagtttctcagtgccattcattcagaattgtacttcattaacagtttgatagacctgtgggtctagaaatatatgtgtatctgtgagaatatatgttcacatttccaaacaattgacttccaaatgcatttctggagcacagctcatgtacaagttggaaattgcctgtttcaaactttgaagctaaaggataaatttaaccaaccagtagtatattctacattttgtgtattgttcatcatcatagctcaccttcctgtgttatttcttgtttttgccagtagatttactttaagagtaggaaagtgcaataaatttggagtcagtcctgttatttggcaatgtttcaataatgtttagatgaaacacaaagttacagcttatgtttaaactatgtactttgaagaatatggcccctagattgaaataaatgttaaggtaatggctatatttaaatgagaaaaagaaactggaaaaccatggcttgttaaattcaatgtccatcctgggaaaaaattgtaaacaaatcactttctataagcccagaaacaagaaaagattactagataataacaaatatagcttcataaatagcaatttctaccaaatcattataacataattattttcttagtaactattcagttagatcaaggggcaagatttcaatccacatgtacaacataatcattgataatttggccaatggtttgtgattttttacaaccattaggtaacaaacatattaacagccctgctatgacagaatcatctggattacttatttaaagtataaaaactaggtatcaaaatctgaccagctaaattggaatcacattacctctatgagctaagtacatttattctgttttcaagtgagataactgagtcacagaaagttaagaaacctgttccggatcacactgctggaaggcagcaaagttagcttttgaatcagaaatctaatttcagagtttgcacccttaacataagataaaacaaatcatacttatattttcataatgttgcttcccagattattaaactgttaattaccagggttattaccaatttttctatataacttccttcttgctttttctttaaaaccagaaatgatttttacatggcaaggattgctgaagaaggtatgagcttgctgaacaatggatggtttaagtttaggtctgtgctgcccaatatagcagagattaattagccttgtgtagctattgagtatttgaaatatgtaagtgtaactccatatttcaaagatctaagacttagtataaaaatatacatatctcaaaaattttttatatcaatttcattcccaaatgataataattcagatatgttgggttaaagaaaatactgttaaaattaatttcacctgctttgtttttcttttaatgtggctctcaggaaatttaaaatcataactgtagctcagatttgtagcttacattatatttctgttggaaagtgctggtcaaggtaaccttcatcaattcttctatattctattaaaacaaatagttgagcatctcctagtttataaatgtcttggtgtcataacctatgtatctttgttttctctttagaatgtagcacagggcattgcaaatattgtactaaacagttaatacatgtattctgaattgccatcaattgaaattaacaattggacctaaactaggtccctgctcatacataattaccaacttattgtatatatgcccaataaacttggtacctgtatatgaagacataagtgacaagctagaacaaccctattgtatgaatgttacattcctgcaaataagtctgtaaagtgatttttcatatataaaatctagttgttacctcagttacaatgccatgcttcaacatttgttttgaagcagaaaattatccacgtgttaaaatgatgttattaatttcaaacagttctatttctttgtagaactgcaattaaagcatttgttttggcactaaaatgaactgaaggacaaatattaagaatagtcatagatgtgcctgaattgtactaaaccacaaagtcttgctgcttgtaagtggagccagtcatgtcaatgttcctatgagccttgggtggtaaaaaggggtttgttttgggctagatactactgtataaaccttttctcctccaccaccaactttgaactgtttcttttttattcaaatgacttaatgcaataaaagttatcctgataagttgtattaatgaaatctttgttgaactaattagatttaaaggatgctaagatacttcactataataagctaaccaatacattcttttaaaaagtttttaaaaagtcacccaatttaatagttcataaatcttgattagcttaaagctacacaatatttgtttatacagtttaaggtacagcaatatcttagctagtctttgaactcttccctcttggagccctttcttatacgtatagcggggaaggcaaatatttatatatttgaaaactctagagctacttaagtatttgttgtaagcaaacagtatttccttcagactcttccctcccttcaatatcatcaatgccacttttgggagaaagctgtatctgagagtccttggaaacaagtatccttttattaattcttaacacttataaactgaatgatcggcttcttggctatgtaagcaattacaaggaggtaagatacagacagttgaggaaataaacaaacataagatcaattacatataataagactgacattctaaatggaacttactctgacatgaaagtgctatcatatttaaaattatacatattgggttgctatacaagtctgggtactgttgctttattggaaggctgtttaaaatccatcaataccttaaagctacatctatgaagtttgtacataagcatgtatgtttcaatctgagaaaaatggagttctcacagaagaaatcctatgaaaaaatggtcctttaatgttggccttattgttagcttcctgggtagcaaggatgtcttgcacaacagtatgtcttatgaaaaattaaagcaaaacaaataccgttgttcagtaaaaaccttaccaattccatttataaattcaacttgcaagtattttccattgcacatataaatcttgttaccaaataatcttctacacattctttaaatttttaaaatgaataaagggaatacactgtaaaatcagtaaatgttttcatctaaggcttgaacaattgataattatgcccatttgtttttaacttttgtgtcaaagtattaattttctctatgtcaaaggacttttagatttagaatcacataccaacattgagagtgctttggtttcattaaactcatcttacttagtttcagccctttatacctccttttatcttattttttaaattgacacgtaataattgtacatatttatgaagcatatagtggttttaatacatatgatgtatagtgatcagatcagaataattagcatatccgtcatctcaaacatttatcattttttgtgttgggaacattcaatatcttccttccagctatttgaaactatataataaattatttttaactgtagtcatcctacagtggtgtggagagctagaatgtgttcctcctatctagttgtaattttgtgtcctttaacagatttctctctgtccccacctttccccgatttttgtctagaatcctctgctctttttacttctaggcagacttcctttttggaaccttttgtgtttggtttctgaatcttgcctcttttactttttagtactcagggcagaaatatttcccctcccttcttttagatctctagctttgccttttactttaccactccttgtctgtcttttctgttttggtcatttattacctctggtcatggtttgggctgaacagtggccaaggagccagcatcacaggtgttctcttctaagtatggtcctgtctccttaggatgagcctggttattttccttatccacggaggtacaaacagtaccatcttgattatcctcacactccagcccatgatttaggccatctatggattgtcatttctttctcattatctctttgtatcactactatttgtttcaaaattagcttttaatattatctgaaatcaagagattcttattttaaaatatatatattctgtgttatatttttactggttctcctacctgtgcccctaatatttctttttctttttttcagatggagttttgctctcatcacccaggctggagtgcagtggcgcgatcttggctcactgcaacctccgcctcctgggttcaagcgattctcctgttctcctgcctcagcctcccaagcaactgggattacagggacccaccaccaggcccagctaatttttttgtgtgtttttagtagagatggggtttcaccatgttggccaggctggtctcaaacttctgacctcaggtgatgtgcccactttggcctcccaaagtgctgggattacagacgtgagccactgcgactggccatctgagcccctaatatttctttcagattttactcagcatatctggggggaaaggaagtatatgcagtacatttgaaaggtggaccatatttattcctgagctagctcactttgggcctttctgtgtcgttacatgctcatattcccccacagcaaaggcatttgcattgcacaagtctgttcagatttacgatgatctcacaatgtcattcagggacagaatccctcaatgaatcagagaaaaagagggctaagtattgttggcataggggattgttcttctagtaagcacaggtgaaattctcccttccttcaaatgcgctttgtggtaaagctctatctgttttctggactcatactccttcaaagatgatagagggaatgcaggcagaggtgagacttggagcagtgcttctcaaactctgttgaggatgtgaggtctctattcttctataaaatgaagtaaaaattagtttctagaaaactgaaatgaaaaacaggcccacaacaacatccccctacttttttattattagaatagacagacacaacattaattggccaaattgccttacaaatttctaaacatggactctccatatctatacttaaccagtactggttgggggaccatactttcacagccagtttaggacacttctacccataccagatagaaagtgcttaattttcaaaaaccaactccattagcaggtaggggtgtgggagggtgggtttgctttacttgaatttagaaaatcctgcaagtataggggaaatctgttaatgagctaagactatacaaagattcttagtagaaaataaacataacatttatgcttgctgtatcataacatgatctgtgcagatatgcatcctaaggcttagaaaagaaagctttcgaagaatgggtgaatttaaaatcaagaagcttaaaatttccttccccaaatatgctcttcacagagataaaggctttctccctggctctgtctaaatataagtaagcaccttggaaaacattgagtcagagccctttttatacccttacatgttgcttctttaatgttttgtctgcccactgccaattgatctcaaataagtctaatttttggaaggaatttcattcacattcatggatcctggattcttcaggagcttggagcttctcctctgtgataagaaagcactgcataagataggaagtcaattgggattagaattcaatatgtcagctaactcagaaatcccagtacagaatttagcactcctggtcaacgggtactctccagggatcttggcctccagaaatcaattattggattacatccatctaacccaagagtgggtttgatggtggctgtttttattttctattacgaagagagtgataatgtttattttatgaggatagatggtaaaaacaatctttatttcaaaaatagtatcagttctgttgaatgattttgcgttccacaggattaattttcaagatatttgaattgggaaagctgaaatgactataaaaagcatgtagtgccttaagttttttggaatgaaatgaaatgtgcacccatttaagactttcatttaactcagacacttaaaatatcgagaaaataaatgacttctcttggctaattgtcttatgtacaataaaattgagggtaagtattaggtaaaaggtaagtggtatcattggatttcctctctaattaagaagagatccagtgacagtttgttttcatggggcactttagaaattgtgattgtgctggtttctcatttaactttacaataatttattatgacaagtaacagaaagtagataacagagtttaagtggtttatactttcatacttctatgttgtgttcctgtcttacagacttttatagtattgaataaagggaaggccatcttccggttcagtgccacctctgccctgtacattttaactcccttcaatcctcttaggaaaatagctattaagattttggtacattcatatcctttttcaagtgattaatattaactatttgtacatgatctgtaagcactttatagctaaatatcaaattaagttgggaaatgtccatattatatagttttcatcactctcattttgcatctttgtcatattagcctcattcttaaagttcattaatcacatagacattactgaaacatgtactctttaacattttatatattatatataaacatgtatttatgtacttttgttatctttctaaaaacacttatatgtattgaatcctacaaatctaaagtaagataaccatgtttacctctaccatctttatacaaagtttgtgatggtgtagaattttcagtgagcaatgatatgataacagaaaaatctacttccagactcaagtttatccatagttatgatataaaacacttcagtgtatcttagaataaacctttaagtatattttctgtcagagaatgtcaggagtctttggccagtacttagttaattcttgaaatttcctgatgatttttgtgaaggaagtactggaatattggctgaatttcaaattcaagtcatgagttatgatgctgccatgcaaaactgtttgcttagtaaagactccggaagttaatgagagaagttggcatatatttccaaatggtcttaagggatttggtttcatgattcccattggtgttaaaaaggactccacgactaaattcatttgttaatcaatttaggaaaacaattcacttctgattgtatcttgtttgatagctttgacaataatagatattaaaataccattataattatcataagctcatacttttttttaaaggattctaaaatttgaacggccattctaattttaatatgacagcaagaaatttctttagtggtataaatatgctattactaaaattaaaagatttgaaacataaaaatcatatgatagggtttttatacttagggaagtttccttgtcccagtttgtattcctagcgccatcatgtggtcatttgatgaaaactttctttttcaattatggaaaatttttgttttggcagtaaactatagtggaaagaatattggatcttaatgtaggaaacttatattctagtcctggaccctttatctgattcctctagagcttagtaaggccttatttccctgatttttaaaatatagtgataaactaatttttagggtccctttcagttttataattctgttatgtctgttcatttgttttagctaccacttagagtatatttacttacgtaccgtgactgttccaagcatttccaatacattaactcatttaatctgcatgtagaaatggttacattacaaaatgagtacattattagctccattgtagagaagtaggaagagatacaaggcaatggcacagagaggttgagtctcttggccaacatcactttgctatgttgaattttaattacattcccatcatgttaagtgtcaaccttcaagaatattcacttttaatgttatcgaatcctgctgcacttgtataaactcagtcaaaatagagcaacccaacaattagcatcgtgctgtactttgactacaaattgttttcatgcgttgaactgtgttggataagatgtgtttaagaaattacaatattgcagataagtggccttcctcaacaatttactagtttattcacctgaatgagctcaagtcaatactttctgctgaaaaactgtgcaactccaggtagcacaaaaatagtcttcccactgaagttctctgggaggtgaatatattgatttaataatagttgtaaaaacaacaccttatactgatataatgctttcatatacattacctcatttaatctatacaaatactcagtgaaggtgatattattacccacattttacacatgaagaaattgaaatgtaaggagattagaagacttgcccacaatgcatttatccctgaattttggctaagctgcagtttgggcttttcaatgttagctttttgtaatataacacttggattttgattttcttttgtgtgttccttaacaataacctacattattcagcatgctaattatgtgcactattttgacaaactgtgtgtttatgacaatgagtaaccctcctgattggacaaagaatgtagagtaagttcaacttatatttttaataacatatatacatttgggattttgaaactgtgtcttaatgtagtcttaaaataaaactgaagagcattttattaaagtcattcctagacaaaattacgcagcaagaggacaatgctcattggccctcaggcctgctggcgttatactgattatcactctcttttgcctgtacctggtgttcacctgaagaccaccactcttggacatttacccctttttctttaaaatctacagtagccaaaccaaatgtggtcaaagcaattgcagaaaagagccaatatggtagacattggttccagttaaattccagataaatacattgaatcaagaatcaaaaagggttagttacaaatgccatgtgaagacaacattgaacctcgcaaagttttcatttccatcaggcacattcacttcatgtcaggccaatatagttccaagcgtggtcaccaacaaattgaaaaataatccaaagtttcagggttgtagaacttgaaaattcagtagactgaagactaaactaacagaagtggaactttttgtagagctttcagaaaccagaagcattactaactatattgatgatctctctgttctggggataccttcgaagcacagaggccagatttgtgataactgaagttgggctactctaaccatagctgctcacctgcccacacttcctatttgtaatgcctgttactatacagctgctgacccatagcaactacagaagtattaggttggtgtaaacataattgtggtttttgccattaccttcaattgaagctaatgtttgcaccaacctatttccaagagtgtccaccaacatttgcaccaacctatttccaagagtgtctcctttcctagacacttccaggctgaaaccaataagaaacatgacatcttgaagaaaataataaattttatatacaaaaaaattctaaagtattgaagctaatatgggaggccccacaggttttcagtcgccctagttttcttgtgattttatgccactctcacaccccaatttctgagtagaagatttaaactttggaaacacctaagtttctgaattatcagaagaattttattctaaattaaatttttacttcttataaccctagaagtatttactttgattttaaagaagtccttgttccaagcaagttaaattatcttgtagaaaaaaaaaaacacgtgctaatttgtcagtttcctctcattcttagaacactattaaacttgagatttttttccctaaaagaaaatcatttcatgcttctgcaatattattgattggctattccatcatgtaaacatactaaatatttctcaggagactatatttacataaataccaagacatttcagacaatggctaaatagatttcatataccttgtatttctcacactactcttaagacactttacgaaacaactctttgtgttaggaagctgaatttaaatttagggctacgtttcatttgtatgaaattaaaatccatctgcttagttttcttttttagtatttatctattccactgatggagtgataagaaattggtatgctatgaaaaaacactgttactttatcaaattttttggatgcttgttttcagatacaccttcacaggaatatatacttttgaatcacttataaaaattattgcaaggggattctgtttagaagattttactttccttcgggatccatggaactggctcgatttcactgtcattacatttgcgtaagtgccttttttgaaactttaagagagaacatagtttggttttccatcagtgcttatgcttttaagaataggtttgctttacctgtagaatattttggtgtgatttatacattcaaactctggatttcaatttagcacaacaaaggtctaagtggaatttcactatagcatgaaggctttgcagtagttgaaaactacaattttcacacgtagcatcagtcagaaaatttcacatatgccactcttccacacacacatgcacaaacacacacacatatgaacacacacaatctaatgtcaaaacacaagcatatttgtaatacatctgtgaaaataactttaaaaattacagccacaaaacagttctattggctacacaaatttgcaatgaccttgctgatattacacacagcttcttttattggtttggtaaaataattgaaagtaagtatctactagcatcaaaacgatctgacaaatcaaactaagagaagaaagaagatacttgaaatattatgtttttaatatattaaattactgatttctaagacctgtagaggaatacattctctctgcctctttctgtttctctcctgaccccttttctttcttcttttcttctttccttttatggctctcttcatctctttagtctgaacataaacttccttatccttatctcctattcatatccactcagtaatatgcacagtttatgcaccccaatgcttttttttctagtggcaaacttcctcaggaagagacccagaaaaataatagaaaaccattccagactaagtgaaagtagagtgtagaaggaggcagcccaacaacacttaccctcatccttccttcaggagctgattttttggcctctaacagttgtgatgtagtggataggagcaggagaggtggcactgatacactcaggactccgaatgtctggtcttaatttcctatctgatcaggatgagccaattctttgctaggctctctggtcacagctcaaaagaaggcctacaggacttctatttcttaagaacaaaatggcagtaacaataggcgcagcattggaccctcatcaagtattgttcttaatttgcttaacttttatttaaaagttacactaggaaaatcttatctcatatataaaagcagtaaggcaaactgccttttcatgcctaaatttctgttgggtgatttcatatatttttttctaactaaaaccctaaaccatagccaagatttactttctttttacaagttacacatgatgttggttgcattgctttccagagtgacaacaagggtgtctactttatgaagtttacatttagctaatggccttaaattatgtgaacaaagattaatgatggaatctaattcctgatttaccacttataatatttatcatttaaaatcaccaagaaggtctttataaaagacccatgcaagtaatataaatcctgctaaaatcttgaataattctgatttaattctacaggtttgtaacagaatttgtaaacctaggcaatttttcagctcttcgcactttcagagtcttgagagctttgaaaactatttcggtaattccaggtaagaagtgattagagtaaaggataggctctttgtacctacagctttttctttgtgtcctgtttttgtgtttgtgtgtgaactccctattacaggtacgtcacagagtttgtggacctgggcaatgtctcggcattgagaacattcagagttctccgagcattgaagacgatttcagtcattccaggtgagagcaaggttagataatgagacggacccatcatgtgattcagcatccttctctgcttgacattcagttttacagaaaatcaggaatcataagactaggtgtttcaaagaaaatgattcttattgtttagacataagctttatcagccttggagtttaacaatgtcttatgtgagatactgtaatttgaggtcaaggtgacctgcacttacaaatatcatgggtttttttttttttcaccatgttagttttatcgagaatttgctcccttttaaatgtaccaatctgtaatttcttattcattagagtcattaatgactatgtgtatacaatgcaaatcttctaagttttttgctaacaaaagagaagggaataagacaataaagaatagtgtgagtctgcatggaggatttattatcttaaggcacaattatactgcagaccattttcggttgtacagcaaaatctttcctacttataaataggaattttaaaatatttattcaggtattttgttataaaactgacctctgaccctaatgaaaatatgagacaagtttcagtagcaaaaaggaaaaagacaaggccctttccttaatataggctccatgaagaacatgaaccttccattactctatctcgacataatgggatatgaatcttcatttcattgtttaaatcattacatttctggagtttatcttcataagttcttccccttcaatgaagtgttgcttctccactagcgttgcaaacattcttggatttatgaatgaaatctatgttttcaagaaaattgtgtgaagcttttattatttcacacgtgttaagtcttcatagtaatagacttctttctttcaggcctgaaaaccattgtgggagccctgatccagtctgtgaagaagctctcagatgtaatgatcctgactgtgttctgtctgagcgtatttgctctaattgggctgcagctgttcatgggcaacctgaggaataaatgtatacaatggcctcccaccaatgcttccttggaggaacatagtatagaaaagaatataactgtgaattataatggtacacttataaatgaaactgtctttgagtttgactggaagtcatatattcaagattcaagtaagaattattgttatgtacatttccttaaaaagtagaattggattgtttgtaacacaaaggataaatacttgaggggctggatatcccattttacatgatgtgattagcacacattgcaggcctgtatcaaaacatctcatgtaccccatatatagaaacacctaccatgtacccacaaaaattaaaatttaaaatttaaaactaatgaaataaataaaacaccctctaaaaaagaattactatcatgtatatttggtcttttcattgctaatgtgccttccatctaggaatgaaaaatatttttaaatgattacataacaagacgtgatcttttagaaaattacctaatcaaactatttaacaatatttaaaattatattttatttggtatttctaccccctggattttaaataatagattatttatatttctggaactaaaagaagaattttgttaatcaccttatcaatggcttccacatacatttaacaactttgttcataactactctagttttgactcctaaatgccactttctattttatcatcatttggaaggctgaagaattcagctagaaagaattttcctaggatccttggttgctatacattcttcctgtggtgattgatataatgaaaggagataaaatatgtgaatgccctttgcgaattctacaatactatgcaaatgtaactcattatacattaattacacaatgtctttaatgtatcagaaactgtatagtacactggggaaaataatggaggtaatgcatggcttcagagtgttatagtttaatagattattttattaccttaaggaaataaagtgatacctaaaagtaaatcttattcttatatttaaaaagtagtattacattaaaaagaagaaaattacagttaataaagcttcaggagacagtaatagaaaatgttagttgggtgcagtggctcacacctataatcctagcactttgggaggctgaggctggaggatcacttgaggccaggagtttgagaccagtgtgggcaacatagtgagaccccttctctacaaaaaaaaaatatatacacacatatatatatatatatatatatatactttttttaatgagttgggtgtggtggcatcttcccatctcagcctccttagtagctggtgatacgaatgccagtaggtccacttcaatcaccagctccctgaaatctgttgtttgtttgttttatctaagttcaaaatactgactatgagggttatttagggtttattctctctagggaaaatttattggactgcatttttccaatactcgaagtaaaataatatctgaggtcattttcagctctaaacatctatggttcttattttaaaccaaatgcaaatataaataaaatggttcatttctgaaaataatagaaaattaaatttattaaaattaaaggtaactattataactgaaaaattatattttcctcaggttaaggctgatcattaaggttagatcattgaatttaatgggcaaaacttcatttataaatatgagatatcaaaaattcatccagaaactattatagtcctttctctttgtttaattttaattgaaagttaggaaatactatgtcaactttcctgtctaggttcaaggaagtcagaatattagaaataaaaatgatatttttccttatgtgcttctgtgtcctgtacagaactatgtatggaataaattcctcagaaaacttagcatctagaaaatatatcttcttacaaaatccaacattgattgtttaattccatgtttttctctttttgtcttttatttacttattcattcaataaactttacagaaaggctacccggtctcaaacaaacactaatatagaaataattaatgattgggactttctaggaggaatatttgtaaataaataattacaatatagtagtgtcatcctaagaataattcaagtatatacaggttactggtagcacagtgaggggagttcttaattatgtcaatatttagtagaaagaaagttgaaaataccaaggaaaaagttatagaagaactggacattgcaagaagccaaggagtttgcaagaaacaggaatctttgagataatgtagtgttcacctatagtcattattttagctttagtatttaagactttttaaaattgctttcatttcagttgttgatatgatttatgcatattttactataagttgcctatattctgcaaaatgtgagaatatgttatacttaattattaaaatgtatagtgttaaaaatataggcgagaggaccagaaaatatactattcttttattttgaaatatgtggctgttttgcaaaaacaaagaaattcctgtcattcctgtctatttttactgaatgaatttttttcgtgagaatggtgaacaaatgctaatacctttttgaaagattatgttagtacctgtcattatcactaactttacattcataatgaagaaatactttgcactgtaaagtgtctaaagtatctttgcactgtatctaatctaatgtcatttcttcataatgaagaaatactttgcactgtaaagtatctaatagaatatctagaatttagagggagaagagacaaatacttgtgcctttgaatgaataatatatttaaaattactcaataaacttaaaagtagaacctgaccttcctgttctcatttgagtgtttttaacaatgcaaatgttcatcatacgactttcttttttcaaacaggatatcattatttcctggagggttttttagatgcactactatgtggaaatagctctgatgcagggtaagtcaataatttgtgtgtatctgtgtatatttgtatgtacacatacatatgtgtatcttttctctctttttatagaaactgaagataacagccaactaattttattgacattaatttgctatatgtaaggtttttgataaagtatgagattatgtaaatattaggtaagatttatgttttctaaaacaagaagtaatacttttagacacatgcacacgtatgttcattgcagcactattcacaatagcaaagacatggaatcaacctaaatgtccatcaatgataggctggataaagaaaatgtggtacatatacatcatgggatactatgcagccataaaaaataactagatcatgtcctttgcagggatatggatagaactggaggctattatccttagcaaactattgcaggaacagaaaactaagtaccgcatgtcctcacttataagtgggagctaaatgatgagaacacatggacacatatagggaacaacacacactagggcctaccagagtgtgcaaggtgggaagagggagaggatcaggaaaaataactagtgggtactaggcttaatacctcagtgatgaaataatctacacaacaaaccctcatgacacacatttacctatataacaaacctgtacatgtacgtacccctgaacttcaaataaaagtataaaaaaagagaaaaatatctattaaacacctactgtatataacagcaaacatacaaaaagaggagtaatcctgttcctacttttttaggagtatataataataacaatgttgttcttttagataagtatattgagctataaactctggagactccatagtagtaaaaatgaaatggaggtgttgaaaatgcaaattatcaacaaaaattattttgtaaaatattattagaaatgctgcaccatattttaatgatgacaccaagtagctaataagactatatgcagtcaaaagttgggaaatagattagttacttatttgtcaaacttttattttgaaataccaaatctttctgactaggcaatatcatagcatagtatcagagtaaaaaggcagcagaacgacttgtaatactttcttttaccccacttgcagccaatgtccagagggatatatgtgtgtgaaagctggtagaaatcccaattatggctacacaagctttgataccttcagttgggcttttttgtccttgtttcgactaatgactcaggacttctgggaaaatctttatcaactggtgagaactaaagagccacactctccatttaagtaaaagtatacaagaaaaccaattgagttatgaaattaaaaccggatgataatatagtagaaagagcagaacttgaaacgagacttgagttcctctatcctattgattataacacatactgagcagagtgatgccaaggattgcaattctctcccatttcttcttggctcaaaacctgttccctacccttctcagacacagattgtcctaaacagaaggcacattctcccttcagccctatctaccccctcaaatattatccctcaggtgggaagtaatgaaaatgaaaaagcagatatagaaagataactttggtctgttccttgactttcctaaaggctcaggtcgttaatggaggactccatggcattttattgactgtttcattcttggtgaatacaaaaattctgccatagaaatcatagtgtgaatgcataagctagaagagaaggaagaagctgaagttaaagaaacaataaaaaaaaagtacagggtgctatgaccaacataattactatttatatctgagttttgctagacacatgagtaaattgaaagttgaagccaccacttagtgaaataatattgagaaataattctgatatttttgtttgcagacattacgtgctgctgggaaaacgtacatgatattttttgtattggtcattttcttgggctcattctacctaataaatttgatcctggctgtggtggccatggcctacgaggaacagaatcaggccaccttggaagaagcagaacagaaagaggccgaatttcagcagatgattgaacagcttaaaaagcaacaggaggcagctcaggtaaagctgccctgtctcatggcactgaccttttatcgttctgattgttacgtaatattgaagagaaagtgagtacctaagagcaggtggttgtatgaggaattaataatgatttctatattttgatgatccacttacttttatttagtttaattattttatttgagatgtgtataataataaccacaatagaactctctcatgctattttaatcatcttaagattctcaaagtaaagggaaaatgttattttggggtattttcaagtgagagacttttattcaatgagtagactggcattaggaggaaatcatcttttcttagaacaagaaattattggttatcatatgaagagttgctcctctagttcttaaacatagtctccttttttctttaactaaaaatatgttggcttttaacgtggttgctttattacctttataaggaagtgatcaaataatataatgatctctgatacagcagcatccaatagacatatgaatgttaccaatcttacacagattgaccatctaaaaaggattacttgtttttatagtgaagtgttagtcctcacagtgtgataaatcataaaatgcctctcctatctctctcatctttctgtttagcatacaaacttattgtctttactaaagaagaaagttgtttcagaaatattaatttatatgcaagagtaattaatttatcgaggttaccctgggaacaatgataataataaaaaagtgtcaacttagtaccagtacaagctataatcaaaaagcattgttttattccatgcccatgtgccccttaagaaccagtactgatttgaagatggtggggacagtttttggagaactggatgtcttatcttgctcagcagaaactattgattctcccttccccattcttctggatattgtggatgggtgagaaagggattttataactgtccataaataggattaataatattttgagggtattggacaatccaatttcaataactagagaaaggacctcaggtccttatcccattcccctgccttaaactcatcagcttttcttccatactcgagtttctttttcggagaaaagaaaacaacactaattaaataccaatttttaaagtaaatcaaatcccaaaaagtaatgaatttattttcttgttgatacatgttggatatttttgaatacgtggtctgtggagcattaacagagacataataaatgttaccatggagcaaactaaattatctccaaaagccttcattaggtagaaagaaaaaaaaaatctcctcttatacttgcagagaatcttctctgtgagatgatcttcagtcagttcaatatattttttaaaagccatgcaaatacttcagccctttcaaagaaagatacagtctcttcaggtgctatgttaaaatcatttctcttcaatatagcaggcagcaacggcaactgcctcagaacattccagagagcccagtgcagcaggcaggctctcagacagctcatctgaagcctctaagttgagttccaagagtgctaaggaaagaagaaatcggaggaagaaaagaaaacagaaagagcagtctggtggggaagagaaagatgaggatgaattccaaaaatctgaatctgaggacagcatcaggaggaaaggttttcgcttctccattgaagggaaccgattgacatatgaaaagaggtactcctccccacaccaggtatggcactgctgagtttactgatgcatggttgaaaattaaaacatgggagagagggggagaattagaaaatggactcaggaatttttatcaactgaatcaaccactgttgtgttatatttaaacccatcccttcttcacatagttatgcaaaaactttactccacagatatgtaagtctacagctcggtgtagttaagataacaccaagttgacagtcaggagaactggactgtcattctgatactgtctgctgatatttgtctataactaatgacatttaatctttttgatcctcagcacttttaagaaaaaaaaaaatgtggaactccggatagtcgatcaggaatgtccttaggttattttatgattttatgttttttttccccctaacttctttataagtcaccatgcatgcttctgaaaatattagtcatggtacacttgatcaaaaggcacatgactctgaatttcagaagtatgaacttcaagttccaacaatacaatgccttataggtgaaaaagaaataataattaaagtgattttagaaaggtggactaaagtaaattgtgaggagtgttgaaagtttttgaaagacaagatccataaaaaccaaggtggccttgttaaggtcatggtgccagtgttcactgaatgtttaccacgtccattgccatattctaaggatgtttccctttgaacttgagaaatggtcgttcagggtgtgtgtgtatgtgtgtgtgtgtgtgtttcaatatgttaaggttgcaatctatctcctcattctttaatcccaagggctagaaactttcttttatcaaggtaatttaatttaatgtgaatgcacataaaatgagaatgataatcaaaaggaatgaaccatattctgttatgaatgctgaaatctccttctacataatcttgcaaaatgaaatcacattcaaatgtccatattaatatgactttatttgtttgctctttcaaacttctagtctttgttgagcatccgtggctccctattttcaccaaggcgaaatagcagaacaagccttttcagctttagagggcgagcaaaggatgtgggatctgagaacgacttcgcagatgatgagcacagcacctttgaggataacgagagccgtagagattccttgtttgtgccccgacgacacggagagagacgcaacagcaacctgagtcagaccagtaggtcatcccggatgctggcagtgtttccagcgaatgggaagatgcacagcactgtggattgcaatggtgtggtttccttggttggtggaccttcagttcctacatcgcctgttggacagcttctgccagaggtgataatagataagccagctactgatgacaatgtaaggaagttttaaatagttcaggcatggctggctcactattgctgcaccagccagtgtttctacagaacggcaaccttgagaatgattcctggttggtcacgctgtgaatgcacctgcatcttgtaatatctttaatagactaaccaactaaaacttaaaaccttagcagtctcctgcacaaacctgaatgcatttacttattaaaagtgctaaggattgattagacacaataattactgcctccagttggaggatttgctatatacccacactgaatttagatagcaggacattctccaaggctctggtatgaaagtaaatgaatgattctatgtttaagtgtcaagggtatgtaataaatctttccaatggattcttcaagatctctgctttagtttagattttcaaaaacactgcttaagtagatagcttcattgttaccttaatactcctagtactcatatttttcttactaaaaaatgctgatataggtcttctcaaaattaaatgaagagacacaaatacattattaatacagacagataaaataattgtctgtgtacaaaggtatgttatgtgtttgtggatgtatactcagatgaggaatttctaaacagatgctgcctcttcttagactgtttactcacttggaaatatatcagcatgaggaggcatgactaagtcagaattagatgacttattgctttggtaccctggatgaaactgttgtccatgcagcatgtgtgtgttctttttaacatagagtttgtgttgtcaatgaccatctacatcttgtgtccagaaaataagatggcagctaagtgtgttgcaatcatttatacttccttaaaattaaatagctaccttctcaaaaatcaagtgccttgatcacttgtaggacttttaagttactaggcttttactattagttcagaaataagggtaataactgttaaatatatctcttaagagttttatcaactatattaaaattattttgtattttataaaattatgaaatcaggaagttaacatcttggtttttgctgtatgactaaatggttaacagtttgaacattccaggctaatgatacaataagtcagaaatatctgccatcaccaattgaatatgaaagtgcatgatgcatgtgtttcatgaaattcactgtgtcaccatttggttgtttgcttgtcatattgctcaaattaattgtttaatgcattagcatttttttttacagggaacaaccactgaaactgaaatgagaaagagaaggtcaagttctttccacgtttccatggactttctagaagatccttcccaaaggcaacgagcaatgagtatagccagcattctaacaaatacagtagaaggttggtaacaaattctattttcgtttcaattattttcaccaaacttatattgtctcatttcaaacaaatatatttgtgagttgggaatagtgcattctaatgaaaagacagtctaattcaagagctgttatttcttatatctactcagatattctagaagccttaacaatttattttaaaatgagtgatattgggactaagaatgttttcctaactgtgtagcaactctttgaaaatgcagaacaaagcatttctgttaggggtttctaatctcattagtcatagtgaccctctttttgttccattagtttaagacttcagcagtacagcccttttcagcctcatcctaagtactctcaagattgtagaggaagagcaggtcattttcacaaaataaaaacaagctaaaaagatgattcttgggtcacatcaatccctattctagctctcatagtctgaggttgacaatgtagagtcattcacatagggttgcacacacaatatctacaccaccctaaaccatgttcccattgagacgtcacatgaaagaccactcatttaacttgaggccaagtgctgagccacaatgtcagtgcataagacaaaaaactacccattgttacctgggctctatgtgtgtgtctgatgaaatatccttgggaggtttagagtaaactgtaatttttttaacaagtacaaaaaaggttgtctctgtaacaaaaatgtgttgattactgaaaataagtttagtggatatgaaataaatgtgtgtgtataaagtataccttttggtggttcttttttttttttttcttaatctagaacttgaagaatccaggcagaaatgcccaccctgttggtataaattttccaacatattcttaatctgggactgttctccatattggttaaaagtgaaacatgttgtcaacctggttgtgatggacccatttgttgacctggccatcaccatctgtattgtcttaaatactcttttcatggccatggagcactatccaatgacggaccatttcaataatgtgcttacagtaggaaacttggtaagcatattggaaggtaaatgtgtttagtcttcaaattttctgcttgaaaaactgtttacatttaattgtgtatagcagtctttcaaccatccttcatgcttcctggcccctgcaaaatcgcaattatatttagctggctatactctacttttttgccaaaaataatcacccttaatgtgctcacaaaaactgagaaaggcataggcctacagcactacttgaaaagtcaacagcaatatttataatttttcaggatccagaagtagctcatagattaagaacatactttttgagatattattacaatttcccttactgtagaagattaaaataaaattgtggatctcttttatccttcttagtcatcatcactgcaagaaataatttaacaactcaaaaatgtctccagcttttcctcttccaggatctaagtagtacatgtattgtttatccatctattctatatcaaaaccatgaactattgaaaacttccagtacattttgtgtgttatatgattacaaaggtcatttttggtgaagattgccacttaatttttcctttttgtgcttcaaaaacataaattattaaagtgtttgccagtcaactcaacttaggaatactagcttttatttgctgaaggaaagagctatctaaagtaatttatgcatttcctgtaatatttggtttgaagtttaggatttttgatggtagaaaacaaaagcatcatgcataacaacctgcattttattagttatatttttgaagttctactatttatactacagaagtaaatttatttttttcatgtgcataacaaagaatgacaatagtaatgcacctagtataatagataagaatgtataactctgattgagggaaataggaataagcttatagataaataaaagtgctcatgggcaaaatgttgtcagtacttgtggtatataaagtctggacaaaaaggtagtttggtataatggaaagagcactgttaaaaatagatgacttgggctgggcgcagtggctcacgcctgtaatcccagcactttgggaggccgaggcggacggatcacgaggtcaggagatcgagaccatcctgcctaacatggtgaaaccccgtctctactaaaaatacaaaaaattaggcgggcgtggtggcgggcgcctgtagtcccagctactcgggaggctgaggcaggagaatggcgtgaacccaggagcttgcagtgagccaagatggcaccactacactccagcctgggcgacagactgacactccgtctcaaaaaaaaaaaaaaaaaaaaaaaaaaaaaaaagatgacttgtactcaaatcctagttttgccattttcttgctgtatgatatcaagcaagccatttcacccatctgaagacctcagtttccttatctgtaaagtaataattgtatattatctacttcgcgtttccacaaggataaaattaaataatgtatatgaaagtctttcatcaactacaaattgccatacaaatttaagttagtaatagaatcattgtgggaaaatagcataagcattatgttctaagagcaaatcttatgtcatgtatgttattatctggtggaattagattaattttgttttgatcttaggttttcactgggatctttacagcagaaatgtttctgaaaattattgccatggatccttactattatttccaagaaggctggaatatctttgacggttttattgtgacgcttagcctggtagaacttggactcgccaatgtggaaggattatctgttctccgttcatttcgattggtaaaaaaaaaaaaaaaaagcaccaaattcaaaaacctttctaacaatcagggttcttgcatagcaatgtcatagtttttttgccacacaaccattagcattgtaagtttttctgtaacatttgcattgtcaaaaacttttcctacatggaaaaaaattctcaattatttatgaaaacctttatttcaaagaacatagttacgtaaaagttaacgtattactcagttgctattttcactaagtattgcttctagagactactacagttaatatttctagacctaagaaaatatttttaatgaaaatgcatcaccttttaatagtaacttgaaaatatatatagccctttgtagcagtggatgaaaatcagatgtaagatgtgcatgcagaaactttttctaattctgtctctggaaaatatgcatcttgcgaaagtcaaaggagagaaagtctgtaggattactaagcatgcccattaattcacacatatatccttacaagtcaggaaaataattgtagaattataggaaatattccaagtttttcaatgcaggtggatgtaactacatgaatacccctaaggagcattaatgaataatgaacactttacttatatcagggctcagtatgatgttactttgcacactactggcaaattaatatctttcagcagagatatttcagattcagatgttaatattataatatccggccttagaaccacctttataaaacaaattaagaacagttggattccaagtactcattaaaaattcactataagaggcctggcttggtggctcatgcctgtaatcctagcactttgggagactgaggcagttggatcacttgaggtcaggagttcgtgaccagcctggccaacatggtgaaactccgtctctactatatatatatttaaccaggtgtggtggcacgtgcctgtagtcccagctacttgggaggctggggcaggaggatcactttaacctgggaggcagaggttgcagtgagccgagatcactcctctgcactcccgcctgggtgacaaaagaaaaaaaaaaagaaaaggaaagaaaaataattcaccatcagagaaaatgatctacatggttaaaatatggaaaatccagcttttcctagggagtccaaaaattagccatgagcctgagacggttagggcagatcagatattgtaagatattgaattctaatgaccatttctaggtaaagctcaatatatataatgcttttaagaatcatacaaatatatattaatctttcattttccagctgcgagttttcaagttggcaaaatcttggccaacgttaaatatgctaataaagatcatcggcaattccgtgggggctctgggaaatttaaccctcgtcttggccatcatcgtcttcatttttgccgtggtcggcatgcagctctttggtaaaagctacaaagattgtgtctgcaagatcgccagtgattgtcaactcccacgctggcacatgaatgacttcttccactccttcctgattgtgttccgcgtgctgtgtggggagtggatagagaccatgtgggactgtatggaggttgctggtcaagccatgtgccttactgtcttcatgatggtcatggtgattggaaacctagtggtatgtacccacttaagatatgcattttggaaatacaccagcatggcacatgtatacatatgtaactaacctgcacattgtgcacatgtaccctaaaacttaaagtataataaaaaaaaagagtataatttaatggtgactgttttgtcaaaaagaaaaacaaactatgattattggtttaaaagtccattaccttggatatattatcactttaacaacacagcaatatagcagtgcccctgcattttttataccaaattctattttgtcagtcactttatcacattttttatgtgaattacaatagagtatcatattgagatgagcctaaaaggatgtgctgggaccattttataaattcagagccaaggaagagagaagtctaaaaaaagtagagagaaagtgcaaaattaccatccagactaggactaaaaagtggtgtctttaagagttcataatgcttgtgccatttctttgccccattgcagcatatgatattcaatcactctatgtcatatggcagatgcactagccaacctgagttcatcagttctcgaatggctaatgatgcctgagattaccataatggagtaactgatcaatctttatctctcttaaatttggagtgttactgtttatagtatagtaatacataaaatattctaaaggtatgtacattcatgtgccagtctcatggaaatatgagagattattcataaacagctctacttagtcaacaatgaagtatgagaaatataatagattaagaaaacaagtgtcagcctatgttaagctcctaatcttcattctgagttgtaatggttatttttcaattgacatccaatttaatatctttgtttattgaaaaattatagaatgcatttaaatgcatacttggaaacattagtgatttttaagaattctcgtattgtacacatataaatctgttttcttctactcatacaattttagagttaacaaaaccttagattagctcattcaatttcactttacgaatgggagaacttgagagcaacagaaatcatgtctttgtccaaggatgtgctattgagccagtcacaaattcagatcacccatcttctaatcactatgctgtggtgtttccttctcatcaagttttagaacttagagttttttccacacttaaaagaaagaataagtgattgtaatctgctcttccctacattggtgtaaaattataatcatgtttttgttgtttttaaggtcctgaatctctttctggccttgcttctgagctcatttagtgcagacaaccttgcagccactgatgatgataatgaaatgaataatctccaaattgctgtggataggatgcacaaaggagtagcttatgtgaaaagaaaaatatatgaatttattcaacagtccttcattaggaaacaaaagattttagatgaaattaaaccacttgatgatctaaacaacaagaaagacagttgtatgtccaatcatacagcagaaattgggaaagatcttgactatcttaaagatgtaaatggaactacaagtggtataggaactggcagcagtgttgaaaaatacattattgatgaaagtgattacatgtcattcataaacaaccccagtcttactgtgactgtaccaattgctgtaggagaatctgactttgaaaatttaaacacggaagactttagtagtgaatcggatctggaagaaagcaaagaggtaagattctataggtgtgggaaggtatgaatacatatacatatatacatatacacacatacagatgatcctcagcttaatgatgttttgacttaagatttttttttttttttactttatgataatacaaaagcaatagccattcactagaaaccatgctctaaattttgaattttgatcttttcccaggctagcaatttgtggtacaatatactctcatgctgggcaggggcagcaagctgcagctcccagtcaaccacgtgattacaaggataaacaaccaatatttaccgtacagtgtactgtattcaggaaattacctgagttatccaacagtttattataaatcatgctttcctttagatgattttgcccaactgtaggctaatctacatttcctgagcatgtttaaagtagactagggtaagctataaagtttggtagttaggtgtattaagtaagttttgatgtacaatattttcaactcacaatgagtttatcaggaactaacctcattgtaaattgaggaacatttacaatacatgcattatattgcctgtatttattattatatattttccatatagatttttttgacatcatgtaaaattttatttcactgaggaacaaaagatttaactcggaaatgactgataaattaaagggtaatatagtatacgtgtgagttttctcatccttagtaatggatatttgttagatgaatgaatgaacgatctaacaaatagacaaacatcatcagatttaatccttacaataacctggaaagaaatgtgttattatgttaacttttcagataagaaaattagcttagggagcattaggtcacatagctagtgaggggagtgccgagagttgaacttagagcccttgtttacaagttctgtttattgttttttgcatcacattggctcatgtacattgaagagaacttaacaacaagaaatattcttgtagctccttcaactaaaattacactgccctatgcattagcctgtttggactgccataacactataccataactgggtggcttaaacaaccaaaatttattttctcatagtccagaaactggaaagtccaagatcaagataccagcagcatcaatgtctagtgagggctcactccttgggttgcagacagccacctccttgctgcgtcctcacatggcctgtcctcaatgcatgtacatggaaagagagagaactctggtgtctattcttcttcttataagtgcaccagccctatcagaataaagtccaacctgtatgacctcatttaacctttatcaccaccttacaggccctatccccaaatacagtcatattgaaggttagagcttcagcatatgaattttgtggagacaaaactttaatccatagcaacctactttgaaatattaagttaaaagtctaatggatatataagataattaacaaggagtctaggacaaagcacctttacttcccatttagtgtttggaaatctcaaaaacagcagggtgcccatcttttaggctgtatagtctaaatattttaaaggattgaatacattaaagacattttattagagatacaattctattagatatcatcatgtccattgcattttgatagttgtccatggaagataccacaggtatgtgacattcttttgatttagtgactatatcccttaaaagatgcttcaagtatttataatgagtctctgaataatctaggtcagcctccacttttaggagtagggggctaatattggagctggaagcagctgccctgaccctagcattagagctgcttagttaattcagatttgtttgatgagagaagggaaagaaatatagtgcagagatgtaatagtagatagatcctgtcacatttccatgtctatggaaatagattcaggacaaaaaaaaaatagactgtttttatcaaatattgtggacttttttacagaaattttaatttggatattatgtcagttcagactttgtttatttgagttttcctaaggaatcagatgaaggctaattttttgaaaaattgttgtctttataaaattgtttctgaacttctgttaaaataaatttagtaatagcctagtctagcttcctttgtcttgaatctttagacctacatttagcatttgatgaggactaatggctaccatatgctatctgttatattaaaaaggaaaaagactattttacaaagaccatccataagatatacattttctgtaaaaatttgacatgattcttactcttattgtttttttatattgatcaaggctaaaagaaacactaacctttttataaagcaactatccactgaagcactagaggtagttctgtttctttctactttgtggtattttatatcttggaacattgcattaattgaaattttaaatgttaaggtaatagaaactttagagataatttaatgtggtgcttctcaaattgtgagtcatagtttagttagacttgagaaatccctttaataggccagtttttttttttaattttaatgaaatagtgatccgcccacctcagcctcccaaagtgctgggattacaggtgtgagctactgcacccggccaatgaaatagaacagaataacgttagagcacatttcatgtcagagggagaagtcctggtaaatgaagcttttgccacctgtgctgaactttaatgttttgtttgtctaattagaatgcaaccacttggttcatgggcatagtgtctgaagtccaggagttcttaagcttcagaaagactgtttcagctttttgaaggtagtgataatgaagccttcctaaccctaggcagaacagattgctttcttttttggttcccagagatataaggcatagagtatagtaataaataatgcatcttcttgactcatgcgtcttacaaagtactatcatattttagggtagctacttatatgcatgcatgtctctccttttatgtggtgacctacttaaacatgtttggtacaaagtagacactcagatcaaacttttccagctcattcaactcttcttaataaaaccattctgaagggctctcataaatactttctctgattagagcatacttgagattgtcattttaattccaattattttggaccaattttttctctagattcaagattcacttgagttagattttgttgtttttgtttttgttagccatgttaccttgtcactttacaccacccttctaagaaagtaataatctctcatcttttccacatgaattgctgttgaaatgcagttgtatctcatccagacgaaattttgtaattaaatactgaaacttctatttatcccctaatgatacatttcaatttcttttattcatttttgaatctggctttctttttatattctttcacagattaccacattgtatcaaatttcctggcacttttacttcacaattcctttcttcttggtcagaaataaacccacacaatattgtttgccctcattgcttcatcagtcttatactgtattctgttcatcaaagtaaggcataaattatgagatattctgcttttagaggaattaagctgacagcagatgccccaatagatgaagagtctttgttctacattaggaatgctgcctgtagtatattaatctcatcattttggttctttttgttttagcctttattctttttgcctattttcatttatttatcagttatcatatgccagggagactagataaaggagtatactaataagctataaggaatgattttctctctctgtctctgtgtgtgtgtgtgtgtgtgtgtgtgtgtgtgtatgactttcaacctttaagtatgaattttcatattgagacatatgttactgatgcaaaaatgttacctttcatggcctctattagactagtttcatttgaacatgttattgtttccattcctgaattccagtaatgacttatctacaatgcatcattataatacctccaaaattatattaatgccttgtgttaaaattacaagtgcccactgtttattcttcatacactaaatatttgtttttagacatgtgaggccacagttataaagttggtaaatggttatactctggagccagatactgggttatataatttcattttagccgcttactgtgtgactttacctatctaataatcagtttcctcatctcaaaaatgtgcataatgttactgactgcatttctgggacagagtattaaatgagataataaatgtgaagtacttagcacagtgcctaattaatatagaatatctggtaaatcatcattattattgttaccagttgcattccacactgttccagtaagttttcttacaagaattactactggtaacatactccactatttgtctttttttgttgtttgccttttgccctctgtaataaattgttttataagtttatctggggatggtgatacattttatattttctgtttatttttaaattgttttgattacattcttgatatggtaagctccttaaaatatttaagaaaattacttttctaaggggcattccaatcaggaaatacaaacatgcagaaacctaaagacaggaagcaagaggatgtatttggagacaattagtagtttggaattccgggggcgtaaatcacaggagctgaggatgagatgtaggtaaagagtcttggtttccaggaagcaagaatagactggtatgaagagatgtggtgaactggaacctcatatttattttgcctcttacatctgtggtgaactcacacctaactatcctccattccagagcaaactaagggctgttttttctacaccttcctcctattggtctccaaaagaagaaagggaatagcatgagtcacatttcatcctctctttaagtcctaagagacttcaaccttcaaagtacattcaagtaatttgaggaattgttttttaaaacctcagaaaatttcttgactcatgccatagtaggaataatgataagctagaaacatgaatttttattgaacattaataataaagaatggtgacttggacagatctgagagagctttgaaggctgggcaaaactgccaagactatgcaaaagacaatataaaatcttatctccccaaatagaaagtaaatttattttggttttggatagtattttataacatttctatcctccgaagcacctaccacaatgctgggtgcataacagttgcccaataattttttgaggtttggttgatttatatggatactagattaagagcttataagccttaactctactatgagcaaatcgctaaatctatattaccccgtttcctaatttattaaatgagatatctgtcctatgatgcttatagaaatgttatgaagatctaaaagctatttggaagttaaaaatgttcaaaagtgtaaggtgatgtcaagaaaattactacttaattccatttaaataaaaattttaaataaattgagttattaaagttgtaccttttgggaacaaaaaaggcacattactaaagtaattataaaagaaatacatttgtttttgcgatgagtgagctctagaataatgagtaaaaatgtgaggtccttagtaattcaggttatgttaataacagctattgtttaaatgttcaccatggacaaagtactattggagggcatatattgtcccattcagtctttatgccgtaagacacactgttattccagccttaagatgagaaaaataaacatattttgaacaaaggaaaacttgccaaagttacacaaacagtaagtggttggtcctggcagtctgctccatagtctgctccagagtcacgctcttaaccgctacactaaatcttaatagtgtctgaaaatgtactttctttggcatagttctgaaataaataatactatttgttgaagaactaggtacaggtgtcaactatagatttggtgttaacttccaatcactgcacattgggcataggttaacagcatagtctggagtgtttcagagtcctcctgcttagtcctgaaaggtcctgttcctctggtactggcctgcactttaggctgatctgcttctgcatcccacactgacctgtggtggtctaattctacttagagagcacttagtctcccagagtgtgatagttgtgcttaaggagcatccccttctgagtttaaagctgctggaagagtcttccaaatcattaggaagaacttctatgatgtatcattagcttatgaggtctttgagaacaggaaaaacactaaccctgtcttcccttaagtccacagcacatacaacatatttattaaatgaatgaataaataggtgatgggcagttacttctagatttccattttttcccaattatatcactgatatatatgcattttaaatctacttgttgctttgctgatatttttccatctaatacttttatcttacgttatatgggttaaggaacatacgacaatctccaacctaagcacatagtctctaagagatggcaactactctctgcgaagagtactatccatggaacccaagagtactatctccactacttctaattgatcactagggactgccaaagctacctatctttgtccacctctctgcattcccaaggcctttacccagtgtcagacctccatcatcttccagttttactgctacagcctccaaacgggtctcctctcgctagtgtagtcttgacaaaacaataggtggtctttctaaaatgcaaatcccgtaaccgttctgtcctccctaacctttcaggagttgcccattgctcacagtatggagtctatactacttaacatgggtacaagtccctgcaaaactgaccaccgctgactccttcagtatcatggaatagtacacctcacacctccccacccagctatactaatctttcattcaatgtaattccctatgctttatcagttctcatcgttataccttttctctctgacaggaatactctcttactctatcctgcttcacctgactcatcctcacccaccacatccttcttgtcccaaatgtcactttattctaccctgtccttcctagattgagctaggtggtgttctcaatatactctatactactcctattagagtgtgtggcaattgcttctctgtatcctccatctgattgtgaaccgacgagggcagatactaagtctattatgttcactattttatttttcaagtcctagttttgatgcctggcacatttttggtagtgaaaaagttcaaatatcaataattacacctattacaaacaatacaatactgtgatggaaattgttaccatttatcaaataagcatacgccaaacgttattcatgtgttatcttgttcagtccttataacaaggatgtgaagtaagaaccattttacaaattagaaacctaaagcctaaggaggtcagaataattggttaaagtccataaaaccagtgattgtgattcaaactcaagtttctctgatcctctgaacagtagtactttcaactctttcattatatagatatacagtttatttgcccagtagacacttattaagtatgttataatttaacattaaaaatcaatgtcaaaataacattatagaagctctgtgctcaatttggcaaaatgaatttaacaatgaaaattactcatttgatttgcattttggtttctagcttggggattataaatgcaatttttcagaatttttttgttttctttccaatttttttgtattaccatgattttctattgactcaatatgtctgaaatatgttgcttttgttcccttatatttaattattgatacagaagtttatgtgtgacctatcgaaagcaaaattcagagtagtatattagagccgtattcctccacgttcacttttatactgggcaagtgtttgactaaagaatagaaacaactgacattctgtaaatatgaataatcactgaagattgtcctttcaacatacagcttgatgaagaaatcaactgggaataatgtgggaattaatgagttaatatttgagttttctagctgccctgtggaaatgttgagtctcattacagtgtcttttccattgatattatattttcctttgttgagagtctttaataggggtcattttaatttttaatatgcatagtgtacaatagaaatatttactagtgcacaatgtacacaatgaaatattaatactagcatttctgaggttgtagagaacctcattttagcatgacttaggagatatatgtgtcacaatagtaaagtacccagcattaaaatagtacctgtccagctttaaaaataagtactttggataatattctttatatgttaaatatatacatgtgtatgttaaatatatatattaaatatatatgtacacacatatatacatatacaaatatatagacatcgcatgaccacatattaagtatgcttttgacccttaaattccagatttatttatattacttattataatttgaactcctactaaactctataaataagctccttataacaaaccaagtttaaaaataaaaatataaacattgtaggatcaagtcagagaacaagaaaaaaagcatttaatcacttactctcagagaatattgaagaaagttttctttgccttgctttgttttgccttaccctcaaggtcaaaatcatttgctatagtaaagagcattgttaaaaaacaagaaatttgcataaatccactgcggaggaaaactgctcttaccatcagggttgtagttattaagtggttattgaatgccctggtgtacaaggcaaagtggatgcttcatatacaacgtggtctctgcattctgtcagctcacatattaagtcagcctgccaaaaatacatggatgatacaagtcagtgtataagtgtataatttagctattaagattcatgtgtgaaaatatacatattcttaaataaactagcctatttagaaattggtaaaatttaaggttgattcatggtttaataaacattaaaatagattttaagaaatattccgggaggccgaggcgggcgaatcacgaggtcaggagatcgagaccaccctggctaacacggtgaaaccccatctctactaaaaatacaaaaaattagccgggtgcagtggtgggcacctgtagtcccagctacttgggaggctgagacaggagaatgacatgaacctgggaggcggagcttgcagtgagccgagatcacgccactgcactccagcctgagcgacagagcaagactcagtctcaaaaaaaaaaaaaaaaaaaaaagaaagaaagaaagagaaatattccattaatctctaaaacagtgagaacaaattactttattagtgaatagcaaatttcaaaatgaaattaaatttcaatctaagaacaaaaaaggaaaaattaattggccttactgatttttaaatctgtaaaaatacttatttgagaatattttagttttgccgttattgaatttctttagaattttatatgaattcgtatttcatgtgctaacatagaggtggacttttcctacacatctgaagaggagcttttccttccctttttgagtatttaataataatagtgttattatcccaatcaaataaagaattggaccacttcatcttttatctcaaccgttataagaatatattgatgcaaatatactggttatgaaggttttaatacaacttttttagcctatggtaaaatattttaaaaaatagatgacaacatttagattttggataatattaatagcaaaagtaaatatgactgtatattatctcaaaaaacatatttacagaaaatttttattttcaagggttcatggaatatttctataaaagtaaccatatactggagttcaaagtaaagctcaataaatttaaaatagctggcatatagggttatccagtcacaatgtagttaagcctgaaattaataataaattgatagaaagaattccaaccatttgaaaattaagaaattgcctcctaaacaaatattgaataaaagcataataaaataccaaaatagctatttcaaaaataaaagcattttatgtcaaaatttgtggaagaagaccaagtagtcattaatatgacttaatattactaatatgattaagaagggatgaaagaacctaacaaatatctttgagaaattagaaaaggaacaaaaaaggaagaagatatacataaagagaaaaaatagtgaattatcaatgtagcagacaatattggtataacaggttatggttcttaggaaaaaaaaatcagtaagacacacaaactttggtgtatcaggcagaattctaagatgatctcaatgacacttgcccttgtataatccccactccatgagtgtggttggaacctgtggatatggtatcaccctttaattacattacatttatgacaaaaagggggatgatgctgggtgggcctgacctaatcaggcaaactctttaaatgcacagtgatttctcctgctatcacatagaggaagctgaaaagatatgctctgcctggcctagaaaaaagcaaacacccatattgtaaattgcatacagcagccatgtggccagagactatgggtggcctctagttgctgagagtggttctgggccatgagctggcaggaaagtaggaagcccagtcttataattatgagaaatgaactctgccaacgacagtaaacttggaagaagatcctgagccctggaaaagaactggatttccagccaacaccttgacatcatctcaatgagaccctgagcagagaatccaactaggcagtgccagacttccgacctacagaaactgtgagttggtgaatgtatgttaagatgctgggtgtatgctcatttgttacacagacacagaaaacaaatacatttggcaaataatccttgttaggaaaactcaaatgattttctatgtgtaaagagcaattgcaatcaaataagaacactaatactccaatagaaaaataggcaaaagaccaagcaccgtggctcactcctgtagtcacagcactttgggaggccaaggtgggtgaattgcttgagctccggagttcaagaccagcctgaggatcagatgctgtctctacaaaaaatacaaaaattagccgggtgtggtggcatgcacctgtagtcccagttacttgggaggctgaggcagaattgcttgaacctgggaggtagaggctgcagtgagctgtgacttcatcactgcactccagcctgggtgacagaatgagacctgtctcaaataaacaaacaaataatagtcaaaagagacaagtcataaaaaatataactggaagacaaaccttaagaaatattactcctaattgttatcaaataagttaatatcaaaacaatggtagtgtaatttctgcttttcaatttggccaataaaaaagaagaaacaaaagcaaaacatgaaaaggaaaacaaaacaaaaacccaggtaaataatgcattatgctgatgaaggagatccatgagacagatacttaacacaaatctcagaacatggtatctttcaataaatgttgagtaattgaatatttcatatacagctaatagtgaaattaaaagcccagctacatgtggatggtttctttgtctctaaggtgggaatagctatttgcttctccttggtcagagatgatcacaggatttttcatttatttatttgtttgtttgtttgtttgttttttgagacagggcctcactcggtcatccacgccagagtgcagtggcgcaaccacagctcactgcagcctcaacctcctgggctcaagccgtcctcccacctcagcctccaaagtagcatgcaccaccacatccagctatttaaaaaaatttttggccaggcgcggtgtctcacactgtaatcccagcactttgggaggctgaggtaggtggatcacctgaggtcaggagttcaagaccagcctggccaacatggtgaaaccctgtctctactaaaaatataaataattagccaggcatggtggcaggtgcctgtaatcccagctacttgggaggctgaggcaggggaatcgcttgagcccgggaggcagaggtcacagtgagccgagattgcgccattgcactccagcctgggcaacaagagcgaaactccgtatcaaaaaaaaaaacatcatttttttttcttttttttagtagggacagcaggatctcactatgttgaccagactgatcacaggacttttttagccacagtcccaacatgttacccagactgtgcaggcccagcagccatacccactctgaacttcctcatccttttctcctcctgagtccagatctgctccctcatcaccttctcatcagtcctcacttcatatctctcttggatttaagactgtccaaatgtatcatttcaaattctcaaacactacaaagtcatagaaaattaaagctggaactttaaagatcatctcttcattttacatatttaaaaaatgggctaaaggaagaattatttgagcaaagccacgcagaaagtaagaggtcaaaccaggtctgggtttctttccagtttattgttggtggctttctttccactactgtggcctgggccaggagaagggtgaaaaaataaggttgttggctccactggaggccataacttgttttaaaagttcttggaatagtcaattcagaaatggtgagtgacagatgtggtttggctgtataaagtaatttacatcatctacttactccatatatggatcattttaccacatgataatgtatacacatattctgcatatatgcaaataaaatgaccaatatgcatacttattgtaatgaggtatagctataaaatattcatagaggcaccatgttgaatttaaaaagagctttaaagttgagaattttgcatcctagtttgtttttttttaacctattgtgtgctttttgccaaatcaattatactatccaggtctactttaccaatctgaatattgcagttttctgaggtcatgttcaattccaactcctctcagcttctagtaaaaaacctgagtataatttgtcaagtttacctgattggtagtatatgttttcacgtagcaaagctttcacattttgctgcttctgtaagcctggttgttggtacttaacattaaaaagtcagaactactaattatttatttcttgaagaaaaaagaaaacattgtaacttttctaaaatgattcggtgtgacatgatcaccagagtgacatcccattacttttgccacattgtattggttagaagcaagtcacaggtcctgcccatacataagggcaggggattatacaagggcatgacaacagtggccagaaattttggagaccatcttagaattctgcttactacagactgtcatgaagaaagcatgtttgcagacataaagaagagattcttacatgtcattcaacatactgtcaattcttcaagtttatagattaaacccctgaaataacttttttaatacccacaaagatactgtctcttcacttggtattaccccgccccccacctagttactagtggtgttggtgacaaattctaaactttaagagcctttttaaaataatagctctttgcattagcttagtgccaagttgtttcatctgaaattgctaaatcttgaagatttttttcattttatcaccttttttccactgaaattcatgatactaaattccacataatttcccctatattcaccacacatattttttctttcatttaacatcatcttcttaaatttgggcatgcatttccccaaattttgattcaattatggttttccaatacattttcaaaattttttaaattttcatttatgtatttatttattttagaaatgggatccagctataatgctgaaattgttcccaaacttataagttcaagcaattctcctgcctcagcttctcaagtagctgggactatagggagttttccaatcttttaaataatattttaatcaaattttttcatgtatcatgcataaattagatgaaagtggaacttccctgcttgaattaaagccttcagccacttaaggctggagtgagatctctggagaacattggctggaaacacctcttctgtcatattagtaggacagatgcctttcattgcaacatacatactgaatgttggtataattgttgattgaaatttagctgtacaacttatgatctgcagatgatagactttctctaagttctaaagtacattaaagactaagcatatagcattttagaaataatttatacaaattatgtgtttaagcttaatgcctttcttgtttttataaacttactatcatctttgagaaggtctgggtgacttttatattaatgtttttatttattttaatctattgactatgtaaaaggtttactatttgtttaatttaattttaatttctgtattgtattccttatttaacatgccaatcaaaactcttgttgttccccatgttttggctaaaaacagtttagttcggtcacttgtgaacttattttattttgtttgtttttctttcctttcttacactaatgctcatttatttacattatatataaatttctagaaaaagcactaacacttctctcaggttatcatagtttatctagagtaattcattgacataagccccactgcttatggtttaatctttctaatagtcacatgtcactttttctgatgttattatgatttgaaagtaggggaaaattcttcttactattatgagaatcagggtctatcataagggttagccatgatgtggcttaccagcctccctttgcaaaatgctgacttgcgaagtagagcttaagccaaatatgaataccattttttaaatctaatttttaatcatatattgttcctgaaagatgtatacacttctctggatcccaaacattcaactgctccatttatttacaaaataaaaatgacatgtataaaaatgtcactttaggtgccttgttccagcatgggttcattgtttggagtgctacatcaatgttttagcatgagctgcacttgtaattcatcatctattatttctgtttagttttttttttcccccagaggacaaatatcttaacgaatcatataaagcaacctgaatttagtatcttctcagaaatttatatttgtaactatttaaaaagtaaatattcacacctaattttatggttttttaatgtacatgtatctatgtgggggatttaccggtgttacgtttattcaatgattttgacttagtttttataaatacaacaacactttttaaatgcatttttcaaattctacaagattaaattacacacttacagtattttagaatagtgtaaattgatttgaaaataaacataacttattcttgataagcataccattcagctggtgaaaatagaaatgtgcagatatcctaggcagtagccaacgacctgagcaatttttgcacatcaaggtcatcagttattgtattttactgagaaaactgcaagtgtcattaattttgtaagtaagtccattagaatgaagagaagttataataaaatatatgcccaaagtagtccctgtaaagtgttgaaaggggtttaaataagagagcaattattactcttccgcaatgatgggaggaaaaatcactaggagaaaaagtcagagaacattaagaagttcaccttgggccgggcgtggtggatcacgcctgtaatcccagcactttgggaggccgagacgggtggatcacaagatcaggagatcgagaccctcctggctaacatggtgaaaccctgtctctactaaaaatacaaaaaattagccgggcatggtggcgggcgcctgtagtcccagctacttgggaggctgaggcaggagaatggcatgagcctgggaggcggagcttgcagtgagccgagatcgcgccactgcactccagcgtggacgacagagcgagactctgtctcaaaaaaaaaaaaaaaagaagttcaccttgattaacatacagcttctatctgtgaaagacaaatttgggagatttggttggaactctaagttgaaaacaaaaacttcatccatcactaatatctgaaacagaggtattcatctattcattcattcaccaaatgtttgttagtcattcaccatacatacactaagctttgtggaccacaaaggtctaagtcacatgaggctagaatgaggatggccatttttatttatagttaaacaaagaaaaggtaacaagaataaacagaaatttcataggaatttggaatctgactgagaaaatagttatttcagagataaaataaggaaaactgacttttgattactgccaatgtttgaaataagtaaaagtaaacaaaaaaatttgagtctgggtgctgaaagagtgatattgcagaatcaggaatttggttaaagaagtatgtttaagattgtttaaaaagtcattacttagattgagagcatactaaatttgagtattattaggcacacctccactttcctacagtagttcagacagactgtagaatatacttatattgcttaaaaaatgttttaaaattaatttgtaaacatgtagaagatcacttgtctagacctgcgaactttccaagtgcagatattccattggaatgctaataagggcatttgatcatcttttaaatcaccttaaaatttctgaaatatggagtgactccaagctctatgcattaaaaaaagaaagcacacttagaagtatattgtatccatccattgtgactatctttggctttgccattaatgttttacaatttggagaagtaacactattcaccaattcctggatctacagcccacccatggtcccagtttgtttataaagtagtcctcatgaaatagcaaagttattcagaaacatttaacatattttaaatataagataatttagagttcatattatcttgtaatgttcatacaatcatacagttcatacaatcttatgttattaatatccactacattcttcaagagttaccaatgtaatatgggtacttacaagcgcttgctcatgtacttaaaattagtagtaagttttactttcctaacatttagtagacagggtagagttcgtgtatgtccaattgcatgccaactcttatattgttcaattttccattttgatgttattctcatattgtgttacatcatcattacactatcttggttgagtaactcaaagggataaaataggaatggaaatcatataaacttaaaactcaggtttttttgtgaattgagactctaatgaaaaaacatcagaagaaccataaatctacatgtatttttaaaaagtaattctgtaagagtgaccatcttaggtaagcatcctaaaaataaatgtctaaataagaaatataagatgaatatctgaatattaaaacttattgaactgttgatctatttgtcttcttcagagaatgccattttgaaaatgtgtcaaaaagtaacataaggtcttttgggttctcgtaattttcacagaaaatagtcacaacttaggtaagtcagataagtctttgtcttagaaaaagtaaaaaacaatgctttaattcataaagtgaataagtggtttcacttaaaggcagaataaaaatcaatgcttcagaggtatcattaaaagtagtattcctcataagaaaacaaatatggttggcaaaaaagcaaaactagttattggcaatactttattatcagtgtatcagtatactttaatatcatgcaataacaattgcatgatattactagtaatcaaggaagaacaatttttatgtacatgttcatgccaggatctaattcaccttacaattggataaaacacttatgtgcaaagaatcaatcatttatctgtggcacttacatgaagattaattactgagcaactttttgttctgcttattactgacaacctatactgatgaagagtagatttttatgctaatattctattattttgtgagtcttggtatagactggaaaactatgtggggatcagacctagcagataacaattatatatatagaaggtaggtttgctattataattctttgtacctgaatgcctctccacacaaatttattcacaggaaataatatcctttctagttcttaattaattttggcgaaaattgttaaaattctcaaattccactctctaattgtttcatgaagaatgggcatttgcttttctacagtgaagaacaatgggtgatgaaatgttgctcacatggtttttataaagtatgtaatgaatttcattatgtcccggatagtgccaatcaaggtcatttttaaaatatgaaggcacttgttgaagtgacaagtcttagcctatcactttacggccaaatgatgaatctgtgtaatgttgaggataagacataagaatttcttcagaagatcatgtgatgttatgagtagtttgattgagaatgtgactaacgcctgccactaggtgattctcttcgtttgtcagaggaagaaaccaatgacttattcctggatatgttttaaattcagtgcaaatgctagaacagaacacaaagatagtcaaagctaaatgcaacccgaaagtagattatgatctatttactactgtctatcatgttgaacaatcaacttttcagcttctgagcatagtatgcaggcctatttaatgtgttagaatgttgaaattagtcttcagtgagaaatattttataatttcaaagatcatcttcaacctgaattcacagagctatgacagagctacatgccattataggtcctaattttctgtaactgtcaggtaagtttttctattttaagaaattaaaaggaaaggaaagaaggcaactagatatggacccagacatataagctatccttcttcaagcaaatacaaatatttcggtgaaacttttactgaaatatcaacactctcctatgtatgtgaaattcatattaacaattttaatattgtgtttaatttttactatttacaatatcgtgataacgggttgaatggcttgctgctgtgtgcactggaatgcctattgtcgggtctacataagcctgaagtttagaaaatactgtactagatttgatactccaataaggacaatagatatttcagttatcagtgaatactcagctggtagcatattgttggtgttcaaaaaatattagttgaataaattaatgaattaattacacttgccaaaaacttataagccagtttgctggtcttcagaaagagcgtcatgagtttttcctatcttaatatgttagttaatattttctattatctctgccatctttgtcttctgagaactctagaaagagaaatgtgcatcccatcactattgtcaaaatatgaaggatattgttattgttgttgttgtttaactagattttgttctttcagcttcacatggcacattaacataaacatgatttgtcagaggaaactgaattttcccacacactatttatctcattttacaacacttcaggctctaaaatacttgccagaaattgaagatgtctatttaatatctctaaggttgtttattgatgtaatttttgacatattttataacaatttctctccaaatgaccacatttatagtaaataactctgaacaccttaaccatgaatcttagtaacataagtgaaaacaacaggtagtatctgtacacacaggtaagacattgttaaagaaatatgtaaagtcaaacattaaactgcagaataacctaacgacattattttcttctaaggtaagctttctctttctctcctgctccttttccctttctcttcctccctcttctcttccctgtgtcccctgcttctctaatccacaaggcagtgttttgctcagtacatgtggctgatggccttttgtaacctcataacatagcaatctgatttcaagcttacattgtgaattatggtaaaagggttagcacagacaatgattttcttatttcttccccttattcaatctctctttttctctaaaaatatctctacctcaagaagaataaaaaacaaattcatagtaataatcctttcttggcaggcaacttattaccaaaattaaaggactttactttctatgtccatctcacttacagaaactgaatgaaagcagtagctcatcagaaggtagcactgtggacatcggcgcacctgtagaagaacagcccgtagtggaacctgaagaaactcttgaaccagaagcttgtttcactgaaggtaaagaaaagaatcctaatgttaatctttcatttggagtgcagcttgtttagctgttggtcagctaaaataaatcacatataataaaatggcactttgtaatagatataattcaatcacctctaatatgttgacagacaaaaaaacttaaagtctagtgtcatgctttgattatatctgcccaatatgtggaagagattaagaacatggaggaaaaatattttagtaccatcaaaacttctaacttatatttttaaagtaagtagtgatttcacaataagttggcatcttagacactactgaaaatgatcctacacagaatttgccttaaaagtaactttctgactgctaaaactaaacggattgctagtgctaaattgtgtttctttaatgaaaacatcagtttccaataattgttggatgaaatggggaataaatactgccctagaaactctatacctaaacagtcactagttgagcctggtgcagtatgggcaatagatagaagagatatgtttttcctacaactaatatttcctgtggtctgatatccctgggaatttgaggtttggaaatatgcatactttctatttgatgttattttttcataggcctagtataccttcagtcttcttctattccctatacaaatcatgcatatcacttaatgtccttttcaaaactgccctcatctgaccatggtggaacagcagcttgttcctctgaatggctgtaagaagtatttccatttaatttacccgataatatacattatcctatgaaaactctttctctttttgaacagttacatgttacttttctatcttttttctcttcttttcctccattccttcactccttagttccttctttcctccttcccttcttttcttcctccttttctccattcattccctttctcctttttcttcaaacatatcttttcctccttttttttttttttgccttggagcagggatcctgtatttatatctttttcttttttttttttttgaaaaccagattttttttggtaaggttcttgggagtaaggtacatgttcggtgcctttttgtgtgtgtacctaagtgcctttgtgcttacaatggtactcagaagtacctgttgattgcatttagactaagtgaaagcaatgtgtgaggaaggcaatgaaaagtcaaagccaaatcatacaaattagcttttagtgaactaaattaattatgcaaattacataagcccttctaggcacatcacaaaaaatcctaaatggtaacaaatgcagcattgttggaagagatctacatttttactctatatcctttataaaagggaagttttctgcttaacattattaatgtccaatttaaagttcttttctggagtgacttcaacagagtgcaacaggatgatatggtacaaggaaaagctacctcacatttatttgtaatttacaaagtttctcttggacaataacactgtgaggcagacaaagcaaatactgtccgctagggcatcatttaaagtggctgaatgtttccacaacttcacacagctgatgaatgtgctcttactactctaggcttagagagctatgctagcaagacagagatgagcatagtaataaaaagacaagacaaggacattgctaaaggatattatggaagcagagacactttatctacttttatttcaacactttctgcaggctgtgtacaaagattcaagtgttgtcaaatcaatgtggaagaaggcagaggaaaacaatggtggaacctgagaaggacgtgtttccgaatagttgaacataactggtttgagaccttcattgttttcatgattctccttagtagtggtgctctggtgagtgagattaagaaaaggtgatacagcactaatttttagaacactctaatactgatgacttattaatcctttgtttcattgtcttagtatccaatgcatttttaattatcccaccttgtatcttctatagatttactctataactctatatttctggattaacttttactatgtatgtaaatataattttaagaagctaatcattaatttttgcttactattaaatagcccagaaagtgtagcccttcagcttattcattaacaccaaaggatgtgaatattcaattactcattgtttaattaaaaaaagttattggaagccctgggatgggaatatatattctctgagttctgacgaattttgtttctaaatggtatttattattattatctcttgatggacagtagtaagtccttagggaccacttctctgtctttttccaattgtgtgctcactatttttgtgctacttaagaaataatgaaagttggtaacaataaatattgggcagcatgcccaagagattcatttaaggatgggtttatctttataacaaggtgttcacttcatcagaatcaatttatttttataggaaaattatttataaaactataaataaaacatacagatagcattttagggaaacactaacacaaaggaaataccatagatagaaaaagagaaagaaagagagagaaacagagtttgcctgattgaaatgcaagtgtgttacaagtttaactttttataattctaacacagacatatatttacctgtaatttttctgaagaaagaaatattttctacacgctactttagaagtctttcaccgttgaaaggaatgcatacttagaagtgaaacatcgagaggttcataatagaagaatatatattagaatctaaatgaaaagtaatttcatttaatcattgtcacactgtatgtctgttgagaaacataaattattaaaaaaataacaaacaacaacaaaaaagaagcagatgaacccaactatttagtcaaattggagactgcgaagatcaatcacaatttctgctttacaaaagcacaagctttcattatttcttcaacctcctttctatagcaaaaaaaaaaaaaaaaaaaaaaagccaatagaaggaagcaaaataaatgaaacaaaaggaatacataacttcaagttgggatggccacatcaggatacaacatcaagaactatttcctgactaagtcaaattaattcattggaatcatacttttctttttcttccaccaatagtctttcccctgattaaataagtaaaagacctttgcgaggaaaaaaaaaaagtaacagtaactactgtttctctgccctcctattccaatgaaatgtcatatgcatatgattaattttttaaatagcttatggagtataattatttttgaaagctaataatgtgtaacattttctttataggcatttgaagatatatatattgatcagcgaaagacgattaagacgatgttggaatatgctgacaaggttttcacttacattttcattctggaaatgcttctaaaatgggtggcatatggctatcaaacatatttcaccaatgcctggtgttggctggacttcttaattgttgatgtaggtatcgttcatatttttgtctctgttcaaggtagcttgtcttatttatattcaaattctacaatagtgagtctcagaccactatgttatgttgacagactataataaccactaaacgcatatatgcaatgagagtgtcatttctggaagacaagggctaatttaattctgatttaaaataacattgggggatggaatctatggtagttctctagttatctgagaaagctaaagaaaaaacaacacatcagcagtttgtagggaaagtagtgaaacagggaacttaaaaggtatgttgcttaaatcctttatgttgtgagtcagacagactcaagtttgtatcccaggaccatcacttgttgattttgtgccactgagttagtttcttggccccaaactcttgacccccaacttgtacagtattaaaggcactcaataaaaacagtgatgctgttgggtgctacagtcatttaaaaaaattctatttgatctactcaaactttttaaagagtagatttaaatagtagattatatggattatgacttcattataggaagtaaggtatgtagagtgtagggaaagagaagtctactttattcagcagtgaaagttttcctagaatgtaaactagatttacggcaagcccaaacagccctttatagaatttgtatccccaatgattaatactgcatttactgaaaacaagtgactgacttcaagatgtgttaaagttaagcatcatgttttctattatcaacttggcaaaatttcagctttagaaagctacagcaaaactatcctgatttcatattgagtagttttaccccaaaaagatactaacccagcaagactatgagggtttcttgtggaaagtgtttcttgagctaagaatcatgaatgcattatatttccttgaccctgaacaaaggcaaaactaagctatttaaaaaacttaacaatgttactgtgaaatgagccaattgagagaaatctcaaaggaagaattgctttatcacatcaataagggagaaagtttgtaatattgaggtttcaagcattagaagttacattatgtttcaatttcagaattgtttacactataatttaaatctaaatgtaatttaaccctttaaaacttcctgaggttttttttctattatgtttattacacatcagctttttcatgaaagttttttagtgtcatttttgaaacattttaagaaaataattttatagaaaattttgtagtttgtaacaataatatctttattttcaatgttctgttttaattctttaatgaagatcagtatagcactctataaaaggactaattacagcacatctcaaataatgtaagaagtatattaaatatgatacaattaatacattttaaaaaagaaaagtttacaaaaatagtgtgcagtatatgcggaattctccaatactagcatctttctaaccaaataagttataatcttccttgaatacaaagttttagtaaattccttatttagagaattagatacgtaagatttttcctttagtcatgtatattacctacaaatgaatacaaatgaagtaaatttattgctgtgtgtatttggtaaatattcattgattggccctttggggccagatatcatggcagaagccataaattagtataaaaaattttgaggaagttttgtaatggaatttctttctaagttacttatgtaaaaataaacaagtgaaaatgcaaggtattaccaagacaaaatatcaccttcaaaactgatgtagttcattaatttctgagaataccaattatgattagttgaatatactgatgcacagtagaaacttcctgctttttttagtacaacagcagtttttgtattttataactagctacaagaaaagccttaaattcaacagcaacttaataaaataaaggaagaatagccaatgaatttttttccaccttgatttcttcttttattttactaatttttactttgctttttttttctatttattcagttgataaattatatgacatttaaggattttttttcaactagcttttaattaatgtcctaaaaattacatcctttacatcaaactgaaattatttctttaattttttaaaggtttactgaaaatgtctgaacatttatcctctgaataattataagcaaaaattatacttgttgtattatatagcaactacacattgaatgatgattctgtttattaattgttattatttttgtgtgtgcaggtttcattggtcagtttaacagcaaatgccttgggttactcagaacttggagccatcaaatctctcaggacactaagagctctgagacctctaagagccttatctcgatttgaagggatgagggtaagaaaaatgaaagaacctgaagtattgtatatagccaaaattaaactaaattaaatttagaaaaaggaaaatctatgcatgcaaaaggaatggcaaattcttgcaaaattgctactttattgttttatctgttgcatatttacttctaggtgatatgcaagagaaataggcctctcttgaaatgatataatatcatttatctgctgtgcttatttaaatgactttatttcctaatccatcttgggagtttccttacaaatctatatacaaaaaaaagctgatgcattattaaagtactatgtgtaatgatataatggtaatctaaagtaaattctatatcaggtacttattctttgtgatgatatactgtacttaacgagttttcctgaaaataatgtgaatcacacatgtgcctaagtatgagtgttaagaaaaaaatgaaaggagttgttaaaacttttgtctgtataatgccaaagtttgcattatttgaatatattcaagattagatggttagatattaagtgttgactgaatttataaaactagtaatactaacttaaagattacatacaaatccacatcatttttataacaataaagtaaaacacttataatgaacagaaaatataattttgactcattactataggtaatttatacattaaccttaacttgcatcttattggtcagagtcacacaaaatgttattttatccttttcaaagatgcaataatcattttccatcatgcataacagattagaaattttgccattattgacttattttccatgcctttttttacggcatgaagcattagtttatagatatataatataaaaaattagttctgcttttttttaaaaaaaaatattatcaaaacaaaacactgaattgtgtgattccaatagaaaaacactgctctttcacctcctaaggtgtagttacttttatggaaactaagctgtattgtagacttccatttgcactttgtagattgtttatagccttatgttctcttctcaagtcttattataaatgtcactttgtaagaacgtaggacttgtcttcgatttccctaacatatatgaaaactttgtcctcattatcgacaactcagaacaatataatacaagtagtcctcttttatttctcacagagagcctcaaattttcaccaaaatgttaacagaaattatctctggggtgtataagaattaagtctgttttccaattaaatgtcactttgttttgtttcagactggcagtttcagttctggagaaaaaaaatgtcatttgtgtacattctacttgaaaacatgttgcctgaatcaaaataatatattttatatggcttgtgaaatctgaacaatgctaaacatttgaaaatattataaaccttttacatttgaccatttgaaagtttattaaattcattggtcaagtgctcagatatttccatacattacacttcatttctataaaaaagctgatcttatcggtatacttttaattttctcagaaataaccatatctataattattaatcaataatgccttttatattaaaagaggttagtttttgaaacttggagttttagacataaaatccttataaatgctgatagtgatataactaatagtttaaatggtcagatttatgaatatggctctattcctcataatgacaacatacacacagcactaaaatgactaatctcttcaatacgtgtttggcattgtagagtcaaaataacgttataattgattctattttttatacttctagtgtttggatattttattttgtaaaaatataatcatgaatgatggtgaggttggatataagaatgatgattatgattgggaagtgagatttgaacatgctcagaaactctcatttaattctttgccctagcagcataaaatcacaatagctgcgtcaaagcgtaactcaggcactcattttatttttgttgttctgttattttttcaaagcatgtgcttttatgcaacattactgaataaagcatgttgtacagtgcttgataagaagttagaaagtaacaaataaattatcatcacgttgcactttgtgttttgcatgttttatgcacatttctggctgacagcttttaaacatttattgtatttcaaatttccagtccaaatttttcaacttgtaaaattaaactgagtgaattgatgtcgtgaatatctagggtaaaataaaatttgtgtttaaatttgtatttttaatttcctaacctaggaaatcttaaataccttctttttcaaaagaactcaagtcttaatggatagggaaacagacggagagcatcatgaacaaaaagtaacaccaaatgttctgtcatatcagatttctaactaataacaaactatatatttctattttgtataggataatcttgctccaacttggatggggtggagcgctggttcctcccctgagccctttattatgggtactgtattaccccttttgctacctttaatccttgcactgtgacttatgtgtagtggggtgagggagggattgggaagggtactattattgcaccacagtagggaaaatacattatttacatcctaatcccctcttttcaattgtcttaaatttcatttgaaaaaaaaaaaaacctttatgaatttaccctctgtggattttaaccccaatggttgatatctttattaagtttcattgaatatgatttagttatgtgtatatggagttatccatctttggggagattactggattggtgagggcgggggaccctggtgtagaatgattatgtgaaaaaacaatttaacttgttaagctcatgatactgtttgaggcatacagcccctgctgtttagtacattggtctgggtcctgaaaattaccagttagataccatcagttgattattgatatgtatgagcagatactagggtgcaatatttcaggtttcataagactggtattgattgtgaccactctcattttttattgtgtaagttcatatggggttattttcaaaatgttaacaaggcaaaaatatattaagaaatagttgaataagcacatgtgaattgtgttgtaaacaaaaagttagaataaaaaaatccacttatttgaattatgcagaatagaatacatacctagaaataaaacaaaaacgtcttatcatgagtattaagataaaatttaaggcataaactcacttcttagaataagtaactcccaactaactttctaggattttaaaacataacacagtgaaaacatacataaacataactctacattttatttattcttaaagtttaagtgtattatacaagaagaagagtttatattcgagagacagaaaaagtcagaatttttgtttggatcaccaatatatcatagcttacaaaaaaactgtcttaattaaaacccacaacataatttttttagatttttaagaaagattctattattcttctttatacttaaaaatggatgattcctactttgcccacttttatttttattcacatagattttctttatttctattagagaagcactagaattcatgaatagtgttgatttgaagttcaaagtaattaattcagataaaaagacatttctgcatgtatgaaaatttctaatgtgaatttgcatatttaattatcaatccttcatttagtgtagacttatttttaaaaatgcaggtaatgaaccagaaatagaattggttgtgctagagtagagaaactttatttgatgattgttttgaaaaaaaagcttctgagaagaaacaacctctagtacagtattaattcattaagatagctcctttctcagacatttcctttcatgtagcctgaaagttcaatttgaaatttgttctttccaatttattcagactaattctgcctactttcttccccccataagaaccaattactgcagctttattgagactgaaaaaagttaatacacctccttctttgctgaaccaaggaatggcttggaactcttgggaaaagacaatcttttctatgatctttcattgtctaatttaatacatcataaatatgactatagctttgtataataaactccccaatactgtgccagatgttttctaagataaagttattttatgttcacaaaaaaaataaaacttttctctgggccaaatgtatgccaactttgcaaatcatatcctgaagtgcactgctgcagagtacatgcttgcgtcataaattccatagagttcgctttaactctaaatcaatccccagtttcaaagtaaacctctcaaacatattacctaagcacaaacttctccctgtgctcagttccttaattattctcatcccatattcagaaataacatttaaaaattatgctttgatcaataaatactaatctaaactttgcttcattaacccattcatttttgtcaaccattattttattcctatattcaaagctctctggtatgttcttatattcaagacactcaaggccctggaagattcacgaacatatgtttgcatcttaaatttttagaaaatcttacaatctgtcaggattacactgaactctagtacagagtaatatgggtaccagataagtgggagcaactcttccacgtagactggaaacagcactaaatgctatttataggctactttctgaacttaacttgttttaacctcatttttctcatatgccaaatgagaacgcaatactgaattatctgtacagttctgttcagtactagaattctgattcttgaattcaaaggggaaaacattcctctttattttggaggctaaactgggggacaaagttaggctccatgaaagaagtgctatttgaactaaagcctttaagaggggagagtatttcagaagaggagctattagacaaggaatttcaatgtaaatggcatctcaatcacctggcaattatattagcacacggttattatattaattgaagtggcatgaagtatagatgaccagggaagttaaaactggaaatatagattgtggagtgatgtgaataccaaggtaagaaaaatatttgttagttaccagagagccaataaataactttcaagtgggacttggggaagattaattcatcttacatagattaaatgaaggagaaggttaggagacagatgacagtgcaagtatgaaataacagagggcagttctaggtggtgactgtgagaatggaaaagaggtggcaaagctgagaaacgtttcaaagaaaaaatgtgagacaggtaatgtgaaaagaaaatcgagaaataggtatagataatcagtgttctgctcatactctaaattgggtgttgaaggcaaaatacgtattttaattagtactctgtgtatacacactagaaacagcattgtaatctggatagtggacaaaatattcagaaaagaagggaaatagtaacttgatttcaatttccaaatctctaatctgaaagaaatctaattctattcatccatttaaaataaattatataacgagaatttatgaagtccattgtattaatgcagacagtcagatgagataaggcaaagtgtcacgtgtcagcttggtagttgcatcggccacatcatttggttctgcctggataactcaaccaaattaatttttcatactcatcccctccacctttgtcattactggtattcttattttctttggcccacttatcacactgttttatgttccccagaaggcctagagttctttacaggctttaaacagggatcagaagtataagaaattggctcatgtatttttttttcagacaggcagttaaaaaaaattgttctaaaaatacactggcatcaaatggcaaatagaagatgttttgacgactacttccattggatcagactgacaagaataatacaagcacataggtggaattaaacttagctattaatgtccaagtttgaggcagctgccccttataagcattttagggtctgtttttagcttccctcttagccactcctgtgcagctccagtgggaggtatggaggaaaaagcaaggaagccatccctatgttgtttccaaacatgaacactcaagatttttaactagtggtccagaagtaaagagggggaaaacatccttctatagaaaaaaaaaaaagtagataataattgaacacagaacttcatgtgatcacatcagatttgagaactatgtatggcatccctctttttcttattttcctaagaaatgatttctattatgtttcatttgaaataagttttttgaattaaactcagtaaatgaaacaactgacatgactggagcttgaaataaacgatgtgatgatctaatgaaatacataatgcaaattgtcttgcttcttatgcaaaaattattagtcatagcaatgcatgaataattaaagacaattatattaggtatttaataatattttttatatttatcatctgaatttttaagttattttaaaaatatattggtcaaatcaactcaggtccaaatgttttagttttgttctttaatatattgcctttttaaaatgagttaaacttctgtataggctttttaacttttctttattctgataacacaattctgacttcatctggcagcaagttcctctgattttccttttcctttaaccttttaatgcttctccctcccttttttttaaaaacatttttgtttcatttcttggttatattgcctatagttgttttcctaagtgtattgcttaagaaaaaaaaatgaattttaagatttttttgaaccttgcttttacatatcctagaataaatagcattgatagaaaaaaagaatggaaagaccagagattactaggggaattttttttctttattaacagataagaattctgacttttctttttttccatttgtgtattaggtggttgtgaatgcccttttaggagcaattccatccatcatgaatgtgcttctggtttgtcttatattctggctaattttcagcatcatgggcgtaaatttgtttgctggcaaattctaccactgtattaacaccacaactggtgacaggtttgacatcgaagacgtgaataatcatactgattgcctaaaactaatagaaagaaatgagactgctcgatggaaaaatgtgaaagtaaactttgataatgtaggatttgggtatctctctttgcttcaagttgtaagtgaacactattttctctgaatatttttattgtttggaataataacaaaataatgacatacatctattatttagttcctaagaaaaagtatatatttctttctatttaaaaaatttcaatttgttagtacaagtttatgagcccagatgggtgaaaactttattacatgtaaggactgcagatatagtcagtagtgaccagaccaaacaaagacctctttgtcttttttcttgtattttctttgtttctatattgctctttctatataaacaagtaaattaataaatcagtaactttgccattttttattgtgattagaaggcttgtttccctgatcagctaccaaaacggaatacacttaacagatagatcatcttccaaaacacatacacttaatacacctaattggattgtaattaattggtagggattaactgctgctgagttctattatttggatcaaagtttggggaatgacatttaggttttagaattagaaccaggcatagcggtgggtgccactgcactccagcctgggcaacaaagagtgaaactccatctaaaaaaaaaaaaaaaaaaaaaaaaaaagagagagaattatacctggaagcaactagaaacaaaaatgaagcaaaagcagctgttgtcttatctcaaatgacctttcagcaccattatttaagtctattttgtcctaaatattaatgttttcactactgggtctctaagactgggtctccaagaaaaatagtttataatttatatttaaaaaaggtagccctttaaacattttcatttgattctgcttctagctgcagcagcaccacaacaaagcagggaataaaataagagtccagattatttgaaaactcagcttaaaaaaaaaagactgtacaatttctttcttcccttgtggttctacagtgtctttagggcagaaatcaatttttgctcaccaaggttatgatttcaactgttcaaccagaagactaactaagcaaacaaaccaacactggtcaaaacccttttgcagctcttccagaggtgtcaactatgattactttacaaattcttcccacctttttgttggaaattcttatttctcttcttaacatttttgatctactctgaataatattcatgcaattaaaatagagattttttaaatctggtaagaacgggtggggcatggtggctcatgcctgtaatcctagctctttgggaggccaaagctggaggattgcttgaggccaggagatctagaccagtggaggcaacatagtgagaccttgtctctaaagacaaataaaaacaaaagttaaaaaaaatctagcaagaatgctttctttaaaaattgtcgacacatgatgatatttatttgggcacttattctaacccttaactaatgaccatacttgtaaatatcactaaagcaaccacctcctccataaatttgaggaaagaaaaattaaaaattttaagcaatcctttaaaatgtaagcaattctgaaaatacagtgatggtcctgcacaaagctattaaataaattctgccaccaaaaaaaaaaaaaaccctctaaaatactgcttcacatttggcttttatagaatcacctctctattgattagtaagtcattgaaatggtcccttgaatcacttttttgtttctataccaagtgtgtaatggaattgttctatcattgacctaagccactgacatatttaacacatctctctctctttcatatacaaaagccttgtactttctagctaggaatcattactttcttttcactgctgtcacccacacccttgcttagcttttgtctcatgaaatccaccacctcacaagggttagcgttatcagaacaaagatatattgtgatacagacgctaaaggctttgctaacctccctcactagctaagggataatccttttaggcccacaggactcaaaattcatgccccaggacaagtaacaaattactgtaagttgttagcctgagtatccttcatcctgtgagtgccaagtgtctgctgtactattgttcacatttcttattcatattaacttattatcttcaagaatttcagaaagaaaatagaatagggggttatttctaaatgttttgatgcaaaattcttaaaaggattgtatttctaaatgttttgctccaaaattcttaaaaggatagttgcaaccctgcctttatgtatcctggtactatccttacattgttacaataaagagaaatcttaaattttcaatcactttgatcaccctgcagataaattttagcagatgatccttagatctaagttgtaattatttggctgatagagctattgacccatgtgttggcatttgtaagactttctaaaaagtgctccagcaaattttaatactgctttaaatatcatgacaccatgaactctaaagaattgaaaagtgcttgtgagttggttatcaaggggaggatatatcaaatggccattttgttcaatatgtgttctagaaatgaaaagccatactaaaatactgtcttggtccaaaatctgtgtaaaatttgttttgaaatgtctttcaaaaatattcccttttgaaaattatatcagtaagaatatttattaaacatcaggtctaaattatttttactccaaagtaaaacatgcatgtccttcttaataggccacattcaaaggatggatggatataatgtatgcagcagttgattccagaaatgtaagtattccttgtattctaagtctttttacaatattgatcaggtggtaaaattaatcgaataaagcataaacgaccaaatgaaatgattctatcttgatttaaaatatttgggaaaaagtgtgacaggtaaatattcaagcatagcaatgtttatcagaaagatcttactaagataattcaacacatgaattattttgaaaaccaaagtaacaaattcatgtatttagtaactaacaaatattttccttttaaatattttctattttgtttcctatttatccacacattatataattagtagtaattttagtgtgcgggaccaaatttgaagaaatatatgatttctatcattagttgaaatgtatagtcaccctccatattagacagcatgaaaagttatggatattgttctgtcagccttagattactgtgatgatttctttcaaataatcctattttaaaccatataaaagaatgtttttatatgatttttgcattcataaattgactggagcctttactaatgttgcaatcacttctgtgaccaacagattcctttaagtgtatatcaccaaaatataaacctattttatttgatttcagcctctttcaggctccaagacctaaactgaaattcatgctcattccaactttgctcttcaaaactttgtaagatgtagtattttctatcaaccttcatctttctattctcaagtcccatctgtttagtttatcatcataaagaataacctctgtccctgcagtcttcaacctgatagagtttgatgttatctttcttgaagtgcatttcaaatagctttagttttatgttgaatatcattttgataacctccagagttgttttatttcactttgtcacttatattttgttttgtgtgtttagttggtttttaactttgttgcttttagaaggacatgaatctgttaaaaaactatagtgacctttcagtccattactaaattataagtaatagcccatttgttttaagcacaatctaggaattaaataatcaactgaggatttataaaatggactgtccatctatgtctacttaatctgaaaaattcaataactgttactttagggtaaagtgccaaacatctgaatgaacaactcatttcttatttgctacattatttcatgcatacatgaatgactgtttctaaacctagcctgtagatatttacagttttaaattttttattaataattttaaaattactaaaattcagtaccttaaaagcttgcaaatttagcacattactgttcaaatggaagaatattatttgtatttatagttataaaaatagattttagaatgtcataatagccatatttttcacatgaaaacatccctaaaggccaatttcagggatgttttttagaggtagttgattcagaaaaaaaaaaaatgctgacatattagtaagaataattttttctattgttatgaaaaagcaccagtgacatttccagcactaaaatgtatggtaatattttacaaaatattcccctttggtaggtggaactccagcctaagtatgaagaaagtctgtacatgtatctttactttgttattttcatcatctttgggtccttcttcaccttgaacctgtttattggtgtcatcatagataatttcaaccagcagaaaaagaagataagtatttctaatattttctctcccactgagatagaaaattattccttggagtgttttctctgccaaatgagtacttgaatttagaaacaaaatgggagtatatattataactgatttttccttcctcttcctctgtcattcactatccatatatttccggctatagaaagctaagaataactgtgttccacactcttgttcataggttcataaagtttttcctcctcttattcattatcaagaaacattttctatcttttaaaaaataataattagaataaaactagaaagcaaccgaatatgcctctttcccaaaagcaatttgttactttatccctacaactacccactcccccttgaaaatatattttccaactaattctttgcaataaattaggcactggatgtaagcttcttctagtgtttatttttacgcttttttctgtcattgatttcttttatatattcccatttctttctccaagatatttaaatacaagctatccgtttcagtcttgaattgtatgactattatttatcatttataaggctcatttgattgtcccaattacctatgtacattatagaacatatggaaagtaaataaatatcatcacttggaagtaactgctgccattcacattttgttttctgttcagagtatttctgcacatactcttattgttgtactttttatctataattataaattccactttagacttaacataagtattttctcattttgttaagtcttcatatatgcattatcataatttacttattcattccccagtaggttgataatttagattgtttttaatctttaatgatttaaatagtaccttgttgaaagttttgtacaaaaaaaaagagagagggacagaaaataaggctttaattatttcctgagaataaattcccacaatgaagtattttactcccctagagtaatataactactcaaactcccaatatcagtgtatgaacacccatgccaccgttccctcactgtcaccaaacattctgtttttgaatatttgatatttaataggtgaaaactgtatttaattttaatttttatgacttttataaccttaattgttatgacttttatgtattttattacatttgaacatgttcttaaatgctcattggccatttgtatttcttcctttatgtactgttgcttcatctcacttgttcagtaggtttgctttttgtataatgaatgagtgctttcttagactacagaacattctcccctcaattcccttttcttattaccagtagtattcttaagtcaatatcaggctactctatttcacttacttgccagtgttccaatcttctatccctactggtctgtcccatacttactatgctatgagctctgatttatcccctaaatctctcactaactcatccagttttctttttatccctttcctccaattcttgacagagttagagtgacttttttaatgagtaactccaaactgtcttgcagtatacatcctctaactctttgctacatttaaataagaaaattattgcctttaacatcataaaggtagtatgtatagtataaggtaccaagtatttattatctcagtgaaccaaatttcgccactcactaagcataaggtcttgggtaaaatacttaacctttttgagatttgggggtgtttgtcttcataagataataataataaagtcaattttgaattatttagggaattaaatattatcatacctaaagagtacaattttttttacattttaaatcccagatataattatactaatcagttgaattttgtatttctttttttagccatccattttctattttaacattgaaaaaaatgtacaaaaggacacagttttaaccagtttgatttttcttttctatactttggaggtcaagacatctttatgacagaagaacagaagaaatactataatgcaatgaaaaaattaggatcgaaaaaaccgcaaaagcctatacctcgaccaggagtaagaagtatcaaatgatatgggggaaaaatacaaaaacaaaaacttgcatgcttgtctcacaaaaaagaaagtagctaacatttccagtagaaaaaatttctttcacttgtagagcatcatctaaaagtgatgtttgtatcttttatgataatggtaacaacaaaaaaaattaaagaccaaaaaagaacaagaagaaatagaagatacacccttgacttcaagagatttagaagcaccccattacaatccatattaattggccaatggacacatagacaatgtgatgtgcacaagagaaagttaatcacacaactcaagaatggttgccaagtaatatcagggaaagaagtaaaccttgggatggggctgtaagaaatgcaatttcctaatattttaagaaatgaagtgttgagtgaaataacaccttacttctattttatttttcactagagatactaagtgttttaaaggaggaaagaattccagtatctactattcatatgtatggctatagagttaggtattactaatgtctgcagaagtcagggtcagtaattacagtttatgatgataaataacaatactcatgcttccatgatgaaataaacaaattgctatgaaaatgtacagctattatcccacactataaatatttttatgatgatgatctgagaggtttcagtaattcctagggtgcatcaaggaaaaaactactagcaaatattcatattgtgatgattgaatattgccttcctgagaatgatgagaaatatatactttctccattaaaatttagcactaagagtagaaatgaaacaacaatttaggaaaaatataagttggaaaattccctcagaattcctcagtggcccaaagaaataagcctgcagatggccttcagtgactagcagtggcagaaatgggaagcagaatcacagatcaacgacaaagaacgtgaagttttttttgacataaacatataggaaatgtcattctggtctcattatccttgtgcagtttcaggattctgtgtctgacaatgttagccaaggctatttggtagaagaatatgagtattgccatatgtggtatttaaatgcataatgttatagatgtgtcttggaaattccaaattctccttaacaattatttgagaatacaacaagtgaaatattaatagtactatattttaaaatggatgtatggtccagctgatcattggaaagtcttgaaaaaaaaattaaatgggtttcttaccttttcataatcttaaaatgttgatatgtgttgcatacctctggaagaagtatacagtgttgaatgtttcttttgtttgtttgtttgtttgtttgtttgtttttatcagaaaccttctttggataagggattgttattctgagaaatacacattagtaaatggcaatttaaataactaaattttaacaattaatatgctataaatcattcttacaaaaatcagggccaatgactactttgcaagaaactagaaagtcaattaatgcagaaagtacttaatgctaatgcacatgagaaaactcctttgttgttaaaagcatttctatttctctacagaacaaatttcaaggaatggtctttgacttcgtaaccagacaagtttttgacataagcatcatgattctcatctgtcttaacatggtcacaatgatggtggaaacagatgaccagagtgaatatgtgactaccattttgtcacgcatcaatctggtgttcattgtgctatttactggagagtgtgtactgaaactcatctctctacgccattattattttaccattggatggaatatttttgattttgtggttgtcattctctccattgtaggtaagaaatatttaaagttcttaaattcagttaaataaaagtgaaagctgaaacaatcaagattagattcaagatcatcccagcaatcagagataatcactgtaaatattttgatacattttatccaagtgtgtttgtttttttaacaaaatgtctcctactatatgtcagtattgtttcttattctatccatttagaacaatatacttagtgtaaatgtagtaaaacctcattcatttgaactagaactgtgctattaactaaaataaaaatgtttttgacatgttgagattgaagaaccaatatagtatctagatggttctgttaacaagactagaaatgtagtctggtactcacgaaaattgctttagagaatagacatggagtcaccagcatatagatattagtggaaagtggtttcaagataaggaaaagtattgcataaaaagagaataaagacaaggactaaacattagcaaatgcatatagtaggttctctgcaagtgtcatgtagagattttctagactaccttaaaaatgggtttatcataatgatgcacaagggaaataaggtggcatgggacatcatcagtactatacttcaaagcctggaacacataactagcctcatgagcaatagctcatttcattagacaaaactaaagctctagtaaagtaaaaagcatctgtctgtgggcctattcattcttccctaaacaggatgatatgattgggtcaatattttactatccaaaacgaggcacctgtgttggacagattttcatgccactcccctctctgaagaaactggccttatatccagctcattgtatatagcctttgattcccactaattataatccttggttataactagtgtggctaaggtataaaagttgtattctcacacgtctccttctctctctctctctctcacacacacacacacacacacacacacacactcttacacttcgtcttttcagaatgaggcatttaaacttgaaggcatttagtttcttgagctcttctttttaactttacaagtattgtttttatctaaaaagcaaatggagagagaagctgaaaataagaagactgagattacagtgttaggtacaggagaccaaaaagaattgaaatacaacattaagtacaggatactaaaaagagcatcaacctgggatcctttcagaaatgcaccagaacatacataaatgagtatataaaaatattagagtggaatttaagtaagtaccaagatcatggggagatgaaagtagcatcaaaagctaccaatactttcagatggaaaaggagtaaatagagaatttagaatatggcaatttcagaaaaagtcactgtatacatataaacttgtacctatactgttgggctgctactgttcggccaaatgtgcatttttaacttattttctttttaaattctgacttgggactttacataaactaaaagtagtttataaacatctcttaaagataaaaaaaagtactataatccttataattgaaattattgctgaatttcaaaattaagatttataatatattatgtacaattatatatatattatatatagttgtcatatttaatataactgggttcaggactctgaaccttaccttggagctttagaagaaacatatgtttattttaacgcatgatttcttcactggttggtattctcattgtttattcataggtatgtttcttgccgagctgatagaaaagtatttcgtgtcccctaccctgttccgagtgatccgtcttgctaggattggccgaatcctacgtctgatcaaaggagcaaaggggatccgcacgctgctctttgctttgatgatgtcccttcctgcgttgtttaacatcggcctcctactcttcctagtcatgttcatctacgccatctttgggatgtccaactttgcctatgttaagagggaagttgggatcgatgacatgttcaactttgagacctttggcaacagcatgatctgcctattccaaattacaacctctgctggctgggatggattgctagcacccattctcaacagtaagccacccgactgtgaccctaataaagttaaccctggaagctcagttaagggagactgtgggaacccatctgttggaattttcttttttgtcagttacatcatcatatccttcctggttgtggtgaacatgtacatcgcggtcatcctggagaacttcagtgttgctactgaagaaagtgcagagcctctgagtgaggatgactttgagatgttctatgaggtttgggagaagtttgatcccgatgcaactcagttcatggaatttgaaaaattatctcagtttgcagctgcgcttgaaccgcctctcaatctgccacaaccaaacaaactccagctcattgccatggatttgcccatggtgagtggtgaccggatccactgtcttgatatcttatttgcttttacaaagcgggttctaggagagagtggagagatggatgctctacgaatacagatggaagagcgattcatggcttccaatccttccaaggtctcctatcagccaatcactactactttaaaacgaaaacaagaggaagtatctgctgtcattattcagcgtgcttacagacgccaccttttaaagcgaactgtaaaacaagcttcctttacgtacaataaaaacaaaatcaaaggtggggctaatcttcttataaaagaagacatgataattgacagaataaatgaaaactctattacagaaaaaactgatctgaccatgtccactgcagcttgtccaccttcctatgaccgggtgacaaagccaattgtggaaaaacatgagcaagaaggcaaagatgaaaaagccaaagggaaataaatgaaaataaataaaaataattgggtgacaaattgtttacagcctgtgaaggtgatgtatttttatcaacaggactcctttaggaggtcaatgccaaactgactgtttttacacaaatctccttaaggtcagtgcctacaataagacagtgaccccttgtcagcaaactgtgactctgtgtaaaggggagatgaccttgacaggaggttactgttctcactaccagctgacactgctgaagataagatgcacaatggctagtcagactgtagggaccagtttcaaggggtgcaaacctgtgattttggggttgtttaacatgaaacactttagtgtagtaattgtatccactgtttgcatttcaactgccacatttgtcacatttttatggaatctgttagtggattcatctttttgttaatccatgtgtttattatatgtgactatttttgtaaacgaagtttctgttgagaaataggctaaggacctctataacaggtatgccacctggggggtatggcaaccacatggccctcccagctacacaaagtcgtggtttgcatgagggcatgctgcacttagagatcatgcatgagaaaaagtcacaagaaaaacaaattcttaaatttcaccatatttctgggaggggtaattgggtgataagtggaggtgctttgttgatcttgttttgcgaaatccagcccctagaccaagtagattatttgtgggtaggccagtaaatcttagcaggtgcaaacttcattcaaatgtttggagtcataaatgttatgtttctttttgttgtattaaaaaaaaaacctgaatagtgaatattgcccctcaccctccaccgccagaagactgaattgaccaaaattactctttataaatttctgctttttcctgcactttgtttagccatcttcggctctcagcaaggttgacactgtatatgttaatgaaatgctatttattatgtaaatagtcattttaccctgtggtgcacgtttgagcaaacaaataatgacctaagcacagtatttattgcatcaaatatgtaccacaagaaatgtagagtgcaagctttacacaggtaataaaatgtattctgtaccatttatagatagtttggatgctatcaatgcatgtttatattaccatgctgctgtatctggtttctctcactgctcagaatctcatttatgagaaaccatatgtcagtggtaaagtcaaggaaattgttcaacagatctcatttatttaagtcattaagcaatagtttgcagcactttaacagctttttggttatttttacattttaagtggataacatatggtatatagccagactgtacagacatgtttaaaaaaacacactgcttaacctattaaatatgtgtttagaattttataagcaaatataaatactgtaaaaagtcactttattttatttttcagcattatgtacataaatatgaagaggaaattatcttcaggttgatatcacaatcacttttcttactttctgtccatagtactttttcatgaaagaaatttgctaaataagacatgaaaacaagactgggtagttgtagatttctgctttttaaattacatttgctaattttagattatttcacaattttaaggagcaaaataggttcacgattcatatccaaattatgctttgcaattggaaaagggtttaaaattttatttatatttctggtagtacctgcactaactgaattgaaggtagtgcttatgttatttttgttctttttttctgacttcggtttatgttttcatttctttggagtaatgctgctctagattgttctaaatagaatgtgggcttcataatttttttttccacaaaaacagagtagtcaacttatatagtcaattacatcaggacattttgtgtttcttacagaagcaaaccataggctcctcttttccttaaaactacttagataaactgtattcgtgaactgcatgctggaaaatgctactattatgctaaataatgctaaccaacatttaaaatgtgcaaaactaataaagattacattttttattttattgtttgcccagtcactttttgttaacagaatattctaatgatatggagattttttacattacaaattgggggagaaggggagcgcgcgcgcacacacacacacacacacacacacacacacacacacacagaggcatacccacgttgacaacaaaacctagggtagatatgtcactggaggtagggggtaatgacctcccagaattacaagcagcaggtgtgttctctgttaggaggaagaactggtgtcagaggatagctagtgattctaggaggaagagaagtatggaagccagagtgatggtggatgaccccttgagctatgaaaagaaacccttaaatcatcatttaaaaatttagaattgccatgtgtgtaggatactgtgtttgctcctccagagccactctctctgcttctgcatcattctgtgtgtcccagaagggtgacttctacacattgcaaaaatgggctctcctacctttgagctcccaattggtttggccaatgagaagcaccagtgggaaagcaccagagagagaagattgacataggaatatttcttctccaattccttctttgctgggttggcactggactcattcctccccgaaaagtcatactccaatcagactgcccctcatacaactgaagctactttctctggggtcaggtaatcactcctccccttgctccttcaggtctgctgctgcattgagagtgcttttgtattccttgtagctttctcctaacattgctgacacttttgtaaatgtccccttcatgaaattcttctatatgcctcatttcagcatgccatctgtctcctgcctggctgacacaaggtgattcaacagctcatgaaagtcagcaggaagcaaagatgtgccttgcttcagcttggggtcttaatcttgctaacttttgcagataaagaaaaacagtaactgggggaaccacagtgaagtccagtgcagaattcacagatatcatggaaaggttactcgggtggtccagatagtaaaattaacagtctaaattaatctatctaaatttctgaggaacgagaagccttcccttgtcatcaggtgaagccagaagagggaatatagcctcaaccagaaaagggacagtaattaaaaggcttttcccatccttgtacaatggactgactttgcctcttcataacatcacaatcctaaagcaacacaacaattaattctgatatattagtagctgaaaaaaattcccatttccaactaaggtaggtcagaattataggataaaccctgcagactttttatactacccatccacgccattactcactgttacctttccaaatacaaagagaagaactggtaaaacataatcatataaatctccatattcattttgaaatatttggcatgatattttctgtgctaaaaagtaattattcttcaaagaatgatgaggtcatgtcagtaagacacaggaaccaactagaaggggcttcccactggccaaatctggggcaagttgagcatcaaaataaatgatagtaaaagattataattcattgaataagaatcagcaaatacatactgatgtaagtaaataaggaaaagtacaaatctgtttcttgcagttgaatgttaattaacaattgtagaagaaataacggagttagaaaaatcactatttggcaatcaccctaatgacaattgattcatacaagaatcatcaatgagtattaaaactcatgggtgaaagtttgatgaggaatagggtatttatagcatcttaaagtatctcttctctattaagtagaaaatttaaacagaagaaagtatactttggagaaatacagcagacaataccttcaaagatatcatcaattatgagaccaactgatactatgtgcctcctgataagatatactgaaagggccacattacttcttggtacacagtcaaaattttaaaaccagaatctaactacaaggaaaatcaaattgaggacactctataaaataagtggactgaactccttaaaaatgtcaatgtcatgaaagacaaagaaaggctaaagaattccatgaggtcaaagaactatgacaactaaacacaattctggatggaatatcaaattaaaaaataacagataaataatattattgggaaagttgaataaatttgaatatggactgtttattagttattagtattataatagtgttaattttcctaattttgttaagactagtgtgcctgttccatgaaaatagaaaatgttcttattctctgaaaatgcatgctaaagtatttaggggtgaatgcaacaatgtctgcagctcattcttgaatcagttcaaagaaaaatgagttacatttatatatatatgtatgtatgtaaacagacatagataaaagtatagatgtgtgtgtgtctttagaaaggggaggattttttttttttttttgctgtgtgttactgaagtgcctatgtctgcgtgttcacactatcatattttgtatgccctggactttataatttctaccttcaaaattagatctactgttggtaattaattcaatatatactggttttttaactactattctcatttcctagcagtaatcttcctgaaaagtcacagaaatgattacattccttgttcttcataataatcactgtttaattaaaataagaatattttagaaaagatctgcggcatagtggttaagaccccagtatttgatgctaaacagatctgatttggataacagaaggtggcactttgctgtttaagctggggaccagacactgtgggtataaatagtaattccaaacacagctccacagagcagcacccttatgacaaggttttcatatgtctatagttaagccagaaaattaagaataatgccataaatatttataaagctgaacatatccaagttaaagacctttatcctgaaattgtatcttttagattattttctaaagactaataccatttaatgtttaaatgttctttggaaatgatggtgagaatacgtgataatgggtcattggttttaatattttatttagccaagtggaaaattggcaacctggtgtcggtcctcccatttgtattttactggtgcatgaaatccaaaagtctagtaaccattgggacagacaactctactgcataagtttgtatgtttgtatatctgtatcacaaagcccagacactcgaactatataaacttgtcgcactaaagacagcaaatatgtctggtaattgcatattcttcatgtgtgcactggaatttcttattatataagaaaataaatgtgtttctaaaccaccatgaattgggtactgctgatagtactattcttcctggcacatggaaatatcccactgaggttgtcaatcaatattgatttaatcagtgtggcagcaaaggcactagaggaggaaaatactctagacaatcaagtatagatgggaaaattgcatcaccaagaaattcttaaaatcacaagtggataatttgcaatatcaattgattttactcaactgcttgagtcctttatcattccgttagaacactcctcatatttgcagataactcagcccctcacctcagcaacaagcaagaggacatcattataaacttcctcaatttctttcctatccagtgaaaaatatctaagtcccttttcccttcccttttccttcccttcccttcctttttctttccttccattctcttcctttttccttcctttttctttccttccattctcttcctttttccttcctttttcttactttacctccccctcctacctttccctttctcccttcctccctctctcccttccttcctctctccctctccttccttccttccttcaatccatacttttatatcttttcagagatcaatttttcccattcatttctacattctcagataccttgctccatgacttgttctacttctcttctatctttattctttccctcttgactgatcctttttgtctgcctttgcattttcaaaatcagtttttctaatcttaaaaaataactttgctcaaccctgatgcatctttattcactgtgtcattatctcattatttttaaaggttagtttctaaagccagggctctatactcactaccttcactcttggttaaaattaaaagaaatataggtcccagtgatatttatgggaaaatttgactgtgaataatcttgttgatataaacactttgggttttcaaaatgttatgctagctgttaactttcccagtaacattatgagggaagggctggaaaatacaaaaggtagacataggaaccgagaacaccacccctatggagccaagctatacatactctgtctctggttatcataaatacatctcctatattcaatgcttgcttagaaaggcatgatgtccacggagcaattcaattgttcactgtgatgggtttttttgttttctcctcatggaatgcaacttttatttatgagaatatggagaccagaaagtccctaaaataaatttgtaaaaaaaaaaatttaaggataaattcatcatgaaaaaagtattgttatagaaagtttaatttttcccctaagttgttatgaactgactacccctgtgttaattaaacttgccattatgcatgtgtatattttgttggaaaccattgagatacaagtcttatttttcttcttcaagctgtttgaaataaattgtgactcaaacaaattcaaataccagcccaatgctgaatcttattcttccagagcaccttctctctctccttcatggtctcaaacctggctcctacttttaccactctaaattagctaaatttgatctttattcaggcttcatcgtatctggcagatttttagaattcagtgatgatgatgatcatcacctcctccctctttaaattgcatcttactgtgtcttccataaaactagagtaccctaggcctcttagtgtatctttgattctttttcttttctttctttctttcttcttttttgtgttgttgttgttgttgagatgtagtctcactctgtcgccaggcagtggcacaacctcctctcactgcgacctccacctccctggttcaagcgattcccctgcctcagcctcctgagtagctgggactacaggttcacgccaccacacccagctaattttttgtattttttaatagaaacggggtttcaccattttggccaggatggtctcgatctcctgacctcgtgatccacccgccccgcctcccaaagtactgggattacaggcgttaagtcaccgcgcctggcctgtatctttgattctttgcctttactctctatcaatattttctctccttttagatttgtagtgcagcagcagcctttttgctgagttggtgctgcatctccacacgtctgttggacttttgtacttgcatattcagctatcaccttcaacttaatatgtccaaaactagtccctttatagtctatccctatcagccatcaacaaaactagccagaagtcaaccagctttccccatatctgttattgttcaagttatctttctaattcggcaaaagaaataagtacaattacaggatatattgaagaacaaatgagtaatgctatgtgaggatacagggaaaggttatataataggaaaacttttttttttttttctttgaaacagagtttccctctgtcacccaggctggagtgcagtggcgcaatctcgggtcactgcagcttccactccccgggttcaagcgattctcctgcctcagtctcccaagtagctgggattacaggcacccaccaccacacctggccaatttttgtgtttttggtggagacggggtttcaccatgttggccgggctggcctcgaactcctgacctcaagtaatccacccgccttggccccccaaagtgctgggattacaggcataagccaccacgccgagcctataatgggataacttctgatctgggttttgaagaataagcggggtagggcagccaggacagcaaaaatatcagaccaatggccaaaaccacaggctctagagtcagattgcctggatccaaatatcagctatactgatattacaaaagtttcttggcctctcattgcctcaatttcatcatctgtaaaaatggtaataataattcttatgaggactaaatgagttaacggaagtaaagctttcagatcaacagttccaggcatataattactacataagtctttgttttaatgatgacaattaaagacacatatgaaaatgagaaacacaaggacagttcagagcatttttaagagttcactgttgctaatgtgaaaaaggagcaatgacagtgactgagagacaagcaggactcagatgataaagggctttaaatgcagggtgggggccttggtgatgaagacccatttagaaattgtaaaagaagcaaaagtcggtgagagtagattttgacttcaagacaaaggaaacattcaatgtgaattttataattgttttgtctaattctaggaaaaatgacattggtaaaaaagcaataaatgttggtgaggtacagtgaaaaggaaaggcttatacactgttggtgggaaagtaaattagcacaatttatgtactatttatgtaccatttatgtttcctctttggaaaacagtatggagatttctcaaagaactaaaagagatctaacattctatctagcaatccccctactgggtatctacttaaaggaaaataagttattatatcaaaaagacacctgtatttgtaggtttatcacagcccagttcacaattgcaaagatatggaatctacataagtgtccatcaactgaagagtggataaagaaaatcaggggtgtccaaggtagacaatacagtcatgggttcttagtttctatttctgtttgggtcagtaaagccccttgctcatctcccttttctgcttatcactagggacagaaactaaaagtcatggcttcaggctgctaaagtctaaaacaaaacaaaacagaacaacaagaacaataaagttaggcgggacaagcttgaagactgaacagtagttatttatggcagaatactactcagccataaaaat

SEQ ID NO: 2 - DNA, 8612 bp, Homo sapiens
  source 1..8612: mol_type = genomic DNA
ttgctgatttgtattaggtaccatagagtgaggcgaggatgaagccgagaggatactgcagaggtctctggtgcatgtgtgtatgtgtgcgtttgtgtgtgtttgtgtgtctgtgtgttctgccccagtgagactgcagcccttgtaaatactttgacaccttttgcaagaaggaatctgaacaattgcaactgaaggcacattgttatcatctcgtctttgggtgatgctgttcctcactgcagatggataattttccttttaatcagaacagcataagaattatttctgagtggaggtgaggcttgtccaaatgtctttgctatcatggatttcctgactcctacctgtttgaggtttgggcaattatgaataaggctgctgtatacatccgtgtgcaggattttgtgtggacataagttttcaactcctttggttaaatcctaaggaatttcatatgcagaataaatggtaattaaaatgtgcaggatgacaagatggagcaaacagtgcttgtaccaccaggacctgacagcttcaacttcttcaccagagaatctcttgcggctattgaaagacgcattgcagaagaaaaggcaaagaatcccaaaccagacaaaaaagatgacgacgaaaatggcccaaagccaaatagtgacttggaagctggaaagaaccttccatttatttatggagacattcctccagagatggtgtcagagcccctggaggacctggacccctactatatcaataagaaaacttttatagtattgaataaagggaaggccatcttccggttcagtgccacctctgccctgtacattttaactcccttcaatcctcttaggaaaatagctattaagattttggtacattcattattcagcatgctaattatgtgcactattttgacaaactgtgtgtttatgacaatgagtaaccctcctgattggacaaagaatgtagaatacaccttcacaggaatatatacttttgaatcacttataaaaattattgcaaggggattctgtttagaagattttactttccttcgggatccatggaactggctcgatttcactgtcattacatttgcgtacgtcacagagtttgtggacctgggcaatgtctcggcattgagaacattcagagttctccgagcattgaagacgatttcagtcattccaggcctgaaaaccattgtgggagccctgatccagtctgtgaagaagctctcagatgtaatgatcctgactgtgttctgtctgagcgtatttgctctaattgggctgcagctgttcatgggcaacctgaggaataaatgtatacaatggcctcccaccaatgcttccttggaggaacatagtatagaaaagaatataactgtgaattataatggtacacttataaatgaaactgtctttgagtttgactggaagtcatatattcaagattcaagatatcattatttcctggagggttttttagatgcactactatgtggaaatagctctgatgcaggccaatgtccagagggatatatgtgtgtgaaagctggtagaaatcccaattatggctacacaagctttgataccttcagttgggcttttttgtccttgtttcgactaatgactcaggacttctgggaaaatctttatcaactgacattacgtgctgctgggaaaacgtacatgatattttttgtattggtcattttcttgggctcattctacctaataaatttgatcctggctgtggtggccatggcctacgaggaacagaatcaggccaccttggaagaagcagaacagaaagaggccgaatttcagcagatgattgaacagcttaaaaagcaacaggaggcagctcagcaggcagcaacggcaactgcctcagaacattccagagagcccagtgcagcaggcaggctctcagacagctcatctgaagcctctaagttgagttccaagagtgctaaggaaagaagaaatcggaggaagaaaagaaaacagaaagagcagtctggtggggaagagaaagatgaggatgaattccaaaaatctgaatctgaggacagcatcaggaggaaaggttttcgcttctccattgaagggaaccgattgacatatgaaaagaggtactcctccccacaccagtctttgttgagcatccgtggctccctattttcaccaaggcgaaatagcagaacaagccttttcagctttagagggcgagcaaaggatgtgggatctgagaacgacttcgcagatgatgagcacagcacctttgaggataacgagagccgtagagattccttgtttgtgccccgacgacacggagagagacgcaacagcaacctgagtcagaccagtaggtcatcccggatgctggcagtgtttccagcgaatgggaagatgcacagcactgtggattgcaatggtgtggtttccttggttggtggaccttcagttcctacatcgcctgttggacagcttctgccagaggtgataatagataagccagctactgatgacaatggaacaaccactgaaactgaaatgagaaagagaaggtcaagttctttccacgtttccatggactttctagaagatccttcccaaaggcaacgagcaatgagtatagccagcattctaacaaatacagtagaagaacttgaagaatccaggcagaaatgcccaccctgttggtataaattttccaacatattcttaatctgggactgttctccatattggttaaaagtgaaacatgttgtcaacctggttgtgatggacccatttgttgacctggccatcaccatctgtattgtcttaaatactcttttcatggccatggagcactatccaatgacggaccatttcaataatgtgcttacagtaggaaacttggttttcactgggatctttacagcagaaatgtttctgaaaattattgccatggatccttactattatttccaagaaggctggaatatctttgacggttttattgtgacgcttagcctggtagaacttggactcgccaatgtggaaggattatctgttctccgttcatttcgattgctgcgagttttcaagttggcaaaatcttggccaacgttaaatatgctaataaagatcatcggcaattccgtgggggctctgggaaatttaaccctcgtcttggccatcatcgtcttcatttttgccgtggtcggcatgcagctctttggtaaaagctacaaagattgtgtctgcaagatcgccagtgattgtcaactcccacgctggcacatgaatgacttcttccactccttcctgattgtgttccgcgtgctgtgtggggagtggatagagaccatgtgggactgtatggaggttgctggtcaagccatgtgccttactgtcttcatgatggtcatggtgattggaaacctagtggtcctgaatctctttctggccttgcttctgagctcatttagtgcagacaaccttgcagccactgatgatgataatgaaatgaataatctccaaattgctgtggataggatgcacaaaggagtagcttatgtgaaaagaaaaatatatgaatttattcaacagtccttcattaggaaacaaaagattttagatgaaattaaaccacttgatgatctaaacaacaagaaagacagttgtatgtccaatcatacagcagaaattgggaaagatcttgactatcttaaagatgtaaatggaactacaagtggtataggaactggcagcagtgttgaaaaatacattattgatgaaagtgattacatgtcattcataaacaaccccagtcttactgtgactgtaccaattgctgtaggagaatctgactttgaaaatttaaacacggaagactttagtagtgaatcggatctggaagaaagcaaagagaaactgaatgaaagcagtagctcatcagaaggtagcactgtggacatcggcgcacctgtagaagaacagcccgtagtggaacctgaagaaactcttgaaccagaagcttgtttcactgaaggctgtgtacaaagattcaagtgttgtcaaatcaatgtggaagaaggcagaggaaaacaatggtggaacctgagaaggacgtgtttccgaatagttgaacataactggtttgagaccttcattgttttcatgattctccttagtagtggtgctctggcatttgaagatatatatattgatcagcgaaagacgattaagacgatgttggaatatgctgacaaggttttcacttacattttcattctggaaatgcttctaaaatgggtggcatatggctatcaaacatatttcaccaatgcctggtgttggctggacttcttaattgttgatgtttcattggtcagtttaacagcaaatgccttgggttactcagaacttggagccatcaaatctctcaggacactaagagctctgagacctctaagagccttatctcgatttgaagggatgagggtggttgtgaatgcccttttaggagcaattccatccatcatgaatgtgcttctggtttgtcttatattctggctaattttcagcatcatgggcgtaaatttgtttgctggcaaattctaccactgtattaacaccacaactggtgacaggtttgacatcgaagacgtgaataatcatactgattgcctaaaactaatagaaagaaatgagactgctcgatggaaaaatgtgaaagtaaactttgataatgtaggatttgggtatctctctttgcttcaagttgccacattcaaaggatggatggatataatgtatgcagcagttgattccagaaatgtggaactccagcctaagtatgaagaaagtctgtacatgtatctttactttgttattttcatcatctttgggtccttcttcaccttgaacctgtttattggtgtcatcatagataatttcaaccagcagaaaaagaagtttggaggtcaagacatctttatgacagaagaacagaagaaatactataatgcaatgaaaaaattaggatcgaaaaaaccgcaaaagcctatacctcgaccaggaaacaaatttcaaggaatggtctttgacttcgtaaccagacaagtttttgacataagcatcatgattctcatctgtcttaacatggtcacaatgatggtggaaacagatgaccagagtgaatatgtgactaccattttgtcacgcatcaatctggtgttcattgtgctatttactggagagtgtgtactgaaactcatctctctacgccattattattttaccattggatggaatatttttgattttgtggttgtcattctctccattgtaggtatgtttcttgccgagctgatagaaaagtatttcgtgtcccctaccctgttccgagtgatccgtcttgctaggattggccgaatcctacgtctgatcaaaggagcaaaggggatccgcacgctgctctttgctttgatgatgtcccttcctgcgttgtttaacatcggcctcctactcttcctagtcatgttcatctacgccatctttgggatgtccaactttgcctatgttaagagggaagttgggatcgatgacatgttcaactttgagacctttggcaacagcatgatctgcctattccaaattacaacctctgctggctgggatggattgctagcacccattctcaacagtaagccacccgactgtgaccctaataaagttaaccctggaagctcagttaagggagactgtgggaacccatctgttggaattttcttttttgtcagttacatcatcatatccttcctggttgtggtgaacatgtacatcgcggtcatcctggagaacttcagtgttgctactgaagaaagtgcagagcctctgagtgaggatgactttgagatgttctatgaggtttgggagaagtttgatcccgatgcaactcagttcatggaatttgaaaaattatctcagtttgcagctgcgcttgaaccgcctctcaatctgccacaaccaaacaaactccagctcattgccatggatttgcccatggtgagtggtgaccggatccactgtcttgatatcttatttgcttttacaaagcgggttctaggagagagtggagagatggatgctctacgaatacagatggaagagcgattcatggcttccaatccttccaaggtctcctatcagccaatcactactactttaaaacgaaaacaagaggaagtatctgctgtcattattcagcgtgcttacagacgccaccttttaaagcgaactgtaaaacaagcttcctttacgtacaataaaaacaaaatcaaaggtggggctaatcttcttataaaagaagacatgataattgacagaataaatgaaaactctattacagaaaaaactgatctgaccatgtccactgcagcttgtccaccttcctatgaccgggtgacaaagccaattgtggaaaaacatgagcaagaaggcaaagatgaaaaagccaaagggaaataaatgaaaataaataaaaataattgggtgacaaattgtttacagcctgtgaaggtgatgtatttttatcaacaggactcctttaggaggtcaatgccaaactgactgtttttacacaaatctccttaaggtcagtgcctacaataagacagtgaccccttgtcagcaaactgtgactctgtgtaaaggggagatgaccttgacaggaggttactgttctcactaccagctgacactgctgaagataagatgcacaatggctagtcagactgtagggaccagtttcaaggggtgcaaacctgtgattttggggttgtttaacatgaaacactttagtgtagtaattgtatccactgtttgcatttcaactgccacatttgtcacatttttatggaatctgttagtggattcatctttttgttaatccatgtgtttattatatgtgactatttttgtaaacgaagtttctgttgagaaataggctaaggacctctataacaggtatgccacctggggggtatggcaaccacatggccctcccagctacacaaagtcgtggtttgcatgagggcatgctgcacttagagatcatgcatgagaaaaagtcacaagaaaaacaaattcttaaatttcaccatatttctgggaggggtaattgggtgataagtggaggtgctttgttgatcttgttttgcgaaatccagcccctagaccaagtagattatttgtgggtaggccagtaaatcttagcaggtgcaaacttcattcaaatgtttggagtcataaatgttatgtttctttttgttgtattaaaaaaaaaacctgaatagtgaatattgcccctcaccctccaccgccagaagactgaattgaccaaaattactctttataaatttctgctttttcctgcactttgtttagccatcttcggctctcagcaaggttgacactgtatatgttaatgaaatgctatttattatgtaaatagtcattttaccctgtggtgcacgtttgagcaaacaaataatgacctaagcacagtatttattgcatcaaatatgtaccacaagaaatgtagagtgcaagctttacacaggtaataaaatgtattctgtaccatttatagatagtttggatgctatcaatgcatgtttatattaccatgctgctgtatctggtttctctcactgctcagaatctcatttatgagaaaccatatgtcagtggtaaagtcaaggaaattgttcaacagatctcatttatttaagtcattaagcaatagtttgcagcactttaacagctttttggttatttttacattttaagtggataacatatggtatatagccagactgtacagacatgtttaaaaaaacacactgcttaacctattaaatatgtgtttagaattttataagcaaatataaatactgtaaaaagtcactttattttatttttcagcattatgtacataaatatgaagaggaaattatcttcaggttgatatcacaatcacttttcttactttctgtccatagtactttttcatgaaagaaatttgctaaataagacatgaaaacaagactgggtagttgtagatttctgctttttaaattacatttgctaattttagattatttcacaattttaaggagcaaaataggttcacgattcatatccaaattatgctttgcaattggaaaagggtttaaaattttatttatatttctggtagtacctgcactaactgaattgaaggtagtgcttatgttatttttgttctttttttctgacttcggtttatgttttcatttctttggagtaatgctgctctagattgttctaaatagaatgtgggcttcataatttttttttccacaaaaacagagtagtcaacttatatagtcaattacatcaggacattttgtgtttcttacagaagcaaaccataggctcctcttttccttaaaactacttagataaactgtattcgtgaactgcatgctggaaaatgctactattatgctaaataatgctaaccaacatttaaaatgtgcaaaactaataaagattacattttttatttta

SEQ ID NO: 3 - DNA, 176000 bp, Homo sapiens
  source 1..176000: mol_type = genomic DNA
gcttttctaattcaagcaagcttagactggctgcccatgctcaatgggttaggattttaccagaaatacaaactataaggtaaagagaatgtataggggagtgtgctgggtaaccttgattgtcaccttgacaaggtctgtagtgtcaccaaggagacagctggctaggcatgccagtcagggattgggcaaattaaggtgagaagactcattctaaatgtggctgttttcatgggctggggtcggtgctgaagaaaaagaagacatttagcagagtaggagcatttataactctctgtgttctgaccttggacatcacatgacctaagcttatgttaccatgcatctttatcaggaaacactgtatcccttctgagtataaacagaaattaactcctcaagtaccatcttgccagttgctttgtcacagcaaaccaaaaaaacaaacaaacaaaaaaaaaaaccagagttattctggacactcctgtagaaagtggttttggtttggtttggtttggtttggttttttgtttttttccacagttaagcatcatgagatgtaagttagtctccagatggtcctccataaatatttttagagaaacaattgaactgctggtacctgacatctattctttcccatttctcttatgtacattgcagtcaagctgccagtggagagagaggcagccacgctgaggccatgagtcaacaaacaagaggataaaagctcagaacacagctcagcaggacagggatggctgaatatgtgtgcagctgagtttggcactaagcatcattaaaaccaaaccagaacagacaagaggatgaaagtcataggctgaaaatggtcaagaggaagaaaactgagctcaaaccactaggcatttgctggactggctctgaagtcctacctccagatgactacatcactgtggaaattccatattcccttcattaagctggtgaataccatattatatctgatgtggccaaactcgtactgccttgatccaataggagaatctttacattaaacattataaaaatggtttgtgaacaagtatttcagaagcatccatttgcatagaaaattactaaattaacatgttaataaataattttgtagacaatttaagtcactctccaaagaactcaactaaaccatagtaggtgcagctccccacacatgaatacattgtgaaccctgttcattccactattcattcactcagcaaatattcaatgatgttcagtaagtactgtcctaacagcagagacacaagagtgtgaaaatcagtttcagactgcaagagactcgtcgtttttcaaaaaaaaaaaaaaatgtaagacagaactttttaaaaatgatatgcatattccagatccttgccccctgtgtggttgattcccaggtcattaatgcttcaaaacagggagaagcctattgcataattcacatcagcaccatttctatcactcaatattcaaaatataactgaaggctttgaaaatgtaactcaactctaccccaatatgttctttacagttctggggaggatggagagggaggagatttctcattctatttactacagaacttggtttttttccaaaattatactactgaaaaaaaaaaaaagcaaatgtttagtgacattataatctgtctggagaaaaaaaaaatgactgtaaatccagccgatggtctagtaaaaaaaaaaaacaaaaaaaaaaaaaaaacaaaaagacaaaaaaacaaaaaaacaaaaaaaacaaaaaaaaaaaaacaaatgaaaacaagtatttactatactgcagtagcaaaactaccaggtgaatttgggggaaacctcacctacgttggctcagacacatttaacttcttttaatgtctatttccttactcgttaattaacaggagaaagtagatcaccgtgttctttcatgcccctgtatcgtctcatctcctcaccctggtcttaatgtggctcatagcagcaaactccctagctaagaaaagaaggctgcgattatttttaagtagtgcatggcgtcctctctatttctgtcacctccaaaactagatggttttgtccacaacccatgtaagaccatgattttacatataactaaatagtatgatgtcatcttgtgctcatatgaaatcatcacactcagaatcatggtaggagactggatgtactgtttgtaggttttaagactgtagctcatgttacatacacttgagataattgattgttaattaggcagtttaatattttttaatctgctgcttaaaatattccccttggtatcaagaagtgaatctaagaatgaccatctttttaaaataaaataaaataaaacaaaataaaataaaatagaataaaacaatttctatagagattgttttgaaagtctaaaagaatggttcactatatgcaaaatctaaggagaaagctcagaacctcttttctcagtacagacagtacaattatcacatctgatatttatctgcaaaccacaaagatgtgtacctgctcttattacttggatgaacatcaggagttaaaatgcattaaaacagtttaacccagcacagtctcaatttgatggagaagagtaattttactatcatcttgtttattaaaatgtgtctaaagtattgagcacttcacagatttttttttttttggttattactctgcaaagcaacctacacaaccaactctcattgcatactgccacatcccctctactcaccacaatgagttaaaggcagaccaaagctctcagcagcatcctttgcatttattacaaggcttcaccagccccttccttcactccagatgtgatggttcaggagttgacagcaggagaagtcgtcccctcgggtttactgcacactccaagagaggtcccctgcacttgccaggagatttagctttcatgcttctggtcattcttcctacccacaaaatgagacctatttctgggctattcataactctccttccaaaacgtttcaaaaacgaaaagcaagacctggttcatgatttccatgcaagatctcagcctgcccgcctgcttgctgctgccaatacttcttactcttgctggtgatgacatcgtgcaagctccccagcactgatgcttcattatgtaagtctgtctaggtcaagtgtaggagactcactaccggcctgtggaaactcatggaactgtttcttcagattaacacttcaggggctatcgaggctgcaggaagctgagctttaactacatacatctttcgggggtaaatatttttttgcatgattttatgttttgggttttgtttttttttaatcctttggcgggctcctgtcagccaggctgataggtgcaggtaggcaattctaagaggttccaattctgtcagcatgcttgaataagcatggaaagtctcgcactggttctagaattaagacacgattaaaaacatcctaaattgaactttgctaatgaagccttttttatggtgttgatttgatgtttaaaccaagtaggattcgtttgatctgaggcgttgcaggagagcattgttagatgttgctgtgagcgtgaaaagatttactgcagttcgctgccgcgctgcccttgtctctaactgagaattcgcttgtcttgtaaatgagctctgagtctgctgcactgtgtcttccgccgcgcctgtttgtgacaagaacggagcttgaataacctggtagttaggggacttctgctgataataaatcctgcctacttagtctcgggatgcatactgcagcagctccagtgtcatttgatactagcgctgaatgaggttgggttaactgcattgggtcaggaggggctgtgcttaggagatctggcgggctgagccctggtttgatgtgacccttaactcatctaagagtaaaggtaaaaccagaggagaactggttctatcagactatctcgctttgtgtcctgctgcaagaactagttaaaacggcatactcagaacggcacactgcagaacttgcagggcaagtagcaaggtgaggttgccgcagcggtgtgagattggcaggaaattttcacacaattaaaaccgagatgatgacacaaccgagtgcacctcatttaaaatgacagagggatttaaaaagttggagatcaatgcagcatgtttgaaagtttggcttcctttgtgtccttggcaagcaagggtctgtgattgggacttgaccagagttaaaagtttcctgtcatttgcttgggtgtcttcaaatgagctctggtgtaagcagtaaggcagactaactacaggattactgtggatagaacattgaaaactgccttatattcttctcactattgaattaaaagaatgaaaaggagtgttaccgaaatcaatttgcctgggtgccattttcatactgctaagcacaatgaacaggggcgtgttgatcatttgtgattgactaaatttgtcctgtatttaattgattgaagatgtacacattttcccaaatcatttgaagagacagttacggctccctaaaggcctgattttatattactgatgtcaatagcaacgatccccgtagatacagctcggcaggcccaatagatcaaggtcactcagtctagcagaaataaaatgttaaaaataaagaatgcatacagaataatatttatattatttgtttggatgtcttccaatttagggtattttatatataatatttttatataatacttattatatttagggtatcactcttctcctcaaataacatatgattattccttaatgttgtctctataggctatcaaaaacctacagatttcttcctttttagactttttgaaatttgaattatgtctaaattaccacttatatgattatagtcctttgtgtgaattaacattaatcactcttaaagtgagattacatgaagggtgagattcaagtttactcttagaccagcatttgccatttccccatcaaatgttaggcagtgtttctcagtggtctgagaaagctagctactagctatgtcagcatctgaatggaacatttggaaatgcccgtctttgggcctcagggcagacacacttagaacttccctttgtaggttctagaaatgcaagttcttacaacctccaggcagtagtgaggtgtgtgaagatccagtacatgggacagtaacttggtcagatatgaaaatggttttcatacaatatctgaataggtgttacataattaaattctctgtttcttcttgcctggcctaactatggctagctgtggactgatgtttactgtgcagttatgtaccatatctgaagtgcaaggggttcccagcaatattggtttttcagcagttccatgtggaatactagctctttgaagctaaatggcacgatgtaaatacactagaggacctacctctcttatatgctcagagcgactttgcacatatcactggcccctttggtgctcagtttctctctgtaaagtaagtgattgttctaacacaagattgcggctgggcctaattgggtgtcttagtcaggctttgtgttcctgcacaaaacatgatcaagaagcaagttagggaggaaagggtttattgagctaacacgtccacattgctgttcatcaccaaaggaagctggaacaggaactcacacaggacaggaacttagaggcaggagctgatgcagaggccatggaggagtgctccttactggattgctttgtgtggcttgctcagcttgctttcttatagaacccaggactaccagtccaggcatggcaccacccacattgagaaatgccttacggctagatctcatggaggcatttcctcaagggacgcttctttctctgtgataactccagctgtgtcaagttgacatacaaaaccagctagtatttttggggtttggggtcaggaattcttaggggtctctcttgtgcatagcagaattctgaccactatcactaacctgttgaactgatgtgctggtagagactaacaaatgtccccccaaggcagaactgttcccacacagcaatttaagtacatccaatttagattatctttcttatgaacaattacattatagtaaacaagacaacaaatgtgttgcttctcctactggtaagttaaattacaatagaaataaatatatggccacgtatgtaatatagtaaatttagaccccatcatattcatgaaagtgcatgtgtaattcagttaagtacccacagactgtggaaagccgaactctttctgcagcttcccagggacctagctcttcaccatcctgtttgtcctgaagtctccagatggcccagacaccctccactactagcctataccaaatatttagcaaagtgaaagacttttagattagattgaatccacttaactgaagatagggatttatattctcattttttttttctgcacctcttatccctcccctctttgccatccctgcaacatccaggagtgccccagtttcctcgtgtgacatgtacaatagtctgtctttccgtagctttttatccagatgtagacacacaaactcattaatgaacacaggcaatcaaggaccccagtgtaggactatgcacccagaaggaaatagatggaatatattatgtcatagagctggaaagagtgttatattcacagtcgtctttgcatccaagttatactaactctcatggcctttcaaattatacgtagtctatgttagagcttcagaaagtcaagtaatctcttcccaggaattattacctcaaaaaacttagcaacacagtccagatattttctagcgtgtatatgtatggtagaaatatacaaactttacaaaaacacaaatgtatacatgcatgcatacatatacataagcacacctactcataaacacatacatgcacatatacacaagcacacaaacacacagacaagcacacacacaaatacatacatacataaacacacacacacacactttttctacttctaagagcttttttgatatcctgttctacaagtacaagctgagatctgagtgaactcagaaatctggtctaagatgtatgtaactctatttctattaggcaaaattattacctgataattgcaatagtttatccacatttctaagtaaattcacacattatatgttctgagacccacagaagtttaaaaagataggttcttttgatgctatcacttttaagaaaaaaataataaagttgagatcacaaggtaccaaatctatgtctctctctctcttctcttctctcctacagagcagaacacagcagtatgtacactctgacttaaagtaaaatccccacatgatccagtaatcctaggaatacagctaaagaaacagaatttgatacttcagggtggtttttgtgtggccgcttccgtgtttttcacagtagccacagtatggttccacttaggaattcaccaatgggaacttagcaggagcaagggcctgaggctcactcacattgcttctgtgtggctggggctgggattctgatcttctctaatacagtgacactatactgtttcgtgaggcttgttttgttttgttttgttttgctttgctttgctttgctttgctttgctttgctttgctttgctttgctttgctttgctttgctttgctttgcttttatatcaacaagtaggaacaaaagaaataagacatgggaaaagccatgcaaattctgttgaaactgtcctgtgtgggaacatatgagcagaacaggtgtcaattttccatatgacatttcccaaaaatagtatcatggtcttgtaatgttggaaaagaagaaagtagtaataacagtgaagggggccctggtttccagcccccatagtctagtgagattgttagaagtagatttgtttgttttgggttttttgttgttgttgttgtttgttttttgttttttgtttttattttttattaggtatttagctcatttacatttccaatgctataccaaaagtcccccatacccacccacccccactcccctacccacccactctccctttttggccctggcgttcccctgtactggggcatataaaatttgcgtgtccaatgggcctctctttccagtgatggcctatctaggccatcttttgatacatatgcagctagagtcaagagctccggggtactggttagttcataatgttgttccacctatagggttgcagttccctttagctccttgggtaatttctccagctcctccattgggggccctgtgatccatccaatagctgactgtgagcatccacttatgtgtttgctaggccccggcatagtctcacaagagacagctacctctgggtcctttcgataaaatcttgctagtgtatgcaatggtgtcagcgtttggatgctgattatggggtggatccctggatatggcagtctctacatggtccatcctttcatctcagctccaaactttgtctttgtaactccttccaagggtgttttgttcccacttctaaggaggggcatagtgtccacacttcagtcttcattcttcttgagtttcatgtgtttaggaaattgtatcttgtatcttgggtatactaggttttgggctaatatccacttatcagtgagtacatattgtgtgagttcctttgtgaatgtgttacctcactcaggatgatgccctccaggtccatccatttggctaggaatttcataaattcattctttttaatagctgagtaacactccattgtgtagatgtaccacattttctgtatccattcctctgttgaggggcatctgggttctttccagcttctggctattataaatagggctgctatgaacatagtggagcatgtgtccttcttaccagttggggcatcttctggatatatgcccaggagaggtattgctggatcctccggtagtactatgtccagttttctgaggaaccgccagactgatttccagagtggttgtacaagcctgcaatcccaccaacaatggaggagtgttcctctttctccacatccacgccagcatctgctgtcacctgaatttttgatcttagccattctgactggtgtgaggtggaatctcagggttgttttgatttgcatttccctgatgattaaggatgttgaacattttttcaggtgcttctctgccattcggtattcctcaggtgagaattctttgttcagttctgagccccattttttaatggggttatttgattttctgaagtccaccttcttgagttctttatatatgttggatattagtcccctatctgatttaggataggtaaagatcctttcccaatctgttggtggtctttttgtcttattgacggtgtcttttgccttgcagaaactttggagtttcattaggtcccatttgtcaattctcgatcttacagcacaagccattgctgttctgttcaggaatttttcccctgtgcccatatcttcaaggcttttccccactttctcctctataagtttcagtgtctctggttttaagtgaagttccttgatccacttagatttgaccttagtacagggagataagtatggatcaattcgcattcttctacatgataacaaccagttgtgccagcaccaattgttgatgatgctgtctttcttccactggatggttttagctcccttgtcgaagatcaagtgaccataggtgtgtgggttcatttctgagtcttcaattctattccattggtctacttgtctgtctctataccagtaccatgcagtttttatcacaattgctctgtagtaaagctttaggtcaggcatggtgattccaccagagtttcttttatccttgagaagagtttttgctatcctgggttttttgttattccagatgaatttgcaaattgctacttctaattcattgaagaattgagttggaattttgatggggattgcattgaatctgtaaattgcttttggcaagatagccatttttacaatgttgatcctgccaatccatgaacatgtgagatccttccatcttctgagatcttctttaatttctttcttcagagacttgaagtttttatcatacagttctttcacttccttagttagagtcacgccgagatattttatattatttgtgactattgagaagggtgttgtttccctaatttctttctcagcctgattattttttgtatagagaaaggccattgacttctttgagtttattttatatccagctacttcaccgaagctgtttatcaggtttaggagttctctggtggaatttttggggtcacttatatatactatcatatcatctgcaaaaagtgatattttgacttcctcttttctaatttgtatcccttgatctccttttgttgtcgaattgctctggctaatacttcaagtactatgttgaaaaggtagggagaaagtgggcagccttgtctagtccctgattttagtgggattgcttccagcttctctccatttactttgatgttggctactggtttgctgtagattgcttttatcatgtttaggtatgggccttgaattcctgatctttccagaacttttatcatgaatgggtgttggatcttgtcaaatgctttttctgcatctaacgagatgatcatgtggtttttgtctttgagtttatttatataatggattacattgatagattttcgtatattaaaccatccctgcatccctggaataaaacctacttggtcaggatggatgattgctttaatgtgttcttggattcggttagcgagaattttattgaggatttttgcatcgatattcataagagaaattggtctgaagttctctatctttgttggatctttctgtggtttaggtatcagagtaatagtggcttcataaaatgagttgggtagagtacattctacttctatcttgtgaaaaagtttgtgcagaactggaattagatcttctttgaaggtctgatagaactctgcactaaacccgtctggtcctgggctttttttggctgggagactatttataactgcttctatttctttaggggatatgggactgtttagaaggtcaacttgatcctgattcaactttggtacctggtatctgtccagaaatttgtccatttcgtccaggttttccagttttgttgagtatagccttttgtagaaggatctgatggtgttttggatttcttcatgatctgttgttatgtctcccttttcatttctgattttgttaattaggattttgtccctgtgccctctagtgagtctagctaagggtttatctatcttgttgattttctcaaagaaccaactcctcctttggttaattctttgaatagttcttcttgtttccacttggttgatttcacccctgagtttgattatttcctgccgtctactcctcttgggtgaatttgcttccttttttttctagagcttttagatgtgttgtcaagctgatagtatgtgctctctcccgtttcttcttggaggcactcagagctatgagtttccctcttagaaatgctttcattgtgtcccaaaggtttgggtacgttgtggcttcattttcattaaactctaaaaagtctttaatttctttctttattccttccttgaccaaggtatcattgagaagagtgttgttcagtttccatgtgaatgttggttttctattatttattttgttattgaaaatcagccttagtgcatggtgatctgataggatacatgggacaatttcaatatttttgaatctgttgaggcctgttttgtgacctattatgtggtcaattttggagaaggtaccatgaggtgctgagaagaaggtatatccttttgttttaggataaaatgttctgtagatatctgtcagatccatttgtttcatcacttctgttagtttcagtgtgtccctgtttagtttctgtttccatgatctgtccattggtgaaagtggtgtgttgaagtctcccactattattgtgtgaggtgcaatgtgtgctttgagctttactaaagtttctttaatgaatgtggctgcccttgtatttggagcatagatattcagtattgagagttcctcttggaggattttacctttgatgagaacgaagtgcctttccttgtcttttttgatgactttgggttggaagtcaatcttatcagatattaggatggctactccagcttgtttcttcataccatttgcttggaaaattgttttccagcctttcattctgaggtagtgtctatctttttctctgagatgtgtttcctgtaagcagcaaaatgttgggtcttgtttgtgtagccagtttgttagtctatgtctttttattagggagttgagaccattgatgttaagagatattaaggaaaagtaattattgcttcctgttatttttgttgttaaagttggcattctgttcttgtggctgtcttcttttaggtttgttgagggattaccttcctgttttttctagggcgttgttcccattcttgtattggtttttttctgttattatcctttgaagggctggattcatggagagataatgtgtgaatttggttttgtcgtggaatactttagtttctccatctatggtaattgagagtttggctgggtatagtagcctgggctggaatttgtgttctcttagtgtctgtataacatctgtccaggctcttctggctttcatagtctctggtgaaaaatctggtgtaattctgataggcttgcctttatatgttacttgacctttttcccttactgcttttagtattctatctttatttagtgcatttgttgttctgattattatgtgtcgggaggaatttcttttctggtccagtctatttggagttctgtaggcttcttgtatgttcataggcatctctttctttagatttgggaagttttcttcaataattttgttgaagatgtttgttggtcctttgagttgaaaatcttcattctcatccattcctattatccccgtaggtttggtcttctcattgtgtcctcgatttcctggatattttgagttaggatctttttgcattttccattttctttgattgttgtgccgatgttctctatggaatcttctgcacctgagattctctcttccatctcttgtattctgttgctgatgctcaaatctatggttccagatttctttcctagggtttctatctccagtgttgcctcactttgagttttctttattgtgtctacttccctttttaggtctagtatggttttgttcatttccatcacctgtttggatgttttttcctctttttctgtaaggacttctacctgtttgattgtgttttcctgtttttctttaaggacttgtaactttttagcagtgttctcctgtatttctttaagtgatttattaaagtccttcttgatgtcctctaccatcatcatgagatatgcttttaaatctaggtctaggttttcgggtgtgttggggtgtcctggactggacgaagtgggagtgctgggttctgatgatggtgagtggtcttggttcctgttagaaagattcctacgtttaccttttgccatctggtaatctctggagttagtagttagagttgactctgtttagagattgttcttctggtgattctgttaccgtctatcagcagacctgggagacagattctctcctctgagtttcagtgctcagagcactctctgctggcaagctctcttacagggaaggtgcgcagatatcttgtttttggacctcctcctggccgaaaaagaaggcccaaaacagggcctttctccgaagctgtgttgctttggcagttcccagaagctgtcagcttctgtggtgcagactctcacctgtgcagactaaattcctaagttccagggagtcccggaaccaagatggcgaccgctgctcctgaggctgaggccgcctcccaagccaagcggacacctgtcctctggtccggacggtggccggctgtctgtggcccgccaagggtgctgcctcagcggctctgtgcttccgcctatcccagaagctgtctggttctctggcgcaccctctcacctgttcggactaatttcctaagttccacggagtcctggaaccaggatggcgaccgctgctgctgaggctgaggccgcctcccaagccaggcggacacctgtcctctggtccggacggtggccggctgtctgcagcccgccaagggtgctgcctcagtggctctgtgcttccgcctatcccagaagctgtccggttctctggcgcaccctctcacctgttcagactaatttcctaagttctgctgagtcctggaactgttttttgttttttgagacagggtttctctgtgtagccctggctgtcctggaactcactttgtataccaggctggcctcgaactcagaaatctgcctccctctgcctccccagtgctgggatcaaaggtgtgcgccaccacgtccggctttagaagtagatttgtattgatccgtacaatgaggacagaaaaggtattcgtttgtccttccaacttactaatcattgttctcctagcagtcaaccctctctcacagatataacttctcaactgtgattgaagaacaatttagccaatcagaaggatgctactattatgtcactgccctgtaaggaaatagaaatctccctcttactgtaagaaaggaataatacatttctatgttgaaagacatctgggttatttcctgcttctggctattataaataaggctgctgtgaactcataggtggatattagcccaaaagctcggaatacccaagatacaattcacagatcacatgaatctcaagatgaaggaagaccacggtgtggatacttcgatccttcttagaagggggatcaaaatacccatgggaagagatacagagagaaagtgggaagcagagactgaaggaaatggcctccagtgtctgtcctacctgaggatccgtactatatacaattaccaaacccagacactattgtggatgccaacaagtacttgctgagaggagcctgatacagctgtctcctgagaggctctcccagtgcctgacaaatacagaagtggaggttcacagccatgcattggactgagcacagggtccccaatagaggagctagagaaaggacccaaggagctgactaggtttgcagccccataggaggaacaacaataagaaccaactagtacccccagaactcccagggactaaaccaccaaccaaagagtacacacggagggactcaatgtagtagaggatggccttgtgggacatcaatgagaggagaggcccttagccttgtgaagactcgatgtcccagtgtatgggaatgccaggacagggaagtaggagtggatgtgttagtaagcaggggggagggggtgagacaggatagctttcagaggggaaaggaagaaaggggataaaatttgaaatgtaagtaaagaaaatatctaatttttttaaaagaaaggaacaatgcagtaagtccttgctgttgtcttctcagcatggttttcccgatttccatgtgagcctggctccagttttctgtctttgtcagatgctctaggcccatgggatctggacctcaccactcctgccgcgctctagccgtgtgcttggatctgaccctctttctgtgtatacataaccatcacctggccctcccaaaagcctcaggtaaattgacaagcaaaatcaaggcgttgcctagatgtgccctcccagaaaacaaaactgtgggaaggcaaggttactactgctggagtattcaacagcaccacacctttgcagggaagctgattggaatctgccattgtgtaaaagcagatgtttcctcaaaacgtactttagagaaaattttgtatagtggaaaggtgcttttaggttaataacaaggaagtaaatgaggtcatttaaaccaaacaagttatgtagctatatatagcgcatgatatcattgctggaaaactgaatttcatgtacttaaacacatgcataaagtttcagaaaaggaattgtttacgtctactacctgtgtagaatctgcttaggcaaagaaaacgtggattgctgttgctctctcttttattaatttattttttctcctcatatattatatccagttcacagcttaccctgtctcctctcccctcactcctcctgaacctcccctttgcctcctcccagttcacccattcctcctccatttcccatcggaaaagggcagtcttcacagggatagcaaccaaacatgacatatcaaattgcaacaaagctaggccccttcccttgtatgaaggctggacaaggtgcctcagtggaaaagaggagggtcccaaaagcaggccaaaagagacagagacaccccttgcttccactgttacaagtcccacagaagactaagctacaaaaccataatatatatgcagagggcatcggtcagtccatgtaggctcactggctattgattcagtgtctgtgagcccctattaatccaggttagttgattctgtgggcttttctgtggcatccttgacccagctagctcctacaactccgccctccttccaagaattcctctagctccacttaatgttaggctgtgggtctctgcatctgtttgcatcagttactagatgaaaccactctcatgatgatggtgctagactcctgtctacaagtacagcagaacctaactaggaatcatttcattgacttatttttttcaatagtgtttggttctatctgaggtgtctgggatatccagcctctctctgggtcctgaccttccagtcagtatcagccctgggctctctctctatgggtttcaagctgaaccagccattggttggccactcccccaatttttgtatcaccacagtacatcttgtaggcaggaaaaattataagttgaatgtgctgaggttgagttggtatcctaatccctccactggatatctagttataggagatggctagttaaggttctgtgtcctcactttctagggatcctagctaggtcacccttgtagattgctgggagcttccattgtgttaggtttctgcatcactgccaaaatgtccttctcctctttccagttctctctctctctctctctctctctctctctctctctctctctctctctctctctctctctccctctctctctcgcttgctccatgtcctcccccttccccacatgattcttcttgtcctcctccctacccaacctagtctacccacaatatctattctactttatcttccaagggaaattcatgtgccccccacacactgagccttccttgttgcttagcctctctgagtctgattctttactttacagagaatatactcttataactgaatgtgtaccgtgtttttctctctgggtctcactatggatgacttttttctagatctgttcatttcctgcaaatttcatgataaagctggtataaacatagttgagcaactgtccttgtggactgttctgtgggtatatgctcaggattggtatagttgggtcttaaggagctttaagacagagtttctctgtctttcctggacatatctctatagaccagtctggcctcactcagagatctgcctgcctctgcctctgagtgctgggattaaaggtgtgcaccaccacaactagctgtaccccatttttaaattggattgtttgttgatgtctagattcttgagggttttttgtttgtttgtttgcttgcttgcttgcttgcttgattttacatattttagaaattagctgtctgtcaggtgtggagttagtgtatatcttttcccattctgtaccttgaatattttgtccttttgatggtgtcctttgccttacagaagcttttcagtgtcatgaggaaacatttattaaatgtccatcttaatgcctgcactattgatgttctcttcaggaagttgtcttctgtgccaatgcattcaaggctattcctttcttttctatcaggtgcaatgttttgatgttggtgtctctggttcatgtggccttgagttttgtgcagggtaatatacatggatctatttgcagtcttctacttgccaacatccagttaaaccagcaccatttgttgatgatgctttatttctccattgtataattatgtcttctttaataaaaaatcaggtgtccacaggtgtgcggatttacatctgggtctttgattcgattgcactgatcaagttgtctgtatttatgcctataccatgtgtttttatgatgatagctctgtcaaagagcttagaatcaggggtgtggtagttttaggaagttctttttttgtacaggattgttttagctatccttggttttttggttttccatttgaagttgagaattgttctttccaggtctgtaaagaaatttttttagaattttgatggggtttgcattgagtctgtagattgcttttttttactatgttaatctcacaggtccatgagcataagagaacttttaatcttctgacatcttcaatttctttcttcaaagacctaaagcttttatcatataagcttttcagtttcttagttacagttgccccaatatattttatattatctttatctaccatgaagtgtgttgcttccttgatttcttttttctcaacccatttgatgtttataaggatactgaattatttatttatttatttattatttattttagttaatcttgtatccagctgctttgctgaaggtatttatcacctgtatgagtttcctggaaaaattttatgggctgcttgtatatgctactatatcatctgaaaataataatacattgacttagtcctttccaaattgtattcctcttgatctcctttggttgtcttattactctagctagaatgtcaagtactatggacagtcttgtcttgtccctgatttttgtaaaattgcattgagtttctctctacttaacatgatgttggctatccacctactgtatattgtctttgctatgtttaggtatgtcccttatatccttgatctctctaagtcttgtatcatgaaggagtgttgtgttttgttaaaggcttttgtagtatctaatgagatgatcacgtcgggtattttttcactgtttgtatggtgaattacattgacagattttcatatgtagagccatccctgaatctctgtgatgtgtcctatgtggtcatgttgaacacatcttttatgtatattggatttagtttgtagatattttcttgagtatttttataccaatgttcatgggcaaaaattggtctgtaattctctttccttgttgagtctttttgcagattgggtatcatggtgactgtggcctcatgaaatgaatttggtgatgttccttctatttctattttgtggaataatttaaggattattgatattagctcttgtttgagggtttcctttttgtgaggtaggggagttgggagccttttcaaatgactgcttcctgtgattccttccttataggtcataggtctattttaattgtttatctgatcttgataagtggtatctattgagaaagttgtccatttcttttaggttttctaattttgtggagtaagtttttgaagtgcgacctaatgattctttgtatttcctcagtatctgttatcatgccccccttttcttttctgattttgttaagatattctctctctctctctctctctctctctctctctctctctctctctctgccttttagttggtttggataatggcttgtctgtcttgttgattttgtcttgttgattttctcaaagaactaaccctttgtttcattaattctttgtattgctctctttgtgtctattttactgatttcatccccgagtttatttcgtgccatctactcctcttgggtgtatttgcttattttattctagagcttttaggggtgctgttaagttgctagtattaaatctctctaatttctatatgaaggttcttagtgttatgaactttccgcttagcactgctttcattgtgtgccataggtttgtgtatgttgtgcattcattttcattgagttctagaaagtctttaatatcttcctttatttctgtttgacccaggggtcattcagtagagagttgttcagtttacataagtttttaagctttttgttctttctgttgttgataaaattcatctttaatctatgatggtctgatgagatgggggagttgttccagttttcttggatctgtagagacttgctttgtaacagagtctgtggtcaattttgaaaaagaagtttcatggggttctgagaagaatgtatattgtttttatttgggtgaaatgttctgtagataactgttaagtccacgttaagtctgttagtctgttaactccattatttctctgttaagtttttgtctgaatgacctgatgtctcccactgtaaatacatatttgggggggggcttcaacacagggtttctttgtatagccctggctgtcctggaactcactttgtagaccaggctggcctcgaactcagaaatctgcctgcctctgcctcccaagtgctgggattaaaggcgtgcgccaccacgcccgacttaatacatgtgttttgatgtgtggttttacaaatgtgggtgtccttgctttgaggggcatagatgtttagaattgagacatcatcttggtggggttttcctgtggtaaatatgaagtgtcctctgtctacttgattaatttttgttttgaagtcgatttgtcttatattacaacagatacaacagcttgtttcttgggtacatttgcttggaaaatctttttccaaaccctcaactcttaggtaatatctagctttgatgatgaggtgtgcttcttatatgtaacaaacggataaatcctgctttggtattcattctgttagcctgtgccttttgaggaattgagtccactgatattgagagatactaatgattaataagtgttaattcttattattttgttgttgtttatggtagtgctagtgtgtgtgtgtgtgtgtgtgtgtgtgtgtgtgtttccctctaatttttgtatgagcttatttatttcttcatgggtgtagttaacctcctagggttgttgatttccatttgtacctcgatatgcctggatttatgagtagattttgtttaaatttgactttgtcatgcagtatcttgtcttacccatctatgaggatgaacagttttgctggcgggctggagagatggctcagtgattaagagcaccgagatcctaagttcaattcccagcaaccatatggtgcctcacaaacatctgtaatgtgatctgatgccttcttctggtgaatctgaagagaccagtggtatactcatataaaaataaatctttttattttaagttttgcttgttagacgagtctaggatggtatctatggtctctcagagcctgtaagacatatgtccaggctattctggcttttagggtctctgttaagaaattaaatataattctaataattctaccttttcatgttatttggccttttttctcttgcagcttttaatatttttctttgttctgcatgttcagtgttttgattattagttgaccagggactttcttttccaattcaatccatttgtgtcttaagagatttttctgcctttagaaacatgtccttcttaaggttaggaaatttttcctctatggttttgttaaaaatattaactgagctctgtgggtgataaaacacagaaaggatgtctacagagtgtgccttggggatccaaaccaagggcataggaggtttctgagtgcagatgggcctcttactcattctcattagcctggcctgcacctgtgctctggagagtcacctgcgtagaggcaagtgcaagaagtgggagcaggcagtagggtggtggtgtcagtggcttgggtgtttgggctccaaacctagttatcaggagggttcagttatctggtggggctcttcccttttctgactggtatggcctgcacctgtgcagtaaataggaaggctaggttttacttaaagagccagcatggtgatctaattttcaaactgttacttcaagagtagattaggagatagcttctctggattggagtgtggagtgcccagaaaatgatgagaagggacaaattgtttagtcacatgctctgggtacagaaaaataggtagccttataaacgttaggtcacatgggtcatgaaatcagaagggaggtttatttaggaggagaaaggagaccaaaaagaagaggttggagaagggtagaggagtaaggggtaaacaaataagaacaatgatttgttatatacatatatagacattatatatatgtatatgtaatgacatatatgtatatatttatgcatatatggataaatatatataatgtcataataaaaacattattttgcatgcaaaattaaaatttaattaaaaataaaagctcttatgagaggtgtgaaagtattagctaatgttgtgggttgtccaccagagaagagtgcatggacatgggtttaagacaatagaaagtctttatcagctgggtagagacaacactgggtattcatgatcctgtataatcctgaacctttctcagagtgaaaccacctccggaattgtgttagtaagaacagttagccaaagcagaactacagaagccaaaaatccaaaggtggtgcatttagagatggttctagaactatgaactttgatggatcagatctttgtctttgttgtgtactgtctatgtgctgagcttttagggtctaaatgggtagttcacaatctgtgggtcatgaacccttggtggggacaaatgacccttgcacagcgattgcctaagactatcctgcatattagatatttatagtacaatccataacggtagaaaaattacagttatgatgtagaaacagaaataattttatggttggctgtccctacaacttgaggtgatatgaaaagattgcaggagcattagggaatggtacttgcgtcataacatcagttatgttaagttctcaagttcctgttatagcggcactctacagcaacacttttaaacagtgtctactggaatcacatgggcagctcgtcaaaacgtcctttctgatctggcaggttgtgggacaaaggaaatggtccaaaattttaacaagcccctcaaagatggagatgctgttgttccactgccacactcggacgagaagcaaggctttaaagcttacaaagttttatgttctcatgtgatccgtgtcttcacaaatatccagcagagccagaaagcacttgccttttcaaaaaatgtgctgagaaaaagaaaaacatgacattgaatattatatttgaaacatgaagctttgtcagaagcctcattgtcaacctctcacactcagaaattctgctcttttcatttttttatgttcaacaaatgtcgacttgtggatactaaggtactgacagactgagagattcttctcccctgaatgctcctctgaaatttagttgatgtgttcatttggcaagagaatttccaaacagtcatatcctttcacatgttgtaggatgtagggttgtgtcatcacaagtctgtcccaccactgagcacggccacttgtggagactggatgggggagtttgactccgtactggttcggtgctccccagaactgtgggttctcaggatcttggacatgggggaggagctccagctagtccctgcagaaattgtctttggcttgatagtggcaggcttggtggtgatcgtggctgagaatgcaaagatgccttctttcagagactagactgtgactctgtaggcgaaggccttctccatgtcttcccactgttgatcacaataaaaaaagaggcattccatggtcccaaacagtttattattgtcatggtggaaaatggatctgtagaaccataccccacttctcagggttggcatgagatgatataccttttgcagggagaaatgtatgggaaaggaagcttattggaatgccctctcgacctctaggtacctcattggcatagagaactctgttttgagcttacatgactgtagtcatgcctcttggtccgagatgttagagatgcttcatctatatgtggaagggcttgggcattgcccttcagctactgataaccacaaatctatagggggctatggctatatgacaggggattggtgctgagagcaggcgaagctcctactaaatcttcagggtctccagggcatcaagcctttggctcaaccataccttacagtccaccaccctgcagtaggagatctgagacttagggtcaagtaaaaagaggtgtcatttgctactttccttcttggccacatacccaataccacatttgttggaactgtggctgccagcctgccttgtacctcaaaaccccctttccttgggtacatttagaaatatgtttcatccatggtgtctatgtaagcacattgtgatctttcccagaccagtgaagaagagctgcaccatctcctattttccccttggatcccagagacttgacaatagcataataataatgtggtgctagaaagagatttctataaaacaaggacccgactttgaagataggctgctattttccaagctacattttatctccttggcaaaaatgataaatagttttggtttttggtattctaacctataaaatgatggcaaaaatatcaatgtcatgaatttcctgtgagtgttcaaatactcagcctatactaagttttagcaataatttccatatatttgaaatcactatcaaattttgacctggaaagagaaataaatatttaagtatcaatacatttttttctgagaacctatcttaccacaggatggtaccaagtatcaaggtgttaagtagaatcatttatcatagagagcttgtttctatgaattatgctaaacctatgaatgctttttatagtaattaaaattgttttctaagttacaattagaacatgaccccctttctctctccccaactcttcccatgtaaccctctttgctttcatccaaattcatggcatctttgttatttaattgtagatagatagatagatagatagatagatagatagatagatagatagatagcttgtatatatgaaacacatagcattcactgtgatattgtaaacctggtcagtccatatagtgttacctgtatgcatcttatgccagggctgaccactttatagcagataacaattggggagttcttcctcagggaaaaccatttctccagttcttatcatttccctattgtccatagttcttcctctagtgtgaacctccttatcttccccagttgcatgttagtatggccattggtgttgtcattgttcaggtcttatttaggcagccatgttgatgagactctatgggtgtaatctctctggcatttctaggagacccagtctcacagaaagcttgctgttcctctgtctctcacactctttcctccacttcttcagcaatgatcactaagccatgtgaaataagatggcacttggctgtaatgacacccacacacagtgtgaggactgtgagaggttcctaggaaggtcagtagatttttttccttttaaacaggaaaagaaaaagatgtcaccaataagccttcaggggaataggaattacatatttgcctacacattattggcttatcctaagtcatggcaaagttcagttttattttataaaagtgagttgtcttcataaatatcagcaatttcctttaatatcaaaaataatctgtaaacttaaagtgtgctagatctcttgcatatataggcaagtgctgtgattgacttcaaaatatagtcaaatggaaaattgctctttgaaatccaataagtaattgatgatatttttgtcacagttttagttggcttacctctgcaaaggaggaggattgttgagtgaaagggatgagggaggaacccaggcaatatatttcaatgacttcctgacataaaagccagtatgtatgagcacagaagagaatccagcatagttaccaaatcctttatgactttccatgtaccttctttgtttcttgttatagtttcattctctcttcatccccctgtaagagtagatccagacaaattaattgaaatactgtaatatgcctagatcttgcctccaaggctaaggcctagtccttgatctgtttgccagtgaggtatacaatgtaggcatgagcagcatactactttactttccagcccttattacttattacatgccatcagtcctgactatttgaaaggcaagacagaaggaccatttaggtctgggtctttgaaggtacacaagacaagatcatgactttatctagaaagaaagaaagaaagaaagaaagaaagaaagaaagaaagaaagaaagaaagaaagaaagaaaaggtttctttgttttccttcgaggctatgatttattaactaaggaaacataaaatgtgttttcagattttacttcagattgtaatgaaattttagaaccttgggacaattttcttccaggtctctccttggacatgaatgaaagtcatgagtgttgtactctcatgtccttgagtttgagaagtttgtggtcaatttttaagaactgtctgcaaagctatacatgagggtgatcagtgcttaacccaatattggtttagcctttcaccttttcttttaatttactttacatcctgatgtaacccttctacctcctctctccctaggcctgccctcacatactgctaggcctgccctcacatactgctccctcagtgccacctcaccttctcctcagagaacccccatgggtaccaatctacctggcatatcaaatcatagcaggtcgaaatgtattctctcccattgaggccagacaaggcagcccagctaggggaatggcatcaaaagcaggtaacagaggcagagacaacccccagtcaaatttttaagaggcccacatgaagaccaagctgtacatctgctacaaatgtctaaggggtccatatccagcctctgcatgctctttggttggtggtttagtctctgtgagccccatgggctcaagttagttgcctctgtagagcttcttgtggtgttcttgacccctctgactctttcgatccttccccaagtctcccagagttctgcctaatgtttggctgtgggactctgtatctgtttccactggctgctggacagttatgctagtgtcttgtttgcaagcatagcagaatatcactaagagtgtcaggatttagatctcccatgggggcgggggtcttctaaagttggggaagtcattggttaacttgtacctgaatctctgccccatatattatccctgcacatattataggtaggacaaatttggggccaaaggttttgtgaataggttggtgttcccctccctccactggaagtccctcctggtccatcacctttttaaacagacagcttctattatatcctcctatttcattcatattagtcaaatcattagggaaaatcaatcgtggtatatccagaaaccctcccctcacttccatttcacttcttgtgtgccgatgtcattgctcaatattcattttcttcatagttaaacggggaagaaagtttgaatgaacttttacaattccaaactgtctgtatagccttgaacaagacatactatctctctggatgtcacatttctgttatccaaaataataatcgtcttctggatggtctgagaaaactttccagttccacaattccttaatgattatcaaaaaatatattcaagcagtggttgtcacaatgcatgacaattaacattctaaaattgaaattaacaaaggatattttttccatgttaggagattttacgaatttcactgtggaattttaatgccttctggtcttgagaacatttgaatcattcaagatgctaatcacaagagatcctcttgcttatcttaaaactgataggtagaaagaacctaggctatcttttccacttgaatcccacatttattctttaatacccacattctaccaacagaggtaacatctcttgtaatctcccttctttggccactaagcagctaatgctttccagcaagtgatggagtaatgttgggaactaaaaagggggtccctagggaagggggggactcacaccccgccagagttttacctattctctggtctgtcaggcatgggagagctgccatacactttccactcatccctgggtgggcattcaagcctctgacccactcttcagcgggtggccaaggggcagccctacctggggatccaggagctaccttgctaaagccaccagggttatagaagagcgggacgagggaagaggttcccaacacctgtgagagtgcatgaagcggatcttgatgagcagagactctctatggttttaaagctttattatagaaaggcagggagaaagagagaagggagagagagagaaggctagagagaaagagagtaagagggagagaggagaggagaggagaggagaggagaggagaggagaggagaggagaggagaggagaggagaggagaggagaaggcaaagagaaaggaaagagaagagaagacaaagagagaggagagagggggtgagagtgaggggtaagagagtgagaagtaagaggtaagagagcaagcaggggctaaacagccctttttatggtcctcactattgctaggtaacaggggaggagtttagcttgaaggtcagaagcttgggccattgcctacgtgactactgaccatgcttctcttgtgggggttgtggggggcagtaacttaggcaggagccagagttccaggagcatgagggaacgcctaccgtgccatttaggtgaattatcacaaacggggttcagacctcagctcaactggagaccatcctgtctgtgcagagcccatgccccacagagtaatggcaaagagatggctgtgtcctcctgacagagacagagtggcaaaagcaatgacaagacattttgtgttctcattttcttctcttgttggaaccgcttacagaagaggcacagcccacagcttacctgtcaagttcctgcacccaccatctagggaagtagagacagccaagaaataacattcttctcttttctgagatgagatcacatggggtctcccagaaataaggaaatggccccttcagtctttgttgatggaaataagccagggaaaagtgaaaaataaaaacttttcttcattaactaagataccacaaaaggtatatgggatataatatgccagcccaactgcagttcattattagaaggtagacagatagaaatgggagacctttagcaaaagtccagagaggccagcaaacacaagggtgagagcagcataaagttttacggttctttttagtttacatttttatgtttaaaaagtgaacatttaaaatttaaatacatagttttaagggtctaatttttaaaaatcttgtcttttccttcatggcaggtcttagatgaagaaaaattaaagcagttggtgttgttaccactctgatgaatcacgtgtctgctactaaatgacaagctatatttaatgaatattttttattcattaaaaagcactcttgtacagtgaatggaatgttaaccttcttcagaaacagtctgcaaagaaacatttattctctcacgattaacattttcaatataatccgtgaccatattccataaaagaaaacataaatttattttacaattaacaaaaggagcctattcactgaaaacacagggaaagcattagagaaaaagatgtttgacctagacactggtccactttgccttgtgcagatggctggatgtttgttaagatgcagaagtcatgaaagaaacaggcaaaccgaaggaagaggggtgcatgcattgaaaatactctccctttctcactgactctcattgtgagatggaaacttctcaaagtaagagttcatttgcttggcttaatggtgttaattaagcctgggaaaatgcctttattagcaggtacttttgaagtgagtaaattcagagttggacacccatgatataaccaatatttgatatcttcagggcttaaactcctctctccataaatcaactggttttgagtacacggggatatcaaaagagattggtccagagactttttaggggctgtcttgtatactcaaatcagggacgttccagatcttctgagattctgccttgtacctgtgatatcattaggaacccagggagtgcactggctttggatggttccctcattcttcattagctgtgacatttcctgtaggcatcctgcctgccctacctgcttctaatgctatggtgagagaaaagagaaggttagttccatagatggcagtcatgtcatagttctttagactggttcagctgggaacacaccacccacgccctcctggtcctctatagacaagggaccaagtgcactctggcaataaggaaggagagatgttgatattattattaagaaaaaatgcacaacattgacttctggaatatgctattagtaagtagtaaagtatgtcttttggatgttcttatttttatgtgcatatatttaaatcctaagtactgttcatatggagtatatggtcttagaatcataatataaatataaaactacttcttagttcaaatgttcccttctgccttttcatttcaaactttctagtaactagttaccaagtgttggtcactccagcttcttccagtgttactcagcctgaaccagagaaggagcaacacttttttcagacagtgaagagctacaaattattgtgcattttggagatggaacaggaagttaacttaattttttaagaagacagcttctcattatgtagtgcggactgaagctcagactgtggccatgtctgtgtttgtgactttgactgtggctgtggcagtggaagtggctgtggcagtgcctgtagcagtggctgtggctgtggcagtgcctatagcagtggctgtggctatggcagtggctgtagcagtggctgtggctgtagcagtggctgtggcagtggctgtggctgtggctgtggcagtggctgtagcagtggctgtggcagtggttgtggcagtggctatagcagtggctgcagctatggcagtggctgtggtagtggctgtggctgtagcagtggctgtggctgtggcagtgcctattgcagtggctgtggctatagcagtggttgtggcagtggctgtggcagtggctgtagcagtggctgtggcagtggctgtagcagtggctgtggctgtagcagtggctgtggctgtagcagtggctgtggctgtagcagtggctatggctgtggcagtgcctattgcagtggctgtggctatagcagtggctgtggcagtgcctattgcagtggctgtggctatagcagtgtttgtggcagtggctgtggcagtggctgtggcagtggctgtgacaatggctgtggcagtggttgtggctgtgtctgagtctgtttctgaggccacagctatggctgtgtctgtgtatgtgtctgtctgtgtctgtctgtgtctgtgtctgaggctgtagctatagctatggctgtggctgaagctgaggatgtggttgtagctgtgtccatgactgtgactttctctgtgtttatgactatgtctgtgtctgaagctgtggctgagtctgtgtctgaggctgagactgtgattgtgactttgacagctatggctgcgtctttgtgtgtttgtgtctgtctgtgcctgaggctctacctgtagctgtggctgtggctgaggttgtggctaatgctgtggctgaggccctgactgtggctctctctgtgtctgtgtctgaagctgtgcctgtgactgtggttgtgtttgtggctgtggctctgactgtgaccatggctgtggtagttgctgaggctgagcctgtgactatgtctgaggctgaggttggcatggaatttctaaccccataataactttgaactcatatttgtgatcagtctacctcagcattccaagttctgaagtttttattgtacaagcagtctacaaggctgaaaatctgtgtgtgtgtgtgtgtgtgtgtgtgtgtttgtttgtttttggttttgggtttttttgttttttgttttgttttgttttggttttttggtgcatgtctactaagtgttctaccaaatgagctatatacttccagctcaattttggtcaattgcttcctttcagaaactaagaaaggggcccaggagatggacccatgaacccataaacaaggacttgtaaaatttctttataaagaaacaatgaaaattacatctccaggattctagaatggaaagaggtaaaatatttactattttccaaaaaataatgaattctgaaagggtgggccttatccttattcttattatttatttgttggttggttggtttgttttgcttcacaaactatgtggtctagaaaagtctcaataaggttcagaatgtctcattattctcaacatacaatgagaattgctagaggaatattaacagtatatgcctccctgtcctgtagtatggaatagacaaattaaagtctaagggatattggttttttttttttttttaatagcaaagcatgtatggtgactttcatcagcattaagtgtgctaagtgacaagagaaacacacccagggtaaataagtttatgtatgaccaaatgtgaaagactaatgatgctttaaattataatatatggcttagaaagttcagttcctgtagctccccagacttcagtatgggctcctcattgttttgggaagacctagtggacaccaataatatttaatacttacgtttgttctctaaaacatagtgtgacaattcaatggctagtcatatctgggagacaaatagatatattacaataaaagatggtgaaaccatggaaatgggtataaaaataatcctatttaacatcaatactaaagatagggggatataatcttcaaaacacactcatcgatactgagtttaaaattaaactcgcatctgcctccttcagagccactcactatagtatgaaaagcaccgtgaacacggtgttagagaagaaagaatccacaaaattcctcatgtaaagtcacgactacgtagtaaaagactactcacttttctccagacagtagaaataagcaaatttcattcatgggttccgtaatctgtaaaagtaacaatgagtttttttttttaatagagaaaataaagtaacatttacaaacaaaattagcacctttttttttttaacttaaaagcaacgctctccagttctgattgttttaatctttaagtcaatagttccatgataggcagagcaaaggggaatgaaaaaaaggcatctgaggaagtaataaaaccaaaataaagaggaaaaaagaaaaaggaaccagaaggaagcggttgagaaagaacacagaaatccaggctgtgagaacctatttacgatgtagccactagatggcactgtaacgttaggtgctgtctccgcttttttttctttcttttttttttaaaacaagctatttgctgatttgtattaggtaccatagagtgaggcgaggatgaagccgagaggatactgcagaggtctctggtgcatgtgtgtgtgtgtgtgcgtgtgtttgtgtacgtgtgttctgcccctatgaggctgcagcccttgtgaagcacttggtcaccttttgcaagaaggaatctgaatcgttgcaacggaaggtgcggactcgtcgtggggagatgttgctcctcactgcagatggataattttcctttttatcaggtaagccatctgattatctcattgtgatcttctttggtggtgaattcttacctggtaaatccttggggagaagagcccacggaaataagagttggggcagaacaggaagtctatttttgcatgtgaattcggaccaactgtccccctcctcctcccctcctcctcctccctcctcctcccctcctcctctcttcctcctcctcccctccccctcctcctcctctcaaccttccatgaactgaaatcaggttcgttttgcagttcagcattttgatagaaggatgagattctttggcctaaaataacttggcatcgggccagctcttgcctttgcttgcctgcatcctcctaggcaaggtcttgtcttgaagccccctctgtcattggtgctttggaactggattcgatggctgtgaatagaggtcatatcgctcctaaagatgatgcaaaaattagacctcacagaggaagcaagaaggcagcggggctgctcctggatcctagcattgcaggggaggcagcttctttgggcagaggcttgactcttggaaaggagtgcctcctccccaggaccagaagggacagatgctgtttcccccctgactcttccttaactacacttgcaaatgaactttagcaaagtgataaagagtcccgggtttcagacaatgccgggtgggaaggactgacttacttttaattagacagaggtcaccaacggcaatgatactgcagatgtgctccattgacacaaaaccactgtggaacctcttgctttctgaacagggcaaaaaaactgttttcttcgaggtggctgtgaggcttttccacgccccttccttacctctcctggcttccttctctcctacctgtttgaggtctggatataaagccctcttgccacatctacaaagtgtctgccgcccctcctttcaacaaagcagtccaaataagcttgtgagcagagacgctttttctttcgtgtgtctgacggaaatgactgagtcaagcctgggtgtctttctgttcagatgtgcatgtgactaaatattttctaagctgcagtagcaaagtcgcaaattgccaaacgctgttgctatcacacgtaggaaatgtggcttctcctcgggtatggatctgcaggtctgcactgtctatctggtgcgctagtggagtggcagagcgtgtttctccaacccgcgctgtaaatcatcccaagaggaggatgccaaacaggaataggaaaattccatgctgaggttgatgcttcctgattctgtccctcctgcccaaggagagccagggtagctcacctctcccttttgctgcaatacctgacaggccgggggtgatgcaaatgagagcttccacacacgtttgcctcctcgggaagtctgacacttccctttccttccagaaacagattaactgtggtggaaaaactcctcacaaactccccttcctggctaccgactggtaatgccatcaaacactgcatctatcttcgctggaggagtgttgcaatttaggccttcctccacaaagctccttgaagtgatttttagatggtgtctattttaggaacataatgtgtagacaggaatggatggccacagagatttacgggaaaacaatttcgcggagaagatgaggtagcattataaactgccaagacccagcacaagtggctggttatgtcccatagacctggcagtggcatctgctcttttaaactccctgccactcacttctgtggcttttttcctcgctccattacattgtttgtttttggttttcaaatgtctctttcaaaggcttgaaccggagagttcgttctggtaaaaataacacattggactttgctgtcaatactatgtctccatttacgcgattcccccttgctgagaaatttgctatcaggagactcatttattacagcaattcctctttaaggcatggcggagagggtttctccatccgtggaggctgggaaggcatgattggaggcctaattaattatacagtatccaagaaggcactggggagaagagcaaacaatctgccttcccatccaaaaagatcaggagcaagtggacagacagaaggacacaacccaaggaccagaaactcaggactccattccactgagaagccttctctcgtggagagttgctcaaaattgacacacacacaggcacatgatgatcagaacacattatctgagtgttcctgatgatgaaagttagcctgaatttaatttccattctctgtggacagcggagatctgggtagtcctcgggggccacggcagtaaaaccaggcatgccaagttctatccaagttgtcctctccctccaggttaggtgaagaagatgaaatccgccaggtactcctttccattccccttcagcaagaaggcctgatctgacttggaagagtgggagggggagaaagagtgcagggaaagaaagagaaccacagcaatccatgggctgagatgtgtcccactctggatcagtgcagagaagctcagggacgcaattacttccctttgaaattctgaaatgtcaattaagcggaatttacagctccgtttgtgtttctctggcttagggtaggctgtagaaagagacttactcttggtgcacacgtgtctgaaaaatccagatgatctacaaagtggctccacttgtgcttgtaactcttctcttttatctacgtacctagaggtcaacttattatagagaggtagtacttactttgctaagcagtacttcaggaatgtaagaactatgtaatattgataatttgacaagggaagaaatgaaggctatgagactagattgccttgccaaatgttacacagataagtggcagagcacaaacttgaactcaggcagccttgccatagcttctgtattctagaacactcatgtctgtgtggtggtagaaccactggttagccctgactccgaaaaggaaatcagcccttgccagcataactcctctctatcctttcatgccaagatgtcaaaaagtgacatccaagttaaatttttttttttacaaagccctctagttggtgtccgaaggtgttgaaagatggaaattacaaggaaattcatgcccatatgttgccttcagtctagtaataaagttagaaaaaaatgcacaaaggactatcaaaggcccttaaagaaaaagaacaccgcataaagagatgataggagagaggggcttgaagagaaaaaggaggtagaaatgtgaccttgtagttgagacatctaaatgagtagagggatggagacccttgcgtataagaaaggaacaatgattgaaaaactgggaacagaaaccgcacagaggtcctgggacctgggacctgaggtgatgtgagaaaaggaccctctgtatgggtgggtctatgcagaggtaagaaagagctgggactaacagacacccccatccccacatctgtgggttcctggtggctggactcagactcttaaagtggtttctattaaaaagcatgagggaaattggttgaataaatttaagccttgtattttatattggcaagtttctaggtttagaaaagtaatctgttcatatttacaaggagcttttttttttttaacaacttgttaagtctctgaggaaaaatgctgtcattcctagaatgcagtgatgttggctagaaaaatgttacaacgcacacacacatatacacacacactcacacacacacacacacacacacatgcacacacatacacatgcacatacagacatacccatacatgtgcacatacttgtgcactcacacaggtgcacacatgcacacacatacagactcatgcataccacatgcacatacacacacacacacatgcacacacacacactcatgcacccaacacaggtgcacacacatatgcacaatttgatgaaagattctgggtgaggttattggtgagaatgatacagactatacatacaagaagaaacatgtagaccggagcttatctgcccctgcagttggcacacaacagctggccagtggtgttcaatcaagtcatgttttgcaagtaaatgagtgtgctattggaaaaccagagctcagcttgaaattactgctttgtttactcagaatccacattataagggagtataacgtaaataagaatcattttagacttcacaactacctgttttctccttgttctcagatcagaccctagaattttctaagagatgtggatttcaacttacaatagatcctggtttcaagttttctcctatgagcaataaaaaaaactcttgttcagctcctctggtactcccatgataacaggcctctagctttaatgcagtatctacggactttctgtttgtttgtttgtttctgttttttgttgttgtttttttttaatatcacctgtccttctttagtaccatataggcctaactctatgcaaaactggacattgattctgttttgtttgcagtatgtttttgtagctggctcttttgtttatgtctctggttcatttgagtcagtttcttgtatgcaattcggcttatggctcatgctccttgctttgacttgtaagcatctacttagtccagaactctttcttgaaaagactgcattctctatccaagttgaactttcttagcaacacggccggtgattcatttgtgctattactatcatttattttacactagatatacaagacaccttttaaaggaaatgccaccaaaaaagtaccttcttggaaaaccttacatagatttaaatctgttgggtttttttaaatgacccatattctcccattgcaacctttgggttcaagtgtaccataaatttttccttctccttgggaaagcatttgtttatgatactcccagaaagcaaaagtgataattaggtacagcattttaatgaacctaatcttcctgagaggagatggctagttatttcccagaagggtccctcctgcattgcacgtcctctgctgcacacatgtaagtgatgctcatgcccttcattagacgctgcttagtcacctttgtctctcaaatgccattaggggttctgttgtacttgtaattatacttctcaaatagagttggttttattttagatgctttgggcctatgtaacactacactgaatgctgggtggtttaaactataggaatatagtttgcatgagatcaggctttgagatattgacaggtatggttccttgtgatgcttctctgcttggcttgctggtttcatgctctgtgctctgacatgtttgtttcttctgtgatcctgaattcacagtgcttgctttgatgcctaaatttccacttagatggagaaaagttagattgtgtctaaccgtgtgatccaactattttaaaggccttgcctccaaatacacacagatatggagtagggaagtttggctttaggcaaatgaacctagaggtgaacacgattcatcttgtgatggttgcctttggcttgcttatgatgactaaaatacacaaggaagactgaatatcaattcagtctttttttgttgctgatgacatgattaaaattgtattaaggatggattttcgtcatccatacccttctcccacaataaggagtagtgtgggcatatgttgatatgctaatgttgtaattagctaattaccattatatttaaagaagcagcataaataacatgatgtggagaagtgaatgcatatttagagccaccatctgtgctaagcattaaggcttttactcattttaattacaagaggaaccaagatggatgcaaaacttaggagcttatggacaattttataaataaaacgatcaattccaaagtgtttctataagcaatagaaactgggtcaggctcatttaaacaaagtgcaatgtcttgggaaaatgttgcagcctagagcccaacatttgcaccagaaaatggcaggaatgaagcaagcagtaaaagtagttggcccaaggttggacttccgatgctgtaatctcctaaaacaaaccagctaactgttctctcttgctgtttgtggtacagatttggaattacagttctaacaaatcagcgtctggctgataagtttggactatccatccaatcattttaattaataataatctggaccttcaactgtcacttctaccaaaagattcctatgtaggaaaaaacattgcaacccgatgaaacttaaaacagtttacatatactatgttaaatcataatctatgtaaacaaaaagatattacagggccattgaatgtaacatatcaaataacatttaattctgtacaaagttgctgaggatacaatagcacaaatcccaaaaagatacaaatgtttgtttcccctaaaaaaaaaatgaataaaactagtgttagaattggtagagtctgctatatgcaattataatttctattttcttgttataatttgtttaaagtcagaattatttttctccttttaataaagtagagaatgttcattatgtagatctaaaataaaaaccataaggaaataacataactttaaaacattgcacaataagcattagtactaacaatttagttgtgcagtttttcccattttattgatttgtttctaaattctttagtttgtaatggcagaaaatatttttgaaataagaacacactgttttaaaagttaaaataggttcaatggtagagcacttgtccagtatgtgtgagaccccaaacttaatccttaacattaaaaaattaaatatagatgttagaacacattatttatcaaatggaacttggcaaatcaatttataaggataagcattacataatcaaaatagtgaccaggagataaatgagaaaatagccaaatattttaatattttatcacctagaatattaattaaattagtccatatttaatatattaatttgctgttttagcaaaattgtaaaaatggaaattttggaaagaaatgtgatacaagattagatttgatataagaaacttgaaattgtgtaattttgtaacctaatgtctccagtcagacttcagaaaattttcaggaaatttacttgtacttatggaatgtctattattggctatgggttttagtaggggaaaaaatgagtatacactaaaaaaactaaagatagtaaacgataaatgaacaaagaacaccactaaacactaagccagaacatgacatttttaaactgtgtagaactcaactgatcacattggcaaaatcaatctatgtcaatgttccggtgagactatagcaaaggcagatgcatgaggctgagccagagccattggcagagcgagggctacatcctctttcaagaactgatccttgaaaatatgaaatcgcctggtaggagcagggaaaggcaccctgtaaacaaggatcgatcgttaatatcatggattaatatccatacttagattattttaaggaaaattttaagatttaaatactacagagagtcaaataaaataaggaacccagaggtcaagattttcatttgcccactcaaagaggagaaaaaatatataaagaattctttcacataaacatgtctgtctaagaattcagtgaattgaaaatcctggaaagtgcaactttgagagacttgacaaatatcaccagccagcagttttgatcctcccctggaatgtgatatgccatttttaaaaggccagtggtattttgagctatgtaagtcagtgtgtttggtacaaaaccaacgagccaacaggttggtttgaggactagaataccataatctcagagccagctgacctaagcaggtcagatttcagatcattggaagtgcctggaagattgaacaccagtactttagggcattagagacaaatagttcaacctgtgtgaaggaaggcacgagtgtcgtttgaagaatttctagctggtaagagctagctctgaagaaatcattgagggaaggaaatgaaaataagaaggtgaacttcttcaccccactatagagtaatgaagtatcgaactccaggcaacactgtagagtaacaaagtgacaaactccagcagcttctcctccagagagaaaccttacctgctcaccaattagccacctctcctttgtcactatgactttgttgactctctactctcgcatactcttcacttttaatcttgtcgccatattgtgacatttatttaaaacaaaaacaaaaacaaaaaacaaaaaacaaaaaacattcagtcatgttatcaactcagtgtttagtctttgatgctctcaaaaatgtgtccaccccatcaagactagaacagtcttaaaaataacttttgcagttattttttatcttaccccataactttggtgcatttccttgaaaacgtttaggcatccatcatatcccaactgtctttatagtcatataaaagcactcttccttcctttcactgttgatggttatttatatttacaaaaacaatttatatttctcaatgttaatccttagtaaggcagtggtatgaagaattgttaaatttttcacagccatttgctcctaggaaaaataataatctataaaatgaaaatccaattcccttaaaggagtctgtttgatgatgctgcaaaactatttcatctggagccccagggctcatcttgtttgttcgtttttttgtttgtttggtgccccaatatcatccccaaaacacacctgcacagctgtgtgagttaccagctggactgattccatttccattgaacactatcacgccttggaaaatgtagcaggtagacaggtgcagaccattccagcttacattttaagtcattttcaaaaatgagagaagtgatcgagccatgtcaaggagtaaaaagtaattgtaaatatgtattgcccgtgacaaaaatgaccattagaatactggaacacttttctgccaccaggaattgagcagcattttaaatatgaaggatttccagatgtgactagcagtgatgttgacagactaatgaggagaggctgtaaaaatttaaactttatatagcacagtcagctaacaatctataaatgggccatgcttatgacatatatctttgtggatagtaaatcacctaactgtgcacaataagccaatgaattgcataggtcagagtacagtgggtttgcggatatgactctgacctatttatctaatgtggcctaccaggaaggagaaatatttatacttacatgaaatcgctatttcaggtttccacacacctttctgcaagaccaactgtttttcatattctccatctaaatataaattaaaatctgttaaatgtaaaagaatctagatattaggttagatatttgaaaatttataaaattataaaaacccatgccacgtaagtccttttctctagaaacatggtcattttccattaaaaagtaacttagttagcatttgtttatgattgatgttcacacaattttgatatttaatgctttaagatttctctaatttctaatagcaatgcatttaattcattgatgctaaagctttaatggttcctcaaccatagtaaaaatgtacatgctaggggctaagccattgcttagcacatactgtgtacccctgggttatatccctggcaccaaaactaacaataatgatattaactacataaacaaatttgaagatcaaaatttttattctaaggctgagtttgataactgatctgcttttaaagtacaattaattgtgtgtgttttgctattaacaggcactaatcgagattaagatgcaggcaattgagtccctttaatttatgctacacggataaaatatttatcgcattatattagttttaaagtttcctgctacaataaaaggatctgtgcatattatcactttgctaagctttagtaaacgtgttgtgaagtaagtcagcaaattgtgaatgttcatttaaacttttatcattccttactggaatgaatgtaagcattttctattgatctaactcatctcctttctcctgtttctcagtgagcagctgacctctatgttcccatcagcgctaacatgcacattttacttaggctgtcgagcctagtgtgatgggacatgcctggaatactccaactctgtaaatattgtcattttgtttattcttatagggaatagaagggccgttctttgagtggtgggatgcatcagtaactttaggtgtgtgctggagttttcagtcagtacagagagagtcagtttcctttgctagtggtggccttgagccagagtggagagctgaagatatctcctggctcataagagcctcagtgaggaggggtcgtcacccaagctccatcaccaaggggaaggcacagtggactgaggagaaatctggagcttgtcgatagaattttcagatggttagaaacggatagcaaccaaaagtgaaaaagacaactgccatccaaaccaagctgaatgaaatgggatgtgcctgaagaccatccaaggaagtccaccttggccactgaaggctggggtatgtggggagatgcattctccccacctgcgcaagacagagcagggatgctagacattcaacagtaatcatccgatgattcggggaaggtttaggatgggtagtaagagaaacctcaccaataagctgctgtgagatgaaatgaatggccattaggagaactggatcgggggtcctagaaacgagtcctgatccaagtcatagcatcactgaaagggaggtcggcactcttaaaccaaattctcagtacaccgaattattaactaggaggagcaggtgggggcaatgtggggctgcgggggaagggagatggtcaagctgctgctaaggcaacatagcagcaagtaagaaagcaaaagaaatcagagagcaaggggcaaaagaagcaaggggatgcttcttcatgattgagcttttaaaggggaaagggggaaatatgtgctggagtgaattagtaaatcctgactggacatattagccaaataattaattcttatagttggccctcggtgtttcatctcaggaatgaatcagtagccaaatgagggaatagaccttggggaaagggagaagaaaagatgtgaaagtgcaaacctgtgtgtactgtttgccatgccagagcctgctagaacaccttcagcaagaagatcactatgaattttgaggccatcctgggctaaaaagtgatatgctatctcaaagcaaacaccaccaatggcaacaacagcaacagcaacaaaaatcttccattatttaaatgtggcgtcagggtttaaaaatccaatgcaagatgactggcttccatgagctgtctaggtttacctcttaccctgtggcctatctatcctgtagaagtaaatgtgtctgtgagttattgaagctgcttccaagccttcgaaacttgttcattctctgtgacatcatgacctctccttcccctcagtaggagtcactttccttctgagatgtgcctaggcccagtgtggacttccatcacagcaccaactgtatgcactgtgtggtgtctgcttgtctttccctccataccttctttgcagtgtgatgtagttagtatcttgtgtgtgacaaggtactgtcaccactgtctgtttcaactttgtatgttcagtaattgcccaacagaagtaagttaaatgaaaccaaaataagaagttttacattattcttacatatttactaaatctataatttaaggatgttttataaataccattatataaaatatacctgaaaagcatataataattttatattatttcacacagattctgattatatcactcagatacactcccatcagtatatctttcttctaatttattctgacatttttctcaagtgacaatctgcagctcttatgacttaattcactatctcaaaattttaatttcttcttttacatttttcagacatccattgatgttggcttggagccaactcttaataacccaattattttcatagttataaatttactacttataatgctattataaaaacagagcatttgtaaatgcataaagaagaaaataatattcatgatctcataatttcttattctccatattaatcatgggtattgcttaatatttagtattttactctacattattccttttgtgtaataggattttatgatgttttcaatatatgccatgtgccatatattcaaatatttttagtgccatcagaaaaaataatataatgttaaaacatttttactaatagttttaaatttataaaatcactcctatcgtcatggaaaatttgtcagatcatgacttttgagagaaagtttaaaatgtcaattactttaaaaatatcaaacatgggctggatagaaggcttggtgattaaaagcagtgactgctcttccagagatcaggagttcaattccctcaattcccagcaaccacatggtggctcacaactgtctataatgagatctgatgtgccttctggcctgcaggtatacattcaggcagaacactacattcataaatattttttaaagaaatatgaaatacaactccaaatctgtgcttttgctgagacaacggaactccaacaacatctttttagcatctacaagataaaacgagaagggcttgggcgtagactggggttaagaccattctccacccctctcttgtgcttggttctccttggctgaaaagcacacataggacttgcctgacactttctttcactctgccaccatttatccaaaacatatgttatagttcattaaactacttcaccttttcaactttcaagaagagagacaaaatgatttttacatcatcagtaataacctgtagaattacagtgtctaattgatttctcatgtttttaactttttttctagcagcaactaacactggactcttcagctgaacatttttaagggatttaaaatatccattagaatttctctttcataacatattgcataatattagattcagaagagggatagtaaagcgtttcccaaattgttatttattcttcaaatatgtattgttgaaagtttgctctcccctaaggcaaaagtgccattaatatttttcttgcgatctaagaaccacattgtatattaaagcagactaacgtagtattttctcttttattataaacagattctttctttccgcatcccagttcctctccacttcaatctggatccattccgtttctgcctgtcattagaaaagaacaggcttctaaggcataacaaaataaaacatacaatatgatgaaataaaaatcatcacgtcaaaactggacaaggcaaacaaacagaagataaaaagccacaagagtaggtttaagaatcagaggtccactcattcacacgcaggagtcccatagaaatactaaactgaaagctgtgatatatatgtgtatatatagtatatattacacacacatatatatcatatatacacatatatataatacacacacacacatatatatacacacagaggacctggtatacacccatgcacaccctgtgcttgctgcttcagtctctatgagtttgtatgagctttgctgagttgattcacagagctgtgttctcctgctgatctccattcctgactcttacaccctttctacctcctcttccaaagggttctctgagctccaaggacacatcccatttagagctggcaactctgtaatattaaaaattactataattattagtttaaatgaaacggtagtcctttatatccaccgtcaatgatatatattttattaaatataattataatatgtaactctaagttagagataatcttaaatttcatggcttttaaaaaataatgctatgttcaagttcatctgtatgaccttgattagtggtgagttcagggacatttaagtgaccttgcctttctttcctacattagtaatagaatagatagcatctgtctaaagtaggactgtaattctgtatggctctcccctctgggttatatatccagaagacctaggaaattggttaatttcataaaaattccaggggagggcttgtttgcgagttcattgctcttgccagggattgaataagtgttgggctcctgacctcattaatgaaatatgagggcaatctgcagtcaggcttctggaagaacacttctcacctgggcaaacgccacaaggggaaataaaggtcttccttattactgctgcagcctctctagatgtcacactcttgagccgcagccatcctggtcagctaacattggtgctgatggaaccacagagtgtaaatccttggaaaaagtattgaaccaccccgtcagctcactggaatcctcatcttctgcactttcttagcttgtgcgatttgaagttgaggactcctagtcaaaagaacagttgactgtcttatatgtcatattatagaggcctcttctaaaattatatttagcattatatctatgattcacatttaaagtagtcattttaacaaatcagttaagacactgaaccaataacactctttaggccactgtaatagtattcataatattggggctccaagtgaaacaggaagtttggcatagtaataaataatttcctcatcattcattctcttatttgctgttaactaatgtaactgagtagaaaaataattaagacattacaattctaaaacaagaaagaataccagaaggatggctgagcaaccactgctatgcaatcctaacaccttgagtgaagtcaatggaacctatactgcaaggatctgagaggccatctctgaaagttgtcctctgccttttacaaacatacatcatggcatgagtgcaccatagttcacacatatgtgtaacacacacacacacacatatacacacacaccagtaataatataattaaattcataaaaagagaaatgaatgaaattttgaagatatgtaagaatcattttattatctttttgtagttcagtgcttttaaatacaattagagttgtaaaaacatcattgcaatcattttaaaacatgtacatcaagtatcaaaaataccaaatatcgacttccattcaatccaaggcaatctggatttttaccttctgcccctgaacatttgctgtctctagacatttcataacagaactattcattatgtcctattgtataatccattattctacctaaaatgtttttgagatttatccaacttgtgtcacatacattattttccactcattctctagtctaagtgtcatcttgtttaaccgtcaccatctattgatgatacatttgtgaattgtacattttggatactataaataccattgctaagggcatttccatgtgagttgggcataagataaaaactgtttctcttaggacttaacttgaggtcttttaaaacatcacacataagctgagaaattatctaattttctatcagaattactctcccacagataaagtctttggagttgtttttattccttgggaaatactttttcagggtgctggtgttattgtttacctgcacacaagcagcactgtgcttccttatctacagaggcaagactaagtggccaatttatttcaaaccttaatgagaaagaaagaaggaaggaaggagggagggaagggaagggaagggaagggaagggaagggaagggaagggaagggaagggaagggaagggaagggaagggaaggaggaagggggaagggggaagggaagggaagggggaagggaagggggaagggaagggggaagggaagggggaagggaagggggaagggaaggggaagggaagggggaagggaaggggggaaggaaggaaggaaggaaggaaggaaggaaggaaggaaggaaggaaggaaggaaggaaggaaaagagggaaggaggaagagaagacagacagacaaagacagaaagaaacaaagaaaaaaacaaaagaacaaagagataaagaaagaaacctctgttgaactgttgaccaagaatcataaatccattgataatatgtaccctactctgcttttaagtgggaataccttgaagggaaatgatcagatatagaaaagatatagggctgaattgaaccttaaagctgattggatggacgcagaccatagtcttgtgactgttttctgccttatcaaatttaaaccctgatttacagatgaagacacgttgtaggaaggtagaacaccatgtctttgagttttgaagcagctccttcttcctcatgggctccctgtaccccagtcttcatgtccactctctattccaggattttgattagttttacagcattttttttctccagtatataagacatctaccccttcctgataaagcctcccactgggctcaataacgtctcatccttctacacatagacaagcttcttcagaagagatttctcaaagtctctatttcttcatgtgaagttagcttttatgccagcctaattgtattgcatttcagcatgacacatccaatacagtgattactggcaaacaccatggtgcttcagggccctccgttcttcccaaactagattatgctatgacgttatatggtgttcgctatctctttgtaaaattctctcttccttgtctttcttgtctacaaaatggagatcactattttaattttcttcaaagattagctattaaattttaagagtcaaaatattcccaatgtctgccatctaaatcaagaatatattgtaacttcaggtcaacctgggatattcaagaactatgctgtaatgagaccctagcagatggggagtggggggacaagaaaatcaaataaagcaagaacaagaacattcattttcatcaatatatagtgtcatcattttctttagagagttttgacaaatcgtgtctctggaattctttcttctgctctccataaataccacgaatgaaattaactaaccaaagtgagttctggttcgattgactcacttattcttcattcagcagatatttataaacatggtcccaggcaccctgccacgagcagagataaagttccaactaataggattccaaggaacgatttgaggagcccagactggggcaaatcagcatttgcagttcagccctggctgcacatgagatcaagttgataatgttattttgtcttctgatttttgcttcctcatctataaagatcttgattccaatctacatttgcaaatcacagcttaaggcctcaagtcatgtttgccaagtacataaagtgatgcttggcactgatcaaacactttcaaacactgtcatcattttgttttgcaattttctcctaaatatcaaaacattatattaattttacatgcctcctatacccacagccctttcctttgcttcctagtatactgtcttgcccttgagttcaagcctaccgaggaagcttcttagcaacattttcctctcccagtgtcctggaggaattaatcattaatcacatcctcatctgtatttctgtgtttcaaaagcctgtgtgtgtgtgtgtgtgtgtgtgtgtataatttactcagtttatacagtttatattaaaatagaaataacaactagctcacaccagagagcctcagaacctgttattgcttggttcttaaggctactttagcaatcacattacttccacagtggctgtctcacactgggaaggctggcaatgcagtagttgctccatccacaaggctgggtgcctcagcagtctccatatagtggccaaaacatggaaggtatctggaaggtacctagaccttagtcgatggtggaagctaggtatttgggtaccactggtcctgatcatcaataaaggagtcagctgcagaaaccagatggtcagtaaactcaacagcaagaggaaaggtcagcaagctgtgaataagcttccttccactaagtctctctttatctagatggctaccagaatctgctacccacacccggggtgggtttccccacatcaattaaggtgaccaggaccattcttcagttggtgcttcccactcagaggatgtgaatttgtggcaagttgacattaaaatcaaccatcgcaaataatgtccagtgtgtctctcctaatagaccgtgtctttcttcaaagcagaagttgtgtgtttcacatacatacacacacacacacatacgctcactcacacactcacatagactcttacgtatcccaacccaacatggtaagcagttcttcaaactgcttcatttttttaatattattaaaccaggccatgtatacaagccctgcatggcctacaaaatagatagctctaaaaaaaaaaatcactgaaatttcattcgttgagtattactttctcagtgcctaatttctatgggcacaatatatatttaaaatggtacgttatcaaataattttgctcggacttaacaaacaaaacatgtctctacaattcaatggcttctggatactaaaaagtatcttttactatgaatgataaaatatttggaattacataaattgttatatttaatcttaataatttatctttaaatcaacaaatatattgcagagtgtaatgcaactcttcagtcttaagtgaaatccttcattataaatatgcattgactcagtaacatcatttttccgtatgttctacatttaaagtgtcaatattttcatgttattggtttaatcacctcaatactttcttaggtagatgggtgtgtcaatttaatggttatccatgctgagtgacatttaggcatacagtcattccaatttttgacattattacatcctgctttttattttatgagtaaaaaaatgatagcactgctaaaatgtcaacttcttaaaatgtataatggaaaagtagtttgctagtaaatggtttcaagagtaaaatatataaataagcttatagactaataatgatagtgattaatgcagttttattgaatacgaagttttacaagtactaaatcactaactgaaaagatgttattctctaccttgatggtaactctaccctacccctaataaacatatatgtacatgtgaatacactatcattcacatcataaattataataaaaaacagttgagagaaaagcaatgtaaataatgtgaattgaacacaaatatttctacagagcacttcattagcaataagtattttctaatctactggtactctaaaagactacttaaaatcaagaactgaatgtaagggctttgtatatcttaaatagtttttacagtctgtatttccaattgaggataatatgataatcttacatatgaaaggttttaaacgattcctgtttagaccagctatctggagatacctgaaaaggtttacaaacactagtatttagagggagcaagttaacagggattaatggatgtgatacaaaagatttctaaaagctggcattttcttctaggccaaaagtagacagaagaattttaaaatacaagcaaaatgtaattgtcattcatttgccgatttttaaaaatatataaaatgaatgtaagatgttgagatttatatctaaatggttaatggaatttttaaaagttaatatctaactctaggaaaaggaaaatttggaatattctccttgagaaaagaggttaaatagccttacgactttcatcttcatccaatatcttaaaatttcttctaaagaaaaaaaaaaacaaaaacaaaacaaaatcaacacaaacctcatgtatttacattgacagtcatccagaacagcacagagtaaaagccaggataacttgtgagtggaggtggtgtcggatcaatttcatttccttgtgagataaagtaaataaacatgtggaccaagacagaagtaataaatagtgcttagccacactttaggaaggctgttgtctatcaatatatcatctacttatctattgatcagctatcatatatctacctatgcttttaagaggcatggtctctcacccattcgcaactttttcatcagaaaccaggggagaatgctccatgagacaaggggaatcatcagtggcgactgatggtagccatgcacactggctgtccacaggctttggtgaggaaaggacctctttgaaaaactttaattttattggataccactctatgtatgtttctttagggagttatggttgcctataaagcatgtattagcaatgaaccaaagctggagggtacaataaagattcttagagccctcgtgacaaacctgagtttgattttctataactattggtctctgtgttaggactgtcccaggcgtatctgtgaggttctacttggttagtaacaactctatgagatccaggggcgatgtactaacatcttcaggaattgaaccagactgtagctgtttaataagtaccattagtcagaatgttgaaatgaaaagcaaatgcgtaagtggtatgtcgtaaaaaggtatgaatactttcagggaaaagtaggaacatgaaacagatcagccttcaaatttataaactggtccatcgaggtagggaagattggaatgtcatccgtagactgatacactttaattcaagaactactacaaataataacacagttgagacgccagtcacaaacttgaaggaaccatcaatcgtgtttgcttaataaatcatcaacaaaacattatttccatttaatgacaggaataggagagctataaactctcttcttatgaaaagaacatattttgttgcatttccccatcttaaaatgttaaagttatgtgtatgaagtcaaacgcaaatcctggtatggtgcctcaaatgtaaactctgactacttagggttcgaggtaagaggaccaagggaatcactgttcagttcagtgccagactggacacatagtcagatctaaacaagtctgtcacatagcaagactccccagacagataaaagtaaacaacaacaaaaaagccagacatggattaaaaactttaataacattgttataccactgaaagcaacaacattttttactaagagttgtactttgtatacaaaatcagcattatctcattgttcatcttccttctactattctgatgtctctgctaagtaaacaaaaaaggtaagaatcatacagctcttatgataaatcagtaaagataaaatactgaaaaggagattccattttggagatgaggaaaccaaagggaggtatggtagcttcattcacgctgatgcatgtgccccttgtgatgtgagtatgctccagttttcattccggtggacagacagacggctggagatgaacggaaattgtaggcaggtagaatctcatttgcttattcctgttttgctgactaaacaacttgatccaacttgtaaaatcaaatattgtgtcaaagagtgtaataaaacgcatgcaagctacaggaagtggtgaaatgtcaaatgctcaagcattattctttgctggcccggattcgcctttgtgcttccttacagctgatggggaaagtgaagaggcatggcatgccatagattgatccctcagcctatgcagtacagttcataactgaaggagagcacactgcaaatctctgtttaccccatgccttgtaatatatatgtgcctatagatttggttctttaacaattggttgactcattgattgttatttaatgattactttttggtttagctctacagttttcctagtaactaataaatcaccgggtggtacaatttctaataatatgcctgctgaatatacaattttgatattttttatcatacgtactttgttttctacatgtggacctacttccaagcatttaaaacaatgcataattttatactaagatgttttgtatttagaaatgagacattcagaatgttatttgttattctgtcaaaagattatataataatgtaaaaaaactgaccttctatgggaaataaagactcattttaaaatgactagcataacataatgttaagcaggtcaagacttgctctgtcagaccgaggtctttgcagtctaaattggttaaacaatattatgaactgttcaaattattgaatgattacaaggtgtgtacttcattagaaaagattataaaactgtaaaattcagtcactctccaagacccattctattgtattcttacacttaaactgtagcaacaatggctgctttatttttggtcaaacttttctgtcataatttgtaccatgaaactttaatatctcagcaatgttgttgatccaaattctagagattatgttaaaggctttttttaagaaaagattttttacctgtggcactttaaaaaatattcttaatatgaataattgggatgatacaatttttgaaagatacctacaaggtcaccaaggttttaaaacatctccctactgaatgtctaggttttgtttttttactttaactttatttataatgtggctctaaggatcaggcccacagctctgggcttcctaaacacacaatctatatttagcaatatccccagttttaaaagatgagtgattttccactttatgttattttattaatatttttactcagtatgcaaattgtttccacatgtcatccatatgtatgtgccattatttagtttcagtacttaaaacagtaatttaaatcagtagaagagcactttccagacttaaaacaagtattgaaaaattattataccatagggcaaatcactcaagtatttataatatttcattttgttgaacttttttattattctctatagtaaaaacatagaaatccttctaccatgttccatgagccactcagaaagtggtagagatgattcatttaggggtgagcactcaaagtcatttactctaaaagaaatggttatttatgagtgaccatattaacctttgcccattacaagatgaagattctctagccgaggttgagagcagcactatctatgggcataaacaaacgtatttgggaagtaatttgacagcatggcatttagcaaagcaacagttctaactccctcattagtacctatgaattccctggccataggcttaggaccagtcttaacagtgataggcatgaatccatcttgtgaagcaggcttcagatacaatcaaagaccagttggttaccctcataataatcagccctgtgattattgcaccagtaggtacattgtcctggtaggttggtcacaagtgatttaggccattgatcactttcttcactagcagcttacaaaagcactttctgggactataaatgctaagcaaaagggaggaagcttctaggtcagtcccgcctcgatttctctattttctgtaaccgaagactgcaatctcttcagcaacagggtcctatcatccacttctaatgagcaagcaaaggcgatggctatagctcctactgttttatgggcctgccactggggcctctatgagcaacgactcctaggaaagaaataacataaaaggagttctgaaaaccttgtttcctggatatgactctgctatagccaagtcacaatggctatggttagttacacaagatggagcccatgatatttaatgacatatggtaggaggtccaccctcacccaagggggccatcagcatgcacttaagtgccactgtggtaaagcaggtatttcattcaatgctgaagccacttgtcagttgtccatgctccagtaaccatcctccacaaaatgccgtgttcatggaaacagctttaattacactcagtctgtgaccttccaacaaagagacatgaaactaggaaagaatgtattaggaataagaagttcattgggtgtgggcgaacaggatgataaaaaatgatcaacacacataatggacacgagtgagattgccagaaaataaattaaaaaatacaaagaaaaaaaaaaaaacccctgaagaatctacccagctttgtgactcatgcctgtaatcccagcaccaggaaaatgaggcagggtgataaaagaccaggttagactataaagtggattacaggccgttaggttacagagcaagactgtgtctcacaaataaaaagaaacaaagatagaattatgagtaattaaagtgtgacatgaaaatgattttcacattatgtttgggccacagaagacttatattaagagcttatttcaacttaatataagtaattggtgcattaatagtgggcatacaatcagagagccaagagccgaaatacgactaaggcagaggcagaagtgcccctgagaatttggccacataagcagatgaacctattctttatcctggtgactcgacaattgctttgctttatctatctatctatctatctatctatctatctatctatctatctatctatctatctatctatctatctacctatctacctatctatctatttatttaggcctctcttttgttgttatggtcctttgcttgctctaagcttcacttcccagaattcaactctagatgactttgagttcatgtttccttgacttttctgactcattctcagcccaatttagtgttgctcagaaagagttcctgattttctcagctaactaagctaagagtcacgtggcctatgtagagttttctgtgtgcaagcgaccattgaggacagtattttaaaggagtagagggctatatagggctctagtatttatagacttcagaagtgatggaattgaagccaagaaaagtcaagaaatcgcattcagaaatagggttcagaaccctaaatattctaagtggaaagataccatcaaggaatgtccatgactgagaagctaagactccaagacagaaatacttccttctcttgcttagatcagtttaatcttggctccatctttagcaattgtacttggccatgaatgaactcactccctttcttccaaaattttggtagtataaagcagtggttctcagcctgccaaatgctgcaaccccttagcacagttcctcatgtggcgatgactcccaactctaaaattctttctattgctacttaataattgtaattttgctactattatgaatcatagtgtaagtatctatgtttttcatagtcttaggtgacctctgtaaaactgtcattctaatcccaaaaggatttccacctacagggtgagaaccactgatgtaaaggctgcttactagtgtagtattttttcaacacatacttattctctatgtaggaaccctgaaaactgactacagatatagatagctttgagatggagcctgtagatgtagctttcctttcttagctctaggtttatttatctacagaagaaattcttctaaaaggtcttcattcaattaaaatttagtaggacattctcagattccttaagcatattagctaagcgaagttctgtgaattgctcagtggtaaggtttcctttgatttggcaatgacacgtatgttaagagttgggccaggaagataatggcattgccctttcgtttagtccgtgatgagtaggtgaccaaagggaaataatggattataaatccatttagttattaatattcaattgaaaagttacagagtgcagtatgggaagcattttggtccaagatgtcagacagaagcattctctttatgattttagcgaatcggtagagaaaataaataaataaataaataaataaataaataaataaataagacacaggaggtaactcgctggacattgctgtatgtgttcactattcagtctttgccccgtagctgatttcctctctgccttccttttggcagaacagtataaggggggagcgacagcggaagggtttagattgtctgcagccagccacatgcagaaaagctgctgagtaatgcgagcagaggagggaggcgtggccctttctctgcagcccagttcacagcctcagccaggactctcagcctggagatttttggatcaccccaccttttataaacctcaaaatgatcttcgaacagaaggaagcatgaaataaatgcaccaggattctctttactcctctcaccaatatctcagtccttctttggagaccttcaagtttgaaattgtcataagcatctttattttctaaaacttgttcaatgatttctcgagttaaacaccgttcttcgcagccagcgctcaatggactcacaaaaccgtgaggctagttctgtgagttttcaaaacggcggtctcttttctgtgtcagatgaaagcaagattaccaccacggaaaatggttatagtgccgccgtccgtgcccgtgtcccggtactgcactcgcatgcagcttctgtaaagctgttgtataactttgcccttcagtaaggctgctccttttttccccataaattatggaagattaattcaatgttatattgatgacatttgttatcaatataacactgtataacaagatgtacatagcacatcatgatatgtgtctcatgtagatcctggctaaataaaatgctggtagttgagataaaatgtaattgtggtttgaaatcactttggtaagttggatggcttttaaatgtaaattttaggaaaataaaattttatgctgtggttagctatttaagaataacaaatagtagctgggcgtggtgatgcacgcctttaatcccagcacttaggaagcagaggcaggcgaatttctgagttcgaggccagcctggtctacagagtgagttccaggacagccagggctacacagagaaatcctgtaagagtaacaaatagcaccatggacaagctaacataacatgtagtagtcagctcttgggagctggttaatgtcttaggagtttgctttaaatagactttaactaaaatttacttattaaaaagttggagcagatccatagtgtgtattccctctctgggttacatacaagggtttgtaatgataataacatataatacattagttacattttataataaaattcctccattctgcttaatgaatgctacaagctgtggtaacctgaggaagacctcaaggtctggtgactgtttaaggagaactaaacactaaattcatacttgccacttcacatttggtaggaatattttttgtcatgtataaaattgaatggagaaatatccttcacttcttacaagcgcatatgctatgattcacagaaacatagaagggccagcctttctgctgtggcggctgtccagggctgcctcctgatccttagtggtatgacttaacgttttctttcttttctagagtttctttgtatacaaaatcaattgtcaaagtcaaaagtcaatttcgttgatagtaaaattaacaaggtggaagccctttgtggggattggaatgagaatggtcccagaggttcgtaagcttggtttccagttggtagactactgggaaggattaggaggtgtggccttgttgtaggatttgtgatctggggctgagatttgaggtttcaaaactctcattctctgtctgtctgtctgtctgtctgtctctctctttctctctctctctctctctctctctctctctatatatatatatatatatatatatatatatatatatatatatatatatatctgtctctctctgtctctctgtctctctctgtctctctctctccctcccttgctcccttcccctctttcttttctccctctgactccagcttgtaaatcaggatgtagctctcagctaccacttcagtgccttgcctgtctgcctgctgctaggctccctgccatgatgataagggactctgaaactgtaagcaagcccccaattaaaagcttccttttataagagttgccttgatcatggtgtctcttcacagcaatagaacagtgactaagacaaccactgaagtggaaatattcactgctaaagtctttgaatcccagattgcttaatgataagaaactgactagcaaggacagaaagctttaccaaccaggctcagagttggctttggagaccctatgtgaggtttctgctaaaattagaagccaggtttcagagtacagctttttaaaatattctctaatgcttctttaaattgtgttagaaactgaaatggccttccctttttcatttataattttgctaatctgaatcttctctttctttatctttgtcaattttaattttaaaaaggattatcccagtgttttattaattttatgtattgcttttaagcttctctctaattttcacactgattattattgtttcttgttagggcatatgatttgttcttacttttgtaagtgcttgatgtacaatattaggaaatgtgttcgattttttttattacttctttgagaactgcaaaataatgtggtttgaccatatttgactcctctcttggctctttgtaagtcaacaccatttcttttttttttacattatttttattacgtattttcctcaattacatttccaatgctatcccaaaaatcccccataccctccccctcactcccctacccacccattcccactttttggccctggcgttcccctgtactggggcatataaagtttgaatgtccaatgggcctctctttccagtgatggcctactaagccatcttttaattcatatgcagctagagtcaagagctccggggtactggttagttcataatgttgttgcacctacagggttgcagatctctttagctccttggatactttctctagttcctccactgggggccctatgatccatccaatagctgactgtgagcatccacttatgtgtttgctaggccccggcctagtctcacaagagacagctatatcaggatcctttcagcaaactcttgctagtgtatgcaatggtgtcagcgtttggaggctaattatgggatggatccctggcatctctatgatgcagtctctagatggtccatccttttgtctcagctccaaactttgtctctgtaactccttccatgggtgattgtttccaattctaagaaggggcaaagtgtccacactttggtcttcgttcttcttcagtttcatgtgttttacaaattgtctcttacatctcgggtatactaagtttctgggctaatatccacttatcagtgagtacatatcatttgagttcttttgtgattgtgttacctcactcaggatgatgccctccaggtccaaccatttgcctaggaattttatgaattcattctttttaatagctgagtagtactccattgtgtaaatgtaccacattttctgtatccattcctctgttgaggggcatctgggttctttccagcttctggctattataaataaggctgctatgaacatagtggagcatgtgtccttcttaccagttgggacatcttctggatatatgcccaggagaggtattgtgggatcctccggtagtactatgtccaattttctgaggaatcgccagactgatttccagagtggttgtacaagcttgcaatcccaccaacaatgaaggagtgttcctctttctccacatcctcgccagcatctgctgtcacctgaatttttgatcttagccattctgactggtgtgaagtggaatctcagggttgttttgatttgcatttctctgatgattaaggatgctgaacattttttcaggtgtttctcagccatttggtattcctcaggtgagaattctttgtttagctctgagccccatttttaatggggttatttgattttctggagtccaccttcttgagttctttatatatattggatattagtcccctatccaatttgggatagataaagatcatttcccaatctgttggtggcctttttatcttattgacggtgtcttttgccttgcagaagctttgcagtttcatgaggtcccatttgtcaattctcgatcttacagcacaagccattgctggtctattcaagaatttttctcctgtgcccatatcttcgaggcttttccccactttctcctctataagtttcagtgtctctggttttatgtggagttccttgatccacttagatttgaccttcatacaaggaaataggaatggatcaattcgcattcttctacataattaccacccattgtgccagcaccatttgttgaaaatgctgtctttcttccactagatggttttagctcccttgtcgaagatcaagtgaccataggtgtgtgggttcatttctgggtcttcaattctgttccattggtctacttgtctgtcactataccagtaccatgcggtttttatcacaatttcgctgtaataaagctttaggtcaggcatggtgattccaccagaggttcttttatccttaagaagagtttttgctatcctaggttttttgttattccagatgaacttgcagattgctctttctaattcgttgaagaattgagttggaattttgatggggattgcattgaatttgtagattgcttttggcaagatagccatttttacaatgttgatcctgccaatccatgagcatgggagttctttccatcttctgagatctttaatttctttcttcagagacttgaagttcttatcacacagatctttcacttccttagttagagtcatgccaagatattttatattatttgtgactattgagaagggttcaacaccacttctttatccatgtaactttgtacccattgtctttttagccttgcaaagccttttgatgtgttgtcttctactggagagtactcacctaaggagaggttacactatactcgcagctaacaattgtcaatcactccagagtttggctaggattgtgcccaactcccttctccctgctgggatttggtctggctatatacagtatatatacctgtatactccgccgaaaaagaactgagttcctatgtgtagcgcctctgcagtgttcagatgataatgcttcgtgtggtcggcatccaccaccacagactcttctgtgttcatccctaagccttgaggggtgggggttacagtaattatgttccctttgggctgagaattctgcaggttcttcatctctataccttagccagttgtgactcactgtggagaaatctttctgagctttgggtttttgtttttttattttttggttttttggtttttttggttttttcgagacagggtttctctgtatagccctggctgtcctggaactcactttgtagaccaggccggcctcgaactcagaaatctgcctgcctctgcctcccgagtgctgggattaaagccgtgcgccaccatgcccagctctttctgagtttttaatataaccactcagagctacaaactttccttctagaactgctttttctattccaaagattctcttagggtgtgtttttacttgtgattccagccatttttaagcttcctctctgactccctcactgtccaacttgttattcaaaagtatcctttttagaggctgtcaataaagtctaagaggcaagagaacctgtgttcatatccacagcactaaataatatggctaaaggggcagcacatgcctgtaactgaagctttgcagaggcagagaaagggaaatgctgggaatgtgctggcccgtccaatctagccaaatcaacaagccacaggtttagtaagagacttgtcttaaaaactgagcaagagactaagaaagagcccctggcacacatactttgacctctagcctccacatgaatggacttacacatccacatgcaagtgtacacacacatacacttgaacatatgcacacatatataccacacacatacaaaagagtataatgtatttctgtattttctgtagtttctcatactgttgatttctcattttcctcctctgtgatctgacgtgacgtaaggaattattttgattcttttatattggttaagacttgctttgccatgtagcatgttacctattaaaggaagcttccataggcggctgagaacaatgtgtatcttttataaacacatatgttggattgaatattctctatatatcctttaagtcattttgtcctatttatattttaaatttctttattgaccttcagcttgagtaatgcatctaaaaattattgtatgatattgtaattatcaactattattatttgagtgtatctgaaattttgtctccattagtgttttatgaaattaagaattctaaaatttgatgtgtatctacggttgttttgtctcttccccttcctcctcctcctcctcctcctcctcctcctcctcttctccttcttctccttcttctcctccttctccttcttcttcttcttctccttcctcctccttctcctcctttttctcctcctccatctctttttcctttttgaggcagtctttccatgtagctctgttttggaactattaatgagactggcctcaaaatcacagagattggcctcttctgtctaccgtgtgctaggtgtgtgacaccatgcctgcctcaattaatataccatcttaattagttgtccctttcattgctcacttggccttctttatgtcttctgtctaatttgttttaatgtctaccttattaaatgtaggacagctaaattcttttcagatttcactcacttgttagattgtctcattctccaacagtggctgactttacccataaggtgtggtttttggagatagtaacagggtcaatgtttttattgtggttgttgttgttaatttgtcttcatacaggcatctaggtggccaatctgtgttaattagtgagttctggctatatacatataatgttattcttcttgagagatgtttactaattcctataattttgttacttgtttctttttttagttggtgtaaaattgctttgttattgtctgttctcttagtgggtttttgttgtttcagtaatattccttcttctccttcattaaatcatttgtcgtgtggttttccaatggcttgtttgttttcttttggtttcttcatttaaaaacaaaaacaaaaaaaacccctaaaaactcaattttccttctgaatctttctttcatgttgctgacattttctgtgtaccttagagttttgctcatcagtcccactgtcatattcttctgtgttgttgactttcctccgcaaatacaagagtgaattcatctgcttgtttgtatcctctcggaattcattgatcaaataaacttgtgaagtcttcatctagatgttaacaaatctccatgtcttttgagaagccgagaattatgaccttttgggggaaatactgtttcctttgacacattcttgagtttctctgctacacttcttatacctttgggttttgtcccatctcttgaagttttatttgagtatttcatattgaatacctcagtcatttttttctccttttcctcttttgtttctgtgtgtttggagacatctcacaaggaggctggctgcaaacttactatgtagcaaagtgtgacctgaaagcccgatcctcctgcctcagccccctcggttctacaactacagttatgttccaccacacggggctaagctcctgcctcagccccctcggttctacaaccacagttatgttccaccacatggggctaagcagcttactctggagagtttggtcaccagttccagttagagtggagagagcaaactgctctaacagcaccaacatcaaaccattcaagatctagtcatactctttagttgaggtaaatgtggatgcagtaagtgaaagagataaggaaggagacagaaaagggagtagagatgtgaagaccacttaaagaggaaaaaacgaaccaagtggagatgaatatcataaagaagaataggacagggtaagaagttagaaaaaaaattttttttaaaaattccagttaaaataataaaaggtaagatcagaatacttctaaaaggataaggaataagaagaagcactctaaaaaggaaaaaaaaatcaagattacccaataatgaaaaatatgcataaacaaaatgagataattaaatgatgagaataaaagacaaattttaagactacaataaaagtgcctctaacagcttaacaatgacaagagggaaagtgaagaatccccagagttaaaaagaaggggtgtgtgtgcgaagtgctggaatggattccttctaggtcctagaaaattggagattaccagttacatgacatggtggggatcatagccctttaaactgtgggctgatgtcttgcctgtagtttaagtttcctgatgctctgttggacactctctgctggttaccagagttgttttcagattggtggcttccctttgtatatcagagtacacaggttttaagtctgtgtgctgggaaaatgctgtcaccccactgagacaatccagattgcatcccctgagatagccaatgtcttagctgtgaggtatctcccaagtgcactgggcatgtgctaagtgaccatagaagacattttctagatagatggagagcctgtcttcatgcatgcaagaacttctaagtttatcaatgagcctactagtagaatatagctctgtatttcatggctggaacttgagttctcaggctctgtggccatcaggcaaagaaatggttcatgctgctcatctacccactgatttcagactcagttctccatacaaggtttgctctcagttgatgagaactattcccagatactaatggtagggctttctttccttatgcacttacttcctgcctaaactgagctcccttccaactgaacctgtagtttatcagcagcgggcatcctgctctccatgggacatcttgtccttcctgggcctcttctactctaggtctgtggcaggccaaatccagactctcagggttatggtttttcccccgcgctttctttccctgtttatctcaggattctgttgttgtttttaatcccttcaaataagtaaataaataaataaaatttattcattgaaaatgatgtaggtgaatactgcatttgcatagttccccttccttctcatctttctaactcctcccatgttgtgctctcctaagttctccttcccaaattcgtagccttccattcttagctcaatttttagcaatagactttcaagatctttcagagcctacactctggttcccatagagtgactaaataggaattttcactgccacagacctgtgcaatggaagaccactgtctcaccaagaactgagatggacttgaaacctgtgagtgctgtgagactgtccttggggtcagtgcccaagtctcttgcctggcttagcttttgaaacggcttctgcttctttcatcttcaccatggagtcatgaaaatggaattatttggttgtatcttttaagcctgtcaccttctctgacctgtcattattctttgtgttgccttttcgatttccaaaccgtttcactccactcttcttcagattgatccccctattctgactcctttctgtttttctgtttttcttttctattagattgtgcctggggaatgtctgatccatgtttaatgacagagctcttacttataggcattcatctccattatgtctggagcagagagaaatctgcatttccaacgtggacaacaagacttccatgtggacaaaaaaaaaaaaaaggcacagacccataaactcactgtgaacattttgccccttgtaaccatccccccatccccttcctcctcctcctccttttccccttcctcctctttcttcacccttttcttcttcatcttctcctccagaaacacagagtaattttatttttattacatcaataataataactgcccattctccaaagagaacttattctaagagaaatcatcctcttagaaaaaaatcaaactatggaataaattatacctgtttactaggttattatactttctccatcgtttatgaaaatcaattgcccctgttataacatttctcataatctacaacagctctacttctgagggcttcatatttcaagttaaattatggagtcaaagagctttcttatgctctatcatgtcatacttattgttctttctttgatggcaagtagttcctactgtgtacatttaaagcatcaatatgatcttattggatacgtacagaatggtaaaattgttcactgtcatgaaacagataaatgtgtctatcagtttatataggtactttttttgacaaggccagctaagtactctttataaaatcttgactatgacaccattccattaactattgtcctcatgttacacatttgctctcacacttgccgccctatatatcccctactttacatcttttaacttacatctcatttcatttctccaaactcatgttttcaatgtagcattccctgacacatgataatcctataagaacagaaccgttaaaatgagtttgaattaacatttcacagacaatcctgtgttcttcccctttcaaaaccagaccaaccaaaccaaatctcctatatcctcagacccgaattagtcatggcaaacattgatacaaaaaatagagctcccaccttttgaagatttgggtgagatacacttcccaacctcaagcatttgaacgattagtctccacttggggacctcggggagtcataagaggtatggccttcctggataagtatgttcctgggactgggctttgagagcttcttatttgtttgttttgttttgccaaaagcaagcaagcaaacaaaaaatgcttgtcattcctgcttgttgctcaagatgtgagcgctcagttacttcttcatctcccttgttttcattccaccatcttggactctaattctctggaaccataaactagaataaactcccttttctgtaactctccttggtcacagtgttttatcacagcagcagaaaagcaactaatataccattcaaagggaataagagattgtttactttggagccaaatttgagtggccatggcttacaaacagggtactaagttaccctaattccatgttccaatgtggatactgtttcaccaagttctataatagcagaaaagtaaaagtcatgaatcaaggtaattttaaaattcattggtgggtatatcagagaagcagactgcagccaggcagataggcctcagatgttatctgataactactgagctcttccgttagttgggaataaaattttgttaatacattacaaaggttttgctgttgtagtggtagtgattttgtttggttttttttgttgttgttgttgtttatttatttttttaaaatctgcttatcaacatgttaggactgatatagagttgagcaaggagtgtctccttaaggtagaaaaatggccccaaaggagtaggtgcagagacctagagtaaaacattgtgttgagagagaggttaaattggaggtctccattggatctctctcttcaaagctaggagaacccctcagaactagaagaagtgggtaaagatggtaggagtcagagaggataaacattggctcatggagtcaactaagcagggctcacaggggctcagagagactaaaatggcatgatatttgggtaggtccttactgggtcctctgcttatgtgttatggctgtgagtttggtgtctttgtggtacctcctaatggtgggagtgcgtatgtttctgactctttcacctgctcttggggatcttttcttcctattgggctgccctttccagcctccataggcgggcttttgccttgtcttattgtatcttgctttgtcccattcttctgtactctcttgaaggccagttcttttctgaagaagaaatgcagggggagtggatatgcaggggagaagatgtagggaggagatgggaagagtgaagggaggatatattgtggtaaggatgttttaagaatctactctccaaaaaaaaaaaaaaaaaaaaaaaaagatagctccaaggaaacttgaagtaagcttcctggttcagcaacatcccttaccctccacagtttttaaagttttagtcagcctcacaacctttgcacatacagctgcttcacaggaattttctttaacaaaaaaaaaattagatattgagaagaaatcagtcatcagtctttttaaatttctttaaattaatttacggtgatgcatgagcagctcatgtatgcctcagacatatgtgtgtatttcaaatggcaatttatattaagctgaagggctaaagtgagttaactggatttcaggcaagttgcagagagattttagaatggtagactttcctcagatgtaagcgtgccagacttgtcaaagaaattagcaagctgtccaggtaaaagtgaatttcgaataaacaataattaactatcagtatagctagatgtcctgctatttattctgaagtacaaaaatagtgttgtttattacaaatacaaacgtaactgcatttttttctggcaacccatttggatgtcttttgaatttcttacacttccctctccctctccctctccctctccctctccctctccctctccctctccctctccctctccctctccctctccctctccctctccctctccctctccctctccctctccctctccccctctctctctccctccccccccccccctgtgtgtgtgtgtgttgtgcagaggtcagaggacaactggctggtagaagagggttctcttcttcactcattggtctccagggctggaaatcagatttgacagcaagtatcctttatataccaaaccatcttgcctaccttctatgaggatttcttaagtcattgtggtcatttatactctcatggactcactgacataaaactcccctttggttttggtgatttatatttttcaatctccattctggaagttgagttccagatgaagtgctgttgttgaggtctcacttttcttccacccaggagtctatgaagaaaggagcagaaatttccacaaatgggcactgctacagtagactaactgtgcgtgctttctaaactaaaaccaataatgccccacctagcttagttgcttcagaattcacttattgtgttgagattagagatggtagcataattttgagatttaagtagaaggaaaaataatttgggtgtttttgttgaatgttataataacattaaaattatttacaatgtttcaggaccacatacaattatactttgtcagctgccacctgctgttgagcataacaaaaacgttatttttttcatagttaagaaatgttgagctgggcgtggtggcacacgcctttaatcccagcactcaggaggcagaggcaggcggatttctgagttcaaggccagcatggtctacaaagtgagttccaggacagccagggctgtacagagaaaacctgtcttgaaaacacaaaaagcaaaaaacaaaacaaacaaaaaagaaatgttagcaatagtgtctgagtttgaagactgcatctgggatggattcctaagtgtggcagtctctggatggtctttccttcagtctctgtttcactctatgtccctgtatttcctttagacaagagaaaatctaggttaaaatgtttgagatgggtaggtagccacatccttaaatgggggccatgcctaacctcagatgtgttctctacaggttgtctctcccctctgttgggtatttcatctaatttcatccccatagagtcctgggagcctcttgctttcctggcatctgggactttgtggtggatatccccagttccccatcccccattgctacacacctctgttcaaactcctgacctgtcatctgaactacatctcccctgtctcctcccattttggatcctgcctcctttttcctcttcccccttctctcctcctcccaagtccctcccaccctctaccttcagtgattattttgttcccccttctaagtaggactgaagcatccacactttggtcttcctccttcttgagtttcctgtggtctgtgagttgtatcttgagtattctgagctttattcctaatatccacttatcagtgagtgcatacaatgtgtgttcttttatgactgggttacctcactcaggatgatatactctagatccatccatttgcctaagaatttcatgaagtccttgtttttaatagctgagtagtactccattgtgtaaatgtaaccacattttctatattgctgatgccaatgaagtgcttgctgacaggagcctgatatagctgtctcctgagaggttctgacagaacctgaccaatacagatgtagatgcttgtagccaaccattggattgagcattgggaccacaatagaggagttaagggatgaaccgaaggagcttaatgagtttgcaactccataggaagaacaaaaatattaaccagccagacccccagagctcccaaagactaaaacaccaaccatggttggacccatggctttagccacatatgtagcagaggatgaccttattgggcatcaggaggaggggaggcccttggtcctatgaaggcccaatgccacagtgtagggaaatgctaggacagtgaggcaggagtgggtgggtgagcaccctcatagcagcagggggaaagaagatagaatggaggtttcctgaggggaaaccaggaaaggggataacatttgaaatgtaaataaataaaataaccaataaaaaaatagccaaaaaatgttagaagacatgctgagtaggagaaaaggcatagattcagaaaccaaatctctcatatgtatattaatagtactattttatagtttttcaccatttgatctatgagaaaaaggaacataacttcaaaactagttagaactaaagagtgtaggatattccttgaacttaatgttctgttatctgacatgtttatggttttgagcttaatgcatcaaaagttctttaattgaaataagaattaaaatgtgaatctattgttagaaaaaaaaccactttatgtgagatttattaagagaggttacatgatgtttattcttttacctttaaataaattagtaataattcaggatagtgttactgctccttgccttttcacagatgcataggcagacagacagacacagacacacacacacacagacacacacacacacacacacacacacacacacacacacacacacacacatacaaacagactcaagaagtcaaaagaggttttctgtattttaagttgatgccaaaaatgtttaaccttcattttcacctattctgggactaatacatcactccattaaggtcagaaacatcaaaattccaaaacaaatcaaaatctcagagctgatttcataaagaatcttgtgaagaaaattgctttagtctcaagatcccaatactatgttattttccactccccctgccaccgtacggtcttcactgtcctgcagggtcttgtctgtagcatatattaaagaatactttcaaaatgaacaaaactgattgtacatattgtgtgtcatacatcaagacaagaatagatttggctctgtgtcctagctgctgtgacagatgtcctggcatctggtagtgtgtcctttgtacagagctgacagtcattagctggtgaccacacagaaagctaagaatcccagaagggtctctataaatcagtccagtacatcacaaaagaaacactggttgactgactttaactgtcagattgaaagctaaccagtatagttttgaataataaattagatgtaatttgatatcatatatgataaatgctgcatagaggcaattaaaatagtgagtctttattttatttttaagtgaactaattttttatcatattcagcacaatatatatttttataccaatcataaaatgatcgacatgttattaaatgaacataaataaagtcattttgtttacttgtttgttttgtttttagtagagctttaaaatcttgactcttactgtggtacaaacccaaaataagaacaatttttagatattggagttcattgtaattagtctgtacttcaatgtccctcacatcaccaaaggattttacctccatagtagttaagctaagagttgacagttctgctgcaccaaggaatgaattgcaggcacgattatttggatacatatttccttctataatcaaaactatttgacttgagtgattgtcatcattctcagcctacagggagcaggttacattcatttaagaaatggtgtgtattaagatggtctatccatggagtcattcatcaattgtttcaatgaacaatggctctgcttggagggtggcacagacattaggtgagtgtaagattctggttcctttgactgtgataattctgaaaaacattcatatcagaaaaaaagtaacttataaggtcctcttcttcaaaattgatacaataattataaatatcaaaaaaaagttcagtctcactctttgttgttctcttacaagctacctcccaaattaattttctatatttctttgggctgtatacatgattttgaaatatgtaagctgttctgaaaaccatagagtagtgtaaaaacattaaataaacaatcctacaaatagatatgctgagataggagaagcatgatttcaagaccattgtgtggtacacagctagactttgtcatatacaaacaagcagaaagtgtatatggattgtataatattggacctatttctcccattaccaaattagagagaccactggaagacagccgacaaatttctaattcctcatatatttgaatttcaattcattaagatatgttggtaatattaattttcatattaagagatattaaggaaaagtgattgttaattcctgttatttttgttgttaaaggtggaattatgtttgtgtccgtttgttgaaagattactttcttgctttttctagggtgtagtttcactccttgtgttggtgttttccatctattatcttttctagggctggatttgtggaaaggtattgtgtaaatttggttttgtcatggaatatcttcgtttcttcatctatggtaattgagagttttgctgggtatagtagcctgggttgacatttgtgttctcttagagtctgaatgacatctgcccaggatcttctagctttcatagtctctggtgagaagacttctgtaattctgatagctctgcctttatatgttacttgacctttttcccttactgcttttaaaattctttctttgtttagtgcatttggtgttttgattatgtgactggaggaaattcttttctggtccagtctatttggagttctgtaggcttcttgtatgtttgtgggcatctctttctttagggaatttttcttctataattttgttgaagatatttactggctctttaagttggaacacgtcactctctgctatacctattatccttaaatttggtcttctcattgtgtccttgatttcctggatgttttggattagctttttgctttttgtcttttctttgattgttgtgtcaatgttttctatggtatcttctgcacctgagattctctcttctatctcttgtattcagttggtgatgcttgcatctatgactccttatctctttcctaggttttcaatctccagagctgtctccctttgtgatttctttattgtttttagatcctggagggtttgtttgtttctttcacctgttcaattgtgttttcctgtaattctttaagggatttttgtgtttcctctttacggacttctacctgcttaactgtatttctttaagtgagttatttatgtccttcttaaagtcttctatcatcatcatgagaagtgattttaggtctgaatcttgcttttccggtgtgatggtgtatccaaacttgctatggtgggagaattgggttctgatgatgccaagtaaatttggtttctgttgcttatgttcttatgcttgcctcccaccatctgattatctctagtgctacctgatatatctggctatatctgactggagctcatccttcctgtgatactggttgtgtcagaactcctcagaatcaagctgtctctgtgatcctgtggttctgggatcctgaatgggtcagagctcctgggagttaagctgcctctgggaccctgagatcctggtgtgaccaagcttctgtgatcctgtgatcctcggcatgttagagcacttgggagtggagcttcctctgggtgttgtgggactggctgtgaagttcaagcccaaggtctgctcagggcactgacccagacagaccagaaggaacttgtgccactgatctggtggagttcctgtgtgcctgggtcccattggtcctagttaattcctgtgttttgacagatgttgtgtcctcctcacctctgatcctatgatcctgtgaggttttaaatcaagaaattatgtcatcttttgtgacttatttcctatacattctttcagcacactctgtccttaaatgattcttctttgaacatgagtctatggtattacaatagaattgtgtcattgaccaaacttgtgaagggtacagattttatatgtagaatgtttatcaatcaacttaatgcaggccacccacacactgctggtctcaggctttcttaactcagagcagttactgtgtttctagatcatccagatgagaggaagtgtggaacaccagagagccattttccaggtagctgatctgtgtttaatactcttctctctcatgtcttgtcagcctctttatgtctgccttggctatttgctgtttctctgtagctgtccctgtgttctgtgtgctaaactccttactccatcaactatatcttgaacacttttgctttgagatgagctggttttgtaagaaaatattaggataagaaggacaccttcaaattagctttatgctaaagagacatcaacattctcaaaaagaattctacactttttgggatatttcatgtgtcgcatattttaaacatctttcttgctttagcaagtcaaagacttctcttggaccatttttcatcctgtgttgtccaggcaatatgcacacgaatgaacagtgaaagtcactgaaaacgtaaaataactctgagcaattgagactaaatgcctctgtatagagaaaagactgccacagacaggaatgttcttggtgtctcttgggtgcactgttccatcataccatgtgtcccatatagtgctttcatatagattgtaaatactgcattattttaaggaaatcatagttaattaaggtaagtatttccaaaatggggttataacaatttactccaacatcacatgttaatatgatctgagggaattatgcatagtgtttgccttttgtttttgtttatttttccattactgtttcatagcaatgaagagacaaatgctgactctagcagaatttcctcagtctgctaagtatagctacataattgccaccttcaccagcaccattgggaaaaaaataacctacattacttgactccatatagctatattgccataactttgtagggcttacagtaatgtggtttctgtataaaactgaattgaaatctcagaattctacagagagaggaatggggacaagaaaatgaggagggaactgaagtggcttgtgtcaaagccttcttttaggaagtgtgcacttaacttctagtcagtaagctatagcagccatgttagattttctgttctcactaggccagctccttcttgatatcttaaatgctgcagcatagacacatggatggacaatgacagttctagcaaatatcattgactggaccatttcctcaccatggtacggttgtctttttagtagaagaattttttactacttaacttaaataattggcttctgattgataatacatattctttgttgtgtgtttttgtatgtatccctgtttgggagtgtgtgcttgtgcctatgcatgtatgtgctcatgagtgtagacatgtatatacccaggagtgtggagtgtttgtgtatgagcatgtgtgtgtgttcttgtgcatagagagaccagagataaatatcacacttctttctctgttgctttctaccttagtttttgtgacatgatctctcactgaacctaaaattcttgattcagctggactgtcagggcagtaggcccacatgatgcccctgtctctgctcctccagcactggaattctaggcattggatgatgctgcagctttttcaatgggtactgaagatctgaacatagatatccatgcttacatggcaactgctttactaatgaactactccccagcccactctttgtattcttgatataggctatttagcatcctttgaacaaattttcctgagttgctatatgattgttagaaggccatgatttcactgattgcctaatgataatgtgttcctcatctagaactataggtggagactcatgaggtgtggaatatctaatctctggcaacatttgtgtctgagagacaaatattcctaattaattcaactcatttagcagcctggtctttgatcaatggcatacacttgctcgttagtaatatcactgtcaaacatcactgataaatcgttacttttagaccctttgagtcctattagctttctcttgacatccagtgaatgtgtcttcttattccaggatccagactgaggaagaagcacccatttgagatataccattctcagagcaaaggacctagagcatgaggctatgtagacacacagtgcttcagagaaagcttgactagagtttataagtctcattccatcagcacaaaagtctattactcaaaattatacaagggcaagaagagcatagcaggatttccactggttggggaggcctgcaggttaaacacccaggtgaataattctcctccaactaagtggatatttgggtatcagccccgactgaatatcaagaagtgccacttcacctcatcaaacccgtcttggcctctctgtctttttagtgaatgcccattgtaaatttactaatgcatttgcgattgtacgagagatagttatatcatgttaggcatatttacaaccttgttattgtttcatgtgaacatgagatattatttgtgtcaagttgacaaggggtggattgtagtggctattcctggttgtcaacttgacaatatttggaatgaactacaatccggaattggaaggctcaccagtgacccttatttggaggcttggagatccttatctggatcttggtttgaagatcttgagccatagtggcaatggattccagaagattgaatctccgagtttaaggaacacacctttaatctgggctacacctttcatctgggtgtattgccctgttagactgtgtcatcctgtttatgaccctgcaatctccttaccaacctaaagactctcactgctcatccatatcattctctcccctacaccagaatatttggcatcatatttgagatacatttaagacactttcaagagagaagagggcatttatcattaaactgaaacttcaagcaagtaaagactacataatagaaataacacagaaaaaatacatatatacatccacaaataaaaacagagcttatacaaatgtatatttctcaaaaattgatagtatataaagaatttcctaatggtttttaggctcttgatctgagaacatatcagcactttattctgtcaaaatagcttttgaggatgggtatgatggtgcacacctttaatctcagcactcaggaggcaaaggaaggccactgtgaattcaaggccagcctgatctggacaatgaattccaggacagtcagggttaggaaaagagatcctgacctaaaaaaaaaaaattttgttctctttcttttcttttttcttctttctttctttctttctttctttctttctttctttccttctttctttcttcctttctttcttttgatgttgttgttttagtttgttgtctacttttttattggatttttatgtatttacttttcaaatgttatccccttttctggtttcctctcccatccctactccctccgcttctattagaatgttcccactaccaccttattgatctggcattcccctacactggaaaatcaagccttcacaggatcaagggcttctcctcctattgataccagacagtgccatcctctgctacatatgcagctagagccatggacctctccaagtatactctttggttggtggtttagttcctgggagctctggggcagggaggtctggttgattgatattgttgttctccctatggggttgcaagccccttcagctccttcagtcctttctccaactcctacgtcagagaccctgtgctaagtccaatggttggctgcaagcatcttctgtatttgtcaggctctggcagagcctctcaggggacagctataacaggcttctgtcagcaagtacttcttagcatctacaatagtgtttgggtttagtgtctgtctatgggttgggtccccaggtgaggcagtctctggatagcctttccttcagtctctgctccacactttgtccccgtatttcctttagacaagaaaacccttttgggttaagaattttgagatgagtgggtggtctcattccccccaatctttgctggcatctgggacttgctgatggatatccccaattccctatcccccattgctacacacctctgttcaaattcctgcccctctgtatatcatccccatctcctctcataactaatcctgcctccctttccccctcctcttcctctcttcctcccaagtcacccctaccctctacctcccatgaatattttgttcccccttctaagaagtactgaagcattcacactttcatcttccttcttcttgaacttcatgtgatctgtgaattgtatcttgggtattgggaacttttggtctaatatccacttaccagtgagtgcataccttgtggttcttttgtagctgggttacctcacttaggatgatatcctccagatccattcatttgcctaagaatttcatcaagtcattgtttttaattgctgagtagtactccattgtgtaaatgtaccaattttccattcatctgttgagggacatctgggttctttccagcttctggctattataagtaaggctgctataaacatagtggaacatgtgtctttgttatatgttgtagtatcttttgggtatatgcccaggagtggtatagctgggtcctcaggtagtcttatgtccaattttctgaggaaccaacagactgatttccagagtggttttaccagcttgcaatcccaccagcaatgaaagagtgttcctctttctccacatcctcaccagcatctgctgtaacctgagtttttggtcttagccattctcactggtgtgaggtggaatctcagggttgttttgatttgcatttccctgatgactaagaatgctgaacatttcttttcttttcttttcttttcttttcttttcttctcttctcttctcttctcttctcttctcttctcttttcttttcttttcttttctttttccttttggttttttgggacagggtttctctgtatcaccctggctgtcctggaactcactctgtagaccaggctggccttgacctcagaaatctgcctgcctctgcctcccaggtgctgggattaaaggcgtgcttctccacgcccggattgaacatttctttaggtgcttctctaccatttgggattcctcagttgagaattctttagctctgtacccgatttttaatagggttatttggttctctggagtctaacttctttttttttttttttttttttttttttttgtagtgctgagctttcttttttttttccatttttattaggtatttacctcatttacatttccaatgctataccaaaagtcccccatacccacccacccccactcccctacccacccactccccctttttggccctggcgttcccctgtactggggcatataaagtttgcgtgtccaatgggcctttctttccagtgatggccgactaggccatcttttgatacatatgcagctagagtcaagagctccggggtactggttagttcataatgttgttccacctatagggttgcagatccttttagctccttggatactttctctagctcctccattgggagccctgtgatccatccattagctgactatgagcatccacttctgtgtttgctaggccccggcatagtctcacaagagacagctacatctgggtcctttcgataaaatcttgctagtatatgcaatggtgttagcgtttggatgctgattatggggtggatccctggatatggcagtctctacatggtccatcctttcatctcagctccaaactttgtctttgtaactccttccatgtgtgttttgttcccaattctaaggaggggcatagtgtccacacttcagtcttcattcttcttgagtttcatgtgtttagcaaattgtatcttatatcttgggtatcctaggtttggggctaatatccacttatcagtgagtacatattgtgtgagttcctttgtgaatgtgttacctcactcaggatgatgccctccaggtccatccatttggctaggaatttcataaattcattctttttaatagctgagtagtactccattgtgtagatgtaccacattttctgtatccattcctctgttgaggggcatctgggttctttccagcttctggctattataaataaggctgctatgaacatagtggagcatgtgtccttcttaccagttggggcatcttctggatatatgcccaggagaggtattgctggatcctccagtagtactatgtccaattttctgaggaaccgccagactgatttccagagtggttgtacaagcctgcaatcccaccaacaatggaggagtgttcctctttctccacatccacgccagcatctgctgtcacctgaatttttgatcttagccattctgactggtgtgaggtggaatctcagggttgttttgatttgcatttccctgatgattaaagatgttgaacattttttcaggtgcttctctgccattcggtattcctcaggtgagaattctttgttcagttctgagccccattttttaatggggttatttgattttctgaagtccaccttcttgagttctttatatatgttggatattagtcccctatctgatttaggataggtaaagatcctttcccaatctgttggtggtctttttgtcttattgacggtgtcttttgccttgcagaaactttggagtttcattaggtcccatttgtcaattctcgatcttacagcacaagccattgctgttctgttcaggaatttttcccctgtgcccatatcttcaaggcttttccccactttctcctctataagtttcagtgtctctggttttatgtgaagttccttgatccacttagatttgaccttagtacaaggagataagtatggatcgattcgcattcttctacacgataacaaccagttgtgccagcaccaattgttgaaaatgctgtctttcttccactggatggttttagctcccttgtcgaagatcaagtgaccataggtgtgtgggttcatttctgggtcttcaattctattccattggtctacttgtctgtctctataccagtaccatgctgtttttatcacaattgctctgtagtaaagggagtctaacttcttgagttctttgtatgtattagataacagccctctattggatttaggattagtaaagatcttttcccaatttgttggttgccttttgtcctattaacagtgtcctttgccttagagaagttttgcgattttatgagatatcatttgtcaattgttgatcttagagcataagccattgatgttctgtttaagaaaatttcccctgtgtccatgtgttcaaggctcttccccactttctcttctattaactttagtgtatcttgttttaaatggaagtccttggtccacttggacttgtgttttgtacaaggagataagaacagatcaatgtgtattcttctacatgcttactgccagttgaaccagcaccatttgttgaaaatgcttttttccattggatggttttagctcctttgtcaaagatcaagtaaccataggtaggtgggtttcatttctggatcttcaattctattccattgatcttcctgcccatcactgtaccattactatgcagttttaaatcacaattgctctgcagtacagcttgagatcagggatggtgattccccccagaagttcttttattgtcgagaataattttcattattctgggtttttggttattccaaatgcatttgaaattgctctaactctatgaagaattgagtgggaattttatggggattccattgagtctgtagactgctttcagcaagatgtcccttgttactaaattaatgttgccaatcatgagcatgaaagatttttccatcttcttcaatttctttcttcagggatttaaagttttgccatacagatctttcacttgcttggttagtgtcacaccaacatattttatattacttgtaactattgtgaagggtgtcgtttccctaatttctttctcagcctgtttatcctttgagtagaggaaggctactgatttgtttgagttaattttataatccagccactttgttgaagttgtttatcgggtttaggagatctctggtagaattttagggatcacttaactatacgatcatatcatctgcaaatagtgatattttgacatcttccttttcaatttgtgtccctttggttttggtttttgattcaagaacaaagaatgcactaagtacaatgcaagccttatagaattagtcagtggttaagtgatcagggagatggctcagtgggcaaagacacttgttgactagagtttaatcctaggaatttacataaaattacaaagaaagaagcagctcttcaaagttgctctctgacctccagttttatgccccgcacaagtctccatatacacacttaatgcacttgtgcacacacaggcacacacacacacacatacacacacactaataaatattttagttatttttgtgagataatatcaataagaataatatttcacctgactttaatttttggcagtagaatattttgcaaagtagaccaaaaatttaattaagaagggtgctatctagaggactgatgtcctcaaaatagtggtaaggcttgcaggagctgaccatcatgttagcaagaatccagctaattttcactataaataccatgctattttagaagacactacacatatagcattgcttcacacataaaattatgtggcataatatagctaagatgtactgatattgaataaccatattaactgatagctgggattaccaggtttagaaaaacatttaaaagttatagaaataaagtatgaattgttggtgacgataaagtatgaattgttggtgacgataaagccagaaggttcctatttgaatgaagtaattcattcttatttctctttcatccctgtttttcccattgtgaattaaatttttattagttttaaatgttttcctcacttatttttttcaaaagatttactcattttatgtatgtgagtacatatgtggtattcacttcagacacaccagttcccattacagaggttataagacactatgtggttgctgggaattgaactcagaccctctagaagaacagtcagtgcgcttaacctctgagccatccctccagccctcaggtcctcacttctattccagtgtctttaggagattttactgtttcttgttgccttctttcgaatgacttgtctatatttaatttttaaaagtattttctaaaagaataattcatttttttcttggcaatttacttctttttattattttaagtatgtctgttaatgaaatgcatcatttttctttcttcataactatgtgagtttgcattttgaataagttcttatcttatatttttctttaaaatattactctctctttgacacttttcttaccacgatttgttggctatctctactgaagggtacacgggctgctctatgtaaccaaggcatccaaagtcacactaaagtaaagagaccgccacaagcctcaagccacatggtcctcaaatgagacatctctaatgaggcatgtgtttccacaaatggggaccattctcagttttcagtttaaatgtttaaaaattttaagttaaaaatccaaactttttctttctatcctaatggttatttctggatagagaaagtagaagttcaaactaaaatctatagatttttgcccataatattgcttgaaaacattaatattgaaggatatttgacagcttcagatatttcataagcaaccttatggaacaggtcatcatgcttaaaattacagttatttgtttacttagctagtatctgtgtgtgcgtgtgtgtacctgtgtgtttgtttaagagagagagacagagagagacagagagagggagggagatggaaaagtggcatggtggagcttagaggagaactcacaggaatcagtcatctccatccaccctgtggggcagacagcaagctcaaggtcaccaggcatgaacaggcacctttagcaaagccatctctttagccccagatcacaatcttgaaatggtattattcttataattaatataacttatgttttactttttggtagtatttgaatacaaggtctaatcagaaagtaagaaatactttccccctaacctgaatatgttatcagtatagtatttagttaaattactcacatatgagtgtaattactgtaactgaaccaggtcattattataacaataaatttgaagtggtcatattcatcagtagtatttgcctatctttttggctgtttgtttactcagttgatactgacagccaatagccctaaggttttttttttttttcattttttcttatactgctttttatttatatttaattatatttaatatttttcttttatagtcaagttgttatccccctcccattctgccctcagacagttcctcatcccatttctcctccccaccctcccacccccatctctaaggggtctccacaccccacactccccccaacacccctgccaggtctctccattccctgcagccccaggtctctcaagagctaggggctcttttctcactgaggccagaccaggaagtcctacttgtacatgtgtccaggaccttggatgagctagtatatgctgcctggttggtggctcagtgtctgagagatctcaagagcctaggcttattgagcctgctggtcttcctatggggtcgccctcctcctcaacttctcccagccttttcctaattcaaccacaggggtccctgacttcagacaattggttgggtgtaagtttctgcttctgtctcagtcagctgctttttgggcctctctgagggtagacatgctaggctcctggctgtaagcacaccataatactgttaggtcttgtgggttgctcatttttactgaataggaaggcttgaagtagccacttctgttctgtccacacaattttatgaaaaatggaccctgttgttgcaccctttcagaaagactgaaattgtatgcaattagtatgtatttaaattatgctgcaatccaaaatttaaatatttgcatacaaaagataatgctaaacctatgaaacagcaaagaaatacaaacgagaaaaaacaaacaaacaaatgcccttatatatgtgtttacgtttgttctcagtaaagagagagagagagagagagagagacagagagagagagagagagagagagagagagagagagagagagagagagagagagagagagagaagcaaagatctatgtgctcggcaggattatcaagcactgcacactggtgccttggtcacagacagattgcaactcaaagctggctccatgagtggagcttagaagtcccaatacttattcatgtatccatcctaaactcatggcacaacacaggacatgtgaggtaatggtggcatccacagacatcttctgctgccagtcccatagatcatggcagcctttcaattatgcttgtatcataacactcgatgatgataacagcagctctgtcaagagtttgtggattttctataatattctttttaatcatattttagagtatatttctactaataaagatgtttaaggtaataaaagagcaggccacactgtgctgatagcagcttcatacgtctcgtgaatattggactctttgatgcattaatgttgagtgatttgtctatactacgtaagtccatttaagtattctattatgtttccacattaaaatcatctactgatttttttctcaatatatactcatgcttttaagtgacataaggctgtaaatagttcagttcacttccaaatatcagatcataagtattatctacacatcaaataagtagaatcagcactagaattgatagaataaaatcagagatggtggtgcaagaccacaatccaaacaattaggaaagtataacatgataattgcaatttcaaagccagtatgagccatggagcaagttccactccacctttggtgcatgctgagatccaaactcaacagtcataaacaaaccaaaagatagggtttatagaataagagaaaaaaaaggagaagaacaaataaacacactaagatagaattcaatgcacatttagactcaaaaccatcaagtaggtgagagggatacctagacaggatgatctgaatgaatgagtgagataaaatacagtgattccattgcattgagagggagcccacctctggcactattaattacatactgctgttcttgcagacaggaggctagcataacattcatttgagtggcttcatgcagcagctaatgaaaatacatgcagaatcccacagccaaacattaggtaaaccttggaatcttgtcaaagagggagaggaagagccagggagatccagaacacaagagatcctacagaatcaactaacctggtcgcatagcaactcacagatactgaaccaccaacaagacatcatgcatgggatgacccgctattcatatgtaagagcttaacagcttggtcctcatgtgagatctctatcagcaagagcaggggctctctcagactctattggttgactttggatcccttccccctaattggacagctttgtctagtgtcaatagaagaagatatgtctagtcttatacatcttaatatgccaaggttgggggaggagggttgatatcaatgggaggtttcactttctctgagaagaaatggaaaggtgaatggggggagggagaacactgggaggagaggaagaggaagctgtagttttctattagagacctagacatcaaagctgcaactaattgtattgagcttgctgactttaatgagctaacagactattaaaacagagtctgtaccaggcagaacttgagctctgggaagtttgggcacagatatatgtgtgaaatttgagttcttattaagtatacctttaaaaggatgcatcttggatttggccttaatatctgcaggactgcagactctcccatgtatcagaattgtttcggtgaagaaaagtacattgttccttgaactaagaacatttctactacctcctgtgcacatagcttcctttccagtttagtttatgtccatctcagccatattaatggcatttgtcccaccttggattgtcattgtcaaagagactctactagagctaatgtagttctcaggccatagatctattcacccacatctcagggtgtacacaggcataagtagtgtagcaatgactgtctttttatgtgattctgattgactgcaacaaagttaaactgctttgtccttacttgttaagcctcaatggctgcattttagatctcagagacaaggttccggttttttttttttttttggttgtgtgtgtgtgtgtgtgtgtgtgtgtgtgtgtgtgtgtgtgtgtgtgatacctttgaattgattgatctatcagtgggcatcgtcttcatcatacaggtacatctcagagtgaacagtttatcagtgatgtggcgacaattatcaaaatgtactcagagcaatactgaaccagtgtttaaactcacagcctagttcctctattggtaaagcacaatcctaaacatacaagaatgcttggccaaagctcaacatagagaatgaatttgtgtaaacattttttttattttttccatttaaattttggaggagaaaaaaatttaagtcttctggcccacttttctagatgagtatttattgatattgataacttttaatgttctccacgtttctggttcttccctccttaggaatctcacatgaagagtaaagagtaattaaaatgtgcaggatgacaagatggagcaaacagtgcttgtaccaccaggacctgacagcttcaacttcttcaccagagaatcccttgcagctattgaaaggcgcattgcagaagagaaggctaagaatcccaagccagacaaaaaagatgatgatgaaaatggcccaaagccaaacagtgacttggaagctgggaagaaccttccatttatctatggagacattcctccagagatggtgtcggagcctctggaggacctggacccctactatatcaataagaaggtaagtgtttttatcaggtatactttgccactaactacatttacattggactgttacagaaacctcacatactaaagtagcacaggcttaacaatccctctgcttaagaaatgagaccagattggtgggtggtttatgcatattcataagaagtaaataaattatattataccatatggagctgccccaaatgccccatgacagaaatctaaatatacgtataagattttgtaggcagctaatttaacaaattaaaaaaaaactctcactgaagattacaaggactaagcaattttaagtactttttttgtaccttgaaatgttgaagtcaccctgagttaccaaagacctaaaaatatattgtgtgaatttctcatggaaggaagttttacaacacaagaggtttttatttctatgccatattataaataataaataacttcctaagtaatcgtttccacatggaatacagttatgcaatatacatgggtattttgtgttattgttgttttgttgcttgtttttaggggaaaatgatttcttttggcttatagttcacatatataggattgttgggggcatattgcaattcaaccatttcagcacatatatgtatgtatgtacgtatatgtatatatgcatatatatcataaccatattatgtctacaaagactctgtttctaagattgatacagtttcatactggcttgaactttttgaagacactatctagccaattacacttgccagttgttaaagttgggggaatatataaccaagcaagtcaagtgtttcattgaccacgaatcctaaaatactccaaacttttagggacactgtactgttagcccagttcctgtcaggacaaaagctgccatatgtggtagcttggggtgagagaaatgtcttagttagagtttctattcttgggataaaacatccacacccaagacaacttgaaggtagggaagatttatttcagcttaccgttctacatcacaatccattatgaagaaagctggggcagaaaatcaaggcaggaacctggaatcaggaactgaagcaggagcgggggaagagcacgctcactggcttgcttggcctattttttttatagcacctgggaccaccctcctgggggtggcactacaacaacaggctaggccatgccacatcaatcatcagtcaagtaaaggcacttcaggcttgcttccaggccaatctagagaaggggcattttcccaactgaggttcccttttccaaaatgactgtagcttatgtcaaggtgacataaaagtagtcaatttaggagattgttcattctataattgctatttgtcagaagtgaatatgaggaaattacaaaataaagatccagatgtgttccacatagaaattggttcatctcatggattgtgtttatttacttacaattctcgggggggcatgcagtgtttcagaggctcctatggtccaggttagaaagccttttcctcatcaggtatttactcccccaaacatggcacctccaatttacagaccaaatccaccacctaatagttactatttatttatatagaaacaaagataaataaaagtgatttattaatgataaaaattactaagaggaggccctgtcaaaatacaaaattgaaatccagtatcccagcccctatacgatgtccttatttataaatctataaaggaataaaagaccaaatggaaacactttaaaaagcatagtaattcactatgtggtaaccagactgtcatagtgttatattgctatgaaaagacaccatgaccacagcaactcttataaaaagaaagcatttatttggagcttgctcacattcagaggtttagaccattatcattatggctgggagcatggcagcactcaggcagacatggtgctgaagaagtagctaagagttttatatctggacctacaggcagcaggaagagagaaccactgggcctggcctagcctgagctttggaaacctcaaagccctcttccatggcacccttcctccatcaaagccataactatgtcaagccacaccttctaatccttctcaagtaataccactcccctgattactgagcattcaattatttcagtttatgagagccattcttactcaaatcaccatacaaactatcaaactatcagatcctttttttttttttttttttttttttgtcttttaaattaaagagcttccagactaccaggcctggttgtacacacctttaatcccagcaattaggaggcagagggaggagagtcactgtgagtttaaggctagcctgatttaaatagcaagttccaggccatccaaagttaatagtaagactctgtcttaaaaaaataaaataaaatcaatcaaccaatcaacaaataataaaaataaaagcaaaagaatatgttttcaaaaacaaagagcttttaaattgatgaaagatactgttttgaggggtggttctacccatgatggtccctgactgggttattttgaatttaaaattttaaaaacaatggcataaaggaaaatggaaaacaaaatcacataacccagtgacaaatacttaaagcttttacaaaaagctcaaggtgtaagcccagctctgatgacttggcagccagatcagctttcctacccctgcccccaacctgcctttgacatcagctgtccttatcttcaaatgcacattgtgtgaagaaggaaaaacattcagacattttggaacaaatcacattgtgcaagcagaacaacctgataggagagatattgttatcaggaaggagtttttccaatgaaaggcatgtccttcattttcacattattcttccaccaaacagatctccagatgtgagccactggatagcagcgcaggtagacatttctagagctaagatttttttgtttgtttgtttgttttttgtttgttttttaaacagacacgctatgtgcttcattctttagtttttgagacagcaattccctatgtatcccaggctggccttgaactcacactcctcctttctacaggtgcagggattgtaggaatgtgtcagcttgtccagcatagagtactttagaacacatggccaagccacattcagaatgttgccttctttcatagtgctctgttttaaaaccttctttaggttgcctgtatattatgttactggtcttaaattttctctcataaatagattgttttcttatggtataatcttcataaacattaagttttctctgatttacattgaagttgcttagtaaaaatctcagtcttttaaaagaatatttcttctaatatttgatcactaattttgttttagtgactactatgtagccttcatatatatatgaatgagagagagagaatgggagacatagagggaaggagggtagaagggaggggaaaagtgaatgagttgaagcagccaaggggtctactggtcaaaaatcattaagggtgagattctccatgactgaaaaaaactgtgaatatggatttgttttgaagattgagttgaaatatgcaaacatcaatggcttcccttagattgaacttatttaatgagttatttttatataaatcatcatgctcatttattagcaatatgtaaaatgattttaaaaagccaaatctctaagaccctttagcctgctgtaacatcacatccaacatttggaattataatgaaaagaaatggattggcttaaacttatggagaccagagagtccaagattgaggaactaaaccatcacatggtggaagggtttaaggcaaggaggagcaagaagggtctattatcttaccctggaaaggcaccaattcattcttgaaggcaaagccttcattaaccaataacctgttaaatattcccaccttgtattatggaataattcaatttatgagttttgggaagggcaaatattgaacaatagcattgaccatgacaatggttacagaatgtattctttatggtatacctcaggctaaagtgcaggtgcaccatatgtgcagagtgctgtgtgaaggcactgtagcatgccagggctctctgtgttctttttataaagataaccaatgccattgggctagtggaaatgtcactgacagtggtatatgttatcgtgacatatgcccacatatttttatcctctttgacccagtttctgatctgcaaattatgtaataatcaaatgttctactaaaattattaaagtgatttaagctgtgatcagagatcctttattagcaatgaaagaaacagtaacaaccgtgaagtggaaaggagatagcaaagttgcaatgcactgagtttccatctcatggttgctgtcctcaggggacctttaaggtatgagtcgaagctgaatactttctatttgttccttcactttgtgtaacaaggacatttcagtttgtgagtacaattttttcttatgggactttttttttgtcggtttcataacttgagctactactgtaccatttgcctatacattcagcttcacccactctttgttcagaaaacgtctcccctcttatgttcttaaaattacagatgactcattagtgccatgggaagaaatatccttttgacaatgtggcatttgactagaaatagaaagatattctagatctgcagtgaaaatagaagcttgttatattttggaatatagaaatgtattcatttaaaaactgtactgtgtccttgaatgtttcctaacattaaaaacaacctattgtctccaccaacagacctaaaatgatggatactatctctctactggggttcacataagaggcttccaatttatatctcatctgtcatcacaagagtgtctatgattgaggtaccaagttcttattttgttttcagttagggtgaggaaggttttattcttctaggaaaatcagcccttagcctctgcctcagaaaatgtcccaggacaaataaattacctttgacatttagtttattctagtcatactagttaagagaccctttggataaaagtaaccaaaactcagcaaaaactactttgaaggagagacagaagaagaagaggaagaagaagaagaagaagaagaagaagaagaagaagaagaagaagaagaagaagaagaagaagaagaagaagaagaagaagaagaagaagaagaagaagagggaggaggaggaggaggaggaagaggaggagaagaaggagaagaaggagaagaagaagaagaaaagcaaacttatgggccaagtctagtttgaataacaaaagacgtggaatctaaactgacaccatcaggacagcccatggacagtgtaggctctgctttgctctgtgatggcttcctgcataggtgggctcacccacacatggcaaaaatagtccccagttacagtaagttaacatctacagcagagtaaccttgaggataggtgcatgcgttttccaaactctgagcaagaattctggaccaggtaagggtcacatgctgaatcacgtcctaaagaatgaggcaaatgtatttaggccacaaggacagacagtggagcagagatgttctccacaattagaagatagaaacttctagagaagttattccagataagtaaaaatgaacaactgctacctccctatatattatgcacttgcattttaaattcatgttggtaccttacaggttctttcaagacagttttggtctcaacttcagaataataaaactgaggttaaatagtttgtctaagattgcaaaaactaataagtaagctactggcatgttgccaaacccctgctttgtaaaaacaaatcttccatgctcagtatcagcgtctaatacaattcaactacacttaaaacttaaatggtgggcttctgaaaggaaatgacatctgaatttagatgcgaagacaagcttctgtaaaaaggaaaacatagaagatgctaacagagcatctggcctgtcatatgaggaggaggttagattctctagttgcagggtatacaatagatcttctatcatatgatggccttaagttagtgaagggcatcatttaggagatcccaaaaaaatgtcaattcaaataatgccatagcattccctccatctgccctgctttctcttccaacctgagaaaacatagatgtgacagaatcttccctctgtatacagtgcgctcttttgtatgagagtagatccaaagttaaaacttaagttaattgtgaagtactgatatctataatttttgctatttttgcacaatactgtttaacttttaaattatctcccaaagtttataaatgtgacaataatgactctcagatacccattccctctttccactctctggtcatttcagcttgtaatcagtggcttaaagaccttacccattaacttggtggaggctgtgttagattaattgaggttagcctgctgccgagaagcaaatgctgtgaggtaagctgtgaggagacttgtcatccctcgtgagccaaagaggaaaatctttgtttgaagccatgaatataaaagaggaggggaggaggaaaggagaccatttaaagattctcagagagggaaaaagacttagacgcatggattggacaagaatgaaaaatgttcacggagatggagactgacaggaatctctgactacctgaagcatcagactttgaagcatcgctatgttggtacatgactctcattctgccctcccacccccgccccttactgcggaacctctggaaatgtgccccactccctgatagggttagggcagtttctcagtctgaccaccagagtcttacagagtagagatgcgcacctgagcccaacttagagccatgttactatttccagactggcacttccttctacatagtgatacagttccaaccaccattaattcataattatatttctttaggaattacataaggcctgggaatagttcactggaggagtgcttacttagcacttgttgggcctggcttggatcccagcacctggggaagaaagttacattgagtttgagacaaaaaacaaaaacaaaacaaataaaaccaaccaaccaaacaaacaaaaaaaaaaacaataaaaaagaacatatttatttactctcttctgtgaaactgcattaaaattccaaaggaattgactgccagatacatttttagaaggcagatgatgtgtgaattaaaaatcccctgtgatttaaagttccaggcaaagaatatatttagtctgcttataacacctactatttatttggtttttcttttgagaaaaggttttattccagcactcaatcttgcctcaaactcacaacactcctgccttagctttgggctgctagctttacgggagtgaggctccacatctggatgtacagcttttaaaatatttctagttatccagctcactttttgtttttgtttttgtttttttgttttttggttttttttttgagacagggtttctctgtgtagccctgggtgcctgggtgtcctggaactcactctgtagaccaggctggcctcaaactcagaaatccacctgcctctgccacccaagtactgggattaaaggcgtgcgccaccatgcctggcatccagctcactttcacatgttatctctgacttttgcctgtagctaacttttagagagggaatggagtacatttcaatgtaatctctgttatgtcacagctttctggcaatgtctagatgaaatgcaaaattagaacttaagttataactgtatactttttttttttttaaatgggctcttgattgaaataaagtttccaccaatggctgtatattcagaaaacaaaagtgcaaatacaccacttgtcaaatccaagtttcatccctgacagtaatcgcagcagccagtcatgttctatgaagttactaggtcacagcaagcagaagggcccagtccacatatcctgtataaccattgagaactggcgaatcactactgattaatcgctaaccagcaaaactgttgcttcacaaaactcttgctcttaacttgagacaaatctgctgttatttttccctgaggtttgagttgtttgtttgtttctttgtttctttgtttctttgttttggcaggaggggaggggtcattaaaactgttgattaccagggatgcttatcggttttcaatcaaaccaccttatgcttttctttaacacctgaaataatatttttccatcaagggttgctgaagagtgtgacttggtctgtcaatgagtggttgaaagtcaggtcaaatctgttcagcacagtagatagacatcagctagtatggctacgggtacctaaaatagaataagaccagattgagaagactggagacattgtgaaaaaaatcacataaaatacctcagtgatttttttaatatgatgttatcctcaaatgttgtcgagttacagaaaacaccagtaagacgcacttcacctcttccttggcttttcatgtagtacttagacaatcagaaatccttggttaaagtacaggtttgtctttacattatgttcctattggttagttctaagctatctacacctccatatgttcgtcttctagattcatctgtgctactatctgtagctgagcattcctttgcaagtgcccggatttagatatgtgtgttttcttttcagaatgcagcacaaagctttgcaaatatagtgctaatcagtcaccttttacacctaaattagagcatatgacttcatttttgtcatataaatattacaaagacattttttaacacacacacacacacacacacacacacacacacacacacacacacacacacacacacgccggaaaagtcctaaggcctaagtaggaatacttagttcccataaatacatctataaaccagtttttatacataaaggttatatctgaatttacccattgtgccttgtttaaaaacttgttccaaggaagaaaattattctggtattaaaatgttctcatcaagtgcttatatttctactccattgcagtaatgagattaaagcatttatcccccccccccctataataaatgaatcaagggataggtttagtagaaggtttagtaccttctactaaaaggtacactcttggtgcttgccactgggcaatttgcacatgtaaatagagaagggggatgtctgggtggttggtagctttggagttaatcctttgttttaagtgaacattgatccccttggatccttttctctttcctccatgcagtgccttcagtgcggtatttctgagtggcactgcagttgtaacctgacttctcaactatagtaaagttgtgaggaacatagtagatactatcgggtaaaaaaataaatagcagaccagttggaaaaagtaaacttaagttctaaatcaaatttaatgacttcataacgactacaatcctacatataatgaggtgctatctaagtctgaatactgtctctattgcaaagctattgaagctaaacccataaacacttcagagtgatgtgtaggaagctcgcatttgcatgtatgtttcaatccaaaaggaatatatctatcattgggtaagaaacaatgaaagtggttctgcaaggctggcactattgttacagtccttggcaggggtgggaagaaatcttatgaaaaattaaagtacagaaagcatgctgtgcagtcaaagctcatcagtctcctgtttcaacccacctacattgcacatgaagttctttatgcaacatgtttttacaaattatttgcatttaataaatagatgtatcaaatatatcataaaggacagaaacctttgaaaatttaagaattatgtcaaggtgttcaaccactggtcgtccaaattttcaattattaatcaggactggggttgccgagctattaccaacactgctttgctgagttccagtgcctaaagttccctcctggttctctcttgctaagtttcagagtctcagctttggcctcactaataattcctgttttgtccctgcttcctcttttctcttttctattttctgtcaagtgttcctgtagtggtgactttgctactgccataacaaaactccatgactaaggaaacctgaaaaggaaagcatttattgtaggggttcgcatgagtccatgaccatcatgggggaaagtatgacagcagtcaggcaggcaggccagcatggtgctggaacagtggctgagagcttgtactgtatccatgaataagacacacacacacacacacacacacacacacacacacacacacacagagagagagagagagagacagagagagacagagagagagagagacagagacagagacagagacagagacagagtcagacagagacagagacagacagagagaacacaaactgaggttggtttggacttttggaacctcaaaacctacatctgttaacacatgacctccaacaaagtccttcccaaacagttttgacatccgaggcccacatattgaaatacacatgcctactggggctattctcattcaaaccactacagcttcctttactcatcatgtaaacagaaggttgccaaggaacctgtgtcattgcgattctgagtatagccagtttcaatgggatgagcctgggaatttccctgcccactgaactactactgaccctcccaagactcatcctccagcctgtaaaagatgctacagtaaccctgaaactcgtttgtcatctctatcaccatagtcatttcaaaatcggtctatagtaatatatgaaattagattcttaggtgaagagtatatattctttctagttacaatttttttaactacagttcctactctagcccccaatatttctatcacatttgactggctgtgtttctggataaggaggtacaggcaagaactttgaaaaaaatcattggtcctaagctttgctcgttctgtgtccttggatgctcacattccttaaggcaaggatatttcccttacccaagtccattcatgttttatgatgctttcacaaggtcgtttagggacaaaaatattgaatgaatcagagagaataacgggaactatgtaccaaccacaagcgattgccactggagtgcgttcctccctcacagacatcttaaggtcgtctcccttgtgctggatgcacccattcagtgatgattagatgacatgtggtcaaaggtcaggcttagagaagcagcacttttcccacccaggtgatgtggtctctgtttatctatgacacagtgacattaattgctaagtaactgaaatgaaatgggatagcccagcctacagtctcttgttttattttatcatcattttcaacagactcaaaattgcattacaactttctaaacacaggttttactctcagtagttggattttgtctgtgtgtgtgtgtgtgtgtgtgtgtgtgtcacagctgagaaaactaacagatccccactacttaggggatattttcatagtacatttagagcttttctgtttgaatcccagagagaaagggctattttcaaacagaaactctgtgttcaaggattgctccatttgaaaatctcaaggtcaaagtgaaaaataaaatgaaaacatgttagtgacctaaagtttaaatgtgaagaagaaatgctatcaaaaaatgctcaggagaaaacaaacggcacgttatagagtgtgctaatcatagcagggtccatagagctatgcatcaaaaggctaaaacatgggagttttaaggaatagatgcataaacatattaaaagctgtgctggggacagcattcggtggtggatcccttgtataacactctgggttggttatagattatacacgcatgtgcacacacaccctgagctgggaacatgaccctatggtaaaacacttgcttagcatgcaggaggccttaattgcattccccatacttaggaattagaatagagtcactaaaaaaaaaataaactcaaaccttagcattctctctatttagtgtgttattcacagagatgtcagcttcatccatctcggcctaggctgagtatcagtaaatgacttagaagactgtgtcagactaagccgtgttgctccattgcatggtgagtcttttccctctgcacactgatctttgctgtgtagtaagtctgactcttgtcgtagatgctcttcatatttatgggtcctgagttctccagggaccttgagcttttcctccacacaggggagagtcaattgggattagaaaccaagtgtcagctacctcaggaatcccaatacagaatttagcactcctggtcaacaagatactctccagggagcctgtcctgcagaaatcaatgactggatagtatccatccaataggtgcgggctagatggaagctgcttttgttctctattaccagcggagcgatgatgtttattttataagcacgggtagtgaaggaatatttatttttaaaacattcggttctgttgaatgattttgcatcctgcaggattcattttcctgatacttcatctaggcaaacaaatcattatgagaaggaattcttgtgaatgttaggatgaaaatgaaatgtgtccccatttcaaactttggcttaagtaagacacttaaaatattaaggaaatgaggacattctcgtggctgatgcacaaactcaggtaagtggttggtggaaggtaagagccactgtaagttcatctctgggacataagtgaccagagagagctgtactcatggtacatatcaaaagtgattgtcctgggcatctgaattggaattattcactgcaaccctattggaaaataggaagtctgttttatacttatgtgtttattcctattttacagacttttatagtattgaataaagggaaggccatcttccggttcagtgccacctccgccctgtacattttaacacccttcaatcctcttaggaaaatagctattaagattttggtacactcatatccttttgcaagtggttggtgttaactgtttgtattcattgtgtcttctatgtattattaagttaaaaaaatgtgcatatactctatagctttcatctaatatcttttgttggtcaaggaaatctctttttgtttttgtagtgctgagaattgaacccggggcctcagaaatgtcagggaaacaccctaccattaaggcaccccttcagccctgaaccctagtctacattccaggatcccagccagggaacggtgccagccacagtagatgggtctttccgtctgaattaacaaagcagtcacaataattccccagagacatgcaatgaggcccgtctgttagagtattctagattctgtcagattgacaactaacactagctttcatatatgtattgaatcccctaagttttgtgcaagatcgccatgtttcttgtcaacatctttgtacagagttgcataaaattttcagtaaaattaatcacacacaaaaaaactattttcaaagtaaaatacagattagacacagatatgatgtatgccattttaaagacctttggggggatcatgtaagcttataagaatctcaagacacttcaggcttagctagttagtcaacttttctgatcagttttatgaagttggctgaatatcagatttaagccacagaatatatgggtctgttgtatagcattgtctctttagtgcagattcaggaaagaagctaaaactgttctaaaaggttgaagggagtttgcctctgtggtcgcctttgggtttaaaggtttctgcaactcagttccggcgtggatattgatcagtgaaaaaccttcatttatgactgagtctcttttggtggccttggtcacaattagtataaaaatactattgtaagtatcataaggtcatactagcaagattttttttaaaatcacaaattctaaaatctaaagagttgtccaaacacaaggctctgggtttgatttgttccctggcactacaaaaaacaatgactaaagaagaaacgttcatttaaaaaaaataaaacaaacaaacaaaaaaacagcactacaccaacaaaaggagatacaaaacaaaacaagaaagtaaaataataactaacaaaaacaataaaagcaacaacaaaaaataaaggcccttctttttgagtccagttgatcacaagtaaatcgttagtcctattaatagtctatcatcgaacactaaagatgtaccttataaaaagtgcctatgctcactcaaaagcttttccatcttagtttgcatttctagtgccatcatgtggtcatttgatgaaatctttccctacacctaatggaaatgttttgtgtggttagtcgagtgtagtagaaagaatacctgctactaagacagaaaacacatattttaagatcctgcggatcatctgttttctccaggacgtagcggttatttctggcatttctaaaacactttttacagccactttcaatggccctattacctgttcttctctgacctaccactcagtgcgtacacatttctccagcggcattgcttccgagcatttcatactcactgattactttaatctacgcgaccaataacaaagtctgtccagcttaacatccgggtgttgaatgtaggtggagccagagggtggttgaaggcacagaaaggataagtgaccgattggcccaagaacaggtgtcactgtggaatttcaattacatcccagtggcgatgagccctgaccttcacacatctccccttccatcttctcatgctgttgtgcttacataaactcactcaaaacacggctacttagcactctgcaccacgctctgctttgaccgtgacgtcaaaattctatccattaacgacgagatctcggtattgcagaaatgcaccttcctcgaaaatttactattctaattgtgcaattaattcacatccatgctttctgctcaacaatgttgcaaataaaatttaaatacattttaaaagagagaaatgcatccaccacccccccaaccacctccacttcccgcttagaaaaagcaccttggaaggtaatcatgctggtataaaaacaaccgcaatagcaacttgtattcttatgtccttccatactctcatttactctgaaattgtggaggtcacactatgatgcatatttagcatcatatgaaatttgagtgaaatgtaaggagattaggtagtatttgtgcacatgtttacccatgagtcctgttgaactgaggtctgggtttttcagtgttactttggttttttttaatacaatgcatgaactttcttttgtatgttccttaacactaacctacattattcagcatgttaatcatgtgcactattttgacaaactgtgtatttatgacaatgagtaaccctcccgactggacaaagaatgtggagtaagtttattttaaatgtttgttcctgtacatatatttgagatttttgaaactatttctcaatgtagcctttaaattaaactggagaacatgtgtgataaagccatttatagatgcagctttaaaataagacagcagtcattgggtcccaggcttgctggtgtcagatctaggatttctctttactcatataattgatacaaagtctactagaaaactcaagggttttctttcaggatccagtatttgaaaagaaaattgatggaagtaactacagaaatatagacaatatatgctggactttttattccagataacctccacattaattgggctaattaagaatcaaaatgattaagctgtaaatgctacattaagaaccacagtgtaggggaatgccagggcaagaaagagggtgtggctgggttggtgagcagggggagaggggaggggataggggattttcagaggggaaagcaggaaagtggacaacatttgaaatgtaaataaagtaaatatctaataataataaagaaccatattgagtctcttgatcactgggatttatccaggtttatatctattttaagtatgatcatcaagaacttaactaaagtttcaggattataaaacttggaaattcagtttatggaaggctaaaatgtctacagtggaaattcaggaagcattctcaagggctggaagcattactaatgacactggccagctcttgatgccccagtagaaggagaggcagttatgtaactggagctctggatgtttcaatcgcagctttcgtgcctgcccatgttctgacacctagaactctctggtttctgacctttcaccccccacagtgtttcccaaagcctcttcttctagtgcttctaaatggaaggtcagattctgggaaaaaaacaaaacaaaacaaacaaacaaaaaaaaaaccataagaaactatgtatgtgatggattctgaaactattcaaactcacatgtttcctttgttggtatccatgttttccgaggacaacatgctaagaagctgatgtactctgaatgaaattacaaagtccttaattgatttggctacttgtttctaggtacaccttcacaggaatatatacttttgaatcactaataaaaattattgcaaggggcttctgtttagaagattttactttccttcgcgacccatggaactggctggacttcactgtcattacattcgcgtaagtgccttctcttttggaaacctgagagaatacagtttggtttgctgtctatgctcaggtttttaaacaacatgctcgagtggtaaaacatcctggcatgatttgggcacctgaactccgtgtcttaacatttacctcaaaagggtaaacccattccactgctccatgagaatagtaatagcaattttaataacacttaaaacatcagaaaattttgtagccaggcaggtgtggcgaacacctttaatcccagcactcaggagacagaggcaggtgaatttctaagttcaaggtcagcctgctttacagagtgagttccaggacagccagggctatagagagaaatcctgtctcaccccctaccccccaaaaagaaagaaaatattgtatattctggtctcctcacacaaatacttgtaatactgaagcataacttatttatctcctgagctggaggatggttttgaaagttcacacagcaaaatttgcactggtcccacaaacttgcactgaacttttcactgatagtcctgttgtgcctcaccccgtggcttcttttgttacctttatagaatagtagaaagattgtagccatgctggaaataagatcctaaggtgaaaaagacatttgatacatcataaatcaaataaatggttatttcttgagcagacataccttgcctctatttcccttctgattcaatttgtattcctctctctctctctctctctctctctctctctctctctctctctctctctctctctctctctcgctctctctctctctctctctccctccctccctccctccctccctccctccctccctccctctccctccctccctccctccctctcttgtttttaggctcttctttattttactcaacacaaagcttttcaatatacactgagttgtatattcagtccctactttcctgtcttctcccttttgctagtgataaatcacgcccctccacaccaggaagagatgagaataataggtagctattcaagaatagtcaaaagtaggtatggaggacagtggattaataccacttatcccatctgggtagctgactttctgggcatcagcaggagtgaagggatacacaggacaggagatggtactgatcggctccaaagtgcaaatcctggatgtctgaccttaactgtataactttatgtctgattggaatatgaaaggttcccatgcgtttcagccacaactttcaggacagtatttgagacttctcactcttgagactaaaatagaagtgatgacagagtggccttgggccatgtaagatgctattgttaatttatttgttcaacttctattcaaacgttattttaggaaatgcgagtcttgaatagtggggcaagaggcaatcttttataatcgatagttctgttggatgatttcctatatcatttcttttctagctgaaaccctagcctgtggatttggttccagttaactaattaatttagttaattttcccaattcacatatagtgtttctaccttgcctaccaaatggcatcaagattgtatattttatgaaatttacatttagttgtggtatttatcatgaaaaataagactacattgatgaagtctaaattttaatttgcaacttacactatttaaaagcagcaaagatcttcataaaatatcagataaatgctagtgtcttttcaatcttaaatcatttacaggtaagtaacaaagtttgtatacctaggtaatttttagctattcacacatttagaggattgagtctaaagaccattcccacatttctaattagaagcaattactataaagcataaggtaattgtacctacaaaactttctttgcttcctgttattgtgtctgtgtgtgaactcctcattacagatatgtgacggagtttgtggacctgggcaatgtctcagcattgagaacattcagagttcttcgagcattgaaaactatttcagtcattccaggtgagagctatatgaaagcatgtaggttaatcagtgagttaactctgagactttttcaactgagcctccttgcttgccattcatcattacagaaagctgagaatcataagactgagtacttcctataagattactctctctggaggttttatcaacctttgtgtttaatttgtggacactctgatattcaacccaaccaacttttactatataatgtatttttgtgccggacagtggtggtatacacctttaattccagcacttgggaggcagaggcaggcggatttctgagttcgaggccagcctggtctacagagtgagtttcaagacagccagggctacacagagaaaccctgtctcgagaaaaaaaaagtatttttgtaatgtcaatgttataaagaatttgttccctattatatgtataaatttttcatttttattcattatacccattgatgcctatgcaaaaacaatcccattctttgctaacaaaagaccaattataagacttaaagaataacctacaacagcatgaatgctatgttcttaaatgaagctacactctcgaactttttataggacaatgtccctgccatgggccacattttgggacttacacgtttatttagtgtctccgagagacttgtgtgaaggacttatttaatatttcagatgcttgatacacttctttctctcaggcctgaagaccatcgtgggggccctgatccagtcggtgaagaagctgtctgacgtcatgatactcactgtgttctgtctcagtgtgttcgcactcatcgggttgcagctcttcatgggcaacctgaggaataaatgtgtacagtggcctcccaccaacgcttcccttgaggaacatagcatagagaagaatataactatggattacaatggcacacttgtaaatgaaaccgtgttcgagtttgactggaaatcatacattcaagactcaagtaagaaccatcaatatgtgtacttatcctttcatagtctgcctgcttcctgtctagtcacagactaaacatgatcacaaggatgttgtagtcacttagaaaattatctgatgtaaattttgaaaatatttacacaccttatttcttttattattctttaatctttctttactttaatctttcagtcattatcccacttccagtctgccctctgactagtcctcatcccatacctcctccctcctaccccatttccaagaggatgtccctacaccccgcccacaccccaccccaccaaacctccccactccctgggatctcaagtctctcaagggttaagtttatcttctctgactgaacccagacctggcagtcttctgctgtatatgtgtcaggggcctcatatcagctggtacatgctgcctggttggtggctcagtgtctgagaggtctcggggatccaggttagttgagactgctggtcttcctatggggtggtcatcctcctcagcttcttccagcctttccctaattcaactgcaggagttcctggcttctgtccattggttgggtgtatctgcatctgactctttcagctgcttgttggggctctcagattgcagccatgctaggctcctgtccataagcacaccatatcatcagtaatagtatcaggccttggagtctccccttgggctggatccctaattaggctggcactggacgtcctttccctcagtctcttctccattttagttcctgcagttcttttagacaggaacaattctgggtcagagtttttgactttgggatggcaaccccatccctccacttgatgtcctgtctttctactggaggtgggctctacaagttccttctacccacggttgggcatttcacctaaggtccctccccctgagcgtctctcaccttcctggtctctggtacattctagagggtcaccccacctcctacctcctgaggtggcctgtttccattctcttttttctgttgttcctatctactcatttcacttcttgaaaggatacaaaatcccaactaagttatagggggataatgaacgtcgctttatttctaatactgctaatacttttttttctttttttttttttttttttttttttttttttttttggtttttcgagacagggtttctctgtatagccctggctgtcctggaactcactttgtagaccaggctggcctcgaactcagaaatccgcctgcctctgcctcccgagtgctgggattaaaggtgtgcgccaccacgcccggcgctaatactttttgaatgagtagttgcaatgggataatggtttttctgaggatactgtgtgagcatacaacctactggacacaacctgaggttgtttcttccttgtcaatctttgatggaaaggaaatggatggccaaggaaagtcttgcagacatggctcctctagtaatgaaagatgtgaactcttgagaactagggagaactgataagatttgatgttcgcctactctaattgttttaactttaatatttaagatgttctacatttttcattaatttgtaagaaatattatttgttttatattcaattctaaaatatttcatttcattaaaatattcagatagttgatacaatttgtgtcaattgacatatatactacgaactgaaaaatgtaagttataattaatttgaaagttttaacagggattgaaaatatgaaaatagaacaagaaaatatatcatttttttcttttgaaatatgtagttatttttgagatacccatcattgtcatcttctttggatgatgaactttttctttattttacaaaagtagtagaaaatactgataccttttcataaatgttccctaagtacccatggttgtcactaacattccactaataatgaaaagaaaaaaacctcgtacagttgattatcgatacctttaatttaataacagaatttgaaggaagaaaaggaaaaaactcatgtgtttaagtggatgataagatacttgtaaactcacttagtgaaccaaaaagtaagacctgactttcctactcttcttttaatgtgtttcacaatgcaagtgtgtatcatgtggctttctctttttttcaaacaggatatcattatttcctggagggtgttttagatgcactgctgtgtggaaatagctctgatgcagggtaagctgatcacgcgtgtgagatgtatgtgtagtatgtccccatacatatgtttgtatatggatgtgtctgtgtgtatctttggctctagaaattgactgataacagcaactaactttatgcatactaattgtcagtaactatgatataaataaaacaagaaattatctaaatattaaagtttcatctgtaaagcaagaagcaacactttgatctgtaatgctattttcttggtgtacgtataaagctatgaatcacctttggagacttcacagtgaggggaaaaggttactaagcaatatcaataccttgcagctaataagatagatactatggagtcaaaggtgggaaatgtatcagcaattcggattccacacgcctgtgtcctttaagtagtgactagattattttgtaagtgtaggcttctaccagagtaagaagacagcagaatgtcttgtcatactttctttttccccatttgcagccaatgtccagaaggatatatgtgtgtaaaagctggtagaaaccctaattatggttacacaagctttgataccttcagttgggcatttttgtccctgtttcgactgatgactcaggacttctgggaaaatctataccaactggtgagaatctgaaaatttaagtaaaagcgtgtaagaacacaaactgagtcatgaaacacaacccgcagcatgatacagtagagaaagaatgactcggactcctctaacccattgattacaatacgcattaaatggagtaacgccaaggagagtaattctgttctgtttctccatgccttgaattctatttctcattcctcttagatacaatttctcctaagcaaaagccaagtcttccctctgccctgtcctctgctcaagtattaccttttccattggtctctttaatttccatgaaaggctcagctttttcatggtggtccccattgctttctatcaactgttattcctggtgaatccaagaatcccttattatgactatgaatgcttatcttaaagaagaaagaagagatagaggttaaagaaatactgatgggacacaatgttaattttgtcagagttctgccacatacacaagtaagttaaaatgtgaagtcacccaataccgacataattgtaaaaataattctcatattcttgtttctagacattgcgtgctgctggcaaaacctacatgatattttttgtgctggtcattttcttgggctcattctacctgataaacttgatcctggctgtggtggccatggcctatgaggagcagaatcaggccacactggaggaggctgaacagaaagaggcagaatttcagcagatgttggagcaacttaagaagcagcaagaggctgcacaggtactcagcagttcaagtaggtcttatctccgaccttcatttctctagtcttctggtgtcacctctttcactgccatgttacaaagagctgtaatgaaacaaaaggcagagtcatctgacaggatgtgagaaagctgagcacatcaaactccaaggacttctatgtatgggtcatctttttagtttgaattggcctagtcacatcactaagttgtacagagctgttttacactagctacagaagatgcttcttatgttatttttatcactttaaatagaatcaagaagaaaagagaaatagtaactttgggcttttttgcccccccccaagtttaaaatcttttactcaaatatactagcattagaaaaagtatcataagacaacaatcttttctgacaagcaaattcattagttgacatataaaacccatttggccatttcattaaagtaatttattattttctttattcagaaatacaatgtctttaggtggggttgttcaatttccttttaataaaatgttctcttggtcaaaaccatccactaggcaaagaagcatcattagcaatatggagtactacaagaggtttacttgacctttccatggcttgtcagtgtacctgactcactgaatgaaaaaaaaaaaaatgaatccaattgcccacgtttccttgtggtttctaaatgggacatacgaattcattatgtgtattttcaaaaagactaattaaactatttaaatcaaatttatttgtaaccatcttgaccataaaaaaaaaaaagagttacttcaagcatgctaatttatctgctacagttctagattaagggaattgtgggaagaatgttgactaagaagttctaacttcttcataacagaacaagctgtgaccacacagcatcttaccccataactagtaagagccaacattgatatgaagatagttgggatggcttttagagaactgaacatcttttaacttgcttagcagaaagctctcaactctctcctttctatagataactgtatcaggggagagtgaggaagaaactgtgatatgtttcataacgctgattatgtggaacatctccacatatgttgatactatgtggaagatattgaacactttgataagcatggaaacctgagaggacccctgctcactacctcattaccagctttgaatagaatcttgagttattcttgactgccaatgaatcttacgaatctctgcggcatcagacaaagttaataaatgttaccatggagaaaagtagattatttttaaaattcattattagttaaaaagaaaaaaaaacttcccttcatacctggaaaacatcatctagatgttggtttcagttagcttgcattttggacatgacacaccaaacttgagcatcttaaagatttaatgcagtcgtttcttgcaacacactgaattggtttctcctctatatagcaggcagcggctacaacagcctcagaacattccagggagcccagtgcagcaggcaggctctcagatagctcttcagaagcctctaagttgagttcgaagagtgctaaagaaagacgaaatcggaggaaaaaaaggaaacagaaagagcagtctggaggagaagagaaagatgatgatgaattccacaagtctgagtctgaagacagcatcaggaggaaggggtttcgcttctccatagaagggaatagactgacatatgaaaagaggtactcttccccgcatcaggtacgcactgcagagttcttggatacaccgatgagagtgagccatctgagagagaatttaaatagatggatgggttttcatcaaccgaattgactactgttgcattacacttaaaactgttttttcccctctatgtagttatgtgaaagttttacttcccatccacatatatgcaaatccactcttaggtgcttttgaaataacacaggattggaagacaggagaactggcttttcatgttaaatgtgccttctggtaattgtaatgacatttaatcttgcagctattctaaccacatcaacaaaatgtagactaggggttagctactcagaaatgtacctttggttaatttaaggcaccatgttggttgtcatcccataacttctttatcaattatcccatgaactttaaaaatattagccatgataaactttatagaaaagtgcatcattcagaatttcgaaatttttactttctggttctacaagtaatctgttttacagctgtgtcaaaaaaataaataacatggttttaggaaactgaactaaaatagtctgtgaaaactattgttggaattatccaaaaggagagagaaattaaaaaccgttgctgtcttgctgcccatgttcattgtaggtggatacatacatagagagatttttaaggatgtctctgtttaaatttgagacgtggtggctgggtatttttttttctcccattacttttaggctgcactccagctatctgttatttaatatcaatggctggaagccatactgtatagaactaatttaatctcatctgaatgccaataaattatattaatcagaaggaatgtgctaccctctctaaaataacaatgcaatattcttcaacttaaacttgcaaaactatttcaacaggcagttctaatgtaattttactttttgtcatgtgtttcaaactttcagtctctgttaagcattcgtggttccctgttctccccaagacgcaatagcagaacaagtcttttcagctttagagggcgagccaaggatgtggggtctgagaatgactttgctgatgatgaacacagcacctttgaggataatgagagccgtagagactcactgttcgttccccgaagacacggagagcgacgcaacagtaacctgagccagaccagcaggtcctcccgaatgctggcggtgtttccagccaatgggaagatgcacagcacggtggattgcaatggtgtggtttccttggttggtggaccctcagttcccacatcgccagttggacagcttctgccagaggtgataatagataagccagctactgatgacaatgtaagaaagttttacatagttcaggcatggctggcctatgattgctgcaccagccagtgtttctacagaatggaactcttgagaatgattcctggttggtcgagctgagaatgcacctgcatcttgtagcacctttaatagactaaccagctaaatgttagcgcctcggcactccttgcacaaacctgaatgcattacctactgtatatgctaaggattaattgaacaataattacttccttggatgatttgctatgtacccataccgaatttacatatgaagacatcacccctagtgccctattattgtaagattaaatgaatgactttttagtgtaaagggtgtatagtaaatcttcccactaaactcttacagtttatctgggctttagttcagattttcaaaaacactgtgcttagatagcttcatcattacctacattttctaaggatcatatttatctcactaagaaagaaatgctgatataggtattcacaaaacaatctacacagagacaccaataaattattaatccagataaaataattggaaatatatcagcatgacgaggcatgactcggtgtaaatcagatgacttattgctttggtatcctggatgaaactgttgcccctgcatcacatgcatggatcagcatatactttataataaatgagtatatgtcttatgcttagaaattgagatgacagctaagtgtgttatggtcttcggtattttcttaaaattaaacagctactttctcgaaagtgaaaaatggcctgaccatatataagattttaaggcattaagatttgacagtatgttcattaattgaaaacaagctataaatgcctatgcaaagctttactaactatattttgatcgcatcctatttcataagagtataaaatcaaggaggggtcaaaattttatattttcctttatgactacatggttaactgtttgaatattccaggttaatgatccaataagtcaccagtatctgccattggccatcaaagatgaagacgcatgacgcatgtctttcatgaaaatcactgtctccatttatttctgtgctgtattgctcaagctagtcatgtaatgccttagcatttgctttgcagggaacaaccactgaaactgagatgagaaagaggaggtcgagctctttccatgtttccatggactttctagaagatccttcccagaggcaaagggcaatgagcatagccagcatcttaacaaatacagtagaaggttggtaacaaaccctacctccctcagttcagccatcttgcaatatcttgttggagtcaactctctctacttatgtgttggcaagtacactctatgaacaaaagtaaaaggaacagtcctactagcaaatggctggtgatgtattctgaaagtattacatactaaattcaatgttctaaacttctgaacttttaatgtcttaataagtatgtgggaacagctagaggtttctaatccattagtcacaaatcctttgttgtaatgtgcttcaaacttcagcagcatggccccttacattatgatctgaaataattttaagactggagaaaagaataagtcactttcacaaaatcaaagcataaagaatgatttctttatatattacattaatccttgtcctaaatcttaaaattgtgtagcctatcagtaacaagaaattttacacagagtgaatagttccaggctatgtttccatggaacctttgtctatatgcccaaccactgtaatagaggccaagtgttaagcaacactgtctatgcaaaaacaaaacaaaaagttttctttcctatctctatgtacatgactaatgatactgtaccattttatcaagtaattatctttgcagcaaagagaaatatgtttgctactgagagcatgtttagtggatctaaaataaatttgtaaatttgtaagtcagtaatggtgttctttcagaatctaaggccatgatcttctttaaatgatagaatcactgtgtcctagctctagatggttcaaataattggttattgtttcttctattttttttttaaatctagaactagaagaatccaggcagaaatgtccaccctgttggtataaattttccaacatattcttaatttgggactgttctccatattggctgaaagttaaacatattgtcaacctggtggtgatggacccatttgttgatctggccattaccatctgcattgtgttaaatacgctcttcatggctatggagcactaccccatgactgaacatttcaaccatgttcttacagtgggaaacttggtaagtgtgcaagaaggtaaataaatgagcttggtcttcaaattttctgcttaagaaactgtttattatcttcaatagtttattagacctctttcaaccacaattcctgcatccctaaggcaataatatccagacgggtacacactaccttttgccaaataatggcctttaatgtctgtgcaaaaactgagaaatagaacatagacctgtaataataatttaaaaagtcagtaatggtgttctttcagaatctaaggccatgttcttctttaaatgataagatcactctgtcctagctccagatggttcaaataaggtcatgagttctcttccttatatgctctttcaaagaatgataacacaaaagtgtctccatcttttttccttccagggtcaagggagtctaagttctgttctagattcatatcatgaaatattgacagtacctagtgcattttgtacatcatatgattagtaagcccatttttagtgaaagtttcgacaatattctccattttttactttgaagcataaattgttaaaatattttccagcagactcaacttaaaagtctcaggtttttatttgaagaaagaactatctaaagtataacatgcattctatataatatctaggagaatgtttagtatcttgctgataaagacaatgtaaatatgcatatgctatagtggatttatatttttaaatctctactatttagattctggaaacgcacttttagtgcatgtgactaatattaatgatagtgatgagaataatataagatacacatgatttgaagggaataaagataaacttacaaataaaatgttttggggcaaaatcttgtctaggtgacatgaggtctggaagttggttaataatagatgggcattttatataccttgaaatattgttctcttcatctgtaataaaatagttacagcatttacactatctggttattggagcttctatgagcataaaacagtaaactaatataaaccagcattgtcaaataaacttgtaggataatcttaggatcaataatggaaaagtagaagtagcattagtcttttatatatatatatatatatatatatatatatatatatatatatatatatatatgtatatacatatatatatataatattatctttgatgttatcactatctaagataatttaaataatatggttttgcttttaggtcttcactgggattttcacagcagaaatgttcctgaaaatcatcgcaatggatccttactattacttccaagaaggctggaatatctttgatggtttcattgtgacactcagcctggtagaacttggccttgccaatgtggaaggattgtcagttctccgttcatttcgactggtaaacaagacaactgcaataataactgttttccccagtggtgttctctcatagtgcttccctatacttttccacaaaactattatcattttaagtttttgtgtaggctgatacctctaaatggggaataatatctctcaggtaatatttgtatatttttttacaaaggcaactttcttctgaaaagcctatttatgtaaactttctaatattatcatttataagtttgcagtgctgggttacataacccagagccttgagctgtcactagccccgtcacgtgaacactgatatctttttctattgctgtgttgataatgtgttctttctaatgacttggacaattaatactttcaacctgaagaagtgtgtttaatagaaatggttaataacttaataaatgttgaaaatgtaaataactcttcgatattcacataaaatgtatgcatgtaattctacttctaaaaaatatgttccataaaaacaaaagtttgcaggtctattaaggatagatagatagatagatagatagatagatagatagatagatagattcatacaaattccctgaagattgaggaagagtcatagcaaataatccaggtttaacaatacattgcatttatttatatttttcctctgttataatattaatagataataaatatttttcattttattttcatcaatgctctcaatgcaatgttacttagaacctttttgacaaatgaatcctttggcagaaattattcacattcagatatcaatatgacactataaaacttggaagatggtctcagagaaacaaattaaagaatatagtttctaatatgcaactaggaaatcactttcagagaagatgagccacattattaaaatctggaaaagctagattcttcctatgagggtctaaatattaactatgaacctgagaaagttagagcaagataatgtaagcagaattttttaggggtgtgtagtgctagcaacggatctcatggctgcatgcccactaaggaggtgttctgtctctgagctgcagccccttattctttagtaagtctcaagataattaatgcttgtgaagatcataataatgtatattaatttgttctcattttctagctccgagtgttcaagttggcaaagtcttggcccacactgaatatgctcattaagatcattggtaactcggtgggagcactgggcaacctgactctggtgttggccatcattgtctttatttttgccgtggttggcatgcagctgtttggaaaaagttacaaagattgtgtctgcaaaattgccactgactgcaaactcccacgttggcacatgaacgacttcttccactcgttcctgatcgtgttccgcgtgctgtgtggggagtggatagagaccatgtgggactgcatggaggtggcaggacaagctatgtgccttactgtcttcatgatggtcatggtgattgggaaccttgtggtatgtacccaagttagatatgcatttcagaaatacatcaataacataacaaatttgtgcccaatttattaaaaaatgactttcatgataaataattttaaatgcctctgatcttaagatatgtttatcttcttattctaaaaatataccgcaacatggcaaagtatagtatcacaatctactatggtaatatagaaatacatcttaaaacaaatgtatgcacattaatattacctatgatattttactctcaaatccatattttacagctctatttttttaagacaaggtttctttctgtatccctggctgtcctggaacttgctctgtagatctagctgggcttgaactcagagatccacctgcctctccatcccaagtacttggattaaagtgattgtgccacctggcttctcaactctattttattcagtcacttcattgcatttagttattaaattgcataggactgttaaaaatttacacaaatatgttatgtgcgtgtgtttgtgtgtgatgtctatgtgtgcgtgtgcatgtgagggagaaagggggcaatttttgctatagaatagtgctaacaagaatggtccatgcatatgttgaagactttcattctaaatttggtcatgactatgatttttttcagttatcatccaacaacacaaaatcatgaagagagaaaacccaaaatacatttaaataaataattgcaaatattaatcatttttaaatttggaattgttaaagtatctcagtaaatatatcttcttttactcatataatattaaatataatgataaactaattcattcaagttcttcatttcatgtatgggagatcttgagagcagtagaaagaatgcctcatgtacatagagatggagcaatatcacaaattcagagtgctcaaaacttttctagggcggagtgttgaacccagggcctcatgttttctggtcttggcccacttaacttctagcaatgcctgtgctgtatcttgctcatatagcattcggttattcattctatagctaaaggaataagccatcctatgtcctctgtgttgtggtgaacacatacttatgtctgttttcaaggtcttgaacctctttctggccttgcttctgagctcatttagtgcagacaaccttgcagccactgatgatgacaatgagatgaacaacctgcagattgctgtggacaggatgcacaaaggaatagcttatgtaaaaagaaaaatatatgaattcattcaacaatcctttgttaagaaacagaagattctagatgaaattaagccacttgatgatctaaacaacagaaaagacaattgtatctctaaccacacaacagaaattgggaaagatctggactgtctgaaagatgtgaatggaaccacaagtggcatagggacgggcagcagtgtggagaagtacatcattgatgagagtgattatatgtcattcataaacaaccccagcctcactgtgactgtgcccattgctgtgggagagtctgactttgagaacttaaacacagaagactttagcagtgaatcagatctagaagaaagcaaagaggtaagatgctgtaaattggggagatgtatatgtataaatatatgtatggattatactttctgtattaatgattctatagaacctttttgatataatatcaaatattattttattgagattccaaaaatcttttctcagggagatgaatgccaaagcaaaatgtaatatactgttgtatgagttttctcattcttaaaatctgtttgttaaatgggtacaaataatgaattaaaaactagcatcattagttacttaatccttgtaatgaacagaaaggaacatattactgtaaagtttcaagtgaaaaaacaaaacaaaacaaaacaaaagaaaaaaacaaccagctttggctggctaagagcatgtaacctgtgaggggtggaaccccactttgaacctgaagcctttgtctttaagttttaatatttttgtgtgtgtcatctcagtttaaataactgagtgaacttcagaaaaaaagtgctccctaccaattgtcacattaaaattacataatgtcataaaatagcatggtgctaagaaatagatggcttacacacacacaaaaaaaaaaatttattcacaatctggaaagcagacatttcaagaccatggtactgcagggtcagtgtctggggaggactatttgctaaggttgtagatgtccaccttctcactatgtctttacaaggcctgactgataggtgcactgaacagaaatggatctcttgtctcttcctcaacttgtaagggaattactctattggatgagaatcttggagtcattagataagtagagctcattatcaagccagaccttcagatatttagagactctcagtttcagcaattgaatttaggatagaagaatgggtaggggaatgtaattcagtccatgaggtcctacttgaaaatattaaagctaaaacatttgtggatattaggactgtctctaacttgggctaacttaccgttactatttctgaacaaccaacacattcctgtgtatattgctagctatggtgttcagtggtttgtgtgtcctgaatggtttttttttctctaactacattttcatcttacagtgggatttctgtgatataatgctattgcatggaagtctcaaagataacaaagcatatttagagcttgagtaatctaaatactttaaagacactctattcaaagatactctattcctattaaaaatcgactggtatatcaaccataagcaagggaaacaatggtgtgttttggttggtagttcctagagcagaaagcatagacacatgacatgatattaggttgttgaatatgtctatttaatgatgattcaagcttttataaaaactttggtgtcatattggaattcccacttttagaagtaggaactgatattaaaactagcagtggctgcattgagatattttggggtcttctcgtgaaagaagagaaagcaaaataacacagaagtgagacagtggggaaactactaactccaatgagatggatttagtgtgggaggaagatggacttttttttttaacttactgaatgtcttaaggactactatgtgaacattgtcttatttaaaatgtttacttagtacctggtgttttctaggaaataaatggtagatgtcaactgtgaaataaatgtttgttgagcatggtacctaatgactaaaagcccatcattgggcaggtaaagtcagaaagatcaggaactcaacgtctcagctagatagtgagcttcagggaagcctgaaacatataagatcacatctaaaagtaaaaatatcaacaaaacatcttatttaaacacatcattctaaatttctgacaagataaatttccaaaattcgttctagtttgaatctcctcatcttaagtccttgaatctataggcgtacatttaatattggctgaataatagtagcagctacttattatgtactgaatattactgataagcaattgtattgtaaaaagtatccataaaattatctttccactgaaaattaacatgattcgcatttgtacagcttctgtttgcattaatcaggaatagaaagagcactgacatcttcatcactgattgtccacagaacccctagagcccatagccattcttttcccatgctcttgtagctcagaaatctgctgatttaaaaatcatggatattacaatagtataaacattagagatgtaatgtttctaaattacaagccatcgcccagttagagtttaaaagccaatatgttaccccattttttttaaattttagtatattagaatagattgtgtcaatgcaatttgcctattgtgctagtacccaaacagggtgtatggagaataccacctgtctttatattgagaagttagggtagtagagaccgacattagggaaactgatgatatggggcactaagggctggtgtttgagcttccctgctgctttataagaactactgtctcaatagtgtctttttctttgatatgacagtgttgctttgttgagagtcattaatagtgatcattttgattttaatgcatacattagaaatatctcctaaggcagaatatacacaacaacatattaacattcatatatttaatgctatggagtatgatggctaatctgcattgtctacttggctaaatgtagaaccacctagaaaactgtaaggtgcaccccaaagtacatctattagggagtttatataaagggttaactaagaagaaaactatgtttaaatggaaatgacacccacccagggactgggagcacagactgaataataaaaggagagggggaagaagaaatgaagaaggagaagaagaagaagaagaagaagaagaagaagaagaagaagaagaagaagaagaagaagaagaagaagaagaagaagaagaagaagaaggaagaggaggaggaggagcaggagtaggaggaggagaaggaggtggaggaggaggaggaaaaggaagaggaggaagaggaggaagaggaggagggggaggaggaagagggggagaaagaagaggaagaggaggaagaagacgaagatgacgacaatgatgactaccagcagaggctgctttccctttcttggttttcttgctcaccatgacatgcactgctctaaaccaacatgctctctccccaccatcatgcactgcctgctcagaaactatgagccaaaagaaaacgttcttccatttaaactgctttgccaagtattttaaccctttgagacaataataactgataaacagagcatcaggaatctcatttatatatttttgtataaaagtatcaagtcagcgaaaggctaaggttaaaaacacgtcatgctttgttcatagatgagtatgggcttcatttatagataagttccttccgtgctagtctttgttgcatctctctctctgtacatatgacagtgcactatatgacccatcagttacaaaatcccaattatttttatcacttattaaaaagtaaactttcgggctcagggggtaagagcactgactgctcttccaaaggtcctgagttcaaaccccagcaaccacatggtggctcacagccatccgtaatgagatctgacgccctcttctggtgtgtctgaagacagctacagtgtacttagatataataataaatcttaaaaataaaaagtaaactttcattaatatacattaataatatccctgtaataaactaggtcacaaaataaagacatctcacgtttaaagttaaagctgagaagaaagagacacctgactggttccttccaaagaaaccccaagctgtggcttgttctgttgctgttttgttttgtttcattttcaaggtcattttctctaccagagaacatctttaaaagcaaaaaaaaaaaaaaaaggaaaaaaaagggagcatgaatccacggtgaagggaaaatgctcttaccatcagggtcgcagttagtaactgatcatctcacctcataaataacctgccaagtacatatggattgcacagatcagacggtttatatggaaaaatatacccagcctctaattaactagtccattagggaattaataaattgaagattgattaatagcatattaaatgctaaaatggatttaacatattttattaatttcaaaacactaagaaacatactttaaaaagcaaaaattaataccaacttaagaataaaaaggaactagtctacttcattaagttttaaaataataaaatacttgtttgacaacactgggttgcattgttacctaggatctttagaatattgtatgaaacatgtctgtgtgcaaagttgaggtgagcccctccctcagagctattgagcttcctccctatttgagtagcagtgataacttaaccaaagaactgcacttactttttgttttaacaatgagataaataataataagcatacctttgcttttagagttctgaagcaatgttttatctcatgattaaaatgtaacaaattaaatgacgacattgagactcctttattttatttcatataataagacttgcaaacacacacaaacacacatgcacacacatgagtatttgcttttatgtctatctgtgtactacttacatatcttcttcccacaagatcagatgagggcgtcagaacctctggaactggagttttagatggttgagagctgccatgtggatgcggggaattgaactcaggacatctggaagagaagtcagagctcttaaccgctgaaccatctgtagcccattactagtatttttaatgttagtattatccacaagtatccatagatgtttacaaaaaggaacagtatatcggaactcaaagtaaagcttagaaaatcaagatagcagatattgtatatggctatctgacctcaggtcagttaaactgaaggtaaaatataagtcagaagacagaattgcatttaaaaataaggagattaggcctttatgtaaccaaaattccagtaaaggtggttgagtttgtacacaccagtgtttgttgtctttttatttttttgtcttactgtcccattatatcctcactagaaagaaatatttcgacaactcaaaagaataaagtttgaatagaaataatgtaagcctcagactatctttatgaattaagatgccaatataaaaataacaaggaaaaactgtcagtctgcatatattctttacaaagactttggaagagtgaaaattttatgtaggtagcctaaaaaagtcaacatgtgaaatatgaatgtaaatacagtctaaatgaattttttgaagtgcatatctatttgccttctacataaaatgatattttgaaaatgtgtcaaaagataacatagtgccttttgggttcttgcaattttcattccaggcatttctcagataagcctttttctttttttccattttttattaggtatttagctcatttacatttccaatgctataccaaaagtccccatacccccccactcccctacccacccactccccctttttggccctggcattcccctgtactggggcatataaagtttgcaagtccaatgggcctctctttgcagtgatggccgactaggccatcttttgatacatatgcagctagagacaagagctccggggtactgcttagttcatattgttgttccacctatagggttgcaattccctttagttccttgggtgctttctctagttcctccattgggggccctgtggtccattcaatagctgactgtgagcatccacttctgtgtttgctaggccatggcatagtctcacaagagacagctatatctgggtcctttcagcaaaatcttgctagtgtatgcaatggtgtcagcatttggaagctgatcatgggatggatctctggatatggcaatcactagatggtccatcctttcatcacacttccaaactttgtctctgtaactccttccatgggtgttttgtttcctattctaagaaggcaaagtgtccacactttggtcttcattctcttgagtttaatgtttttagcaaattatatcttatatcttgggtatcctaagtttctgggctaatatccacttatcagtgagtacatattgtgagagttcctttgtgattgggttacctcactcaagatgattccctccaggtccatctatttggctaggaatttcataaattcattctttttaatagctgagtagtactccattgtgtaaatgtaccacattttttgtatccattcctctgttgaggggcatctgggttctttccagcttctggctattataaataaggctgctatgaacatagtggagcatgtgtccttcttatcggttggggcatcttctggatatatgcccagaagaggtattgctggatcttccggtattactatgtccaattttctgaggaaccgccagactgatttccagagtggttgtacaagcctgcaatcccaccaacaatggaggagtgttcctctttctccacatccttgccagcatctgctgtcacctgaatttttcatcttagccattctgactggtgtgaggtggaatctcagggttgttttgatttgtatttccctgatgattaaagatgttgaacattttttcaggtgcttctctgccattcggtattcctcgggtgagaaatctttgttcagttctgagccccattttttaatggggttatttgattttatggagtctaccttcttgagttctttatatatattggatattagaacccctgtccgatttgggataggtaaagatcctttcccaatctgttggtggtctttttgtcttattgacggtgtcttttgccttgcagaagctttgcaattttatgaggtcccatttatcgattctcgatcttacagcacaagccattgctgttctattcaggattttttcccctgtacccatatcttcgaggcttttccctactttctcctctataagtttcagtgtctctggttttatgtggagatccttaatccacttagatttgaccttagtacaaggagatagtactggatcaattcgaaatcttctacatgataacagccagttgtgccagcaccatttgttgaaaatactgtcttttttccactggatggttttagctcccttgtcaaagatcaagtgaccataggtgtgtgggttcatctctgggtcttcaattctgttccattggtctacttgtctgtctctataccagtaccatgcagttttgatcacaattgcgctgtaataaagctttaggtcaggcatggtgattccaccagaggttcttttatccttgagaagagtttttgctatcctaggttttttgttattccagatggatctgccgattgccctttttaattcgttgaagaattgagttggaattttgatggggattgcattgaatctgtagattgcttttggcaagatagccatttttactatattgatcctgccgatgcatgagcatgggagatctttccatcttctgagatcttctttaatttctttcttcagagacttgaagttcttgtcatagagatctttcacttccttagttagagtcacgcctaggtattttatattatttgtggctattgagaagggtgttgtttccctaatttctttctcagccagtttgtcctttgtgtagagaaaggccattgacttgtttgagttaattttatatccagctacttcactgaagctgtttatcaggcttaggagttctctggtggaatttttagggtcacttataaatactatcatatcatctgcaaaaagtgatattttgacttcttcctttccaatttgtatccccttgatctccttttgttgtctaattgctctggctaggacttcaagtacaatgttgaataggtagggagagagtagacagccttgtctagtccctgattttagtgggattgctgtagattgcttttatcatgtttaggtatgggccttgaattcctgatctttccaagacttttatcatgaatgggtgttggattttgtcaaatgctttctcggcatctaacgagatgatcatgtggtttttgtctttgagtttgtttatatactggattacgttgatggatttctgtatattgaaccatccctccatccctgggatgaaacctacttggtcaggatggatgattgttttgatgtgttcttggattcgattagcaagaactttattgaggatttttgcatcaatattcataagggatattggtctgaagttctctatctttgtgggatctttttgtggtttaggtatcagagtaattgcggcttcatagaatgagttgggtagagtaccttctgtttctattttgtggaatagtttgtgaagaactgggattagatcttctttgaaggtctgatagaactctgcactaaacccatctggtcctgggctttttttggttgggagactattaatgactgcttctatttctttgggggatatcgggctgtttagatcattaacctgatcttgattcaactttggtacctggtatctgtctagaaacttctccatttcatccaggttctccagttttgttgagtatagccttttgtagaaggatctgatggtgttttggatttcttcaggatctgttgttatgtctcccttttcatttctgattttgttaattagaatgctttccctgtaccctctagtgagtctggctaagggtttgtctatcttgttgagtttctcaaagaaccagctcctcgattggttgattctctgaatagttcttcttgtttccacttggttgatttcgcccctgagtttgattatttcctgccttctactcctcttgggtgaatgtgcttccttttgttctagagcttttaggtgtgttgtcaagctgctaatgtatgctctctctagtttctttttggaggcactcagagctatgagttttcctcttaggaatgctttcattgtgtcccataagtttgggtatgaagtggcttcattttcattgaactctaagaagtccctaatttctttctttattccttccttgaccaaggtatcattgagaagtgtgttgttcagtttccacgtgaatgttggctttctattatttattttgttattgaagatcagccttagtgcatggtgatctgataggatacatgggacaatttcaatatgtttgcatctgttgaggcctgttttgtgacctattatgtggtcaattttggagaaggtaccatgaggtactgagaagaaggtatatccttttgttttaggataaaatgttctgtagatatctgtcagatccatttgtttcatcacttctgttagtttcacttgtgtccctgtttagtttctgtttccatgatctgtccattggtgaaagtggtgtgttgaagtctcccactattattgtgtgaggtgcaatgtgtgctttgagctttactaaagtttctttaatgaatgtggctgcccttgtatttggagcatagatattcagaattgagatttcctcttggaggattttacctttgatgagaatgaagtgcccctccttgtcttttttgttgactttgggttagaagtcaatcttatcagatattaggatggctactccagcttgtttcttcataccatttgcttggaaaattgttttccagcctttcattctgaggtagtgtctatctttttctctgagatgagtttcctgtaagcagcaaaatgttgggtcttgtttgtgtagccagttttttagtctgtgtctttttattggagagttgagaccattgatattaagagatattaaggaaaagtaattgttgcttcctgttatttttgttgttaaagttggcattctgttcttgtggctgtcttcttttaggtttgttgagggattatcgtcttgttttttctagggcattgttcccgttcttgtattggttttttttttttttcctgttattatcctttgaagggctggattcgtggagagataatgtgtgaatttggttttgtcgtggaatactttggtttctccatctatggtaattgagagtttggctgggtatagtagcctgggctggaatttgtgttttcttagtgtctgtataacatctgtccaggctcttctggctttcatagtctctggtgaaaaatctggtgtaattctgataggcttgcctttatatatgttacttgacttttttcccttactgcttttagtattctatctttatttagtgaatttgatgttctgattattatgtgtcaggaggaatttcttctctggtccagtctgtttggagttctgtaggcttcttgtatgttcatgggcatctctttctttaggtttgggaagttttcttcaataattttgttgaagatgtttgctggtcctttgagttgaaaatcttcattctcatccactcctattatccgtaggtttggtcttctcattgtgtcctggatttcctgggtattttgagttaggatctttttgcatttaccattttctttgattgttgtgccgatgttctctatggaatcttctgcacctgagattctctcttccatctcttgtattctgttgctgatgctcaaatctatggttccagatttctttcctagggtttctatctccagtgttgcctcactttgagtttctttattgtgtctacttccctttttaggtctagtatggttttgttcatttccatcacctgtttgtatgttttttcctctttttctgtaaagacttctacctgtttgattgtgttttcctgtttttctttaaggacttgtaactctttagcagtgttctcctgtatttctttaagtgagttattaaagtccttcttgatgtcctctaccatcatcatgagatatgcttttaaatctgggtctaggttttcgggtgtgttggggtgccctggactgggcgaagtgggagtgctgggttctgatgatggtgagtggtcttgggtcctgttagtaagattcctacgtttacctttcgccatctggtaatctctggagttagtagttatagttgactctgtttagagattgttcttctggtgattctgttactgtctatgagcagacctgggagacagattctctcctctgagttttagtgctcagagcactctctgctggcaagctctcttacagggaaggtgcgcagatatcttgtgtttggacctcctcctggccgaagaagaaggcccaaaacaggacctttctcagaagctgtgttgctttggcaggtcccagaagctgtcagcttctgtggtgcagactctcacctgtgcagactaatttcctaagttctgcagagtcctggaaccaagatggcgaccactgctcctgaggctgaggccgccttccgagccaggcggacacctgtcctctggtccggacggtggccggctgtctgcggcctgcaaagggtgctgcctcagtggctctgtgcctccacctgtcccagaagctgtccggttttgtggcgcaccctctcacctgttcagactaatttcctaggttctgcggagtcccggaaccaagatggcgacagctgctccttaggctgaggctgccttctgagccaggcggacacctgtcctcttgtccagacggtggccggctgtctgctgcccgcaaaggatgctgcctcagtggctctgtgcctccgcctgtcccagaagctatccggttctgtggcgcaccctctcacctgttcagactcctttttcttagaacaagaaaaatagtatttagttccatcaactaagtaatcttttaaacgtagaataagaagtcagtacttcacacgtcaatgaaaacagtggtcattgtgaaaaacatgatatggctggcaagacgtagaaccagctatatgcagtatcttactagcaaatgccattgttgtagaaaggctgcttgagagcgttttgttctacttgacactgaaaaatgatatcactgaagattaggttttatctgaatggtacctgatattgtgattcttgacagagagcaaaatatctgtagagatctaacagatcactgttacatgctcagcaagttaaaatttctatttacaataaagtttatctgaatccctgcccacacataagtttattcagagggaggaagatgggatttctagtaattaatgaattttggtaaggactcttaacttctcaaatcaatttacttattatacaaagagaatacacacagacagacatatatatagagagagagaaggaggaggaggaggaggaggaggagaaggagaagaagaagaagaagaagaagaagaaggaggaggaggaggaggaggaggaggaggaggagaagaagaagaatgctggataatgataaaattctactcacagggttttttttaggaaataagaaataattcttgtagcacattacagccaattatagattccagttatgttgaggaaagacacaggaatcctcagaatgggcactgtaactcaagcacacagactagccatttattatagtctcttcacttattatatggccaaatgcatgacttatccttggatatgtttttaagtcagaagaaatggttgaatcattcaaagcaaatgcagtgtagatttaaagtctacctgttgctgtatcctgtgcagaggagcaaattctccagatgatgaatatgtaaacgtgctatagtcataattatttaaaaacttaaaactagcttccagtgaagaatctatgtataataacagaggtaatctttaaaacagacactcagaaccatgactggtctttcatgtcaatgcagatccttgtgacctttaattttctatttaacaagaaatttaaaaggaagagagacagtcagctggatagaggcccaggtgtagaagccatcttcctttgatggaacaaagaaaactgtttcatgtatacgaattagtagtagtagttttagcatgatgcttaaagtttattatttaaaaatatgtgttgactgggtagttggtgagatctcttttagattaaacataaatccatcgtttgaaaaacatcgtattttatttcaagtatctaatagggctacggattttatagttgccaatgaacaccaagtttctagcacattgttggcgctcagtgtatcttaacttgactaaattgccaagtgtactatttactcttacaacaatttgaaggtcagcaggataagttgatattttctgctgtttttgccatctttgactcctaaggatccaaagagcaccatgtgtctggtgactactgccaaagtctgagaaacgctgtttctgttgaattagctttacgtgggatattagtgtgttacttgttgagcggaaactgacttttccatgcatttattggtctgtttctgcaacactttaggttcgaaaatattttccaataatgaacccctttaattaagagtctaaggctatttgtttattgatgtatttttaacttattgtaaaacaccttttctccaaaaggcatatgaaatataaaaatatgaatacatttataataaataattctgatcctctgttctaacaggaagtgtaagttaaagcattatgttataatttctcagaattgagacactattagagaaacatatgtaaatgtttaaacacagactagcttaatggttttattttcttctcaggtagagattctccgagcttcatttcccttatctctctccccattcctacaagtcacgacactctctccttccccaatccatgggataggagttccctcactacatgttgctgatggtcaatttgtgacatcccagcacagcgcagcaatctgcacacagaaattcctggggtaggaatcaacagttaggttttcccattccctcttcttctgagtcttcgttttactctcaatttacattagcaccttaccttgagaaaaataatcccttcttggtatgtcattcattggggaattaaattaaaagaatttgctttttatgatcattttatgcccagaaactcaacgaaagcagtagctcctcagagggaagcacagtagacattggggcgcctgcagaggaacagcctgtcattgaaccagaagaaacccttgagcccgaagcttgcttcactgaaggtaaagatgcgggtgacatcgcttgcagttcatacggagtacggccttttcaactgtcgatcacccaaagtgggtggcttaaatgtaatagaccttgtagagagtgaggcccacaggctagattcagtatataaaagcaatgtgaggacgtaaaagacacatcaaagcaagtccaggaaagtcacctgttagcaaactcagacaacttaggaaaccaggaacaccctttcaggagcaccacaagaatacacacttaggagaggaggtttctgtccaggtattaatttcattttgaaacccttttctagagtagctccaaccaaatgcattaatttcagaccataaaaagtcagatgcagatactagcacccaaccagtgcacagaagccagggactcccgtggttgaattagagaaaagcaggaagaagctgaggaggaggttggccccatggaagaccagcactctcaactgacctggactcccaagatgcctcagacactgagccaccaacaggcagtatacaccagctgatgagacccccaacacatatacagcagaggacttcctggtctagcttcagtgagagaaaatgcacctaacccttgagagatttgaggtcccagggagtggggaggtctggtggggtgtgggtgggcacatcctcttacatactagggtagggaaatatgggatgaggagcagtaaggggccagaccaggagtgggataaatactggactataaaaaaggattaaagaaatttttaaaattaaaataaaaaaaaagagagagatgagaaaagaacaaccacaaaaggcaaactacatgtcgttatttgtaatttacaatgagttttctttgaaggcaacattgtgtcagagaaaaggcaaaatatgaagatgctgtatgttttcattgatttgccattgcttataaattacacactttttaaaagttaaagcaaattaggaatagcgctggtggtggagggatgaagctgggctgatgcaatgtataatggaggtggtagagctatctttcatactcctattggaaccgtatgcttgcaggctgtgtccagagattcaagtgctgtcaaatcagcgtggaagaaggaagagggaaacagtggtggaacctacggaggacgtgcttccgaatagttgaacacaactggtttgagaccttcattgtgttcatgattctcctgagtagtggtgccctggtgagtgaaccttaacccaaagtgcttcagtagcaactgggaaaatctcacactctttaagcctttgtttcccattgccttagttatcaatgagtcttcaataacaacacccttttatcttcctgccagatagttaatgactagctgcatttctgggtttgcatctatgtgtgcacacacatttctaatcattcatcgatgtttgcttatcatgaaacagatcaaaaaaaacgggtcccttttgcttagtcattataaacaaagtgaatattcctctttgcttaactaaacatgactatgaggagcacatcagagttcatgttagtgctattatagtggttatatatcctctgagttctgatgaactctttgtggaagcttggtaggtcgttggggacctcttctctgtcccttttcatgtgtgttcactattcttgggctactcaggaaacaaatgatattggtaatgctaagtagtgggcaccatacctaagtgattacttaaagactggattatgtttatagaaacatgttaatttcatcagaatctatttaaatttagatgaaaattgtttacatatatacaactatattgacagaattttagatataaaaatatattgaagtcataaaataaaatgtttggagagattgataatagagaaactatagatagatagatagatagatagatagatagatagatagatagatagatagatgatatatagatttagataaatgatagaatatatacataaataaataagcacaagtgtttgcctgattaaaacacaatgacttacaagttttaaaatcaagtatatatttttctataaaattgttgatggaataactgtcttctacatatcattatctacatatctgcatatcatagacaaaaatgacttttcatcattgaaagaaatatatattagaagtaaattaccagctgaggtttatgttaggaaaaggcataggtggatctaattgaaaagtaatatgacattcctattaagtaatgtcatactatacgtaccaaaaagtaaaattatcagctagatgtggtggtgcacacccttaattccatcaggaagaggtagacagatgtttgtgagttagagccccagcctactctacaaagtgaggatagccagggctattatacagagtaaccctgtcttgtaaaacaaacaacaaacaaacaaacaaacaaaacaaacaaacaaaagaaaagaaagaaaaagtaaagaagtagaattatcaacaaaaccccaaaaaagcagttaactgcttagtaaaataaaattggacagtatattgataaatcacttattgatttaatctctctcctataaaaaaaacataataatgagagaaatttggtttttgctggaagtcaagctactggtctcacgtagacaactgttctagccactgagtcacacccacagcatagaaacaaagaaacagggtacagagctaaagaaagaattgtcaactgaggaatatagaatggctgagaagcacctataaaagtgttcaacatccttaatcataagagaaatgcaaatcaaaacagccctgagattctacctcataccagtcagaatggctaaggtcaaaaagtggaatggatacagaaaacgtggtacatttacacaatggagtcagctattaagacaatgaaattatgaaattcttaggcaaatggatgaaactagaaaatatcatcctgagtgagttaacccaatcacaaaagaacacacatggtatgcactcactgagaagtggatattagcccaaaagctccaaatacccaagatacaattcacagaccacatgaaactaaagaaggaagaccaaagtgtgggtgctttggtccttcttagaaaggggaacaaaatactcataggaacaaatatggagacaaagtatagagcagagactgaaggaaaggccatccagagtctgtcctacctgggaattcatcccatatacagtcaccaaacccagacactattgtgaatgccaagaaatgcttgctgaaagaagcctgatatagctctctcctatgaggcctgtcaaagccttacaaatgcagggggatgggggatgggataggggctttccaagagtggggaactgggaaaggggataacatttgagatgtaaataaagaaaatatccaataacaaagagagagagagagagagagagagagagagagagagagagagagagagagagacacgccatggctgcatggaatgtgacattaagaaatacatcctggcttagccaattgactcaggaaattaccatttcacttcaggaagagtgaagtcatgaagacacctcccccctctctggttttagtagctatctaaagtatatctttctctctggtttataaggataaaactcagagagtaacaaagcaatagcaatactgtatccccgttcttccatcccaaggaaataccatgtgcatatataatcagaaaaagctagctgtaagattctagtagtgtttgaaagctaaaatgtgtaaccttttctttctaggcctttgaggatatatatattgatcagcgaaagacgatcaaaaccatgctggagtatgctgacaaagtcttcacttacattttcatcctggagatgctcctcaaatgggtggcctatggctatcaaacatacttcaccaatgcctggtgttggctagacttcttaattgttgatgtaggtattttccatgtcgtgccttttgtttgagggagtttgtcttgtttgtatcccaattctaaaccaatgaatgagtctcgggccactgtgttgtgatgacaagataggctccccagccctaaactctctcacctaataatagcatagaacttacaagttctggactaatttactaactaatttaaaaagaggagataagatctagtaggaaaaccaaatttaaacaatgatattggtaatatataatgcaaatgatagaaaaagacatttaaaaggtatggtgtctattctgccatgttataaacttgaccgactccagattgggtcttcatgcacttattcattgatgttcttccccagtggtaaataatagcaagatacagaagggatggtgagcactattgtaatcttataaatctttcttatctactggaagattttacggagagatttttaagtctattacaagtaaggttacagagggtaggggaaaacaaacctacttaatgatgaggaagaaattttcttttatgtaaatgagatttacagagtccttgatgtaccaaatatccttatggagaattttagttatgaatagtgagaatttgagaatagtgagttatggagaattttagttatgaatagtgagaatttgagaatagtgagttatggagaattttagttatgaatacttaaaaacaagtaacagggccactcagtgaggccaaaatggagtgagcaaattgatggaaatctcaaaggagtcatcagttttacagaaaagagagaggaaattaatcaataggcattagcttatgctttcataactccaagtggggttaggacttggtattttgcaaattctttactcttgatattcatttatgtccttttaatattttattacttttaaacttttcatacagtgtaaatttttctacggtgtattttgcattatattgcttaccttcccccaagtcctcccagatcctcctgtcttgatatcctgacaactccattctctttataaaaagaaaaggaagaaagagaaagaaagagagagagagagagagagagagagggagggagagagagagagagaaagaaagagagaaagaaaagaaagaaggaaggaagacagacagaaagacagaaagaaggaaggaaggaaggaaggaaggaaggaaaaaagagaaataaagaaaaagaaagacagacagacagaaaggaagaacgaaggaaggaaggaaggaagagagagaggaaggaaggaaaggaaaggaaaggaaaggaaaggaaaggaaaggaaaggaaaggaaaggaaaggaaaggaaaggaaaggaaaggaaaggaaaggaaaggaaaggaaagggaagaaaaaagaaaataaaaaaataagcaataaaacacaagcaaaaacaaacaaaaaactacaacaacaaaaaaaaaaaaaacgaaaacaaccaaccactaaaccataataacaacaaaataagagcacacaaacaaaaatcaaattcgagaacatttgggtagaccagctacttctgctcttgcagactgccctgagtgtggttcatatacccagtgtcactcccttgaggaaaactgatttttctctcccagcattatcaaatagcatcttggttaggggtaaaaacttcctgctcacctcccctcccccacgctacaatttagtccagtttaaattttccacaagtcttgtccgtcaagtctgtcaccattgtgagctcatatgtccctcaccctgttgtatctggaagacactaattttcttggagtcactccccacctctagctcttaaacttttttaaaaaaaaattttattagttattttatttacatttcaaatgttatcccctttttttttgtttcccctctgaaaaccccttatcccttcccaactccccatgcttaccaacccacccactccagcttcctagccctggcattcccctacactggggtatagaagcttcataggaacagcgtcccacaaggccatcctctgctacatatgcagctggagccatgagtccccaccatgtgttctctttggttagtggtttagtccctgggagctctgggggtcctggttggctcatattgttgttcctcctagggtgctgcaaaccccttcaacagcttgggtcctttctctaactccttcactagggaccctgtgttcagcccaatggttggctgagagcatccacctctgtatttgtcaggcactggcagagcctctcaggagacagctatatcaggctcctgtcagcaagcacttgttgacatcccagagactgctccacctggggatccatcccatatacagctaccaaacccagacattattgtggatgccaacaagtgctcttaaactctttagcctcctcttccacatagctctctaaggagtgagaggaggggtttcatgaagatatcctatttagtactgagctttccaaggactctcactcagcactgtccagttgtgggtccctgttaaccatcacctactgcaagagagagcttctctggcgagggttaggtggggcagtgatctatggataaagccgttcatcattaggagtcactattgctgtgtttattcagtagaataacagaacagctatgaataaaactttatctgttgtattagttgataactgcaaattggacgaaagcggtgttattgattcattgttatttgtattgtacaggtttcattggtcagtttaacagcaaatgccttgggttactctgaactcggggccatcaaatccctaaggacactaagagctctgagacccctaagagccttatcacgatttgaagggatgagggtaagaaaaaggaaaactctgcagcgttgtatattgtcaaagctaggctgagttcaacttaactaacgaaaaacacgtgcatgcaaaaggaatggcaaccctttgcaaacttgctactttacccttttctctgttgcatatttacttcttggtgatatgcaagagaaaatcggcctctttgaaaatgatttaatatcatttatctgctttgctaattaaaatgaccttagttcataatcgatcttgggagtttccttataattcctaatacaaagggggaggggcagatactctcttaaagaactaagttgagtcatgtaataattacctagagataattttgtttcatatcgttctcctctatgacagcccatcagtacttaagggatcctatggaaagtaatgtgaatcacaaatgtgtatgaatacaaaggaaaaaatgaagaattgttaaatgttttgtctttacaatgccaaaattctcattatttgaatatatccaagggcagatattaaccattgactggagtataataatactgcctcaactgtaactaaattaatgacattgaataagtaagacactaatttaattactataaatacatacacatcttatgacaatacagctgataaggaaaaagaacatgtatttttattcattgccatacatggctcgtcaaccttaacttaaacctcggttctcagttacacagagttttatgttgctcttttgagcaaagcattattcccctctccataattcaacacgtatcagatttttgcatttattggctcattgttatgatgattatttcaaagcattatacaatcatttatagaagatgtgccgtgtgaaaattatttttttattaagatccaaaatttacgctctttaaaccaatcagatgaaatgtataaggcaaagagtgctcattgtcctgacacttacaaaccaaggctccaacaaacaggctcctctctgcaccacatagagggctttcagcctgtgtccccccataaaaacctattataaattttattatactatactgtaagaaacctgtccaatttttaatttctctagcacatatgaaaacttctcttcagtagatccaagtaagcacaaaggagctttgattctcacacaagaaatcacatttgtattaaaaatgtatcataaattttctcccaattatgcaaaacttaaatgcttttccaattaaaagagcactttcgtttcagaatagcaatttcagttgtcaaggaaaacattgtttttatacattttatataaaaatacatgagctaaatttaaattcacatttttcaactttttatggtttttttatgtttctttttctttcgcattttttaacaatccagccattacctctcctccctgtccccctcctatagtttctcatctcattcctcctcccccttgcctctgagaggatgctccctccctcactaggcctccctcttccccagggcttcaagttcctcaaggattatacacatcttctcccactgagaccaggccaggcagtcctctgcttctgcccagcctgtgtatgttcctgcttggtagctcagtctctggaagctccctggggtctgggttagttgggactgctggtcttcctatggagttaccctccccttcaacttcttcaatccttcccctaattcaaccacaggtatccagacttcagtccaatggttgagtgtaaatattggcatctgtctctgtcagctgttgttaggggctcagaggacagccatgctaggctcctgcctacaagcagcacaccataacatcagtaatagtgtcaggcctttaatgaataatgctacgtatataaggttgttagattatacttcaactttgatcttttagaatattattaaatccagtcgtttattttttatatgtaatattgactttccataacaaatgagtctattttccttttgtgtagaaataactttatcaattattgttaataatgcttttgtcaattattgttgatatgcttctcttttttaaaactggagtcatcaaaacaaaaattctgggtagatattacgaagctgactccttggtcagcttggcacatagtgagaccacaaatatctcaaggtcacggcaattcctcaccaccagtttggcattgtgaagtcaaaaccaaccttctgttgattctggtttttgtatttctagtatgagacattttctactttgtaagagtatataactgtggatggcggcgaggttgggcatgatgatgattgtaagcgggaagtgagctagagtatcttcagaaactctcactttatcctccttggcagcatagaaccgcaatcgctgtgtccgagtgtcaaccaggcagtcattttgttttgggttttttgtcactctttcaaagcatgtgcttctacgcaacactaccaaacacagcatgctgcatagtgcttgagaaggagttagaaagtaacaaacgagttatcatcacgttgccctttgtgttttgcatgtcttatgcacacttttggctgacagcttttgaacatttattgtatttcaaatttccagtccaaatttttttttcaacttgtgaaattgaacggaatgaaccgatgtcgtgaatacctagggtcaaataaaacttgtatttaaattcgtagttttaatttcccaagctgggaaaatcgtaaaaaccttttccaaagaactcaagtcttagttgctagggaaacaggcagtgagcatcatatacaaaaagtaacaccaaatgttctgtcatatcagctttctaactaataataaactatatatttctattttatataggataatcttgctccaacttggatggggtggagcggtggttcctcccctcagccctttattatgggtactgtattaccccttttgctacctttaatccttgcactgtgacttatgtgtagtgggattgagggagggagtgggaagggtacaattgcaccacagtagggacaatacaggatttatttccaaatccactacttttaatgagcttaaacttcttttgggaaaaaaaaagttatctctgacttaccatctgtggattataaccccagaagtacatatctttattacgtttcactgaatatgatttagctatttatacttcattgtccatttatggggaaattactcaattggtgagggtgggggaccctggtgtaggatgctgatgaaaacgttttcatttgtcaagctcatggtagtgacagagcatatagtccttattttttcaacacactgctctggtccctcaatgggccagtcacattccatcagttgatcgttgatgtgtgcgagcagtggctaaaggtacaacaggccaggtatctcagggttgccaatggttatgatcattctcatctttattgcataaaaatgtgttatttgcagaaagtagcaaggcaagatccctgtgaaacaagggaatacaaaaaaaaaaaaagatgtgctttaagttataaaaccaaaacatgtgaaaagtcaacttcattgaagtataaagaataggatatgcatgaaaaacaaaaaaatcatgagcactaagaaattggtgtataagccaactccttgtaagctaccccaattaacttcccagaatcttagaaggcatcacagtgcaccccaaaataaaaagccaaactgacacttctgcttcctcttaaaatgtaggagtcttggataagaaagataatttttattgtttggaagaaaaaaaaattgtttggataattgaggcatttatctatcaaaaatatttatcttaataaaatttcacaacactgatttagttgttggcttttctaaaaattttttatatttcatattaagaactcatgatttttactttccattttttaaattcttattcacatatggtttttctctatttcttagaaaagctatagaacccatggtttccggtgacttaaaaaactaatctaaatgtcttcacttagatgatactttcaaatgcactgaaatttctaatatcaacaagaatatttgcctggtcctaatttttcactgatttaataaaaagtatgaaccctaaagaagaaatagacttgaagaactggttgtgtgacaatacagaaattctgctgggagatgtccttttaaaacattgttagaagagacaacctctacaatccacccattaagcatacttctctcttagacatctccttttatgtaccttataacctcaatgtgttctttccaattgacttagaccaacacttcccagcgcatcccacatgggagccaattactgactctccctagagactgcaaagaattaatattgtagaaccaagggatggttgggctcttgggagaggcaatccgtttgtgatcttttgctctggatgttaatgaaatcgcaactttaagtggattttcagtggcaaatcctctgatcctatgccaaatgttctctaagacaaacatccttgtaaaataaatgtctcactgggccaaatctatggcaaatttgcacgttttcctgaactgcattcctatatagtatttgccatcctgaattcactaatgggcattacttttaattcaaaaccagtcccttcttcaaaggaaatctctcccatttattacattatgcaaactgctttcttatgcagtggttaaatccttagccaggcaagtatgaggactggaatttggatatccagaaccctcagaaatgtcggatgggcatagtagcttacatgtaattccagagctagaaagatgaaactagccctgtcctcaagctctgaattcagttggtgcaataaataagaaagaatccccccccccgacccctacatctctttcctctacatctatacatgtatttcccatacagctctatgcttccacatatatgctcacatatgtgcatgcacacttgcacacatatgtacacttgacacattgaagcatcataatctaacaattggaatataagaaaatatttaactttcacacagagcagtcagagaaaaccataagaggttgaaactcttgaaaaaacttgcaggataaacagaaaagagtatgagatgccaattctggtctactttctgaacctaacttgttttaccttcattagtctaattttccaaatgaaccccaagcaccaaattgtccttattgctctttccagtactaaattataattcctcaattcgaaggcaaacactctcatctttgttaaggatgtgtagagttagactcaataaacgacatgtatatttgagctaaagcctggaggagggagattatttcaagggcaagtacttggcagggagtttcaaagaaagaaggctctcaattctcagactaacaccttagcgtggagtgactgtcacagaggagggtgaagtgtatgtcaccagttagggaagctgaaaggggaaatgtatcaggcttgtgagaatccttcagcagccaagtctagcacctgagcacaatccccagaactgaccccacatggtggaaaggagaggaataattcctgcaaattttcctgtgacctccacccaagtgctatagaaaatgcatgcatgcccacatgcacacacaaataaacatagttgcaaactgttttgaggaaaataacctcacaaactgtcgagtgatgtaaatgccaaagaaagagaaatgttttctaatggctagagaaccattaaggaatttttcaaaatgggacatgggatagataaatttagtatccatactgaaagaaggcaagcaaataaaatctgataagaatgtaattcttagtaaccgaggacagagcgaagatagagaacagggccaatggccaaggtggaaaggtttgaaggaagcagcatgaaacctacagacttgtaccaaaatgttcagtgtcatgagtgttaaaagtgaaaagcttgcatgttagtatggatttcatatcctcggcagacagagcacctcactgtgagtgggagatgaagtattcagaaatgagaaacaactactcaatttcagtgttcatatctcaaatccaataaacaactttaggggtacaattttttaaaaaattacattaaaatgtttttaaatctcttcttaataatttaaaaattaaattgaaaataactttaaaaagtaatatatacaggaaagcctgtgtgctaattttttagggaggccataaagggagatagttgctcattaatttctacacatcagcctatctttggcttctgccttgatagcgcactctgaattatcttcttcatgttcatccctcatctttattgttactggtttcatttcccttggccacatagcccactattttgtattccccaatggatattgttccttacaaagttcagccagggctcagaagtacaaggaattggctcttatacttctgtcagacaggcaaaaacttctaaaattatactataataaaaatcaaagagatgatattcataattaaactaacaaaagtggcaggccccccctcccccaacatgagtagaattaatctgacgtccatgttcaagtctgaaacacacttgccaattaagagcacattagggccagcctttatctccctcttagttactaatgtgcagttcaatggtgagctatagagaaggaagccaagactaccatatgtcaaatataaaaaaaaaaaatcccattttaaatctgtagtcccgaattaaggacaagagagagggaaatatctttgacattagaaaatggagaaaatattttagcacaggactttactcagtcacatcagagttgataagtacgtatgacatccctctttttcctgttttcctgagaaaatgatctctctagtgtttcatttaagataagtttattgaattaaactcagtaaatgaaacaactgacatgactggagcttgaaataaacgatgtgatgatctactgaaatacatgatgctaaattgtcttgcttcttatgcaaaaactactattagttatagcaatgcatggataattaaggccaaaaatatattagatgttaaaaatagttttatatttatacatctgaattttaatttatatttaaagtatattggtccaatcaattcatgcccaaatgttttagttctattctttgagatactgttttgttttgggatttttttttatgagctaatctcttgcctaggagttcctacttctctctcctccttttattttttctaataaactacacatgtgtcttcatccaggagctaacttctcccattttgcttttcctttagcaccttttttatattagatttctttcttttctccatctctttgcatattgcctatatttcttttcctaagcataatatttaaaaaagactgagttttatgttaagattatttctgctttgctcttacacagataggataagtagtcttgatagaaaataaatcaatgattcctagggggatgtctttttgcttttaatcaataaggattctgacttctctttctctccatttgtgtattaggtggttgtgaatgccctgttaggagcaattccatccatcatgaatgtgcttctggtttgccttatattctggctaattttcagcatcatgggcgtaaatttgtttgctggcaaattctaccactgtgttaacaccacaactggtgacatatttgagatcagcgaagtcaataatcattctgattgcctaaaactaatagaaagaaatgagaccgcccggtggaaaaatgtgaaagtaaactttgataatgtaggatttgggtatctttctttgcttcaagttgtaagtgaacactattttctctgaaaatttttattgctcagaataataacaaagtgattattgcagctgttattttgttaggaagaaactttataatatgttttcagcattatcatctgttagtacaaatttgtgaatccagataagtgcacagaactctcaaacatgttctgctactaagtaaaaatttcacacttgactatttcgcccacctctctgttgatgagtaagttcatagaaataggtcaaatcacttttctgttcctgtaggaagtgtgtgatagacaggttctataattggtctaagccactgacacatcacccatctctctctttgatgtgaaaagaatttccgttttctgactaagatacattagtctctggtcacactcccacattttccaagagtcaatgatttaaatacaaagataaatcacaaaaatatgcgaggcttcctcattgactatggaatatatatttcaggtcctgtagcctcaaacttgtgttctaagacacatgacaaatgcctgcaagaggttagaggaggaatccctcatcaatgtgcaaatgacaagtgtcagtcctattttgtcacacttttattatgttgttaattatatttacacaatcttttcaggggttaccccatagaaaatgaatatagtgttatttctgaaaaattcttaaaagaatagcttcaggttttttctttatgcccatatgatccatacactgtaagcataaagaaaaaccttaaatactgtcaacttgatcacctagaagacgagattcccacagaatatcgcagatctaatttgttcttgctgtagacactgccaagtccacttacatttctaggactttcttccaaattgtaatagaatgttaagcttgagagttactttagagctggttggaaaataggaagttatatcagatcgccattttgttggctttctatctttttttcaaaatccaaggaaagtttgttttgaaaatagtttaaaatatttctttaaacatgtacagtaaggatattagtaagcatcagttttgaaatttaacccaaattaaggcatgcatgtcctttccaacaggccacatttaagggctggatggatatcatgtatgctgcagttgattccagaaatgtaagtattttttatatccaatgtcttctggaaatattgatttggtgcttaaattagcttatgaattagccaaacaaactaactccattttagttaattaaaaaataggaagacagtatattgagtttgtacttaacacagcagtgtctatcagagatattgtactgagttcagaaaaaaaaatggaagtcaaaaaatgaaagaaaaaattcaactctttcatttaataagcaacagatattctccaaagtgtttctatttgttgtataaaggacatagaagaatatatgactgtcttcagtagctgaactgtgtaggcaggctccatgctttgaagcctgaaaggctaaagatgcaggctaaacatcttagaaatgtgtcatagtcttttctaaatccatcctttataagaacagcacccatgcattcactggagtgtgtaccaacactgttatcacttctactttttaattccatacttttcttagctttcagatgtaccaacactgttatcacttctactttattgattccatacttttcctagctttcctatgtaccaacactgttatcatttctactttattgattccatacttttcctagctttcagatgtactcaaaaatcacaggcttcctctaagattgatctccaaaactgtaaaatctctgttttcttctttcctgttacttggttgttttttgttttttgtttttgtttttttattcttgttcttgttttggttttcttcggtaacccagacctctagcttactatggagacaatgataaatttgaaatcgtcctgttcccctacaaacttcttgatcacctctggaattgcatcatctcaccttgttgcatagagtttgtcttagatgttcagttaattttagctgttgtctttaaaaatagaagactgcagtagttacctatttatactaaatattgactccattgttatgcattgagtgacagccctgtgtctatttttataactgcaagtctgagagtcagtcgtcaatggaggattttataaaataaatcattgaacgtgtctattcaatccaaaaattttaatgactttgttactggtagggcaagatgccagcatccaaatgaatacttcagttccttcatttgctatataagttcatacacaagtgaatcttgactataaaagtgtcctctctatatttataagagctaatttttcactaactttttctttggttgttttaaagacaggatgttgcattgtggcccaggctagaatagtactcaacaagtctccagcctcagcttcccaaattctggagttgcacagacataagacaccacactagtattttcaaattactgaaaatcaattaacttaaaacttaccaaatctaaagtctattatccaaataacagaatgtcagttatactatgtaaaattatgcaattagtgttagagtgacgaattgcatcactttttacacaaaaactttttgcttcctagtttcaaggatggtttttggttttttgtttctttgaggctgttgatatgggaataaaatacactaaattatttctaagaatatatatagccttctactcttgtgtagaagcaccgacttgctcccagtattcaaactgctttatcagttaacaaattccctccgttggcaggttgaactacagcctaagtatgaggaaagcctgtacatgtatttgtacttcgtcatcttcatcatcttcgggtccttctttaccctgaacctgtttattggtgtcattatcgacaatttcaaccagcaaaagaagaagataagtatttttacaatttttcctaacacaaggaatcagagtaattaacattctatttgaattggaattcaagatgagatcttgaattcagaaaccaaatagctcacgaccttttccctatgttttatccttaataactcctcactcactcccctttataaggtattcgcagctaaaagatcactagaggctggaaaagcatgtggaaaggtgagggaaaaggggttagatttgatcagagaacactggcatgtctgtactacaatatcacactaaatcccatgattatacatagtcagtaattatccatcatttaaaatacggttcagtgtatatacttcttttgtactcttttctacctgtacttctttcttctggttatttaattaatagcaaccacttctagttctaatttgaggaatttttgttttatctattatcattcttaaggcatgtttgattatttcaattatatatgtgcactaaaggaagcatgcaaatattatcactcagtagtaacaatggtccacatttcatctttgtctgctcaaactctttccatgaacagtctccttttttgaactacttctcatctgtttttttcttttaagatggtctcacttttcaatctaggctgacctgatctcactatatggccctatgtgacctaaaacaagtagcagccctgtatccttctcaaataaattggaactatagtcacatatcacgttgtctgaccctgaaatattctgtgtacatatgtggctctgtgtgtgtgtgtgtgtgtgtgtgtgtgtgtgtgtgtgtgtgtgtgtgtgtgttgtgtgtgtgttgttgtattgtgttgtgttgtatgtgtattcgtggtggtgtgtgcaagtgtctgtgaggatgaatgtacacatgcatgtaaaagccagtgataaacattagtcacctttctcaatcactacccaccataatttttaagacaggagttctcattgaatctcctcttcaattcagattgatggctggccagtgagattaatggattcatcttatctctgcatccccatcaccatttggtttttgttttaataacatagtgccatgcccactttaacatgggggttgggatctgagcttatattcacgccaatgtgtaataaatttatcacttcagccatctctgaaatactttttaaaattacagttatttattcatgccacactgggtgtgtggtagtcagtagacaacttacaggatatggtctccctccttccaccctttgagtcccagggattgaatccaggtgatttggattgtaagcaaatgcctatatgcactaagccatggtcccagccctgaaatgtttcccaatgtacaattttaaatacttttatcacagagatttctcagtttgttaaaagtgttcatgtttatttatcataacttggccaatttcaatctatctaatctttagtgacttaagtaattttttcctgcaaaacatttgagtgtttaaatcttttcttacattaaattccagagtgaagtatattgcctccttgtagttaaataactagtcaactgaataagctatgtgagcatgaccttgctccaccactctcacagacaccagacattccctttagttgttaatatttgacatctcttaaagggaaacaaaatttaactttattttttgtgcattttatggcaaatagttttggatgtatccaagttcatcccagccaattatatttctttatgtcccatgatttcatactatttgttcgttgcattcattttttatttattatgattaacataaaggagtattggatttgtcaaaggttttctcagcatctaatgtgatgatcatgtgatttttttctttcagtttgtttgtatagtggattgcattgatagattttctttcatatattgaaccatccctgtatccctgtgatgaaaggcctacttggttgtagtgaattatgtgtttgacgtgttcttcaatttggtttgtgaatattttatgagtgttttttgcgtagatgttcataagtgaaattggtctgaagttttcttcctttgttgagtctttctgtggtttaggtatcagggttactgtggcctcatagaattaattaggtagtgtttcttctgtttctatttttggaagaatttggggagtactagtattacactgatggattcctacatattgaaccatccctgcatcactggtatgaagcctatatgatcatggtgatgatgtttttgatgtgttcttgaattgggtctgggagaattttattgattatttttgtatcaattttcattgacaaaatggttctgtagttttctttctttgttgagtctttgtgtaattcaagtatcagagtgactgtggccacatagagtgaactaggtagtgtttcctctgtttctatattgtggaatagtttgaggagcattggtattctggtagaattctgcactaaaaccatctgaccctgtactttttttggttgggagaagtttaatgcctgcttctatttccttaggggttataggaatgtgtagatggttattcttatcctgatttaactttggcacctggtatctgtctctatttcttcctgaccaagttagtgatcattgagtatagagttattcagtttccataagtttatgggctttctgttatttctgggtttttgttgttgttgttgttgttgttgttgttgttgttgttcttttttttttttaagtctagccttaatccctggtgacctgatacaatgcaaggggtaatatgaatcttcttgtatctgttgaggcttgctttgtgcccaattatatggtcagctttagaaaatgctcatgaagtgctgagtagaagccacattctttggttttggggtggttatttagatatctgttaaattcatttggttcataacttctgttagtttcattgattctctgtttagtttctgtttcaatgacctgtccattggtgaaagtgggatgttgacatttcctactattattgtgtagggttcaatgtgtgatttgagccttagtaaagttccttttacaaatgtgagtttccttgcttctggggcatagctgttcagaattgagatgtcatcttagtggattctccctttgatgagtatgaagtgtccttccccatctcttttgccaattttggttgaaagtctattttattagctattaaaatagctactccagctggtttcttgggttcatttgcttggaaaactttttttgaggcctttattctgaggtagtgtctttttttgttgctgaggtgtgtttcttgtatgccacaaaatgatggaaattgtttacgtgtctgttagcctgtctttttattggtgaattgagtctactgatactcaaatttatcaatgaccaatgattgttagttcctcttattttgatgttagtggtggttctgtgtgtgtgtgattctcttcttttggttttactgtaagatgattaatttcttgtgctttcttgggtgtagttactcttcttgtgttggagtttttcttctagtatccattataggatgtattagtagaaagattctctttaaatttgggtttgtcatagaatatcttggtttcttcatctatgatgactgaaagttttactgggtataatagtctgggctgctatctgtagtctcttagcatctgcaagatcttctgacttttagaggttcgttgggaagtcaggtataattctattaggtatgcttttatatgttacttggcccttttcccttgtagtttttaatattctatcttttattctatatatttagtgttttaatatgtggaaggaaaattttcttgtctggtccaatctaatgatgttctgtaagcttctatagtgatccctttctttaggttaggaaagttgtcttctatagttttgtttaagatattttttagccctttgaactgggaatcttcaccttcttttatttctattattcttaggtttagtcttttcatcgtatcacaacttcatgaatgttttgagttgggaacttttttcatttggcattttcctttatcaatgtattaatttttttctattgcatttcctctgtctgagattctttcttccatctcttgtattttgttggtgattcttatgtgtgtagtccctgctctctttcctcagttttatatctccagggttgcttccatttgtgttttctttaatgcttctaattctatttttaaatctgagtccattttattcatttctttcacccgtttgattgtatttttcatatttctttaggggatttatttgtttcctcattaaagatttctacctgtttagttgtattttcctatattctgcatatgctttatttatatcatctttaatggcctctattgtcttcataagatgggatttaaggtcaagtattgctcttcaggtgtgttaggatatccagggcttgctgtagtaagagagctcagttctgaaggtgccatactgcactggcttctagcgattattctattgttctattgagcttgcctttcgccatctatttttttctctggtgttggctggcctgtaagtccttgggagtagtaactctcagagacttgttgtcctagatagctaaagccctccccagaggcaagcagagctgttggctgaggtgtagggtatagatccccaattgcaggtagaggtgtggactgaaacatggagctggttgttctggtgggagaagaactcccatgaggcagacaggttgtgaaggagagctcagggtgctggtccctgactgtaggtggaagtgtgggccagaaggaagatggagctttgtgttttaccatgtctgggcttttttccttcaataattataataataatgccaattttagttatcttaaaacttaagtattatagtcaatacagtgtacttttaattttcaaattcaaaataagtcacaaggataaccatgttaatctcttgaaatctcttgttataatattgaaaagttatgagggaatggatacaattttaaccagctttgatttttttttctatactttggaggtcaagacatctttatgacagaagaacagaagaaatactataatgcaatgaagaaattaggatcaaaaaagccacaaaagcctatccctcgacctggagtaagaaatatcaaatgaaaataatataaagcaacaacctgaaatgagttttgtttctcaggggaaatcaactaccattcccagcacaaaagaaaaaagaaaaaagaaaaaagaaaaaggaaaaaaaaaaccttttgagtatctctaatctatgttttgatattttttttaaagaccaaaaagaataagaatatacagaaaatgcaaatttgtctccaaatgacctacaactacacacttagaatccaaactacgtagactaatgtggtcctcacaatagttgtgtctttatgactttacagtagctgcagtgcaatgtcaagcaatacagaagcatagggtggggctaatattcatttactttctgattaaagaggtagaaaagatgctcaatggcatatcatctccacttgtaattgttgtttaataagtagaaatatggaccattttagaggaggtgagcatcccagtaatgaatatgtttttgtatggctctaaaatcagcaattgcttatgtcggcattattacaatttgtgataataaataaccattctcatatttccaagattcatctacaaactataaaggagaaagagcccttatcatggttatttttatgacagtgatttttttccataacttatttcttttattcacattatatcccaatcacagcctccctccctcctctcttcctagtcccaccatcacaacaaatccttcttcctattgtcccctccccttgtcctcaaagaaggggatcccccttttataccactccaccatgggacatctagtcccatcagaactagacacatcctctcccaccaaggactaacctggcagtctaggtaagaggagggggctccagtgacaggaagcagagactgagacagcttgttagggaacccacatgaagacaagctgcacacttgctacaaatgtgtagggtacctagatccagctccaccacgctccctggatggtggcccaggctttgtgagtctccacggtcccaggatagttgacactgtaggtcttcttgtggtgtccttgactatggcagtgatcttaagcttaaaatattccccatgatgaattacaaaacagaatcttagtgaatgtttctattgtactagccaattttgtttgtttgtttgtttgtttgtttgtttgtttggaggcagggtttttctttggagttttggctgttgtggaactcacactgtagatcaggctggtcttgaactcagatccacctgcctctgcttccccagtgctgggattaaaagcatgctctactactgtccagttcaagcactactttctttctttctttttttaatatttttattaggtattttcctcatttacatttccaatgctataccaaaagtcccccatacccccccccactcccctacccacccattcccactttttggccctggcattcccctgtactggggcatataaagtttgcctgtccaatgggcctctctttccagtgatggccgactaggccatcttttcatacatatgcagctagagacaagagctccggggtactggttagttcataatgttgttccacctatagggttgcagttccctttagctccttgggtaatttctccagctcctccattgggggccctgtgatccatccaatagctgactgtgagcatccacttctgtgtttgctaggccccggcatagtctcacaagagagagctctatctgggtcctttcagcaaaatcttgctagtgtgtgcaatggtgtcagtgtttggaagctgattatgggatggatccccagatatgtcagtctctagatggtccatcctttcgtcacagctccaaactttgtctctgtaactccttccatgggtgttttgttcccagttctaagaagaggcaaagtgtctaatgagaacattttccccattgatacttagtgctacacaagtcaagtactgcacagcaggggtattttatggtactaaaatatgcttaggattcttcagtggcccaaagaaataagcagaagatggtgtactgggcctaacctcagctgactacacatgagtagcgaaggacatgtgctccctttgttatagcatagcaagcaatgtcttttcagtctgattacccctgtggagactcatcattatttgtctgacaatatcagcaaagaccacttgctgggaacaaattaatgttgtcaaggtgaaatgtaaatttattttattttattttattttactctttgagacagggtctctctgtgtagccctgactgacctggaattccctttgtagaccaggctggctcagaaatccacctgcctctaccccccaagtgctacttgcccagcaggaatgtaaatgtttggtgtttcagatgcatccttgaaatcacaaggttcctttgataattctctaggaacaaagcaaatttaagactaatatttattacgtttataaatgaccacattgtctagctgctcattggaaagtactacagccagtgagccaaagtttggcaacagcaaatcaaacagatttcttacagttctgtaaccttgatgtgttgaaattcactgtgtgtttctgtaaagagtgtgcagggaaagaatctacagcaagggattgatgtattcaggaatatgcatgcctcaaaacaaaaatttaattaactagatataaaaatgttctacaaggcaccaattacaaatatttgtcccaataggtaggtactatgtacaatactagaaagtagggaatataggaagtatttaatgtatatgctgtctaccaggaatctcccgcattgctgaaagtgtctttgtttctctgcagaacaaatttcaaggaatggtttttgactttgtaaccagacaagtgtttgatatcagcatcatgatcctcatctgtctgaacatggtgaccatgatggtggaaacggatgaccagagcgattatgtgacaagcattttgtcacgcatcaacctggtgttcatcgtcctgttcaccggcgagtgtgtgctcaagctcatctcgctccgccattattatttcaccattggatggaacattttcgattttgtggtggtcatcctctccattgtaggtaagatctgtgcaaagtctggcaaattcctcctgataaaagtggaggttgaaaacaggaatttagagtggaaggcatctcactactcaaagtcaatatgttggcatgtgtttttgagttgtatttgtttttcttccaaaattaggcttctaatttatacaaatattttatcccattgcatcacatatttatttgaccattaataagatctcctttattgagatatattaaatagattgatgtggtgaaccaaaatttaaaaagtgcttttaggtggttgagattaaacactctatacgctatccagatcaatgattctcaatctgtaggtcgcaacactttggggattgaatagccctttcataggggtcacctaagaccattagaaatcataggtatttgcattaagattcatagcagtagcaaagttacaattattaagaagcaacaaaataactttatgattggtggtcaccacaatatgagaaactgtattaaagggttgcagcattaggaaggtttagatccactgatctagatgattctggtaagaggggtactgagaatctggtctgaaggtcaggaaagacattacattagagatgtagttaatggggtcaccagcatgcagagtagaacatggatagaagtaaatgggaaatgcaagtagactaacagtagtttcttgtggctttcatacagaaattttcaatatttccagaaggaaactggtagcatggaacatctctttcttagtcctatcctacaaattgtgtagtacataagagcctcatgaaggtaactcatctcactgcagataccaacaatgcttcagagaagtgaagaagtccctgtatgggaacttcattcctaattaagatgaaatcattgcaaattttcaagcataggactcagttggaatgatctgtgtgctattcccccctctgaataaataggccttacatctaaaatccaacccatatgttatatgtaatttttaattccaacaataatcatgtaaatagcatcaaaaagagagaggccgaaagagacagagatagagactgagatagagaaggctgactaaagagaatgatgcttttgtgctttgcatacaatgatgcatttgggtattttggttggttgaataggtaggtttttgttgttgttattgctgttggcagtggtggtggtgatggtggtggtggtgatgatggtggtggtggtgatagtggtggtgatggtagtggtggtggtggtggtggtggtgtgtgtgtgtgtttgtgttggaagtactctttaatggagtctttatttaaaggacagaggggggagctgagaaatgcagtggcatctaaagcacttagtgtgcaagcttgagaacgagagaacagattcccagcaccagtgtaaatcctggccaagggtgacaccttataaagaaggctcttggaacccctcaaattatctggcaagctaccagagagctctgtgttcaattgagaagtcctgagcacagagactgcaatggaggagttagggcaaggactgaaggagctgaaggggtttgcaaccccacaggaggaacaacaatatcaaccaaccagagctcccaaagctcccagagctcccagggcctaaaccaccaaccaaagagtacacatggagcatggctccagccacatatgtagcagaggatggccttgtcattcatcaatggaaagggaggcccttggtcctgtgatggctcgatgaccccgcaaaagaaatgctagggccctgaggtgggagttgatgggatggtgggggaacacccttatagaggcagggggtttgggaggggaaaatgagaagggggataacatttgaaatgtaaataaataaaataaccaataaaaattttaaaaattgaaaataaatacataaagtagaaagcaatcaaagagactcccagcatcagcctcaggtaagacacacacacacacacacacacacacacacacacacacacacacacactcacgagagagagagagagagagagagagagagagagagagagagagagcacatgccacatgaacatacatgtgccaaaaaacccaaaatcacagggagaaaaaaagctgtaaaaattaaaaaattgagattttgctacaggagaggaaaagaacatcaaccctggagtgttttgcaaacacacaggcacatgcacggacaattctgtaataatattagttcatatgaggatgaaaacagcatgaaacactccaagacttcagattagtgaagtgtgaaaaagaaaagtttggttcaacaagtttagagaaaaaacatcatattcatatattgttggctgttgacattgaagcaaattttcgtatttatttctgatgagattttaagttaaccatagcatatcaatactcttcgggccaggcgtggtggtacatgcctttaatcccagcacttgggaggcagaggcaggcagatttctgagttctaggccagcctggtctagactgagttccaggacagctagagctacacagaaaaaccctgtctcgaaaaaccaaaaataaataaataaataaataaataaataaataaataaataaatgctcttgggggggcgagctgtactatgattcttaaattttaatctattgctgaggctgagtaaaagctaaagttaacttttattatacatcgtataattctataaacactcagttttccatatgtatgtattaaatatctgaaaattcaaacacagatcaaaagtatttttaagaaccatgctcctctcctgaccatttttaatataatgaacggtagcaactgttctgttttggattttttgtttttgtttgtttgtttgtttgtttgttttcttgttggttggttggttggttggttggttggttggttggttggttttaatttggtttggtttttcaagtcagggttcctctatgtagccttggctgtcctgaaactcgttctgtagaccaggctaccatcacactaacagagatcccctccctctgcctttgagtactgggatccacctccactcagccttacttggtttttgttgttgttgttgttgttgttgtttagttaggattggatttctggaatatcacagcaactcttcacatggtactttcattgttaaatgtgatgaacattttaaatataattcaaaactttcaataaaatggtatagacaaattgtatgcaaatgtgctgccatatgatataaagggctttcagtccttgaattttgatgttctaaggaggagaggtgcatcctgggacaaatcagctgcagatactggaaaatgattggaatatatttgtaccatacccaagattacaaaattgaatcttaatactctgaaattgacattggaactttaataactcaaatattttaataaatgattagttgatgacttcttcactgattgatagtttaattatcttcccacagggatgtttcttgcggagctaatagaaaagtattttgtgtctcctaccctgttccgagtcatccgcctggccaggattggacgaatcctacgcctgatcaaaggtgccaaggggatccgcacgctgctctttgctctgatgatgtcccttcctgcgctgtttaacatcggcctcctgctttttctcgtcatgttcatctacgccatctttgggatgtccaactttgcctatgttaagagggaagttgggattgatgacatgttcaactttgagaccttcggcaacagcatgatctgcctgttccaaatcaccacctctgcgggctgggatggactgctggcccccatcctcaacagcaaaccccctgactgtgaccctaataaagttaaccctggaagctcggtgaagggagactgtgggaacccatctgtggggattttcttttttgtcagctacatcatcatatccttcctggttgtggtgaacatgtacattgctgtcatcctggagaacttcagcgttgccacagaagaaagtgcagagcctctgagtgaggacgactttgagatgttctacgaggtctgggagaagttcgaccctgacgccacccagttcatggaatttgaaaaattatctcagtttgcagctgctctagaaccccctctcaatttgccacaaccaaacaaacttcagctcattgccatggacctgcccatggtgagtggagaccgcatccactgcctggacatcttatttgcttttacaaagcgggtgttgggtgagagtggagagatggatgctcttcgaatccagatggaagagcggttcatggcttccaacccctccaaggtctcttatcagcccatcactactacattaaaacgcaaacaagaggaggtgtcagctgttatcattcagcgagcttataggcgccaccttttgaagcgaacagtaaaacaagcttcattcacatacaataagaacaaactcaaaggtggggctaatcttcttgtaaaagaagacatgctcattgacagaataaacgaaaactctattacggagaaaactgacctgacaatgtccacagcagcttgtccgccctcctacgatcgggtgacaaagccaatcgtggagaaacacgagcaggaagggaaggatgaaaaagccaaagggaaatgaacaaaaacaacaaaaaaaaataataaattgggtgacaaattgtttacagcctgtgaagtcaaaaggactcctttaagaagtcaatgccaaactgactgtttttacacaaattcactttaaggtcagtgcctcaataagacagtgacccccttgtcggcaaactctgtgactgtgtaaaggggagataaccttgacaggagggtactgttctaactaccagctgacactgctgaagacaagagacaaatggctatgcagactgtagggatcagtctaaaggggtgcagagctataatttgggggttaatgtgaaacactttagtgtagtaactctatccaccgtttgcatttcaactgccacatttgtcaggttttttacaaactctgttagtggattcatcttttttttaatccatgtgttgtttgttacatgtgactatttttgtaagcagggtttctgttgggaaatagactaaaggaactcttttcccggtgtgtcacctgggagtgttgacatccttatggccctcccgtctgcacaacggtgtggtttcgcatgagggcatgttgcaccttaggatcatgcatgagaaaaagtcgaaaggaaagcaaagacagttcttcaacagttctatgtcatctgtgtttctgcggggggagggggcggtacggaggtgatcgctagatcgtgggctttgctgagctgggtttgtcaaaatctagtccttagtccaaggagattgttcagacaggtcatcaagtcttaggaggtgcaaacttcattcaaatgtctggagtcacgctacatttctgttcactgtctctgaaaggtctctaaattatgactctgactccctacccccaaccctagcagagcgaattgaccaaaagaaccctttataaatccccgcttcctcctgcactttgtttagccatcttcagctctcagcaaggttgacactgtatgtgttaatgaaatgctatttattatgtaaatagtcattttactctgtggtgcacgtttgagcaaacaaataatgacgtaagcacagtatttattgcatcaaatatgtaccacgagaaatgtagagtgcaagctgtccaccggaaatatactgtactctgtaccattgtagatagtgtggatgctgccaatgcatgtcgatattaccacgttgcggtatctagtttctctcagcactcagaatctcactcacgagaaaccataggtcagtggctaagtcaaggaaactgttcagcagacttcatttctttaagtcattaagcaatagtttgcagcactttaacaatgtttgggttgtttttaaaatttaagtggataacgttcaatgtatagccagactgtacatacgtgttcaaaagacaacactactgcttaacctgttaaaaacatgtttagagttttataagcaaatataaatactgtacaaagtcactttcttttacttttcagcattatgtacatatgaagaggaaattatcttcaggttgatatcacaatcacttttcttactttctgtccacagcagtttttcatggaagatatttgctaactaagaaatgaaactatgactgggtagtggtagatctctgcttttttattacaattgttaattttggattatttcataattttaaggggcaaaatacactcacaacttcacatccaaattatgatttgcaattggaaaaggtataacatttgatttatatttattgtagtgtttgtgctaacagatgaccagagtgcttttttttctcatttttttctttatttccccaatttctgtttatttttctttgctttgaaatcatgcttctttagattgttctaaatataaatgtgggcttcagaatttcttttcctcaaaatcagagaggattacacttatatagtcaattacatcaagatatttgtgtttcttacagaagcagaccgtaggcacctctttttcttaaaaaaaaaatatttaggcaaaccgtattcatgaactgcatgttggaaaatgctactattagcccaaaatgatgctaatcacaattaaaaaatgtgcaaatctaataaaattaattttttattttattgcttgcctggttactttttgttgaccgaattttatgacaatatagaggtgcttttatctacttatggtagggagagttgcacacagacacacacacacacacacacacaccaatctttaagcactattactgggcagatattcatctattctaacccttaaactatctacttcccagccaaaggccccatgttccttgcagtttggatctcactggcttgctctgttccatgtgtgtcctcgtggagaatcccttggcaacctgctcattatgactcctcctggaccctctgactcatggccacctcccccttcttttcctcatggtccttcttttacttctgtctacttgggatcccctggctgggatcattagtccagccttctatctcctctgcccagccataggctgaatctgctctttattgagcaatcaaagatgagggaaaacaaatttttgtataacattgagatcagagttgccttggtgagatggctcagcggttaagagcattgactgctattcctaaggtcctgagttcaaatcccagcaaccacatggtggctcacaaccatctgtcatgaggccctcttctggtgtctaaagacagctacagtgtacttatgtataatatttttttttccattttttattaggtatttaactcatttacatttccaatgctataccaaaagtcccccatatccacccacccccactcccctgcccacccactccccctttttggccctggtattcccctgtactggggcatataaagtttgcaagtccaatgggcctctctttccagtgatggccgactaggccatcttttgatatatatgcagctagagtcaagagctccggggtactggttagctcataatgttgttccacctatagggttgcagatccctttagctccttggctactttctctagctcctccattgggagcccacttatgtataatattaaataaatcttttttagaaagaagaagaagaaagagatcagagttgcttaaccatgccaacttttggactgtagacagaagtctggacatagaaatcaacatctgaatatatataggctacaagaccaacccccagtacaccacacacacacacacacacacacacacacacacacacacacacatacacacacacacacacacacacacacaatagaacagaaaacttaaaagtaaaaattggacttcctagaatggcccctcagcagctagcagtagcaagatggaatctctaagagaaccaggagccaggactaacacataaactaatatccacgcaggggaggggagttatccaactcaacgcaacagggatggccacttgagatacataaagaagcctttaaagcatcactgatgaattcaacgttgctaagagtgggccaccatgttcctccagaagctccccacaccctctctgtgtttcttctgagaaagaggacctctgtacacatgtatatgttccgccgtcctttagttcccagttcgcttagccaatgggaagcctgggctggacatcaatacaaaactgtgagataggaatatctatccacctgttcctttcttgcaggtcttcttactccctgaaagtcagcatgccccgtcttacaactggagcaactttctgtgaagcgtcatagccatccttcttctcccctcgctttcttggatctgaggatgcttccttcctccttacgacttcccctatagttgctgacagcggtgaaaatgatttctttatgaagacctctctatgcctcattttaccatgccatgtgtcccttgccaggcagaactagctcctcaaagccagcaagaagcaatgattcaccttaggttggcttggagactttatctaactgactttcacagtcacagaacatgtcactgagagaaatacagcatggttacgcttggaattcacagacatggagagcttattagagtggttcaggtagcaaaactgaccatctaattatctgtgtgtgatggttagtatatgcttggcccaggggtgacactatcaggaggtgtggccttgttggagtaggtgtgtcactgtgggtgtgggctttaataccctagtcctagcagccttcagatgaagatatagtaccctcagttcctcctggacaatgcctgccttaatgctgccatgctcccaccttgatgataatagactgaacctctgaacctgttagccagccccaactaaatgttgtccttataagagttgacttggttatggtgtctgttcacagcagtaaaaccccaactaagacactgcatctctaagaaaaaagaaactttcatttaacttcaggattgtaccatctcagtcatgaaaccagaaagggcaacaatagttttgaccagaaaagagtaggccagtgatttttccacctttctacatctttctgaccatatttgtttacaatagacatgcaacgtaagagggcaactccaatttacaagttaaaagtttcattttcaaagaaagacaattagaacaatgagcttatacaaggtctcattggtacttttcttcatacaaaaagaagaaatagtgaaatataagcctacaaatcgtcctcatattttgtaatatatgctatgccaaaaagtaatttatgtacagagaattatggggtcttgtaactaggatggaagaagcatctagaatatccatgtcataggctaaatctgggacaagcagaccacccaaatagaacacaacacat

SEQ ID NO: 4 - DNA, 23 bp, synthetic construct
  source 1..23: mol_type = other DNA
ccctaagagccttatcacgattt

SEQ ID NO: 5 - DNA, 20 bp, synthetic construct
  source 1..20: mol_type = other DNA
ggcaaaccagaagcacattc

SEQ ID NO: 6 - DNA, 24 bp, synthetic construct
  source 1..24: mol_type = other DNA
agggtggttgtgaatgccctgtta

SEQ ID NO: 7 - DNA, 21 bp, synthetic construct
  source 1..21: mol_type = other DNA
agccctttattatgggtggtt

SEQ ID NO: 8 - DNA, 24 bp, synthetic construct
  source 1..24: mol_type = other DNA
ccagaatataaggcaaaccagaag

SEQ ID NO: 9 - DNA, 25 bp, synthetic construct
  source 1..25: mol_type = other DNA
tggatggaattgctcctaacagggc

SEQ ID NO: 10 - DNA, 20 bp, synthetic construct
  source 1..20: mol_type = other DNA
ggcaaattcaacggcacagt

SEQ ID NO: 11 - DNA, 20 bp, synthetic construct
  source 1..20: mol_type = other DNA
gggtctcgctcctggaagat

SEQ ID NO: 12 - DNA, 27 bp, synthetic construct
  source 1..27: mol_type = other DNA
aaggccgagaatgggaagcttgtcatc

SEQ ID NO: 13 - DNA, 18 bp, synthetic construct
  source 1..18: mol_type = other DNA
agttggagcaagattatc

SEQ ID NO: 14 - DNA, 19 bp, synthetic construct
  source 1..19: mol_type = other DNA
aagttggagcaagattatc

SEQ ID NO: 15 - DNA, 20 bp, synthetic construct
  source 1..20: mol_type = other DNA
aagttggagcaagattatcc

SEQ ID NO: 16 - DNA, 64 bp, Homo sapiens
  source 1..64: mol_type = genomic DNA
gataatcttgctccaacttggatggggtggagcgctggttcctcccctgagccctttattatgg

SEQ ID NO: 17 - DNA, 64 bp, Mus musculus
  source 1..64: mol_type = genomic DNA
gataatcttgctccaacttggatggggtggagcggtggttcctcccctcagccctttattatgg

SEQ ID NO: 18 - DNA, 23 bp, synthetic construct
  source 1..23: mol_type = other DNA
ggtagcaaaaggggtaatacagt

SEQ ID NO: 19 - DNA, 25 bp, synthetic construct
  source 1..25: mol_type = other DNA
atccaagttggagcaagattatcct

SEQ ID NO: 20 - DNA, 25 bp, synthetic construct
  source 1..25: mol_type = other DNA
tccaagttggagcaagattatccta

SEQ ID NO: 21 - DNA, 25 bp, synthetic construct
  source 1..25: mol_type = other DNA
ccaagttggagcaagattatcctat

SEQ ID NO: 22 - DNA, 25 bp, synthetic construct
  source 1..25: mol_type = other DNA
caagttggagcaagattatcctata

SEQ ID NO: 23 - DNA, 18 bp, synthetic construct
  source 1..18: mol_type = other DNA
  modified_base 1: mod_base = OTHER; note = 2'-O-(N-methylacetamide) adenosine 3'-phosphorothioate
  modified_base 2: mod_base = OTHER; note = 2'-O-(N-methylacetamide) guanosine 3'-phosphodiester
  modified_base 3: mod_base = OTHER; note = 2'-O-(N-methylacetamide) thymidine 3'-phosphodiester
  modified_base 4: mod_base = OTHER; note = 2'-O-(N-methylacetamide) thymidine 3'-phosphorothioate
  modified_base 5..6: mod_base = OTHER; note = 2'-O-(N-methylacetamide) guanosine 3'-phosphorothioate
  modified_base 7: mod_base = OTHER; note = 2'-O-(N-methylacetamide) adenosine 3'-phosphorothioate
  modified_base 8: mod_base = OTHER; note = 2'-O-(N-methylacetamide) guanosine 3'-phosphorothioate
  modified_base 9: mod_base = OTHER; note = 2'-O-(N-methylacetamide) 5-methylcytidine 3'-phosphorothioate
  modified_base 10..11: mod_base = OTHER; note = 2'-O-(N-methylacetamide) adenosine 3'-phosphorothioate
  modified_base 12: mod_base = OTHER; note = 2'-O-(N-methylacetamide) guanosine 3'-phosphorothioate
  modified_base 13: mod_base = OTHER; note = 2'-O-(N-methylacetamide) adenosine 3'-phosphorothioate
  modified_base 14..15: mod_base = OTHER; note = 2'-O-(N-methylacetamide) thymidine 3'-phosphorothioate
  modified_base 16: mod_base = OTHER; note = 2'-O-(N-methylacetamide) adenosine 3'-phosphorothioate
  modified_base 17: mod_base = OTHER; note = 2'-O-(N-methylacetamide) thymidine 3'-phosphorothioate
  modified_base 18: mod_base = OTHER; note = 2'-O-(N-methylacetamide) 5-methylcytidine
agttggagcaagattatc

SEQ ID NO: 24 - DNA, 18 bp, synthetic construct
  source 1..18: mol_type = other DNA
  modified_base 1: mod_base = OTHER; note = 2'-O-(N-methylacetamide) adenosine 3'-phosphorothioate
  modified_base 2: mod_base = OTHER; note = 2'-O-(N-methylacetamide) guanosine 3'-phosphodiester
  modified_base 3: mod_base = OTHER; note = 2'-O-(N-methylacetamide) thymidine 3'-phosphorothioate
  modified_base 4: mod_base = OTHER; note = 2'-O-(N-methylacetamide) thymidine 3'-phosphodiester
  modified_base 5..6: mod_base = OTHER; note = 2'-O-(N-methylacetamide) guanosine 3'-phosphorothioate
  modified_base 7: mod_base = OTHER; note = 2'-O-(N-methylacetamide) adenosine 3'-phosphorothioate
  modified_base 8: mod_base = OTHER; note = 2'-O-(N-methylacetamide) guanosine 3'-phosphorothioate
  modified_base 9: mod_base = OTHER; note = 2'-O-(N-methylacetamide) 5-methylcytidine 3'-phosphorothioate
  modified_base 10..11: mod_base = OTHER; note = 2'-O-(N-methylacetamide) adenosine 3'-phosphorothioate
  modified_base 12: mod_base = OTHER; note = 2'-O-(N-methylacetamide) guanosine 3'-phosphorothioate
  modified_base 13: mod_base = OTHER; note = 2'-O-(N-methylacetamide) adenosine 3'-phosphorothioate
  modified_base 14..15: mod_base = OTHER; note = 2'-O-(N-methylacetamide) thymidine 3'-phosphorothioate
  modified_base 16: mod_base = OTHER; note = 2'-O-(N-methylacetamide) adenosine 3'-phosphorothioate
  modified_base 17: mod_base = OTHER; note = 2'-O-(N-methylacetamide) thymidine 3'-phosphorothioate
  modified_base 18: mod_base = OTHER; note = 2'-O-(N-methylacetamide) 5-methylcytidine
agttggagcaagattatc

SEQ ID NO: 25 - DNA, 18 bp, synthetic construct
  source 1..18: mol_type = other DNA
  modified_base 1: mod_base = OTHER; note = 2'-O-(N-methylacetamide) adenosine 3'-phosphorothioate
  modified_base 2: mod_base = OTHER; note = 2'-O-(N-methylacetamide) guanosine 3'-phosphodiester
  modified_base 3..4: mod_base = OTHER; note = 2'-O-(N-methylacetamide) thymidine 3'-phosphorothioate
  modified_base 5: mod_base = OTHER; note = 2'-O-(N-methylacetamide) guanosine 3'-phosphodiester
  modified_base 6: mod_base = OTHER; note = 2'-O-(N-methylacetamide) guanosine 3'-phosphorothioate
  modified_base 7: mod_base = OTHER; note = 2'-O-(N-methylacetamide) adenosine 3'-phosphorothioate
  modified_base 8: mod_base = OTHER; note = 2'-O-(N-methylacetamide) guanosine 3'-phosphorothioate
  modified_base 9: mod_base = OTHER; note = 2'-O-(N-methylacetamide) 5-methylcytidine 3'-phosphorothioate
  modified_base 10..11: mod_base = OTHER; note = 2'-O-(N-methylacetamide) adenosine 3'-phosphorothioate
  modified_base 12: mod_base = OTHER; note = 2'-O-(N-methylacetamide) guanosine 3'-phosphorothioate
  modified_base 13: mod_base = OTHER; note = 2'-O-(N-methylacetamide) adenosine 3'-phosphorothioate
  modified_base 14..15: mod_base = OTHER; note = 2'-O-(N-methylacetamide) thymidine 3'-phosphorothioate
  modified_base 16: mod_base = OTHER; note = 2'-O-(N-methylacetamide) adenosine 3'-phosphorothioate
  modified_base 17: mod_base = OTHER; note = 2'-O-(N-methylacetamide) thymidine 3'-phosphorothioate
  modified_base 18: mod_base = OTHER; note = 2'-O-(N-methylacetamide) 5-methylcytidine
agttggagcaagattatc

SEQ ID NO: 26 - DNA, 18 bp, synthetic construct
  source 1..18: mol_type = other DNA
  modified_base 1: mod_base = OTHER; note = 2'-O-(N-methylacetamide) adenosine 3'-phosphorothioate
  modified_base 2: mod_base = OTHER; note = 2'-O-(N-methylacetamide) guanosine 3'-phosphodiester
  modified_base 3..4: mod_base = OTHER; note = 2'-O-(N-methylacetamide) thymidine 3'-phosphorothioate
  modified_base 5: mod_base = OTHER; note = 2'-O-(N-methylacetamide) guanosine 3'-phosphorothioate
  modified_base 6: mod_base = OTHER; note = 2'-O-(N-methylacetamide) guanosine 3'-phosphodiester
  modified_base 7: mod_base = OTHER; note = 2'-O-(N-methylacetamide) adenosine 3'-phosphorothioate
  modified_base 8: mod_base = OTHER; note = 2'-O-(N-methylacetamide) guanosine 3'-phosphorothioate
  modified_base 9: mod_base = OTHER; note = 2'-O-(N-methylacetamide) 5-methylcytidine 3'-phosphorothioate
  modified_base 10..11: mod_base = OTHER; note = 2'-O-(N-methylacetamide) adenosine 3'-phosphorothioate
  modified_base 12: mod_base = OTHER; note = 2'-O-(N-methylacetamide) guanosine 3'-phosphorothioate
  modified_base 13: mod_base = OTHER; note = 2'-O-(N-methylacetamide) adenosine 3'-phosphorothioate
  modified_base 14..15: mod_base = OTHER; note = 2'-O-(N-methylacetamide) thymidine 3'-phosphorothioate
  modified_base 16: mod_base = OTHER; note = 2'-O-(N-methylacetamide) adenosine 3'-phosphorothioate
  modified_base 17: mod_base = OTHER; note = 2'-O-(N-methylacetamide) thymidine 3'-phosphorothioate
  modified_base 18: mod_base = OTHER; note = 2'-O-(N-methylacetamide) 5-methylcytidine
agttggagcaagattatc

SEQ ID NO: 27 - DNA, 18 bp, synthetic construct
  source 1..18: mol_type = other DNA
  modified_base 1: mod_base = OTHER; note = 2'-O-(N-methylacetamide) adenosine 3'-phosphorothioate
  modified_base 2: mod_base = OTHER; note = 2'-O-(N-methylacetamide) guanosine 3'-phosphodiester
  modified_base 3..4: mod_base = OTHER; note = 2'-O-(N-methylacetamide) thymidine 3'-phosphorothioate
  modified_base 5..6: mod_base = OTHER; note = 2'-O-(N-methylacetamide) guanosine 3'-phosphorothioate
  modified_base 7: mod_base = OTHER; note = 2'-O-(N-methylacetamide) adenosine 3'-phosphorothioate
  modified_base 8: mod_base = OTHER; note = 2'-O-(N-methylacetamide) guanosine 3'-phosphodiester
  modified_base 9: mod_base = OTHER; note = 2'-O-(N-methylacetamide) 5-methylcytidine 3'-phosphorothioate
  modified_base 10..11: mod_base = OTHER; note = 2'-O-(N-methylacetamide) adenosine 3'-phosphorothioate
  modified_base 12: mod_base = OTHER; note = 2'-O-(N-methylacetamide) guanosine 3'-phosphorothioate
  modified_base 13: mod_base = OTHER; note = 2'-O-(N-methylacetamide) adenosine 3'-phosphorothioate
  modified_base 14..15: mod_base = OTHER; note = 2'-O-(N-methylacetamide) thymidine 3'-phosphorothioate
  modified_base 16: mod_base = OTHER; note = 2'-O-(N-methylacetamide) adenosine 3'-phosphorothioate
  modified_base 17: mod_base = OTHER; note = 2'-O-(N-methylacetamide) thymidine 3'-phosphorothioate
  modified_base 18: mod_base = OTHER; note = 2'-O-(N-methylacetamide) 5-methylcytidine
agttggagcaagattatc

SEQ ID NO: 28 - DNA, 18 bp, synthetic construct
  source 1..18: mol_type = other DNA
  modified_base 1: mod_base = OTHER; note = 2'-O-(N-methylacetamide) adenosine 3'-phosphorothioate
  modified_base 2: mod_base = OTHER; note = 2'-O-(N-methylacetamide) guanosine 3'-phosphorothioate
  modified_base 3: mod_base = OTHER; note = 2'-O-(N-methylacetamide) thymidine 3'-phosphorothioate
  modified_base 4: mod_base = OTHER; note = 2'-O-(N-methylacetamide) thymidine 3'-phosphodiester
  modified_base 5: mod_base = OTHER; note = 2'-O-(N-methylacetamide) guanosine 3'-phosphodiester
  modified_base 6: mod_base = OTHER; note = 2'-O-(N-methylacetamide) guanosine 3'-phosphorothioate
  modified_base 7: mod_base = OTHER; note = 2'-O-(N-methylacetamide) adenosine 3'-phosphorothioate
  modified_base 8: mod_base = OTHER; note = 2'-O-(N-methylacetamide) guanosine 3'-phosphorothioate
  modified_base 9: mod_base = OTHER; note = 2'-O-(N-methylacetamide) 5-methylcytidine 3'-phosphorothioate
  modified_base 10..11: mod_base = OTHER; note = 2'-O-(N-methylacetamide) adenosine 3'-phosphorothioate
  modified_base 12: mod_base = OTHER; note = 2'-O-(N-methylacetamide) guanosine 3'-phosphorothioate
  modified_base 13: mod_base = OTHER; note = 2'-O-(N-methylacetamide) adenosine 3'-phosphorothioate
  modified_base 14..15: mod_base = OTHER; note = 2'-O-(N-methylacetamide) thymidine 3'-phosphorothioate
  modified_base 16: mod_base = OTHER; note = 2'-O-(N-methylacetamide) adenosine 3'-phosphorothioate
  modified_base 17: mod_base = OTHER; note = 2'-O-(N-methylacetamide) thymidine 3'-phosphorothioate
  modified_base 18: mod_base = OTHER; note = 2'-O-(N-methylacetamide) 5-methylcytidine
agttggagcaagattatc

SEQ ID NO: 29 - DNA, 18 bp, synthetic construct
  source 1..18: mol_type = other DNA
  modified_base 1: mod_base = OTHER; note = 2'-O-(N-methylacetamide) adenosine 3'-phosphorothioate
  modified_base 2: mod_base = OTHER; note = 2'-O-(N-methylacetamide) guanosine 3'-phosphorothioate
  modified_base 3..4: mod_base = OTHER; note = 2'-O-(N-methylacetamide) thymidine 3'-phosphorothioate
  modified_base 5..6: mod_base = OTHER; note = 2'-O-(N-methylacetamide) guanosine 3'-phosphorothioate
  modified_base 7: mod_base = OTHER; note = 2'-O-(N-methylacetamide) adenosine 3'-phosphorothioate
  modified_base 8: mod_base = OTHER; note = 2'-O-(N-methylacetamide) guanosine 3'-phosphodiester
  modified_base 9: mod_base = OTHER; note = 2'-O-(N-methylacetamide) 5-methylcytidine 3'-phosphodiester
  modified_base 10..11: mod_base = OTHER; note = 2'-O-(N-methylacetamide) adenosine 3'-phosphorothioate
  modified_base 12: mod_base = OTHER; note = 2'-O-(N-methylacetamide) guanosine 3'-phosphorothioate
  modified_base 13: mod_base = OTHER; note = 2'-O-(N-methylacetamide) adenosine 3'-phosphorothioate
  modified_base 14..15: mod_base = OTHER; note = 2'-O-(N-methylacetamide) thymidine 3'-phosphorothioate
  modified_base 16: mod_base = OTHER; note = 2'-O-(N-methylacetamide) adenosine 3'-phosphorothioate
  modified_base 17: mod_base = OTHER; note = 2'-O-(N-methylacetamide) thymidine 3'-phosphorothioate
  modified_base 18: mod_base = OTHER; note = 2'-O-(N-methylacetamide) 5-methylcytidine
agttggagcaagattatc

SEQ ID NO: 30 - DNA, 18 bp, synthetic construct
  source 1..18: mol_type = other DNA
  modified_base 1: mod_base = OTHER; note = 2'-O-(N-methylacetamide) adenosine 3'-phosphorothioate
  modified_base 2: mod_base = OTHER; note = 2'-O-(N-methylacetamide) guanosine 3'-phosphorothioate
  modified_base 3..4: mod_base = OTHER; note = 2'-O-(N-methylacetamide) thymidine 3'-phosphorothioate
  modified_base 5..6: mod_base = OTHER; note = 2'-O-(N-methylacetamide) guanosine 3'-phosphorothioate
  modified_base 7: mod_base = OTHER; note = 2'-O-(N-methylacetamide) adenosine 3'-phosphorothioate
  modified_base 8: mod_base = OTHER; note = 2'-O-(N-methylacetamide) guanosine 3'-phosphorothioate
  modified_base 9: mod_base = OTHER; note = 2'-O-(N-methylacetamide) 5-methylcytidine 3'-phosphorothioate
  modified_base 10..11: mod_base = OTHER; note = 2'-O-(N-methylacetamide) adenosine 3'-phosphodiester
  modified_base 12: mod_base = OTHER; note = 2'-O-(N-methylacetamide) guanosine 3'-phosphorothioate
  modified_base 13: mod_base = OTHER; note = 2'-O-(N-methylacetamide) adenosine 3'-phosphorothioate
  modified_base 14..15: mod_base = OTHER; note = 2'-O-(N-methylacetamide) thymidine 3'-phosphorothioate
  modified_base 16: mod_base = OTHER; note = 2'-O-(N-methylacetamide) adenosine 3'-phosphorothioate
  modified_base 17: mod_base = OTHER; note = 2'-O-(N-methylacetamide) thymidine 3'-phosphorothioate
  modified_base 18: mod_base = OTHER; note = 2'-O-(N-methylacetamide) 5-methylcytidine
agttggagcaagattatc

SEQ ID NO: 31 - DNA, 18 bp, synthetic construct
  source 1..18: mol_type = other DNA
  modified_base 1: mod_base = OTHER; note = 2'-O-(N-methylacetamide) adenosine 3'-phosphorothioate
  modified_base 2: mod_base = OTHER; note = 2'-O-(N-methylacetamide) guanosine 3'-phosphorothioate
  modified_base 3..4: mod_base = OTHER; note = 2'-O-(N-methylacetamide) thymidine 3'-phosphorothioate
  modified_base 5..6: mod_base = OTHER; note = 2'-O-(N-methylacetamide) guanosine 3'-phosphorothioate
  modified_base 7: mod_base = OTHER; note = 2'-O-(N-methylacetamide) adenosine 3'-phosphorothioate
  modified_base 8: mod_base = OTHER; note = 2'-O-(N-methylacetamide) guanosine 3'-phosphorothioate
  modified_base 9: mod_base = OTHER; note = 2'-O-(N-methylacetamide) 5-methylcytidine 3'-phosphorothioate
  modified_base 10..11: mod_base = OTHER; note = 2'-O-(N-methylacetamide) adenosine 3'-phosphorothioate
  modified_base 12: mod_base = OTHER; note = 2'-O-(N-methylacetamide) guanosine 3'-phosphodiester
  modified_base 13: mod_base = OTHER; note = 2'-O-(N-methylacetamide) adenosine 3'-phosphodiester
  modified_base 14..15: mod_base = OTHER; note = 2'-O-(N-methylacetamide) thymidine 3'-phosphorothioate
  modified_base 16: mod_base = OTHER; note = 2'-O-(N-methylacetamide) adenosine 3'-phosphorothioate
  modified_base 17: mod_base = OTHER; note = 2'-O-(N-methylacetamide) thymidine 3'-phosphorothioate
  modified_base 18: mod_base = OTHER; note = 2'-O-(N-methylacetamide) 5-methylcytidine
agttggagcaagattatc

SEQ ID NO: 32 - DNA, 18 bp, synthetic construct
  source 1..18: mol_type = other DNA
  modified_base 1: mod_base = OTHER; note = 2'-O-(N-methylacetamide) adenosine 3'-phosphorothioate
  modified_base 2: mod_base = OTHER; note = 2'-O-(N-methylacetamide) guanosine 3'-phosphodiester
  modified_base 3..4: mod_base = OTHER; note = 2'-O-(N-methylacetamide) thymidine 3'-phosphorothioate
  modified_base 5..6: mod_base = OTHER; note = 2'-O-(N-methylacetamide) guanosine 3'-phosphorothioate
  modified_base 7: mod_base = OTHER; note = 2'-O-(N-methylacetamide) adenosine 3'-phosphorothioate
  modified_base 8: mod_base = OTHER; note = 2'-O-(N-methylacetamide) guanosine 3'-phosphorothioate
  modified_base 9: mod_base = OTHER; note = 2'-O-(N-methylacetamide) 5-methylcytidine 3'-phosphorothioate
  modified_base 10: mod_base = OTHER; note = 2'-O-(N-methylacetamide) adenosine 3'-phosphodiester
  modified_base 11: mod_base = OTHER; note = 2'-O-(N-methylacetamide) adenosine 3'-phosphorothioate
  modified_base 12: mod_base = OTHER; note = 2'-O-(N-methylacetamide) guanosine 3'-phosphorothioate
  modified_base 13: mod_base = OTHER; note = 2'-O-(N-methylacetamide) adenosine 3'-phosphorothioate
  modified_base 14..15: mod_base = OTHER; note = 2'-O-(N-methylacetamide) thymidine 3'-phosphorothioate
  modified_base 16: mod_base = OTHER; note = 2'-O-(N-methylacetamide) adenosine 3'-phosphorothioate
  modified_base 17: mod_base = OTHER; note = 2'-O-(N-methylacetamide) thymidine 3'-phosphorothioate
  modified_base 18: mod_base = OTHER; note = 2'-O-(N-methylacetamide) 5-methylcytidine
agttggagcaagattatc

SEQ ID NO: 33 - DNA, 18 bp, synthetic construct
  source 1..18: mol_type = other DNA
  modified_base 1: mod_base = OTHER; note = 2'-O-(N-methylacetamide) adenosine 3'-phosphorothioate
  modified_base 2: mod_base = OTHER; note = 2'-O-(N-methylacetamide) guanosine 3'-phosphodiester
  modified_base 3..4: mod_base = OTHER; note = 2'-O-(N-methylacetamide) thymidine 3'-phosphorothioate
  modified_base 5..6: mod_base = OTHER; note = 2'-O-(N-methylacetamide) guanosine 3'-phosphorothioate
  modified_base 7: mod_base = OTHER; note = 2'-O-(N-methylacetamide) adenosine 3'-phosphorothioate
  modified_base 8: mod_base = OTHER; note = 2'-O-(N-methylacetamide) guanosine 3'-phosphorothioate
  modified_base 9: mod_base = OTHER; note = 2'-O-(N-methylacetamide) 5-methylcytidine 3'-phosphorothioate
  modified_base 10..11: mod_base = OTHER; note = 2'-O-(N-methylacetamide) adenosine 3'-phosphorothioate
  modified_base 12: mod_base = OTHER; note = 2'-O-(N-methylacetamide) guanosine 3'-phosphodiester
  modified_base 13: mod_base = OTHER; note = 2'-O-(N-methylacetamide) adenosine 3'-phosphorothioate
  modified_base 14..15: mod_base = OTHER; note = 2'-O-(N-methylacetamide) thymidine 3'-phosphorothioate
  modified_base 16: mod_base = OTHER; note = 2'-O-(N-methylacetamide) adenosine 3'-phosphorothioate
  modified_base 17: mod_base = OTHER; note = 2'-O-(N-methylacetamide) thymidine 3'-phosphorothioate
  modified_base 18: mod_base = OTHER; note = 2'-O-(N-methylacetamide) 5-methylcytidine
agttggagcaagattatc

SEQ ID NO: 34 - DNA, 18 bp, synthetic construct
  source 1..18: mol_type = other DNA
  modified_base 1: mod_base = OTHER; note = 2'-O-(N-methylacetamide) adenosine 3'-phosphorothioate
  modified_base 2: mod_base = OTHER; note = 2'-O-(N-methylacetamide) guanosine 3'-phosphodiester
  modified_base 3..4: mod_base = OTHER; note = 2'-O-(N-methylacetamide) thymidine 3'-phosphorothioate
  modified_base 5..6: mod_base = OTHER; note = 2'-O-(N-methylacetamide) guanosine 3'-phosphorothioate
  modified_base 7: mod_base = OTHER; note = 2'-O-(N-methylacetamide) adenosine 3'-phosphorothioate
  modified_base 8: mod_base = OTHER; note = 2'-O-(N-methylacetamide) guanosine 3'-phosphorothioate
  modified_base 9: mod_base = OTHER; note = 2'-O-(N-methylacetamide) 5-methylcytidine 3'-phosphorothioate
  modified_base 10..11: mod_base = OTHER; note = 2'-O-(N-methylacetamide) adenosine 3'-phosphorothioate
  modified_base 12: mod_base = OTHER; note = 2'-O-(N-methylacetamide) guanosine 3'-phosphorothioate
  modified_base 13: mod_base = OTHER; note = 2'-O-(N-methylacetamide) adenosine 3'-phosphorothioate
  modified_base 14: mod_base = OTHER; note = 2'-O-(N-methylacetamide) thymidine 3'-phosphorothioate
  modified_base 15: mod_base = OTHER; note = 2'-O-(N-methylacetamide) thymidine 3'-phosphodiester
  modified_base 16: mod_base = OTHER; note = 2'-O-(N-methylacetamide) adenosine 3'-phosphorothioate
  modified_base 17: mod_base = OTHER; note = 2'-O-(N-methylacetamide) thymidine 3'-phosphorothioate
  modified_base 18: mod_base = OTHER; note = 2'-O-(N-methylacetamide) 5-methylcytidine
agttggagcaagattatc

SEQ ID NO: 35 - DNA, 18 bp, synthetic construct
  source 1..18: mol_type = other DNA
  modified_base 1: mod_base = OTHER; note = 2'-O-(N-methylacetamide) adenosine 3'-phosphorothioate
  modified_base 2: mod_base = OTHER; note = 2'-O-(N-methylacetamide) guanosine 3'-phosphorothioate
  modified_base 3..4: mod_base = OTHER; note = 2'-O-(N-methylacetamide) thymidine 3'-phosphorothioate
  modified_base 5: mod_base = OTHER; note = 2'-O-(N-methylacetamide) guanosine 3'-phosphorothioate
  modified_base 6: mod_base = OTHER; note = 2'-O-(N-methylacetamide) guanosine 3'-phosphodiester
  modified_base 7: mod_base = OTHER; note = 2'-O-(N-methylacetamide) adenosine 3'-phosphodiester
  modified_base 8: mod_base = OTHER; note = 2'-O-(N-methylacetamide) guanosine 3'-phosphorothioate
  modified_base 9: mod_base = OTHER; note = 2'-O-(N-methylacetamide) 5-methylcytidine 3'-phosphorothioate
  modified_base 10..11: mod_base = OTHER; note = 2'-O-(N-methylacetamide) adenosine 3'-phosphorothioate
  modified_base 12: mod_base = OTHER; note = 2'-O-(N-methylacetamide) guanosine 3'-phosphorothioate
  modified_base 13: mod_base = OTHER; note = 2'-O-(N-methylacetamide) adenosine 3'-phosphorothioate
  modified_base 14..15: mod_base = OTHER; note = 2'-O-(N-methylacetamide) thymidine 3'-phosphorothioate
  modified_base 16: mod_base = OTHER; note = 2'-O-(N-methylacetamide) adenosine 3'-phosphorothioate
  modified_base 17: mod_base = OTHER; note = 2'-O-(N-methylacetamide) thymidine 3'-phosphorothioate
  modified_base 18: mod_base = OTHER; note = 2'-O-(N-methylacetamide) 5-methylcytidine
agttggagcaagattatc

SEQ ID NO: 36 - DNA, 18 bp, synthetic construct
  source 1..18: mol_type = other DNA
  modified_base 1: mod_base = OTHER; note = 2'-O-(N-methylacetamide) adenosine 3'-phosphorothioate
  modified_base 2: mod_base = OTHER; note = 2'-O-(N-methylacetamide) guanosine 3'-phosphorothioate
  modified_base 3..4: mod_base = OTHER; note = 2'-O-(N-methylacetamide) thymidine 3'-phosphorothioate
  modified_base 5..6: mod_base = OTHER; note = 2'-O-(N-methylacetamide) guanosine 3'-phosphorothioate
  modified_base 7: mod_base = OTHER; note = 2'-O-(N-methylacetamide) adenosine 3'-phosphorothioate
  modified_base 8: mod_base = OTHER; note = 2'-O-(N-methylacetamide) guanosine 3'-phosphorothioate
  modified_base 9: mod_base = OTHER; note = 2'-O-(N-methylacetamide) 5-methylcytidine 3'-phosphorothioate
  modified_base 10..11: mod_base = OTHER; note = 2'-O-(N-methylacetamide) adenosine 3'-phosphorothioate
  modified_base 12: mod_base = OTHER; note = 2'-O-(N-methylacetamide) guanosine 3'-phosphorothioate
  modified_base 13: mod_base = OTHER; note = 2'-O-(N-methylacetamide) adenosine 3'-phosphorothioate
  modified_base 14..15: mod_base = OTHER; note = 2'-O-(N-methylacetamide) thymidine 3'-phosphodiester
  modified_base 16: mod_base = OTHER; note = 2'-O-(N-methylacetamide) adenosine 3'-phosphorothioate
  modified_base 17: mod_base = OTHER; note = 2'-O-(N-methylacetamide) thymidine 3'-phosphorothioate
  modified_base 18: mod_base = OTHER; note = 2'-O-(N-methylacetamide) 5-methylcytidine
agttggagcaagattatc

SEQ ID NO: 37 - DNA, 18 bp, synthetic construct
  source 1..18: mol_type = other DNA
  modified_base 1: mod_base = OTHER; note = 2'-O-(N-methylacetamide) adenosine 3'-phosphorothioate
  modified_base 2: mod_base = OTHER; note = 2'-O-(N-methylacetamide) guanosine 3'-phosphorothioate
  modified_base 3..4: mod_base = OTHER; note = 2'-O-(N-methylacetamide) thymidine 3'-phosphorothioate
  modified_base 5..6: mod_base = OTHER; note = 2'-O-(N-methylacetamide) guanosine 3'-phosphorothioate
  modified_base 7: mod_base = OTHER; note = 2'-O-(N-methylacetamide) adenosine 3'-phosphodiester
  modified_base 8: mod_base = OTHER; note = 2'-O-(N-methylacetamide) guanosine 3'-phosphorothioate
  modified_base 9: mod_base = OTHER; note = 2'-O-(N-methylacetamide) 5-methylcytidine 3'-phosphorothioate
  modified_base 10..11: mod_base = OTHER; note = 2'-O-(N-methylacetamide) adenosine 3'-phosphorothioate
  modified_base 12: mod_base = OTHER; note = 2'-O-(N-methylacetamide) guanosine 3'-phosphorothioate
  modified_base 13: mod_base = OTHER; note = 2'-O-(N-methylacetamide) adenosine 3'-phosphorothioate
  modified_base 14: mod_base = OTHER; note = 2'-O-(N-methylacetamide) thymidine 3'-phosphorothioate
  modified_base 15: mod_base = OTHER; note = 2'-O-(N-methylacetamide) thymidine 3'-phosphodiester
  modified_base 16: mod_base = OTHER; note = 2'-O-(N-methylacetamide) adenosine 3'-phosphorothioate
  modified_base 17: mod_base = OTHER; note = 2'-O-(N-methylacetamide) thymidine 3'-phosphorothioate
  modified_base 18: mod_base = OTHER; note = 2'-O-(N-methylacetamide) 5-methylcytidine
agttggagcaagattatc

SEQ ID NO: 38 - DNA, 18 bp, synthetic construct
  source 1..18: mol_type = other DNA
  modified_base 1: mod_base = OTHER; note = 2'-O-(N-methylacetamide) adenosine 3'-phosphorothioate
  modified_base 2: mod_base = OTHER; note = 2'-O-(N-methylacetamide) guanosine 3'-phosphorothioate
  modified_base 3..4: mod_base = OTHER; note = 2'-O-(N-methylacetamide) thymidine 3'-phosphorothioate
  modified_base 5..6: mod_base = OTHER; note = 2'-O-(N-methylacetamide) guanosine 3'-phosphorothioate
  modified_base 7: mod_base = OTHER; note = 2'-O-(N-methylacetamide) adenosine 3'-phosphorothioate
  modified_base 8: mod_base = OTHER; note = 2'-O-(N-methylacetamide) guanosine 3'-phosphorothioate
  modified_base 9: mod_base = OTHER; note = 2'-O-(N-methylacetamide) 5-methylcytidine 3'-phosphodiester
  modified_base 10..11: mod_base = OTHER; note = 2'-O-(N-methylacetamide) adenosine 3'-phosphorothioate
  modified_base 12: mod_base = OTHER; note = 2'-O-(N-methylacetamide) guanosine 3'-phosphorothioate
  modified_base 13: mod_base = OTHER; note = 2'-O-(N-methylacetamide) adenosine 3'-phosphorothioate
  modified_base 14: mod_base = OTHER; note = 2'-O-(N-methylacetamide) thymidine 3'-phosphorothioate
  modified_base 15: mod_base = OTHER; note = 2'-O-(N-methylacetamide) thymidine 3'-phosphodiester
  modified_base 16: mod_base = OTHER; note = 2'-O-(N-methylacetamide) adenosine 3'-phosphorothioate
  modified_base 17: mod_base = OTHER; note = 2'-O-(N-methylacetamide) thymidine 3'-phosphorothioate
  modified_base 18: mod_base = OTHER; note = 2'-O-(N-methylacetamide) 5-methylcytidine
agttggagcaagattatc

SEQ ID NO: 39 - DNA, 18 bp, synthetic construct
  source 1..18: mol_type = other DNA
  modified_base 1: mod_base = OTHER; note = 2'-O-(N-methylacetamide) adenosine 3'-phosphorothioate
  modified_base 2: mod_base = OTHER; note = 2'-O-(N-methylacetamide) guanosine 3'-phosphorothioate
  modified_base 3..4: mod_base = OTHER; note = 2'-O-(N-methylacetamide) thymidine 3'-phosphorothioate
  modified_base 5..6: mod_base = OTHER; note = 2'-O-(N-methylacetamide) guanosine 3'-phosphorothioate
  modified_base 7: mod_base = OTHER; note = 2'-O-(N-methylacetamide) adenosine 3'-phosphorothioate
  modified_base 8: mod_base = OTHER; note = 2'-O-(N-methylacetamide) guanosine 3'-phosphorothioate
  modified_base 9: mod_base = OTHER; note = 2'-O-(N-methylacetamide) 5-methylcytidine 3'-phosphorothioate
  modified_base 10: mod_base = OTHER; note = 2'-O-(N-methylacetamide) adenosine 3'-phosphorothioate
  modified_base 11: mod_base = OTHER; note = 2'-O-(N-methylacetamide) adenosine 3'-phosphodiester
  modified_base 12: mod_base = OTHER; note = 2'-O-(N-methylacetamide) guanosine 3'-phosphorothioate
  modified_base 13: mod_base = OTHER; note = 2'-O-(N-methylacetamide) adenosine 3'-phosphorothioate
  modified_base 14: mod_base = OTHER; note = 2'-O-(N-methylacetamide) thymidine 3'-phosphorothioate
  modified_base 15: mod_base = OTHER; note = 2'-O-(N-methylacetamide) thymidine 3'-phosphodiester
  modified_base 16: mod_base = OTHER; note = 2'-O-(N-methylacetamide) adenosine 3'-phosphorothioate
  modified_base 17: mod_base = OTHER; note = 2'-O-(N-methylacetamide) thymidine 3'-phosphorothioate
  modified_base 18: mod_base = OTHER; note = 2'-O-(N-methylacetamide) 5-methylcytidine
agttggagcaagattatc

SEQ ID NO: 40 - DNA, 18 bp, synthetic construct
  source 1..18: mol_type = other DNA
  modified_base 1: mod_base = OTHER; note = 2'-O-(N-methylacetamide) adenosine 3'-phosphorothioate
  modified_base 2: mod_base = OTHER; note = 2'-O-(N-methylacetamide) guanosine 3'-phosphorothioate
  modified_base 3..4: mod_base = OTHER; note = 2'-O-(N-methylacetamide) thymidine 3'-phosphorothioate
  modified_base 5..6: mod_base = OTHER; note = 2'-O-(N-methylacetamide) guanosine 3'-phosphorothioate
  modified_base 7: mod_base = OTHER; note = 2'-O-(N-methylacetamide) adenosine 3'-phosphorothioate
  modified_base 8: mod_base = OTHER; note = 2'-O-(N-methylacetamide) guanosine 3'-phosphorothioate
  modified_base 9: mod_base = OTHER; note = 2'-O-(N-methylacetamide) 5-methylcytidine 3'-phosphorothioate
  modified_base 10..11: mod_base = OTHER; note = 2'-O-(N-methylacetamide) adenosine 3'-phosphorothioate
  modified_base 12: mod_base = OTHER; note = 2'-O-(N-methylacetamide) guanosine 3'-phosphorothioate
  modified_base 13: mod_base = OTHER; note = 2'-O-(N-methylacetamide) adenosine 3'-phosphodiester
  modified_base 14: mod_base = OTHER; note = 2'-O-(N-methylacetamide) thymidine 3'-phosphorothioate
  modified_base 15: mod_base = OTHER; note = 2'-O-(N-methylacetamide) thymidine 3'-phosphodiester
  modified_base 16: mod_base = OTHER; note = 2'-O-(N-methylacetamide) adenosine 3'-phosphorothioate
  modified_base 17: mod_base = OTHER; note = 2'-O-(N-methylacetamide) thymidine 3'-phosphorothioate
  modified_base 18: mod_base = OTHER; note = 2'-O-(N-methylacetamide) 5-methylcytidine
agttggagcaagattatc

SEQ ID NO: 41 - DNA, 18 bp, synthetic construct
  source 1..18: mol_type = other DNA
  modified_base 1: mod_base = OTHER; note = 2'-O-(N-methylacetamide) adenosine 3'-phosphorothioate
  modified_base 2: mod_base = OTHER; note = 2'-O-(N-methylacetamide) guanosine 3'-phosphorothioate
  modified_base 3..4: mod_base = OTHER; note = 2'-O-(N-methylacetamide) thymidine 3'-phosphorothioate
  modified_base 5..6: mod_base = OTHER; note = 2'-O-(N-methylacetamide) guanosine 3'-phosphorothioate
  modified_base 7: mod_base = OTHER; note = 2'-O-(N-methylacetamide) adenosine 3'-phosphorothioate
  modified_base 8: mod_base = OTHER; note = 2'-O-(N-methylacetamide) guanosine 3'-phosphorothioate
  modified_base 9: mod_base = OTHER; note = 2'-O-(N-methylacetamide) 5-methylcytidine 3'-phosphodiester
  modified_base 10..11: mod_base = OTHER; note = 2'-O-(N-methylacetamide) adenosine 3'-phosphorothioate
  modified_base 12: mod_base = OTHER; note = 2'-O-(N-methylacetamide) guanosine 3'-phosphorothioate
  modified_base 13: mod_base = OTHER; note = 2'-O-(N-methylacetamide) adenosine 3'-phosphorothioate
  modified_base 14: mod_base = OTHER; note = 2'-O-(N-methylacetamide) thymidine 3'-phosphodiester
  modified_base 15: mod_base = OTHER; note = 2'-O-(N-methylacetamide) thymidine 3'-phosphorothioate
  modified_base 16: mod_base = OTHER; note = 2'-O-(N-methylacetamide) adenosine 3'-phosphorothioate
  modified_base 17: mod_base = OTHER; note = 2'-O-(N-methylacetamide) thymidine 3'-phosphorothioate
  modified_base 18: mod_base = OTHER; note = 2'-O-(N-methylacetamide) 5-methylcytidine
agttggagcaagattatc

SEQ ID NO: 42 - DNA, 18 bp, synthetic construct
  source 1..18: mol_type = other DNA
  modified_base 1: mod_base = OTHER; note = 2'-O-(N-methylacetamide) adenosine 3'-phosphorothioate
  modified_base 2: mod_base = OTHER; note = 2'-O-(N-methylacetamide) guanosine 3'-phosphorothioate
  modified_base 3..4: mod_base = OTHER; note = 2'-O-(N-methylacetamide) thymidine 3'-phosphorothioate
  modified_base 5..6: mod_base = OTHER; note = 2'-O-(N-methylacetamide) guanosine 3'-phosphorothioate
  modified_base 7: mod_base = OTHER; note = 2'-O-(N-methylacetamide) adenosine 3'-phosphorothioate
  modified_base 8: mod_base = OTHER; note = 2'-O-(N-methylacetamide) guanosine 3'-phosphorothioate
  modified_base 9: mod_base = OTHER; note = 2'-O-(N-methylacetamide) 5-methylcytidine 3'-phosphorothioate
  modified_base 10..11: mod_base = OTHER; note = 2'-O-(N-methylacetamide) adenosine 3'-phosphorothioate
  modified_base 12: mod_base = OTHER; note = 2'-O-(N-methylacetamide) guanosine 3'-phosphodiester
  modified_base 13: mod_base = OTHER; note = 2'-O-(N-methylacetamide) adenosine 3'-phosphorothioate
  modified_base 14: mod_base = OTHER; note = 2'-O-(N-methylacetamide) thymidine 3'-phosphodiester
  modified_base 15: mod_base = OTHER; note = 2'-O-(N-methylacetamide) thymidine 3'-phosphorothioate
  modified_base 16: mod_base = OTHER; note = 2'-O-(N-methylacetamide) adenosine 3'-phosphorothioate
  modified_base 17: mod_base = OTHER; note = 2'-O-(N-methylacetamide) thymidine 3'-phosphorothioate
  modified_base 18: mod_base = OTHER; note = 2'-O-(N-methylacetamide) 5-methylcytidine
agttggagcaagattatc

SEQ ID NO: 43 - DNA, 19 bp, synthetic construct
  source 1..19: mol_type = other DNA
  modified_base 1: mod_base = OTHER; note = 2'-O-(N-methylacetamide) adenosine 3'-phosphodiester
  modified_base 2: mod_base = OTHER; note = 2'-O-(N-methylacetamide) adenosine 3'-phosphorothioate
  modified_base 3: mod_base = OTHER; note = 2'-O-(N-methylacetamide) guanosine 3'-phosphorothioate
  modified_base 4..5: mod_base = OTHER; note = 2'-O-(N-methylacetamide) thymidine 3'-phosphorothioate
  modified_base 6..7: mod_base = OTHER; note = 2'-O-(N-methylacetamide) guanosine 3'-phosphorothioate
  modified_base 8: mod_base = OTHER; note = 2'-O-(N-methylacetamide) adenosine 3'-phosphorothioate
  modified_base 9: mod_base = OTHER; note = 2'-O-(N-methylacetamide) guanosine 3'-phosphorothioate
  modified_base 10: mod_base = OTHER; note = 2'-O-(N-methylacetamide) 5-methylcytidine 3'-phosphorothioate
  modified_base 11..12: mod_base = OTHER; note = 2'-O-(N-methylacetamide) adenosine 3'-phosphorothioate
  modified_base 13: mod_base = OTHER; note = 2'-O-(N-methylacetamide) guanosine 3'-phosphorothioate
  modified_base 14: mod_base = OTHER; note = 2'-O-(N-methylacetamide) adenosine 3'-phosphorothioate
  modified_base 15..16: mod_base = OTHER; note = 2'-O-(N-methylacetamide) thymidine 3'-phosphorothioate
  modified_base 17: mod_base = OTHER; note = 2'-O-(N-methylacetamide) adenosine 3'-phosphorothioate
  modified_base 18: mod_base = OTHER; note = 2'-O-(N-methylacetamide) thymidine 3'-phosphorothioate
  modified_base 19: mod_base = OTHER; note = 2'-O-(N-methylacetamide) 5-methylcytidine
aagttggagcaagattatc

SEQ ID NO: 44 - DNA, 20 bp, synthetic construct
  source 1..20: mol_type = other DNA
  modified_base 1: mod_base = OTHER; note = 2'-O-(N-methylacetamide) adenosine 3'-phosphodiester
  modified_base 2: mod_base = OTHER; note = 2'-O-(N-methylacetamide) adenosine 3'-phosphorothioate
  modified_base 3: mod_base = OTHER; note = 2'-O-(N-methylacetamide) guanosine 3'-phosphorothioate
  modified_base 4..5: mod_base = OTHER; note = 2'-O-(N-methylacetamide) thymidine 3'-phosphorothioate
  modified_base 6..7: mod_base = OTHER; note = 2'-O-(N-methylacetamide) guanosine 3'-phosphorothioate
  modified_base 8: mod_base = OTHER; note = 2'-O-(N-methylacetamide) adenosine 3'-phosphorothioate
  modified_base 9: mod_base = OTHER; note = 2'-O-(N-methylacetamide) guanosine 3'-phosphorothioate
  modified_base 10: mod_base = OTHER; note = 2'-O-(N-methylacetamide) 5-methylcytidine 3'-phosphorothioate
  modified_base 11..12: mod_base = OTHER; note = 2'-O-(N-methylacetamide) adenosine 3'-phosphorothioate
  modified_base 13: mod_base = OTHER; note = 2'-O-(N-methylacetamide) guanosine 3'-phosphorothioate
  modified_base 14: mod_base = OTHER; note = 2'-O-(N-methylacetamide) adenosine 3'-phosphorothioate
  modified_base 15..16: mod_base = OTHER; note = 2'-O-(N-methylacetamide) thymidine 3'-phosphorothioate
  modified_base 17: mod_base = OTHER; note = 2'-O-(N-methylacetamide) adenosine 3'-phosphorothioate
  modified_base 18: mod_base = OTHER; note = 2'-O-(N-methylacetamide) thymidine 3'-phosphorothioate
  modified_base 19: mod_base = OTHER; note = 2'-O-(N-methylacetamide) 5-methylcytidine 3'-phosphodiester
  modified_base 20: mod_base = OTHER; note = 2'-O-(N-methylacetamide) 5-methylcytidine
aagttggagcaagattatcc

SEQ ID NO: 45 - DNA, 23 bp, synthetic construct
  source 1..23: mol_type = other DNA
  modified_base 1..2: mod_base = OTHER; note = 2'-O-(N-methylacetamide) guanosine 3'-phosphorothioate
  modified_base 3: mod_base = OTHER; note = 2'-O-(N-methylacetamide) thymidine 3'-phosphodiester
  modified_base 4: mod_base = OTHER; note = 2'-O-(N-methylacetamide) adenosine 3'-phosphodiester
  modified_base 5: mod_base = OTHER; note = 2'-O-(N-methylacetamide) guanosine 3'-phosphorothioate
  modified_base 6: mod_base = OTHER; note = 2'-O-(N-methylacetamide) 5-methylcytidine 3'-phosphorothioate
  modified_base 7..10: mod_base = OTHER; note = 2'-O-(N-methylacetamide) adenosine 3'-phosphorothioate
  modified_base 11..14: mod_base = OTHER; note = 2'-O-(N-methylacetamide) guanosine 3'-phosphorothioate
  modified_base 15: mod_base = OTHER; note = 2'-O-(N-methylacetamide) thymidine 3'-phosphorothioate
  modified_base 16..17: mod_base = OTHER; note = 2'-O-(N-methylacetamide) adenosine 3'-phosphorothioate
  modified_base 18: mod_base = OTHER; note = 2'-O-(N-methylacetamide) thymidine 3'-phosphorothioate
  modified_base 19: mod_base = OTHER; note = 2'-O-(N-methylacetamide) adenosine 3'-phosphorothioate
  modified_base 20: mod_base = OTHER; note = 2'-O-(N-methylacetamide) 5-methylcytidine 3'-phosphorothioate
  modified_base 21: mod_base = OTHER; note = 2'-O-(N-methylacetamide) adenosine 3'-phosphorothioate
  modified_base 22: mod_base = OTHER; note = 2'-O-(N-methylacetamide) guanosine 3'-phosphorothioate
  modified_base 23: mod_base = OTHER; note = 2'-O-(N-methylacetamide) thymidine
ggtagcaaaaggggtaatacagt

SEQ ID NO: 46 - DNA, 23 bp, synthetic construct
  source 1..23: mol_type = other DNA
  modified_base 1..2: mod_base = OTHER; note = 2'-O-(N-methylacetamide) guanosine 3'-phosphorothioate
  modified_base 3: mod_base = OTHER; note = 2'-O-(N-methylacetamide) thymidine 3'-phosphorothioate
  modified_base 4: mod_base = OTHER; note = 2'-O-(N-methylacetamide) adenosine 3'-phosphorothioate
  modified_base 5: mod_base = OTHER; note = 2'-O-(N-methylacetamide) guanosine 3'-phosphorothioate
  modified_base 6: mod_base = OTHER; note = 2'-O-(N-methylacetamide) 5-methylcytidine 3'-phosphorothioate
  modified_base 7..10: mod_base = OTHER; note = 2'-O-(N-methylacetamide) adenosine 3'-phosphorothioate
  modified_base 11..14: mod_base = OTHER; note = 2'-O-(N-methylacetamide) guanosine 3'-phosphorothioate
  modified_base 15: mod_base = OTHER; note = 2'-O-(N-methylacetamide) thymidine 3'-phosphorothioate
  modified_base 16..17: mod_base = OTHER; note = 2'-O-(N-methylacetamide) adenosine 3'-phosphorothioate
  modified_base 18: mod_base = OTHER; note = 2'-O-(N-methylacetamide) thymidine 3'-phosphorothioate
  modified_base 19: mod_base = OTHER; note = 2'-O-(N-methylacetamide) adenosine 3'-phosphorothioate
  modified_base 20: mod_base = OTHER; note = 2'-O-(N-methylacetamide) 5-methylcytidine 3'-phosphorothioate
  modified_base 21: mod_base = OTHER; note = 2'-O-(N-methylacetamide) adenosine 3'-phosphorothioate
  modified_base 22: mod_base = OTHER; note = 2'-O-(N-methylacetamide) guanosine 3'-phosphorothioate
  modified_base 23: mod_base = OTHER; note = 2'-O-(N-methylacetamide) thymidine
ggtagcaaaaggggtaatacagt

SEQ ID NO: 47 - DNA, 25 bp, synthetic construct
  source 1..25: mol_type = other DNA
  modified_base 1: mod_base = OTHER; note = 2'-O-(N-methylacetamide) adenosine 3'-phosphorothioate
  modified_base 2: mod_base = OTHER; note = 2'-O-(N-methylacetamide) thymidine 3'-phosphorothioate
  modified_base 3..4: mod_base = OTHER; note = 2'-O-(N-methylacetamide) 5-methylcytidine 3'-phosphodiester
  modified_base 5: mod_base = OTHER; note = 2'-O-(N-methylacetamide) adenosine 3'-phosphorothioate
  modified_base 6: mod_base = OTHER; note = 2'-O-(N-methylacetamide) adenosine 3'-phosphodiester
  modified_base 7: mod_base = OTHER; note = 2'-O-(N-methylacetamide) guanosine 3'-phosphodiester
  modified_base 8: mod_base = OTHER; note = 2'-O-(N-methylacetamide) thymidine 3'-phosphorothioate
  modified_base 9: mod_base = OTHER; note = 2'-O-(N-methylacetamide) thymidine 3'-phosphodiester
  modified_base 10: mod_base = OTHER; note = 2'-O-(N-methylacetamide) guanosine 3'-phosphodiester
  modified_base 11: mod_base = OTHER; note = 2'-O-(N-methylacetamide) guanosine 3'-phosphorothioate
  modified_base 12: mod_base = OTHER; note = 2'-O-(N-methylacetamide) adenosine 3'-phosphorothioate
  modified_base 13: mod_base = OTHER; note = 2'-O-(N-methylacetamide) guanosine 3'-phosphorothioate
  modified_base 14: mod_base = OTHER; note = 2'-O-(N-methylacetamide) 5-methylcytidine 3'-phosphorothioate
  modified_base 15..16: mod_base = OTHER; note = 2'-O-(N-methylacetamide) adenosine 3'-phosphorothioate
  modified_base 17: mod_base = OTHER; note = 2'-O-(N-methylacetamide) guanosine 3'-phosphorothioate
  modified_base 18: mod_base = OTHER; note = 2'-O-(N-methylacetamide) adenosine 3'-phosphorothioate
  modified_base 19..20: mod_base = OTHER; note = 2'-O-(N-methylacetamide) thymidine 3'-phosphorothioate
  modified_base 21: mod_base = OTHER; note = 2'-O-(N-methylacetamide) adenosine 3'-phosphorothioate
  modified_base 22: mod_base = OTHER; note = 2'-O-(N-methylacetamide) thymidine 3'-phosphorothioate
  modified_base 23..24: mod_base = OTHER; note = 2'-O-(N-methylacetamide) 5-methylcytidine 3'-phosphorothioate
  modified_base 25: mod_base = OTHER; note = 2'-O-(N-methylacetamide) thymidine
atccaagttggagcaagattatcct

SEQ ID NO: 48 - DNA, 25 bp, synthetic construct
  source 1..25: mol_type = other DNA
  modified_base 1: mod_base = OTHER; note = 2'-O-(N-methylacetamide) thymidine 3'-phosphorothioate
  modified_base 2: mod_base = OTHER; note = 2'-O-(N-methylacetamide) 5-methylcytidine 3'-phosphorothioate
  modified_base 3: mod_base = OTHER; note = 2'-O-(N-methylacetamide) 5-methylcytidine 3'-phosphodiester
  modified_base 4: mod_base = OTHER; note = 2'-O-(N-methylacetamide) adenosine 3'-phosphodiester
  modified_base 5: mod_base = OTHER; note = 2'-O-(N-methylacetamide) adenosine 3'-phosphorothioate
  modified_base 6: mod_base = OTHER; note = 2'-O-(N-methylacetamide) guanosine 3'-phosphodiester
  modified_base 7: mod_base = OTHER; note = 2'-O-(N-methylacetamide) thymidine 3'-phosphodiester
  modified_base 8: mod_base = OTHER; note = 2'-O-(N-methylacetamide) thymidine 3'-phosphorothioate
  modified_base 9..10: mod_base = OTHER; note = 2'-O-(N-methylacetamide) guanosine 3'-phosphodiester
  modified_base 11: mod_base = OTHER; note = 2'-O-(N-methylacetamide) adenosine 3'-phosphorothioate
  modified_base 12: mod_base = OTHER; note = 2'-O-(N-methylacetamide) guanosine 3'-phosphorothioate
  modified_base 13: mod_base = OTHER; note = 2'-O-(N-methylacetamide) 5-methylcytidine 3'-phosphorothioate
  modified_base 14..15: mod_base = OTHER; note = 2'-O-(N-methylacetamide) adenosine 3'-phosphorothioate
  modified_base 16: mod_base = OTHER; note = 2'-O-(N-methylacetamide) guanosine 3'-phosphorothioate
  modified_base 17: mod_base = OTHER; note = 2'-O-(N-methylacetamide) adenosine 3'-phosphorothioate
  modified_base 18..19: mod_base = OTHER; note = 2'-O-(N-methylacetamide) thymidine 3'-phosphorothioate
  modified_base 20: mod_base = OTHER; note = 2'-O-(N-methylacetamide) adenosine 3'-phosphorothioate
  modified_base 21: mod_base = OTHER; note = 2'-O-(N-methylacetamide) thymidine 3'-phosphorothioate
  modified_base 22..23: mod_base = OTHER; note = 2'-O-(N-methylacetamide) 5-methylcytidine 3'-phosphorothioate
  modified_base 24: mod_base = OTHER; note = 2'-O-(N-methylacetamide) thymidine 3'-phosphorothioate
  modified_base 25: mod_base = OTHER; note = 2'-O-(N-methylacetamide) adenosine
tccaagttggagcaagattatccta

SEQ ID NO: 49 - DNA, 25 bp, synthetic construct
  source 1..25: mol_type = other DNA
  modified_base 1..2: mod_base = OTHER; note = 2'-O-(N-methylacetamide) 5-methylcytidine 3'-phosphorothioate
  modified_base 3..4: mod_base = OTHER; note = 2'-O-(N-methylacetamide) adenosine 3'-phosphodiester
  modified_base 5: mod_base = OTHER; note = 2'-O-(N-methylacetamide) guanosine 3'-phosphorothioate
  modified_base 6..7: mod_base = OTHER; note = 2'-O-(N-methylacetamide) thymidine 3'-phosphodiester
  modified_base 8: mod_base = OTHER; note = 2'-O-(N-methylacetamide) guanosine 3'-phosphorothioate
  modified_base 9: mod_base = OTHER; note = 2'-O-(N-methylacetamide) guanosine 3'-phosphodiester
  modified_base 10: mod_base = OTHER; note = 2'-O-(N-methylacetamide) adenosine 3'-phosphodiester
  modified_base 11: mod_base = OTHER; note = 2'-O-(N-methylacetamide) guanosine 3'-phosphorothioate
  modified_base 12: mod_base = OTHER; note = 2'-O-(N-methylacetamide) 5-methylcytidine 3'-phosphorothioate
  modified_base 13..14: mod_base = OTHER; note = 2'-O-(N-methylacetamide) adenosine 3'-phosphorothioate
  modified_base 15: mod_base = OTHER; note = 2'-O-(N-methylacetamide) guanosine 3'-phosphorothioate
  modified_base 16: mod_base = OTHER; note = 2'-O-(N-methylacetamide) adenosine 3'-phosphorothioate
  modified_base 17..18: mod_base = OTHER; note = 2'-O-(N-methylacetamide) thymidine 3'-phosphorothioate
  modified_base 19: mod_base = OTHER; note = 2'-O-(N-methylacetamide) adenosine 3'-phosphorothioate
  modified_base 20: mod_base = OTHER; note = 2'-O-(N-methylacetamide) thymidine 3'-phosphorothioate
  modified_base 21..22: mod_base = OTHER; note = 2'-O-(N-methylacetamide) 5-methylcytidine 3'-phosphorothioate
  modified_base 23: mod_base = OTHER; note = 2'-O-(N-methylacetamide) thymidine 3'-phosphorothioate
  modified_base 24: mod_base = OTHER; note = 2'-O-(N-methylacetamide) adenosine 3'-phosphorothioate
  modified_base 25: mod_base = OTHER; note = 2'-O-(N-methylacetamide) thymidine
ccaagttggagcaagattatcctat

SEQ ID NO: 50 - DNA, 25 bp, synthetic construct
  source 1..25: mol_type = other DNA
  modified_base 1: mod_base = OTHER; note = 2'-O-(N-methylacetamide) 5-methylcytidine 3'-phosphorothioate
  modified_base 2: mod_base = OTHER; note = 2'-O-(N-methylacetamide) adenosine 3'-phosphorothioate
  modified_base 3: mod_base = OTHER; note = 2'-O-(N-methylacetamide) adenosine 3'-phosphodiester
  modified_base 4: mod_base = OTHER; note = 2'-O-(N-methylacetamide) guanosine 3'-phosphodiester
  modified_base 5: mod_base = OTHER; note = 2'-O-(N-methylacetamide) thymidine 3'-phosphorothioate
  modified_base 6: mod_base = OTHER; note = 2'-O-(N-methylacetamide) thymidine 3'-phosphodiester
  modified_base 7: mod_base = OTHER; note = 2'-O-(N-methylacetamide) guanosine 3'-phosphodiester
  modified_base 8: mod_base = OTHER; note = 2'-O-(N-methylacetamide) guanosine 3'-phosphorothioate
  modified_base 9: mod_base = OTHER; note = 2'-O-(N-methylacetamide) adenosine 3'-phosphodiester
  modified_base 10: mod_base = OTHER; note = 2'-O-(N-methylacetamide) guanosine 3'-phosphodiester
  modified_base 11: mod_base = OTHER; note = 2'-O-(N-methylacetamide) 5-methylcytidine 3'-phosphorothioate
  modified_base 12..13: mod_base = OTHER; note = 2'-O-(N-methylacetamide) adenosine 3'-phosphorothioate
  modified_base 14: mod_base = OTHER; note = 2'-O-(N-methylacetamide) guanosine 3'-phosphorothioate
  modified_base 15: mod_base = OTHER; note = 2'-O-(N-methylacetamide) adenosine 3'-phosphorothioate
  modified_base 16..17: mod_base = OTHER; note = 2'-O-(N-methylacetamide) thymidine 3'-phosphorothioate
  modified_base 18: mod_base = OTHER; note = 2'-O-(N-methylacetamide) adenosine 3'-phosphorothioate
  modified_base 19: mod_base = OTHER; note = 2'-O-(N-methylacetamide) thymidine 3'-phosphorothioate
  modified_base 20..21: mod_base = OTHER; note = 2'-O-(N-methylacetamide) 5-methylcytidine 3'-phosphorothioate
  modified_base 22: mod_base = OTHER; note = 2'-O-(N-methylacetamide) thymidine 3'-phosphorothioate
  modified_base 23: mod_base = OTHER; note = 2'-O-(N-methylacetamide) adenosine 3'-phosphorothioate
  modified_base 24: mod_base = OTHER; note = 2'-O-(N-methylacetamide) thymidine 3'-phosphorothioate
  modified_base 25: mod_base = OTHER; note = 2'-O-(N-methylacetamide) adenosine
caagttggagcaagattatcctata

SEQ ID NO: 51 - DNA, 25 bp, synthetic construct
  source 1..25: mol_type = other DNA
  modified_base 1: mod_base = OTHER; note = 2'-O-(N-methylacetamide) adenosine 3'-phosphorothioate
  modified_base 2: mod_base = OTHER; note = 2'-O-(N-methylacetamide) thymidine 3'-phosphorothioate
  modified_base 3..4: mod_base = OTHER; note = 2'-O-(N-methylacetamide) 5-methylcytidine 3'-phosphodiester
  modified_base 5: mod_base = OTHER; note = 2'-O-(N-methylacetamide) adenosine 3'-phosphorothioate
  modified_base 6: mod_base = OTHER; note = 2'-O-(N-methylacetamide) adenosine 3'-phosphodiester
  modified_base 7: mod_base = OTHER; note = 2'-O-(N-methylacetamide) guanosine 3'-phosphodiester
  modified_base 8..9: mod_base = OTHER; note = 2'-O-(N-methylacetamide) thymidine 3'-phosphorothioate
  modified_base 10..11: mod_base = OTHER; note = 2'-O-(N-methylacetamide) guanosine 3'-phosphorothioate
  modified_base 12: mod_base = OTHER; note = 2'-O-(N-methylacetamide) adenosine 3'-phosphorothioate
  modified_base 13: mod_base = OTHER; note = 2'-O-(N-methylacetamide) guanosine 3'-phosphorothioate
  modified_base 14: mod_base = OTHER; note = 2'-O-(N-methylacetamide) 5-methylcytidine 3'-phosphorothioate
  modified_base 15..16: mod_base = OTHER; note = 2'-O-(N-methylacetamide) adenosine 3'-phosphorothioate
  modified_base 17: mod_base = OTHER; note = 2'-O-(N-methylacetamide) guanosine 3'-phosphorothioate
  modified_base 18: mod_base = OTHER; note = 2'-O-(N-methylacetamide) adenosine 3'-phosphorothioate
  modified_base 19..20: mod_base = OTHER; note = 2'-O-(N-methylacetamide) thymidine 3'-phosphorothioate
  modified_base 21: mod_base = OTHER; note = 2'-O-(N-methylacetamide) adenosine 3'-phosphorothioate
  modified_base 22: mod_base = OTHER; note = 2'-O-(N-methylacetamide) thymidine 3'-phosphorothioate
  modified_base 23..24: mod_base = OTHER; note = 2'-O-(N-methylacetamide) 5-methylcytidine 3'-phosphorothioate
  modified_base 25: mod_base = OTHER; note = 2'-O-(N-methylacetamide) thymidine
atccaagttggagcaagattatcct

SEQ ID NO: 52 - DNA, 25 bp, synthetic construct
  source 1..25: mol_type = other DNA
  modified_base 1: mod_base = OTHER; note = 2'-O-(N-methylacetamide) thymidine 3'-phosphorothioate
  modified_base 2: mod_base = OTHER; note = 2'-O-(N-methylacetamide) 5-methylcytidine 3'-phosphorothioate
  modified_base 3: mod_base = OTHER; note = 2'-O-(N-methylacetamide) 5-methylcytidine 3'-phosphodiester
  modified_base 4: mod_base = OTHER; note = 2'-O-(N-methylacetamide) adenosine 3'-phosphodiester
  modified_base 5: mod_base = OTHER; note = 2'-O-(N-methylacetamide) adenosine 3'-phosphorothioate
  modified_base 6: mod_base = OTHER; note = 2'-O-(N-methylacetamide) guanosine 3'-phosphodiester
  modified_base 7: mod_base = OTHER; note = 2'-O-(N-methylacetamide) thymidine 3'-phosphodiester
  modified_base 8: mod_base = OTHER; note = 2'-O-(N-methylacetamide) thymidine 3'-phosphorothioate
  modified_base 9..10: mod_base = OTHER; note = 2'-O-(N-methylacetamide) guanosine 3'-phosphorothioate
  modified_base 11: mod_base = OTHER; note = 2'-O-(N-methylacetamide) adenosine 3'-phosphorothioate
  modified_base 12: mod_base = OTHER; note = 2'-O-(N-methylacetamide) guanosine 3'-phosphorothioate
  modified_base 13: mod_base = OTHER; note = 2'-O-(N-methylacetamide) 5-methylcytidine 3'-phosphorothioate
  modified_base 14..15: mod_base = OTHER; note = 2'-O-(N-methylacetamide) adenosine 3'-phosphorothioate
  modified_base 16: mod_base = OTHER; note = 2'-O-(N-methylacetamide) guanosine 3'-phosphorothioate
  modified_base 17: mod_base = OTHER; note = 2'-O-(N-methylacetamide) adenosine 3'-phosphorothioate
  modified_base 18..19: mod_base = OTHER; note = 2'-O-(N-methylacetamide) thymidine 3'-phosphorothioate
  modified_base 20: mod_base = OTHER; note = 2'-O-(N-methylacetamide) adenosine 3'-phosphorothioate
  modified_base 21: mod_base = OTHER; note = 2'-O-(N-methylacetamide) thymidine 3'-phosphorothioate
  modified_base 22..23: mod_base = OTHER; note = 2'-O-(N-methylacetamide) 5-methylcytidine 3'-phosphorothioate
  modified_base 24: mod_base = OTHER; note = 2'-O-(N-methylacetamide) thymidine 3'-phosphorothioate
  modified_base 25: mod_base = OTHER; note = 2'-O-(N-methylacetamide) adenosine
tccaagttggagcaagattatccta

SEQ ID NO: 53 - DNA, 25 bp, synthetic construct
  source 1..25: mol_type = other DNA
  modified_base 1..2: mod_base = OTHER; note = 2'-O-(N-methylacetamide) 5-methylcytidine 3'-phosphorothioate
  modified_base 3..4: mod_base = OTHER; note = 2'-O-(N-methylacetamide) adenosine 3'-phosphodiester
  modified_base 5: mod_base = OTHER; note = 2'-O-(N-methylacetamide) guanosine 3'-phosphorothioate
  modified_base 6..7: mod_base = OTHER; note = 2'-O-(N-methylacetamide) thymidine 3'-phosphodiester
  modified_base 8..9: mod_base = OTHER; note = 2'-O-(N-methylacetamide) guanosine 3'-phosphorothioate
  modified_base 10: mod_base = OTHER; note = 2'-O-(N-methylacetamide) adenosine 3'-phosphorothioate
  modified_base 11: mod_base = OTHER; note = 2'-O-(N-methylacetamide) guanosine 3'-phosphorothioate
  modified_base 12: mod_base = OTHER; note = 2'-O-(N-methylacetamide) 5-methylcytidine 3'-phosphorothioate
  modified_base 13..14: mod_base = OTHER; note = 2'-O-(N-methylacetamide) adenosine 3'-phosphorothioate
  modified_base 15: mod_base = OTHER; note = 2'-O-(N-methylacetamide) guanosine 3'-phosphorothioate
  modified_base 16: mod_base = OTHER; note = 2'-O-(N-methylacetamide) adenosine 3'-phosphorothioate
  modified_base 17..18: mod_base = OTHER; note = 2'-O-(N-methylacetamide) thymidine 3'-phosphorothioate
  modified_base 19: mod_base = OTHER; note = 2'-O-(N-methylacetamide) adenosine 3'-phosphorothioate
  modified_base 20: mod_base = OTHER; note = 2'-O-(N-methylacetamide) thymidine 3'-phosphorothioate
  modified_base 21..22: mod_base = OTHER; note = 2'-O-(N-methylacetamide) 5-methylcytidine 3'-phosphorothioate
  modified_base 23: mod_base = OTHER; note = 2'-O-(N-methylacetamide) thymidine 3'-phosphorothioate
  modified_base 24: mod_base = OTHER; note = 2'-O-(N-methylacetamide) adenosine 3'-phosphorothioate
  modified_base 25: mod_base = OTHER; note = 2'-O-(N-methylacetamide) thymidine
ccaagttggagcaagattatcctat

SEQ ID NO: 54 - DNA, 25 bp, synthetic construct
  source 1..25: mol_type = other DNA
  modified_base 1: mod_base = OTHER; note = 2'-O-(N-methylacetamide) 5-methylcytidine 3'-phosphorothioate
  modified_base 2: mod_base = OTHER; note = 2'-O-(N-methylacetamide) adenosine 3'-phosphorothioate
  modified_base 3: mod_base = OTHER; note = 2'-O-(N-methylacetamide) adenosine 3'-phosphodiester
  modified_base 4: mod_base = OTHER; note = 2'-O-(N-methylacetamide) guanosine 3'-phosphodiester
  modified_base 5: mod_base = OTHER; note = 2'-O-(N-methylacetamide) thymidine 3'-phosphorothioate
  modified_base 6: mod_base = OTHER; note = 2'-O-(N-methylacetamide) thymidine 3'-phosphodiester
  modified_base 7: mod_base = OTHER; note = 2'-O-(N-methylacetamide) guanosine 3'-phosphodiester
  modified_base 8: mod_base = OTHER; note = 2'-O-(N-methylacetamide) guanosine 3'-phosphorothioate
  modified_base 9: mod_base = OTHER; note = 2'-O-(N-methylacetamide) adenosine 3'-phosphorothioate
  modified_base 10: mod_base = OTHER; note = 2'-O-(N-methylacetamide) guanosine 3'-phosphorothioate
  modified_base 11: mod_base = OTHER; note = 2'-O-(N-methylacetamide) 5-methylcytidine 3'-phosphorothioate
  modified_base 12..13: mod_base = OTHER; note = 2'-O-(N-methylacetamide) adenosine 3'-phosphorothioate
  modified_base 14: mod_base = OTHER; note = 2'-O-(N-methylacetamide) guanosine 3'-phosphorothioate
  modified_base 15: mod_base = OTHER; note = 2'-O-(N-methylacetamide) adenosine 3'-phosphorothioate
  modified_base 16..17: mod_base = OTHER; note = 2'-O-(N-methylacetamide) thymidine 3'-phosphorothioate
  modified_base 18: mod_base = OTHER; note = 2'-O-(N-methylacetamide) adenosine 3'-phosphorothioate
  modified_base 19: mod_base = OTHER; note = 2'-O-(N-methylacetamide) thymidine 3'-phosphorothioate
  modified_base 20..21: mod_base = OTHER; note = 2'-O-(N-methylacetamide) 5-methylcytidine 3'-phosphorothioate
  modified_base 22: mod_base = OTHER; note = 2'-O-(N-methylacetamide) thymidine 3'-phosphorothioate
  modified_base 23: mod_base = OTHER; note = 2'-O-(N-methylacetamide) adenosine 3'-phosphorothioate
  modified_base 24: mod_base = OTHER; note = 2'-O-(N-methylacetamide) thymidine 3'-phosphorothioate
  modified_base 25: mod_base = OTHER; note = 2'-O-(N-methylacetamide) adenosine
caagttggagcaagattatcctata

SEQ ID NO: 55 - DNA, 25 bp, synthetic construct
  source 1..25: mol_type = other DNA
  modified_base 1: mod_base = OTHER; note = 2'-O-(N-methylacetamide) adenosine 3'-phosphorothioate
  modified_base 2: mod_base = OTHER; note = 2'-O-(N-methylacetamide) thymidine 3'-phosphorothioate
  modified_base 3..4: mod_base = OTHER; note = 2'-O-(N-methylacetamide) 5-methylcytidine 3'-phosphodiester
  modified_base 5..6: mod_base = OTHER; note = 2'-O-(N-methylacetamide) adenosine 3'-phosphorothioate
  modified_base 7: mod_base = OTHER; note = 2'-O-(N-methylacetamide) guanosine 3'-phosphorothioate
  modified_base 8..9: mod_base = OTHER; note = 2'-O-(N-methylacetamide) thymidine 3'-phosphorothioate
  modified_base 10..11: mod_base = OTHER; note = 2'-O-(N-methylacetamide) guanosine 3'-phosphorothioate
  modified_base 12: mod_base = OTHER; note = 2'-O-(N-methylacetamide) adenosine 3'-phosphorothioate
  modified_base 13: mod_base = OTHER; note = 2'-O-(N-methylacetamide) guanosine 3'-phosphorothioate
  modified_base 14: mod_base = OTHER; note = 2'-O-(N-methylacetamide) 5-methylcytidine 3'-phosphorothioate
  modified_base 15..16: mod_base = OTHER; note = 2'-O-(N-methylacetamide) adenosine 3'-phosphorothioate
  modified_base 17: mod_base = OTHER; note = 2'-O-(N-methylacetamide) guanosine 3'-phosphorothioate
  modified_base 18: mod_base = OTHER; note = 2'-O-(N-methylacetamide) adenosine 3'-phosphorothioate
  modified_base 19..20: mod_base = OTHER; note = 2'-O-(N-methylacetamide) thymidine 3'-phosphorothioate
  modified_base 21: mod_base = OTHER; note = 2'-O-(N-methylacetamide) adenosine 3'-phosphorothioate
  modified_base 22: mod_base = OTHER; note = 2'-O-(N-methylacetamide) thymidine 3'-phosphorothioate
  modified_base 23..24: mod_base = OTHER; note = 2'-O-(N-methylacetamide) 5-methylcytidine 3'-phosphorothioate
  modified_base 25: mod_base = OTHER; note = 2'-O-(N-methylacetamide) thymidine
atccaagttggagcaagattatcct

SEQ ID NO: 56 - DNA, 25 bp, synthetic construct
  source 1..25: mol_type = other DNA
  modified_base 1: mod_base = OTHER; note = 2'-O-(N-methylacetamide) thymidine 3'-phosphorothioate
  modified_base 2: mod_base = OTHER; note = 2'-O-(N-methylacetamide) 5-methylcytidine 3'-phosphorothioate
  modified_base 3: mod_base = OTHER; note = 2'-O-(N-methylacetamide) 5-methylcytidine 3'-phosphodiester
  modified_base 4: mod_base = OTHER; note = 2'-O-(N-methylacetamide) adenosine 3'-phosphodiester
  modified_base 5: mod_base = OTHER; note = 2'-O-(N-methylacetamide) adenosine 3'-phosphorothioate
  modified_base 6: mod_base = OTHER; note = 2'-O-(N-methylacetamide) guanosine 3'-phosphorothioate
  modified_base 7..8: mod_base = OTHER; note = 2'-O-(N-methylacetamide) thymidine 3'-phosphorothioate
  modified_base 9..10: mod_base = OTHER; note = 2'-O-(N-methylacetamide) guanosine 3'-phosphorothioate
  modified_base 11: mod_base = OTHER; note = 2'-O-(N-methylacetamide) adenosine 3'-phosphorothioate
  modified_base 12: mod_base = OTHER; note = 2'-O-(N-methylacetamide) guanosine 3'-phosphorothioate
  modified_base 13: mod_base = OTHER; note = 2'-O-(N-methylacetamide) 5-methylcytidine 3'-phosphorothioate
  modified_base 14..15: mod_base = OTHER; note = 2'-O-(N-methylacetamide) adenosine 3'-phosphorothioate
  modified_base 16: mod_base = OTHER; note = 2'-O-(N-methylacetamide) guanosine 3'-phosphorothioate
  modified_base 17: mod_base = OTHER; note = 2'-O-(N-methylacetamide) adenosine 3'-phosphorothioate
  modified_base 18..19: mod_base = OTHER; note = 2'-O-(N-methylacetamide) thymidine 3'-phosphorothioate
  modified_base 20: mod_base = OTHER; note = 2'-O-(N-methylacetamide) adenosine 3'-phosphorothioate
  modified_base 21: mod_base = OTHER; note = 2'-O-(N-methylacetamide) thymidine 3'-phosphorothioate
  modified_base 22..23: mod_base = OTHER; note = 2'-O-(N-methylacetamide) 5-methylcytidine 3'-phosphorothioate
  modified_base 24: mod_base = OTHER; note = 2'-O-(N-methylacetamide) thymidine 3'-phosphorothioate
  modified_base 25: mod_base = OTHER; note = 2'-O-(N-methylacetamide) adenosine
tccaagttggagcaagattatccta

SEQ ID NO: 57 - DNA, 25 bp, synthetic construct
  source 1..25: mol_type = other DNA
  modified_base 1..2: mod_base = OTHER; note = 2'-O-(N-methylacetamide) 5-methylcytidine 3'-phosphorothioate
  modified_base 3..4: mod_base = OTHER; note = 2'-O-(N-methylacetamide) adenosine 3'-phosphodiester
  modified_base 5: mod_base = OTHER; note = 2'-O-(N-methylacetamide) guanosine 3'-phosphorothioate
  modified_base 6..7: mod_base = OTHER; note = 2'-O-(N-methylacetamide) thymidine 3'-phosphorothioate
  modified_base 8..9: mod_base = OTHER; note = 2'-O-(N-methylacetamide) guanosine 3'-phosphorothioate
  modified_base 10: mod_base = OTHER; note = 2'-O-(N-methylacetamide) adenosine 3'-phosphorothioate
  modified_base 11: mod_base = OTHER; note = 2'-O-(N-methylacetamide) guanosine 3'-phosphorothioate
  modified_base 12: mod_base = OTHER; note = 2'-O-(N-methylacetamide) 5-methylcytidine 3'-phosphorothioate
  modified_base 13..14: mod_base = OTHER; note = 2'-O-(N-methylacetamide) adenosine 3'-phosphorothioate
  modified_base 15: mod_base = OTHER; note = 2'-O-(N-methylacetamide) guanosine 3'-phosphorothioate
  modified_base 16: mod_base = OTHER; note = 2'-O-(N-methylacetamide) adenosine 3'-phosphorothioate
  modified_base 17..18: mod_base = OTHER; note = 2'-O-(N-methylacetamide) thymidine 3'-phosphorothioate
  modified_base 19: mod_base = OTHER; note = 2'-O-(N-methylacetamide) adenosine 3'-phosphorothioate
  modified_base 20: mod_base = OTHER; note = 2'-O-(N-methylacetamide) thymidine 3'-phosphorothioate
  modified_base 21..22: mod_base = OTHER; note = 2'-O-(N-methylacetamide) 5-methylcytidine 3'-phosphorothioate
  modified_base 23: mod_base = OTHER; note = 2'-O-(N-methylacetamide) thymidine 3'-phosphorothioate
  modified_base 24: mod_base = OTHER; note = 2'-O-(N-methylacetamide) adenosine 3'-phosphorothioate
  modified_base 25: mod_base = OTHER; note = 2'-O-(N-methylacetamide) thymidine
ccaagttggagcaagattatcctat

SEQ ID NO: 58 - DNA, 25 bp, synthetic construct
  source 1..25: mol_type = other DNA
  modified_base 1: mod_base = OTHER; note = 2'-O-(N-methylacetamide) 5-methylcytidine 3'-phosphorothioate
  modified_base 2: mod_base = OTHER; note = 2'-O-(N-methylacetamide) adenosine 3'-phosphorothioate
  modified_base 3: mod_base = OTHER; note = 2'-O-(N-methylacetamide) adenosine 3'-phosphodiester
  modified_base 4: mod_base = OTHER; note = 2'-O-(N-methylacetamide) guanosine 3'-phosphodiester
  modified_base 5..6: mod_base = OTHER; note = 2'-O-(N-methylacetamide) thymidine 3'-phosphorothioate
  modified_base 7..8: mod_base = OTHER; note = 2'-O-(N-methylacetamide) guanosine 3'-phosphorothioate
  modified_base 9: mod_base = OTHER; note = 2'-O-(N-methylacetamide) adenosine 3'-phosphorothioate
  modified_base 10: mod_base = OTHER; note = 2'-O-(N-methylacetamide) guanosine 3'-phosphorothioate
  modified_base 11: mod_base = OTHER; note = 2'-O-(N-methylacetamide) 5-methylcytidine 3'-phosphorothioate
  modified_base 12..13: mod_base = OTHER; note = 2'-O-(N-methylacetamide) adenosine 3'-phosphorothioate
  modified_base 14: mod_base = OTHER; note = 2'-O-(N-methylacetamide) guanosine 3'-phosphorothioate
  modified_base 15: mod_base = OTHER; note = 2'-O-(N-methylacetamide) adenosine 3'-phosphorothioate
  modified_base 16..17: mod_base = OTHER; note = 2'-O-(N-methylacetamide) thymidine 3'-phosphorothioate
  modified_base 18: mod_base = OTHER; note = 2'-O-(N-methylacetamide) adenosine 3'-phosphorothioate
  modified_base 19: mod_base = OTHER; note = 2'-O-(N-methylacetamide) thymidine 3'-phosphorothioate
  modified_base 20..21: mod_base = OTHER; note = 2'-O-(N-methylacetamide) 5-methylcytidine 3'-phosphorothioate
  modified_base 22: mod_base = OTHER; note = 2'-O-(N-methylacetamide) thymidine 3'-phosphorothioate
  modified_base 23: mod_base = OTHER; note = 2'-O-(N-methylacetamide) adenosine 3'-phosphorothioate
  modified_base 24: mod_base = OTHER; note = 2'-O-(N-methylacetamide) thymidine 3'-phosphorothioate
  modified_base 25: mod_base = OTHER; note = 2'-O-(N-methylacetamide) adenosine
caagttggagcaagattatcctata

SEQ ID NO: 59 - DNA, 25 bp, synthetic construct
  source 1..25: mol_type = other DNA
  modified_base 1: mod_base = OTHER; note = 2'-O-(N-methylacetamide) adenosine 3'-phosphorothioate
  modified_base 2: mod_base = OTHER; note = 2'-O-(N-methylacetamide) thymidine 3'-phosphorothioate
  modified_base 3..4: mod_base = OTHER; note = 2'-O-(N-methylacetamide) 5-methylcytidine 3'-phosphorothioate
  modified_base 5..6: mod_base = OTHER; note = 2'-O-(N-methylacetamide) adenosine 3'-phosphorothioate
  modified_base 7: mod_base = OTHER; note = 2'-O-(N-methylacetamide) guanosine 3'-phosphorothioate
  modified_base 8..9: mod_base = OTHER; note = 2'-O-(N-methylacetamide) thymidine 3'-phosphorothioate
  modified_base 10..11: mod_base = OTHER; note = 2'-O-(N-methylacetamide) guanosine 3'-phosphorothioate
  modified_base 12: mod_base = OTHER; note = 2'-O-(N-methylacetamide) adenosine 3'-phosphorothioate
  modified_base 13: mod_base = OTHER; note = 2'-O-(N-methylacetamide) guanosine 3'-phosphorothioate
  modified_base 14: mod_base = OTHER; note = 2'-O-(N-methylacetamide) 5-methylcytidine 3'-phosphorothioate
  modified_base 15..16: mod_base = OTHER; note = 2'-O-(N-methylacetamide) adenosine 3'-phosphorothioate
  modified_base 17: mod_base = OTHER; note = 2'-O-(N-methylacetamide) guanosine 3'-phosphorothioate
  modified_base 18: mod_base = OTHER; note = 2'-O-(N-methylacetamide) adenosine 3'-phosphorothioate
  modified_base 19..20: mod_base = OTHER; note = 2'-O-(N-methylacetamide) thymidine 3'-phosphorothioate
  modified_base 21: mod_base = OTHER; note = 2'-O-(N-methylacetamide) adenosine 3'-phosphorothioate
  modified_base 22: mod_base = OTHER; note = 2'-O-(N-methylacetamide) thymidine 3'-phosphorothioate
  modified_base 23..24: mod_base = OTHER; note = 2'-O-(N-methylacetamide) 5-methylcytidine 3'-phosphorothioate
  modified_base 25: mod_base = OTHER; note = 2'-O-(N-methylacetamide) thymidine
atccaagttggagcaagattatcct

SEQ ID NO: 60 - DNA, 25 bp, synthetic construct
  source 1..25: mol_type = other DNA
  modified_base 1: mod_base = OTHER; note = 2'-O-(N-methylacetamide) thymidine 3'-phosphorothioate
  modified_base 2..3: mod_base = OTHER; note = 2'-O-(N-methylacetamide) 5-methylcytidine 3'-phosphorothioate
  modified_base 4..5: mod_base = OTHER; note = 2'-O-(N-methylacetamide) adenosine 3'-phosphorothioate
  modified_base 6: mod_base = OTHER; note = 2'-O-(N-methylacetamide) guanosine 3'-phosphorothioate
  modified_base 7..8: mod_base = OTHER; note = 2'-O-(N-methylacetamide) thymidine 3'-phosphorothioate
  modified_base 9..10: mod_base = OTHER; note = 2'-O-(N-methylacetamide) guanosine 3'-phosphorothioate
  modified_base 11: mod_base = OTHER; note = 2'-O-(N-methylacetamide) adenosine 3'-phosphorothioate
  modified_base 12: mod_base = OTHER; note = 2'-O-(N-methylacetamide) guanosine 3'-phosphorothioate
  modified_base 13: mod_base = OTHER; note = 2'-O-(N-methylacetamide) 5-methylcytidine 3'-phosphorothioate
  modified_base 14..15: mod_base = OTHER; note = 2'-O-(N-methylacetamide) adenosine 3'-phosphorothioate
  modified_base 16: mod_base = OTHER; note = 2'-O-(N-methylacetamide) guanosine 3'-phosphorothioate
  modified_base 17: mod_base = OTHER; note = 2'-O-(N-methylacetamide) adenosine 3'-phosphorothioate
  modified_base 18..19: mod_base = OTHER; note = 2'-O-(N-methylacetamide) thymidine 3'-phosphorothioate
  modified_base 20: mod_base = OTHER; note = 2'-O-(N-methylacetamide) adenosine 3'-phosphorothioate
  modified_base 21: mod_base = OTHER; note = 2'-O-(N-methylacetamide) thymidine 3'-phosphorothioate
  modified_base 22..23: mod_base = OTHER; note = 2'-O-(N-methylacetamide) 5-methylcytidine 3'-phosphorothioate
  modified_base 24: mod_base = OTHER; note = 2'-O-(N-methylacetamide) thymidine 3'-phosphorothioate
  modified_base 25: mod_base = OTHER; note = 2'-O-(N-methylacetamide) adenosine
tccaagttggagcaagattatccta

SEQ ID NO: 61 - DNA, 25 bp, synthetic construct
  source 1..25: mol_type = other DNA
  modified_base 1..2: mod_base = OTHER; note = 2'-O-(N-methylacetamide) 5-methylcytidine 3'-phosphorothioate
  modified_base 3..4: mod_base = OTHER; note = 2'-O-(N-methylacetamide) adenosine 3'-phosphorothioate
  modified_base 5: mod_base = OTHER; note = 2'-O-(N-methylacetamide) guanosine 3'-phosphorothioate
  modified_base 6..7: mod_base = OTHER; note = 2'-O-(N-methylacetamide) thymidine 3'-phosphorothioate
  modified_base 8..9: mod_base = OTHER; note = 2'-O-(N-methylacetamide) guanosine 3'-phosphorothioate
  modified_base 10: mod_base = OTHER; note = 2'-O-(N-methylacetamide) adenosine 3'-phosphorothioate
  modified_base 11: mod_base = OTHER; note = 2'-O-(N-methylacetamide) guanosine 3'-phosphorothioate
  modified_base 12: mod_base = OTHER; note = 2'-O-(N-methylacetamide) 5-methylcytidine 3'-phosphorothioate
  modified_base 13..14: mod_base = OTHER; note = 2'-O-(N-methylacetamide) adenosine 3'-phosphorothioate
  modified_base 15: mod_base = OTHER; note = 2'-O-(N-methylacetamide) guanosine 3'-phosphorothioate
  modified_base 16: mod_base = OTHER; note = 2'-O-(N-methylacetamide) adenosine 3'-phosphorothioate
  modified_base 17..18: mod_base = OTHER; note = 2'-O-(N-methylacetamide) thymidine 3'-phosphorothioate
  modified_base 19: mod_base = OTHER; note = 2'-O-(N-methylacetamide) adenosine 3'-phosphorothioate
  modified_base 20: mod_base = OTHER; note = 2'-O-(N-methylacetamide) thymidine 3'-phosphorothioate
  modified_base 21..22: mod_base = OTHER; note = 2'-O-(N-methylacetamide) 5-methylcytidine 3'-phosphorothioate
  modified_base 23: mod_base = OTHER; note = 2'-O-(N-methylacetamide) thymidine 3'-phosphorothioate
  modified_base 24: mod_base = OTHER; note = 2'-O-(N-methylacetamide) adenosine 3'-phosphorothioate
  modified_base 25: mod_base = OTHER; note = 2'-O-(N-methylacetamide) thymidine
ccaagttggagcaagattatcctat

SEQ ID NO: 62 - DNA, 25 bp, synthetic construct
  source 1..25: mol_type = other DNA
  modified_base 1: mod_base = OTHER; note = 2'-O-(N-methylacetamide) 5-methylcytidine 3'-phosphorothioate
  modified_base 2..3: mod_base = OTHER; note = 2'-O-(N-methylacetamide) adenosine 3'-phosphorothioate
  modified_base 4: mod_base = OTHER; note = 2'-O-(N-methylacetamide) guanosine 3'-phosphorothioate
  modified_base 5..6: mod_base = OTHER; note = 2'-O-(N-methylacetamide) thymidine 3'-phosphorothioate
  modified_base 7..8: mod_base = OTHER; note = 2'-O-(N-methylacetamide) guanosine 3'-phosphorothioate
  modified_base 9: mod_base = OTHER; note = 2'-O-(N-methylacetamide) adenosine 3'-phosphorothioate
  modified_base 10: mod_base = OTHER; note = 2'-O-(N-methylacetamide) guanosine 3'-phosphorothioate
  modified_base 11: mod_base = OTHER; note = 2'-O-(N-methylacetamide) 5-methylcytidine 3'-phosphorothioate
  modified_base 12..13: mod_base = OTHER; note = 2'-O-(N-methylacetamide) adenosine 3'-phosphorothioate
  modified_base 14: mod_base = OTHER; note = 2'-O-(N-methylacetamide) guanosine 3'-phosphorothioate
  modified_base 15: mod_base = OTHER; note = 2'-O-(N-methylacetamide) adenosine 3'-phosphorothioate
  modified_base 16..17: mod_base = OTHER; note = 2'-O-(N-methylacetamide) thymidine 3'-phosphorothioate
  modified_base 18: mod_base = OTHER; note = 2'-O-(N-methylacetamide) adenosine 3'-phosphorothioate
  modified_base 19: mod_base = OTHER; note = 2'-O-(N-methylacetamide) thymidine 3'-phosphorothioate
  modified_base 20..21: mod_base = OTHER; note = 2'-O-(N-methylacetamide) 5-methylcytidine 3'-phosphorothioate
  modified_base 22: mod_base = OTHER; note = 2'-O-(N-methylacetamide) thymidine 3'-phosphorothioate
  modified_base 23: mod_base = OTHER; note = 2'-O-(N-methylacetamide) adenosine 3'-phosphorothioate
  modified_base 24: mod_base = OTHER; note = 2'-O-(N-methylacetamide) thymidine 3'-phosphorothioate
  modified_base 25: mod_base = OTHER; note = 2'-O-(N-methylacetamide) adenosine
caagttggagcaagattatcctata

SEQ ID NO: 63 - DNA, 18 bp, synthetic construct
  source 1..18: mol_type = other DNA
ggtagcaaaaggggtaat

SEQ ID NO: 64 - DNA, 18 bp, synthetic construct
  source 1..18: mol_type = other DNA
agcaaaaggggtaataca

SEQ ID NO: 65 - DNA, 18 bp, synthetic construct
  source 1..18: mol_type = other DNA
gcaaaaggggtaatacag

SEQ ID NO: 66 - DNA, 18 bp, synthetic construct
  source 1..18: mol_type = other DNA
caaaaggggtaatacagt

SEQ ID NO: 67 - DNA, 18 bp, synthetic construct
  source 1..18: mol_type = other DNA
aaaggggtaatacagtac

SEQ ID NO: 68 - DNA, 18 bp, synthetic construct
  source 1..18: mol_type = other DNA
aaggggtaatacagtacc

SEQ ID NO: 69 - DNA, 18 bp, synthetic construct
  source 1..18: mol_type = other DNA
gtaatacagtacccataa

SEQ ID NO: 70 - DNA, 18 bp, synthetic construct
  source 1..18: mol_type = other DNA
taatacagtacccataat

SEQ ID NO: 71 - DNA, 18 bp, synthetic construct
  source 1..18: mol_type = other DNA
aatacagtacccataata

SEQ ID NO: 72 - DNA, 18 bp, synthetic construct
  source 1..18: mol_type = other DNA
atacagtacccataataa

SEQ ID NO: 73 - DNA, 18 bp, synthetic construct
  source 1..18: mol_type = other DNA
tacagtacccataataaa

SEQ ID NO: 74 - DNA, 18 bp, synthetic construct
  source 1..18: mol_type = other DNA
acagtacccataataaag

SEQ ID NO: 75 - DNA, 18 bp, synthetic construct
  source 1..18: mol_type = other DNA
cccatccaagttggagca

SEQ ID NO: 76 - DNA, 18 bp, synthetic construct
  source 1..18: mol_type = other DNA
ccatccaagttggagcaa

SEQ ID NO: 77 - DNA, 18 bp, synthetic construct
  source 1..18: mol_type = other DNA
catccaagttggagcaag

SEQ ID NO: 78 - DNA, 18 bp, synthetic construct
  source 1..18: mol_type = other DNA
atccaagttggagcaaga

SEQ ID NO: 79 - DNA, 18 bp, synthetic construct
  source 1..18: mol_type = other DNA
tccaagttggagcaagat

SEQ ID NO: 80 - DNA, 18 bp, synthetic construct
  source 1..18: mol_type = other DNA
ccaagttggagcaagatt

SEQ ID NO: 81 - DNA, 18 bp, synthetic construct
  source 1..18: mol_type = other DNA
caagttggagcaagatta

SEQ ID NO: 82 - DNA, 18 bp, synthetic construct
  source 1..18: mol_type = other DNA
aagttggagcaagattat

SEQ ID NO: 83 - DNA, 18 bp, synthetic construct
  source 1..18: mol_type = other DNA
ttggagcaagattatcct

SEQ ID NO: 84 - DNA, 18 bp, synthetic construct
  source 1..18: mol_type = other DNA
tggagcaagattatccta

SEQ ID NO: 85 - DNA, 18 bp, synthetic construct
  source 1..18: mol_type = other DNA
ggagcaagattatcctat

SEQ ID NO: 86 - DNA, 18 bp, synthetic construct
  source 1..18: mol_type = other DNA
gagcaagattatcctata

SEQ ID NO: 87 - DNA, 18 bp, synthetic construct
  source 1..18: mol_type = other DNA
agcaagattatcctatat

SEQ ID NO: 88 - DNA, 18 bp, synthetic construct
  source 1..18: mol_type = other DNA
gcaagattatcctatata

SEQ ID NO: 89 - DNA, 18 bp, synthetic construct
  source 1..18: mol_type = other DNA
aagattatcctatataaa